(19) United States
(12) Patent Application Publication
(10) Pub. No.: US 2010/0160369 A1
(43) Pub. Date: Jun. 24, 2010

(54) S1P1 Agonists and Methods of Making And Using

(75) Inventors: Lynne Canne Bannen, Lucerne, CA (US); Diva Sze-Ming Chan, Emeryville, CA (US); Xiao-Hui Gu, Potomac, MD (US); Morrison B. Mac, San Francisco, CA (US); Stephanie Ng, Palo Alto, CA (US); Tie-Lin Wang, San Diego, CA (US); Yong Wang, Foster City, CA (US); Wei Xu, Danville, CA (US)

Correspondence Address: MCDONNELL BOEHNEN HULBERT @ BERGHOFF LLP, 300 SOUTH WACKER DRIVE, SUITE 3100, CHICAGO, IL, 60606, US

(73) Assignee: EXELIXIS, INC., South San Francisco, CA (US)

(21) Appl. No.: 12/630,663

(22) Filed: Dec. 3, 2009

Related U.S. Application Data
(60) Provisional application No. 61/200,878, filed on Dec. 4, 2008.

(51) Int. Cl. A61K 31/437; C07D 471/04; A61P 17/06; A61P 37/00

(52) U.S. Cl. 514/300; 546/121

(57) ABSTRACT

The invention is directed to Compounds of Formula I:

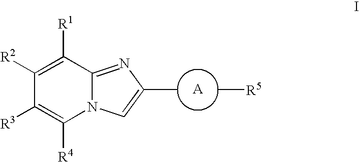

as well as methods of making and using the compounds.

### BACKGROUND OF THE INVENTION

1. Field of the Invention

This invention relates to the field of agonists of Sphingosine 1-Phosphate Type 1 Receptor (S1P1) (a lysophospholipid) and methods of using the agonists.

2. Summary of the Related Art

Sphingosine 1-phosphate (S1P) is a biologically active lysophospholipid that serves as a key regulator of cellular differentiation and survival.

Circulation of mature lymphocytes between blood and secondary lymphoid tissues plays an important role in the immune system. Agonism of S1P1R has been shown to lead to the sequestration of peripheral lymphocytes into secondary lymphoid tissue. Such sequestration of lymphocytes has been shown to result in immunosuppressive activity in animal models. Known S1P1 receptor agonists, such as FTY720, have been shown to markedly decrease peripheral blood lymphocytes through the sequestration of lymphocytes into secondary lymphoid tissues. Potent agonists of the S1P1 receptor are thought to induce long-term down-regulation of S1P1 on lymphocytes, thereby inhibiting the migration of lymphocytes toward S1P. The consequential decrease in trafficking and infiltration of antigen-specific T cells provides a means of immunomodulating activity that can be useful in the treatment of various immune-related conditions such as graft versus host disease and autoimmune diseases such as multiple sclerosis, rheumatoid arthritis, systemic lupus erythematosis, psoriasis, Grave's disease, myasthenia gravis, Crohn's disease, and ulcerative colitis. Therefore, agonists of S1P1R are potentially useful immunosuppressants for the treatment of a variety of autoimmune conditions.

### SUMMARY OF THE INVENTION

The following only summarizes certain aspects of the invention and is not intended to be limiting in nature. These aspects and other aspects and embodiments are described more fully below. All references cited in this specification are hereby incorporated by reference in their entirety. In the event of a discrepancy between the express disclosure of this specification and the references incorporated by reference, the express disclosure of this specification shall control.

The invention provides compounds that are S1P1 agonists and are useful in the treatment of graft versus host disease and autoimmune diseases (such as multiple sclerosis, rheumatoid arthritis, systemic lupus erythematosis, psoriasis, Grave's disease, myasthenia gravis, Crohn's disease, and ulcerative colitis) in mammals. This invention also provides methods of making the compound of the invention, methods of using such compounds in the treatment of graft versus host disease and autoimmune diseases, especially humans, and to pharmaceutical compositions containing such compounds. The invention also comprises methods of using the compounds for the in vivo study of the the role S1P1 in various biological processes, including graft versus host disease and autoimmune diseases.

A first aspect of the invention provides a compound of Formula I:

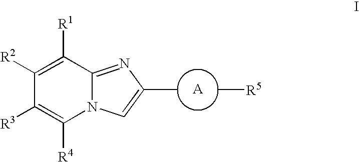

or a single stereoisomer or a mixture of isomers thereof, all optionally as a pharmaceutically acceptable salt thereof, where
R¹ is hydrogen, halo, cyano, alkoxy, amino, alkylamino, or dialkylamino;
R² is hydrogen, methyl, or methoxy;
R³ is hydrogen, alkyl, alkylsulfonyl, halo, haloalkyl, alkoxy, optionally substituted phenoxy, cyano, alkylsulfonylamino, or nitro;
R⁴ is hydrogen or alkyl;

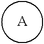

is a 5-membered heroarylene;
R⁵ is phenyl substituted with R⁶, R⁷, and R⁸; or
R⁵ is heteroaryl optionally substituted with one or two R¹⁵ groups independently selected from alkyl; carboxy; haloalkyl; carboxyalkyl; alkoxycarbonylalkyl; and alkyl substituted with one —C(O)NR¹⁴R^{14a} group where R¹⁴ is hydrogen, alkyl, haloalkyl, or hydroxyalkyl and R^{14a} is hydrogen, alkyl, haloalkyl, hydroxyalkyl, or alkyl substituted with —O—Si(alkyl)₃; provided that when the R⁵ heteroaryl is pyridinyl or thienyl, then the pyridinyl and thienyl is substituted with one R¹⁵ and optionally substituted with an independently selected second R¹⁵.
R⁶ is halo; hydroxy; cyano; —C(O)H; carboxy; alkoxycarbonyl; —C(═NOH)NH₂; —C(O)R¹⁷; —OR¹³; —NR11R₁₁ₐ; —NR¹²S(O)₂R^{12a}; optionally substituted heteroaryl; optionally substituted heterocycloalkyl; alkyl optionally substituted with 1, 2, 3, 4, or 5 R⁹ groups; alkenyl optionally substituted with one or two groups independently selected from carboxy and alkoxycarbonyl; or cycloalkyl optionally substituted with 1 or 2 groups independently selected from hydroxyalkyl, alkoxycarbonyl, carboxy, and —C(O)NR¹⁰R^{10a};
R⁷ and R⁸ are independently hydrogen, halo, haloalkyl, or alkyl;
each R⁹, when R⁹ is present, is independently cyano; hydroxy; halo; —C(O)H; —C(O)NR¹⁰R^{10a}; —C(O)OR¹⁰; —NR¹¹R₁₁ₐ; —NR¹²S(O)₂R^{12a}; —P(O)(OR¹⁶)₂; —OP(O)(OR¹⁶)₂; —OS(O)₂OH; —S(O)ₙR¹⁸; —C(═NOH)NH₂; optionally substituted heteroaryl; or heterocycloalkyl optionally substituted with 1, 2, or 3 groups independently selected from hydroxy, carboxy, alkoxycarbonyl, alkyl, hydroxyalkyl, and alkoxycarbonylamino;
R¹⁰ is hydrogen, alkyl, alkenyl, or alkynyl;
R^{10a} is hydrogen, alkyl, alkenyl, or alkynyl;
R^{10b} is hydrogen, alkyl, hydroxyalkyl, carboxyalkyl, haloalkyl, alkenyl, alkynyl, or alkyl substituted with one or two groups independently selected from —P(O)(OR¹⁶)₂, —OP(O)(OR¹⁶)₂, —OS(O)₂OH, and —OSi(alkyl)₃;
R¹¹ is hydrogen, alkyl, alkenyl, or alkynyl;
R^{11a} is hydrogen, alkyl, alkenyl, alkynyl, alkylsulfonyl, alkoxycarbonyl, carboxyalkyl, or hydroxyalkyl;
R¹² is hydrogen, alkyl, alkenyl, or alkynyl;
R^{12a} is alkyl, alkenyl, alkynyl, aminoalkyl, alkylaminoalkyl, or dialkylaminoalkyl;
R¹³ is alkenyl; alkyl optionally substituted with 1, 2, 3, or 4 groups independently selected from halo, hydroxy, alkoxy, alkylsulfanyl, alkylsulfonyl, cyano, —C(O)OR¹⁰, —OC(O)R^{10b}, —C(O)R^{10b}, —NR¹¹R^{11a}, —P(O)(OR¹⁶)₂, —OP(O)(OR¹⁶)₂, —OS(O)₂OH, —OSi(alkyl)₃, and heterocycloalkyl where the heterocycloalkyl is optionally substituted with one, two, or three groups independently selected from alkyl, carboxy, alkoxycarbonyl, alkoxycarbonylamino, and phenyl; or heterocycloalkyl optionally substituted with 1 or 2 groups independently selected from alkyl, carboxy, hydroxyalkyl, carboxyalkyl, and phenyl;
each R¹⁶ is independently hydrogen or alkyl;
R¹⁷ is amino, halo or alkyl substituted with one or two groups independently selected from carboxy or alkoxycarbonyl;
R¹⁸ is alkyl; and
n is 0, 1, or 2;
provided that when R⁵ is phenyl substituted with R⁶, R⁷, and R⁸ and
   a)

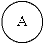

is furanyl and R⁶ is halo or cyano
b)

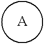

is thienyl and R⁶ is unsubstituted alkyl,

c)

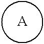

is oxadiazolyl, R⁶ is —OR¹³, and R¹³ is unsubstituted alkyl, or
d)

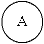

is oxazoyl, R⁶ is alkyl substituted with 3 R⁹, and each R⁹ is halo, then at least one of R⁷ and R⁸ is not hydrogen.

In a second aspect, the invention is directed to a pharmaceutical composition which comprises 1) a compound of Formula I or a single stereoisomer or mixture of isomers thereof, all optionally as a pharmaceutically acceptable salt thereof and 2) a pharmaceutically acceptable carrier, excipient, or diluent. In some embodiments, the pharmaceutically acceptable excipient is water, in which case the composition optionally comprises an additional pharmaceutically aceeptable excipient.

In a third aspect, the Invention provides a method for treating a disease, disorder, or syndrome which method comprises administering to a patient a therapeutically effective amount of a compound of Formula I or a single stereoisomer or mixture of isomers thereof, all optionally as a pharmaceutically acceptable salt or solvate thereof, or a pharmaceutical composition comprising a therapeutically effective amount of a compound of Formula I or a single stereoisomer or mixture of isomers thereof, all optionally as a pharmaceutically acceptable salt or solvate thereof, and a pharmaceutically acceptable carrier, excipient, or diluent. In some embodiments, the pharmaceutically acceptable excipient is water, in which case the composition optionally comprises an additional pharmaceutically aceeptable excipient.

In a fourth aspect, the Invention is directed to a method of making a Compound of Formula I, comprising
(a) reacting a compound of formula (g):

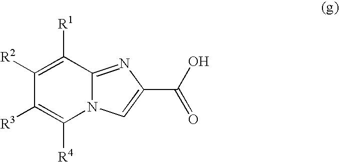

where R¹, R², R³, and R⁴ are as defined in the Summary of the Invention for a Compound of Formula I, with a reagent R⁵C(═NOH)NH₂ (j) where R⁵ is as defined in the Summary of the Invention for a Compound of Formula I; to yield a Compound of Formula I(a):

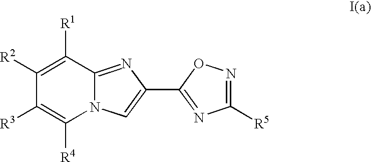

or
(b) reacting a compound of formula (k):

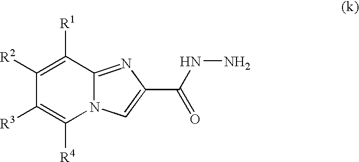

where R¹, R², R³, and R⁴ are as defined in the Summary of the Invention for a Compound of Formula I, with a reagent R⁵C(O)OH (m) where R⁵ is as defined in the Summary of the Invention for a Compound of Formula I, followed by treatment with EtSH to yield a Compound of Formula I(j):

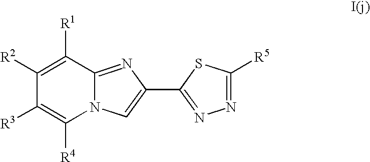

or
(c) reacting a compound of formula (g) as described above with a reagent of formula R⁵C(O)NHNH₂ (p) where R⁵ is as defined in the Summary of the Invention for a Compound of Formula I, to yield a Compound of Formula I(e):

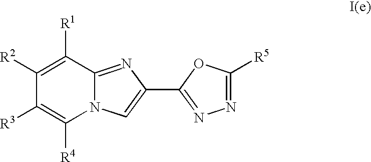

or
(d) reacting a compound of formula (q):

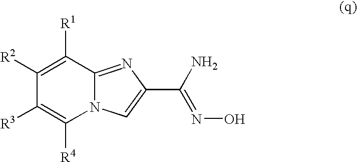

where R¹, R², R³, and R⁴ are as defined in the Summary of the Invention for a Compound of Formula I, with a reagent R⁵C(O)OH (r) where R⁵ is as defined in the Summary of the Invention for a Compound of Formula I, to yield a Compound of Formula I(c):

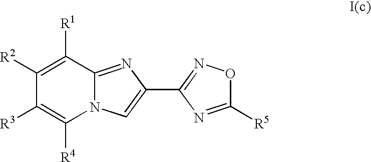

or
(e) reacting a compound of formula I(n):

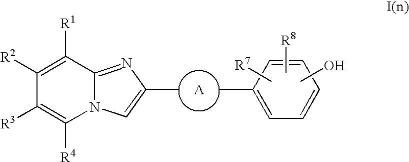

where R¹, R², R³, R⁴, R⁷, and R⁸ are as defined in the Summary of the Invention for a Compound of Formula I, with a reagent of formula R¹³X where X is halo and R¹³ is as defined in the Summary of the Invention for a Compound of Formula I, to yield a Compound of Formula I(p):

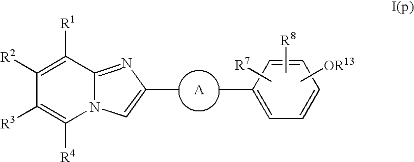

and
(f) optionally modifying any of R¹, R², R³, R⁴, and R⁵ and the substituents contained therein; and
(g) optionally further resolving individual isomers.

### DETAILED DESCRIPTION OF THE INVENTION

### Abbreviations and Definitions

The following abbreviations and terms have the indicated meanings throughout:

| Abbreviation | Meaning |
|---|---|
| br | broad |
| ° C. | degrees Celsius |
| CBZ | CarboBenZoxy = benzyloxycarbonyl |
| conc. | concentrated |
| d | doublet |
| dd | doublet of doublet |
| dt | doublet of triplet |
| dba | trans,trans-dibenzylideneacetone |
| DCM | dichloromethane |
| DIBAL | Diisobutylaluminium hydride |
| DMA | N,N-dimethylacetamide |
| DMF | N,N-dimethylformamide |
| DMSO | dimethyl sulfoxide |
| dppf | 1,1′-bis(diphenylphosphano)ferrocene |
| EDCI | 1-(3-dimethylaminopropyl)-3-ethylcarbodiimide |
| EI | Electron Impact ionization |
| EtOAc | ethyl acetate |
| EtOH | ethanol |
| g | gram(s) |
| h or hr | hour(s) |
| HOBt | N-hydroxybenzotriazole |
| HPLC | high pressure liquid chromatography |
| iPrOH | isopropanol |
| L | liter(s) |
| M | molar or molarity |
| m | Multiplet |
| MeOH | methanol |
| mg | milligram(s) |
| MHz | megahertz (frequency) |
| Min | minute(s) |
| mL | milliliter(s) |
| μL | microliter(s) |
| μM | Micromole(s) or micromolar |
| mM | Millimolar |
| mmol | millimole(s) |
| mol | mole(s) |
| MS | mass spectral analysis |
| MsCl | mesyl chloride |
| N | normal or normality |
| nM | Nanomolar |
| NMO | N-methylmorpholine-N-oxide |
| NMR | nuclear magnetic resonance spectroscopy |
| PhMe | toluene |
| q | Quartet |
| rt, RT | Room temperature |
| s | Singlet |
| t or tr | Triplet |
| TBAF | tetrabutylammonium fluoride |
| TBDMS | tert-Butyldimethylsilyl |
| TFA | trifluoroacetic acid |
| THF | tetrahydrofuran |
| TLC | thin layer chromatography |
| p-TsOH | p-toluenesulfonic acid |

The symbol “—” means a single bond, “═” means a double bond, “≡ means a triple bond, [symbol] means a single or double bond. The symbol [symbol] refers to a group on a double-bond as occupying either position on the terminus of a double bond to which the symbol is attached; that is, the geometry, E- or Z—, of the double bond is ambiguous. When a group is depicted removed from its parent formula, the [symbol] symbol will be used at the end of the bond which was theoretically cleaved in order to separate the group from its parent structural formula.

When chemical structures are depicted or described, unless explicitly stated otherwise, all carbons are assumed to have hydrogen substitution to conform to a valence of four. For example, in the structure on the left-hand side of the schematic below there are nine hydrogens implied. The nine hydrogens are depicted in the right-hand structure. Sometimes a particular atom in a structure is described in textual formula as having a hydrogen or hydrogens as substitution (expressly defined hydrogen), for example, —CH₂CH₂—. It is understood by one of ordinary skill in the art that the aforementioned descriptive techniques are common in the chemical arts to provide brevity and simplicity to description of otherwise complex structures.

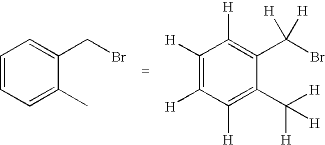

If a group “R” is depicted as “floating” on a ring system, as for example in the formula:

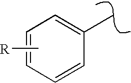

then, unless otherwise defined, a substituent “R” may reside on any atom of the ring system, assuming replacement of a depicted, implied, or expressly defined hydrogen from one of the ring atoms, so long as a stable structure is formed.

If a group “R” is depicted as floating on a fused or bridged ring, as for example in the formulae:

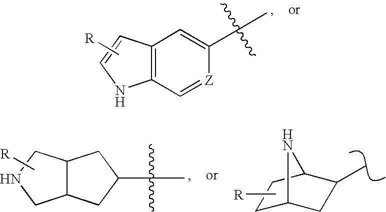

then, unless otherwise defined, a substituent “R” may reside on any atom of the fused or bridged ring, assuming replacement of a depicted hydrogen (for example the —NH— in the formula above), implied hydrogen (for example as in the formula above, where the hydrogens are not shown but understood to be present), or expressly defined hydrogen (for example where in the formula above, “Z” equals ═CH—) from one of the ring atoms, so long as a stable structure is formed. In the example depicted, the “R” group may reside on either the 5-membered or the 6-membered ring of the fused or bridged ring. In the formula depicted above, when y is 2 for example, then the two “R's” may reside on any two atoms of the ring system, again assuming each replaces a depicted, implied, or expressly defined hydrogen on the ring.

When a group “R” is depicted as existing on a ring system containing saturated carbons, as for example in the formula:

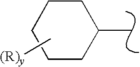

where, in this example, “y” can be more than one, assuming each replaces a currently depicted, implied, or expressly defined hydrogen on the ring; then, unless otherwise defined, where the resulting structure is stable, two “R's” may reside on the same carbon. A simple example is when R is a methyl group; there can exist a geminal dimethyl on a carbon of the depicted ring (an “annular” carbon). In another example, two R's on the same carbon, including that carbon, may form a ring, thus creating a spirocyclic ring (a “spirocyclyl” group) structure with the depicted ring as for example in the formula:

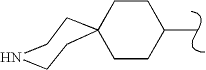

Within a particular substituent or term defined herein, as used throughout the specification, there may be two or more groups of the same type (e.g., two alkyl groups or two aryl groups). Unless specifically stated to the contrary, each of these groups can be the same or different from every other group of the same type. For example, “dialkylamino” is defined to mean an —NRR′ radical where R and R′ are each alkyl. In this example, each of the alkyls can be the same alkyl or they can be different.

“Acyl” means a —C(O)R radical where R is optionally substituted alkyl, optionally substituted alkenyl, cycloalkyl, cycloalkylalkyl, aryl, aralkyl, heteroaryl, heteroaralkyl, heterocycloalkyl, or heterocycloalkylalkyl, as defined herein, e.g., acetyl, trifluoromethylcarbonyl, or 2-methoxyethylcarbonyl, and the like.

“Acylamino” means a —NRR′ radical where R is hydrogen, hydroxy, alkyl, or alkoxy and R′ is acyl, as defined herein.

“Acyloxy” means an —OR radical where R is acyl, as defined herein, e.g. cyanomethylcarbonyloxy, and the like.

“Administration” and variants thereof (e.g., “administering” a compound of the invention) in reference to a compound of the invention means introducing the compound or a prodrug of the compound into the system of the animal in need of treatment. When a compound of the invention or prodrug thereof is provided in combination with one or more other active agents (e.g., surgery, radiation, and chemotherapy, etc.), “administration” and its variants are each understood to include concurrent and sequential introduction of the compound or prodrug thereof and other agents.

“Alkenyl” and “C₂₋₆-alkenyl” mean a linear monovalent hydrocarbon radical of two to six carbon atoms or a branched monovalent hydrocarbon radical of three to six carbon atoms which radical contains at least one double bond, e.g., ethenyl, propenyl, 1-but-3-enyl, and 1-pent-3-enyl, and the like.

“Alkoxy” and “C₁₋₆-alkoxy” mean an —OR group where R is alkyl group as defined herein. Examples include methoxy, ethoxy, propoxy, isopropoxy, and the like.

“Alkoxyalkyl” and “C₁₋₆-alkoxy-C₁₋₆-alkyl” mean an alkyl group, as defined herein, substituted with at least one, specifically one, two, or three, alkoxy groups as defined herein. Representative examples include methoxymethyl and the like.

“Alkoxycarbonyl” and “C₁₋₆-alkoxycarbonyl” mean a —C(O)R group where R is alkoxy, as defined herein.

“Alkoxycarbonylalkyl” and “C₁₋₆-alkoxycarbonyl-C₁₋₆-alkyl mean an alkyl substituted with one or two alkoxycarbonyl groups as defined herein.

“Alkoxycarbonylamino” and “C₁₋₆-alkoxycarbonylamino” mean an —NHR group where R is alkoxycarbonyl as defined herein.

“Alkyl” and “C₁₋₆-alkyl” mean a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), or pentyl (including all isomeric forms), and the like.

“Alkylamino” and “C₁₋₆-alkylamino” mean an —NHR group where R is alkyl as defined herein.

“Alkylaminoalkyl” and “C₁₋₆-alkylamino-C₁₋₆-alkyl” mean an alkyl group substituted with one or two alkylamino groups, as defined herein.

“Alkylaminoalkyloxy” and “C₁₋₆-alkylamino-C₁₋₆-alkyloxy” mean an —OR group where R is alkylaminoalkyl, as defined herein.

“Alkylcarbonyl” and “C₁₋₆-alkylcarbonyl” mean a —C(O)R group where R is alkyl, as defined herein.

“Alkylsulfanyl” and “C₁₋₆-alkylsulfanyl” means an —SR group where R is alkyl as defined herein.

“Alkylsulfonyl” and “C₁₋₆-alkylsulfonyl” means an —S(O)₂R group where R is alkyl, as defined herein, e.g. methylsulfonyl, isopropylsulfonyl.

“Alkylsulfonylamino” and “C₁₋₆-alkylsulfonylamino” mean an —NRS(O)₂R′ group where R is hydrogen or alkyl, as defined herein, and R′ is alkyl, as defined herein.

“Alkynyl” and “C₂₋₆-alkynyl” mean a linear monovalent hydrocarbon radical of two to six carbon atoms or a branched monovalent hydrocarbon radical of three to 6 carbon atoms which radical contains at least one triple bond, e.g., ethynyl, propynyl, butynyl, pentyn-2-yl and the like.

“Amino” means —NH₂.

“Aminoalkyl” and “amino-C₁₋₆-alkyl” mean an alkyl group substiuted with at least one, specifically one, two or three, amino groups.

“Aminoalkyloxy” and “amino-C₁₋₆-alkyloxy” mean an —OR group where R is aminoalkyl, as defined herein.

“Aminocarbonyl” means a —C(O)NH₂ group.

“Alkylaminocarbonyl” means a —C(O)NHR group where R is alkyl as defined herein.

“Aryl” means a monovalent six- to fourteen-membered, mono- or bi-carbocyclic ring, wherein the monocyclic ring is aromatic and at least one of the rings in the bicyclic ring is aromatic. Unless stated otherwise, the valency of the group may be located on any atom of any ring within the radical, valency rules permitting. Representative examples include phenyl, naphthyl, and indanyl, and the like.

“Arylalkyl” and “aryl-C₁₋₆-alkyl” mean an alkyl radical, as defined herein, substituted with one or two aryl groups, as defined herein, e.g., benzyl and phenethyl, and the like.

“Carboxy” means a —C(O)OH group.

“Carboxyalkyl” and “carboxy-C₁₋₆-alkyl” means an alkyl group, as defined herein, substituted with at least one, specifically one, two, or three, —C(O)OH group(s).

“Cycloalkyl” means a monocyclic or fused, bridged, or spirocyclic bicyclic monovalent hydrocarbon radical of three to ten carbon ring atoms and where the ring(s) is saturated or partially unsaturated (but not aromatic). Unless stated otherwise, the valency of the group may be located on any atom of any ring within the radical, valency rules permitting. One or two ring carbon atoms are optionally substituted with ═O, ═S, or ═NH, to form a —C(O)—, —C(S)—, or —C(═NH)— group, respectively. More specifically, the term cycloalkyl includes, but is not limited to, cyclopropyl, cyclobutyl, cyclopentyl, cyclohexyl, cyclohexyl, or cyclohex-3-enyl, and the like.

“Cycloalkylalkyl” and “cycloalkyl-C₁₋₆-alkyl” mean an alkyl group substituted with at least one, specifically one or two, cycloalkyl group(s) as defined herein.

“Dialkylamino” and “di-(C₁₋₆-alkyl)amino” mean a —NRR′ radical where R and R′ are independently alkyl as defined herein, or an N-oxide derivative, or a protected derivative thereof, e.g., dimethylamino, diethylamino, N,N-methylpropylamino or N,N-methylethylamino, and the like.

“Dialkylaminoalkyl” and “di-(C₁₋₆-alkyl)amino-C₁₋₆-alkyl” mean an alkyl group substituted with one or two dialkylamino groups, as defined herein.

“Dialkylaminoalkyloxy” and “di-(C₁₋₆-alkyl)amino-C₁₋₆-alkyloxy” mean an —OR group where R is dialkylaminoalkyl, as defined herein. Representative examples include 2-(N,N-diethylamino)-ethyloxy, and the like.

“Dialkylaminocarbonyl” and “di-(C₁₋₆-alkyl)aminocarbonyl” mean a —C(O)NRR′ group where R and R′ are alkyl as defined herein.

“Halogen” or “halo” refers to fluorine, chlorine, bromine and iodine.

“Haloalkoxy” and “halo-C₁₋₆-alkoxy” means an —OR′ group where R′ is haloalkyl as defined herein, e.g., trifluoromethoxy or 2,2,2-trifluoroethoxy, and the like.

“Haloalkyl” and “halo-C₁₋₆-alkyl” mean an alkyl group substituted with one or more halogens, specifically one to five halo atoms, e.g., trifluoromethyl, 2-chloroethyl, and 2,2-difluoroethyl, and the like.

“Heteroaryl” means a monocyclic, fused bicyclic, or fused tricyclic, monovalent radical of 5 to 14 ring atoms containing one or more, specifically one, two, three, or four ring heteroatoms independently selected from —O—, —S(O)ₙ— (n is 0, 1, or 2), —N—, —N(R^{x})—, and the remaining ring atoms being carbon, wherein the monocyclic ring is aromatic and wherein at least one of the fused rings of a bicyclic or tricyclic radical is aromatic. One or two ring carbon atoms of any nonaromatic rings comprising a bicyclic or tricyclic radical may be substitued with ═O, ═S, or ═NH to form a —C(O)—, —C(S)—, or —C(═NH)— group, respectively. R^{x} is hydrogen, alkyl, hydroxy, alkoxy, acyl, or alkylsulfonyl. Fused bicyclic radical includes bridged ring systems. Unless stated otherwise, the valency may be located on any atom of any ring of the heteroaryl group, valency rules permitting. When the point of valency is located on the nitrogen, R^{x} is absent. More specifically, the term heteroaryl includes, but is not limited to, 1,2,4-triazolyl, 1,3,5-triazolyl, phthalimidyl, pyridinyl, pyrrolyl, imidazolyl, thienyl, furanyl, indolyl, 2,3-dihydro-1H-indolyl (including, for example, 2,3-dihydro-1H-indol-2-yl or 2,3-dihydro-1H-indol-5-yl, like), isoindolyl, indolinyl, isoindolinyl, benzimidazolyl, benzodioxol-4-yl, benzofuranyl, cinnolinyl, indolizinyl, naphthyridin-3-yl, phthalazin-3-yl, phthalazin-4-yl, pteridinyl, purinyl, quinazolinyl, quinoxalinyl, tetrazoyl, pyrazolyl, pyrazinyl, pyrimidinyl, pyridazinyl, oxazolyl, isooxazolyl, oxadiazolyl, benzoxazolyl, quinolinyl, isoquinolinyl, tetrahydroisoquinolinyl (including, for example, tetrahydroisoquinolin-4-yl or tetrahydroisoquinolin-6-yl, and the like), pyrrolo[3,2-c]pyridinyl (including, for example, pyrrolo[3,2-c]pyridin-2-yl or pyrrolo[3,2-c]pyridin-7-yl, and the like), benzopyranyl, thiazolyl, isothiazolyl, thiadiazolyl, benzothiazolyl, benzothienyl, and the derivatives thereof, or N-oxide or a protected derivative thereof.

“Heteroarylene” means a divalent heteroaryl group as otherwise defined herein.

“Heteroatom” refers to O, S, N, and P.

“Heterocycloalkyl” means a saturated or partially unsaturated (but not aromatic) monovalent monocyclic group of 3 to 8 ring atoms or a saturated or partially unsaturated (but not aromatic) monovalent fused, bridged, or spirocyclic bicyclic group of 5 to 12 ring atoms in which one or more, specifically one, two, three, or four ring heteroatoms independently selected from O, S(O)ₙ (n is 0, 1, or 2), N, N(R^{y}) (where R^{y} is hydrogen, alkyl, hydroxy, alkoxy, acyl, or alkylsulfonyl), and P, the remaining ring atoms being carbon. One or two ring carbon atoms may be substituted with ═O, ═S, or ═NH to form a —C(O)—, —C(S)—, or —C(═NH)— group, respectively. One or two ring phosphorous atoms may be substituted with a ═O and alkoxy to form a —P(O)(alkoxy)- group. Unless otherwise stated, the valency of the group may be located on any atom of any ring within the radical, valency rules permitting. When the point of valency is located on a nitrogen atom, Ry is absent. More specifically the term heterocycloalkyl includes, but is not limited to, azetidinyl, pyrrolidinyl, 2-oxopyrrolidinyl, 2,5-dihydro-1H-pyrrolyl, piperidinyl, 4-piperidonyl, morpholinyl, piperazinyl, 2-oxopiperazinyl, tetrahydropyranyl, 2-oxopiperidinyl, thiomorpholinyl, thiamorpholinyl, perhydroazepinyl, pyrazolidinyl, imidazolinyl, imidazolidinyl, dihydropyridinyl, tetrahydropyridinyl, oxazolinyl, oxazolidinyl, 2-oxo-1,3-oxazolidinyl, isoxazolidinyl, thiazolinyl, thiazolidinyl, quinuclidinyl, isothiazolidinyl, octahydroindolyl, octahydroisoindolyl, decahydroisoquinolyl, tetrahydrofuryl, tetrahydropyranyl, and 2-tent-butoxy-2-oxo-1,3,2-dioxaphospholanyl, and derivatives thereof and N-oxide or a protected derivative thereof.

“Heterocycloalkylalkyl” and “heterocycloalkyl-C₁₋₆-alkyl” mean an alkyl radical, as defined herein, substituted with one or two heterocycloalkyl groups, as defined herein, e.g., morpholinylmethyl, N-pyrrolidinylethyl, and 3-(N-azetidinyl)propyl, and the like.

“Hydroxyalkyl” and “hydroxy-C₁₋₆-alkyl” mean an alkyl group substituted with at least one, in another example with one, two, or three, hydroxy groups.

“Optional” or “optionally” means that the subsequently described event or circumstance may or may not occur, and that the description includes instances where said event or circumstance occurs and instances in which it does not. One of ordinary skill in the art would understand that with respect to any molecule described as containing one or more optional substituents, only sterically practical and/or synthetically feasible compounds are meant to be included. “Optionally substituted” refers to all subsequent modifiers in a term. So, for example, in the term “optionally substituted arylC₁₋₈ alkyl,” optional substitution may occur on both the “C₁₋₈ alkyl” portion and the “aryl” portion of the molecule may or may not be substituted.

“Optionally substituted alkyl” means an alkyl radical, as defined herein, optionally substituted with one or more group(s), specifically one, two, three, four, or five groups, independently selected from alkylcarbonyl, alkenylcarbonyl, cycloalkylcarbonyl, alkylcarbonyloxy, alkenylcarbonyloxy, amino, alkylamino, dialkylamino, aminocarbonyl, alkylaminocarbonyl, dialkylaminocarbonyl, cyano, cyanoalkylaminocarbonyl, alkoxy, alkenyloxy, hydroxy, hydroxyalkoxy, halo, carboxy, alkylcarbonylamino, alkylcarbonyloxy, alkyl-S(O)₀₋₂—, alkenyl-S(O)₀₋₂—, aminosulfonyl, alkylaminosulfonyl, dialkylaminosulfonyl, alkylsulfonyl-NR^{c}— (where R^{c} is hydrogen, alkyl, optionally substituted alkenyl, hydroxy, alkoxy, alkenyloxy, or cyanoalkyl), alkylaminocarbonyloxy, dialkylaminocarbonyloxy, alkylaminoalkyloxy, dialkylaminoalkyloxy, alkoxycarbonyl, alkenyloxycarbonyl, alkoxycarbonylamino, alkylaminocarbonylamino, dialkylaminocarbonylamino, alkoxyalkyloxy, and —C(O)NR^{a}R^{b} (where R^{a} and R^{b} are independently hydrogen, alkyl, optionally substituted alkenyl, hydroxy, alkoxy, alkenyloxy, or cyanoalkyl).

“Optionally substituted alkenyl” means an alkyl radical, as defined herein, optionally substituted with one or more group(s), specifically one, two, three, four, or five groups, independently selected from alkylcarbonyl, alkenylcarbonyl, cycloalkylcarbonyl, alkylcarbonyloxy, alkenylcarbonyloxy, amino, alkylamino, dialkylamino, aminocarbonyl, alkylaminocarbonyl, dialkylaminocarbonyl, cyano, cyanoalkylaminocarbonyl, alkoxy, alkenyloxy, hydroxy, hydroxyalkoxy, halo, carboxy, alkylcarbonylamino, alkylcarbonyloxy, alkyl-S(O)₀₋₂—, alkenyl-S(O)₀₋₂—, aminosulfonyl, alkylaminosulfonyl, dialkylaminosulfonyl, alkylsulfonyl-NR^{c}— (where R^{c} is hydrogen, alkyl, alkenyl, hydroxy, alkoxy, alkenyloxy, or cyanoalkyl), alkylaminocarbonyloxy, dialkylaminocarbonyloxy, alkylaminoalkyloxy, dialkylaminoalkyloxy, alkoxycarbonyl, alkenyloxycarbonyl, alkoxycarbonylamino, alkylaminocarbonylamino, dialkylaminocarbonylamino, alkoxyalkyloxy, and —C(O)NR^{a}R^{b} (where R^{a} and R^{b} are independently hydrogen, alkyl, alkenyl, hydroxy, alkoxy, alkenyloxy, or cyanoalkyl).

“Optionally substituted heteroaryl” means a heteroaryl group optionally substituted with one, two, or three substituents independently selected from acyl, acylamino, acyloxy, optionally substituted alkyl, optionally substituted alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarbonyl, carboxy, cyano, alkylthio, alkylsulfinyl, alkylsulfonyl, aminosulfonyl, alkylaminosulfonyl, dialkylaminosulfonyl, alkylsulfonylamino, aminoalkoxy, alkylaminoalkoxy, and dialkylaminoalkoxy. Within the optional substituents on “heteroaryl”, the alkyl and alkenyl, either alone or as part of another group (including, for example, the alkyl in alkoxycarbonyl), are independently optionally substituted with one, two, three, four, or five halo.

“Optionally substituted heterocycloalkyl” means a heterocycloalkyl group, as defined herein, optionally substituted with one, two, or three substituents independently selected from phenyl, acyl, acylamino, acyloxy, optionally substituted alkyl, optionally substituted alkenyl, alkoxy, alkenyloxy, halo, hydroxy, alkoxycarbonyl, alkenyloxycarbonyl, alkoxycarbonylamino, amino, alkylamino, dialkylamino, nitro, aminocarbonyl, alkylaminocarbonyl, dialkylaminocarbonyl, carboxy, cyano, alkylthio, alkylsulfinyl, alkylsulfonyl, aminosulfonyl, alkylaminosulfonyl, dialkylaminosulfonyl, alkylsulfonylamino, and aminoalkoxy. Within the optional substituents on “heterocycloalkyl”, the alkyl and alkenyl, either alone or as part of another group (including, for example, the alkyl in alkoxycarbonyl), are independently optionally substituted with one, two, three, four, or five halo.

“Optionally substituted phenoxy” means an —OR group where R is optionally substituted phenyl as defined herein.

“Optionally substituted phenyl” means a phenyl group optionally substituted with one, two, or three substituents independently selected from acyl, acylamino, acyloxy, optionally substituted alkyl, optionally substituted alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarbonyl, carboxy, cyano, alkylthio, alkylsulfinyl, alkylsulfonyl, aminosulfonyl, alkylaminosulfonyl, dialkylaminosulfonyl, alkylsulfonylamino, aminoalkoxy, or aryl is pentafluorophenyl. Within the optional substituents on the “phenyl”, the alkyl and alkenyl, either alone or as part of another group (including, for example, the alkyl in alkoxycarbonyl), are independently optionally substituted with one, two, three, four, or five halo.

“Yield” for each of the reactions described herein is expressed as a percentage of the theoretical yield.

“Metabolite” refers to the break-down or end product of a compound of the invention or its salt produced by metabolism or biotransformation in the animal or human body; for example, biotransformation to a more polar molecule such as by oxidation, reduction, or hydrolysis, or to a conjugate (see Goodman and Gilman, “The Pharmacological Basis of Therapeutics” 8.sup.th Ed., Pergamon Press, Gilman et al. (eds), 1990 for a discussion of biotransformation). As used herein, the metabolite of a compound of the invention or its salt may be the biologically active form of the compound in the body. In one example, a prodrug may be used such that the biologically active form, a metabolite, is released in vivo. In another example, a biologically active metabolite is discovered serendipitously, that is, no prodrug design per se was undertaken. An assay for activity of a metabolite of a compound of the present invention is known to one of skill in the art in light of the present disclosure.

“Patient” for the purposes of the present invention includes humans and other animals, particularly mammals, and other organisms. Thus the methods are applicable to both human therapy and veterinary applications. In a specific embodiment the patient is a mammal, and in a more specific embodiment the patient is human.

A “pharmaceutically acceptable salt” of a compound of the invention means a salt that is pharmaceutically acceptable and that possesses the desired pharmacological activity of the parent compound. It is understood that the pharmaceutically acceptable salts are non-toxic. Additional information on suitable pharmaceutically acceptable salts can be found in Remington's Pharmaceutical Sciences, 17^{th} ed., Mack Publishing Company, Easton, Pa., 1985, which is incorporated herein by reference or S. M. Berge, et al., “Pharmaceutical Salts,” J. Pharm. Sci., 1977;66:1-19 both of which are incorporated herein by reference.

Examples of pharmaceutically acceptable acid addition salts include those formed with inorganic acids such as hydrochloric acid, hydrobromic acid, sulfuric acid, nitric acid, phosphoric acid, and the like; as well as organic acids such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid and the like.

Examples of a pharmaceutically acceptable base addition salts include those formed when an acidic proton present in the parent compound is replaced by a metal ion, such as sodium, potassium, lithium, ammonium, calcium, magnesium, iron, zinc, copper, manganese, aluminum salts and the like. Specific salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins. Examples of organic bases include isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, tromethamine, N-methylglucamine, polyamine resins, and the like. Exemplary organic bases are isopropylamine, diethylamine, ethanolamine, trimethylamine, dicyclohexylamine, choline, and caffeine.“Platin(s),” and “platin-containing agent(s)” include, for example, cisplatin, carboplatin, and oxaliplatin.

“Prodrug” refers to compounds that are transformed (typically rapidly) in vivo to yield the parent compound of the above formulae, for example, by hydrolysis in blood. Common examples include, but are not limited to, ester and amide forms of a compound having an active form bearing a carboxylic acid moiety. Examples of pharmaceutically acceptable esters of the compounds of this invention include, but are not limited to, alkyl esters (for example with between about one and about six carbons) the alkyl group is a straight or branched chain. Acceptable esters also include cycloalkyl esters and arylalkyl esters such as, but not limited to benzyl. Examples of pharmaceutically acceptable amides of the compounds of this invention include, but are not limited to, primary amides, and secondary and tertiary alkyl amides (for example with between about one and about six carbons). Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

“Therapeutically effective amount” is an amount of a compound of the invention, that when administered to a patient, ameliorates a symptom of the disease. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art having regard to their knowledge and to this disclosure.

“Treating” or “treatment” of a disease, disorder, or syndrome, as used herein, includes (i) preventing the disease, disorder, or syndrome from occurring in a human, i.e. causing the clinical symptoms of the disease, disorder, or syndrome not to develop in an animal that may be exposed to or predisposed to the disease, disorder, or syndrome but does not yet experience or display symptoms of the disease, disorder, or syndrome; (ii) inhibiting the disease, disorder, or syndrome, i.e., arresting its development; and (iii) relieving the disease, disorder, or syndrome, i.e., causing regression of the disease, disorder, or syndrome. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

### Embodiments of the Invention

The following paragraphs present a number of embodiments of compounds of the invention. In each instance the embodiment includes both the recited compounds, as well as a single stereoisomer or mixture of stereoisomers thereof, as well as a pharmaceutically acceptable salt thereof.

In the following embodiments, whenever the conditions of the following proviso is applicable in whole or in part, the scope of the embodiment is limited according to the proviso: “provided that when R⁵ is phenyl substituted with R⁶, R⁷, and R⁸ and
a)

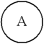

is is furanyl and R⁶ is halo or cyano
b)

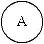

is thienyl and R⁶ is unsubstituted alkyl,
c)

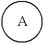

is oxadiazolyl, R⁶ is —OR¹³, and R¹³ is unsubstituted alkyl, or
d)

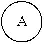

is oxazoyl, R⁶ is alkyl substituted with 3 R⁹, and each R⁹ is halo,
then at least one of R⁷ and R⁸ is not hydrogen.”

One embodiment of the Invention (1) is directed to a Compound of Formula I wherein

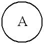

is oxadiazolyl or thiadiazolyl and all other groups are as defined in the Summary of the Invention.

One embodiment (A) of the Invention is directed to a Compound of Formula I wherein

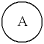

is oxadiazolyl and all other groups are as defined in the Summary of the Invention.

Another embodiment (B) of the Invention is directed to a Compound of Formula I wherein

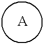

is thiadiazolyl and all other groups are as defined in the Summary of the Invention.

Another embodiment (C) of the Invention is directed to a Compound of Formula I wherein R¹ is hydrogen, halo, cyano, alkoxy, amino, alkylamino, or dialkylamino; and all other groups are as defined in the Summary of the Invention or as defined in any of embodiments 1, A, and B. In another embodiment is directed to a Compound of Formula I wherein R¹ is hydrogen, halo or cyano; and all other groups are as defined in the Summary of the Invention or as defined in any of embodiments 1, A, and B. In another embodiment, the Invention is directed to a Compound of Formula I wherein R¹ is halo; and all other groups are as defined in the Summary of the Invention or as defined in any of embodiments 1, A, and B.

Another embodiment (D) of the Invention is directed to a Compound of Formula I wherein R² is hydrogen, methyl, or methoxy; and all other groups are as defined in the Summary of the Invention or as defined in any of embodiments 1, A, B, and C. In another embodiment, the Invention is directed to a Compound of Formula I wherein R² is hydrogen; and all other groups are as defined in the Summary of the Invention or as defined in any of embodiments 1, A, B, and C.

Another embodiment (E) of the Invention is directed to a Compound of Formula I wherein R³ is hydrogen, alkyl, alkylsulfonyl, halo, haloalkyl, alkoxy, optionally substituted phenoxy, cyano, alkylsulfonylamino, or nitro; and all other groups are as defined in the Summary of the Invention or as defined in any of embodiments 1, A, B, C, and D. In another embodiment, the Invention is directed to a Compound of Formula I wherein R³ is hydrogen, alkyl, halo, or haloalkyl; and all other groups are as defined in the Summary of the Invention or as defined in any of embodiments 1, A, B, C, and D. In another embodiment, the Invention is directed to a Compound of Formula I wherein R³ is hydrogen; and all other groups are as defined in the Summary of the Invention or as defined in any of embodiments 1, A, B, C, and D. In another embodiment, the Invention is directed to a Compound of Formula I wherein R³ is alkyl; and all other groups are as defined in the Summary of the Invention or as defined in any of embodiments 1, A, B, C, and D. In another embodiment, the Invention is directed to a Compound of Formula I wherein R³ is alkylsulfonyl; and all other groups are as defined in the Summary of the Invention or as defined in any of embodiments 1, A, B, C, and D. In another embodiment, the Invention is directed to a Compound of Formula I wherein R³ is halo; and all other groups are as defined in the Summary of the Invention or as defined in any of embodiments 1, A, B, C, and D. In another embodiment, the Invention is directed to a Compound of Formula I wherein R³ is haloalkyl; and all other groups are as defined in the Summary of the Invention or as defined in any of embodiments 1, A, B, C, and D. In another embodiment, the Invention is directed to a Compound of Formula I wherein R³ is trifluoromethyl; and all other groups are as defined in the Summary of the Invention or as defined in any of embodiments 1, A, B, C, and D. In another embodiment, the Invention is directed to a Compound of Formula I wherein R³ is alkoxy; and all other groups are as defined in the Summary of the Invention or as defined in any of embodiments 1, A, B, C, and D. In another embodiment, the Invention is directed to a Compound of Formula I wherein R³ is optionally substituted phenoxy; and all other groups are as defined in the Summary of the Invention or as defined in any of embodiments 1, A, B, C, and D. In another embodiment, the Invention is directed to a Compound of Formula I wherein R³ is cyano; and all other groups are as defined in the Summary of the Invention or as defined in any of embodiments 1, A, B, C, and D. In another embodiment, the Invention is directed to a Compound of Formula I wherein R³ is alkylsulfonylamino; and all other groups are as defined in the Summary of the Invention or as defined in any of embodiments 1, A, B, C, and D. In another embodiment, the Invention is directed to a Compound of Formula I wherein R³ is nitro; and all other groups are as defined in the Summary of the Invention or as defined in any of embodiments 1, A, B, C, and D.

Another embodiment (F) of the Invention is directed to a Compound of Formula I wherein R⁴ is hydrogen or alkyl; and all other groups are as defined in the Summary of the Invention or as defined in any of embodiments 1, A, B, C, D, and E. In another embodiment, the Invention is directed to a Compound of Formula I wherein R⁴ is hydrogen or methyl; and all other groups are as defined in the Summary of the Invention or as defined in any of embodiments 1, A, B, C, D, and E. In another embodiment, the Invention is directed to a Compound of Formula I wherein R⁴ is hydrogen; and all other groups are as defined in the Summary of the Invention or as defined in any of embodiments 1, A, B, C, D, and E.

Another embodiment (G) of the Invention is directed to a Compound of Formula I where at least one of R¹, R², R³, and R⁴ is not hydrogen and R¹, R², R³, and R⁴ are otherwise as define as well as all other groups, in the Summary of the Invention or in any of embodiments 1, A, B, C, D, E, and F. In another embodiment, the Invention is directed to a Compound of Formula I wherein R⁴ is hydrogen, at least one of R¹, R², and R³ is not hydrogen and R¹, R², and R³ are otherwise as defined, as well as all other groups, in the Summary of the Invention or in any of embodiments 1, A, B, C, D, E, and F. In another embodiment, the Invention is directed to a Compound of Formula I wherein R² and R⁴ are hydrogen, at least one of R¹ and R³ is not hydrogen, and R¹ and R³ are otherwise as defined, as well as all other groups, in the Summary of the Invention or in any of embodiments 1, A, B, C, D, E, and F.

Another embodiment (G1) of the Invention is directed to a Compound of Formula I where R² and R⁴ are hydrogen; R¹ is halo, cyano, alkoxy, amino, alkylamino, or dialkylamino; R³ is alkyl, alkylsulfonyl, halo, haloalkyl, alkoxy, optionally substituted phenoxy, cyano, alkylsulfonylamino, or nitro; and all other groups are as efined in the Summary of the Invention or as defined in any of embodiments 1, A, and B. In another the Compound of Formula I is that where R² and R⁴ are hydrogen; R¹ is halo or cyano; R³ is alkyl, halo, haloalkyl, or cyano; and all other groups are as defined in the Summary of the Invention or as defined in any of embodiments 1, A, and B. In another the Compound of Formula I is that where R² and R⁴ are hydrogen; R¹ is halo; and R³ is haloalkyl; and all other groups are as defined in the Summary of the Invention or as defined in any of embodiments 1, A, and B. In another the Compound of Formula I is that where R² and R⁴ are hydrogen; R¹ is halo; and R³ is trifluoromethyl; and all other groups are as defined in the Summary of the Invention or as defined in any of embodiments 1, A, and B.

Another embodiment (H) of the Invention is directed to a Compound of Formula I where R⁵ is heteroaryl optionally substituted with one or two R¹⁵ groups independently selected from alkyl; carboxy; haloalkyl; carboxyalkyl; alkoxycarbonylalkyl; and alkyl substituted with one —C(O)NR¹⁴R^{14a} group where R¹⁴ is hydrogen, alkyl, haloalkyl, or hydroxyalkyl and R^{14a} is hydrogen, alkyl, haloalkyl, hydroxyalkyl, or alkyl substituted with —O—Si(alkyl)₃; provided that when the R⁵ heteroaryl is pyridinyl or thienyl, then the pyridinyl and thienyl is substituted with one R¹⁵ and optionally substituted with an independently selected second R¹⁵; and all other groups are as defined in the Summary of the Invention or as defined in any of embodiments 1, A, B, C, D, E, F, G, and G1. In another embodiment, the Compond of Formula I is that where R⁵ is heteroaryl optionally substituted with one or two R¹⁵ groups independently selected from alkyl; carboxy; haloalkyl; carboxyalkyl; and alkyl substituted with one —C(O)NR¹⁴R^{14a} group where R¹⁴ and R^{14a} are independently hydrogen, alkyl, haloalkyl, or hydroxyalkyl; provided that when the R⁵ heteroaryl is pyridinyl or thienyl, then the pyridinyl or thienyl is substituted with one R¹⁵ and optionally substituted with an independently selected second R¹⁵; and all other groups are as defined in the Summary of the Invention or as defined in any of embodiments 1, A, B, C, D, E, F, G, and G1. In another embodiment, the Invention is directed to a Compound of Formula I wherein R⁵ is pyridinyl substituted with one R¹⁵ and the R¹⁵ is haloalkyl; and all other groups are as defined in the Summary of the Invention or as defined in any of embodiments 1, A, B, C, D, E, F, G, and G1. In another embodiment, the Invention is directed to a Compound of Formula I wherein R⁵ is unsubstituted benzimidazolyl; and all other groups are as defined in the Summary of the Invention or as defined in any of embodiments 1, A, B, C, D, E, F, G, and G1. In another embodiment, the Invention is directed to a Compound of Formula I wherein R⁵ is benzofuranyl optionally substituted with one R¹⁵ and the R¹⁵ is carboxy; and all other groups are as defined in the Summary of the Invention or as defined in any of embodiments 1, A, B, C, D, E, F, G, and G1.

In another embodiment (H1), the Invention is directed to a Compound of Formula I wherein R⁵ is indolyl optionally substituted with one R¹⁵ group selected from carboxy, carboxyalkyl, and alkyl substituted with one —C(O)NR¹⁴R^{14a}; and all other groups are as defined in the Summary of the Invention or as defined in any of embodiments 1, A, B, C, D, E, F, G, and G1. In another embodiment, the Invention is directed to a Compound of Formula I wherein R⁵ is indolyl optionally substituted with one R¹⁵ group selected from carboxyalkyl, and alkyl substituted with one —C(O)NR¹⁴R^{14a}; and all other groups are as defined in the Summary of the Invention or as defined in any of embodiments 1, A, B, C, D, E, F, G, and G1. In another embodiment, the Invention is directed to a Compound of Formula I wherein R⁵ is indol-4-yl optionally substituted with one R¹⁵ and the R¹⁵ is —C(O)NR¹⁴R^{14a}; and all other groups are as defined in the Summary of the Invention or as defined in any of embodiments 1, A, B, C, D, E, F, G, and G1.

Another embodiment (J) of the Invention is directed to a Compound of Formula I where R⁵ is phenyl substituted with R⁶, R⁷, and R⁸ and R⁶, R⁷, and R⁸ are as defined in the Summary of the Invention; and all other groups are as defined in the Summary of the Invention or as defined in any of embodiments 1, A, B, C, D, E, F, G, and G1.

Another embodiment (J1) of the Invention is directed to a Compound of Formula I where R⁵ is according to formula (a)

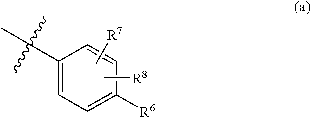

and R⁶, R⁷, and R⁸ and all other groups are as defined in the Summary of the Invention or as defined in any of embodiments 1, A, B, C, D, E, F, G, and G1. In another embodiment, the Compound of Formula I is that where R⁵ is according to formula (a) and at least one of R⁷ and R⁸ is not hydrogen and R⁷ and R⁸ are otherwise as defined, as well as all other groups, in the Summary of the Invention or in any of the embodiments 1, A, B, C, D, E, F, G, and G1. In another embodiment, the Compound of Formula I is that where R⁵ is according to formula (a) and both R⁷ and R⁸ are not hydrogen and R⁷ and R⁸ are otherwise as defined, as well as all other groups, in the Summary of the Invention or in any of the embodiments 1, A, B, C, D, E, F, G, and G1.

Another embodiment (J2) of the Invention is directed to a Compound of Formula I where R⁵ is according to formula (b)

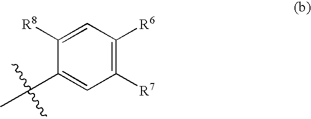

and R⁶, R⁷, and R⁸ where R⁷ and R⁸ are not hydrogen and R⁷ and R⁸ are otherwise as defined, as well as all other groups, in the Summary of the Invention or as defined in any of embodiments 1, A, B, C, D, E, F, G, and G1.

Another embodiment (J3) of the Invention is directed to a Compound of Formula I where R⁵ is according to formula (c)

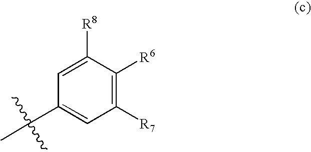

and R⁶, R⁷, and R⁸ where R⁷ and R⁸ are not hydrogen and R⁷ and R⁸ are otherwise as defined, as well as all other groups, in the Summary of the Invention or as defined in any of embodiments 1, A, B, C, D, E, F, G, and G1.

Another embodiment (J4) of the Invention is directed to a Compound of Formula I where R⁵ is according to formula (d)

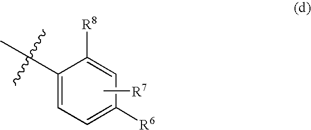

and R⁶, R⁷, and R⁸ where R⁸ is not hydrogen and R⁷ and R⁸ are otherwise as defined, as well as all other groups, in the Summary of the Invention or as defined in any of embodiments 1, A, B, C, D, E, F, G, and G1.

Another embodiment (K) of the Invention is directed to a Compound of Formula I where R⁶ is halo; hydroxy; cyano; —C(O)H; carboxy; alkoxycarbonyl; —C(═NOH)NH₂; —C(O)R¹⁷; —OR¹³; —NR11R^{11a}; —NR¹²S(O)₂—R^{12a}; optionally substituted heteroaryl; optionally substituted heterocycloalkyl; alkyl optionally substituted with 1, 2, 3, 4, or 5 R⁹ groups; alkenyl optionally substituted with one or two groups selected from carboxy and alkoxycarbonyl; or cycloalkyl optionally substituted with 1 or 2 groups independently selected from hydroxyalkyl, alkoxycarbonyl, carboxy, and —C(O)NR¹⁰R¹⁰; and all other groups are as defined in the Summary of the Invention or as defined in any of embodiments 1, A, B, C, D, E, F, G, Gl, J, J1, J2, J3, and J4. In another embodiment, the Compound of Formula I is that where R⁶ is —OR¹³; —NR¹¹R^{11a}; alkyl substituted with 1, 2, 3, 4, or 5 R⁹ groups; alkenyl optionally substituted with one or two groups independently selected from carboxy and alkoxycarbonyl; or cycloalkyl optionally substituted with 1 or 2 groups independently selected from hydroxyalkyl, alkoxycarbonyl, carboxy, and —C(O)NR¹⁰R^{10a}; and all other groups are as defined in the Summary of the Invention or as defined in any of embodiments 1, A, B, C, D, E, F, G, G1, J, J1, J2, J3, and J4.

Another embodiment (K1) of the Invention is directed to a Compound of Formula I where R⁶ is alkyl substituted with 1, 2, 3, 4, or 5 R⁹ groups; and all other groups are as defined in the Summary of the Invention or as defined in any of embodiments 1, A, B, C, D, E, F, G, G1, J, J1, J2, J3, and J4. Another embodiment of the Invention is directed to a Compound of Formula I where R⁶ is alkyl substituted with 1, 2, or 3 R⁹ groups; and all other groups are as defined in the Summary of the Invention or as defined in any of embodiments 1, A, B, C, D, E, F, G, G1, J, J1, J2, J3, and J4.

Another embodiment (Kla) of the Invention is directed to a Compound of Formula I where R⁶ is alkyl substituted by one R⁹ where R⁹ is —C(O)OR¹⁰ and additionally optionally substituted by one or two R⁹ groups independently selected from cyano, hydroxy, —NR¹¹R^{11a} and —C(O)NR¹⁰R^{10a};and all other groups are as defined in the Summary of the Invention or as defined in any of embodiments 1, A, B, C, D, E, F, G, G1, J, J1, J2, J3, and J4. Another embodiment of the Invention is directed to a Compound of Formula I where R⁶ is alkyl substituted by one R⁹ where R⁹ is —C(O)OR¹⁰ and additionally optionally substituted by one or two R⁹ groups independently selected from cyano, hydroxy, —NR¹¹R^{11a} and —C(O)NR¹⁰R^{10a} where R¹¹ is hydrogen, R^{11a} is hydrogen or alkyl, each R¹⁰ is hydrogen, and R^{10a} is hydrogen; and all other groups are as defined in the Summary of the Invention or as defined in any of embodiments 1, A, B, C, D, E, F, G, G1, J, J1, J2, J3, and J4. Another embodiment of the Invention is directed to a Compound of Formula I where R⁶ is alkyl substituted by one R⁹ where R⁹ is —C(O)OR¹⁰ and R¹⁰ is hydrogen or alkyl; and all other groups are as defined in the Summary of the Invention or as defined in any of embodiments 1, A, B, C, D, E, F, G, G1, J, J1, J2, J3, and J4. Another embodiment of the Invention is directed to a Compound of Formula I where R⁶ is alkyl substituted by one R⁹ where R⁹ is —C(O)OR¹⁰ where R¹⁰ is hydrogen; and all other groups are as defined in the Summary of the Invention or as defined in any of embodiments 1, A, B, C, D, E, F, G, G1, J, J1, J2, J3, and J4. Another embodiment of the Invention is directed to a Compound of Formula I where R⁶ is 2-carboxy-ethyl, 1-carboxy-propyl, or 2-carboxy-propyl; and all other groups are as defined in the Summary of the Invention or as defined in any of embodiments 1, A, B, C, D, E, F, G, G1, J, J1, J2, J3, and J4.

Another embodiment (Klb) of the Invention is directed to a Compound of Formula I where R⁶ is alkyl substituted by one R⁹ where R⁹ is heterocycloalkyl optionally substituted with 1 or 2 groups independently selected from hydroxy, alkoxycarbonyl, carboxy, alkyl, alkoxycarbonylamino, and hydroxyalkyl; and all other groups are as defined in the Summary of the Invention or as defined in any of embodiments 1, A, B, C, D, E, F, G, G1, J, J1, J2, J3, and J4. Another embodiment of the Invention is directed to a Compound of Formula I where R⁶ is alkyl substituted by one R⁹ where R⁹ is heterocycloalkyl optionally substituted with 1 or 2 groups independently selected from hydroxy, carboxy, and hydroxyalkyl; and all other groups are as defined in the Summary of the Invention or as defined in any of embodiments 1, A, B, C, D, E, F, G, G1, J, J1, J2, J3, and J4. Another embodiment of the Invention is directed to a Compound of Formula I where R⁶ is alkyl substituted by one R⁹ where R⁹ is azetidinyl optionally substituted with one carboxy; and all other groups are as defined in the Summary of the Invention or as defined in any of embodiments 1, A, B, C, D, E, F, G, G1, J, J1, J2, J3, and J4. Another embodiment of the Invention is directed to a Compound of Formula I where R⁶ is alkyl substituted by one R⁹ where R⁹ is 5-(tert-butoxycarbonylamino)- 2,2-dimethyl-1,3-dioxan-5-yl; and all other groups are as defined in the Summary of the Invention or as defined in any of embodiments 1, A, B, C, D, E, F, G, G1, J, J1, J2, J3, and J4.

Another embodiment (Klc) of the Invention is directed to a Compound of Formula I where R⁶ is alkyl substituted by one R⁹ where R⁹ is —C(O)NR¹⁰R^{10a}; and all other groups are as defined in the Summary of the Invention or as defined in any of embodiments 1, A, B, C, D, E, F, G, G1, J, J1, J2, J3, and J4. Another embodiment of the Invention is directed to a Compound of Formula I where R⁶ is alkyl substituted by one R⁹ where R⁹ is —C(O)NR¹⁰R^{10a} and R¹⁰ and R^{10a} are hydrogen; and all other groups are as defined in the Summary of the Invention or as defined in any of embodiments 1, A, B, C, D, E, F, G, G1, J, J1, J2, J3, and J4.

Another embodiment (K1d) of the Invention is directed to a Compound of Formula I where R⁶ is alkyl substituted by 1, 2, or 3 R⁹ where each R⁹ is independently hydroxy, carboxy, —NR¹¹R^{11a}, —P(O)(OR¹⁶)₂, or —OP(O)(OR¹⁶)₂; and all other groups are as defined in the Summary of the Invention or as defined in any of embodiments 1, A, B, C, D, E, F, G, G1, J, J1, J2, J3, and J4. Another embodiment of the Invention is directed to a Compound of Formula I where R⁶ is alkyl substituted by 1, 2, or 3 R⁹ where each R⁹ is independently hydroxy, —P(O)(OH)₂, —OP(O)(OH)₂, —NR¹¹R^{11a} and R¹¹ is hydrogen and R^{11a} is carboxyalkyl or hydroxyalkyl; and all other groups are as defined in the Summary of the Invention or as defined in any of embodiments 1, A, B, C, D, E, F, G, G1, J, J1, J2, J3, and J4. Another embodiment of the Invention is directed to a Compound of Formula I where R⁶ is 1-amino-2-carboxy-ethyl, 2-amino-2-carboxy-ethyl, 2-carboxy-1-ethylamino-ethyl, N-(2-carboxyethyl)-amino-methyl, 3-amino-4-hydroxy-3-hydroxymethyl-butyl; and all other groups are as defined in the Summary of the Invention or as defined in any of embodiments 1, A, B, C, D, E, F, G, G1, J, J1, J2, J3, and J4.

Another embodiment (K1e) of the Invention is directed to a Compound of Formula I where R⁶ is alkyl substituted by one or two R⁹ where R⁹ is hydroxy; and all other groups are as defined in the Summary of the Invention or as defined in any of embodiments 1, A, B, C, D, E, F, G, G1, J, J1, J2, J3, and J4.

Another embodiment (K1f) of the Invention is directed to a Compound of Formula I where R⁶ is alkyl substituted by one or two R⁹ where R⁹ is —NR¹²S(O)₂R^{12a}; and all other groups are as defined in the Summary of the Invention or as defined in any of embodiments 1, A, B, C, D, E, F, G, G1, J, J1, J2, J3, and J4. Another embodiment of the Invention is directed to a Compound of Formula I where R⁶ is alkyl substituted by one or two R⁹ where R⁹ is —NR¹²S(O)₂R^{12a} and R¹² is hydrogen or alkyl and R^{12a} is alkyl; and all other groups are as defined in the Summary of the Invention or as defined in any of embodiments 1, A, B, C, D, E, F, G, G1, J, J1, J2, J3, and J4.

Another embodiment (K1g) of the Invention is directed to a Compound of Formula I where R⁶ is alkyl substituted by one or two R⁹ where each R⁹ is —OP(O)(OR¹⁶)₂; and all other groups are as defined in the Summary of the Invention or as defined in any of embodiments 1, A, B, C, D, E, F, G, G1, J, J1, J2, J3, and J4. Another embodiment of the Invention is directed to a Compound of Formula where R⁶ is alkyl substituted by one or two R⁹ where each R⁹ is —OP(O)(OR¹⁶)₂ and each R¹⁶ is hydrogen; and all other groups are as defined in the Summary of the Invention or as defined in any of embodiments 1, A, B, C, D, E, F, G, G1, J, J1, J2, J3, and J4.

Another embodiment (K1h) of the Invention is directed to a Compound of Formula I where R⁶ is alkyl substituted by one or two R⁹ where each R⁹ is —OS(O)₂OH; and all other groups are as defined in the Summary of the Invention or as defined in any of embodiments 1, A, B, C, D, E, F, G, G1, J, J1, J2, J3, and J4.

Another embodiment (KW of the Invention is directed to a Compound of Formula I where R⁶ is alkyl substituted by one or two R⁹ where each R⁹ is —P(O)(OR¹⁶)₂; and all other groups are as defined in the Summary of the Invention or as defined in any of embodiments 1, A, B, C, D, E, F, G, G1, J, J1, J2, J3, and J4. Another embodiment of the Invention is directed to a Compound of Formula where R⁶ is alkyl substituted by one or two R⁹ where each R⁹ is —P(O)(OR¹⁶)₂ and each R¹⁶ is hydrogen; and all other groups are as defined in the Summary of the Invention or as defined in any of embodiments 1, A, B, C, D, E, F, G, G1, J, J1, J2, J3, and J4.

Another embodiment (K1k) of the Invention is directed to a Compound of Formula I where R⁶ is alkyl substituted by one R⁹ where the R⁹ is —S(O)ₙR¹⁸; and all other groups are as defined in the Summary of the Invention or as defined in any of embodiments 1, A, B, C, D, E, F, G, G1, J, J1, J2, J3, and J4. In another embodiment, the Compound of Formula I is that where R⁶ is alkyl substituted by one R⁹ where the R⁹ is —S(O)ₙR¹⁸ and n is 2; and all other groups are as defined in the Summary of the Invention or as defined in any of embodiments 1, A, B, C, D, E, F, G, G1, J, J1, J2, J3, and J4. In another embodiment, the Compound of Formula I is that where R⁶ is —CH₂S(O)₂CH₃; and all other groups are as defined in the Summary of the Invention or as defined in any of embodiments 1, A, B, C, D, E, F, G, G1, J, J1, J2, J3, and J4.

Another embodiment (K1m) of the Invention is directed to a Compound of Formula I where R⁶ is alkyl substituted by 1, 2, or 3 R⁹ where each R⁹ is independently hydroxy, carboxy, amino, or —OP(O)(OR¹⁶)₂; and all other groups are as defined in the Summary of the Invention or as defined in any of embodiments 1, A, B, C, D, E, F, G, G1, J, J1, J2, J3, and J4.

Another embodiment (K2) of the Invention is directed to a Compound of Formula I where R⁶ is alkenyl optionally substituted with one or two groups independently selected from alkoxycarbonyl and carboxy; and all other groups are as defined in the Summary of the Invention or as defined in any of embodiments 1, A, B, C, D, E, F, G, G1, J, J1, J2, J3, and J4. Another embodiment of the Invention is directed to a Compound of Formula I where R⁶ is alkenyl optionally substituted with one or two carboxy; and all other groups are as defined in the Summary of the Invention or as defined in any of embodiments 1, A, B, C, D, E, F, G, G1, J, J1, J2, J3, and J4. Another embodiment of the Invention is directed to a Compound of Formula I where R⁶ is alkenyl optionally substituted with one carboxy; and all other groups are as defined in the Summary of the Invention or as defined in any of embodiments 1, A, B, C, D, E, F, G, G1, J, J1, J2, J3, and J4.

Another embodiment (K3) of the Invention is directed to a Compound of Formula I where R⁶ is cycloalkyl optionally substituted with 1 or 2 groups independently selected from hydroxyalkyl, alkoxycarbonyl, carboxy, and —C(O)NR¹⁰R^{10a}; and all other groups are as defined in the Summary of the Invention or as defined in any of embodiments 1, A, B, C, D, E, F, G, G1, J, J1, J2, J3, and J4. In another embodiment the Compound of Formula I is that where R⁶ is cycloalkyl optionally substituted with 1 or 2 groups independently selected from hydroxyalkyl, carboxy, and —C(O)NR¹⁰R^{10a}; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is cyclopropyl optionally substituted with 1 or 2 groups independently selected from hydroxyalkyl, carboxy, and —C(O)NR¹⁰R^{10a}; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is cyclopropyl optionally substituted with carboxy, C(O)NH₂, or hydroxymethyl; and all other groups are as defined in the Summary of the Invention or as defined in any of embodiments 1, A, B, C, D, E, F, G, G1, J, J1, J2, J3, and J4.

Another embodiment (K4) of the Invention is directed to a Compound of Formula I where R⁶ is —NR¹¹R^{11a}; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —NR¹¹R^{11a} and R¹¹ is hydrogen and R^{11a} is hydrogen, hydroxyalkyl, or carboxyalkyl; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —NR¹¹R^{11a} and R¹¹ is hydrogen and R^{11a} is hydroxyalkyl or carboxyalkyl; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —NR¹¹R^{11a} and R^{H} is hydrogen and R^{11a} is 2-hydroxyethyl, 2,3-dihydroxyprop-1-yl, or 2-carboxyethyl; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —NR¹¹R^{11a} and R¹¹ and R^{11a} are hydrogen; and all other groups are as defined in the Summary of the Invention or as defined in any of embodiments 1, A, B, C, D, E, F, G, G1, J, J1, J2, J3, and J4.

Another embodiment (K5) of the Invention is directed to a Compound of Formula I where R⁶ is —NR¹²S(O)₂R^{12a}; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —NHS(O)₂R^{12a}; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —NHS(O)₂R^{12a} and R^{12a} is alkyl, alkenyl, alkylaminoalkyl, or dialkylaminoalkyl; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —NHS(O)₂R^{12a} and R^{12a} is methyl, vinyl, or 2-(N,N-diethylamino)-ethyl; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —NHS(O)₂R^{12a} and R^{12a} is alkyl; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —NHS(O)₂R^{12a} and R^{12a} is methyl; and all other groups are as defined in the Summary of the Invention or as defined in any of embodiments 1, A, B, C, D, E, F, G, G1, J, J1, J2, J3, and J4.

Another embodiment (K6) of the Invention is directed to a Compound of Formula I where R⁶ is optionally substituted heteroaryl or R⁶ is optionally substituted heterocycloalkyl; and all other groups are as defined in the Summary of the Invention or as defined in any of embodiments 1, A, B, C, D, E, F, G, G1, J, J1, J2, J3, and J4.

Another embodiment (K7) of the Invention is directed to a Compound of Formula I where R⁶ is halo; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is chloro, fluoro, or bromo; and all other groups are as defined in the Summary of the Invention or as defined in any of embodiments 1, A, B, C, D, E, F, G, G1, J, J1, J2, J3, and J4.

Another embodiment (K8) of the Invention is directed to a Compound of Formula I where R⁶ is hydroxy or —OR¹³; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is hydroxy; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —OR¹³; and all other groups are as defined in the Summary of the Invention or as defined in any of embodiments 1, A, B, C, D, E, F, G, G1, J, J1, J2, J3, and J4.

In another embodiment (K9), the Invention is directed to a Compound of Formula I where R⁶ is —OR¹³ and R¹³ is alkenyl; and all other groups are as defined in the Summary of the Invention or as defined in any of embodiments 1, A, B, C, D, E, F, G, G1, J, J1, J2, J3, and J4.

In another embodiment (K10), the Invention is directed to a Compound of Formula I where R⁶ is —OR¹³ and R¹³ is alkyl substituted with 1, 2, 3, or 4 groups independently selected from halo, hydroxy, alkoxy, alkylsulfanyl, alkylsulfonyl, cyano, —C(O)OR¹⁰, —OC(O)R^{10b}, —C(O)R^{10b}, —NR¹¹R^{11a}, —P(O)(OR¹⁶)₂, —OP(O)(OR¹⁶)₂, —OS(O)₂OH, —OSi(alkyl)₃, and heterocycloalkyl where the heterocycloalkyl is optionally substituted with one, two, or three groups independently selected from alkyl, carboxy, alkoxycarbonyl, alkoxycarbonylamino, and phenyl; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —OR¹³ and R¹³ is alkyl substituted with 1, 2, 3, or 4 groups independently selected from halo, hydroxy, alkoxy, —C(O)OR¹⁰, —OC(O)R^{10b}, —C(O)R^{10b}, —NR¹¹R^{11a}, —P(O)(OR¹⁶)₂, —OP(O)(OR¹⁶)₂, —OS(O)₂OH, and heterocycloalkyl where the heterocycloalkyl is optionally substituted with one or two alkyl; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —OR¹³ and R¹³ is alkyl substituted with 1, 2, 3, or 4 groups independently selected from halo, hydroxy, —C(O)R^{10b}, —NR¹¹R^{11a}, —P(O)(OR¹⁶)₂, and —OP(O)(OR¹⁶)₂; and all other groups are defined in the Summary of the Invention or as defined in any of embodiments 1, A, B, C, D, E, F, G, G1, J, J1, J2, J3, and J4.

In another embodiment (K10a), the Invention is directed to a Compound of Formula I where R⁶ is —OR¹³ and R¹³ is alkyl substituted with one —NR¹¹R^{11a} and one —C(O)OR¹⁰; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —OR¹³ and R¹³ is alkyl substituted with one —NR¹¹R^{11a} and one —C(O)OR¹⁰ where R¹¹ is hydrogen, R^{11a} is hydrogen, and R¹⁰ is hydrogen; and all other groups are as defined in the Summary of the Invention or as defined in any of embodiments 1, A, B, C, D, E, F, G, G1, J, J1, J2, J3, and J4.

In another embodiment (K10b), the Invention is directed to a Compound of Formula I where R⁶ is —OR¹³ and R¹³ is alkyl substituted with one —NR¹¹R^{11a} and one or two hydroxy; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —OR¹³ and R¹³ is alkyl substituted with one —NR¹¹R^{11a} and one or two hydroxy where R¹¹ is hydrogen and R^{11a} is hydrogen; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is 2-amino-3-hydroxypropyloxy, 2R-amino-3-hydroxypropyloxy, or 2S-amino-3-hydroxypropyloxy; and all other groups are as defined in the Summary of the Invention or as defined in any of embodiments 1, A, B, C, D, E, F, G, G1, J, J1, J2, J3, and J4.

In another embodiment (K10c), the Invention is directed to a Compound of Formula I where R⁶ is —OR¹³ and R¹³ is alkyl substituted with one or two hydroxy; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is 3-hydroxy-2,2-dimethyl-propyloxy, 2-hydroxy-2,2-dimethyl-ethyloxy, 2-hydroxy-ethyloxy, (1,3-dihydroxypropan-2-yl)oxy, 2-hydroxy-l-methyl-ethyloxy, 2-hydroxy-1R-methyl-ethyloxy, 2-hydroxy-1S-methyl-ethyloxy, (2,3-dihydroxypropyl)oxy, (2R)-2,3-dihydroxypropyloxy, (2S)-2,3-dihydroxypropyloxy, (2-hydroxypropyl)oxy, (2R-hydroxypropyl)oxy, or (2S-hydroxypropyl)oxy; and all other groups are as defined in the Summary of the Invention or as defined in any of embodiments 1, A, B, C, D, E, F, G, G1, J, J1, J2, J3, and J4.

In another embodiment (K1 0d), the Invention is directed to a Compound of Formula I where R⁶ is —OR¹³ and R¹³ is alkyl substituted with one —NR¹¹R^{11a}and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —OR¹³ and R¹³ is alkyl substituted with one —NR¹¹R^{11a} and R¹¹ and R^{11a} are independently hydrogen or hydroxyalkyl; and all other groups are as defined in the Summary of the Invention or as defined in any of embodiments 1, A, B, C, D, E, F, G, G1, J, J1, J2, J3, and J4.

In another embodiment (K10e), the Invention is directed to a Compound of Formula I where R⁶ is —OR¹³ and R¹³ is alkyl substituted with one —C(O)OR¹⁶; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —OR¹³ and R¹³ is alkyl substituted with one —C(O)OR¹⁰ and R¹⁰ is hydrogen or alkyl; and all other groups are as defined in the Summary of the Invention or as defined in any of embodiments 1, A, B, C, D, E, F, G, G1, J, J1, J2, J3, and J4.

In another embodiment (K10f), the Invention is directed to a Compound of Formula I where R⁶ is —OR¹³ and R¹³ is alkyl substituted with one, two, three, or four groups selected from hydroxy and halo; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —OR¹³ and R¹³ is alkyl substituted with one hydroxy and one, two, or three halo; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —OR¹³ and R¹³ is alkyl substituted with one hydroxy and one, two, or three fluoro; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is 2,2-difluoro-3-hydroxy-propyloxy, 3-fluoro-2-hydroxy-propyloxy, 2R-3-fluoro-2-hydroxy-propyloxy, or 2S-3-fluoro-2-hydroxy-propyloxy; and all other groups are as defined in the Summary of the Invention or as defined in any of embodiments 1, A, B, C, D, E, F, G, G1, J, J1, J2, J3, and J4.

In another embodiment (K10g), the Invention is directed to a Compound of Formula I where R⁶ is —OR¹³ and R¹³ is alkyl substituted with one or two alkoxy; and all other groups are as defined in the Summary of the Invention or as defined in any of embodiments 1, A, B, C, D, E, F, G, G1, J, J1, J2, J3, and J4. In another embodiment (K10g), the Invention is directed to a Compound of Formula I where R⁶ is —OR¹³ and R¹³ is alkyl substituted with one or two methoxy; and all other groups are as defined in the Summary of the Invention or as defined in any of embodiments 1, A, B, C, D, E, F, G, G1, J, J1, J2, J3, and J4.

In another embodiment (K10j), the Invention is directed to a Compound of Formula I where R⁶ is —OR¹³ and R¹³ is alkyl substituted with one —OC(O)R^{10b}; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —OR¹³ and R¹³ is alkyl substituted with one —OC(O)R^{10b} and R^{10b} is alkyl; and all other groups are as defined in the Summary of the Invention or as defined in any of embodiments 1, A, B, C, D, E, F, G, G1, J, J1, J2, J3, and J4.

In another embodiment (K10k), the Invention is directed to a Compound of Formula I where R⁶ is —OR¹³ and R¹³ is alkyl substituted with one hydroxy and one —C(O)OR¹⁰; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —OR¹³ and R¹³ is alkyl substituted with one hydroxy and one —C(O)OR¹⁰ and R¹⁰ is hydrogen or alkyl; and all other groups are as defined in the Summary of the Invention or as defined in any of embodiments 1, A, B, C, D, E, F, G, G1, J, J1, J2, J3, and J4.

In another embodiment (K10m), the Invention is directed to a Compound of Formula I where R⁶ is —OR¹³ and R¹³ is alkyl substituted with one —C(O)R^{10b}; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —OR¹³ and R¹³ is alkyl substituted with one —C(O)R^{10b} and R^{10b} is alkyl, haloalkyl, hydroxyalkyl, carboxyalkyl, or alkyl substituted with one or two groups independently selected from —P(O)(OR¹⁶)₂, —OP(O)(OR¹⁶)₂, —OS(O)₂OH, and —OSi(alkyl)₃; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —OR¹³ and R¹³ is alkyl substituted with one —C(O)R^{10b} and R^{10b} is alkyl, haloalkyl, hydroxyalkyl, or alkyl substituted with one —P(O)(OR¹⁶)₂, —OP(O)(OR¹⁶)₂, or —OS(O)₂OH; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is 2-oxo-propyloxy, 3-hydroxy-2-oxo-propyloxy, or [2-oxo-3-(phosphonooxy)propyl]oxy; and all other groups are as defined in the Summary of the Invention or as defined in any of embodiments 1, A, B, C, D, E, F, G, G1, J, J1, J2, J3, and J4.

In another embodiment (K10n), the Invention is directed to a Compound of Formula I where R⁶ is —OR¹³ and R¹³ is alkyl substituted with heterocycloalkyl where the heterocycloalkyl is optionally substituted with one or two alkyl; and all other groups are as defined in the Summary of the Invention or as defined in any of embodiments 1, A, B, C, D, E, F, G, G1, J, J1, J2, J3, and J4.

In another embodiment (K10p), the Invention is directed to a Compound of Formula I where R⁶ is —OR¹³ and R¹³ is alkyl substituted with one hydroxy and one alkoxy; and all other groups are as defined in the Summary of the Invention or as defined in any of embodiments 1, A, B, C, D, E, F, G, G1, J, J1, J2, J3, and J4.

In another embodiment (K10q), the Invention is directed to a Compound of Formula I where R⁶ is —OR¹³ and R¹³ is alkyl substituted with 1, 2, or 3 groups selected from amino, hydroxy, halo, and —OP(O)(OR¹⁶)₂; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —OR¹³ and R¹³ is alkyl substituted with one or two —OP(O)(OH)₂ and optionally additionally with one fluoro; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —OR¹³ and R¹³ is alkyl substituted with one or two —OP(O)(OH)₂ and optionally additionally with one amino; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —OR¹³ and R¹³ is alkyl substituted with one —OP(O)(OH)₂ and optionally additionally with one hydroxy; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is [2-amino-3-(phosphonooxy)propyl]oxy, [(2R)-2-amino-3-(phosphonooxy)propyl]oxy, [(25)-2-amino-3-(phosphonooxy)propyl]oxy, [1-(phosphonooxy)propan-2-yl]oxy, [(2S)-1-(phosphonooxy)propan-2-yl]oxy, [(2R)-1-(phosphonooxy)propan-2-yl]oxy, [2-hydroxy-3-(phosphonooxy)propyl]oxy, [(2R)-2-hydroxy-3-(phosphonooxy)propyl]oxy, [(2S)-2-hydroxy-3-(phosphonooxy)propyl]oxy, [2-(phosphonooxy)propyl]oxy, [(2R)-2-(phosphonooxy)propyl]oxy, [(2S)-2-(phosphonooxy)propyl]oxy, [3-fluoro-2-(phosphonooxy)propyl]oxy, [(2R)-3-fluoro-2-(phosphonooxy)propyl]oxy, or [(2S)-3-fluoro-2-(phosphonooxy)propyl]oxy; and all other groups are as defined in the Summary of the Invention or as defined in any of embodiments 1, A, B, C, D, E, F, G, G1, J, J1, J2, J3, and J4.

In another embodiment (K10r), the Invention is directed to a Compound of Formula I where R⁶ is —OR¹³ and R¹³ is alkyl substituted with one —P(O)(OR¹⁶)₂ and optionally additionally with one hydroxy; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —OR¹³ and R¹³ is alkyl substituted with one —P(O)(OH)₂ and optionally additionally with one hydroxy; and all other groups are as defined in the Summary of the Invention or as defined in any of embodiments 1, A, B, C, D, E, F, G, G1, J, J1, J2, J3, and J4.

In another embodiment (K10s), the Invention is directed to a Compound of Formula I where R⁶ is —OR¹³ and R¹³ is alkyl substituted with one —OS(O)₂OH; and all other groups are as defined in the Summary of the Invention or as defined in any of embodiments 1, A, B, C, D, E, F, G, G1, J, J1, J2, J3, and J4.

In another embodiment (K10t), the Invention is directed to a Compound of Formula I where R⁶ is —OR¹³ and R¹³ is alkyl substituted with one alkylsulfonyl; and all other groups are as defined in the Summary of the Invention or as defined in any of embodiments 1, A, B, C, D, E, F, G, G1, J, J1, J2, J3, and J4.

In another embodiment (K11), the Invention is directed to a Compound of Formula I where R⁶ is —OR¹³ and R¹³ is heterocycloalkyl optionally substituted with 1 or 2 groups independently selected from alkyl, carboxy, hydroxyalkyl, carboxyalkyl, and phenyl; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —OR¹³ and R¹³ is heterocycloalkyl optionally substituted with 1 or 2 groups independently selected from alkyl, carboxy, hydroxyalkyl, and carboxyalkyl; and all other groups are as defined in the Summary of the Invention or as defined in any of embodiments 1, A, B, C, D, E, F, G, G1, J, J1, J2, J3, and J4. In another embodiment, the Invention is directed to a Compound of Formula I where R⁶ is —OR¹³ and R¹³ is pyrrolidinyl optionally substituted with 1 or 2 groups independently selected from alkyl, carboxy, hydroxyalkyl, and carboxyalkyl; and all other groups are as defined in the Summary of the Invention or as defined in any of embodiments 1, A, B, C, D, E, F, G, G1, J, J1, J2, J3, and J4.

In another embodiment (K12), the Invention is directed to a Compound of Formula I where R⁶ is —C(O)H, cyano, carboxy, alkoxycarbonyl, —C(═NOH)NH₂, or —C(O)R¹⁷; and all other groups are as defined in the Summary of the Invention or as defined in any of embodiments 1, A, B, C, D, E, F, G, G1, J, J1, J2, J3, and J4.

In another embodiment (K13), the Invention is directed to a Compound of Formula I where R⁶ is —OR¹³ or R⁶ is alkyl substituted with 1, 2, or 3 R⁹; and all other groups are as defi in the Summary of the Invention or as defined in embodiments 1, A, B, C, D, E, F, G, G1, J, J1, J2, J3, and J4. In another embodiment the Compound of Formula I is that where R⁶ is 2-carboxy-ethyl, 3-amino-4-hydroxy-3-hydroxymethyl-butyl, 2-amino-3-hydroxypropyloxy, 2R-amino-3-hydroxypropyloxy, or 2S-amino-3-hydroxypropyloxy, [2-amino-3-(phosphonooxy)propyl]oxy, [(2R)-2-amino-3-(phosphonooxy)propyl]oxy, [(2S)-2-amino-3-(phosphonooxy)propyl]oxy, 3-hydroxy-2,2-dimethyl-propyloxy, 2-hydroxy-2,2-dimethyl-ethyloxy, 2-hydroxy-ethyloxy, (1,3-dihydroxypropan-2-yl)oxy, 2-hydroxy-1-methyl-ethyloxy, 2-hydroxy-1R-methyl-ethyloxy, 2-hydroxy-1S-methyl-ethyloxy, (2,3-dihydroxypropyl)oxy, (2R)-2,3-dihydroxypropyloxy, (2S)-2,3-dihydroxypropyloxy, (2-hydroxypropyl)oxy, (2R-hydroxypropyl)oxy, or (2S-hydroxypropyl)oxy, [1-(phosphonooxy)propan-2-yl]oxy, [(2S)-1-(phosphonooxy)propan-2-yl]oxy, [(2R)-1-(phosphonooxy)propan-2-yl]oxy, [2-hydroxy-3-(phosphonooxy)propyl]oxy, [(2R)-2-hydroxy-3-(phosphonooxy)propyl]oxy, [(2S)-2-hydroxy-3-(phosphonooxy)propyl]oxy, [2-(phosphonooxy)propyl]oxy, [(2R)-2-(phosphonooxy)propyl]oxy, [(2S)-2-(phosphonooxy)propyl]oxy, 2,2-difluoro-3-hydroxy-propyloxy, 3-fluoro-2-hydroxy-propyloxy, 2R-3-fluoro-2-hydroxy-propyloxy, or 2S-3-fluoro-2-hydroxy-propyloxy, [3-fluoro-2-(phosphonooxy)propyl]oxy, [(2R)-3-fluoro-2-(phosphonooxy)propyl]oxy, [(2S)-3-fluoro-2-(phosphonooxy)propyl]oxy, 2-oxo-propyloxy or 3-hydroxy-2-oxo-propyloxy, or [2-oxo-3-(phosphonooxy)propyl]oxy; and all other groups are as defined in the Summary of the Invention or as defined in any of embodiments 1, A, B, C, D, E, F, G, G1, J, J1, J2, J3, and J4.

Another embodiment (L) of the Invention is directed to a Compound of Formula I where R⁷ and R⁸ are independently hydrogen, halo, haloalkyl, or alkyl; and all other groups are as defined in the Summary of the Invention or as defined in any of embodiments 1, A, B, C, D, E, F, G, G1, J, J1, J2, J3, J4, K, K1, K1a-K1m, K2-K10, K10a-K10t, and K11-K13. Another embodiment of the Invention is directed to a Compound of Formula I where R⁷ is hydrogen, alkyl, or halo and R⁸ is hydrogen, halo, alkyl, or haloalkyl; and all other groups are as defined in the Summary of the Invention or as defined in any of embodiments 1, A, B, C, D, E, F, G, G1, J, J1, J2, J3, J4, K, K1, K1a-K1m, K2-K10, K10a-K10t, and K11-K13. In another embodiment, the Invention is directed to a Compound of Formula I wherein R⁷ is hydrogen, methyl, bromo, chloro, or fluoro and R⁸ is hydrogen, bromo, chloro, fluoro, methyl, or trifluoromethyl; and all other groups are as defined in the Summary of the Invention or as defined in any of embodiments 1, A, B, C, D, E, F, G, G1, J, J1, J2, J3, J4, K, K1, K1a-K1m, K2-K10, K10a-K10t, and K11-K13.

Another embodiment (L1) of the Invention is directed to a Compound of Formula I wherein R⁷ is hydrogen and R⁸ is halo; and all other groups are as defined in the Summary of the Invention or as defined in any of embodiments 1, A, B, C, D, E, F, G, G1, J, J1, J4, K, K1, K1a-K1m, K2-K10, K10a-K10t, and K11-K13. In another embodiment, the Invention is directed to a Compound of Formula I wherein R⁷ is hydrogen and R⁸ is chloro; and all other groups are as defined in the Summary of the Invention or as defined in any of embodiments 1, A, B, C, D, E, F, G, G1, J, J1, J4, K, K1, K1a-K1m, K2-K10, K10a-K10t, and K11-K13. In another embodiment, the Invention is directed to a Compound of Formula I wherein R⁷ is hydrogen and R⁸ is fluoro; and all other groups are as defined in the Summary of the Invention or as defined in any of embodiments 1, A, B, C, D, E, F, G, G1, J, J1, J4, K, K1, K1a-K1m, K2-K10, K10a-K10t, and K11-K13. In another embodiment, the Invention is directed to a Compound of Formula I wherein R⁷ is hydrogen and R⁸ is bromo; and all other groups are as defined in the Summary of the Invention or as defined in any of embodiments 1, A, B, C, D, E, F, G, G1, J, J1, J4, K, K1, K1a-K1m, K2-K10, K10a-K10t, and K11-K13.

Another embodiment (L2) of the Invention is directed to a Compound of Formula I wherein R⁷ is halo and R⁸ is halo; and all other groups are as defined in the Summary of the Invention or as defined in any of embodiments 1, A, B, C, D, E, F, G, G1, J, J1, J2, J3, J4, K, K1, K1a-K1m, K2-K10, K10a-K10t, and K11-K13. In another embodiment, the Invention is directed to a Compound of Formula I wherein R⁷ is chloro or fluoro and R⁸ is chloro or fluoro; and all other groups are as defined in the Summary of the Invention or as defined in any of embodiments 1, A, B, C, D, E, F, G, G1, J, J1, J2, J3, J4, K, K1, K1a-K1m, K2-K10, K10a-K10t, and K11-K13. In another embodiment, the Invention is directed to a Compound of Formula I wherein R⁷ and R⁸ are chloro; and all other groups are as defined in the Summary of the Invention or as defined in any of embodiments 1, A, B, C, D, E, F, G, G1, J, J1, J2, J3, J4, K, K1, K1a-K1m, K2-K10, K10a-K10t, and K11-K13. In another embodiment, the Invention is directed to a Compound of Formula I wherein R⁷ and R⁸ are fluoro; and all other groups are as defined in the Summary of the Invention or as defined in any of embodiments 1, A, B, C, D, E, F, G, G1, J, J1, J2, J3, J4, K, K1, K1a-K1m, K2-K10, K10a-K10t, and K11-K13. In another embodiment, the Invention is directed to a Compound of Formula I wherein R⁷ is chloro and R⁸ is fluoro; and all other groups are as defined in the Summary of the Invention or as defined in any of embodiments 1, A, B, C, D, E, F, G, G1, J, J1, J2, J3, J4, K, K1, K1a-K1m, K2-K10, K10a-K10t, and K11-K13. In another embodiment, the Invention is directed to a Compound of Formula I wherein R⁷ is fluoro and R⁸ is chloro; and all other groups are as defined in the Summary of the Invention or as defined in any of embodiments 1, A, B, C, D, E, F, G, G1, J, J1, J2, J3, J4, K, K1, K1a-K1m, K2-K10, K10a-K10t, and K11-K13.

Another embodiment (L3) of the Invention is directed to a Compound of Formula I wherein R⁷ is hydrogen and R⁸ is haloalkyl; and all other groups are as defined in the Summary of the Invention or as defined in any of embodiments 1, A, B, C, D, E, F, G, G1, J, J1, J4, K, K1, K1a-K1m, K2-K10, K10a-K10t, and K11-K13. In another embodiment, the Invention is directed to a Compound of Formula I wherein R⁷ is hydrogen and R⁸ is trifluoromethyl; and all other groups are as defined in the Summary of the Invention or as defined in any of embodiments 1, A, B, C, D, E, F, G, G1, J, J1, J4, K, K1, K1a-K1m, K2-K10, K10a-K10t, and K11-K13.

Another embodiment (L4) of the Invention is directed to a Compound of Formula I wherein R⁷ is hydrogen and R⁸ is alkyl; and all other groups are as defined in the Summary of the Invention or as defined in any of embodiments 1, A, B, C, D, E, F, G, G1, J, J1, J4, K, K1, K1a-K1m, K2-K10, K10a-K10t, and K11-K13. In another embodiment, the Invention is directed to a Compound of Formula I wherein R⁷ is hydrogen and R⁸ is methyl; and all other groups are as defined in the Summary of the Invention or as defined in any of embodiments 1, A, B, C, D, E, F, G, G1, J, J1, J4, K, K1, K1a-K1m, K2-K10, K10a-K10t, and K11-K13.

Another embodiment (L5) of the Invention is directed to a Compound of Formula I wherein R⁷ is halo and R⁸ is alkyl; and all other groups are as defined in the Summary of the Invention or as defined in any of embodiments 1, A, B, C, D, E, F, G, G1, J, J1, J2, J3, J4, K, K1, K1a-K1m, K2-K10, K10a-K10t, and K11-K13. In another embodiment, the Invention is directed to a Compound of Formula I wherein R⁷ is fluoro or chloro and R⁸ is methyl; and all other groups are as defined in the Summary of the Invention or as defined in any of embodiments 1, A, B, C, D, E, F, G, G1 J, J1, J2, J3, J4, K, K1, K1a-K1m, K2-K10, K10a-K10t, and K11-K13. In another embodiment, the Invention is directed to a Compound of Formula I wherein R⁷ is fluoro and R⁸ is methyl; and all other groups are as defined in the Summary of the Invention or as defined in any of embodiments 1, A, B, C, D, E, F, G, G1, J, J1, J2, J3, J4, K, K1, K1a-K1m, K2-K10, K10a-K10t, and K11-K13. In another embodiment, the Invention is directed to a Compound of Formula I wherein R⁷ is chloro and R⁸ is methyl; and all other groups are as defined in the Summary of the Invention or as defined in any of embodiments 1, A, B, C, D, E, F, G, G1, J, J1, J2, J3, J4, K, K1, K1a-K1m, K2-K10, K10a-K10t, and K11-K13.

Another embodiment (L6) of the Invention is directed to a Compound of Formula I wherein R⁷ and R⁸ are independently alkyl; and all other groups are as defined in the Summary of the Invention or as defined in any of embodiments 1, A, B, C, D, E, F, G, G1, J, J1, J2, J3, J4, K, K1, K1a-K1m, K2-K10, K10a-K10t, and K11-K13. In another embodiment, the Invention is directed to a Compound of Formula I wherein R⁷ and R⁸ are methyl; and all other groups are as defined in the Summary of the Invention or as defined in any of embodiments 1, A, B, C, D, E, F, G, G1, J, J1, J2, J3, J4, K, K1, K1a-K1m, K2-K10, K10a-K10t, and K11-K13.

Another embodiment (M1) of the invention is directed to a Compound of the Invention where R⁵ is phenyl substituted with R⁶, R⁷, and R⁸; R⁸ is halo, R⁷ is hydrogen or halo; and R¹, R², R³, R⁴, and R⁶ are as defined in the Summary of the Invention for a Compound of Formula I. Another embodiment of the invention is directed to a Compound of the Invention where R¹ is halo; R² and R⁴ are hydrogen; R³ is haloalkyl; R⁵ is phenyl substituted with R⁶, R⁷, and R⁸; R⁸ is halo; R⁷ is hydrogen or halo; and R⁶ is as defined in the Summary of the Invention for a Compound of Formula I.

Another embodiment (M2) of the invention is directed to a Compound of the Invention where R⁵ is phenyl substituted with R⁶, R⁷, and R⁸; R⁸ is halo; R⁷ is hydrogen, halo, or alkyl; and R⁶ is OR¹³, R⁶ is alkyl substituted with one or two R⁹, R⁶ is —NR¹¹R^{11a}, or R⁶ is —NR¹²S(O)₂R^{12a}; and all other groups are as defined in the Summary of the Invention for a Compound of Formula I. Another embodiment of the invention is directed to a Compound of the Invention where R¹ is halo; R² and R⁴ are hydrogen; R³ is haloalkyl; R⁵ is phenyl substituted with R⁶, R⁷, and R⁸; R⁸ is halo; R⁷ is hydrogen, halo, or alkyl; and R⁶ is OR¹³, R⁶ is alkyl substituted with one or two R⁹, R⁶ is —NR¹¹R^{11a}, or R⁶ is —NR¹²S(O)₂R^{12a}; and all other groups are as defined in the Summary of the Invention for a Compound of Formula I. Another embodiment of the invention is directed to a Compound of the Invention where R¹ is halo; R² and R⁴ are hydrogen; R³ is haloalkyl; R⁵ is phenyl substituted with R⁶, R⁷, and R⁸; R⁸ is halo; R⁷ is hydrogen, halo, or alkyl; and R⁶ is OR¹³ or R⁶ is alkyl substituted with one or two R⁹; and all other groups are as defined in the Summary of the Invention for a Compound of Formula I.

Another embodiment (M3) of the invention is directed to a Compound of the Invention where R⁵ is phenyl substituted with R⁶, R⁷, and R⁸; R⁸ is halo; R⁷ is hydrogen, halo, or alkyl; and R⁶ is OR¹³ where R¹³ is alkyl substituted with 1, 2, 3, or 4 groups independently selected from halo, hydroxy, —C(O)R^{10b}, —C(O)OR¹⁰, —NR¹¹R^{11a}, —P(O)(OR¹⁶)₂, and —OP(O)(OR¹⁶)₂; or R⁶ is alkyl substituted with one or two R⁹ where each R⁹ is independently hydroxy, —P(O)(OR¹⁶)₂, —OP(O)(OR¹⁶)₂, or —C(O)OR¹⁰; or R⁶ is —NHR^{11a} and R^{11a} is hydroxyalkyl; or R⁶ is —NHS(O)₂R^{12a} and R^{12a} is alkyl; and all other groups are as defined in the Summary of the Invention for a Compound of Formula I. Another embodiment of the invention is directed to a Compound of the Invention where R⁵ is phenyl substituted with R⁶, R⁷, and R⁸; R⁸ is halo; R⁷ is hydrogen, halo, or alkyl; and R⁶ is OR¹³ where R¹³ is alkyl substituted with one or two groups independently selected from hydroxy, —C(O)R^{10b}, —NR¹¹R^{11a}, —P(O)(OR¹⁶)₂, and —OP(O)(OR¹⁶)₂, and R¹³ alkyl is additionally optionally substituted with one hydroxy or 1, 2, or 3 halo; or R⁶ is alkyl substituted with one or two R⁹ where each R⁹ is independently hydroxy, —P(O)(OR¹⁶)₂, —OP(O)(OR¹⁶)₂, or —C(O)OR¹⁰; or R⁶ is —NHR^{11a} and R^{11a} is hydroxyalkyl; or R⁶ is —NHS(O)₂R^{12a} and R^{12a} is alkyl; and all other groups are as defined in the Summary of the Invention for a Compound of Formula I.

Another embodiment (M4) of the invention is directed to a Compound of the Invention where R⁵ is phenyl substituted with R⁶, R⁷, and R⁸; R⁸ is halo; R⁷ is hydrogen, halo, or alkyl; and R⁶ is —OR¹³ where R¹³ is alkyl substituted with one or two groups independently selected from hydroxy, —C(O)R^{10b}, —NHR^{11a}, —P(O)(OR¹⁶)₂, and —OP(O)(OR¹⁶)₂, and the R¹³ alkyl is additionally optionally substituted with one hydroxy or 1, 2, or 3 halo; or R⁶ is alkyl substituted with one or two R⁹ where each R⁹ is independently hydroxy, —P(O)(OR¹⁶)₂, —OP(O)(OR¹⁶)₂, or —C(O)OR¹⁰; or R⁶ is —NHR^{11a}; or R⁶ is —NHS(O)₂R^{12a} and R^{12a} is alkyl; R¹⁰ is hydrogen; R^{10b} is alkyl, haloalkyl, hydroxyalkyl, carboxyalkyl, or alkyl substituted with one —OP(O)(OR¹⁶)₂; R^{11a} is hydrogen, alkyl, or hydroxyalkyl; R^{12a} is alkyl; R¹⁶ is hydrogen; and all other groups are as defined in the Summary of the Invention for a Compound of Formula I. Another embodiment of the invention is directed to a Compound of the Invention where R¹ is halo; R² and R⁴ are hydrogen; R³ is haloalkyl; R⁵ is phenyl substituted with R⁶, R⁷, and R⁸; R⁸ is halo; R⁷ is hydrogen, halo, or alkyl; and R⁶ is —OR¹³ where R¹³ is alkyl substituted with one or two groups independently selected from hydroxy, —C(O)R^{10b}, —NHR^{11a}, —P(O)(OR¹⁶)₂, and —OP(O)(OR¹⁶)₂, and the R¹³ alkyl is additionally optionally substituted with one hydroxy or 1, 2, or 3 halo; or R⁶ is alkyl substituted with one or two R⁹ where each R⁹ is independently hydroxy, —P(O)(OR¹⁶)₂, —OP(O)(OR¹⁶)₂, or —C(O)OR¹⁰; or R⁶ is —NHR^{11a}; or R⁶ is —NHS(O)₂R^{12a} and R^{12a} is alkyl; R¹⁰ is hydrogen; R^{10b} is alkyl, haloalkyl, hydroxyalkyl, carboxyalkyl, or alkyl substituted with one —OP(O)(OR¹⁶)^{2;} R^{11a} is hydrogen, alkyl, or hydroxyalkyl; R^{12a} is alkyl; R¹⁶ is hydrogen; and all other groups are as defined in the Summary of the Invention for a Compound of Formula I.

Another embodiment (R1) of the invention is directed to a compound of Formula I according to Formula I(a):

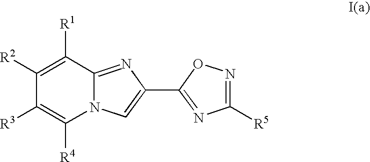

or a single stereoisomer or a mixture of isomers thereof, all optionally as a pharmaceutically acceptable salt thereof wherein R¹, R², R³, R⁴, R⁶, R⁷, and R⁸ are as defined in the Summary of the Invention for a Compound of Formula I or as defined in any of embodiments C, D, E, F, G, G1, H, H1, J, J1-J4, K, K1, K1a-K1m, K2-K10, K10a-K10t, and K11-K13.

Another embodiment (R2) of the invention is directed to a compound of Formula I according to Formula I(b):

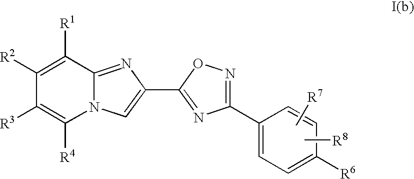

or a single stereoisomer or a mixture of isomers thereof, all optionally as a pharmaceutically acceptable salt thereof wherein R¹, R², R³, R⁴, R⁶, R⁷, and R⁸ are as defined in the Summary of the Invention for a Compound of Formula I or as defined in any of embodiments C, D, E, F, G, G1, J, J1-J4, K, K1, K1a-K1m, K2-K10, K10a-K10t, K11-K13, L1-L6, and M1-M4. Another embodiment of the invention is directed to a Compound of Formula I(b) where R¹, R², R³, R⁴, R⁶, R⁷, and R⁸ are as defined in any of embodiments M1, M2, M3, and M4.

Another embodiment (R3) of the invention is directed to a compound of Formula I according to Formula I(c):

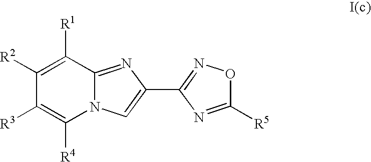

or a single stereoisomer or a mixture of isomers thereof, all optionally as a pharmaceutically acceptable salt thereof wherein R¹, R², R³, R⁴, and R⁵ are as defined in the Summary of the Invention for a Compound of Formula I or as defined in any of embodiments C, D, E, F, G, G1, H, H1, J, J1-J4, K, K1, K1a-K1m, K2-K10, K10a-K10t, and K11-K13.

Another embodiment (R4) of the invention is directed to a compound of Formula I according to Formula I(d):

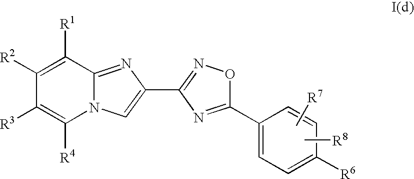

or a single stereoisomer or a mixture of isomers thereof, all optionally as a pharmaceutically acceptable salt thereof wherein R¹, R², R³, R⁴, and R⁵ are as defined in the Summary of the Invention for a Compound of Formula I or as defined in any of embodiments C, D, E, F, G, G1, J, J1-J4, K, K1, K1a-K1m, K2-K10, K10a-K10t, K11-K13, L1-L6, and M1-M4. Another embodiment of the invention is directed to a Compound of Formula I(d) where R¹, R², R³, R⁴, R⁶, R⁷, and R⁸ are as defined in any of embodiments M1, M2, M3, and M4.

Another embodiment (R5) of the invention is directed to a compound of Formula I according to Formula I(e):

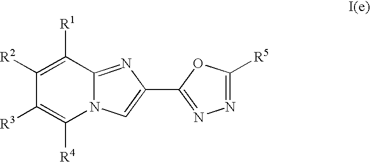

or a single stereoisomer or a mixture of isomers thereof, all optionally as a pharmaceutically acceptable salt thereof wherein R¹, R², R³, R⁴, and R⁵ are as defined in the Summary of the Invention for a Compound of Formula I or as defined in any of embodiments C, D, E, F, G, G1, H, H1, J, J1-J4, K, K1, K1a-K1m, K2-K10, K10a-K10t, and K11-K13.

Another embodiment (R6) of the invention is directed to a compound of Formula I according to Formula I(f):

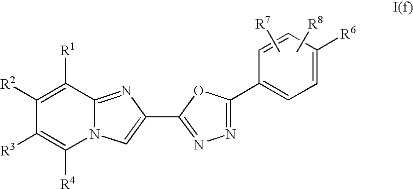

or a single stereoisomer or a mixture of isomers thereof, all optionally as a pharmaceutically acceptable salt thereof wherein R¹, R², R³, R⁴, and R⁵ are as defined in the Summary of the Invention for a Compound of Formula I or as defined in any of embodiments C, D, E, F, G, G1, J, J1-J4, K, K1, K1a-K1m, K2-K10, K10a-K10t, K11-K13, L1-L6, and M1-M4. Another embodiment of the invention is directed to a Compound of Formula I(f) where R¹, R², R³, R⁴, R⁶, R⁷, and R⁸ are as defined in any of embodiments M1, M2, M3, and M4.

Another embodiment (R7) of the invention is directed to a compound of Formula I according to Formula I(g):

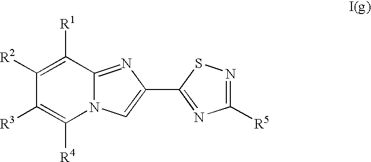

or a single stereoisomer or a mixture of isomers thereof, all optionally as a pharmaceutically acceptable salt thereof wherein R¹, R², R³, R⁴, and R⁵ are as defined in the Summary of the Invention for a Compound of Formula I or as defined in any of embodiments C, D, E, F, G, G1, H, H1, J, J1-J4, K, K1, K1a-K1m, K2-K10, K10a-K10t, and K11-K13.

Another embodiment (R8) of the invention is directed to a compound of Formula I according to Formula I(h):

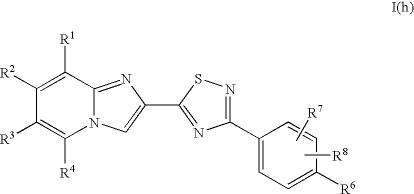

or a single stereoisomer or a mixture of isomers thereof, all optionally as a pharmaceutically acceptable salt thereof wherein R¹, R², R³, R⁴, and R⁵ are as defined in the Summary of the Invention for a Compound of Formula I or as defined in any of embodiments C, D, E, F, G, G1, J, J1-J4, K, K1, K1a-K1m, K2-K10, K10a-K10t, K11-K13, L1-L6, and M1-M4. Another embodiment of the invention is directed to a Compound of Formula I(h) where R¹, R², R³, R⁴, R⁶, R⁷, and R⁸ are as defined in any of embodiments M1, M2, M3, and M4.

Another embodiment (R9) of the invention is directed to a compound of Formula I according to Formula I(j):

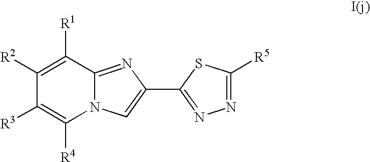

or a single stereoisomer or a mixture of isomers thereof, all optionally as a pharmaceutically acceptable salt thereof wherein R¹, R², R³, R⁴, R⁶, R⁷, and R⁸ are as defined in the Summary of the Invention for a Compound of Formula I or as defined in any of embodiments C, D, E, F, G, G1, H, H1, J, J1-J4, K, K1, K1a-K1m, K2-K10, K10a-K10t, and K11-K13.

Another embodiment (R10) of the invention is directed to a compound of Formula I according to Formula I(k):

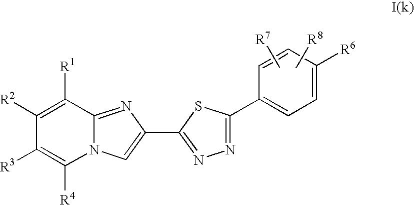

or a single stereoisomer or a mixture of isomers thereof, all optionally as a pharmaceutically acceptable salt thereof wherein R¹, R², R³, R⁴, and R⁵ are as defined in the Summary of the Invention for a Compound of Formula I or as defined in any of embodiments C, D, E, F, G, G1, J, J1-J4, K, K1, K1a-K1m, K2-K10, K10a-K10t, K11-K13, L1-L6, and M1-M4. Another embodiment of the invention is directed to a Compound of Formula I(k) where R¹, R², R³, R⁴, R⁶, R⁷, and R⁸ are as defined in any of embodiments M1, M2, M3, and M4.

Another embodiment (R11) of the invention is directed to a compound of Formula I(m):

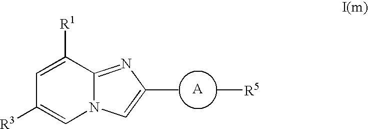

or a single stereoisomer or a mixture of isomers thereof, all optionally as a pharmaceutically acceptable salt thereof wherein R¹, R³,

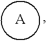

and R⁵ are as defined in the Summary of the Invention for a Compound of Formula I or as defined in any of embodiments 1, A, B, C, E, G, G1, H, H1, J, J1-J4, K, K1, K1a-K1m, K2-K10, K10a-K10t, and K11-K13. Another embodiment of the invention is directed to a Compound of Formula I(m) where R¹ is halo and R³ is haloalkyl; and

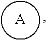

and R⁵ are as defined in the Summary of the Invention for a Compound of Formula I or as defined in any of embodiments 1, A, B, G1, H, H1, J, J1-J4, K, K1, K1a-K1m, K2-K10, K10a-K10t, and K11-K13. Another embodiment of the invention is directed to a Compound of of Formula I(m) where R¹ is halo and R³ is alkoxy; and

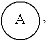

and R⁵ are as defined in the Summary of the Invention for a Compound of Formula I or as defined in any of embodiments 1, A, B, G1, H, H1, J, J1-J4, K, K1, K1a-K1m, K2-K10, K10a-K10t, and K11-K13. Another embodiment of the invention is directed to a Compound of Formula Im where R¹ and R³ are halo; and

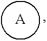

and R⁵ are as defined in the Summary of the Invention for a Compound of Formula I or as defined in any of embodiments 1, A, B, G1, H, H1, J, J1-J4, K, K1, K1a-K1m, K2-K10, K10a-K10t, and K11-K13.

Another embodiment of the Invention (Q1) is directed to a Compound of Formula I where
R¹ is hydrogen, halo, or cyano;
R² is hydrogen, methyl, or methoxy;
R³ is hydrogen, alkyl, alkylsulfonyl, halo, haloalkyl, alkoxy, optionally substituted phenoxy, cyano, alkylsulfonylamino, or nitro;
R⁴ is hydrogen or alkyl;

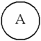

is oxadiazolyl or thiadiazolyl;
R⁵ is phenyl substituted with R⁶, R⁷, and R⁸; or
R⁵ is heteroaryl optionally substituted with one or two R¹⁵ groups independently selected from carboxy; haloalkyl; carboxyalkyl; and alkyl substituted with one —C(O)NR¹⁴R^{14a} group where R¹⁴ is hydrogen, and R^{14a} is hydrogen; provided that when the R⁵ heteroaryl is pyridinyl or thienyl, then the pyridinyl and thienyl is substituted with one R¹⁵ and optionally substituted with a second R¹⁵;
R⁶ is hydroxy; —OR¹³; —NR¹¹R^{11a}; —NR¹²S(O)₂R^{12a}; alkyl substituted with 1, 2, 3, 4, or 5 R⁹ groups; alkenyl optionally substituted with one or two carboxy; or cycloalkyl optionally substituted with 1 or 2 groups independently selected from hydroxyalkyl, carboxy, and —C(O)NR¹⁰R^{10a};
R⁷ and R⁸ are independently hydrogen, halo, haloalkyl, or alkyl;
each R⁹ is independently cyano; hydroxy; halo; —C(O)NR¹⁰R^{10a}; —C(O)OR¹⁰; —NR¹¹R^{11a}; —NR¹²S(O)₂R^{12a}; —P(O)(OR¹⁶)₂; —OP(O)(OR¹⁶)₂; —OS(O)₂OH; —S(O)ₙR¹⁸; or heterocycloalkyl optionally substituted with 1, 2, or 3 groups independently selected from hydroxy, carboxy, alkyl, and hydroxyalkyl;
R¹⁰ is hydrogen or alkyl;
R^{10a} is hydrogen or alkyl;
R^{10b} is hydrogen, alkyl, hydroxyalkyl, carboxyalkyl, haloalkyl, —P(O)(OR¹⁶)₂, —OP(O)(OR¹⁶)₂, or —OS(O)₂OH;
R¹¹ is hydrogen or alkyl;
R^{11a} is hydrogen, alkyl, or alkylsulfonyl;
R¹² is hydrogen or alkyl;
R^{12a} is alkyl;
R¹³ is alkenyl; alkyl optionally substituted with 1, 2, 3, or 4 groups independently selected from halo, hydroxy, alkylsulfonyl, —C(O)OR¹⁰, —OC(O)R^{10b}, —C(O)R^{10b}, —NR¹¹R^{11a}, —P(O)(OR¹⁶)₂, —OP(O)(OR¹⁶)₂, —OS(O)₂OH, and heterocycloalkyl where the heterocycloalkyl is optionally substituted with one, two, or three groups independently selected from alkyl and carboxy;
each R¹⁶ is independently hydrogen or alkyl;
R¹⁸ is alkyl; and
n is 2.

In another embodiment of Q1, A is oxadiazolyl and all other groups are as defined in Q1. In another embodiment of Q1 A is thiadiazolyl and all other groups are as defined in Q1. In another embodiment of Q1, R⁵ is phenyl substituted with R⁶, R⁷, and R⁸ and R⁶, R⁷, R⁸, and all other groups are as defined in Q1. In another embodiment of Q1, R⁵ is according to formula (a), (b), (c), or (d) and all other groups are as defined in Q1. In another embodiment of Q1, R⁵ is according to formula (a) or (d) and R⁸ is halo, haloalkyl, or alkyl and all other groups are as defined in Q1. In another embodiment of Q1, R⁵ is according to formula (b) and all other groups are as defined in Q1. In another embodiment of Q1, R⁵ is according to formula (b) and R⁷ and R⁸ are independently halo, haloalkyl, or alkyl and all other groups are as defined in Q1. In another embodiment of Q1, R¹ is halo or cyano; R³ is alkyl, halo, haloalkyl, alkylsulfonylamino, or alkoxy; and all other groups are as defined Q1. In another embodiment of Q1, R⁵ is phenyl substituted with R⁶, R⁷, and R⁸, R⁶ is —OR¹³; and R⁷, R⁸, R¹³, and all other groups are as defined in Q1. In another embodiment of Q1, R¹ is halo or cyano; R³ is alkyl, halo, haloalkyl, alkylsulfonylamino, or alkoxy; and all other groups are as defined Q1. In another embodiment of Q1, R⁵ is phenyl substituted with R⁶, R⁷, and R⁸, R⁶ is alkyl substituted with 2, 2, or 3 R⁹; and R⁷, R⁸, R⁹, and all other groups are as defined in Q1.

Another embodiment of the Invention (Q3) is directed to a Compound of Formula I
where
R¹ is hydrogen, halo, or cyano;
R² is hydrogen;
R³ is alkyl, halo, haloalkyl, alkylsulfonylamino, or alkoxy;
R⁴ is hydrogen;

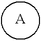

is a 5-membered heteroarylene;
R⁵ is phenyl substituted with R⁶, R⁷, and R⁸;
R⁶ is halo; cyano; —C(O)H; carboxy; alkoxycarbonyl; —C(═NOH)NH₂; —C(O)R¹⁷; —OR¹³; alkyl substituted with 1 or 2 R⁹ groups; alkenyl optionally substituted with one or two alkoxycarbonyl;
R⁷ and R⁸ are independently hydrogen, halo, or alkyl;
each R⁹, when R⁹ is present, is independently cyano; hydroxy; halo; —C(O)H; —C(O)OR¹⁰; —NR11R^{11a}; —S(O)ₙR¹⁸; —C(═NOH)NH₂; —C(═NOH)NH₂; heterocycloalkyl optionally substituted with 1, 2, or 3 groups independently selected from alkyl and alkoxycarbonylamino;
R¹⁰ is alkyl;
R^{10b} is hydrogen or alkyl;
R¹¹ is hydrogen;
R^{11a} is hydrogen or alkoxycarbonyl;
R¹³ is alkenyl; alkyl optionally substituted with 1 or 2 groups independently selected from halo, hydroxy, alkylsulfanyl, cyano, —C(O)OR¹⁰, —C(O)R^{10b}, —NR¹¹R^{11a}, —P(O)(OR¹⁶)₂, —OP(O)(OR¹⁶)₂, —OSi(alkyl)₃, heterocycloalkyl where the heterocycloalkyl is optionally substituted with one alkyl, alkoxycarbonylamino or phenyl;
each R¹⁶ is alkyl;
R¹⁷ is amino or halo;
R¹⁸ is alkyl; and
n is 0.

Another embodiment (N) of the Invention provides a pharmaceutical composition which comprises a compound, or a single stereoisomer or a mixture of isomers thereof, of any one of Formulae I, I(a), I(b), I(c), I(d), Ie, I(f), I(g), I(h), I(j), I(k), and I(m) or any of the above embodiments or a compound selected from Table 1 and 2, all optionally as a pharmaceutically acceptable salt or solvate thereof, and a pharmaceutically acceptable carrier, excipient, or diluent.

Another embodiment (P) of the invention is directed to a method of treating a disease, disorder, or syndrome which method comprises administering to a patient a therapeutically effective amount of a compound, or a single stereoisomer or a mixture of isomers thereof, of Formula I, I(a), I(b), I(c), I(d), I(e), I(f), I(g), I(h), I(j), I(k), or I(m) or any of the above embodiments or a compound selected from Table 1 and 2, all optionally as a pharmaceutically acceptable salt and additionally all optionally as a pharmaceutical composition thereof. In another embodiment of embodiment P, the disease is an autoimmune disease. In another embodiment of embodiment P the autoimmune disease is multiple sclerosis. In another embodiment the autoimmune disease is graft-versus-host disease. In another embodiment, the disease is inflammation caused by an autoimmune disease. In another embodiment of embodiment P the disease is graft versus host disease, multiple sclerosis, rheumatoid arthritis, systemic lupus erythematosis, psoriasis, Grave's disease, myasthenia gravis, Crohn's disease, or ulcerative colitis.

### Representative Compounds

Representative compounds of Formula I are depicted below. The examples are merely illustrative and do not limit the scope of the invention in any way. Compounds of the invention are named according to systematic application of the nomenclature rules agreed upon by the International Union of Pure and Applied Chemistry (IUPAC), International Union of Biochemistry and Molecular Biology (IUBMB), and the Chemical Abstracts Service (CAS). Names were generated using ACD/Labs naming software.

TABLE 1

| Entry | | |
|---|---|---|
| No. | Structure | ACD-generated Name |
| 1 | 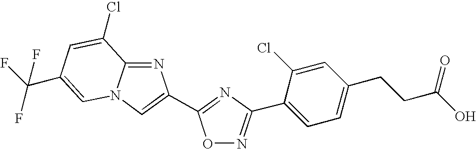 | 3-(3-chloro-4-{5-[8-chloro-6- (trifluoromethyl)imidazo[1,2-a]pyridin-2-yl]- 1,2,4-oxadiazol-3-yl}phenyl)propanoic acid |
| 2 | 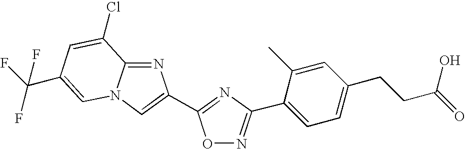 | 3-(4-{5-[8-chloro-6-(trifluoromethyl)imidazo[1,2- a]pyridin-2-yl]-1,2,4-oxadiazol-3-yl}-3- methylphenyl)propanoic acid |
| 3 | 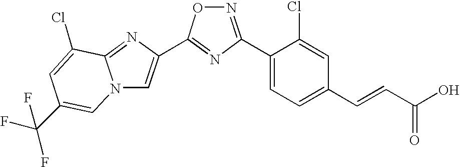 | (2E)-3-(3-chloro-4-{5-[8-chloro-6- (trifluoromethyl)imidazo[1,2-a]pyridin-2-yl]- 1,2,4-oxadiazol-3-yl}phenyl)prop-2-enoic acid |
| 4 | 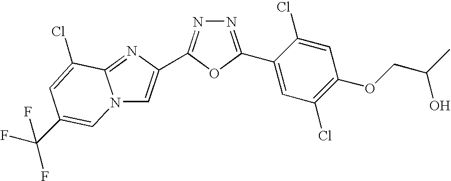 | 1-[(2,5-dichloro-4-{5-[8-chloro-6- (trifluoromethyl)imidazo[1,2-a]pyridin-2-yl]- 1,3,4-oxadiazol-2-yl}phenyl)oxy]propan-2-ol |
| 5 | 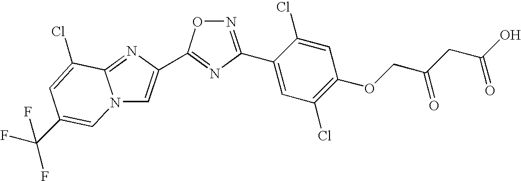 | 4-[(2,5-dichloro-4-{5-[8-chloro-6- (trifluoromethyl)imidazo[1,2-a]pyridin-2-yl]- 1,2,4-oxadiazol-3-yl}phenyl)oxy]-3-oxobutanoic acid |
| 6 | 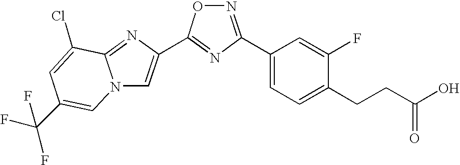 | 3-(4-{5-[8-chloro-6-(trifluoromethyl)imidazo[1,2- a]pyridin-2-yl]-1,2,4-oxadiazol-3-yl}-2- fluorophenyl)propanoic acid |
| 7 | 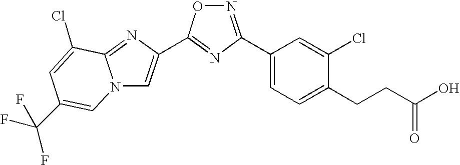 | 3-(2-chloro-4-{5-[8-chloro-6- (trifluoromethyl)imidazo[1,2-a]pyridin-2-yl]- 1,2,4-oxadiazol-3-yl}phenyl)propanoic acid |
| 8 | 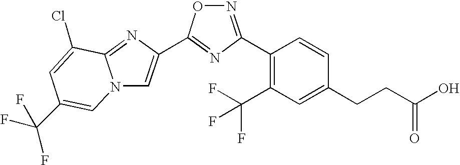 | 3-[4-{5-[8-chloro-6-(trifluoromethyl)imidazo[1,2- a]pyridin-2-yl]-1,2,4-oxadiazol-3-yl}-3- (trifluoromethyl)phenyl]propanoic acid |
| 9 | 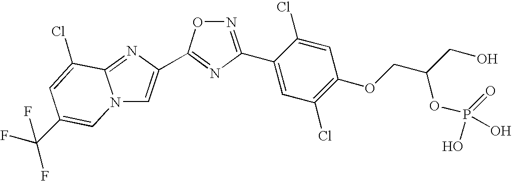 | 2-[(2,5-dichloro-4-{5-[8-chloro-6- (trifluoromethyl)imidazo[1,2-a]pyridin-2-yl]- 1,2,4-oxadiazol-3-yl}phenyl)oxy]-1- (hydroxymethyl)ethyl dihydrogen phosphate |
| 10 | 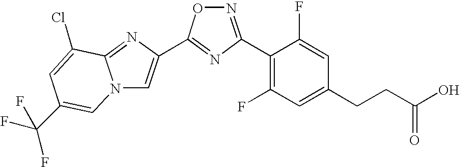 | 3-(4-{5-[8-chloro-6-(trifluoromethyl)imidazo[1,2- a]pyridin-2-yl]-1,2,4-oxadiazol-3-yl}-3,5- difluorophenyl)propanoic acid |
| 11 | 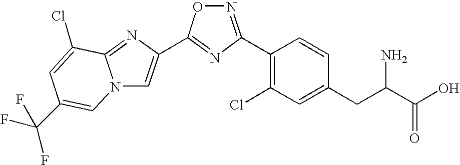 | 3-chloro-4-{5-[8-chloro-6- (trifluoromethyl)imidazo[1,2-a]pyridin-2-yl]- 1,2,4-oxadiazol-3-yl}phenylalanine |
| 12 | 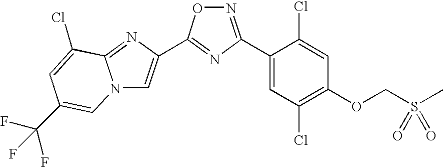 | 8-chloro-2-[3-(2,5-dichloro-4- {[(methylsulfonyl)methyl]oxy}phenyl)-1,2,4- oxadiazol-5-yl]-6-(trifluoromethyl)imidazo[1,2- a]pyridine |
| 13 | 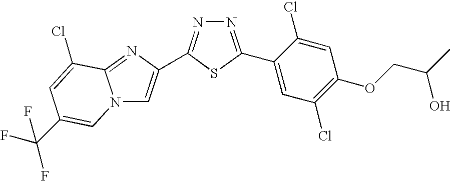 | 1-[(2,5-dichloro-4-{5-[8-chloro-6- (trifluoromethyl)imidazo[1,2-a]pyridin-2-yl]- 1,3,4-thiadiazol-2-yl}phenyl)oxy]propan-2-ol |
| 14 | 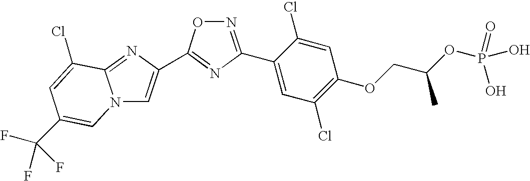 | (1S)-2-[(2,5-dichloro-4-{5-[8-chloro-6- (trifluoromethyl)imidazo[1,2-a]pyridin-2-yl]- 1,2,4-oxadiazol-3-yl}phenyl)oxy]-1-methylethyl dihydrogen phosphate |
| 15 | 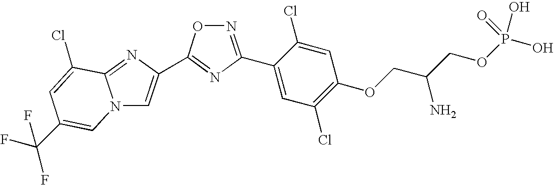 | 2-amino-3-[(2,5-dichloro-4-{5-[8-chloro-6- (trifluoromethyl)imidazo[1,2-a]pyridin-2-yl]- 1,2,4-oxadiazol-3-yl}phenyl)oxy]propyl dihydrogen phosphate |
| 16 | 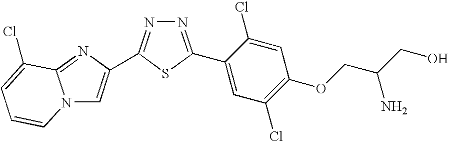 | 2-amino-3-({5-chloro-4-[5-(8-chloroimidazo[1,2- a]pyridin-2-yl)-1,3,4-thiadiazol-2-yl]-2- fluorophenyl}oxy)propan-1-ol |
| 17 | 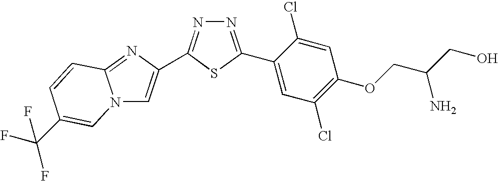 | 2-amino-3-[(5-chloro-2-fluoro-4-{5-[6- (trifluoromethyl)imidazo[1,2-a]pyridin-2-yl]- 1,3,4-thiadiazol-2-yl}phenyl)oxy]propan-1-ol |
| 18 | 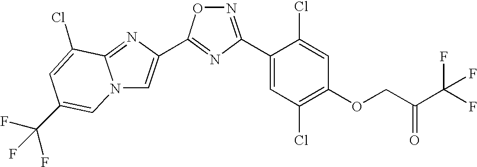 | 3-[(2,5-dichloro-4-{5-[8-chloro-6- (trifluoromethyl)imidazo[1,2-a]pyridin-2-yl]- 1,2,4-oxadiazol-3-yl}phenyl)oxy]-1,1,1- trifluoropropan-2-one |
| 19 | 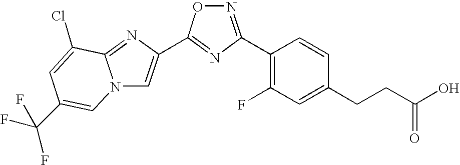 | 3-(4-{5-[8-chloro-6-(trifluoromethyl)imidazo[1,2- a]pyridin-2-yl]-1,2,4-oxadiazol-3-yl}-3- fluorophenyl)propanoic acid |
| 20 | 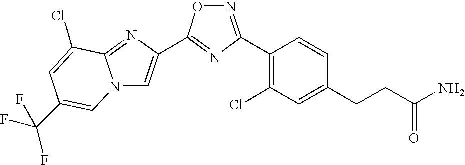 | 3-(3-chloro-4-{5-[8-chloro-6- (trifluoromethyl)imidazo[1,2-a]pyridin-2-yl]- 1,2,4-oxadiazol-3-yl}phenyl)propanamide |
| 21 | 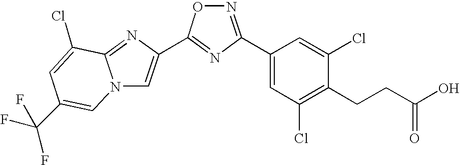 | 3-(2,6-dichloro-4-{5-[8-chloro-6- (trifluoromethyl)imidazo[1,2-a]pyridin-2-yl]- 1,2,4-oxadiazol-3-yl}phenyl)propanoic acid |
| 22 | 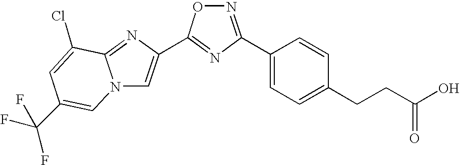 | 3-(4-{5-[8-chloro-6-(trifluoromethyl)imidazo[1,2- a[pyridin-2-yl]-1,2,4-oxadiazol-3- yl}phenyl)propanoic acid |
| 23 | 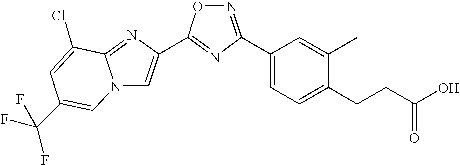 | 3-(4-{5-[8-chloro-6-(trifluoromethyl)imidazo[1,2- a]pyridin-2-yl]-1,2,4-oxadiazol-3-yl}-2- methylphenyl)propanoic acid |
| 24 | 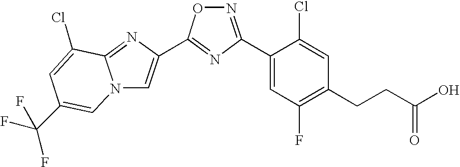 | 3-(5-chloro-4-{5-[8-chloro-6- (trifluoromethyl)imidazo[1,2-a]pyridin-2-yl]- 1,2,4-oxadiazol-3-yl}-2-fluorophenyl)propanoic acid |
| 25 | 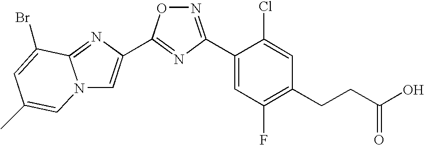 | 3-{4-[5-(8-bromo-6-methylimidazo[1,2-a]pyridin- 2-yl)-1,2,4-oxadiazol-3-yl]-5-chloro-2- fluorophenyl}propanoic acid |
| 26 | 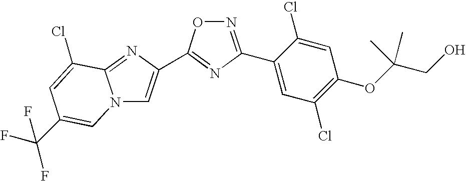 | 2-[(2,5-dichloro-4-{5-[8-chloro-6- (trifluoromethyl)imidazo[1,2-a]pyridin-2-yl]- 1,2,4-oxadiazol-3-yl}phenyl)oxy]-2- methylpropan-1-ol |
| 27 | 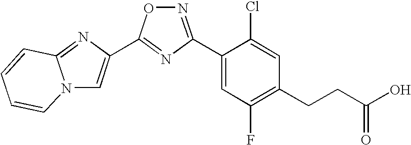 | 3-[5-chloro-2-fluoro-4-(5-imidazo[1,2-a]pyridin- 2-yl-1,2,4-oxadiazol-3-yl)phenyl]propanoic acid |
| 28 | 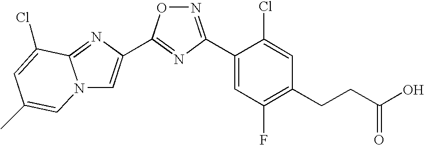 | 3-{5-chloro-4-[5-(8-chloro-6-methylimidazo[1,2- a]pyridin-2-yl)-1,2,4-oxadiazol-3-yl]-2- fluorophenyl}propanoic acid |
| 29 | 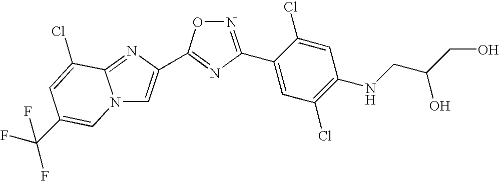 | 3-[(2,5-dichloro-4-{5-[8-chloro-6- (trifluoromethyl)imidazo[1,2-a]pyridin-2-yl]- 1,2,4-oxadiazol-3-yl}phenyl)amino]propane-1,2- diol |
| 30 | 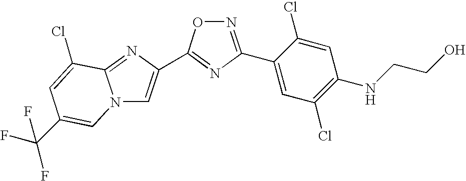 | 2-[(2,5-dichloro-4-{5-[8-chloro-6- (trifluoromethyl)imidazo[1,2-a]pyridin-2-yl]- 1,2,4-oxadiazol-3-yl}phenyl)amino]ethanol |
| 31 | 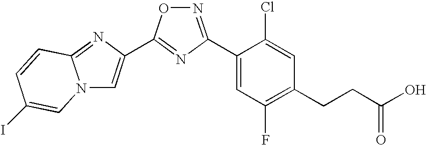 | 3-{5-chloro-2-fluoro-4-[5-(6-iodoimidazo[1,2- a]pyridin-2-yl)-1,2,4-oxadiazol-3- yl]phenyl}propanoic acid |
| 32 | 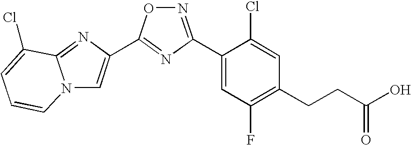 | 3-{5-chloro-4-[5-(8-chloroimidazo[1,2-a]pyridin- 2-yl)-1,2,4-oxadiazol-3-yl]-2- fluorophenyl}propanoic acid |
| 33 | 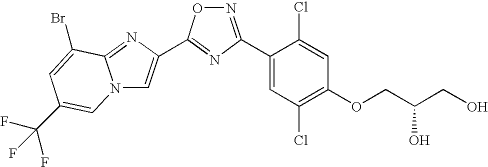 | (25)-3-[(4-{5-[8-bromo-6- (trifluoromethyl)imidazo[1,2-a]pyridin-2-yl]- 1,2,4-oxadiazol-3-yl}-2,5- dichlorophenyl)oxy]propane-1,2-diol |
| 34 | 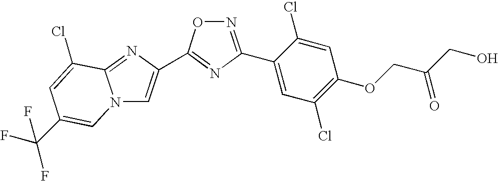 | 1-[(2,5-dichloro-4-{5-[8-chloro-6- (trifluoromethyl)imidazo[1,2-a]pyridin-2-yl]- 1,2,4-oxadiazol-3-yl}phenyl)oxy]-3- hydroxypropan-2-one |
| 35 | 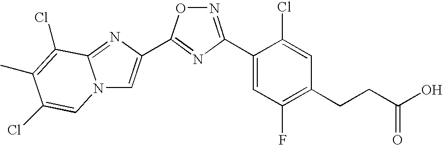 | 3-{5-chloro-4-[5-(6,8-dichloro-7- methylimidazo[1,2-a]pyridin-2-yl)-1,2,4- oxadiazol-3-yl]-2-fluorophenyl}propanoic acid |
| 36 | 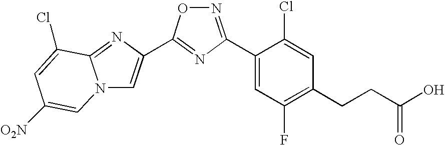 | 3-{5-chloro-4-[5-(8-chloro-6-nitroimidazo[1,2- a]pyridin-2-yl)-1,2,4-oxadiazol-3-yl]-2- fluorophenyl}propanoic acid |
| 37 | 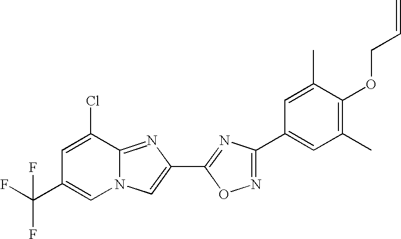 | 3-(4-(allyloxy)-3,5-dimethylphenyl)-5-(8-chloro- 6-(trifluoromethyl)imidazo[1,2-a]pyridin-2-yl)- 1,2,4-oxadiazole |
| 38 | 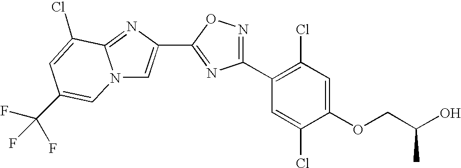 | (2S)-1-[(2,5-dichloro-4-{5-[8-chloro-6- (trifluoromethyl)imidazo[1,2-a]pyridin-2-yl]- 1,2,4-oxadiazol-3-yl}phenyl)oxy]propan-2-ol |
| 39 | 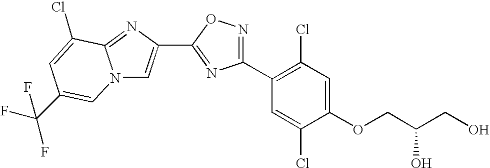 | (2S)-3-[(2,5-dichloro-4-{5-[8-chloro-6- (trifluoromethyl)imidazo[1,2-a]pyridin-2-yl]- 1,2,4-oxadiazol-3-yl}phenyl)oxy]propane-1,2-diol |
| 40 | 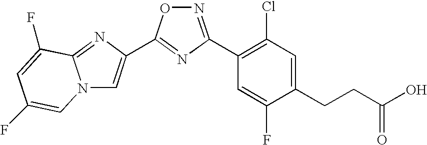 | 3-{5-chloro-4-[5-(6,8-difluoroimidazo[1,2- a]pyridin-2-yl)-1,2,4-oxadiazol-3-yl]-2- fluorophenyl}propanoic acid |
| 41 | 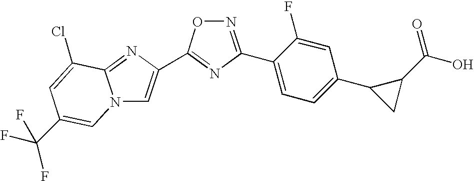 | 2-(4-{5-[8-chloro-6-(trifluoromethyl)imidazo[1,2- a]pyridin-2-yl]-1,2,4-oxadiazol-3-yl}-3- fluorophenyl)cyclopropanecarboxylic acid |
| 42 | 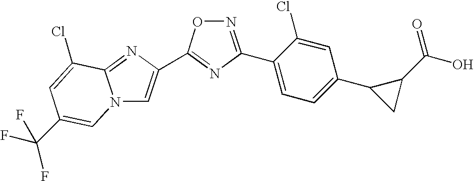 | 2-(3-chloro-4-{5-[8-chloro-6- (trifluoromethyl)imidazo[1,2-a]pyridin-2-yl]- 1,2,4-oxadiazol-3- yl}phenyl)cyclopropanecarboxylic acid |
| 43 | 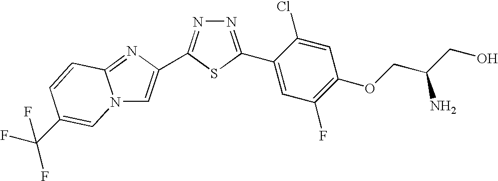 | (2R)-2-amino-3-[(5-chloro-2-fluoro-4-{5-[6- (trifluoromethyl)imidazo[1,2-a]pyridin-2-yl]- 1,3,4-thiadiazol-2-yl}phenyl)oxy]propan-1-ol |
| 44 | 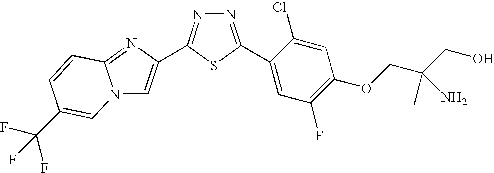 | 2-amino-3-[(5-chloro-2-fluoro-4-{5-[6- (trifluoromethyl)imidazo[1,2-a]pyridin-2-yl]- 1,3,4-thiadiazol-2-yl}phenyl)oxy]-2- methylpropan-1-ol |
| 45 | 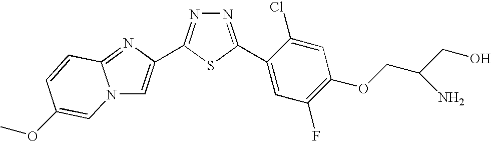 | 2-amino-3-[(5-chloro-2-fluoro-4-{5-[6- (methyloxy)imidazo[1,2-a]pyridin-2-yl]-1,3,4- thiadiazol-2-yl}phenyl)oxy]propan-1-ol |
| 46 | 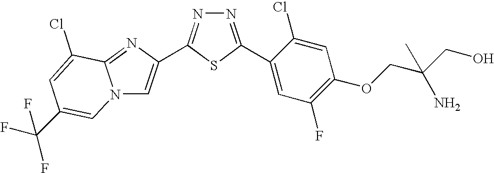 | 2-amino-3-[(5-chloro-4-{5-[8-chloro-6- (trifluoromethyl)imidazo[1,2-a]pyridin-2-yl]- 1,3,4-thiadiazol-2-yl}-2-fluorophenyl)oxy]-2- methylpropan-1-ol |
| 47 | 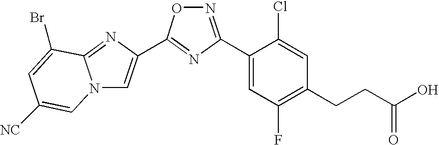 | 3-{4-[5-(8-bromo-6-cyanoimidazo[1,2-a]pyridin- 2-yl)-1,2,4-oxadiazol-3-yl]-5-chloro-2- fluorophenyl}propanoic acid |
| 48 | 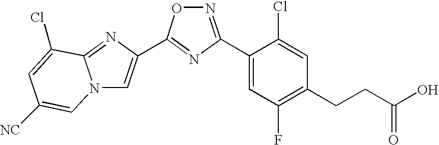 | 3-{5-chloro-4-[5-(8-chloro-6-cyanoimidazo[1,2- a]pyridin-2-yl)-1,2,4-oxadiazol-3-yl]-2- fluorophenyl}propanoic acid |
| 49 | 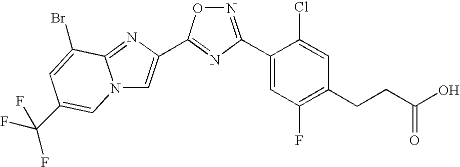 | 3-(4-{5-[8-bromo-6-(trifluoromethyl)imidazo[1,2- a]pyridin-2-yl]-1,2,4-oxadiazol-3-yl}-5-chloro-2- fluorophenyl)propanoic acid |
| 50 | 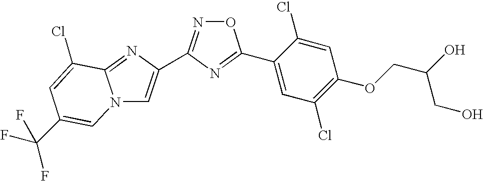 | 3-[(2,5-dichloro-4-{3-[8-chloro-6- (trifluoromethyl)imidazo[1,2-a]pyridin-2-yl]- 1,2,4-oxadiazol-5-yl}phenyl)oxy]propane-1,2-diol |
| 51 | 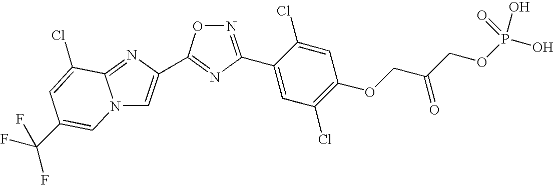 | 3-[(2,5-dichloro-4-{5-[8-chloro-6- (trifluoromethyl)imidazo[1,2-a]pyridin-2-yl]- 1,2,4-oxadiazol-3-yl}phenyl)oxy]-2-oxopropyl dihydrogen phosphate |
| 52 | 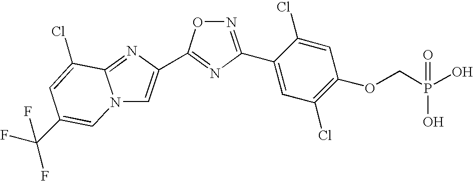 | {[(2,5-dichloro-4-{5-[8-chloro-6- (trifluoromethyl)imidazo[1,2-a]pyridin-2-yl]- 1,2,4-oxadiazol-3- yl}phenyl)oxy]methyl}phosphonic acid |
| 53 | 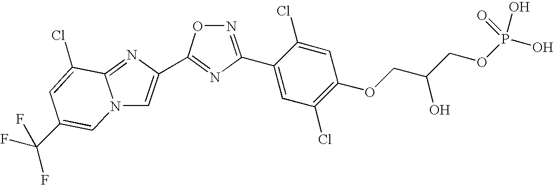 | 3-[(2,5-dichloro-4-{5-[8-chloro-6- (trifluoromethyl)imidazo[1,2-a]pyridin-2-yl]- 1,2,4-oxadiazol-3-yl}phenyl)oxy]-2- hydroxypropyl dihydrogen phosphate |
| 54 | 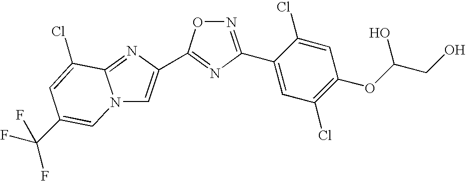 | 3-(2,5-dichloro-4-{5-[8-chloro-6- (trifluoromethyl)imidazo[1,2-a]pyridin-2-yl]- 1,2,4-oxadiazol-3-yl}phenyl)propane-1,2-diol |
| 55 | 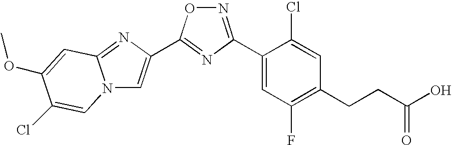 | 3-(5-chloro-4-{5-[6-chloro-7- (methyloxy)imidazo[1,2-a]pyridin-2-yl]-1,2,4- oxadiazol-3-yl}-2-fluorophenyl)propanoic acid |
| 56 | 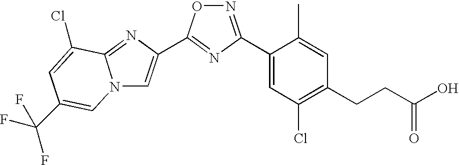 | 3-(2-chloro-4-{5-[8-chloro-6- (trifluoromethyl)imidazo[1,2-a]pyridin-2-yl]- 1,2,4-oxadiazol-3-yl}-5-methylphenyl)propanoic acid |
| 57 | 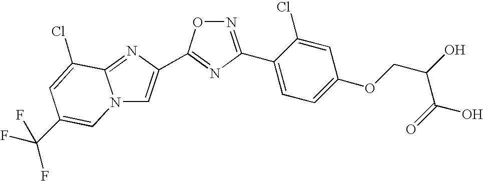 | 3-[(3-chloro-4-{5-[8-chloro-6- (trifluoromethyl)imidazo[1,2-a]pyridin-2-yl]- 1,2,4-oxadiazol-3-yl}phenyl)oxy]-2- hydroxypropanoic acid |
| 58 | 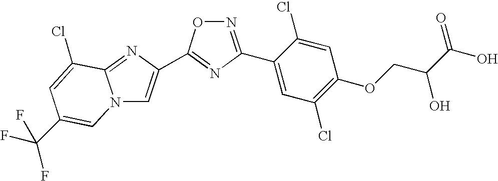 | 3-[(2,5-dichloro-4-{5-[8-chloro-6- (trifluoromethyl)imidazo[1,2-a]pyridin-2-yl]- 1,2,4-oxadiazol-3-yl}phenyl)oxy]-2- hydroxypropanoic acid |
| 59 | 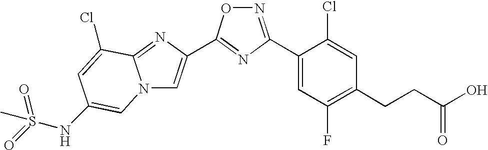 | 3-[5-chloro-4-(5-{8-chloro-6- [(methylsulfonyl)amino]imidazo[1,2-a]pyridin-2- yl}-1,2,4-oxadiazol-3-yl)-2- fluorophenyl]propanoic acid |
| 60 | 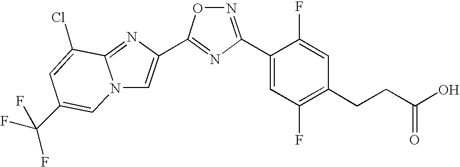 | 3-(4-{5-[8-chloro-6-(trifluoromethyl)imidazo[1,2- a]pyridin-2-yl]-1,2,4-oxadiazol-3-yl}-2,5- difluorophenyl)propanoic acid |
| 61 | 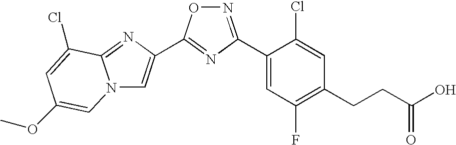 | 3-(5-chloro-4-{5-[8-chloro-6- (methyloxy)imidazo[1,2-a]pyridin-2-yl]-1,2,4- oxadiazol-3-yl}-2-fluorophenyl)propanoic acid |
| 62 | 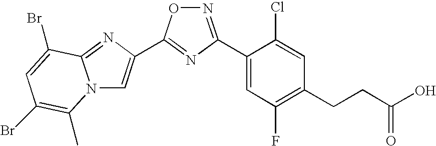 | 3-{5-chloro-4-[5-(6,8-dibromo-5- methylimidazo[1,2-a]pyridin-2-yl)-1,2,4- oxadiazol-3-yl]-2-fluorophenyl}propanoic acid |
| 63 | 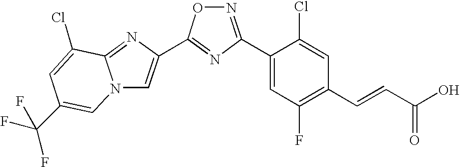 | (2E)-3-(5-chloro-4-{5-[8-chloro-6- (trifluoromethyl)imidazo[1,2-a]pyridin-2-yl]- 1,2,4-oxadiazol-3-yl}-2-fluorophenyl)prop-2- enoic acid |
| 64 | 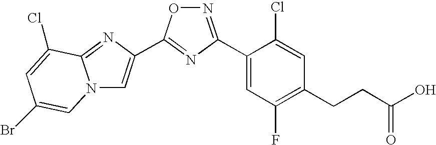 | 3-{4-[5-(6-bromo-8-chloroimidazo[1,2-a]pyridin- 2-yl)-1,2,4-oxadiazol-3-yl]-5-chloro-2- fluorophenyl}propanoic acid |
| 65 | 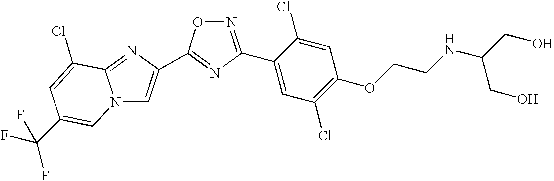 | 2-({2-[(2,5-dichloro-4-{5-[8-chloro-6- (trifluoromethyl)imidazo[1,2-a]pyridin-2-yl]- 1,2,4-oxadiazol-3- yl}phenyl)oxy]ethyl}amino)propane-1,3-diol |
| 66 | 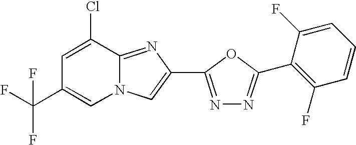 | 8-chloro-2-[5-(2,6-difluorophenyl)-1,3,4- oxadiazol-2-yl]-6-(trifluoromethyl)imidazo[1,2- a]pyridine |
| 67 | 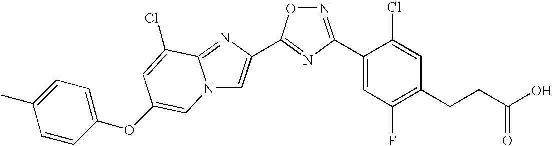 | 3-[5-chloro-4-(5-{8-chloro-6-[(4- methylphenyl)oxy]imidazo[1,2-a]pyridin-2-yl}- 1,2,4-oxadiazol-3-yl)-2-fluorophenyl]propanoic acid |
| 68 | 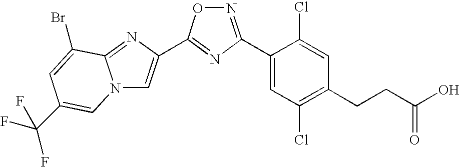 | 3-(4-{5-[8-bromo-6-(trifluoromethyl)imidazo[1,2- a]pyridin-2-yl]-1,2,4-oxadiazol-3-yl}-2,5- dichlorophenyl)propanoic acid |
| 69 | 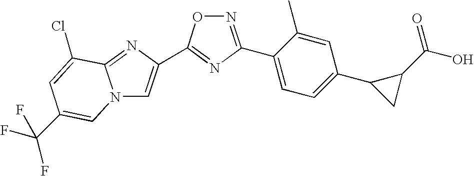 | 2-(4-{5-[8-chloro-6-(trifluoromethyl)imidazo[1,2- a]pyridin-2-yl]-1,2,4-oxadiazol-3-yl}-3- methylphenyl)cyclopropanecarboxylic acid |
| 70 | 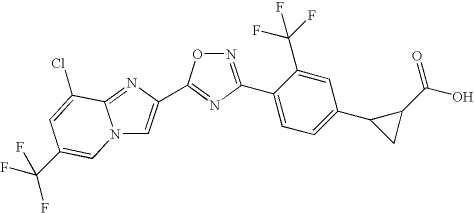 | 2-[4-{5-[8-chloro-6-(trifluoromethyl)imidazo[1,2- a]pyridin-2-yl]-1,2,4-oxadiazol-3-yl}-3- (trifluoromethyl)phenyl]cyclopropanecarboxylic acid |
| 71 | 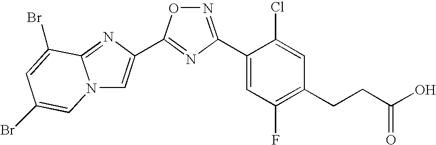 | 3-{5-chloro-4-[5-(6,8-dibromoimidazo[1,2- a]pyridin-2-yl)-1,2,4-oxadiazol-3-yl]-2- fluorophenyl}propanoic acid |
| 72 | 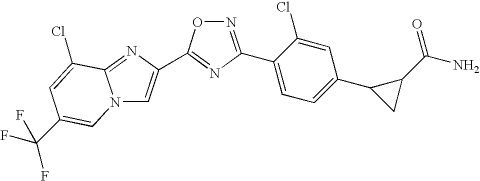 | 2-(3-chloro-4-{5-[8-chloro-6- (trifluoromethyl)imidazo[1,2-a]pyridin-2-yl]- 1,2,4-oxadiazol-3- yl}phenyl)cyclopropanecarboxamide |
| 73 | 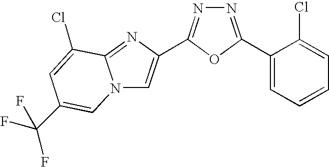 | 8-chloro-2-[5-(2-chlorophenyl)-1,3,4-oxadiazol-2- yl]-6-(trifluoromethyl)imidazo[1,2-a]pyridine |
| 74 | 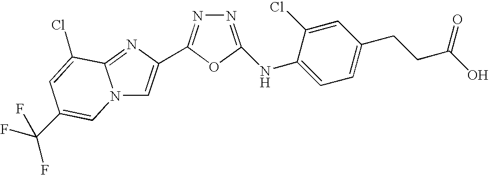 | 3-[3-chloro-4-({5-[8-chloro-6- (trifluoromethyl)imidazo[1,2-a]pyridin-2-yl]- 1,3,4-oxadiazol-2-yl}amino)phenyl]propanoic acid |
| 75 | 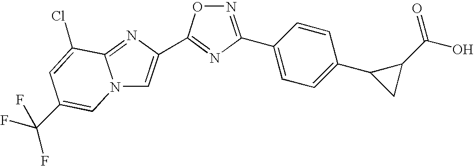 | 2-(4-{5-[8-chloro-6-(trifluoromethyl)imidazo]1,2- a]pyridin-2-yl]-1,2,4-oxadiazol-3- yl}phenyl)cyclopropanecarboxylic acid |
| 76 | 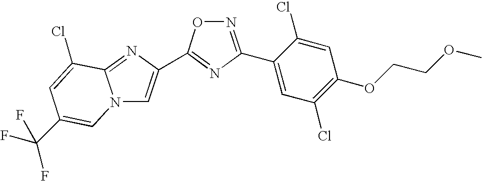 | 8-chloro-2-[3-(2,5-dichloro-4-{[2- (methyloxy)ethyl]oxy}phenyl)-1,2,4-oxadiazol-5- yl]-6-(trifluoromethyl)imidazo[1,2-a]pyridine |
| 77 | 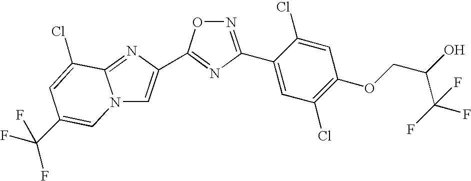 | 3-[(2,5-dichloro-4-{5-[8-chloro-6- (trifluoromethyl)imidazo[1,2-a]pyridin-2-yl]- 1,2,4-oxadiazol-3-yl}phenyl)oxy]-1,1,1- trifluoropropan-2-ol |
| 78 | 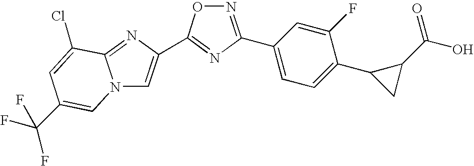 | 2-(4-{5-[8-chloro-6-(trifluoromethyl)imidazo[1,2- a]pyridin-2-yl]-1,2,4-oxadiazol-3-yl}-2- fluorophenyl)cyclopropanecarboxylic acid |
| 79 | 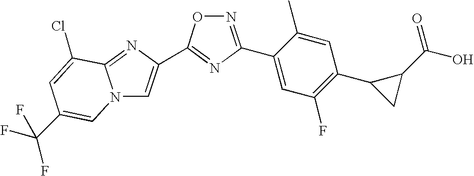 | 2-(4-{5-[8-chloro-6-(trifluoromethyl)imidazo[1,2- a]pyridin-2-yl]-1,2,4-oxadiazol-3-yl}-2-fluoro-5- methylphenyl)cyclopropanecarboxylic acid |
| 80 | 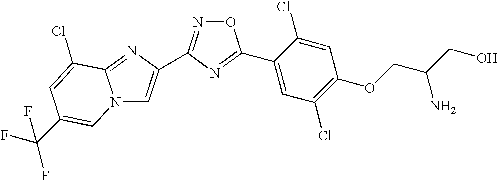 | 2-amino-3-[(2,5-dichloro-4-{3-[8-chloro-6- (trifluoromethyl)imidazo[1,2-a]pyridin-2-yl]- 1,2,4-oxadiazol-5-yl}phenyl)oxy]propan-1-ol |
| 81 | 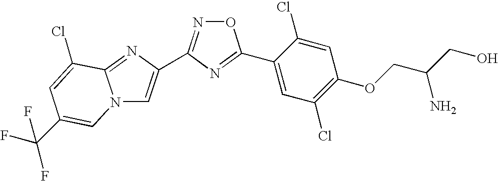 | 2-amino-3-[(5-chloro-4-{3-[8-chloro-6- (trifluoromethyl)imidazo[1,2-a]pyridin-2-yl]- 1,2,4-oxadiazol-5-yl}-2-fluorophenyl)oxy]propan- 1-ol |
| 82 | 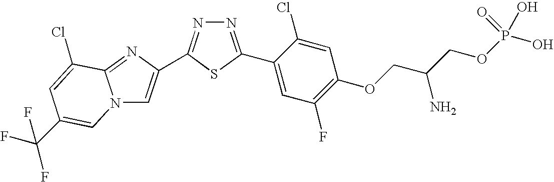 | 2-amino-3-[(5-chloro-4-{5-[8-chloro-6- (trifluoromethyl)imidazo[1,2-a]pyridin-2-yl]- 1,3,4-thiadiazol-2-yl}-2-fluorophenyl)oxy]propyl dihydrogen phosphate |
| 83 | 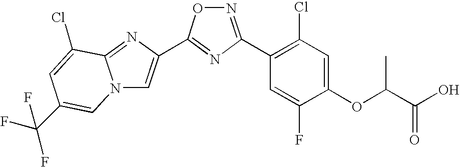 | 2-[(5-chloro-4-{5-[8-chloro-6- (trifluoromethyl)imidazo[1,2-a]pyridin-2-yl]- 1,2,4-oxadiazol-3-yl}-2- fluorophenyl)oxy]propanoic acid |
| 84 | 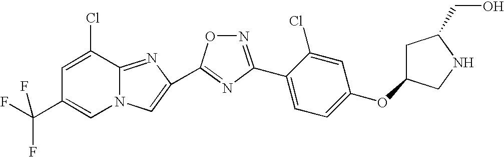 | {(2R,4S)-4-[(3-chloro-4-{5-[8-chloro-6- (trifluoromethyl)imidazo[1,2-a]pyridin-2-yl]- 1,2,4-oxadiazol-3-yl}phenyl)oxy]pyrrolidin-2- yl}methanol |
| 85 | 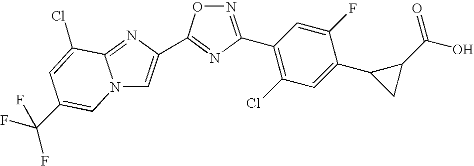 | 2-(5-chloro-4-{5-[8-chloro-6- (trifluoromethyl)imidazo[1,2-a]pyridin-2-yl]- 1,2,4-oxadiazol-3-yl}-2- fluorophenyl)cyclopropanecarboxylic acid |
| 86 | 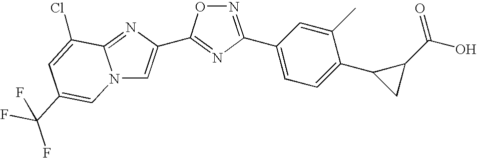 | 2-(4-{5-[8-chloro-6-(trifluoromethyl)imidazo[1,2- a]pyridin-2-yl]-1,2,4-oxadiazol-3-yl}-2- methylphenyl)cyclopropanecarboxylic acid |
| 87 | 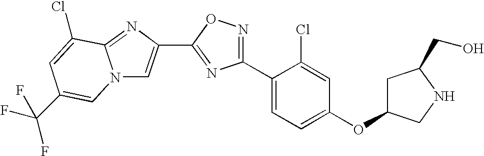 | {(2S,4S)-4-[(3-chloro-4-{5-[8-chloro-6- (trifluoromethyl)imidazo[1,2-a]pyridin-2-yl]- 1,2,4-oxadiazol-3-yl}phenyl)oxy]pyrrolidin-2- yl}methanol |
| 88 | 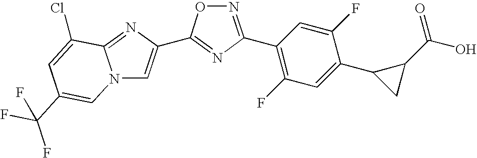 | 2-(4-{5-[8-chloro-6-(trifluoromethyl)imidazo[1,2- a]pyridin-2-yl]-1,2,4-oxadiazol-3-yl}-2,5- difluorophenyl)cyclopropanecarboxylic acid |
| 89 | 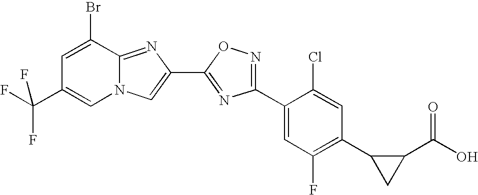 | 2-(4-{5-[8-bromo-6-(trifluoromethyl)imidazo[1,2- a]pyridin-2-yl]-1,2,4-oxadiazol-3-yl}-5-chloro-2- fluorophenyl)cyclopropanecarboxylic acid |
| 90 | 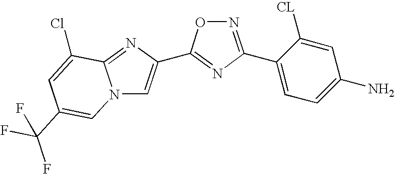 | 3-chloro-4-{5-[8-chloro-6- (trifluoromethyl)imidazo[1,2-a]pyridin-2-yl]- 1,2,4-oxadiazol-3-yl}aniline |
| 91 | 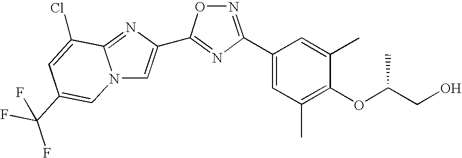 | (2R)-2-[(4-{5-[8-chloro-6- (trifluoromethyl)imidazo[1,2-a]pyridin-2-yl]- 1,2,4-oxadiazol-3-yl}-2,6- dimethylphenyl)oxy]propan-1-ol |
| 92 | 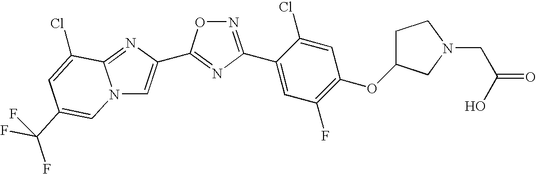 | {3-[(5-chloro-4-{5-[8-chloro-6- (trifluoromethyl)imidazo[1,2-a]pyridin-2-yl]- 1,2,4-oxadiazol-3-yl}-2- fluorophenyl)oxy]pyrrolodin-1-yl}acetic acid |
| 93 | 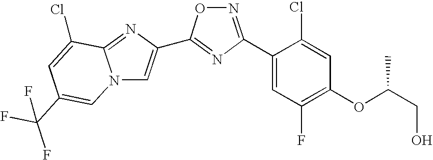 | (2R)-2-[(5-chloro-4-{5-[8-chloro-6- (trifluoromethyl)imidazo[1,2-a]pyridin-2-yl]- 1,2,4-oxadiazol-3-yl}-2-fluorophenyl)oxy]propan- 1-ol |
| 94 | 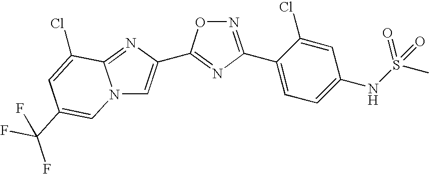 | N-(3-chloro-4-{5-[8-chloro-6- (trifluoromethyl)imidazo[1,2-a]pyridin-2-yl]- 1,2,4-oxadiazol-3-yl}phenyl)methanesulfonamide |
| 95 | 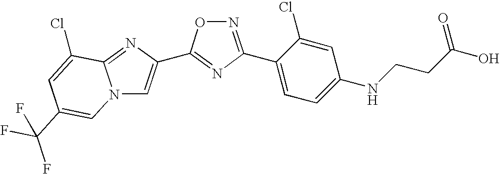 | N-(3-chloro-4-{5-[8-chloro-6- (trifluoromethyl)imidazo[1,2-a]pyridin-2-yl]- 1,2,4-oxadiazol-3-yl}phenyl)-beta-alanine |
| 96 | 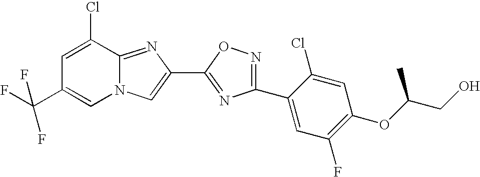 | (2S)-2-[(5-chloro-4-{5-[8-chloro-6- (trifluoromethyl)imidazo[1,2-a]pyridin-2-yl]- 1,2,4-oxadiazol-3-yl}-2-fluorophenyl)oxy]propan- 1-ol |
| 97 | 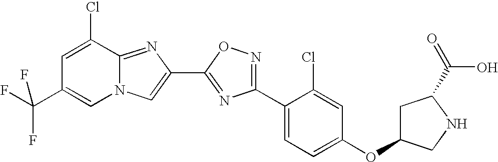 | (4S)-4-[(3-chloro-4-{5-[8-chloro-6- (trifluoromethyl)imidazo[1,2-a]pyridin-2-yl]- 1,2,4-oxadiazol-3-yl}phenyl)oxy]-D-proline |
| 98 | 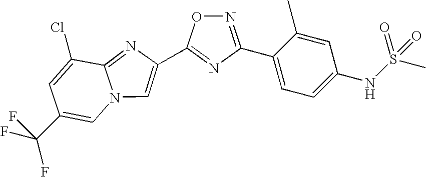 | N-(4-{5-[8-chloro-6- (trifluoromethyl)imidazo[1,2-a]pyridin-2-yl]- 1,2,4-oxadiazol-3-yl}-3- methylphenyl)methane sulfonamide |
| 99 | 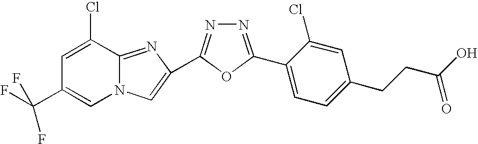 | 3-(3-chloro-4-{5-[8-chloro-6 (trifluoromethyl)imidazo[1,2-a]pyridin-2-yl]- 1,3,4-oxadiazol-2-yl}phenyl)propanoic acid |
| 100 | 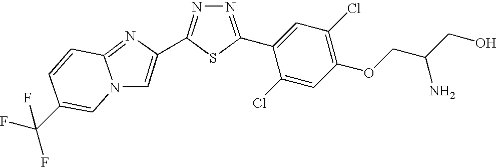 | 2-amino-3-[(2,5;dichloro-4-{5-[6- (trifluomethyl)imidazo[1,2-a]pyridin-2-yl] 1,3,4-thiadiazol-2-yl}phenyl)oxy]propan-1-ol |
| 101 | 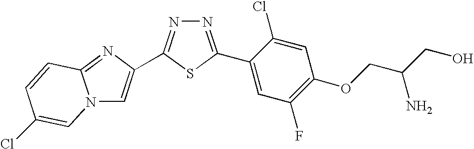 | 2-amino-3-({5-chloro-4-[5-(6-chloroimidazo[1,2- a]pyridin-2-yl)-1,3,4-thiadiazol-2-yl]-2- fluorophenyl}oxy)propan-1-ol |
| 102 | 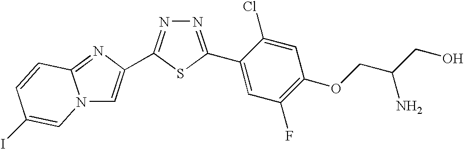 | 2-amino-3-({5-chloro-2-fluoro-4-[5-(6- iodoimidazo[1,2-a]pyridin-2-yl)-1,3,4-thiadiazol- 2-yl]phenyl}oxy)propan-1-ol |
| 103 | 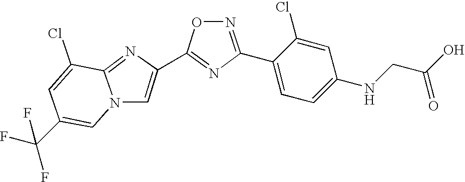 | N-(3-chloro-4-{5-[8-chloro-6- (trifluormethyl)imidazo[1,2-a]pyridin-2-yl]- 1,2,4-oxadiazol-3-yl}phenyl)glycine |
| 104 | 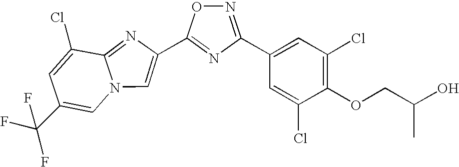 | 1-[(2,6-dichloro-4-{5-[8-chloro-6- (trifluoromethyl)imidazo[1,2-a]pyridin-2-yl]- 1,2,4-oxadiazol-3-yl}phenyl)oxy]propan-2-ol |
| 105 | 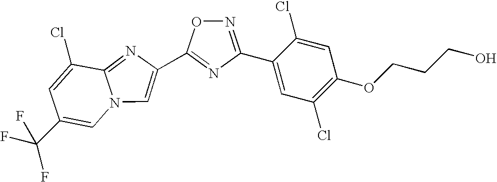 | 3-[(2,5-dichloro-4-{5-[8-chloro-6- (trifluoromethyl)imidazo[1,2-a]pyridin-2-yl]- 1,2,4-oxadiazol-3-yl}phenyl)oxy]propan-1-ol |
| 106 | 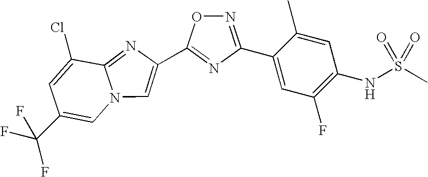 | N-(4-{5-[8-chloro-6- (trifluoromethyl)imidazo[1,2-a]pyridin-2-yl]- 1,2,4-oxadiazol-3-yl}-2-fluoro-5- methylphenyl)methane sulfonamide |
| 107 | 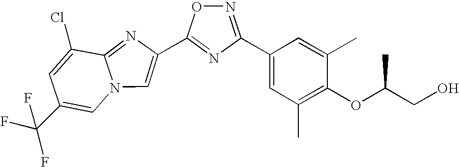 | (2S)-2-[(4-{5-[8-chloro-6- (trifluoromethyl)imidazo[1,2-a]pyridin-2-yl]- 1,2,4-oxadiazol-3-yl}-2,6- dimethylphenyl)oxy]propan-1-ol |
| 108 | 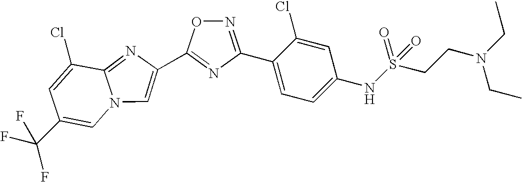 | N-(3-chloro-4-{5-[8-chloro-6- (trifluoromethyl)imidazo[1,2-a]pyridin-2-yl]- 1,2,4-oxadiazol-3-yl}phenyl)-2- (diethylamino)ethanesulfonamide |
| 109 | 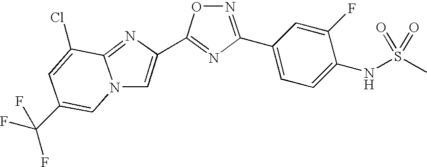 | N-(4-{5-[8-chloro-6- (trifluoromethyl)imidazo[1,2-a]pyridin-2-yl]- 1,2,4-oxadiazol-3-yl}-2- fluorophenyl)methanesulfonamide |
| 110 | 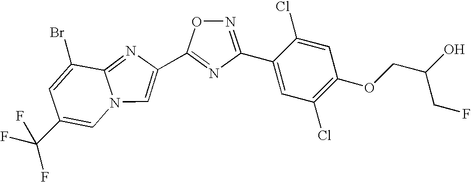 | 1-[(4-{5-[8-bromo-6- (trifluoromethyl)imidazo[1,2-a]pyridin-2-yl]- 1,2,4-oxadiazol-3-yl}-2,5-dichlorophenyl)oxy]-3- fluoropropan-2-ol |
| 111 | 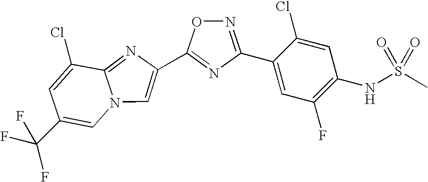 | N-(5-chloro-4-{5-[8-chloro-6- (trifluoromethyl)imidazo[1,2-a]pyridin-2-yl]- 1,2,4-oxadiazol-3-yl}-2- fluorophenyl)methanesulfonamide |
| 112 | 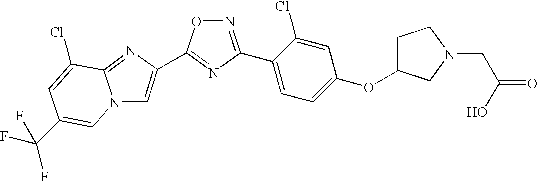 | {3-[(3-chloro-4-5-[chloro-6- trifluoromethyl)imidazo[1,2-a]pyridin-2-yl]- 1,2,4-oxadiazol-3-yl}phenyl)oxy]pyrrolidin-1- yl}acetic acid |
| 113 | 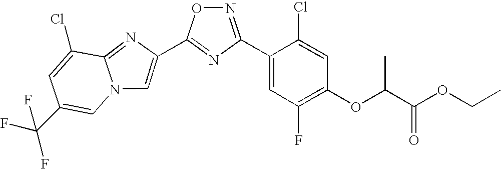 | ethyl 2-[(5-chloro-4-{5-[8-chloro-6- (trifluoromethyl)imidazo[1,2-a]pyridin-2-yl]- 1,2,4-oxadiazol-3-yl}-2- fluorophenyl)oxy]propanoate |
| 114 | 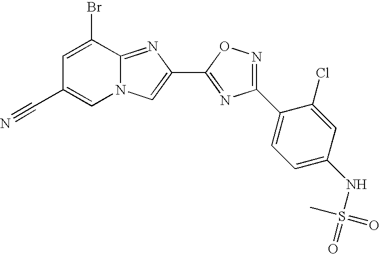 | N-{4-[5-(8-bromo-6-cyanoimidazo[1,2-a]pyridin- 2-yl)-1,2,4-oxadiazol-3-yl]-3- chlorophenyl}methanesulfonamide |
| 115 | 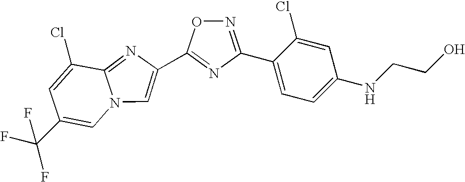 | 2-[(3-chloro-4-{5-[8-chloro-6- (trifluoromethyl)imidazo[1,2-a]pyridin-2-yl]- 1,2,4-oxadiazol-3-yl}phenyl)amino]ethanol |
| 116 | 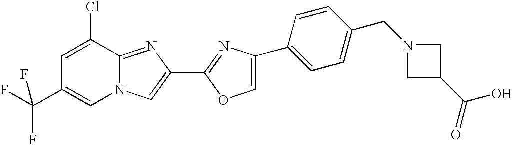 | 1-[(4-{5-[8-chloro-6- (trifluoromethyl)imidazo[1,2-a]pyridin-2-yl]- 1,2,4-oxadiazol-3-yl}phenyl)methyl]azetidine-3- carboxylic acid |
| 117 | 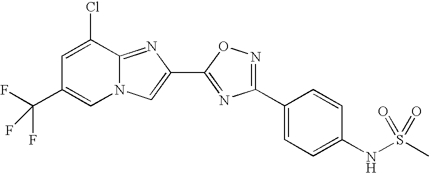 | N-(4-{5-[8-chloro-6- (trifluoromethyl)imidazo[1,2-a]pyridin-2-yl]- 1,2,4-oxadiazol-3-yl}phenyl)methanesulfonamide |
| 118 | 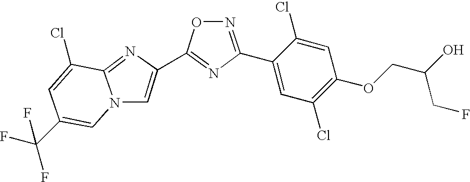 | 1-[(2,5-dichloro-4-{5-[8-chloro-6- (trifluoromethyl)imidazo[1,2-a]pyridin-2-yl]- 1,2,4-oxadiazol-3-yl}phenyl)oxy]-3-fluoropropan- 2-ol |
| 119 | 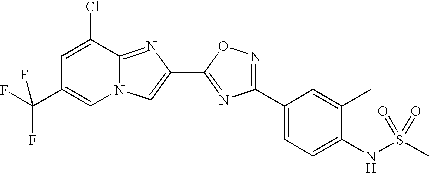 | N-(4-{5-[8-chloro-6- (trifluoromethyl)imidazo[1,2-a]pyridin-2-yl]- 1,2,4-oxadiazol-3-yl}-2- methylphenyl)methanesulfonamide |
| 120 | 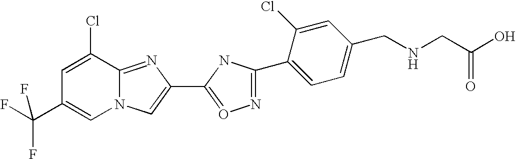 | N-[(3-chloro-4-{5-[8-chloro-6- (trifluoromethyl)imidazo[1,2-a]pyridin-2-yl]- 1,2,4-oxadiazol-3-yl}phenyl)methyl]glycine |
| 121 | 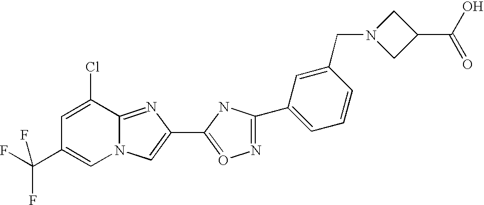 | 1-[(3-{5-[8-chloro-6- (trifluoromethyl)imidazo[1,2-a]pyridin-2-yl]- 1,2,4-oxadiazol-3-yl}phenyl)methyl]azetidine-3- carboxylic acid |
| 122 | 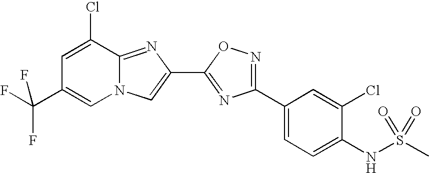 | N-(2-chloro-4-{5-[8-chloro-6- (trifluoromethyl)imidazo[1,2-a]pyridin-2-yl]- 1,2,4-oxadiazol-3-yl}phenyl)methanesulfonamide |
| 123 | 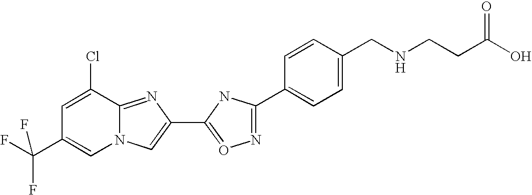 | N-[(4-{5-[8-chloro-6- (trifluoromethyl)imidazo[1,2-a]pyridin-2-yl]- 1,2,4-oxadiazol-3-yl}phenyl)methyl]-beta-alanine |
| 124 | 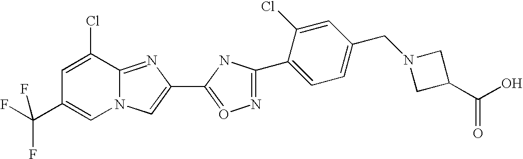 | 1-[(3-chloro-4-{5-[8-chloro-6- (trifluoromethyl)imidazo[1,2-a]pyridin-2-yl]- 1,2,4-oxadiazol-3-yl}phenyl)methyl]azetidine-3- carboxylic acid |
| 125 | 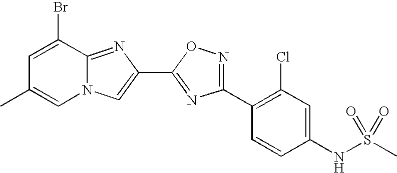 | N-{4-[5-(8-bromo-6-methylimidazo[1,2- a]pyridin-2-yl)-1,2,4-oxadiazol-3-yl]-3- chlorophenyl}methanesulfonamide |
| 126 | 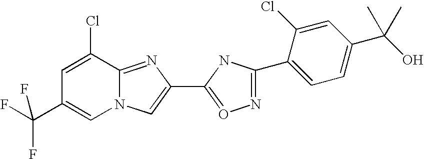 | 2-(3-chloro-4-{5-[8-chloro-6- (trifluoromethyl)imidazo[1,2-a]pyridin-2-yl]- 1,2,4-oxadiazol-3-yl}phenyl)propan-2-ol |
| 127 | 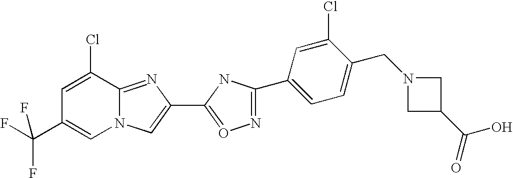 | 1-[(2-chloro-4-{5-[8-chloro-6- (trifluoromethyl)imidazo[1,2-a]pyridin-2-yl]- 1,2,4-oxadiazol-3-yl}phenyl)methyl]azetidine-3- carboxylic acid |
| 128 | 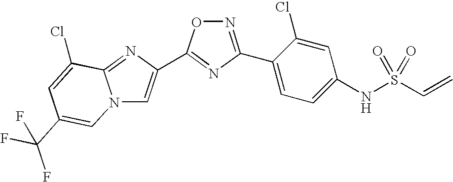 | N-(3-chloro-4-{5-[8-chloro-6- (trifluoromethyl)imidazo[1,2-a]pyridin-2-yl]- 1,2,4-oxadiazol-3-yl}phenyl)ethenesulfonamide |
| 129 | 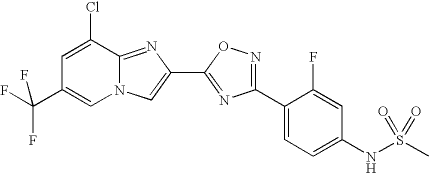 | N-(4-{5-[8-chloro-6- (trifluoromethyl)imidazo[1,2-a]pyridin-2-yl]- 1,2,4-oxadiazol-3-yl}-3- fluorophenyl)methanesulfonamide |
| 130 | 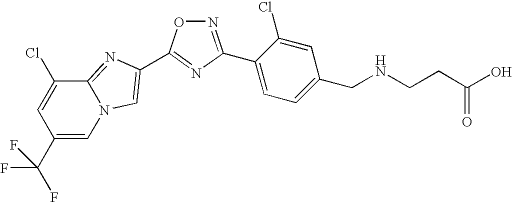 | N-[(3-chloro-4-{5-[8-chloro-6- (trifluoromethyl)imidazo[1,2-a]pyridin-2-yl]- 1,2,4-oxadiazol-3-yl}phenyl)methyl]-beta-alanine |
| 131 | 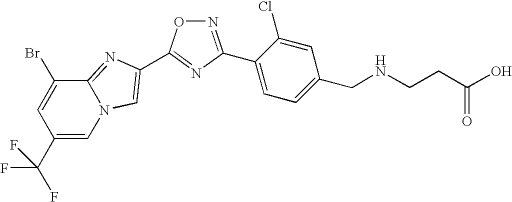 | N-[(4-{5-[8-bromo-6- (trifluoromethyl)imidazo[1,2-a]pyridin-2-yl]- 1,2,4-oxadiazol-3-yl}-3-chlorophenyl)methyl]- beta-alanine |
| 132 | 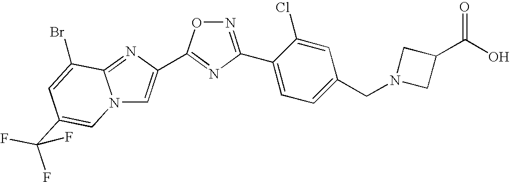 | 1-[(4-{5-[8-bromo-6- (trifluoromethyl)imidazo[1,2-a]pyridin-2-yl]- 1,2,4-oxadiazol-3-yl}-3- chlorophenyl)methyl]azetidine-3-carboxylic acid |
| 133 | 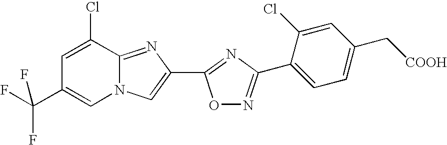 | (3-chloro-4-{5-[8-chloro-6- (trifluoromethyl)imidazo[1,2-a]pyridin-2-yl]- 1,2,4-oxadiazol-3-yl}phenyl)acetic acid |
| 134 | 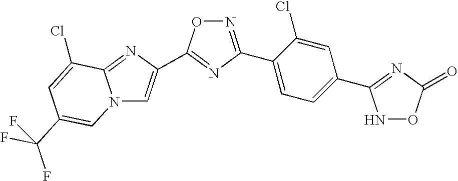 | 3-(3-chloro-4-{5-[8-chloro-6- (trifluoromethyl)imidazo[1,2-a]pyridin-2-yl]- 1,2,4-oxadiazol-3-yl}phenyl)-1,2,4-oxadiazol- 5(2H)-one |
| 135 | 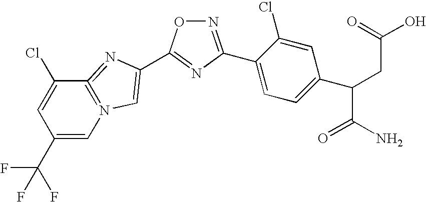 | 4-amino-3-(3-chloro-4-{5-[8-chloro-6- (trifluoromethyl)imidazo[1,2-a]pyridin-2-yl]- 1,2,4-oxadiazol-3-yl}phenyl)-4-oxobutanoic acid |
| 136 | 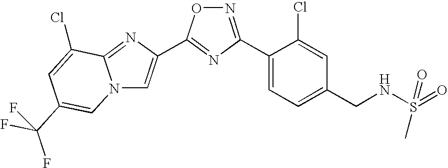 | N-[(3-chloro-4-{5-[8-chloro-6- (trifluoromethyl)imidazo[1,2-a]pyridin-2-yl]- 1,2,4-oxadiazol-3- yl}phenyl)methyl]methanesulfonamide |
| 137 | 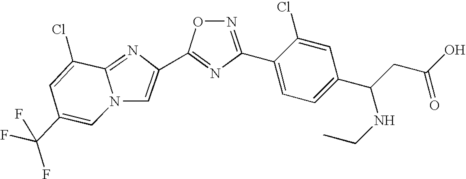 | 3-(3-chloro-4-{5-[8-chloro-6- (trifluoromethyl)imidazo[1,2-a]pyridin-2-yl]- 1,2,4-oxadiazol-3-yl}phenyl)-3- (ethylamino)propanoic acid |
| 138 | 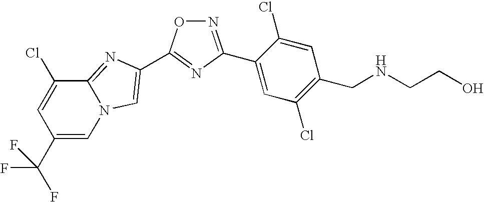 | 2-{[(2,5-dichloro-4-{5-[8-chloro-6- (trifluoromethyl)imidazo[1,2-a]pyridin-2-yl]- 1,2,4-oxadiazol-3- yl}phenyl)methyl]amino}ethanol |
| 139 | 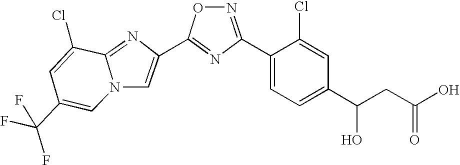 | 3-(3-chloro-4-{5-[8-chloro-6- (trifluoromethyl)imidazo[1,2-a]pyridin-2-yl]- 1,2,4-oxadiazol-3-yl}phenyl)-3-hydroxypropanoic acid |
| 140 | 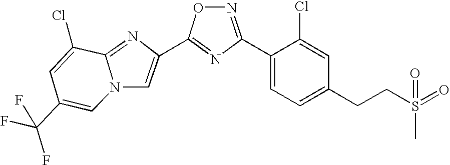 | 8-chloro-2-(3-{2-chloro-4-[2- (methylsulfonyl)ethyl]phenyl}-1,2,4-oxadiazol-5- yl)-6-(trifluoromethyl)imidazo[1,2-a]pyridine |
| 141 | 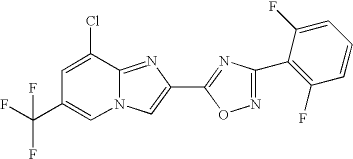 | 8-chloro-2-[3-(2,6-difluorophenyl)-1,2,4- oxadiazol-5-yl]-6-(trifluoromethyl)imidazo[1,2- a]pyridine |
| 142 | 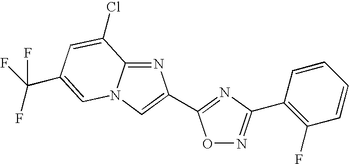 | 8-chloro-2-[3-(2-fluorophenyl)-1,2,4-oxadiazol-5- yl]-6-(trifluoromethyl)imidazo[1,2-a]pyridine |
| 143 | 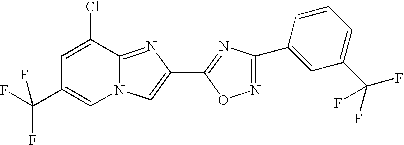 | 8-chloro-6-(trifluoromethyl)-2-{3-[3- (trifluoromethyl)phenyl]-1,2,4-oxadiazol-5- yl}imidazo[1,2-a]pyridine |
| 144 | 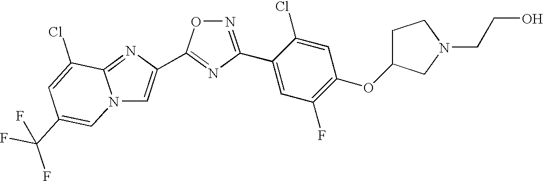 | 2-{3-[(5-chloro-4-{5-[8-chloro-6- (trifluoromethyl)imidazo[1,2-a]pyridin-2-yl]- 1,2,4-oxadiazol-3-yl}-2- fluorophenyl)oxy]pyrrolidin-1-yl}ethanol |
| 145 | 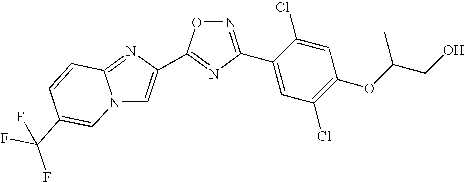 | 2-[(2,5-dichloro-4-{5-[8-chloro-6- (trifluoromethyl)imidazo[1,2-a]pyridin-2-yl]- 1,2,4-oxadiazol-3-yl}phenyl)oxy]propan-1-ol |
| 146 | 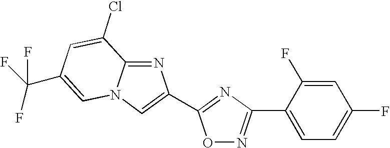 | 8-chloro-2-[3-(2,4-difluorophenyl)-1,2,4- oxadiazol-5-yl]-6-(trifluoromethyl)imidazo[1,2- a]pyridine |
| 147 | 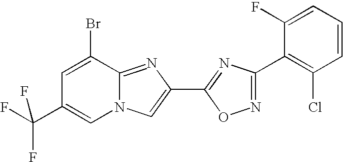 | 8-bromo-2-[3-(2-chloro-6-fluorophenyl)-1,2,4- oxadiazol-5-yl]-6-(trifluoromethyl)imidazo[1,2- a]pyridine |
| 148 | 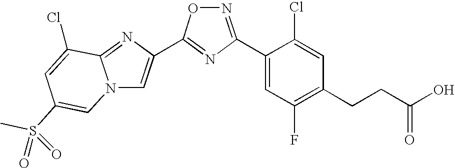 | 3-(5-chloro-4-{5-[8-chloro-6- (methylsulfonyl)imidazo[1,2-a]pyridin-2-yl]- 1,2,4-oxadiazol-3-yl}-2-fluorophenyl)propanoic acid |
| 149 | 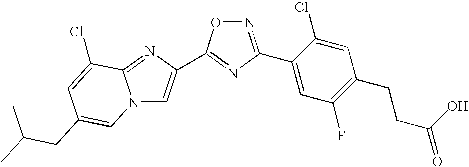 | 3-(5-chloro-4-{5-[8-chloro-6-(2- methylpropyl)imidazo[1,2-a]pyridin-2-yl]-1,2,4- oxadiazol-3-yl}-2-fluorophenyl)propanoic acid |
| 150 | 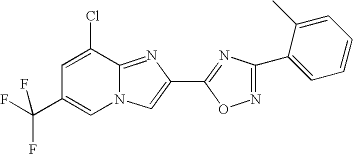 | 8-chloro-2-[3-(2-methylphenyl)-1,2,4-oxadiazol- 5-yl]-6-(trifluoromethyl)imidazo[1,2-a]pyridine |
| 151 | 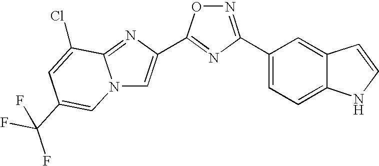 | 8-chloro-2-[3-(1H-indol-5-yl)-1,2,4-oxadiazol-5- yl]-6-(trifluoromethyl)imidazo[1,2-a]pyridine |
| 152 | 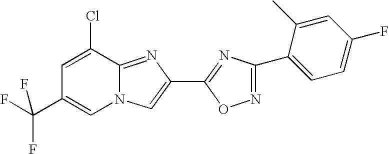 | 8-chloro-2-[3-(4-fluoro-2-methylphenyl)-1,2,4- oxadiazol-5-yl]-6-(trifluoromethyl)imidazo[1,2- a]pyridine |
| 153 | 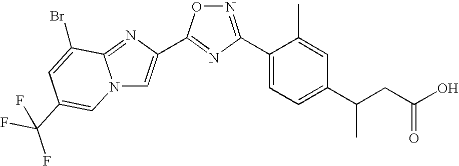 | 3-(4-{5-[8-bromo-6-(trifluoromethyl)imidazo[1,2- a]pyridin-2-yl]-1,2,4-oxadiazol-3-yl}-3- methylphenyl)butanoic acid |
| 154 | 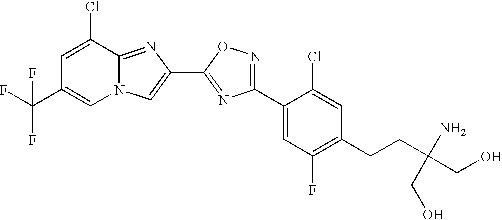 | 2-amino-2-[2-(5-chloro-4-{5-[8-chloro-6- (trifluoromethyl)imidazo[1,2-a]pyridin-2-yl]- 1,2,4-oxadiazol-3-yl}-2- fluorophenyl)ethyl]propane-1,3-diol |
| 155 | 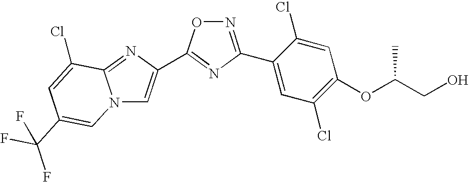 | (2R)-2-[(2,5-dichloro-4-{5-[8-chloro-6- (trifluoromethyl)imidazo[1,2-a]pyridin-2-yl]- 1,2,4-oxadiazol-3-yl}phenyl)oxy]propan-1-ol |
| 156 | 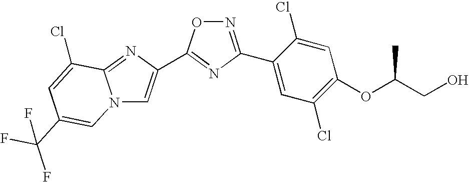 | (2S)-2-[(2,5-dichloro-4-{5-[8-chloro-6- (trifluoromethyl)imidazo[1,2-a]pyridin-2-yl]- 1,2,4-oxadiazol-3-yl}phenyl)oxy]propan-1-ol |
| 157 | 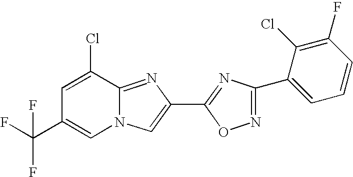 | 8-chloro-2-[3-(2-chloro-3-fluorophenyl)-1,2,4- oxadiazol-5-yl]-6-(trifluoromethyl)imidazo[1,2- a]pyridine |
| 158 | 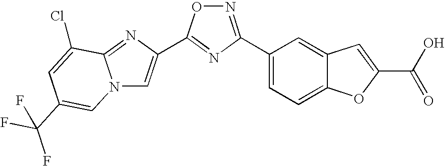 | 5-{5-[8-chloro-6-(trifluoromethyl)imidazo[1,2- a]pyridin-2-yl]-1,2,4-oxadiazol-3-yl}-1- benzofuran-2-carboxylic acid |
| 159 | 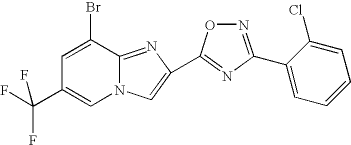 | 8-bromo-2-[3-(2-chlorophenyl)-1,2,4-oxadiazol-5- yl]-6-(trifluoromethyl)imidazo[1,2-a]pyridine |
| 160 | 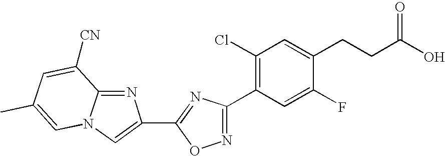 | 3-{5-chloro-4-[5-(8-cyano-6-methylimidazo[1,2- a]pyridin-2-yl)-1,2,4-oxadiazol-3-yl]-2- fluorophenyl}propanoic acid |
| 161 | 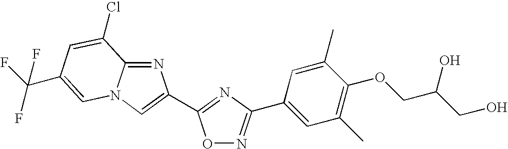 | 3-[(4-{5-[8-chloro-6- (trifluoromethyl)imidazo[1,2-a]pyridin-2-yl]- 1,2,4-oxadiazol-3-yl}-2,6- dimethylphenyl)oxy]propane-1,2-diol |
| 162 | 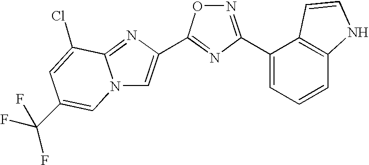 | 8-chloro-2-[3-(1H-indol-4-yl)-1,2,4-oxadiazol-5- yl]-6-(trifluoromethyl)imidazo[1,2-a]pyridine |
| 163 | 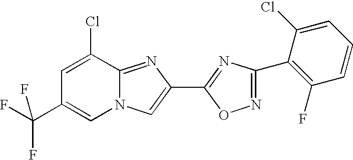 | 8-chloro-2-[3-(2-chloro-6-fluorophenyl)-1,2,4- oxadiazol-5-yl]-6-(trifluoromethyl)imidazo[1,2- a]pyridine |
| 164 | 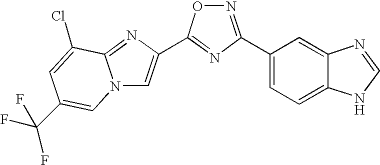 | 5-{5-[8-chloro-6-(trifluoromethyl)imidazo[1,2- a]pyridin-2-yl]-1,2,4-oxadiazol-3-yl}-1H- benzimidazole |
| 165 | 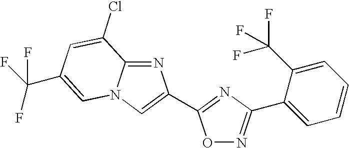 | 8-chloro-6-(trifluoromethyl)-2-{3-[2- (trifluoromethyl)phenyl]-1,2,4-oxadiazol-5- yl}imidazo[1,2-a]pyridine |
| 166 | 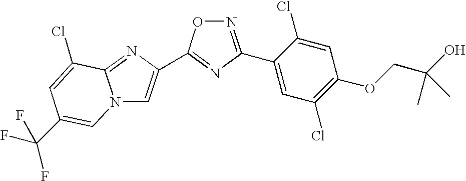 | 1-[(2,5-dichloro-4-{5-[8-chloro-6- (trifluoromethyl)imidazo[1,2-a]pyridin-2-yl]- 1,2,4-oxadiazol-3-yl}phenyl)oxy]-2- methylpropan-2-ol |
| 167 | 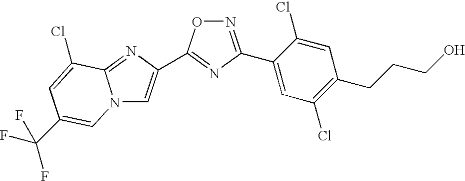 | 3-(2,5-dichloro-4-{5-[8-chloro-6- (trifluoromethyl)imidazo[1,2-a]pyridin-2-yl]- 1,2,4-oxadiazol-3-yl}phenyl)propan-1-ol |
| 168 | 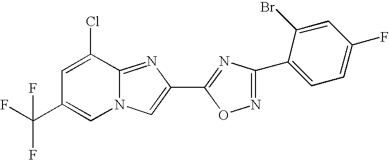 | 2-[3-(2-bromo-4-fluorophenyl)-1,2,4-oxadiazol-5- yl]-8-chloro-6-(trifluoromethyl)imidazo[1,2- a]pyridine |
| 169 | 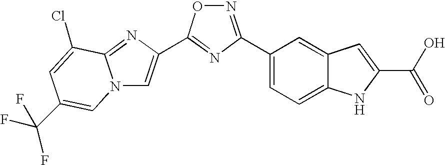 | 5-{5-[8-chloro-6-(trifluoromethyl)imidazo[1,2- a]pyridin-2-yl]-1,2,4-oxadiazol-3-yl}-1H-indole- 2-carboxylic acid |
| 170 | 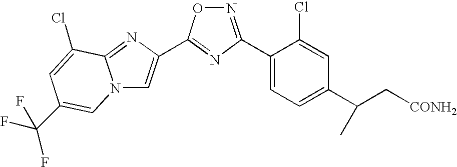 | 3-(3-chloro-4-{5-[8-chloro-6- (trifluoromethyl)imidazo[1,2-a]pyridin-2-yl]- 1,2,4-oxadiazol-3-yl}phenyl)butanamide |
| 171 | 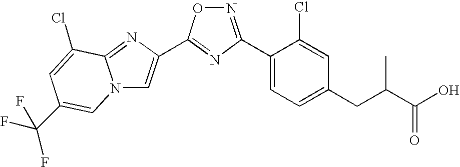 | 3-(3-chloro-4-{5-[8-chloro-6- (trifluoromethyl)imidazo[1,2-a]pyridin-2-yl]- 1,2,4-oxadiazol-3-yl}phenyl)-2-methylpropanoic acid |
| 172 | 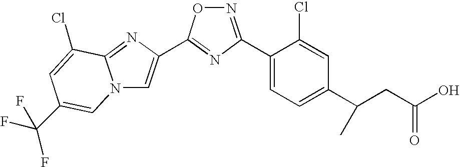 | 3-(3-chloro-4-{5-[8-chloro-6- (trifluoromethyl)imidazo[1,2-a]pyridin-2-yl]- 1,2,4-oxadiazol-3-yl}phenyl)butanoic acid |
| 173 | 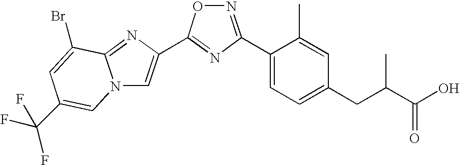 | 3-(4-{5-[8-bromo-6-(trifluoromethyl)imidazo[1,2- a]pyridin-2-yl]-1,2,4-oxadiazol-3-yl}-3- methylphenyl)-2-methylpropanoic acid |
| 174 | 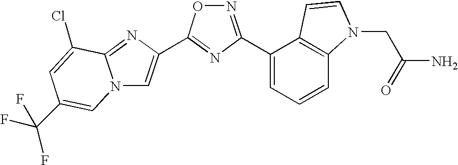 | 2-(4-{5-[8-chloro-6-(trifluoromethyl)imidazo[1,2- a]pyridin-2-yl]-1,2,4-oxadiazol-3-yl}-1H-indol-1- yl)acetamide |
| 175 | 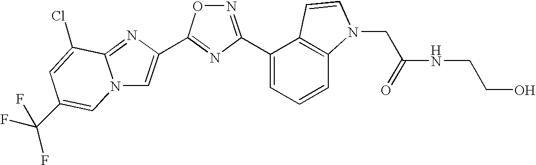 | 2-(4-{5-[8-chloro-6-(trifluoromethyl)imidazo[1,2- a]pyridin-2-yl]-1,2,4-oxadiazol-3-yl}-1H-indol-1- yl)-N-(2-hydroxyethyl)acetamide |
| 176 | 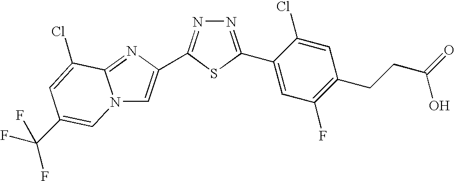 | 3-(5-chloro-4-{5-[8-chloro-6- (trifluoromethyl)imidazo[1,2-a]pyridin-2-yl]- 1,3,4-thiadiazol-2-yl}-2-fluorophenyl)propanoic acid |
| 177 | 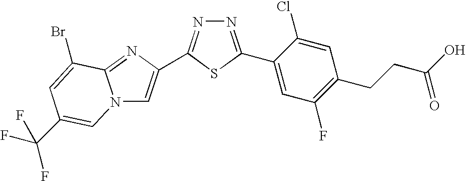 | 3-(4-{5-[8-bromo-6-(trifluoromethyl)imidazo[1,2- a]pyridin-2-yl]-1,3,4-thiadiazol-2-yl}-5-chloro-2- fluorophenyl)propanoic acid |
| 178 | 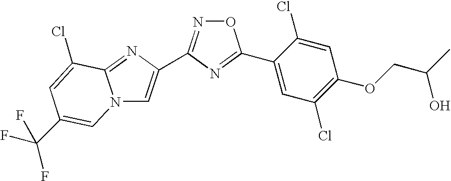 | 1-[(2,5-dichloro-4-{3-[8-chloro-6- (trifluoromethyl)imidazo[1,2-a]pyridin-2-yl]- 1,2,4-oxadiazol-5-yl}phenyl)oxy]propan-2-ol |
| 179 | 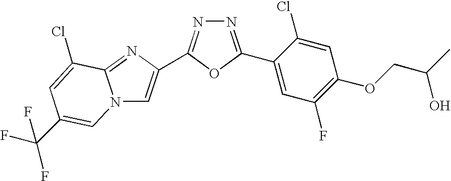 | 1-[(5-chloro-4-{5-[8-chloro-6- (trifluoromethyl)imidazo[1,2-a]pyridin-2-yl]- 1,3,4-oxadiazol-2-yl}-2-fluorophenyl)oxy]propan- 2-ol |
| 180 | 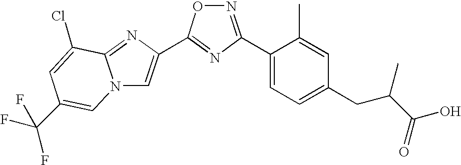 | 3-(4-{5-[8-chloro-6-(trifluoromethyl)imidazo[1,2- a]pyridin-2-yl]-1,2,4-oxadiazol-3-yl}-3- methylphenyl)-2-methylpropanoic acid |
| 181 | 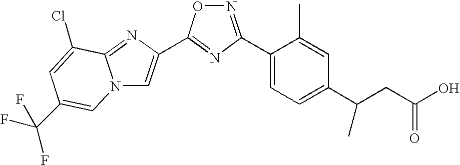 | 3-(4-{5-[8-chloro-6-(trifluoromethyl)imidazo[1,2- a]pyridin-2-yl]-1,2,4-oxadiazol-3-yl}-3- methylphenyl)butanoic acid |
| 182 | 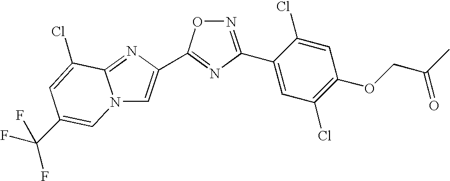 | 1-[(2,5-dichloro-4-{5-[8-chloro-6- trifluoromethyl)imidazo[1,2-a]pyridin-2-yl]- 1,2,4-oxadiazol-3-yl}phenyl)oxy]propan-2-one |
| 183 | 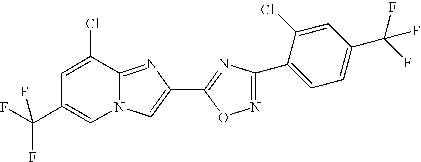 | 8-chloro-2-{3-[2-chloro-4- (trifluoromethyl)phenyl]-1,2,4-oxadiazol-5-yl}-6- (trifluoromethyl)imidazo[1,2-a]pyridine |
| 184 | 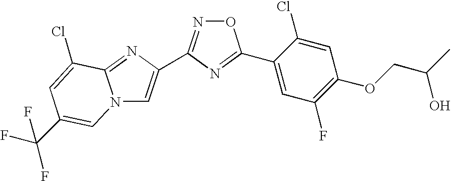 | 1-[(5-chloro-4-{3-[8-chloro-6- (trifluoromethyl)imidazo[1,2-a]pyridin-2-yl]- 1,2,4-oxadiazol-5-yl}-2-fluorophenyl)oxy]propan- 2-ol |
| 185 | 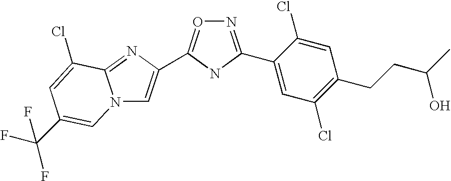 | 4-(2,5-dichloro-4-{5-[8-chloro-6- (trifluoromethyl)imidazo[1,2-a]pyridin-2-yl]- 1,2,4-oxadiazol-3-yl}phenyl)butan-2-ol |
| 186 | 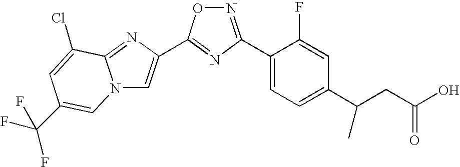 | 3-(4-{5-[8-chloro-6-(trifluoromethyl)imidazo[1,2- a]pyridin-2-yl]-1,2,4-oxadiazol-3-yl}-3- fluorophenyl)butanoic acid |
| 187 | 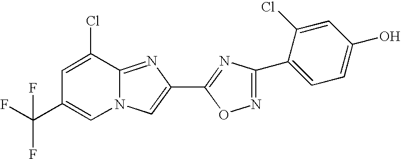 | 3-chloro-4-{5-[8-chloro-6- (trifluoromethyl)imidazo[1,2-a]pyridin-2-yl]- 1,2,4-oxadiazol-3-yl}phenol |
| 188 | 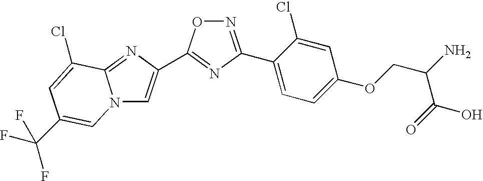 | O-(3-chloro-4-{5-[8-chloro-6- (trifluoromethyl)imidazo[1,2-a]pyridin-2-yl]- 1,2,4-oxadiazol-3-yl}phenyl)serine |
| 189 | 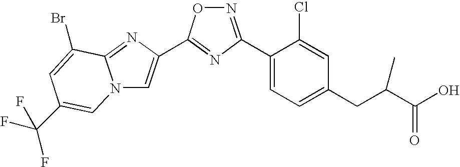 | 3-(4-{5-[8-bromo-6-(trifluoromethyl)imidazo[1,2- a]pyridin-2-yl]-1,2,4-oxadiazol-3-yl}-3- chlorophenyl)-2-methylpropanoic acid |
| 190 | 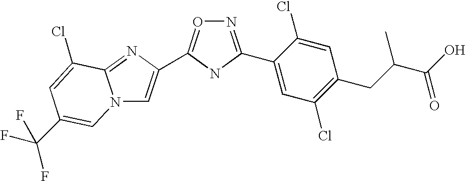 | 3-(2,5-dichloro-4-{5-[8-chloro-6- (trifluoromethyl)imidazo[1,2-a]pyridin-2-yl]- 1,2,4-oxadiazol-3-yl}phenyl)-2-methylpropanoic acid |
| 191 | 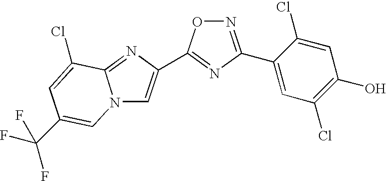 | 2,5-dichloro-4-{5-[8-chloro-6- (trifluoromethyl)imidazo[1,2-a]pyridin-2-yl]- 1,2,4-oxadiazol-3-yl}phenol |
| 192 | 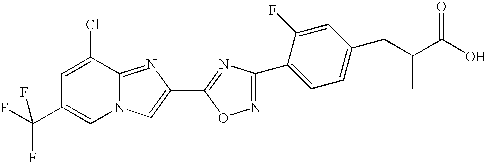 | 3-(4-{5-[8-chloro-6-(trifluoromethyl)imidazo[1,2- a]pyridin-2-yl]-1,2,4-oxadiazol-3-yl}-3- fluorophenyl)-2-methylpropanoic acid |
| 193 | 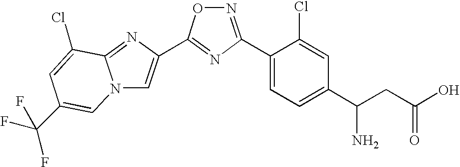 | 3-amino-3-(3-chloro-4-{5-[8-chloro-6- (trifluoromethyl)imidazo[1,2-a]pyridin-2-yl]- 1,2,4-oxadiazol-3-yl}phenyl)propanoic acid |
| 194 | 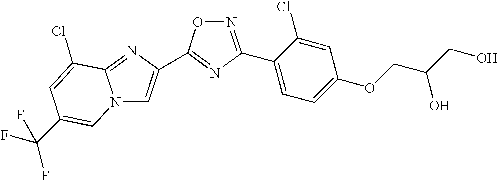 | 3-[(3-chloro-4-{5-[8-chloro-6- (trifluoromethyl)imidazo[1,2-a]pyridin-2-yl]- 1,2,4-oxadiazol-3-yl}phenyl)oxy]propane-1,2-diol |
| 195 | 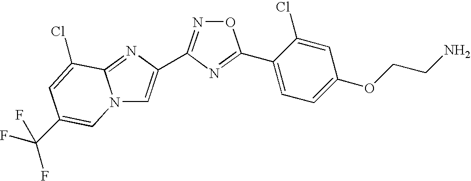 | 2-[(3-chloro-4-{3-[8-chloro-6- (trifluoromethyl)imidazo[1,2-a]pyridin-2-yl]- 1,2,4-oxadiazol-5-yl}phenyl)oxy]ethanamine |
| 196 | 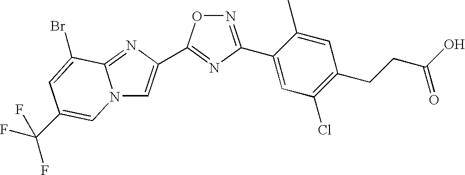 | 3-(4-{5-[8-bromo-6-(trifluoromethyl)imidazo[1,2- a]pyridin-2-yl]-1,2,4-oxadiazol-3-yl}-2-chloro-5- methylphenyl)propanoic acid |
| 197 | 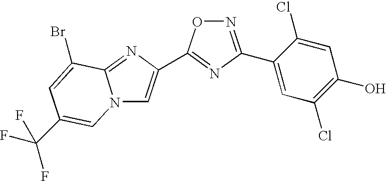 | 4-{5-[8-bromo-6-(trifluoromethyl)imidazo[1,2- a]pyridin-2-yl]-1,2,4-oxadiazol-3-yl}-2,5- dichlorophenol |
| 198 | 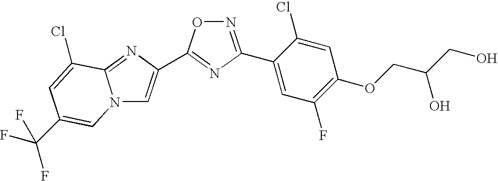 | 3-[(5-chloro-4-{5-[8-chloro-6- (trifluoromethyl)imidazo[1,2-a]pyridin-2-yl]- 1,2,4-oxadiazol-3-yl}-2- fluorophenyl)oxy]propane-1,2-diol |
| 199 | 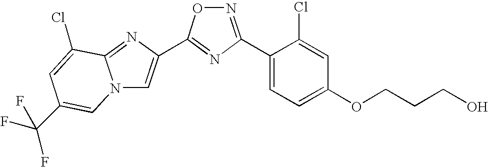 | 3-[(3-chloro-4-{5-[8-chloro-6- (trifluoromethyl)imidazo[1,2-a]pyridin-2-yl]- 1,2,4-oxadiazol-3-yl}phenyl)oxy]propan-1-ol |
| 200 | 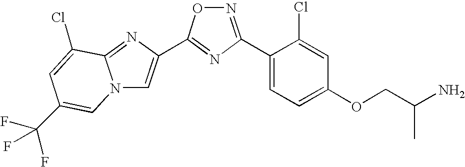 | 1-[(3-chloro-4-{5-[8-chloro-6- (trifluoromethyl)imidazo[1,2-a]pyridin-2-yl]- 1,2,4-oxadiazol-3-yl}phenyl)oxy]propan-2-amine |
| 201 | 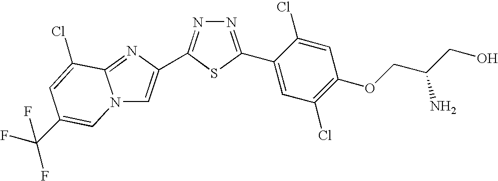 | (2S)-2-amino-3-[(2,5-dichloro-4-{5-[8-chloro-6- (trifluoromethyl)imidazo[1,2-a]pyridin-2-yl]- 1,3,4-thiadiazol-2-yl}phenyl)oxy]propan-1-ol |
| 202 | 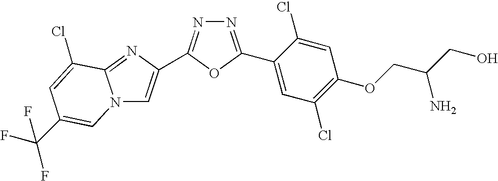 | 2-amino-3-[(2,5-dichloro-4-{5-[8-chloro-6- (trifluoromethyl)imidazo[1,2-a]pyridin-2-yl]- 1,3,4-oxadiazol-2-yl}phenyl)oxy]propan-1-ol |
| 203 | 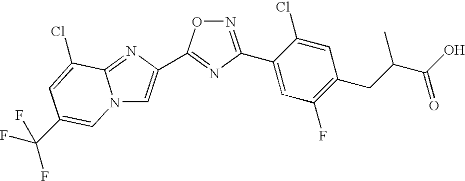 | 3-(5-chloro-4-{5-[8-chloro-6- (trifluoromethyl)imidazo[1,2-a]pyridin-2-yl]- 1,2,4-oxadiazol-3-yl}-2-fluorophenyl)-2- methylpropanoic acid |
| 204 | 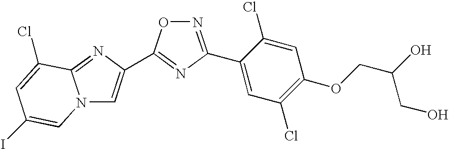 | 3-({2,5-dichloro-4-[5-(8-chloro-6- iodoimidazo[1,2-a]pyridin-2-yl)-1,2,4-oxadiazol- 3-yl]phenyl}oxy)propane-1,2-diol |
| 205 | 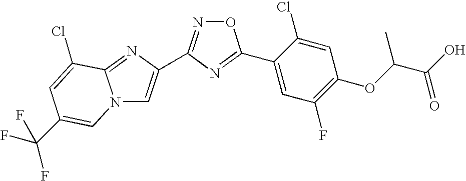 | 2-[(5-chloro-4-{3-[8-chloro-6- (trifluoromethyl)imidazo[1,2-a]pyridin-2-yl]- 1,2,4-oxadiazol-5-yl}-2- fluorophenyl)oxy]propanoic acid |
| 206 | 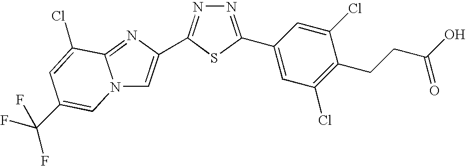 | 3-(2,6-dichloro-4-{5-[8-chloro-6- (trifluoromethyl)imidazo[1,2-a]pyridin-2-yl]- 1,3,4-thiadiazol-2-yl}phenyl)propanoic acid |
| 207 | 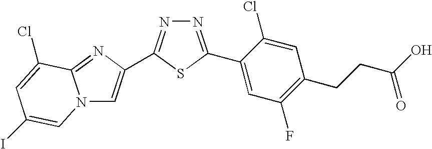 | 3-{5-chloro-4-[5-(8-chloro-6-iodoimidazo[1,2- a]pyridin-2-yl)-1,3,4-thiadiazol-2-yl]-2- fluorophenyl}propanoic acid |
| 208 | 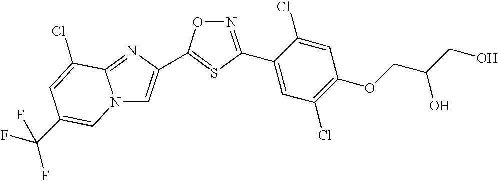 | 3-[(2,5-dichloro-4-{5-[8-chloro-6- (trifluoromethyl)imidazo[1,2-a]pyridin-2-yl]- 1,2,4-oxadiazol-3-yl}phenyl)oxy]propane-1,2-diol |
| 209 | 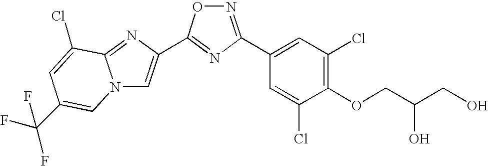 | 3-[(2,6-dichloro-4-{5-[8-chloro-6- (trifluoromethyl)imidazo[1,2-a]pyridin-2-yl]- 1,2,4-oxadiazol-3-yl}phenyl)oxy]propane-1,2-diol |
| 210 | 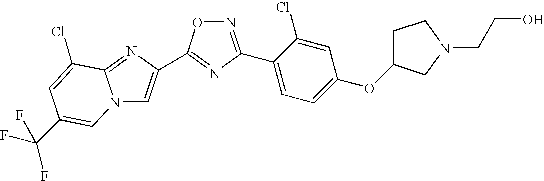 | 2-{3-[(3-chloro-4-{5-[8-chloro-6- (trifluoromethyl)imidazo[1,2-a]pyridin-2-yl]- 1,2,4-oxadiazol-3-yl}phenyl)oxy]pyrrolidin-1- yl}ethanol |
| 211 | 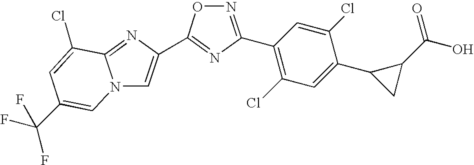 | 2-(2,5-dichloro-4-{5-[8-chloro-6- (trifluoromethyl)imidazo[1,2-a]pyridin-2-yl]- 1,2,4-oxadiazol-3- yl}phenyl)cyclopropanecarboxylic acid |
| 212 | 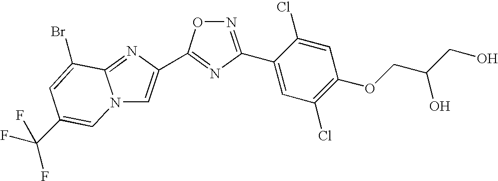 | 3-[(4-{5-[8-bromo-6- (trifluoromethyl)imidazo[1,2-a]pyridin-2-yl]- 1,2,4-oxadiazol-3-yl}-2,5- dichlorophenyl)oxy]propane-1,2-diol |
| 213 | 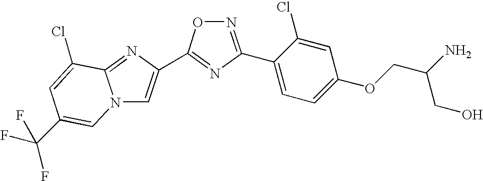 | 2-amino-3-[(3-chloro-4-{5-[8-chloro-6- (trifluoromethyl)imidazo[1,2-a]pyridin-2-yl]- 1,2,4-oxadiazol-3-yl}phenyl)oxy]propan-1-ol |
| 214 | 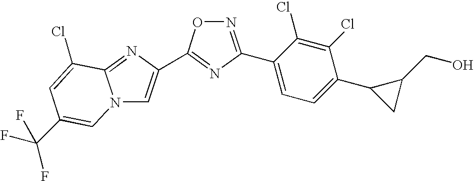 | [2-(3-chloro-4-{5-[8-chloro-6- (trifluoromethyl)imidazo[1,2-a]pyridin-2-yl]- 1,2,4-oxadiazol-3- yl}phenyl)cyclopropyl]methanol |
| 215 | 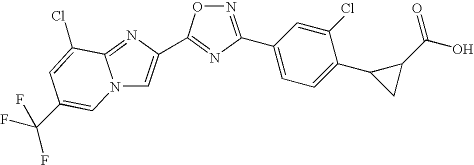 | 2-(2-chloro-4-{5-[8-chloro-6- (trifluoromethyl)imidazo[1,2-a]pyridin-2-yl]- 1,2,4-oxadiazol-3- yl}phenyl)cyclopropanecarboxylic acid |
| 216 | 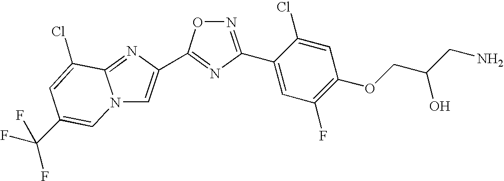 | 1-amino-3-[(5-chloro-4-{5-[8-chloro-6- (trifluoromethyl)imidazo[1,2-a]pyridin-2-yl]- 1,2,4-oxadiazol-3-yl}-2-fluorophenyl)oxy]propan- 2-ol |
| 217 | 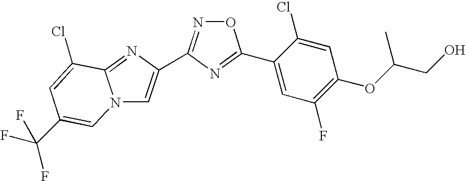 | 2-[(5-chloro-4-{3-[8-chloro-6- (trifluoromethyl)imidazo[1,2-a]pyridin-2-yl]- 1,2,4-oxadiazol-5-yl}-2-fluorophenyl)oxy]propan- 1-ol |
| 218 | 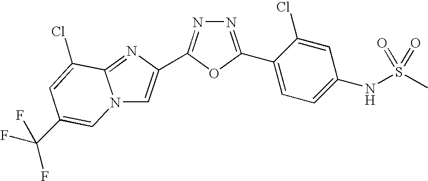 | N-(3-chloro-4-{5-[8-chloro-6- (trifluoromethyl)imidazo[1,2-a]pyridin-2-yl]- 1,3,4-oxadiazol-2-yl}phenyl)methanesulfonamide |
| 219 | 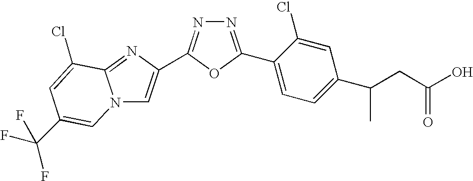 | 3-(3-chloro-4-{5-[8-chloro-6- (trifluoromethyl)imidazo[1,2-a]pyridin-2-yl]- 1,3,4-oxadiazol-2-yl}phenyl)butanoic acid |
| 220 | 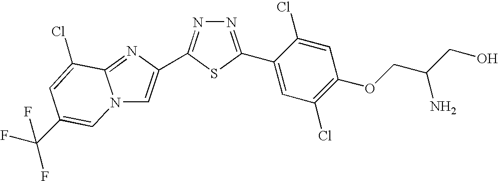 | 2-amino-3-[(2,5-dichloro-4-{5-[8-chloro-6- (trifluoromethyl)imidazo[1,2-a]pyridin-2-yl]- 1,3,4-thiadiazol-2-yl}phenyl)oxy]propan-1-ol |
| 221 | 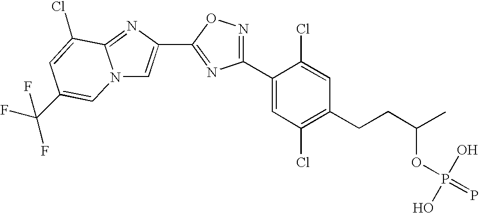 | 3-(2,5-dichloro-4-{5-[8-chloro-6- (trifluoromethyl)imidazo[1,2-a]pyridin-2-yl]- 1,2,4-oxadiazol-3-yl}phenyl)-1-methylpropyl dihydrogen phosphate |
| 222 | 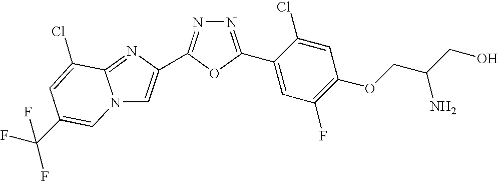 | 2-amino-3-[(5-chloro-4-{5-[8-chloro-6- (trifluoromethyl)imidazo[1,2-a]pyridin-2-yl]- 1,3,4-oxadiazol-2-yl}-2-fluorophenyl)oxy]propan- 1-ol |
| 223 | 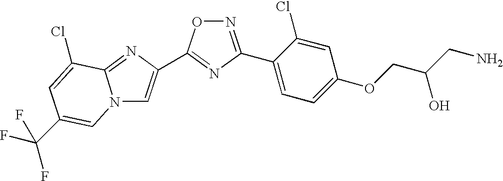 | 1-amino-3-[(3-chloro-4-{5-[8-chloro-6- (trifluoromethyl)imidazo[1,2-a]pyridin-2-yl]- 1,2,4-oxadiazol-3-yl}phenyl)oxy]propan-2-ol |
| 224 | 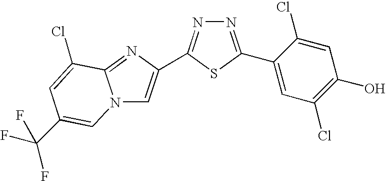 | 2,5-dichloro-4-{5-[8-chloro-6- (trifluoromethyl)imidazo[1,2-a]pyridin-2-yl]- 1,3,4-thiadiazol-2-yl}phenol |
| 225 | 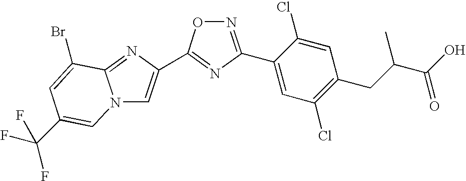 | 3-(4-{5-[8-bromo-6-(trifluoromethyl)imidazo[1,2- a]pyridin-2-yl]-1,2,4-oxadiazol-3-yl}-2,5- dichlorophenyl)-2-methylpropanoic acid |
| 226 | 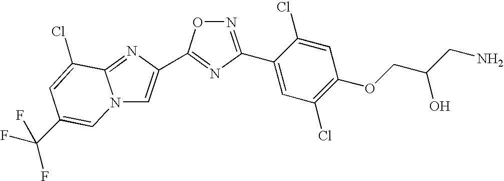 | 1-amino-3-[(2,5-dichloro-4-{5-[8-chloro-6- (trifluoromethyl)imidazo[1,2-a]pyridin-2-yl]- 1,2,4-oxadiazol-3-yl}phenyl)oxy]propan-2-ol |
| 227 | 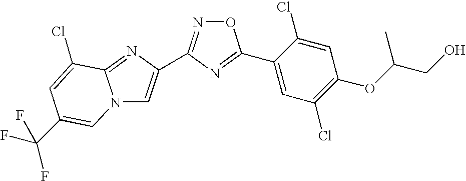 | 2-[(2,5-dichloro-4-{3-[8-chloro-6- (trifluoromethyl)imidazo[1,2-a]pyridin-2-yl]- 1,2,4-oxadiazol-5-yl}phenyl)oxy]propan-1-ol |
| 228 | 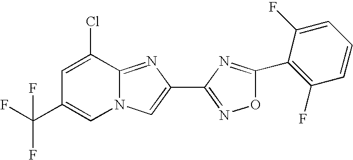 | 8-chloro-2-[5-(2,6-difluorophenyl)-1,2,4- oxadiazol-3-yl]-6-(trifluoromethyl)imidazo[1,2- a]pyridine |
| 229 | 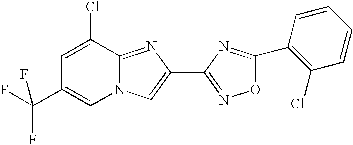 | 8-chloro-2-[5-(2-chlorophenyl)-1,2,4-oxadiazol-3- yl]-6-(trifluoromethyl)imidazo[1,2-a]pyridine |
| 230 | 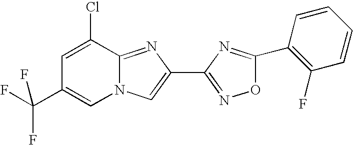 | 8-chloro-2-[5-(2-fluorophenyl)-1,2,4-oxadiazol-3- yl]-6-(trifluoromethyl)imidazo[1,2-a]pyridine |
| 231 | 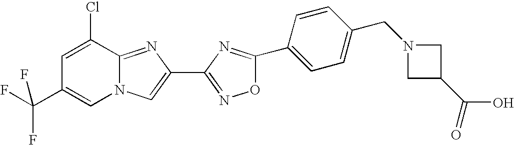 | 1-[(4-{3-[8-chloro-6- (trifluoromethyl)imidazo[1,2-a]pyridin-2-yl]- 1,2,4-oxadiazol-5-yl}phenyl)methyl]azetidine-3- carboxylic acid |
| 232 | 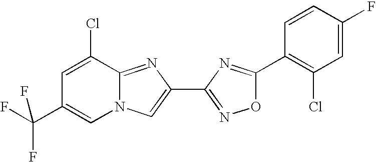 | 8-chloro-2-[5-(2-chloro-4-fluorophenyl)-1,2,4- oxadiazol-3-yl]-6-(trifluoromethyl)imidazo[1,2- a]pyridine |
| 233 | 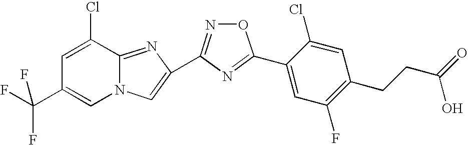 | 3-(5-chloro-4-{3-[8-chloro-6- (trifluoromethyl)imidazo[1,2-a]pyridin-2-yl]- 1,2,4-oxadiazol-5-yl}-2-fluorophenyl)propanoic acid |
| 234 | 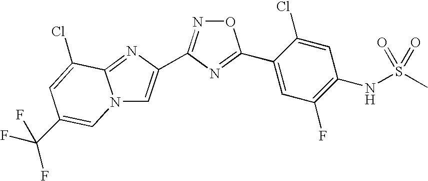 | N-(5-chloro-4-{3-[8-chloro-6- (trifluoromethyl)imidazo[1,2-a]pyridin-2-yl]- 1,2,4-oxadiazol-5-yl}-2- fluorophenyl)methanesulfonamide |
| 235 | 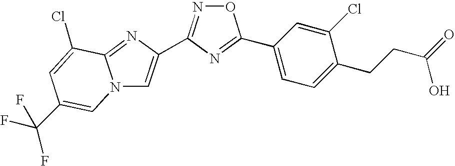 | 3-(2-chloro-4-{3-[8-chloro-6- (trifluoromethyl)imidazo[1,2-a]pyridin-2-yl]- 1,2,4-oxadiazol-5-yl}phenyl)propanoic acid |
| 236 | 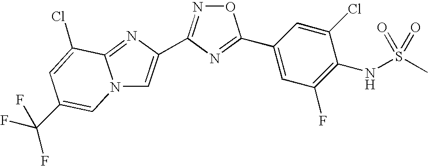 | N-(2-chloro-4-{3-[8-chloro-6- (trifluoromethyl)imidazo[1,2-a]pyridin-2-yl]- 1,2,4-oxadiazol-5-yl}-6- fluorophenyl)methane sulfonamide |
| 237 | 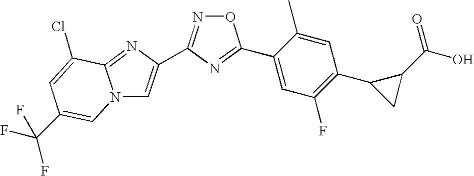 | 2-(4-{3-[8-chloro-6-(trifluoromethyl)imidazo[1,2- a]pyridin-2-yl]-1,2,4-oxadiazol-5-yl}-2-fluoro-5- methylphenyl)cyclopropanecarboxylic acid |
| 238 | 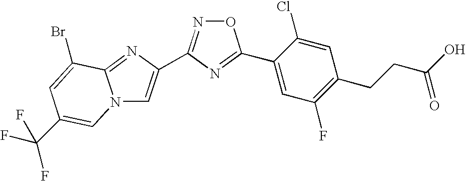 | 3-(4-{3-[8-bromo-6-(trifluoromethyl)imidazo[1,2- a]pyridin-2-yl]-1,2,4-oxadiazol-5-yl}-5-chloro-2- fluorophenyl)propanoic acid |
| 239 | 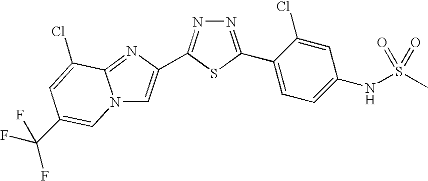 | N-(3-chloro-4-{5-[8-chloro-6- (trifluoromethyl)imidazo[1,2-a]pyridin-2-yl]- 1,3,4-thiadiazol-2-yl}phenyl)methanesulfonamide |
| 240 | 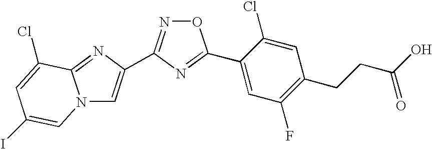 | 3-{5-chloro-4-[3-(8-chloro-6-iodoimidazo[1,2- a]pyridin-2-yl)-1,2,4-oxadiazol-5-yl]-2- fluorophenyl}propanoic acid |
| 241 | 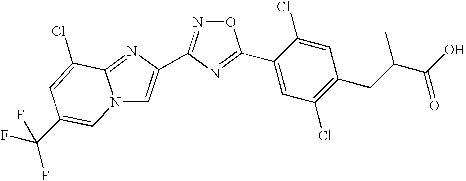 | 3-(2,5-dichloro-4-{3-[8-chloro-6- (trifluoromethyl)imidazo[1,2-a]pyridin-2-yl]- 1,2,4-oxadiazol-5-yl}phenyl)-2-methylpropanoic acid |
| 242 | 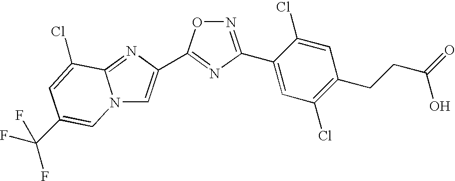 | 3-(2,5-dichloro-4-{5-[8-chloro-6- (trifluoromethyl)imidazo[1,2-a]pyridin-2-yl]- 1,2,4-oxadiazol-3-yl}phenyl)propanoic acid |
| 243 | 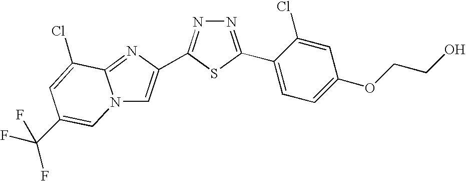 | 2-[(3-chloro-4-{5-[8-chloro-6- (trifluoromethyl)imidazo[1,2-a]pyridin-2-yl]- 1,3,4-thiadiazol-2-yl}phenyl)oxy]ethanol |
| 244 | 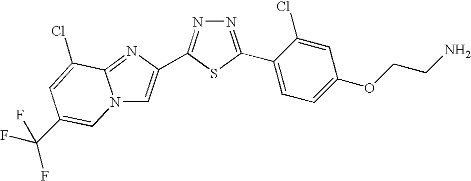 | 2-[(3-chloro-4-{5-[8-chloro-6- (trifluoromethyl)imidazo[1,2-a]pyridin-2-yl]- 1,3,4-thiadiazol-2-yl}phenyl)oxy]ethanamine |
| 245 | 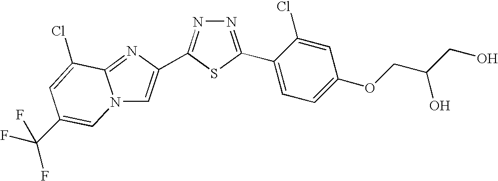 | 3-[(3-chloro-4-{5-[8-chloro-6- (trifluoromethyl)imidazo[1,2-a]pyridin-2-yl]- 1,3,4-thiadiazol-2-yl}phenyl)oxy]propane-1,2- diol |
| 246 | 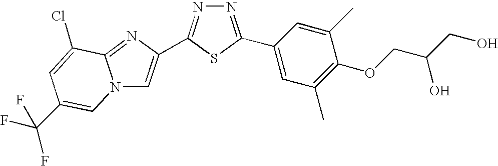 | 3-[(4-{5-[8-chloro-6- (trifluoromethyl)imidazo[1,2-a]pyridin-2-yl]- 1,3,4-thiadiazol-2-yl}-2,6- dimethylphenyl)oxy]propane-1,2-diol |
| 247 | 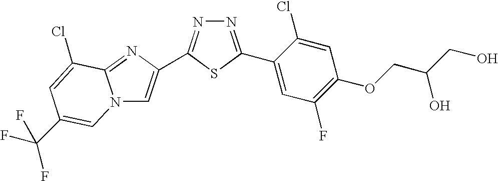 | 3-[(5-chloro-4-{5-[8-chloro-6- (trifluoromethyl)imidazo[1,2-a]pyridin-2-yl]- 1,3,4-thiadiazol-2-yl}-2- fluorophenyl)oxy]propane-1,2-diol |
| 248 | 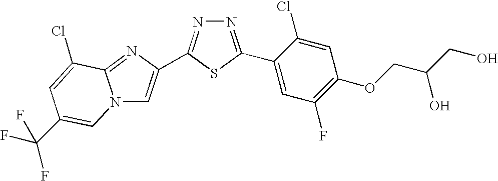 | 2-[(5-chloro-4-{5-[8-chloro-6- (trifluoromethyl)imidazo[1,2-a]pyridin-2-yl]- 1,3,4-thiadiazol-2-yl}-2- fluorophenyl)oxy]propan-1-ol |
| 249 | 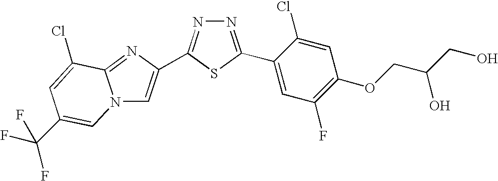 | 1-amino-3-[(5-chloro-4-{5-[8-chloro-6- (trifluoromethyl)imidazo[1,2-a]pyridin-2-yl]- 1,3,4-thiadiazol-2-yl}-2- fluorophenyl)oxy]propan-2-ol |
| 250 | 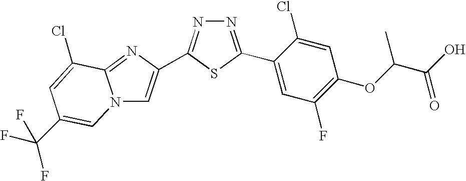 | 2-[(5-chloro-4-{5-[8-chloro-6- (trifluoromethyl)imidazo[1,2-a]pyridin-2-yl]- 1,3,4-thiadiazol-2-yl}-2- fluorophenyl)oxy]propanoic acid |
| 251 | 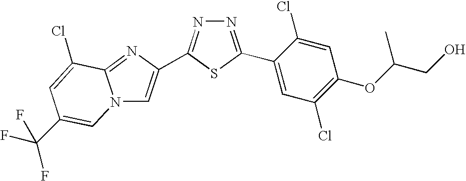 | 2-[(2,5-dichloro-4-{5-[8-chloro-6- (trifluoromethyl)imidazo[1,2-a]pyridin-2-yl]- 1,3,4-thiadiazol-2-yl}phenyl)oxy]propan-1-ol |
| 252 | 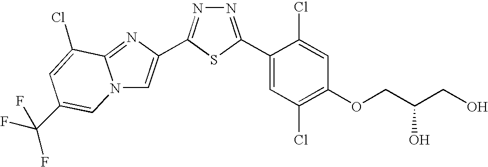 | (25)-3-[(2,5-dichloro-4-{5-[8-chloro-6- (trifluoromethyl)imidazo[1,2-a]pyridin-2-yl]- 1,3,4-thiadiazol-2-yl}phenyl)oxy]propane-1,2- diol |
| 253 | 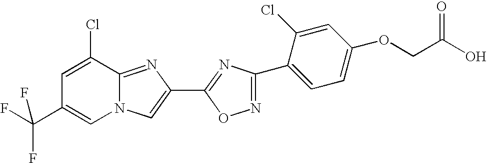 | [(3-chloro-4-{5-[8-chloro-6- (trifluoromethyl)imidazo[1,2-a]pyridin-2-yl]- 1,2,4-oxadiazol-3-yl}phenyl)oxy]acetic acid |
| 254 | 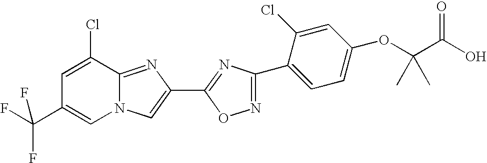 | 2-[(3-chloro-4-{5-[8-chloro-6- (trifluoromethyl)imidazo[1,2-a]pyridin-2-yl]- 1,2,4-oxadiazol-3-yl}phenyl)oxy]-2- methylpropanoic acid |
| 255 | 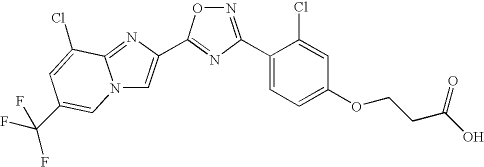 | 3-[(3-chloro-4-{5-[8-chloro-6- (trifluoromethyl)imidazo[1,2-a]pyridin-2-yl]- 1,2,4-oxadiazol-3-yl}phenyl)oxy]propanoic acid |
| 256 | 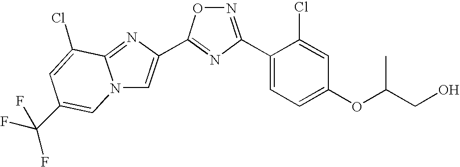 | 2-[(3-chloro-4-{5-[8-chloro-6- (trifluoromethyl)imidazo[1,2-a]pyridin-2-yl]- 1,2,4-oxadiazol-3-yl}phenyl)oxy]propan-1-ol |
| 257 | 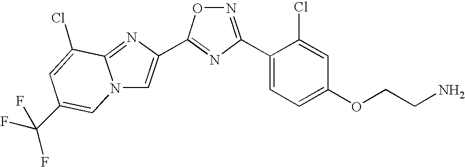 | 2-[(3-chloro-4-{5-[8-chloro-6- (trifluoromethyl)imidazo[1,2-a]pyridin-2-yl]- 1,2,4-oxadiazol-3-yl}phenyl)oxy]ethanamine |
| 258 | 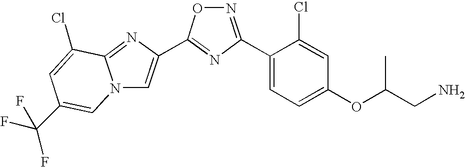 | 2-[(3-chloro-4-{5-[8-chloro-6- (trifluoromethyl)imidazo[1,2-a]pyridin-2-yl]- 1,2,4-oxadiazol-3-yl}phenyl)oxy]propan-1-amine |
| 259 | 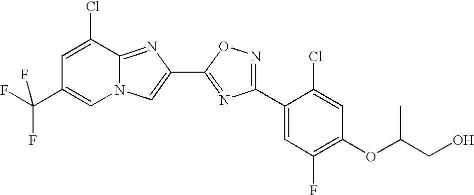 | 2-[(5-chloro-4-{5-[8-chloro-6- (trifluoromethyl)imidazo[1,2-a]pyridin-2-yl]- 1,2,4-oxadiazol-3-yl}-2-fluorophenyl)oxy]propan- 1-ol |
| 260 | 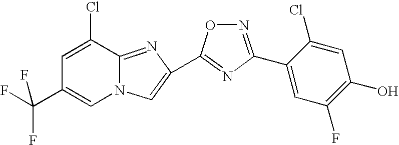 | 5-chloro-4-{5-[8-chloro-6- (trifluoromethyl)imidazo[1,2-a]pyridin-2-yl]- 1,2,4-oxadiazol-3-yl}-2-fluorophenol |
| 261 | 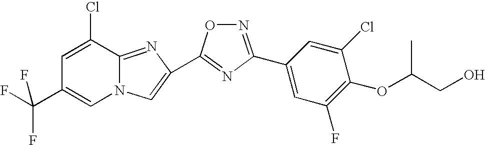 | 2-[(2-chloro-4-{5-[8-chloro-6- (trifluoromethyl)imidazo[1,2-a]pyridin-2-yl]- 1,2,4-oxadiazol-3-yl}-6-fluorophenyl)oxy]propan- 1-ol |
| 262 | 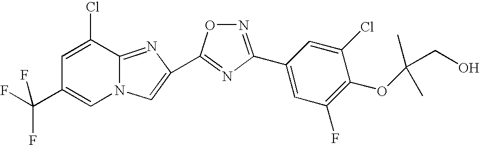 | 2-[(2-chloro-4-{5-[8-chloro-6- (trifluoromethyl)imidazo[1,2-a]pyridin-2-yl]- 1,2,4-oxadiazol-3-yl}-6-fluorophenyl)oxy]-2- methylpropan-1-ol |
| 263 | 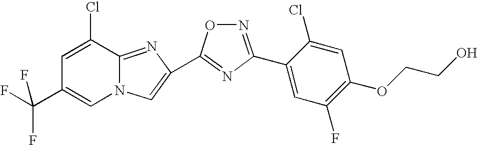 | 2-[(5-chloro-4-{5-[8-chloro-6- (trifluoromethyl)imidazo[1,2-a]pyridin-2-yl]- 1,2,4-oxadiazol-3-yl}-2-fluorophenyl)oxy]ethanol |
| 264 | 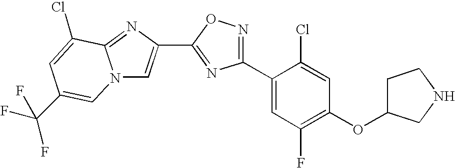 | 8-chloro-2-{3-[2-chloro-5-fluoro-4-(pyrrolidin-3- yloxy)phenyl]-1,2,4-oxadiazol-5-yl}-6- (trifluoromethyl)imidazo[1,2-a]pyridine |
| 265 | 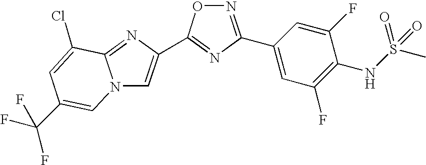 | N-(4-{5-[8-chloro-6- (trifluoromethyl)imidazo[1,2-a]pyridin-2-yl]- 1,2,4-oxadiazol-3-yl}-2,6- difluorophenyl)methanesulfonamide |
| 266 | 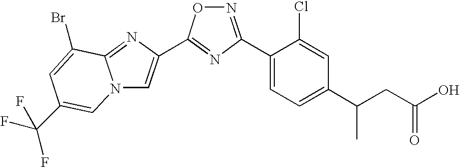 | 3-(4-{5-[8-bromo-6-(trifluoromethyl)imidazo[1,2- a]pyridin-2-yl]-1,2,4-oxadiazol-3-yl}-3- chlorophenyl)butanoic acid |
| 267 | 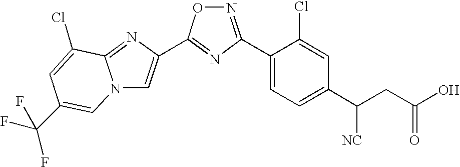 | 3-(3-chloro-4-{5-[8-chloro-6- (trifluoromethyl)imidazo[1,2-a]pyridin-2-yl]- 1,2,4-oxadiazol-3-yl}phenyl)-3-cyanopropanoic acid |
| 268 | 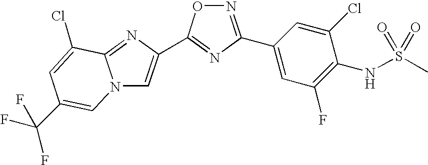 | N-(2-chloro-4-{5-[8-chloro-6- (trifluoromethyl)imidazo[1,2-a]pyridin-2-yl]- 1,2,4-oxadiazol-3-yl}-6- fluorophenyl)methane sulfonamide |
| 269 | 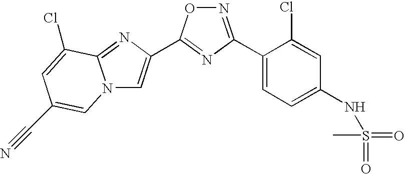 | N-{3-chloro-4-[5-(8-chloro-6-cyanoimidazo[1,2- a]pyridin-2-yl)-1,2,4-oxadiazol-3- yl]phenyl}methanesulfonamide |
| 270 | 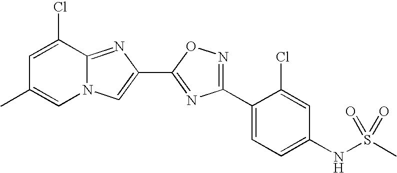 | N-{3-chloro-4-[5-(8-chloro-6-methylimidazo[1,2- a]pyridin-2-yl)-1,2,4-oxadiazol-3- yl]phenyl}methanesulfonamide |
| 271 | 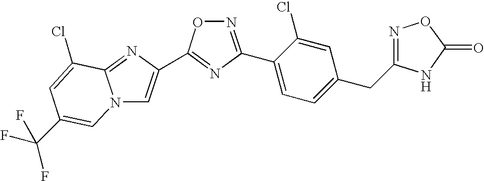 | 3-[(3-chloro-4-{5-[8-chloro-6- (trifluoromethyl)imidazo[1,2-a]pyridin-2-yl]- 1,2,4-oxadiazol-3-yl}phenyl)methyl]-1,2,4- oxadiazol-5(4H)-one |
| 272 | 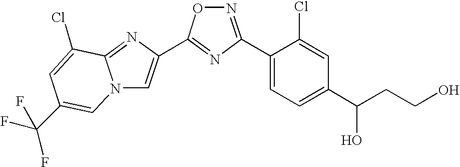 | 1-(3-chloro-4-{5-[8-chloro-6- (trifluoromethyl)imidazo[1,2-a]pyridin-2-yl]- 1,2,4-oxadiazol-3-yl}phenyl)propane-1,3-diol |
| 273 | 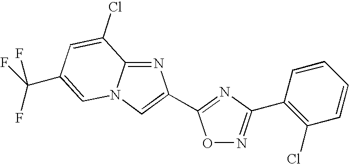 | 8-chloro-2-[3-(2-chlorophenyl)-1,2,4-oxadiazol-5- yl]-6-(trifluoromethyl)imidazo[1,2-a]pyridine |
| 274 | 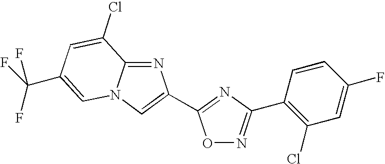 | 8-chloro-2-[3-(2-chloro-4-fluorophenyl)-1,2,4- oxadiazol-5-yl]-6-(trifluoromethyl)imidazo[1,2- a]pyridine |
| 275 | 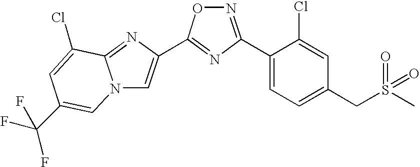 | 8-chloro-2-(3-{2-chloro-4- [(methylsulfonyl)methyl]phenyl}-1,2,4-oxadiazol- 5-yl)-6-(trifluoromethyl)imidazo[1,2-a]pyridine |
| 276 | 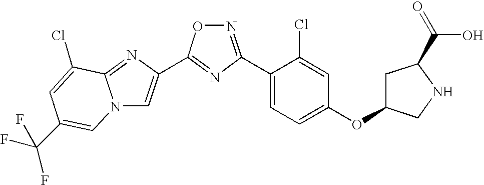 | (4S)-4-[(3-chloro-4-{5-[8-chloro-6- (trifluoromethyl)imidazo[1,2-a]pyridin-2-yl]- 1,2,4-oxadiazol-3-yl}phenyl)oxy]-L-proline |
| 277 | 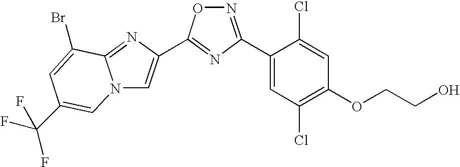 | 2-[(4-{5-[8-bromo-6- (trifluoromethyl)imidazo[1,2-a]pyridin-2-yl]- 1,2,4-oxadiazol-3-yl}-2,5- dichlorophenyl)oxy]ethanol |
| 278 | 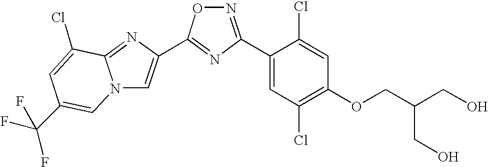 | 2-{[(2,5-dichloro-4-{5-[8-chloro-6- (trifluoromethyl)imidazo[1,2-a]pyridin-2-yl]- 1,2,4-oxadiazol-3- yl}phenyl)oxy]methyl}propane-1,3-diol |
| 279 | 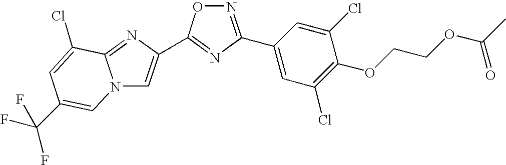 | 2-[(2,6-dichloro-4-{5-[8-chloro-6- (trifluoromethyl)imidazo[1,2-a]pyridin-2-yl]- 1,2,4-oxadiazol-3-yl}phenyl)oxy]ethyl acetate |
| 280 | 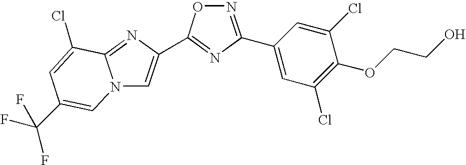 | 2-[(2,6-dichloro-4-{5-[8-chloro-6- (trifluoromethyl)imidazo[1,2-a]pyridin-2-yl]- 1,2,4-oxadiazol-3-yl}phenyl)oxy]ethanol |
| 281 | 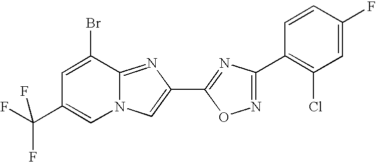 | 8-bromo-2-[3-(2-chloro-4-fluorophenyl)-1,2,4- oxadiazol-5-yl]-6-(trifluoromethyl)imidazo[1,2- a]pyridine |
| 282 | 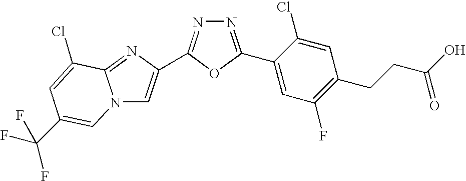 | 3-(5-chloro-4-{5-[8-chloro-6- (trifluoromethyl)imidazo[1,2-a]pyridin-2-yl]- 1,3,4-oxadiazol-2-yl}-2-fluorophenyl)propanoic acid |
| 283 | 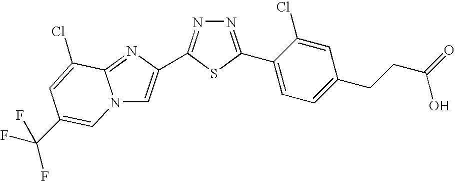 | 3-(3-chloro-4-{5-[8-chloro-6- (trifluoromethyl)imidazo[1,2-a]pyridin-2-yl]- 1,3,4-thiadiazol-2-yl}phenyl)propanoic acid |
| 284 | 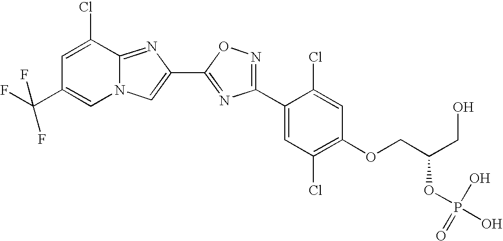 | (S)-1-(2,5-dichloro-4-(5-(8-chloro-6- (trifluoromethyl)imidazo[1,2-a]pyridin-2-yl)- 1,2,4-oxadiazol-3-yl)phenoxy)-3-hydroxypropan- 2-yl dihydrogen phosphate |
| 285 | 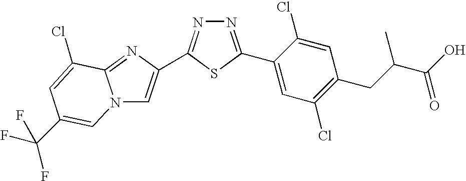 | 3-(2,5-dichloro-4-{5-[8-chloro-6- (trifluoromethyl)imidazo[1,2-a]pyridin-2-yl]- 1,3,4-thiadiazol-2-yl}phenyl)-2-methylpropanoic acid |
| 286 | 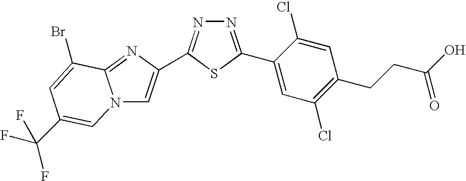 | 3-(4-{5-[8-bromo-6-(trifluoromethyl)imidazo[1,2- a]pyridin-2-yl]-1,3,4-thiadiazol-2-yl}-2,5- dichlorophenyl)propanoic acid |
| 287 | 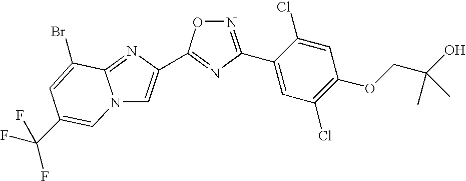 | 1-[(4-{5-[8-bromo-6- (trifluoromethyl)imidazo[1,2-a]pyridin-2-yl]- 1,2,4-oxadiazol-3-yl}-2,5-dichlorophenyl)oxy]-2- methylpropan-2-ol |
| 288 | 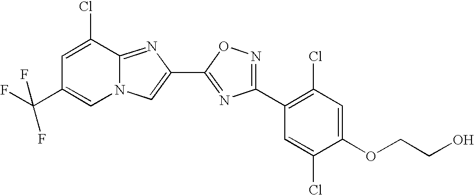 | 2-[(2,5-dichloro-4-{5-[8-chloro-6- (trifluoromethyl)imidazo[1,2-a]pyridin-2-yl]- 1,2,4-oxadiazol-3-yl}phenyl)oxy]ethanol |
| 289 | 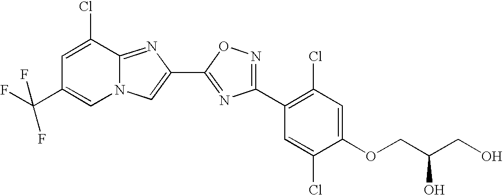 | (2R)-3-[(2,5-dichloro-4-{5-[8-chloro-6- (trifluoromethyl)imidazo[1,2-a]pyridin-2-yl]- 1,2,4-oxadiazol-3-yl}phenyl)oxy]propane-1,2-diol |
| 290 | 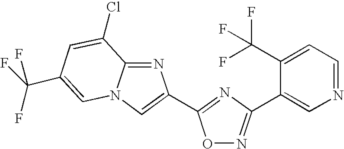 | 8-chloro-6-(trifluoromethyl)-2-{3-[4- (trifluoromethyl)pyridin-3-yl]-1,2,4-oxadiazol-5- yl}imidazo[1,2-a]pyridine |
| 291 | 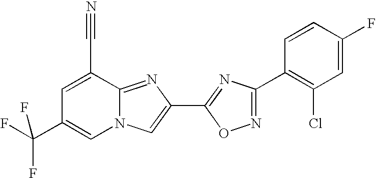 | 2-[3-(2-chloro-4-fluorophenyl)-1,2,4-oxadiazol-5- yl]-6-(trifluoromethyl)imidazo[1,2-a]pyridine-8- carbonitrile |
| 292 | 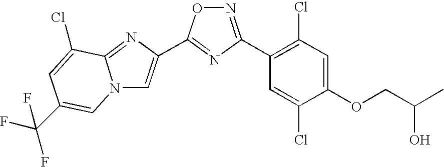 | 1-[(2,5-dichloro-4-{5-[8-chloro-6- (trifluoromethyl)imidazo[1,2-a]pyridin-2-yl]- 1,2,4-oxadiazol-3-yl}phenyl)oxy]propan-2-ol |
| 293 | 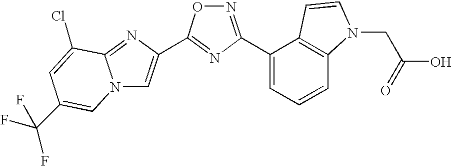 | (4-{5-[8-chloro-6-(trifluoromethyl)imidazo[1,2- a]pyridin-2-yl]-1,2,4-oxadiazol-3-yl}-1H-indol-1- yl)acetic acid |
| 294 | 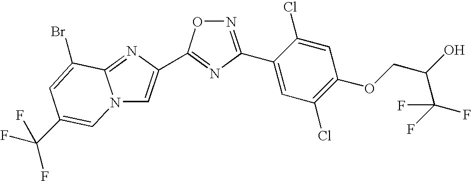 | 3-[(4-{5-[8-bromo-6- (trifluoromethyl)imidazo[1,2-a]pyridin-2-yl]- 1,2,4-oxadiazol-3-yl}-2,5-dichlorophenyl)oxy]- 1,1,1-trifluoropropan-2-ol |
| 295 | 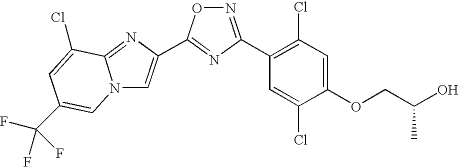 | (2R)-1-[(2,5-dichloro-4-{5-[8-chloro-6- (trifluoromethyl)imidazo[1,2-a]pyridin-2-yl]- 1,2,4-oxadiazol-3-yl}phenyl)oxy]propan-2-ol |
| 296 | 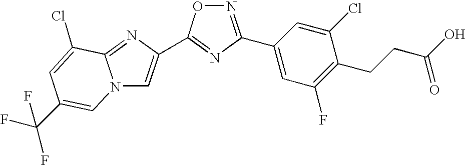 | 3-(2-chloro-4-{5-[8-chloro-6- (trifluoromethyl)imidazo[1,2-a]pyridin-2-yl]- 1,2,4-oxadiazol-3-yl}-6-fluorophenyl)propanoic acid |
| 297 | 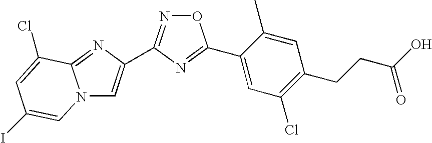 | 3-{2-chloro-4-[3-(8-chloro-6-iodoimidazo[1,2- a]pyridin-2-yl)-1,2,4-oxadiazol-5-yl]-5- methylphenyl}propanoic acid |
| 298 | 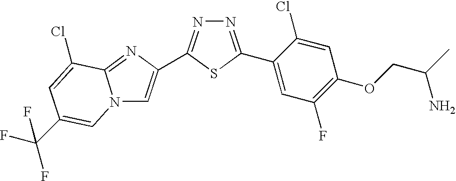 | 1-[(5-chloro-4-{5-[8-chloro-6- (trifluoromethyl)imidazo[1,2-a]pyridin-2-yl]- 1,3,4-thiadiazol-2-yl}-2- fluorophenyl)pxy]propan-2-amine |
| 299 | 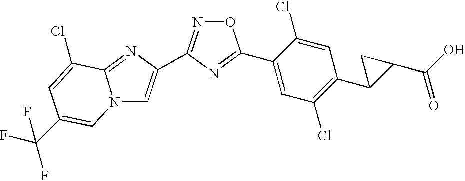 | 2-(2,5-dichloro-4-{3-[8-chloro-6- (trifluoromethyl)imidazo[1,2-a]pyridin-2-yl]- 1,2,4-oxadiazol-5- yl}phenyl)cyclopropanecarboxylic acid |
| 300 | 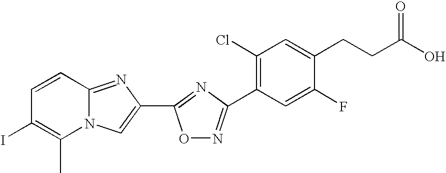 | 3-{5-chloro-2-fluoro-4-[5-(6-iodo-5- methylimidazo[1,2-a]pyridin-2-yl)-1,2,4- oxadiazol-3-yl]phenyl}propanoic acid |
| 301 | 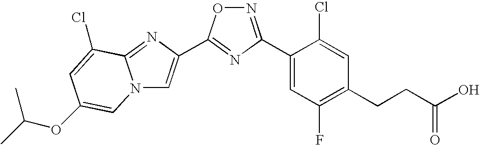 | 3-[5-chloro-4-(5-{8-chloro-6-[(1- methylethyl)oxy]imidazo[1,2-a]pyridin-2-yl}- 1,2,4-oxadiazol-3-yl)-2-fluorophenyl]propanoic acid |
| 302 | 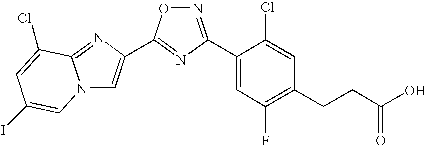 | 3-{5-chloro-4-[5-(8-chloro-6-iodoimidazo[1,2- a]pyridin-2-yl)-1,2,4-oxadiazol-3-yl]-2- fluorophenyl}propanoic acid |
| 303 | 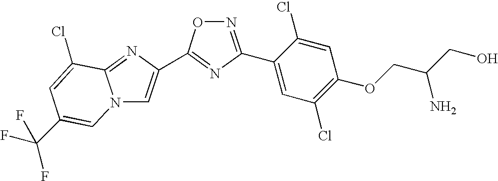 | 2-amino-3-[(2,5-dichloro-4-{5-[8-chloro-6- (trifluoromethyl)imidazo[1,2-a]pyridin-2-yl]- 1,2,4-oxadiazol-3-yl}phenyl)oxy]propan-1-ol |
| 304 | 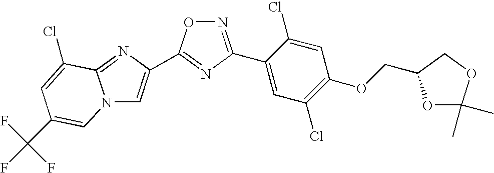 | 8-chloro-2-{3-[2,5-dichloro-4-({[(4R)-2,2- dimethyl-1,3-dioxolan-4-yl]methyl}oxy)phenyl]- 1,2,4-oxadiazol-5-yl}-6- (trifluoromethyl)imidazo[1,2-a]pyridine |
| 305 | 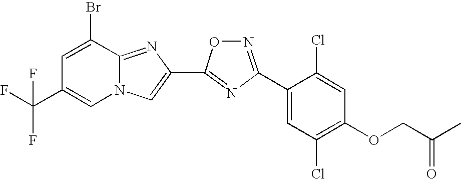 | 1-[(4-{5-[8-bromo-6- (trifluoromethyl)imidazo[1,2-a]pyridin-2-yl]- 1,2,4-oxadiazol-3-yl}-2,5- dichlorophenyl)oxy]propan-2-one |
| 306 | 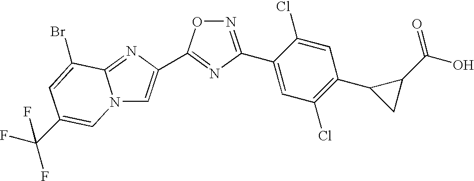 | 2-(4- {5-[8-bromo-6-(trifluoromethyl)imidazo[1,2- a]pyridin-2-yl]-1,2,4-oxadiazol-3-yl}-2,5- dichlorophenyl)cyclopropanecarboxylic acid |
| 307 | 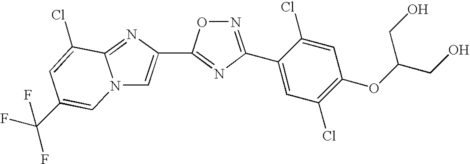 | 2-[(2,5-dichloro-4-{5-[8-chloro-6- (trifluoromethyl)imidazo[1,2-a]pyridin-2-yl]- 1,2,4-oxadiazol-3-yl}phenyl)oxy]propane-1,3-diol |
| 308 | 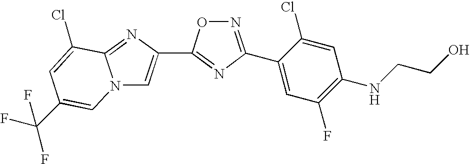 | 2-[(5-chloro-4-{5-[8-chloro-6- (trifluoromethyl)imidazo[1,2-a]pyridin-2-yl]- 1,2,4-oxadiazol-3-yl}-2- fluorophenyl)amino]ethanol |
| 309 | 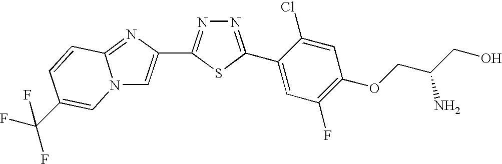 | (2S)-2-amino-3-[(5-chloro-2-fluoro-4-{5-[6- (trifluoromethyl)imidazo[1,2-a]pyridin-2-yl]- 1,3,4-thiadiazol-2-yl}phenyl)oxy]propan-1-ol |
| 310 | 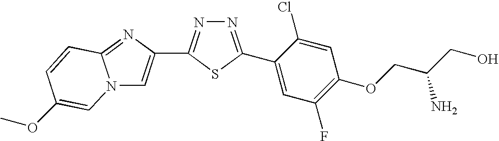 | (2S)-2-amino-3-[(5-chloro-2-fluoro-4-{5-[6- (methyloxy)imidazo[1,2-a]pyridin-2-yl]-1,3,4- thiadiazol-2-yl}phenyl)oxy]propan-1-ol |
| 311 | 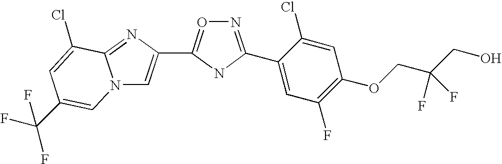 | 3-[(2,5-dichloro-4-{5-[8-chloro-6- (trifluoromethyl)imidazo[1,2-a]pyridin-2-yl]- 1,2,4-oxadiazol-3-yl}phenyl)oxy]-2,2- difluoropropan-1-ol |
| 312 | 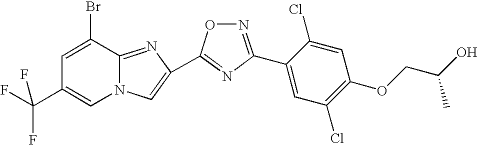 | (2R)-1-[(4-{5-[8-bromo-6- (trifluoromethyl)imidazo[1,2-a]pyridin-2-yl]- 1,2,4-oxadiazol-3-yl}-2,5- dichlorophenyl)oxy]propan-2-ol |
| 313 | 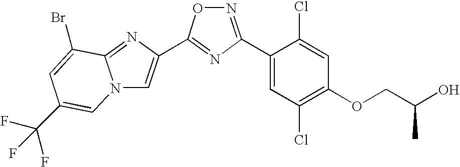 | (2S)-1-[(4-{5-[8-bromo-6- (trifluoromethyl)imidazo[1,2-a]pyridin-2-yl]- 1,2,4-oxadiazol-3-yl}-2,5- dichlorophenyl)oxy]propan-2-ol |
| 314 | 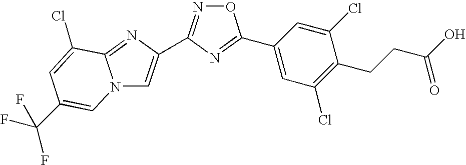 | 3-(2,6-dichloro-4-{3-[8-chloro-6- (trifluoromethyl)imidazo[1,2-a]pyridin-2-yl]- 1,2,4-oxadiazol-5-yl}phenyl)propanoic acid |
| 315 | 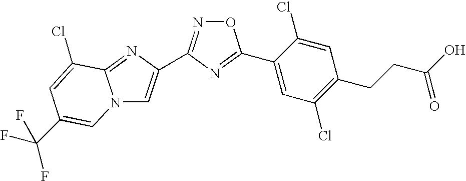 | 3-(2,5-dichloro-4-{3-[8-chloro-6- (trifluoromethyl)imidazo[1,2-a]pyridin-2-yl]- 1,2,4-oxadiazol-5-yl}phenyl)propanoic acid |
| 316 | 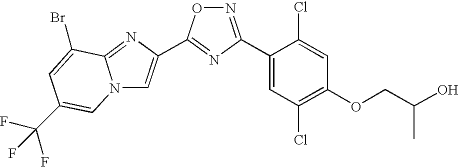 | 1-[(4-{5-[8-bromo-6- (trifluoromethyl)imidazo[1,2-a]pyridin-2-yl]- 1,2,4-oxadiazol-3-yl}-2,5- dichlorophenyl)oxy]propan-2-ol |
| 317 | 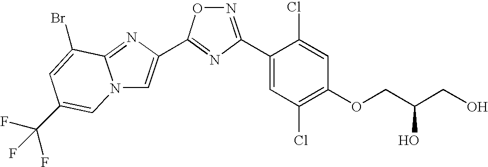 | (2R)-3-[(4-{5-[8-bromo-6- (trifluoromethyl)imidazo[1,2-a]pyridin-2-yl]- 1,2,4-oxadiazol-3-yl}-2,5- dichlorophenyl)oxy]propane-1,2-diol |
| 318 | 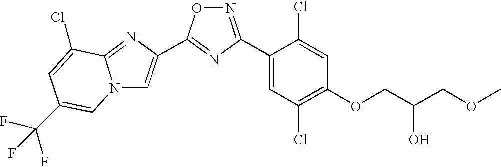 | 1-[(2,5-dichloro-4-{5-[8-chloro-6- (trifluoromethyl)imidazo[1,2-a]pyridin-2-yl]- 1,2,4-oxadiazol-3-yl}phenyl)oxy]-3- (methyloxy)propan-2-ol |
| 319 | 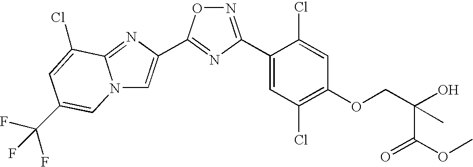 | methyl 3-[(2,5-dichloro-4-{5-[8-chloro-6- (trifluoromethyl)imidazo[1,2-a]pyridin-2-yl]- 1,2,4-oxadiazol-3-yl}phenyl)oxy]-2-hydroxy-2- methylpropanoate |
| 320 | 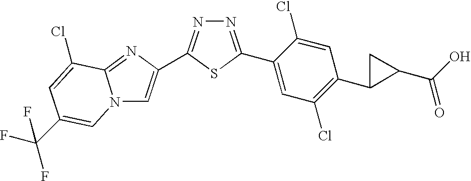 | 2-(2,5-dichloro-4-{5-[8-chloro-6- (trifluoromethyl)imidazo[1,2-a]pyridin-2-yl]- 1,3,4-thiadiazol-2- yl}phenyl)cyclopropanecarboxylic acid |
| 321 | 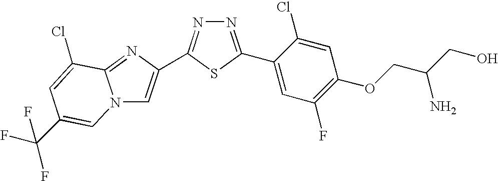 | 2-amino-3-[(5-chloro-4-{5-[8-chloro-6- (trifluoromethyl)imidazo[1,2-a]pyridin-2-yl]- 1,3,4-thiadiazol-2-yl}-2- fluorophenyl)oxy]propan-1-ol |
| 322 | 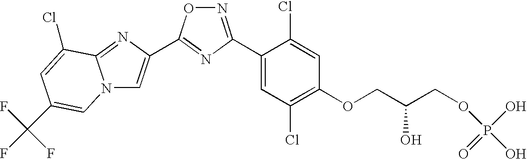 | (2R)-3-[(2,5-dichloro-4-{5-[8-chloro-6- (trifluoromethyl)imidazo[1,2-a]pyridin-2-yl]- 1,2,4-oxadiazol-3-yl}phenyl)oxy]-2- hydroxypropyl dihydrogen phosphate |
| 323 | 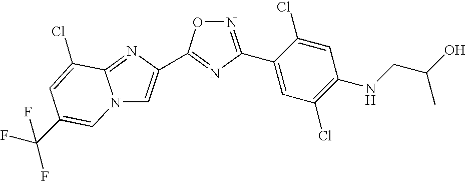 | 1-[(2,5-dichloro-4-{5-[8-chloro-6- (trifluoromethyl)imidazo[1,2-a]pyridin-2-yl]- 1,2,4-oxadiazol-3-yl}phenyl)amino]propan-2-ol |
| 324 | 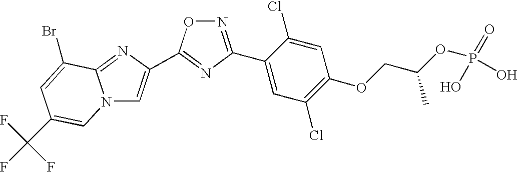 | (1R)-2-[(4-{5-[8-bromo-6- (trifluoromethyl)imidazo[1,2-a]pyridin-2-yl]- 1,2,4-oxadiazol-3-yl}-2,5-dichlorophenyl)oxy]-1- methylethyl dihydrogen phosphate |
| 325 | 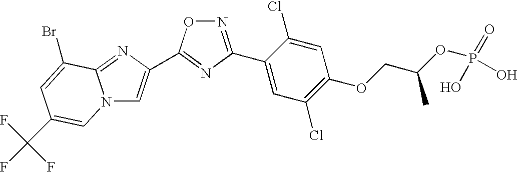 | (1S)-2-[(4-{5-[8-bromo-6- (trifluoromethyl)imidazo[1,2-a]pyridin-2-yl]- 1,2,4-oxadiazol-3-yl}-2,5-dichlorophenyl)oxy]-1- methylethyl dihydrogen phosphate |
| 326 | 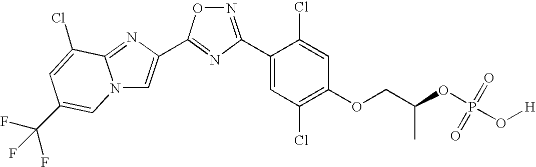 | (1S)-2-[(2,5-dichloro-4-{5-[8-chloro-6- (trifluoromethyl)imidazo[1,2-a]pyridin-2-yl]- 1,2,4-oxadiazol-3-yl}phenyl)oxy]-1-methylethyl hydrogen sulfate |
| 327 | 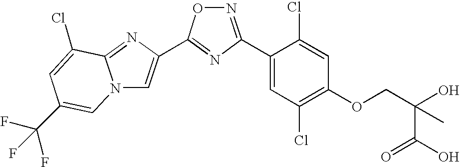 | 3-[(2,5-dichloro-4-{5-[8-chloro-6- (trifluoromethyl)imidazo[1,2-a]pyridin-2-yl]- 1,2,4-oxadiazol-3-yl}phenyl)oxy]-2-hydroxy-2- methylpropanoic acid |
| 328 | 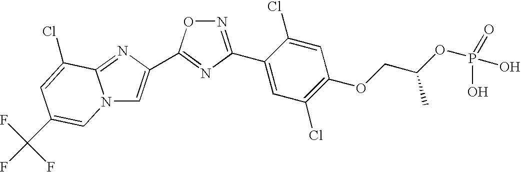 | (1R)-2-[(2,5-dichloro-4-{5-[8-chloro-6- (trifluoromethyl)imidazo[1,2-a]pyridin-2-yl]- 1,2,4-oxadiazol-3-yl}phenyl)oxy]-1-methylethyl dihydrogen phosphate |
| 329 | 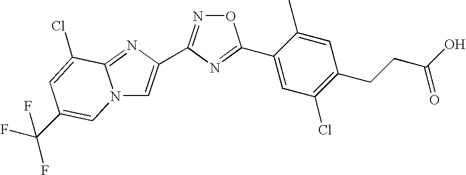 | 3-(2-chloro-4-{3-[8-chloro-6- (trifluoromethyl)imidazo[1,2-a]pyridin-2-yl]- 1,2,4-oxadiazol-5-yl}-5-methylphenyl)propanoic acid |
| 330 | 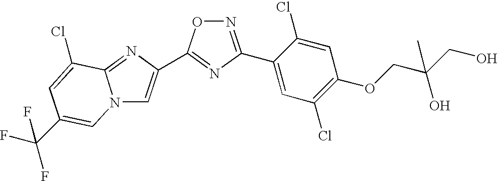 | 3-[(2,5-dichloro-4-{5-[8-chloro-6- (trifluoromethyl)imidazo[1,2-a]pyridin-2-yl]- 1,2,4-oxadiazol-3-yl}phenyl)oxy]-2- methylpropane-1,2-diol |
| 331 | 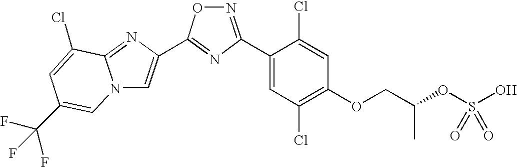 | (1R)-2-[(2,5-dichloro-4-{5-[8-chloro-6- (trifluoromethyl)imidazo[1,2-a]pyridin-2-yl]- 1,2,4-oxadiazol-3-yl}phenyl)oxy]-1-methylethyl hydrogen sulfate |
| 332 | 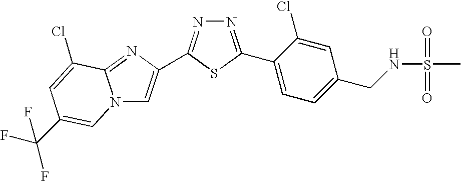 | N-[(3-chloro-4-{5-[8-chloro-6- (trifluoromethyl)imidazo[1,2-a]pyridin-2-yl]- 1,3,4-thiadiazol-2- yl}phenyl)methyl]methanesulfonamide |
| 333 | 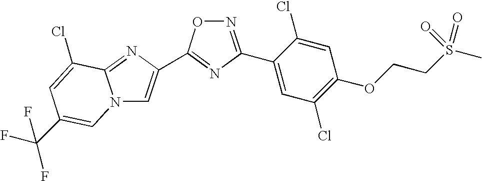 | 8-chloro-2-[3-(2,5-dichloro-4-{[2- (methylsulfonyl)ethyl]oxy}phenyl)-1,2,4- oxadiazol-5-yl]-6-(trifluoromethyl)imidazo[1,2- a]pyridine |
| 334 | 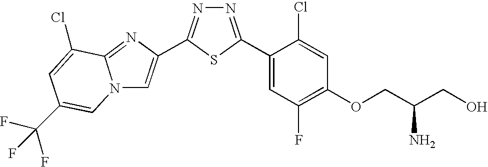 | (2R)-2-Amino-3-[(5-chloro-4-{5-[8-chloro-6- (trifluoromethyl)imidazo[1,2-a]pyridin-2-yl]- 1,3,4-thiazol-2-yl}-2- fluorophenyl)oxy]propan-1-ol |
| 335 | 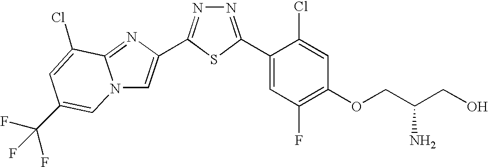 | (2S)-2-Amino-3-[(5-chloro-4-{5-[8-chloro-6- (trifluoromethyl)imidazo[1,2-a]pyridin-2-yl]- 1,3,4-thiadiazol-2-yl}-2- fluorophenyl)oxy]propan-1-ol |
| 336 | 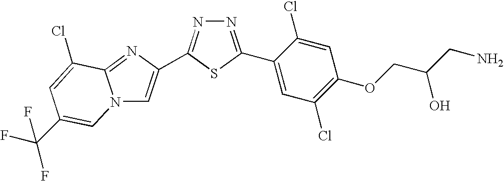 | 1-amino-3-[(2,5-dichloro-4-{5-[8-chloro-6- (trifluoromethyl)imidazo[1,2-a]pyridin-2-yl]- 1,3,4-thiadiazol-2-yl}phenyl)oxy]propan-2-ol |
| 337 | 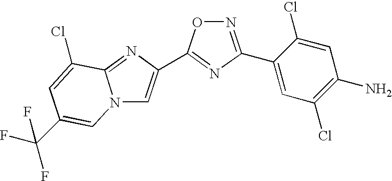 | 2,5-dichloro-4-{5-[8-chloro-6- (trifluoromethyl)imidazo[1,2-a]pyridin-2-yl]- 1,2,4-oxadiazol-3-yl}aniline |
| 338 | 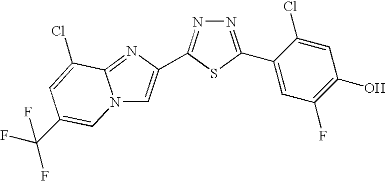 | 5-chloro-4-{5-[8-chloro-6- (trifluoromethyl)imidazo[1,2-a]pyridin-2-yl]- 1,3,4-thiadiazol-2-yl}-2-fluorophenol |
| 339 | 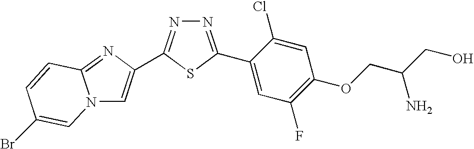 | 2-amino-3-({4-[5-(6-bromoimidazo[1,2-a]pyridin- 2-yl)-1,3,4-thiadiazol-2-yl]-5-chloro-2- fluorophenyl}oxy)propan-1-ol |
| 340 | 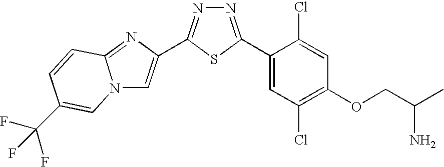 | 1-[(2,5-dichloro-4-{5-[6- (trifluoromethyl)imidazo[1,2-a]pyridin-2-yl]- 1,3,4-thiadiazol-2-yl}phenyl)oxy]propan-2-amine |
| 341 | 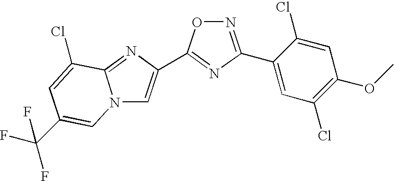 | 5-(8-chloro-6-(trifluoromethyl)imidazol(1,2- a)pyridine-2-yl-3-(2,5-dichloro-4- methoxyphenyl)-1,2,4-oxadizole |
| 342 | 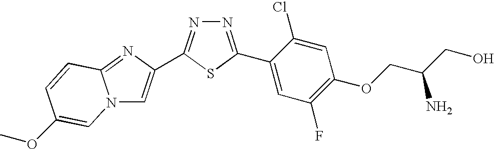 | (2R)-2-amino-3-[(5-chloro-2-fluoro-4-{5-[6- (methyloxy)imidazo[1,2-a]pyridin-2-yl]-1,3,4- thiadiazol-2-yl}phenyl)oxy]propan-1-ol |
| 420 | 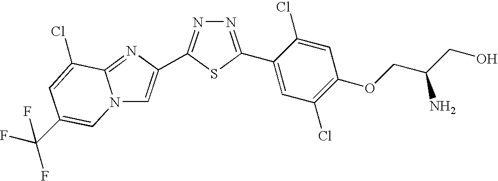 | (2R)-2-amino-3-[(2,5-dichloro-4-{5-[8-chloro-6- (trifluoromethyl)imidazo[1,2-a]pyridin-2-yl]- 1,3,4-thiadiazol-2-yl}phenyl)oxy]propan-1-ol |
| 421 | 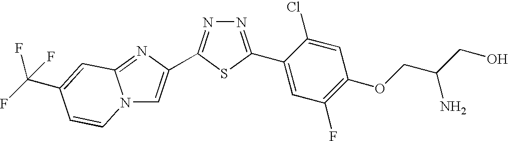 | 2-amino-3-[(5-chloro-2-fluoro-4-{5-[7- (trifluoromethyl)imidazo[1,2-a]pyridin-2-yl]- 1,3,4-thiadiazol-2-yl}phenyl)oxy]propan-1-ol |

The following compounds of the invention were prepared and are useful intermediates in the synthesis of other compounds of the invention.

TABLE 2
Compounds of the Invention Useful in the Synthesis of Other Compounds of the
Invention

| Entry | | |
|---|---|---|
| No. | Structure | Name |
| 343 | 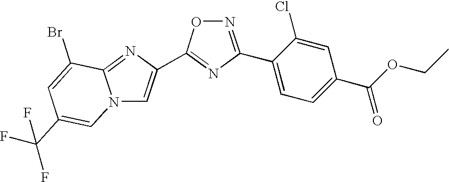 | ethyl 4-(5-(8-bromo-6- (trifluoromethyl)imidazo[1,2-a]pyridin-2-yl)- 1,2,4-oxadiazol-3-yl)-3-chlorobenzoate |
| 344 | 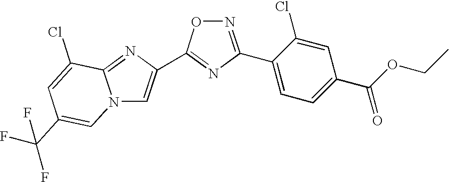 | ethyl 4-(5-(8-chloro-6- (trifluoromethyl)imidazo[1,2-a]pyridin-2-yl)- 1,2,4-oxadiazol-3-yl)-3-chlorobenzoate |
| 345 | 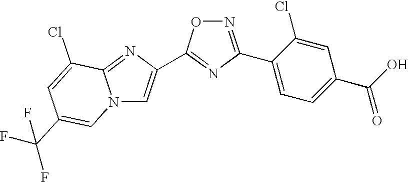 | 3-chloro-4-(5-(8-chloro-6- (trifluoromethyl)imidazo[1,2-a]pyridin-2-yl)- 1,2,4-oxadiazol-3-yl) benzoic acid |
| 346 | 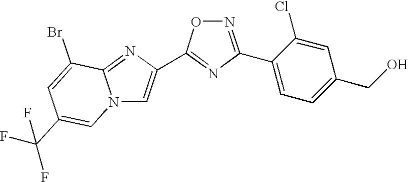 | (4-(5-(8-bromo-6- (trifluoromethyl)imidazo[1,2-a]pyridin-2-yl)- 1,2,4-oxadiazol-3-yl)-3- chlorophenyl)methanol |
| 347 | 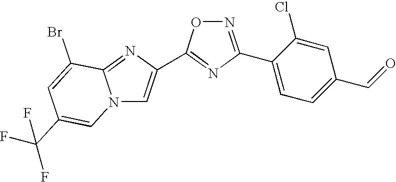 | 4-(5-(8-bromo-6- (trifluoromethyl)imidazo[1,2-a]pyridin-2-yl)- 1,2,4-oxadiazol-3-yl)-3-chlorobenzaldehyde |
| 348 | 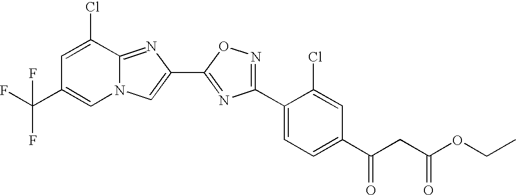 | ethyl 3-(3-chloro-4-(5-(8-chloro-6- (trifluoromethyl)imidazo[1,2-a]pyridin-2-yl)- 1,2,4-oxadiazol-3-yl)phenyl)-3- oxopropanoate |
| 349 | 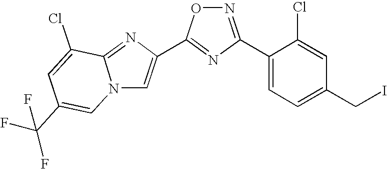 | 3-(2-chloro-4-(iodomethyl)phenyl)-5-(8- chloro-6-(trifluoromethyl)imidazo[1,2-a] pyridin-2-yl)-1,2,4-oxadiazole |
| 350 | 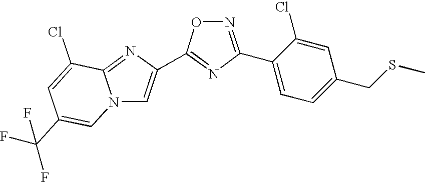 | 3-(2-chloro-4-(methylthiomethyl)phenyl)-5- (8-chloro-6-(trifluoromethyl) imidazo[1,2- a]pyridin-2-yl)-1,2,4-oxadiazole |
| 351 | 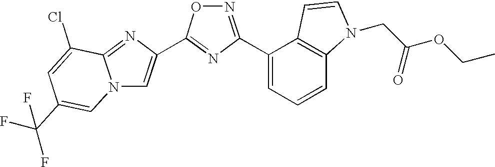 | ethyl [2-(4-(5-(8-chloro-6- (trifluoromethyl)imidazo[1,2-a]pyridin-2-yl)- 1,2,4-oxadiazol-3-yl)-1H-indol-1-yl)]acetate |
| 352 | 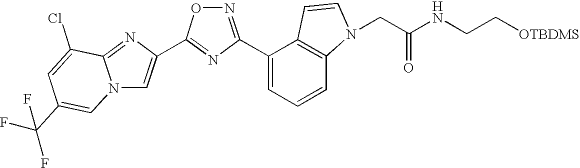 | N-(2-(tert-butyldimethylsilyloxy)ethyl)-2-(4- (5-(8-chloro-6-trifluoromethyl) imidazo[1,2- a]pyridin-2-yl)-1,2,4-oxadiazol-3-yl)-1H- indol-1-yl)acetamide |
| 353 | 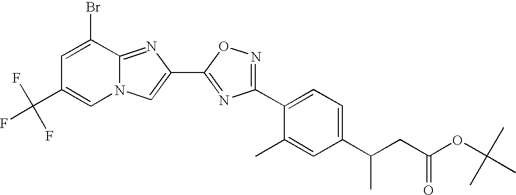 | tert-butyl [3-(4-(5-(8-bromo-6- (trifluoromethyl)imidazo[1,2-a]pyridin-2-yl)- 1,2,4-oxadiazol-3-yl)-3-methylphenyl)] butanoate |
| 354 | 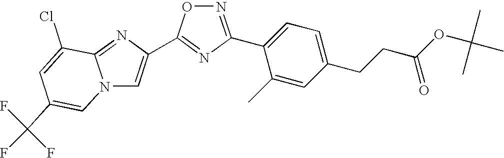 | tert-butyl 3-(3-methyl-4-(5-(8-chloro-6- (trifluoromethyl)-imidazo[1,2-a]pyridin-2-yl)- 1,2,4-oxadiazol-3-yl)phenyl)propanoate |
| 355 | 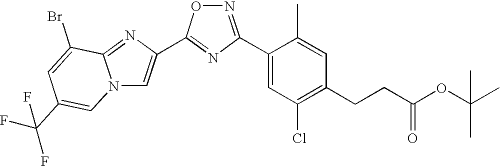 | tert-butyl 3-(4-(5-(8-bromo-6- (trifluoromethyl)imidazo[1,2-a]pyridin-2-yl)- 1,2,4-oxadiazol-3-yl)-2-chloro-5- methylphenyl) propanoate |
| 356 | 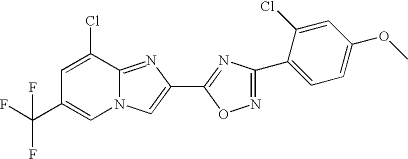 | 3-(2-chloro-4-methoxyphenyl)-5-(8-chloro-6- (trifluoromethyl)imidazo[1,2-a]pyridin-2-yl)- 1,2,4-oxadiazole |
| 357 | 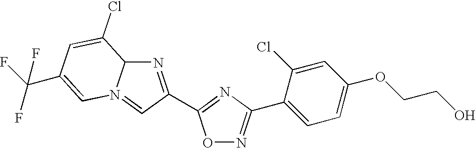 | 2-(3-chloro-4-(5-(8-chloro-6- (trifluoromethyl)imidazo[1,2-a]pyridin-2-yl)- 1,2,4-oxadiazol-3-yl)phenoxy)ethanol |
| 358 | 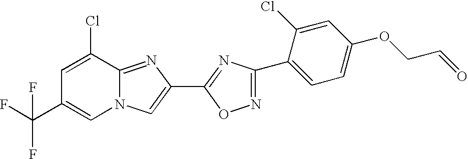 | 2-(3-chloro-4-(5-(8-chloro-6- (trifluoromethyl)imidazo[1,2-a]pyridin-2-yl)- 1,2,4-oxadiazol-3-yl)phenoxy)acetaldehyde |
| 359 | 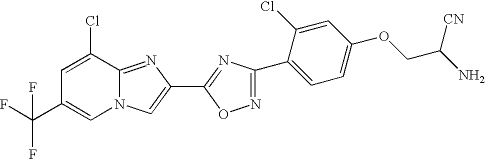 | 2-amino-3-(3-chloro-4-(5-(8-chloro-6- (trifluoromethyl)imidazo[1,2-a]pyridin-2-yl)- 1,2,4-oxadiazol-3-yl)phenoxy)propanenitrile |
| 360 | 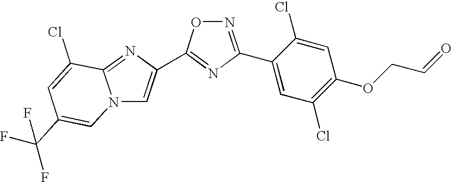 | 2-(2,5-dichloro-4-(5-(8-chloro-6- (trifluoromethyl)imidazo[1,2-a]pyridin-2-yl)- 1,2,4-oxadiazol-3-yl)phenoxy)acetaldehyde |
| 361 | 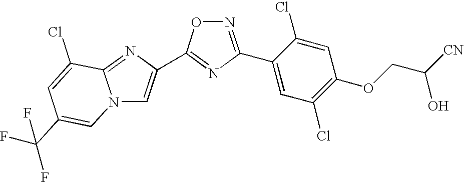 | 3-(2,5-dichloro-4-(5-(8-chloro-6- (trifluoromethyl)imidazo[1,2-a]pyridin-2-yl)- 1,2,4-oxadiazol-3-yl)phenoxy)-2- hydroxypropanenitrile |
| 362 | 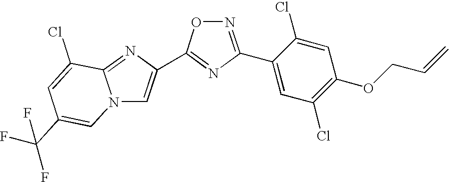 | 3-(4-(allyloxy)-2,5-dichlorophenyl)-5-(8- chloro-6-(trifluoromethyl)imidazo[1,2- a]pyridin-2-yl)-1,2,4-oxadiazole |
| 363 | 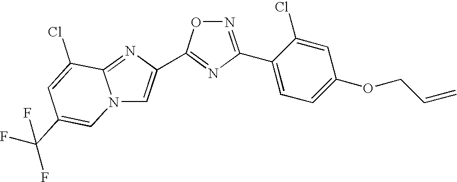 | 3-(4-(allyloxy)-2-chlorophenyl)-5-(8-chloro- 6-(trifluoromethyl)imidazo[1,2-a]pyridin-2- yl)-1,2,4-oxadiazole |
| 364 | 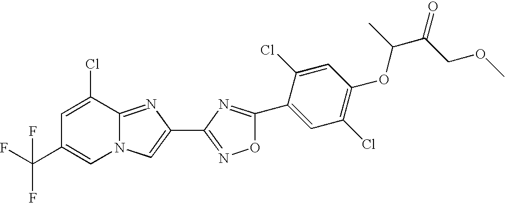 | 2-(5-chloro-4-(5-(8-chloro-6- (trifluoromethyl)imidazo[1,2-a]pyridin-2-yl)- 1,2,4-oxadiazol-3-yl)-2- fluorophenoxy)ethanol |
| 365 | 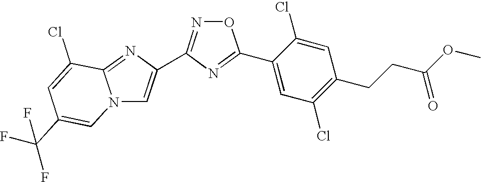 | methyl 3-(2,5-dichloro-4-(3-(8-chloro-6- (trifluoromethyl)imidazo[1,2-a]pyridine-2- yl)-1,2,4-oxadiazol-5-yl)phenyl)propanoate |
| 366 | 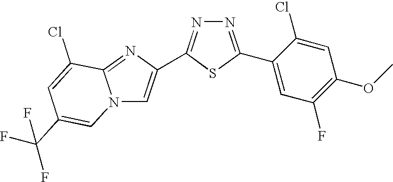 | 2-(2-chloro-5-fluoro-4-methoxyphenyl)-5-(8- chloro-6-(trifluoromethyl)imidazo[1,2- a]pyridin-2-yl)-1,3,4-thiadiazole |
| 367 | 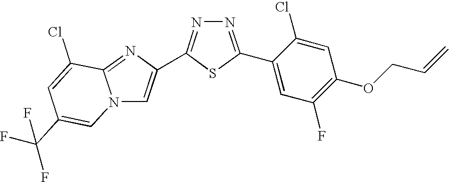 | 2-(4-(allyloxy)-2-chloro-5-fluorophenyl)-5-(8- chloro-6-(trifluoromethyl)imidazo[1,2- a]pyridin-2-yl)-1,3,4-thiadiazole |
| 368 | 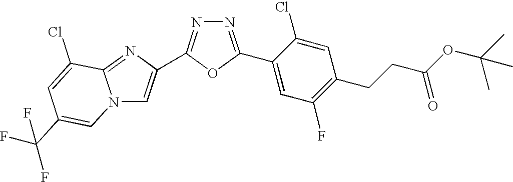 | 3-(5-chloro-4-(5-(8-chloro-6- (trifluoromethyl)imidazo(1,2-a]pyridine-2- yl)-1,3,4-oxadiazol-2-yl)-2- fluorophenyl)propanoic acid |
| 369 | 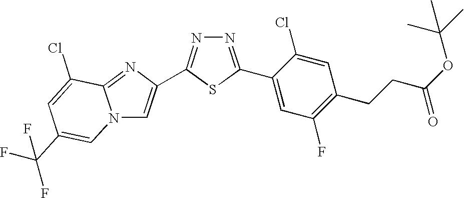 | tert-butyl 3-(5-chloro-4-(5-(8-chloro-6- (trifluoromethyl)imidazo[1,2-a]pyridin-2-yl)- 1,3,4-thiadiazol-2-yl)-2- fluorophenyl)propanoate |
| 370 | 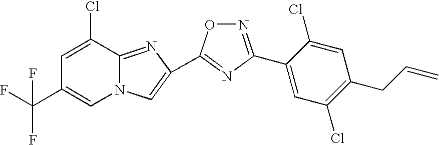 | 3-(4-allyl-2,5-dichlorophenyl)-5-(8-chloro-6- (trifluoromethyl)imidazo[1,2-a]pyridin-2-yl)- 1,2,4-oxadiazole |
| 371 | 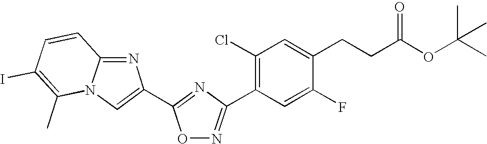 | tert-butyl 3-(5-chloro-2-fluoro-4-(5-(6-iodo- 5-methylimidazo[1,2-a]pyridin-2-yl)-1,2,4- oxadiazol-3-yl)phenyl)propanoate |
| 372 | 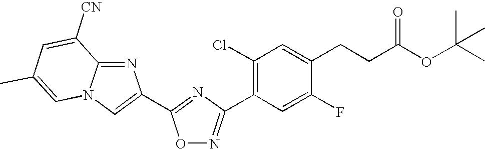 | tert-butyl 3-(5-chloro-4-(5-(8-cyano-6- methylimidazo[1,2-a]pyridin-2-yl)-1,2,4- oxadiazol-3-yl)-2-fluorophenyl)propanoate |
| 373 | 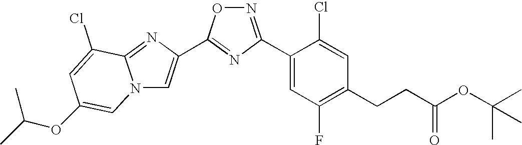 | tert-butyl 3-(5-chloro-4-(5-(8-chloro-6- isopropoxyimidazo[1,2-a]pyridin-2-yl)-1,2,4- oxadiazol-3-yl)-2-fluorophenyl)propanoate |
| 374 | 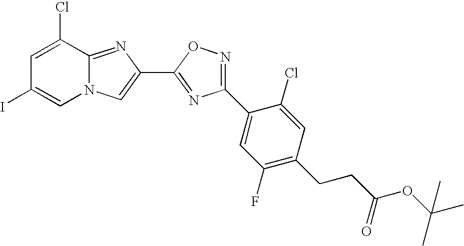 | tert-butyl 3-(5-chloro-4-(5-(8-chloro-6- iodoimidazo[1,2-a]pyridin-2-yl)-1,2,4- oxadiazol-3-yl)-2-fluorophenyl)propanoate |
| 375 | 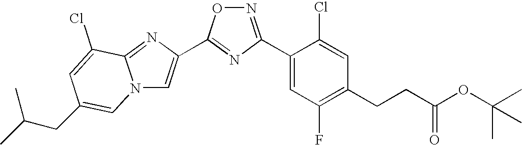 | tert-butyl 3-(5-chloro-4-(5-(8-chloro-6- isobutylimidazo[1,2-a]pyridin-2-yl)-1,2,4- oxadiazol-3-yl)-2-fluorophenyl)propanoate |
| 376 | 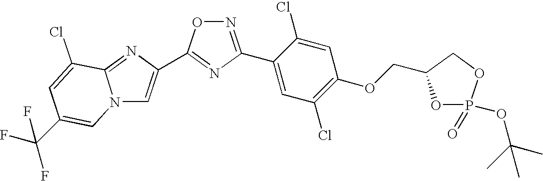 | 8-chloro-2-(3-{4-[({(4S)-2-[(1,1- dimethylethyl)oxy]-2-oxido-1,3,2- dioxaphospholan-4-yl}methyl)oxy]-2,5- dimethylphenyl}-1,2,4-oxadiazol-5-yl)-6- (trifluoromethyl)imidazo[1,2-a]pyridine |
| 377 | 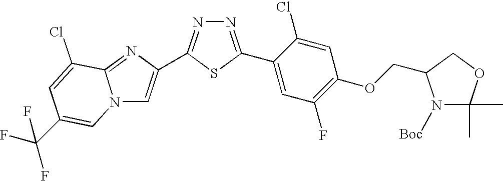 | tert-butyl 4-((5-chloro-4-(5-(8-chloro-6- (trifluoromethyl)imidazo[1,2-a]pyridin-2-yl)- 1,3,4-thiadiazol-2-yl)-2- fluorophenoxy)methyl)-2,2- dimethyloxazolidine-3-carboxylate |
| 378 | 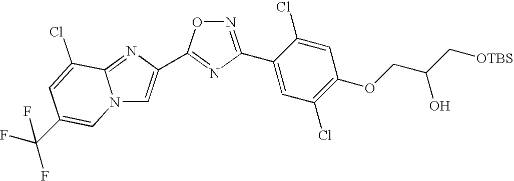 | 1-(tert-butyldimethylsilyloxy)-3-(2,5- dichloro-4-(5-(8-chloro-6- (trifluoromethyl)imidazo[1,2-a]pyridin-2-yl)- 1,2,4-oxadiazol-3-yl)phenoxy)propan-2-ol |
| 379 | 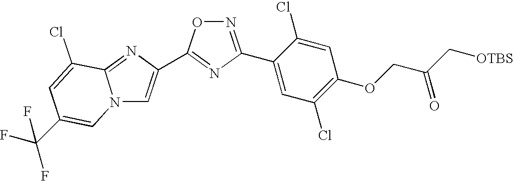 | 1-(tert-butyldimethylsilyloxy)-3-(2,5- dichloro-4-(5-(8-chloro-6- (trifluoromethyl)imidazo[1,2-a]pyridin-2-yl)- 1,2,4-oxadiazol-3-yl)phenoxy)propan-2-one |
| 380 | 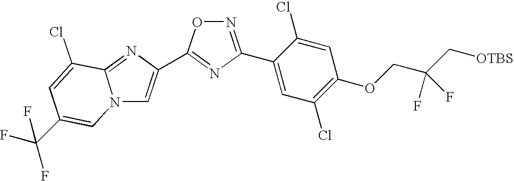 | 3-(4-(3-(tert-butyldimethylsilyloxy)-2,2- difluoropropoxy)-2,5-dichlorophenyl)-5-(8- chloro-6-(trifluoromethyl)imidazo[1,2- a]pyridin-2-yl)-1,2,4-oxadiazole |
| 381 | 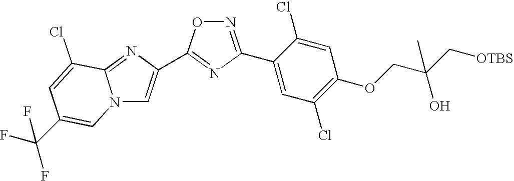 | 1-(tert-butyldimethylsilyloxy)-3-(2,5- dichloro-4-(5-(8-chloro-6- (trifluoromethyl)imidazo[1,2-a]pyridin-2-yl)- 1,2,4-oxadiazol-3-yl)phenoxy)-2- methylpropan-2-ol |
| 382 | 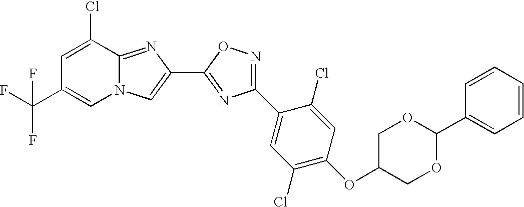 | 5-(8-chloro-6-(trifluoromethyl)imidazo[1,2- a]pyridin-2-yl)-3-(2,5-dichloro-4-(2-phenyl- 1,3-dioxan-5-yloxy)phenyl)-1,2,4-oxadiazole |
| 383 | 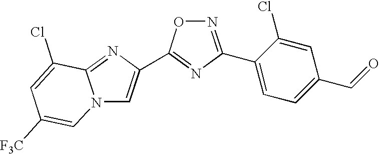 | 4-(5-(8-chloro-6- (trifluoromethyl)imidazo[1,2-a]pyridin-2-yl)- 1,2,4-oxadiazol-3-yl)-3-chlorobenzaldehyde |
| 384 | 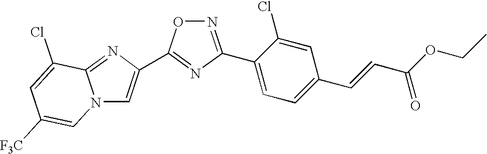 | (E)-ethyl 3-(3-chloro-4-(5-(8-chloro-6- (trifluoromethyl)imidazo[1,2-a]pyridin-2-yl)- 1,2,4-oxadiazol-3-yl)phenyl)acrylate |
| 385 | 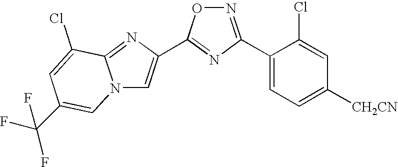 | 2-(3-chloro-4-(5-(8-chloro-6- (trifluoromethyl)imidazo[1,2-a]pyridin-2-yl)- 1,2,4-oxadiazol-3-yl)phenyl)acetonitrile |
| 386 | 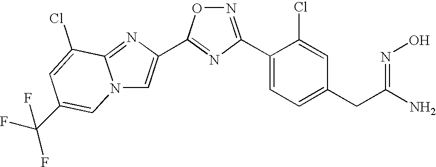 | 2-(3-chloro-4-(5-(8-chloro-6- (trifluoromethyl)imidazo[1,2-a]pyridin-2-yl)- 1,2,4-oxadiazo1-3-yl)phenyl)-N′- hydroxyacetimidamide |
| 387 | 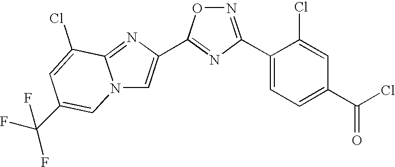 | 3-chloro-4-(5-(8-chloro-6- (trifluoromethyl)imidazo[1,2-a]pyridin-2-yl)- 1,2,4-oxadiazol-3-yl)benzoyl chloride |
| 388 | 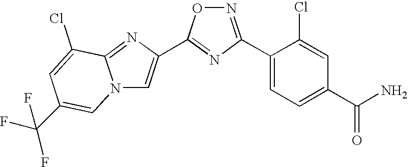 | 3-chloro-4-(5-(8-chloro-6- (trifluoromethyl)imidazo[1,2-a]pyridin-2-yl)- 1,2,4-oxadiazol-3-yl)benzamide |
| 389 | 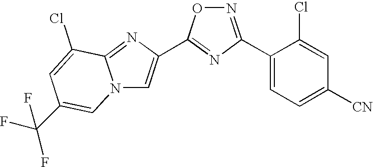 | 3-chloro-4-(5-(8-chloro-6- (trifluoromethyl)imidazo[1,2-a]pyridin-2-yl)- 1,2,4-oxadiazol-3-yl)benzonitrile |
| 390 | 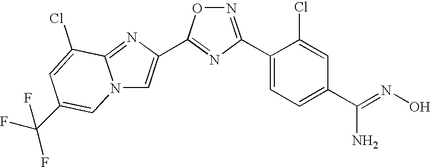 | 3-chloro-4-(5-(8-chloro-6- (trifluoromethyl)imidazo[1,2-a]pyridin-2-yl)- 1,2,4-oxadiazo1-3-yl)-N′- hydroxybenzimidamide |
| 391 | 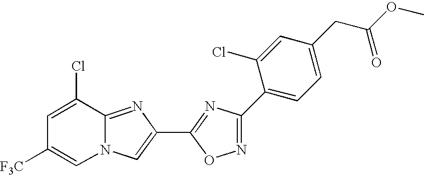 | methyl 2-(3-chloro-4-(5-(8-chloro-6- (trifluoromethyl)imidazo[1,2-a]pyridin-2-yl)- 1,2,4-oxadiazol-3-yl)phenyl)acetate |
| 392 | 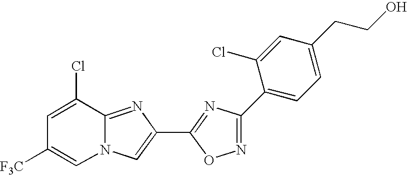 | 2-(3-chloro-4-(5-(8-chloro-6- (trifluoromethyl)imidazo[1,2-a]pyridin-2-yl)- 1,2,4-oxadiazol-3-yl)phenyl)ethanol |
| 393 | 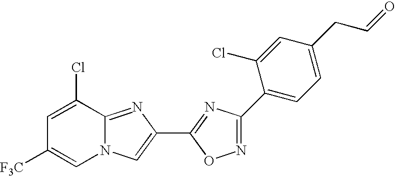 | 2-(3-chloro-(5-(8-chloro-6- (trifluoromethyl)imidazo[1,2-a]pyridin-2-yl)- 1,2,4-oxadiazol-3-yl)phenyl)acetaldehyde |
| 394 | 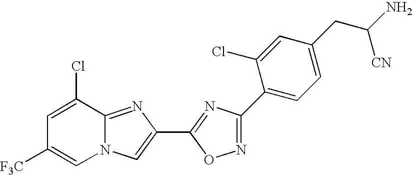 | 2-amino-3-(3-chloro-4-(5-(8-chloro-6- (trifluoromethyl)imidazo[1,2-a]pyridin-2-yl)- 1,2,4-oxadiazol-3-yl)phenyl)propanenitrile |
| 395 | 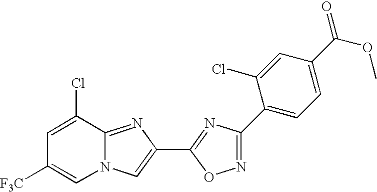 | methyl 4-(5-(8-chloro-6- (trifluoromethyl)imidazo[1,2-a]pyridin-2-yl)- 1,2,4-oxadiazol-3-yl)-3-chlorobenzoate |
| 396 | 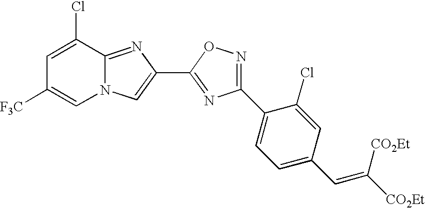 | diethyl 2-(3-chloro-4-(5-(8-chloro-6- (trifluoromethyl)imidazo[1,2-a]pyridin-2-yl)- 1,2,4-oxadiazol-3-yl)benzylidene)malonate |
| 397 | 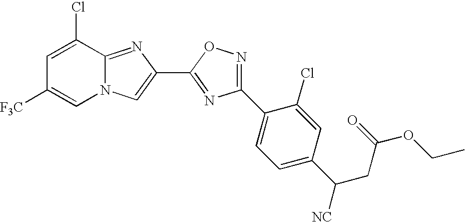 | ethyl 3-(3-chloro-4-(5-(8-chloro-6- (trifluoromethyl)imidazo[1,2-a]pyridin-2-yl)- 1,2,4-oxadiazol-3-yl)phenyl)-3- cyanopropanoate |
| 398 | 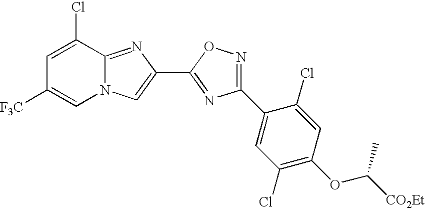 | (R)-ethyl 2-(2,5-dichloro-4-(5-(8-chloro-6- (trifluoromethyl)imidazo[1,2-a]pyridin-2-yl)- 1,2,4-oxadiazol-3-yl)phenoxy)propanoate |
| 399 | 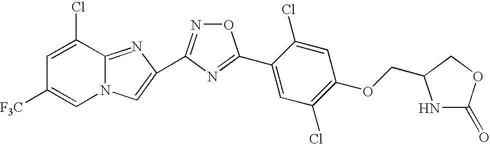 | 4-((2,5-Dichloro-4-(3-(8-chloro-6- (trifluoromethyl)imidazo[1,2-a]pyridin-2-yl)- 1,2,4-oxadiazol-5- yl)phenoxy)methyl)oxazolidin-2-one |
| 400 | 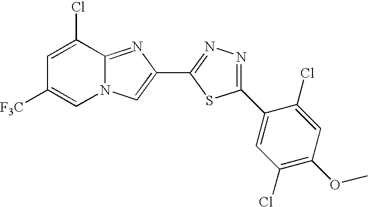 | 2-(8-chloro-6-(trifluoromethyl)imidazo[1,2- a]pyridin-2-yl)-5-(2,5-dichloro-4- methoxyphenyl)-1,3,4-thiadiazole |
| 401 | 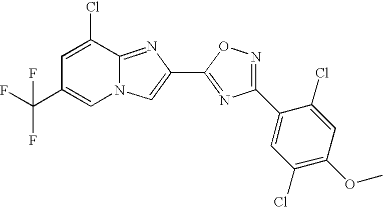 | 5-(8-chloro-6-(trifluoromethyl)imidazo[1,2- a]pyridin-2-yl)-3-(2,5-dichloro-4- methoxyphenyl)-1,2,4-oxadiazole |
| 402 | 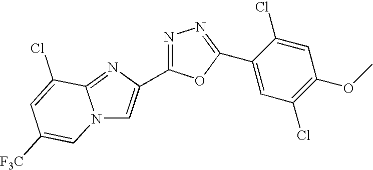 | 2-(8-chloro-6-(trifluoromethyl)imidazo[1,2- a]pyridin-2-yl)-5-(2,5-dichloro-4- methoxyphenyl)-1,3,4-oxadiazole |
| 403 | 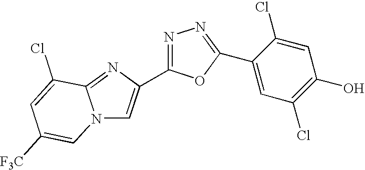 | 2,5-dichloro-4-(5-(8-chloro-6- (trifluoromethyl)imidazo[1,2-a]pyridin-2-yl)- 1,3,4-oxadiazol-2-yl)phenol |
| 404 | 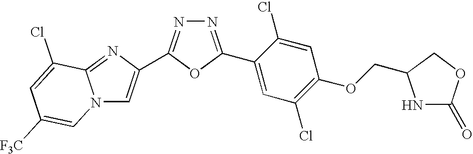 | 4-((2,5-dichloro-4-(5-(8-chloro-6- (trifluoromethyl)imidazo[1,2-a]pyridin-2-yl)- 1,3,4-oxadiazol-2- yl)phenoxy)methyl)oxazolidin-2-one |
| 405 | 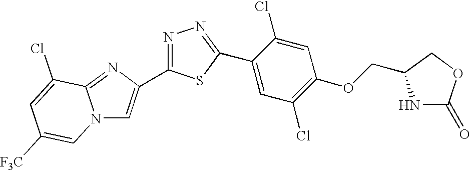 | (R)-4-((2,5-dichloro-4-(5-(8-chloro-6- (trifluoromethyl)imidazo[1,2-a]pyridin-2-yl)- 1,3,4-thiadiazol-2- yl)phenoxy)methyl)oxazolidin-2-one |
| 406 | 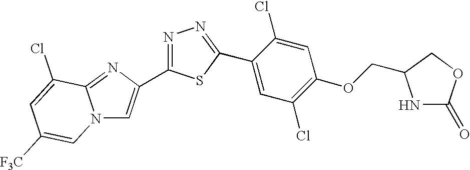 | 4-((2,5-dichloro-4-(5-(8-chloro-6- (trifluoromethyl)imidazo[1,2-a]pyridin-2-yl)- 1,3,4-thiadiazol-2- yl)phenoxy)methyl)oxazolidin-2-one |
| 407 | 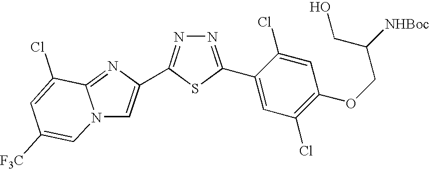 | tert-butyl 1-(5-chloro-4-(5-(8-chloro-6- (trifluoromethyl)imidazo[1,2-a]pyridin-2-yl)- 1,3,4-thiadiazol-2-yl)-2-fluorophenoxy)-3- (di-tert-butoxyphosphoryloxy)propan-2- ylcarbamate |
| 408 | 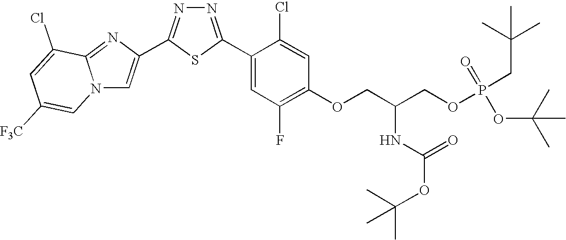 | tert-butyl 1-(5-chloro-4-(5-(8-chloro-6- (trifluoromethyl)imidazo[1,2-a]pyridin-2-yl)- 1,3,4-thiadiazol-2-yl)-2-fluorophenoxy)-3- (di-tert-butoxyphosphoryloxy)propan-2- ylcarbamate |
| 409 | 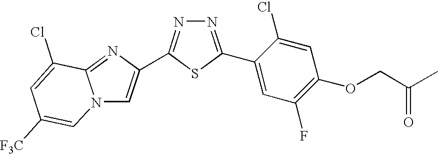 | 1-(5-chloro-4-(5-(8-chloro-6- (trifluoromethyl)imidazo[1,2-a]pyridin-2-yl)- 1,3,4-thiadiazol-2-yl)-2- fluorophenoxy)propan-2-one |
| 410 | 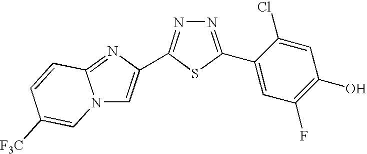 | 5-chloro-2-fluoro-4-(5-(6- (trifluoromethyl)imidazo[1,2-a]pyridin-2-yl)- 1,3,4-thiadiazol-2-yl)phenol |
| 411 | 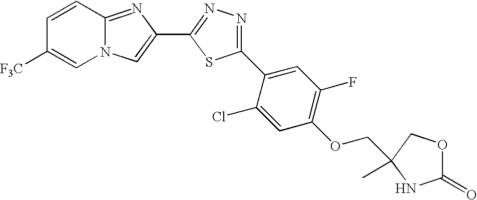 | 4-((5-chloro-2-fluoro-4-(5-(6- (trifluoromethyl)imidazo[1,2-a]pyridin-2-yl)- 1,3,4-thiadiazol-2-yl)phenoxy)methyl)-4- methyloxazolidin-2-one |
| 412 | 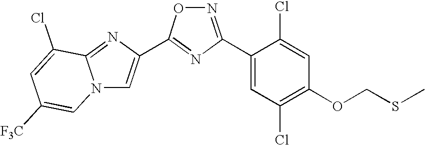 | 5-(8-chloro-6-(trifluoromethyl)imidazo[1,2- a]pyridin-2-yl)-3-(2,5-dichloro-4- (methylthiomethoxy)phenyl)-1,2,4-oxadiazole |
| 413 | 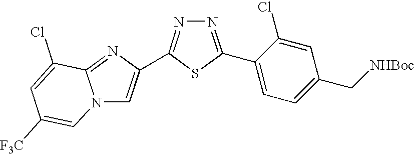 | tert-butyl 3-chloro-4-(5-(8-chloro-6- (trifluoromethyl)imidazo[1,2-a]pyridin-2-yl)- 1,3,4-thiadiazol-2-yl)benzylcarbamate |
| 414 | 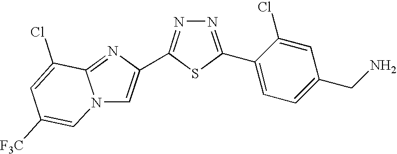 | (3-chloro-4-(5-(8-chloro-6- (trifluoromethyl)imidazo[1,2-a]pyridin-2-yl)- 1,3,4-thiadiazol-2-yl)phenyl)methanamine |
| 415 | 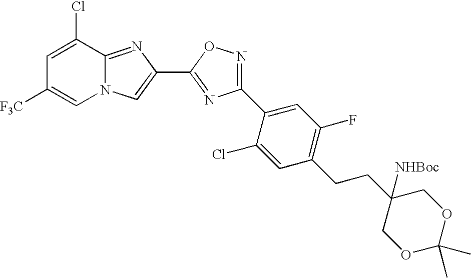 | tert-butyl 5-(5-chloro-4-(5-(8-chloro-6- (trifluoromethyl)imidazo[1,2-a]pyridin-2-yl)- 1,2,4-oxadiazol-3-yl)-2-fluorophenethyl)-2,2- dimethyl-1,3-dioxan-5-ylcarbamate |
| 416 | 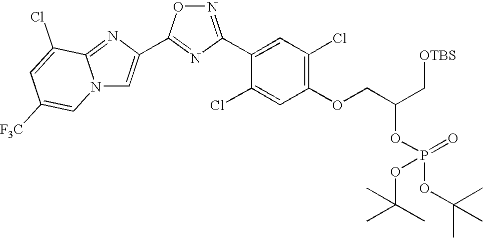 | di-tert-butyl 1-(tert-butyldimethylsilyloxy)-3- (2,5-dichloro-4-(5-(8-chloro-6- (trifluoromethyl)imidazo[1,2-a]pyridin-2-yl)- 1,2,4-oxadiazol-3-yl)phenoxy)propan-2-yl phosphate |
| 417 | 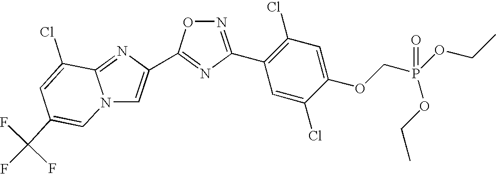 | di-tert-butyl 1-(tert-butyldimethylsilyloxy)-3- (2,5-dichloro-4-(5-(8-chloro-6- (trifluoromethyl)imidazo[1,2-a]pyridin-2-yl)- 1,2,4-oxadiazol-3 -yl)phenoxy)propan-2-yl phosphate |
| 418 | 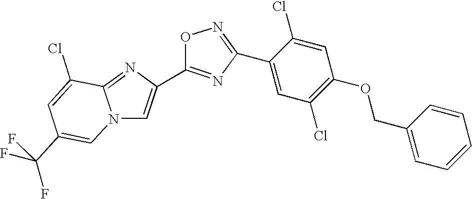 | 8-chloro-2-(3-{2,5-dichloro-4- [(phenylmethyl)oxy]phenyl}-1,2,4-oxadiazol- 5-yl)-6-(trifluoromethyl)imidazo[1,2- a]pyridine |
| 419 | 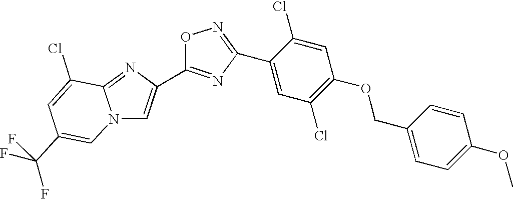 | 8-chloro-2-{3-[2,5-dichloro-4-({[4- (methyloxy)phenyl]methyl}oxy)phenyl]- 1,2,4-oxadiazol-5-yl}-6- (trifluoromethyl)imidazo[1,2-a]pyridine |

### General Administration

In one aspect, the invention provides pharmaceutical compositions comprising an agonist of S1P1 according to the invention and a pharmaceutically acceptable carrier, excipient, or diluent. In certain other specific embodiments, administration is by the oral route. Administration of the compounds of the invention, or their pharmaceutically acceptable salts, in pure form or in an appropriate pharmaceutical composition, can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, or rectally, in the form of solid, semi-solid, lyophilized powder, or liquid dosage forms, such as for example, tablets, suppositories, pills, soft elastic and hard gelatin capsules, powders, solutions, suspensions, or aerosols, or the like, specifically in unit dosage forms suitable for simple administration of precise dosages.

The compositions will include a conventional pharmaceutical carrier or excipient and a compound of the invention as the/an active agent, and, in addition, may include carriers and adjuvants, etc.

Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.]

If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylated hydroxytoluene, etc.

The choice of formulation depends on various factors such as the mode of drug administration (e.g., for oral administration, formulations in the form of tablets, pills or capsules) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

One specific route of administration is oral, using a convenient daily dosage regimen that can be adjusted according to the degree of severity of the disease-state to be treated.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid dosage forms as described above can be prepared with coatings and shells, such as enteric coatings and others well known in the art. They may contain pacifying agents, and can also be of such composition that they release the active compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., a compound(s) of the invention, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the compounds of the present invention with for example suitable non-irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

Compressed gases may be used to disperse a compound of this invention in aerosol form. Inert gases suitable for this purpose are nitrogen, carbon dioxide, fluorocarbons, and hydrofluoroalkanes, etc.

Generally, depending on the intended mode of administration, the pharmaceutically acceptable compositions will contain about 1% to about 99% by weight of a compound(s) of the invention, or a pharmaceutically acceptable salt thereof, and 99% to 1% by weight of a suitable pharmaceutical excipient. In one example, the composition will be between about 5% and about 75% by weight of a compound(s) of the invention, or a pharmaceutically acceptable salt thereof, with the rest being suitable pharmaceutical excipients.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the invention, or a pharmaceutically acceptable salt thereof, for treatment of a disease-state in accordance with the teachings of this invention.

The compounds of the invention, or their pharmaceutically acceptable salts or solvates, are administered in a therapeutically effective amount which will vary depending upon a variety of factors including the activity of the specific compound employed, the metabolic stability and length of action of the compound, the age, body weight, general health, sex, diet, mode and time of administration, rate of excretion, drug combination, the severity of the particular disease-states, and the host undergoing therapy. The compounds of the present invention can be administered to a patient at dosage levels in the range of about 0.1 to about 1,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors including the requirements of the patient, the severity of the condition being treated, and the pharmacological activity of the compound being used. The determination of optimum dosages for a particular patient is well known to one of ordinary skill in the art.

If formulated as a fixed dose, such combination products employ the compounds of this invention within the dosage range described above and the other pharmaceutically active agent(s) within its approved dosage range. Compounds of the instant invention may alternatively be used sequentially with known pharmaceutically acceptable agent(s) when a combination formulation is inappropriate.

### Utility

Compounds of this invention have been tested using the assays described in Biological Example 1, 2, 3, and 4 and have been determined to be S1P1 agonists. Following the examples disclosed herein, as well as that disclosed in the art, a person of ordinary skill in the art can determine the S1P1 agonist activity of a compound of this invention. Compounds of Formula I are therefore useful for treating diseases, particularly cancer in which S1P1 activity contributes to the pathology and/or symptomatology of the disease. For example, various immune-related conditions in which S1P1 activity contributes to its pathology and/or symptomatology include graft-versus host disease and autoimmune diseases such as multiple sclerosis, rheumatoid arthritis, systemic lupus erythematosis, psoriasis, Grave's disease, myasthenia gravis, Crohn's disease, and ulcerative colitis.

### General Synthesis

Compounds of this invention can be made by the synthetic procedures described below. The starting materials and reagents used in preparing these compounds are either available from commercial suppliers such as Aldrich Chemical Co. (Milwaukee, Wis.), or Bachem (Torrance, Calif.), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4^{th} Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure. The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure and over a temperature range from about −78° C. to about 150° C., more specifically from about 0° C. to about 125° C. and more specifically at about room (or ambient) temperature, e.g., about 20° C. Unless otherwise stated (as in the case of hydrogenation), all reactions are performed under an atmosphere of nitrogen.

Prodrugs can be prepared by techniques known to one skilled in the art. These techniques generally modify appropriate functional groups in a given compound. These modified functional groups regenerate original functional groups by routine manipulation or in vivo. Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

The compounds of the invention, or their pharmaceutically acceptable salts, may have asymmetric carbon atoms or quaternized nitrogen atoms in their structure. Compounds of the Invention that may be prepared through the syntheses described herein may exist as single stereoisomers and mixtures of stereoisomers. A stereoisomer has the meaning given by one of ordinary skill in the art and includes an enantiomer, a diastereomer, a geometric isomer, and a conformational isomer. All such single stereoisomers and mixtures thereof are within the scope of this invention. Some of the compounds of the invention may exist as tautomers. For example, where a ketone or aldehyde is present, the molecule may exist in the enol form; where an amide is present, the molecule may exist as the imidic acid; and where an enamine is present, the molecule may exist as an imine. All such tautomers are within the scope of the invention.

The present invention also includes N-oxide derivatives and protected derivatives of compounds of the Invention. For example, when compounds of the Invention contain an oxidizable nitrogen atom, the nitrogen atom can be converted to an N-oxide by methods well known in the art. When compounds of the Invention contain groups such as hydroxy, carboxy, thiol or any group containing a nitrogen atom(s), these groups can be protected with a suitable “protecting group” or “protective group”. A comprehensive list of suitable protective groups can be found in T. W. Greene, Protective Groups in Organic Synthesis, John Wiley & Sons, Inc. 1991, the disclosure of which is incorporated herein by reference in its entirety. The protected derivatives of compounds of the Invention can be prepared by methods well known in the art.

Methods for the preparation and/or separation and isolation of single stereoisomers from racemic mixtures or non-racemic mixtures of stereoisomers are well known in the art. For example, optically active (R)- and (S)-isomers may be prepared using chiral synthons or chiral reagents, or resolved using conventional techniques. Enantiomers (R- and S-isomers) may be resolved by methods known to one of ordinary skill in the art, for example by: formation of diastereoisomeric salts or complexes which may be separated, for example, by crystallization; via formation of diastereoisomeric derivatives which may be separated, for example, by crystallization, selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where a desired enantiomer is converted into another chemical entity by one of the separation procedures described above, a further step may be required to liberate the desired enantiomeric form. Alternatively, a specific enantiomer may be synthesized by asymmetric synthesis using optically active reagents, substrates, catalysts or solvents or by converting an enantiomer to the other by asymmetric transformation. For a mixture of enantiomers, enriched in a particular enantiomer, the major component enantiomer may be further enriched (with concomitant loss in yield) by recrystallization.

In addition, the compounds of the present invention can exist in unsolvated as well as solvated forms with pharmaceutically acceptable solvents such as water, ethanol, and the like. In general, a depiction of the compound by structure or name is considered to embrace the compound in any form (e.g., by itself, as a solvate, or otherwise in a mixture).

The chemistry for the preparation of the compounds of this invention is known to those skilled in the art. In fact, there may be more than one process to prepare the compounds of the invention. The following examples illustrate but do not limit the invention. All references cited herein are incorporated by reference in their entirety.

An Intermediate of formula (c) where R¹, R², R³, and R⁴ are as defined in the Summary of the Invention for a Compound of Formula I can be prepared according to Scheme 1.

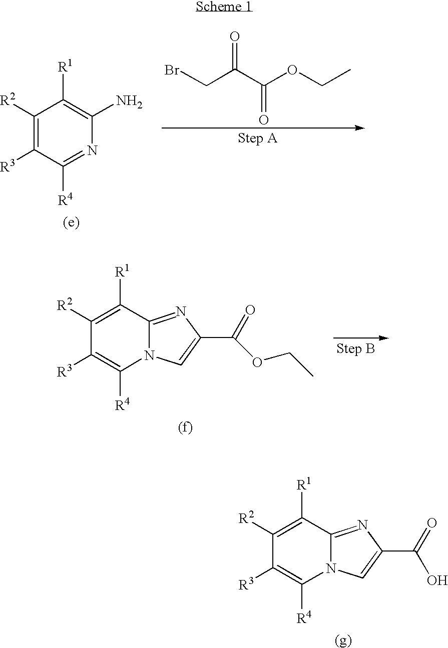

The reaction in Step A is carried out in the presence of a solvent such as ethanol and with heat at a temperature of about 80° C. The reaction in Step B is carried out in the presence of a base such as NaOH in a solvent such as methanol and/or water and at a temperature of about 50° C.

An Intermediate of formula (i) where R⁶, R⁷, and R⁸ are as defined in the Summary of the Invention for a Compound of Formula I can be prepared according to Scheme 2.

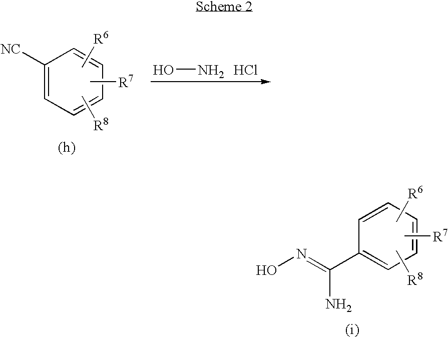

The reaction is carried out in the presence of a solvent such as ethanol, in the presence of a base such as triethylamine, and at a temperature of about 85° C.

A compound of the invention of Formula I(a) where R¹, R², R³, R⁴, and R⁵ are as defined in the Summary of the Invention for a Compound of Formula I can be prepared according to Scheme 3.

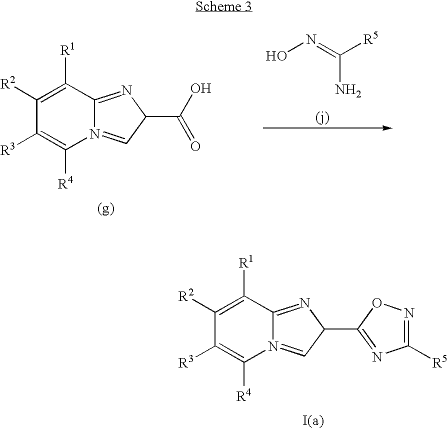

The reaction is carried out with a coupling agent such as EDCI in the presence of HOBt and carried out at room temperature in a solvent such as DMF. Following completion of the coupling reaction, generally within one hour, the reaction is heated to a temperature of about 100° C. for approximately overnight to yield a Compound of the Invention of Formula I(a).

A compound of the invention of Formula I(j) where R¹, R², R³, R⁴, and R⁵ are as defined in the Summary of the Invention for a Compound of Formula I can be prepared according to Scheme 4.

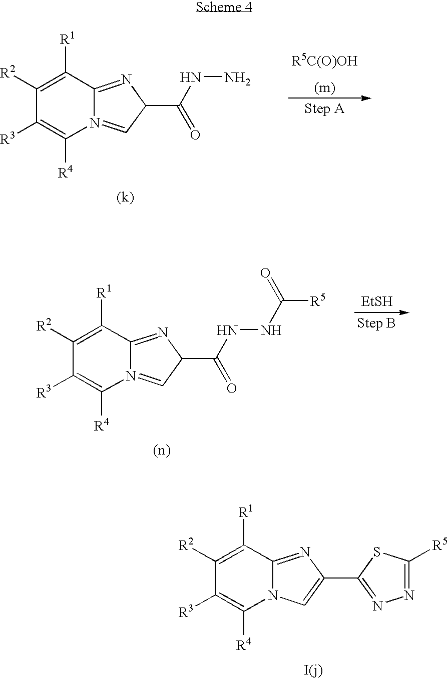

The reaction is carried out with a coupling agent such as EDCI in the presence of HOBt and carried out at room temperature in a solvent such as DMF. Following completion of the coupling reaction, generally within one hour, the reaction is heated to a temperature of about 100° C. for approximately overnight. The reaction in Step B is carried out in a solvent such as toluene in the presence of Lawesson's reagent, a base such as pyridine, and P₂S₅ at about reflux to yield a Compound of the Invention of Formula I(j).

A compound of the invention of Formula I(e) where R¹, R², R³, R⁴, and R⁵ are as defined in the Summary of the Invention for a Compound of Formula I can be prepared according to Scheme 5.

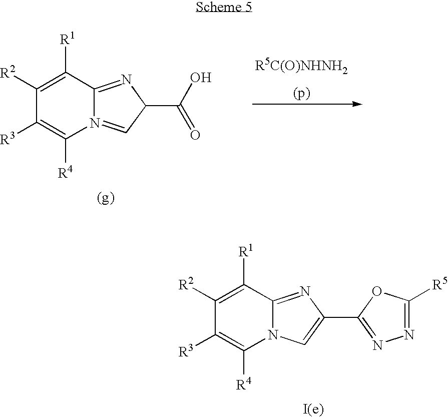

The reaction is carried out in a solvent such as DCM and in the presence of imidazolinium chloride and a base such as triethylamine is added slowly at a temperature of about 0° C. The reaction is allowed to warm to room temperature and proceed until completion.

A compound of the invention of Formula I(c) where R¹, R², R³, R⁴, and R⁵ are as defined in the Summary of the Invention for a Compound of Formula I can be prepared according to Scheme 6.

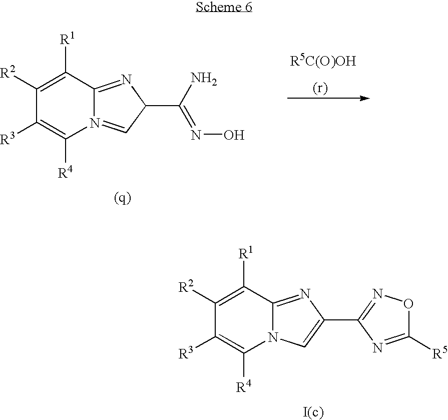

The reaction is treated with a coupling agent such as EDCI in the presence of HOBt and carried out at room temperature in a solvent such as DMF. Following completion of the coupling reaction, generally within one hour, the reaction is heated to a temperature of about 100° C. for approximately overnight to yield a Compound of Formula I(c).

A compound of the invention of Formula I(p) where R¹³ is alkyl substituted with one or two groups independently selected from halo, hydroxy, alkoxy, —C(O)OR¹⁰, —OC(O)R^{10b}, —C(O)R^{10b}, and —NR¹¹R^{11a} or R¹³ is heterocycloalkyl optionally substituted with 1 or 2 groups independently selected from alkyl, carboxy, hydroxyalkyl, and carboxyalkyl, and where R¹, R², R³, R⁴, the A ring, R⁷, and R⁸ and all other groups are as defined in the Summary of the Invention for a Compound of Formula I can be prepared according to Scheme 7.

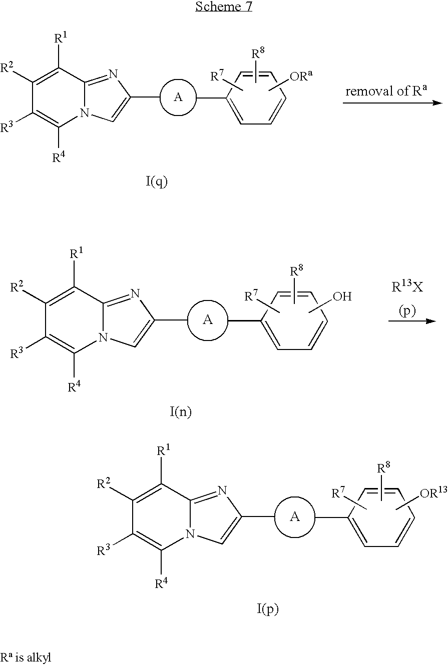

When R^{a} is methyl, it can be removed in the presence of AlCl₃ and EtSH, in a solvent such as DCM. Alternatively, when R^{a} is methyl, it can be removed in the presence of BBR₃ in a solvent such as toluene. The Compound of Formula I(n) is then treated with a reagent of formula R¹³X where X is halo and R¹³ is as defined above, in the presence of a base such as K₂CO₃ in a solvent such as DMF or in the presence of a base such as NaOH in a solvent such as ethanol to yield a Compound of Formula I(p).

A Compound of the invention of Formula I(r) where R⁵ is phenyl substituted with R⁶, R⁷, and R⁶ is as defined in Table 3 and R⁸ and R⁷, R⁸, and all other groups are as defined in the Summary of the Invention can be prepared from other Compounds of Formula I. For example, see Table 3 for representative conditions.

TABLE 3

| | |
|---|---|
| | I(r) |
| 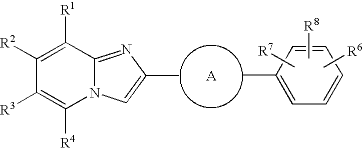 | |

| R⁶, Starting Material | Conditions | R⁶, Final Compound |
|---|---|---|
| alkyl substituted with at least | R¹⁰ is t-Bu: TFA, a solvent | alkyl substituted with at least |
| one R⁹ where one of the R⁹ is | such as DCM | one R⁹ where one of the R⁹ is |
| —C(O)OR¹⁰ and R¹⁰ is alkyl | R¹⁰ is Me, Et: LiOH, a solvent | —C(O)OR¹⁰ and R¹⁰ is |
| | such as H₂O, H₂O/THF | hydrogen |
| alkyl substituted with at least | R^{12a}S(O)₂Cl, base such as | alkyl substituted with at least |
| one R⁹ where one of the R⁹ is | Et₃N, a solvent such as DCM | one R⁹ where one of the R⁹ is |
| NR¹¹R^{11a} and R¹¹ and R^{11a} | | —NR¹²S(O)₂R^{12a} |
| are hydrogen | | |
| alkyl substituted with at least one R⁹ where one of the R⁹ is —C(O)H | NHR¹¹R^{11a} or 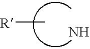 (where R′ is hydroxy, carboxy, alkoxycarbonyl, alkyl, hydroxyalkyl or alkoxycarbonylamino), NaCNBH₄, a solvent such as acetic acid/MeOH | alkyl substituted with at least one R⁹ where one of the R⁹ is NR¹¹R^{11a} or R⁹ is heterocycloalkyl optionally substituted with groups independently selected from hydroxy, carboxy, alkoxycarbonyl, alkyl, hydroxyalkyl, and alkoxycarbonylamino |
| alkyl substituted with 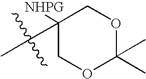 where PG is a N-protecting group | when PG is BOC: TFA in a solvent such as DCM | alkyl substituted with 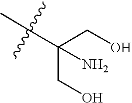 |
| cycloalkyl substituted with | R¹⁰ is t-Bu: TFA, a solvent | cycloalkyl substituted with |
| one or two alkoxycarbonyl | such as DCM | one or two carboxy |
| cycloalkyl substituted with | NHR¹⁰R^{10a}, amide formation | cycloalkyl substituted with 1 |
| one or two carboxy | conditions | or 2 —C(O)NR¹⁰R^{10a} and R¹⁰ |
| | | and R^{10a} are as defined in the |
| | | Summary of the Inv. for a |
| | | Cmpd of Form. I |
| alkenyl | OsO₄, NMO, a solvent such | alkyl substituted with 2 R⁹ |
| | as acetone/H₂O | and the R⁹ are hydroxy |
| —NR¹¹R^{11a} and R¹¹ and R^{11 a} | R^{12a}S(O)₂Cl, pyridine | —NR¹²S(O)₂R^{12a} and R¹² is |
| are hydrogen | | hydrogen and R^{12a} is as |
| | | defined in the Summary of |
| | | the Inv. for a Cmpd of Form. I |
| —NR¹¹R^{11a} and R¹¹ and R^{11a} | R^{11a}Cl, K₂CO₃, a solvent | —NR¹¹R^{11a} and R¹¹ and R^{11a} is |
| are hydrogen | such as DMF | alkyl, alkenyl, alkynyl, |
| | | carboxyalkyl, or hydroxyalkyl |
| —C(O)H | NHR¹¹R^{11a}, NaCNBH₄, a | —CH₂R₉ where R₉ is |
| | solvent such as acetic | —NR¹¹R^{11a} and R¹¹ is |
| | acid/MeOH | hydrogen and R^{11a} is as |
| | | defined in the Summary of |
| | | the Inv. for a Cmpd of Form. I |
| —C(O)H | 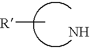 (where R′ is hydroxy, carboxy, or hydroxyalkyl), NaCNBH₄, a solvent such as acetic acid/MeOH | —CH₂R⁹ where R⁹ heterocycloalkyl optionally substituted with 1 or 2 groups independently selected from hydroxy, carboxy, and hydroxyalkyl |
| —C(O)H | H₂NS(O)₂R^{12a}, NaCNBH₄, a | —CH₂R⁹ where R⁹ is |
| | solvent such as acetic | —NR¹²S(O)₂R^{12a} where R¹² is |
| | acid/MeOH | hydrogen and R^{12a} is as |
| | | defined in the Summary of |
| | | the Inv. for a Cmpd of Form. I |
| —OR¹³ where R¹³ is alkenyl | OsO₄, NMO, a solvent such | —OR¹³ where R¹³ is alkyl |
| | as acetone/H₂O | substituted with 2 hydroxy |
| —OR¹³ where R¹³ is alkyl | DIEA, MsCl, a solvent such | —OR¹³ where R¹³ is alkyl |
| substituted with 2 hydroxy | as THF then NH₃, a solvent | substituted with 1 hydroxy |
| | such as MeOH | and 1 NH₂ |
| —OR¹³ where R¹³ is alkyl | R¹⁰ is t-Bu: TFA a solvent | —OR¹³ where R¹³ is alkyl |
| substituted with —C(O)OR¹⁰ | such as DCM | substituted with —C(O)OR¹⁰ |
| where R¹⁰ is alkyl | R¹⁰ is Et: LiOH a solvent | where R¹⁰ is hydrogen |
| | such as H₂O, H₂O/THF | |
| —OR¹³ where R¹³ is alkyl | NHR¹¹R^{11a}, NaCNBH₄, a | —OR¹³ where R¹³ is alkyl |
| substituted with —C(O)H or | solvent such as acetic | substituted with —NR¹¹R^{11a} |
| —C(O)R^{10b} where R^{10b} is | acid/MeOH | |
| alkyl | | |
| —OR¹³ where R¹³ is 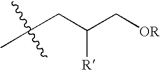 where 1) R is an OPG where PG¹ is an O-protecting group and R′ is OH; or 2) R is H and R′ is NHPG² where PG² is a N-protecting group | 1) when PG¹ is TBDMS: phosphoramidite, tetrazole, H₂O₂, Na₂S₂O₃ in a solvent such as DCM to yield 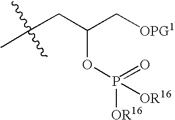 where each R¹⁶ is alkyl, followed by treatment with an acid such as HCl in a solvent such as ethanol; 2) when PG² is BOC: phosphoramidite, tetrazole, in a solvent such as DCM to yield 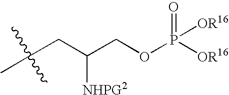 each R¹⁶ is alkyl, followed by treatment with an acid such as HCl in a solvent such as ethanol | 1) —OR¹³ where R¹³ is 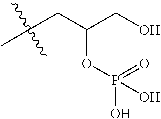 2) —OR¹³ where R¹³ is 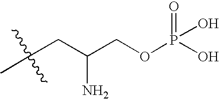 |
| —OR¹³ where R¹³ is 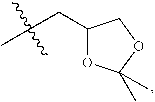 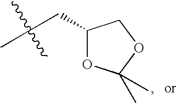 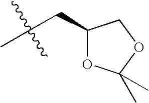 | acid such as 1H HCl, solvent such as THF | —OR¹³ where R¹³ is 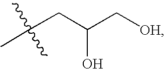 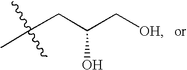 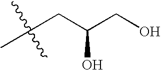 |
| —OR¹³ where R¹³ is 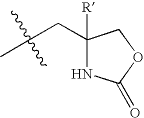 where R′ is H or —CH₃ | a base such as Ba(OH)₂ in solvents such as EtOH/H₂O followed by treatment with an acid such as HCl in a solvent such as ethanol | —OR¹³ where R¹³ is 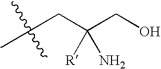 |
| hydroxy | 1) R¹⁶ is alkyl: 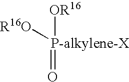 X is halo base such as K₂CO₃, a solvent such as DMF 2) R¹⁶ is H: treat the product from 1) with TMSBr in a solvent such as DCM | —OR¹³ where R¹³ is alkyl substituted with —P(O)(OR¹⁶)₂ |
| hydroxy | 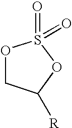 R is H or alkyl base such as K₂CO₃, Bu₄NHS(O)₄, a solvent such as THF | —OR¹³ where R¹³ is alkyl substituted with —OS(O)₂OH or a salt thereof |
| hydroxy | 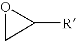 (where R′ is R,S-CH₃, R-CH₃, S-CH₃ , R,S-CF₃, R-CF₃, S-CF₃) 1) base such as NaOH, solvent such as THF or 2) LiCl, Et₃N in a solvent such as ethylene glycol | —OR¹³ where R¹³ is alkyl substituted with one hydroxy or R¹³ is alkyl substituted with one hydroxy and three fluoro |

### Synthetic Examples

### Intermediate 10

### 8-Chloro-6-(trifluoromethyl)imidazo[1,2-a]pyridine-2-carboxylic acid

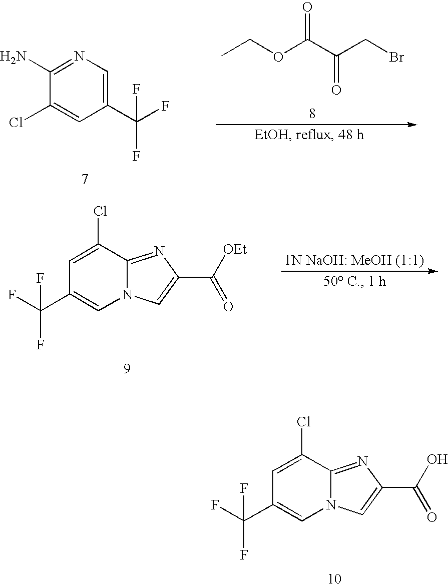

Ethyl 8-chloro-6-(trifluoromethyl)-imidazo[1,2-a]pyridine-2-carboxylate (9). To a stirred solution of 2-amino-3-chloro-5-trifluoromethyl pyridine 7 (12.5 g, 63.6 mmol) in EtOH (125 mL) was added ethyl bromopyruvate 8 (20 mL, 159 mmol) at room temperature. The resulting mixture was heated to 80° C. for 12 h. The reaction mixture was cooled to ambient temperature and concentrated. The residue was suspended in diethyl ether and the resulting solid was filtered and dried under vacuum to afford 9 (17.3 g, 93% yield) as a light yellow solid.

8-Chloro-6-(trifluoromethyl)-imidazo[1,2-a]pyridine-2-carboxylic acid (10). To a stirred solution of ester 9 (10 g, 34 mmoL) in MeOH (100 mL) was added 1 M NaOH (100 mL). The mixture was heated to 50° C. for 1 h. The reaction mixture was concentrated in vacuo. Water was added to the residue and the mixture acidified to pH 4 using acetic acid. The resulting precipitate was filtered, washed with water, and dried to afford intermediate 10 (7.2 g, 80% yield) as a white solid.

### Intermediate 150

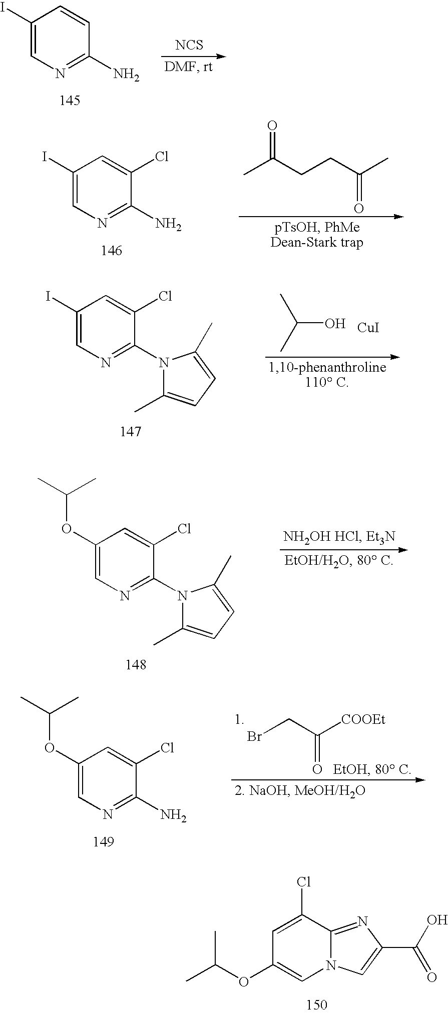

2-Amino-3-chloro-5-iodopyridine (146). To a solution of 2-amino-5-iodopyridine 145 (10 g, 46 mmol) in DMF (40 mL) was added N-chlorosuccinimide (6.6 g, 50 mmol). The stirring was continued for 12 h at room temperature. The mixture was poured into water (200 mL) and extracted with DCM. The extracts were dried over Na₂SO₄ and concentrated under reduced pressure. The residue was purified by column chromatography to give 2-amino-3-chloro-5-iodopyridine 146 (4.5 g, 38% yield). ¹H-NMR (400 MHz, DMSO-d₆) δ 8.06 (d, 1H), 7.87 (d, 1H), 6.52 (br s, 2H).

3-Chloro-2-(2,5-dimethyl-1H-pyrrol-1-yl)-5-iodopyridine (147). A mixture of 2-amino-3-chloro-5-iodopyridine 146 (6.2 g, 24 mmol), 2,5-hexadione (3.3 g, 29 mmol) and p-toluenesulfonic acid monohydrate (456 mg, 2.40 mmol) in toluene (50 mL) was heated to reflux with a Dean-Stark trap for 5 h. The mixture was cooled to room temperature and washed with saturated sodium bicarbonate solution. The organic phase was dried over Na₂SO₄ and concentrated under reduced pressure to give crude 147 which was used in the next step without further purification. ¹H-NMR (400 MHz, DMSO-d₆) δ 8.84 (d, 1H), 8.70 (d, 1H), 5.82 (s, 2H), 1.90 (s, 6H).

3-Chloro-2-(2,5-dimethyl-1H-pyrrol-1-yl)-5-isopropoxypyridine (148). A mixture of 3-chloro-2-(2,5-dimethyl-1H-pyrrol-1-yl)-5-iodopyridine 147 (2 g, 6 mmol), CuI (114 mg, 0.599 mmol), Cs₂CO₃ (3.91 g, 12.0 mmol), and 1,10-phenanthroline (250 mg, 1.2 mmol) in iPrOH (25 mL) was heated to 110° C. in a sealed tube. The stirring was continued for 12 h. Removal of the solvent and further purification by column chromatography gave 3-chloro-2-(2,5-dimethyl-1H-pyrrol-1-yl)-5-isopropoxypyridine 148 (820 mg, 51% yield). MS (EI) for C₁₄H₁₇ClN₂O, found 265.1 (MH+).

3-Chloro-5-isopropoxypyridin-2-amine (149). To a mixture of 3-chloro-2-(2,5-dimethyl-1H-pyrrol-1-yl)-5-isopropoxypyridine 148 (820 mg, 3.1 mmol) and hydroxylamine hydrochloride (6.5 g, 93 mmol) in EtOH (10 mL) and water (3 mL) was added triethylamine (2.1 mL, 15 mmol). The mixture was heated to 80° C. and the stirring was continued for 20 h. After cooling to RT, the reaction was acidified with 1 N HCl and extracted with ethyl ether. The aqueous phase was basified to pH 9, and extracted with DCM. The solution was dried over Na₂SO₄ and filtered. Removal of DCM gave crude 149, which was used in the next step without purification. MS (EI) for C₈H₁₁ClN₂O, found 187.1 (MH+).

8-Chloro-6-isopropoxyimidazo[1,2-a]pyridine-2-carboxylic acid (150). To a solution of 3-chloro-5-isopropoxypyridin-2-amine 149 (576 mg, 3.09 mmol) in EtOH (15 mL) was added ethyl bromopyruvate (0.6 mL, 4 mmol). The mixture was stirred at 80° C. for 12 h. EtOH was removed under reduced pressure. The residue was dissolved in MeOH (8 mL) and water (8 mL) and treated with NaOH (372 mg, 9.30 mmol) at 60° C. for 3 h. The solution was then concentrated and the pH adjusted to 3 with 1 N HCl. Filtration of the mixture gave 8-chloro-6-isopropoxyimidazo[1,2-a]pyridine-2-carboxylic acid 150 (340 mg, 43% over two steps). ¹H-NMR (400 MHz, DMSO-d₆) δ 12.80 (br s, 1H), 8.43 (s, 1H), 8.32 (d, 1H), 7.43 (d, 1H), 4.51 (sep, 1H), 1.31 (d, 6H); MS (EI) for C₁₁H₁₁ClN₂O₃, found 255.1 (MH+).

### Intermediate 152

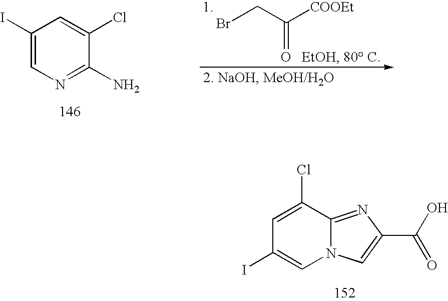

3-chloro-5-iodopyridin-2-amine (146). Intermediate 146 was prepared from Intermediate 145 whose synthesis is described in Intermediate 150.

8-Chloro-6-iodoimidazo[1,2-a]pyridine-2-carboxylic acid (152). To a solution of 3-chloro-5-iodopyridin-2-amine 146 (4.0 g, 16 mmol) in EtOH (100 mL) was added ethyl bromopyruvate (2.5 mL, 19 mmol). The mixture was stirred at 80° C. for 12 h. EtOH was removed under reduced pressure. The residue was dissolved in MeOH (25 mL) and water (25 mL) and treated with NaOH (1.88 g, 47.0 mmol) at 60° C. for 3 h. The solution was then concentrated and the pH adjust to 3 with 1 N HCl. Filtration of the mixture gave 8-chloro-6-iodoimidazo[1,2-a]pyridine-2-carboxylic acid 152 (2.6 g, 51% over two steps from intermediate 145, see Intermediate 150, Step 1). ¹H-NMR (400 MHz, DMSO-d₆) M3.03 (br s, 1H), 8.93 (d, 1H), 8.46 (s, 1H), 7.81 (d, 1H); MS (EI) for C₈H₄ClIN₂O₂, found 323.1 (MH+).

### Intermediate 83

### 8-Chloro-N′-hydroxy-6-(trifluoromethyl)imidazo[1,2-a]pyridine-2-carboximidamide

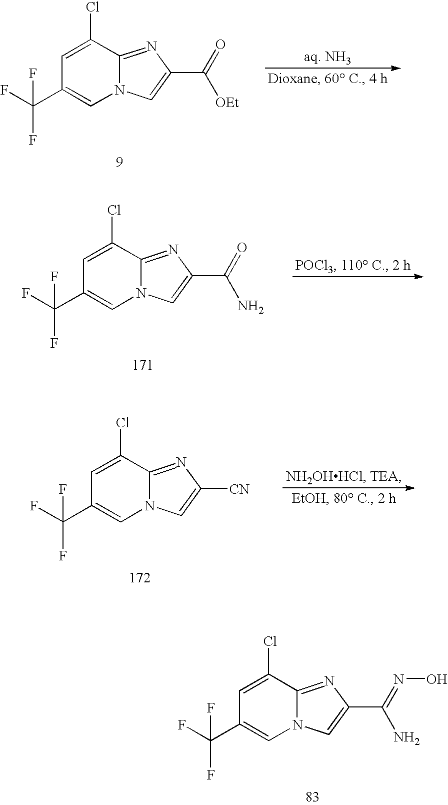

8-Chloro-6-(trifluoromethyl)imidazo[1,2-a]pyridine-2-carboxamide (171). To a stirred solution of 9 (5.0 g, 17.12 mmol), prepared as described in Intermediate 10, in dioxane (30 mL) was added NH₄OH (60 mL) and the reaction was stirred at 60° C. for 4 h in a sealed tube. Solvent was removed and the residue obtained was crystallize from EtOAc, filtered and dried to obtain 171 (4 g, 88.88%).

8-Chloro-6-(trifluoromethyl)imidazo[1,2-a]pyridine-2-carbonitrile (172). A stirred solution of 171 (4.0 g, 15.2 mmol) in POCl₃ (32 mL) was heated at 110° C. for 2 h. Later, solvent was removed from the reaction and a cold solution of NaHCO₃ was added for neutralization. The aqueous layer was extracted with EtOAc. The organic layer was dried over Na₂SO₄, filtered and concentrated to obtain 172 (3.0 g, 81.08%) which was used for the next step without further purification.

8-Chloro-N′-hydroxy-6-(trifluoromethyl)imidazo[1,2-a]pyridine-2-carboximidamide (83). A solution of NH₂OH.HCl (3.0 g, 43.47 mmol) and Et₃N (12 mL, 86.2 mmol) in EtOH (15 mL) was stirred at rt for 30 min. To this was added 172 (3 g, 12.24 mmol) and the reaction mixture was heated at 80° C. for 2 h. Solvent was then removed from the reaction mixture. Water was added and the aqueous layer was extracted with EtOAc. The organic layer was dried over Na₂SO₄, filtered and concentrated to obtain 83 (1.8 g, 52.94%) which was used in subsequent steps without further purification.

### Example 1

### 3-(3-Chloro-4-(5-(8-chloro-6-(trifluoromethyl)-imidazo[1,2-a]pyridin-2-yl)-1,2,4-oxadiazol-3-yl)phenyl)propanoic acid

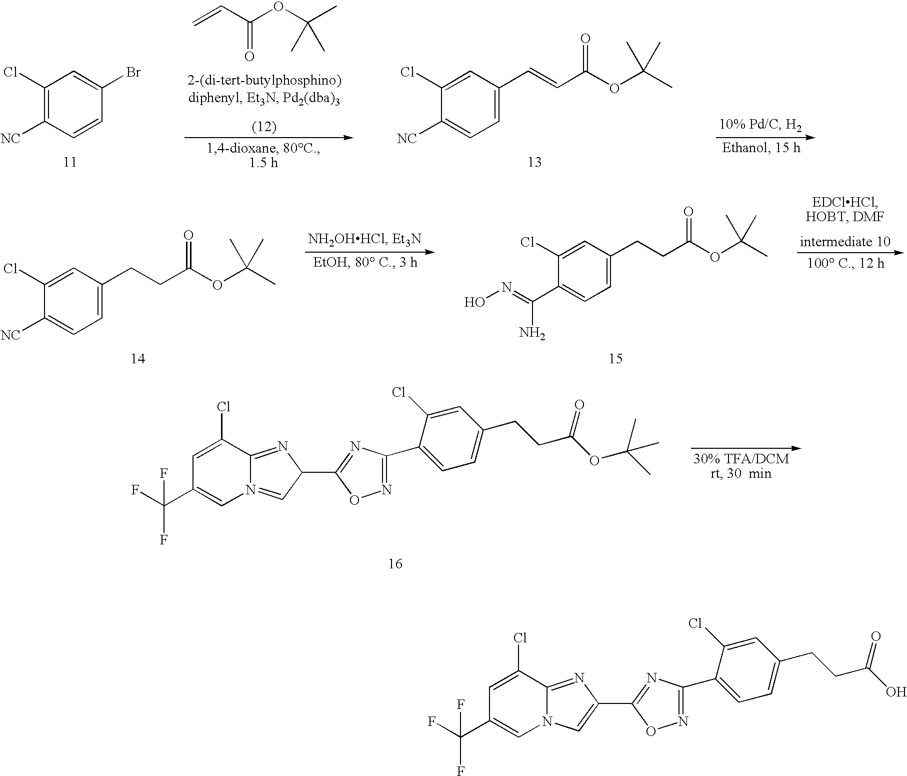

tert-Butyl 3-(3-chloro-4-cyanophenyl)acrylate (13).To a stirred solution of 4-bromo-2-chlorobenzonitrile 11 (2.0 g, 9.3 mmol) in 1,4-dioxane (25 mL) was added tent-butyl acrylate 12 (1.7 g, 14 mmol). The resulting mixture was purged with argon gas. To this solution, Pd₂(dba)₃ (47 mg, 0.05 mmol) and (2-biphenyl)di-tert-butylphosphine (13 mg, 0.05 mmol) were added and again purged with argon followed by addition of N,N,N-triethylamine (1.86 g, 18.5 mmol). The reaction mixture was stirred at room temperature for 20 min, then heated to 80° C. for 1.5 h. The reaction mixture was concentrated in vacuo, then diluted with EtOAc and filtered. The filtrate was washed with water and saturated NaCl. The organic layers were combined, dried over Na₂SO₄, and concentrated in vacuo to afford 13 (2.2 g, 90% yield) as a yellowish solid. ¹H NMR (400 MHz, DMSO-d₆) δ 8.2 (s, 1H), 8.0 (d, 1H), 7.9 (d, 1H), 7.6 (d, 1H), 6.8 (d, 1H), 1.5 (s, 9H).

tert-Butyl 3-(3-chloro-4-cyanophenyl)propanoate (14). To a stirred solution of 13 (2.2 g, 8.3 mmol) in EtOH (75 mL) was added 10% Pd/C (200 mg). The reaction mixture was stirred overnight at room temperature under a hydrogen balloon. The reaction mixture was filtered and the filtrate was concentrated in vacuo to afford 2.3 g of 14 as a greenish yellow semi solid. ¹H NMR (400 MHz, CDCl₃) δ 7.6 (d, 1H), 7.4 (s, 1H), 7.2 (d, 1H), 2.9 (t, 2H), 2.5 (t, 2H), 1.4 (s, 9H).

tert-Butyl 3-(4-amidino-3-chlorophenyl)propanoate (15). To a stirred solution of hydroxylamine hydrochloride (3.56 g, 51.6 mmol) in EtOH (20 mL) was added N,N,N-triethylamine (6.1 g, 60 mmol). After stirring for 30 min at room temperature, intermediate 14 (2.3 g, 8.6 mmol) in EtOH (25 mL) was added. The resulting mixture was then stirred at 80° C. for 3 h. The reaction mixture was concentrated in vacuo and the resulting residue was dissolved in EtOAc, washed with water (2×), saturated NaCl. The organic layers were combined, dried over Na₂SO₄, and concentrated to afford hydroxyimidate 15 (2.3 g, 90% yield) as a yellowish solid.

tert-Butyl 3-(3-chloro-4-(5-(8-chloro-6-(trifluoromethyl)-imidazo[1,2-a]pyridin-2-yl)-1,2,4-oxadiazol-3-yl)phenyl)propanoate (16). To a stirred solution of hydroxyimidate 15 (1.4 g, 4.7 mmol) in DMF (15 mL) was added intermediate 10 (1.8 g, 7.1 mmol), EDCI.HCl (1.3 g, 7.1 mmol) and HOBT (0.94 g, 7.1 mmol). The mixture was stirred at room temperature for 1 h, then heated to 100° C. for 15 h. The reaction mixture was concentrated in vacuo. The residue was purified by column chromatography (15% EtOAc/hexane) to afford 16 (0.8 g, 33% yield) as a yellow oil. ¹H NMR (400 MHz, CDCl₃) δ 8.6 (d, 2H), 8.0 (d, 1H), 7.6 (s, 1H), 7.4 (s, 1H), 7.3 (s, 1H), 3.0 (t, 2H), 2.6 (t, 2H), 1.4 (s, 9H).

3-(3-Chloro-4-(5-(8-chloro-6-(trifluoromethyl)-imidazo[1,2-a]pyridin-2-yl)-1,2,4-oxadiazol-3-yl)phenyl)propanoic acid. A solution of 16 (0.6 g, 1.1 mmol) in 30% TFA/DCM (10 mL) was stirred for 30 min. The reaction mixture was concentrated in vacuo. The residue was triturated with diethyl ether, and the ether layer decanted. To the residue was added iPrOH and the mixture was stirred. The resulting precipitate was filtered and dried under vacuum to afford the product (0.28 g, 52% yield) as a white solid. ¹H NMR (400 MHz, DMSO-d₆) δ 12.2 (s, 1H), 9.3 (s, 1H), 9.05 (s, 1H), 8.05 (s, 1H), 7.95 (d, 1H), 7.6 (s, 1H), 7.45 (d, 1H), 2.9 (t, 2H), 2.65 (t, 2H); MS (EI) for C₁₉H₁₁Cl₂F₃N₄O₃, found 471 (MH+).

The following compounds were prepared using the same or analogous synthetic techniques in Example 1 and substituting with appropriate reagents, which were commercially available or were prepared using procedures herein or procedures known to one of ordinary skill in the art.

3-(4-{5-[8-Chloro-6-(trifluoromethyl)imidazo[1,2-a]pyridin-2-yl]-1,2,4-oxadiazol-3-yl}-3-methylphenyl)propanoic acid. ¹H-NMR (400 MHz, DMSO-d₆) δ 12.20 (br s, 1H), 9.32 (s, 1H), 9.04 (s, 1H) 8.06 (s, 1H), 7.96 (m, 1H), 7.32 (s, 1H), 7.28 (m, 1H), 2.89 (t, 2H), 2.64 (t, 2H), 2.60 (s, 3H).

3-(4-{5-[8-Chloro-6-(trifluoromethyl)imidazo[1,2-a]pyridin-2-yl]-1,2,4-oxadiazol-3-yl}-2-fluorophenyl)propanoic acid. MS (EI) for C₁₉H₁₁ClF₄N₄O₃, found 455 (MH+).

3-[4-{5-[8-Chloro-6-(trifluoromethyl)imidazo[1,2-a]pyridin-2-yl]-1,2,4-oxadiazol-3-yl}-3-(trifluoromethyl)phenyl]propanoic acid. MS (EI) for C₂₀H₁₁ClF₆N₄O₃, found 505 (MH+).

3-(4-{5-[8-Chloro-6-(trifluoromethyl)imidazo[1,2-a]pyridin-2-yl]-1,2,4-oxadiazol-3-yl}-3,5-difluorophenyl)propanoic acid. MS (EI) for C₁₉H₁₀ClF₅N₄O₃, found 473 (MH+).

3-(4-{5-[8-Chloro-6-(trifluoromethyl)imidazo[1,2-a]pyridin-2-yl]-1,2,4-oxadiazol-3-yl}-3-fluorophenyl)propanoic acid. MS (EI) for C₁₉H₁₁ClF₄N₄O₃, found 455 (MH+).

3-(3-Chloro-4-{5-[8-chloro-6-(trifluoromethyl)imidazo[1,2-a]pyridin-2-yl]-1,2,4-oxadiazol-3-yl}phenyl)propanamide (prepared by treating the product of Example 1 with NH₃ using conditions known to one of skill in the art). MS (EI) for C₁₉H₁₂Cl₂F₃N₅O₂, found 470 (MH+).

3-(4-{5-[8-Chloro-6-(trifluoromethyl)imidazo[1,2-a]pyridin-2-yl]-1,2,4-oxadiazol-3-yl}phenyl)propanoic acid. ¹H-NMR (400 MHz, DMSO-d₆) δ 12.18(br s, 1H), 9.35 (s, 1H), 9.03 (s, 1H), 8.02 (m, 3H), 7.45 (m, 2H), 2.92 (t, 2H), 2.59 (t, 2H); MS (EI) for C₁₉H₁₂ClF₃N₄O₃, found 437 (MH+).

3-(4-{5-[8-Chloro-6-(trifluoromethyl)imidazo[1,2-a]pyridin-2-yl]-1,2,4-oxadiazol-3-yl}-2-methylphenyl)propanoic acid. MS (EI) for C₂₀H₁₄ClF₃N₄O₃, found 451 (MH+).

3-(2-Chloro-4-{5-[8-chloro-6-(trifluoromethyl)imidazo[1,2-a]pyridin-2-yl]-1,2,4-oxadiazol-3-yl}phenyl)propanoic acid. MS (EI) for C₁₉H₁₁Cl₂F₃N₄O₃, found 471 (MH+).

3-(2,6-Dichloro-4-{5-[8-chloro-6-(trifluoromethyl)imidazo[1,2-a]pyridin-2-yl]-1,2,4-oxadiazol-3-yl}phenyl)propanoic acid. MS (EI) for C₁₉H₁₀Cl₃F₃N₄O₃, found 507 (MH+).

8-chloro-2-(3-{2-chloro-4-[2-(methylsulfonyl)ethyl]phenyl}-1,2,4-oxadiazol-5-yl)-6-(trifluoromethyl)imidazo[1,2-a]pyridine. ¹H-NMR (400 MHz, DMSO-d₆) δ 9.32 (s, 1H), 9.04 (s, 1H), 8.05 (s, 1H), 7.97 (d, 1H), 7.71 (s, 1H), 7.52 (d, 1H), 3.14 (d, 2H), 3.01 (s, 2H); MS (EI) for C₁₉H₁₃Cl₂F₃N₄O₃S, found 505 (MH+).

3-(2-chloro-4-{5-[8-chloro-6-(trifluoromethyl)imidazo[1,2-a]pyridin-2-yl]-1,2,4-oxadiazol-3-yl}-6-fluorophenyl)propanoic acid. MS (EI) for C₁₉H₁₀C₁₂F₄N₄O₃, found 488.9 (MH+).

### Example 2

### tert-Butyl 3-(5-chloro-2-fluoro-4-(5-(6-(trifluoromethyl)imidazo[1,2-a]pyridin-2-yl)-1,2,4-oxadiazol-3-yl)phenyl)propanoate

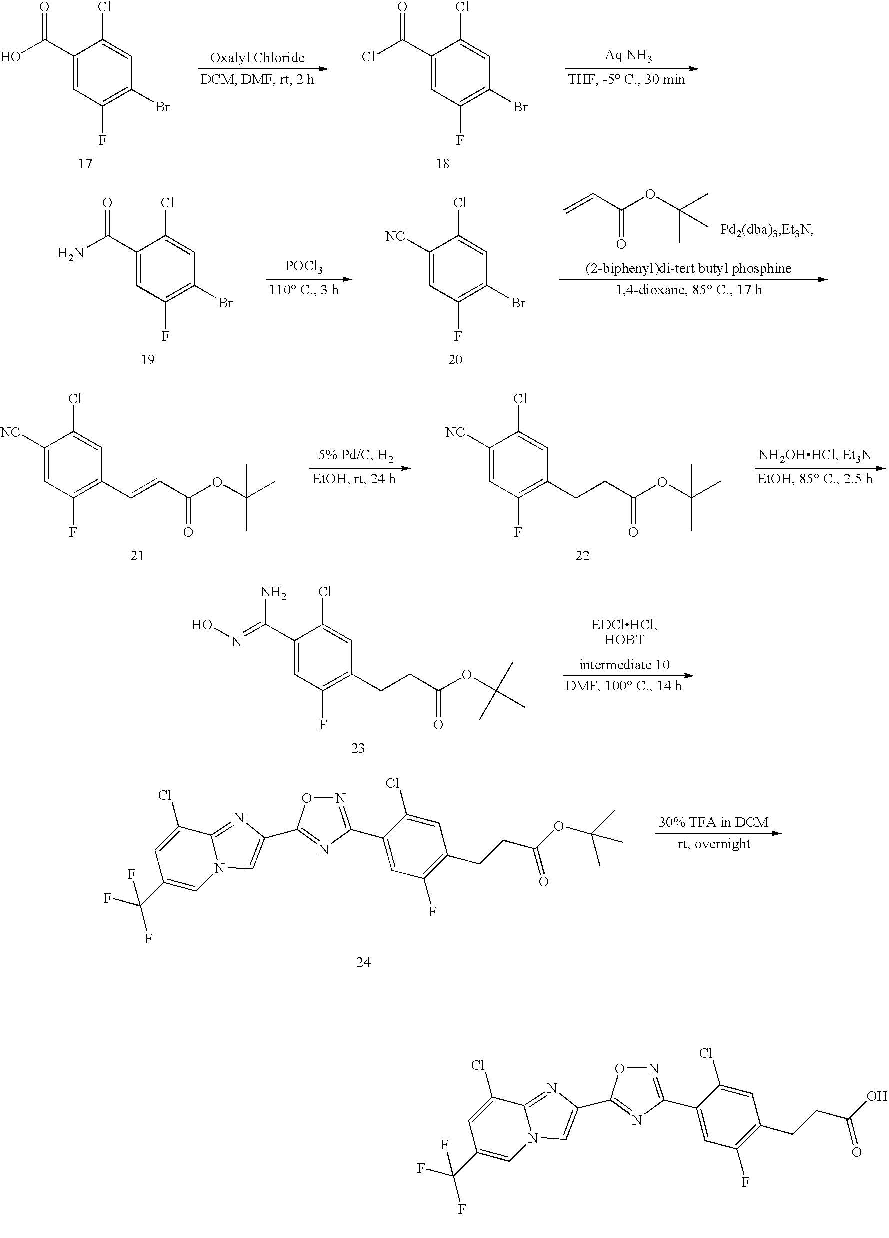

2-Chloro-4-bromo-5-fluoro benzamide (19). To a stirred solution of 17 (20.0 g, 79.0 mmol) in DCM (200 mL) with a catalytic amount of DMF, was added oxalyl chloride (37.5 g, 295 mmol) at 0° C. After the addition was complete, the reaction mixture was stirred at 0° C. for 15 min, followed by stirring at room temperature for 2 h. The reaction mixture was concentrated in vacuo to afford 18 as a white solid. The acid chloride 18 was dissolved in THF (300 mL) and was added to aq. NH₃ (600 mL) at −5° C. and stirred for 30 min. The resulting reaction mixture was concentrated in vacuo. The precipitated solid was filtered and washed successively with water and hexane to afford 19 (15 g, 81% yield) as a white solid.

2-Chloro-4-bromo-5-fluoro benzonitrile (20). A solution of 19 (18.0 g, 71.0 mmol) in POCl₃ (100 mL) was stirred at room temperature, followed by heating to 110° C. for 3 h. After completion of the reaction, the POCl₃ was removed in vacuo and the residue was dissolved in a minimum amount of water and extracted into DCM. The organic layer was washed with saturated sodium bicarbonate solution and water. The organic layers were combined, dried over Na₂SO₄, and concentrated in vacuo to afford 20 (15.6 g, 93.0% yield) as an off-white solid.

tert-Butyl 3-(5-chloro-4-cyano-2-fluorophenyl)acrylate (21). To a stirred solution of 20 (15.6 g, 66.5 mmol) in 1,4-dioxane (100 mL) was added tent-butyl acrylate (7.72 g, 60.2 mmol) and N,N,N-triethylamine (16.8 g, 167 mmol) at room temperature. The reaction mixture was then degassed with argon for 15 minutes. (2-Biphenyl)di-tert-butyl phosphine (300 mg, 1 mmol) and Pd₂(dba)₃ (510 mg, 0.50 mmol) were added and the reaction mixture was again degassed with argon for 15 min. The reaction mixture was then heated to 85° C. for 17 h. The reaction mixture was concentrated in vacuo. The resulting residue was dissolved with EtOAc and washed with water. The organic layer was dried over Na₂SO₄ and concentrated. The resulting residue was stirred in n-pentane, the resulting solids were filtered and thoroughly dried to afford 21 (13.0 g, 76.7% yield) as an orange solid.

tert-Butyl 3-(5-chloro-4-cyano-2-fluorophenyl)propanoate (22). To a stirred solution of 21 (2.0 g, 7.1 mmol) in EtOH (16 mL) was added 5% Pd/C (0.20 g) and the reaction mixture was stirred under a hydrogen balloon for 24 h at room temperature. The reaction mixture was filtered through Celite and washed with DCM. The organic filtrate was concentrated in vacuo to afford 22 (1.3 g, 65% yield) as a pale yellow solid.

tert-Butyl 3-(5-chloro-2-fluoro-4-(N′-hydroxycarbamimidoyl)phenyl)propanoate (23). Intermediate 23 was made from 22 using reaction conditions similar to those used for the conversion of intermediate 14 to intermediate 15. The product was stirred in hexane, filtered, and thoroughly dried to afford 23 as an off-white solid (1.0 g, 81% yield).

tert-Butyl 3-(5-chloro-4-(5-(8-chloro-6-(trifluoromethyl)imidazo[1,2-a]pyridin-2-yl)-1,2,4-oxadiazol-3-yl)-2-fluorophenyl)propanoate (24). To a stirred solution of intermediate 10 (2.5 g, 9.4 mmol) in dry DMF (20 mL) was added EDCI.HCl (1.8 g, 9.4 mmol) and HOBT (1.27 g, 9.40 mmol) at room temperature and stirred for 20 min, followed by addition of 23 (2.0 g, 6.0 mmol) with continues stirring at room temperature for 20 min. The reaction mixture was heated to 100° C. for 14 h. The reaction mixture was concentrated in vacuo. The residue was dissolved in EtOAc and washed with saturated sodium bicarbonate solution and water, dried over Na₂SO₄ and concentrated. The obtained intermediate was stirred in iPrOH, filtered, and thoroughly dried to afford 24 (2.0 g, 61% yield) as a pale yellow solid.

tert-Butyl 3-(5-chloro-2-fluoro-4-(5-(6-(trifluoromethyl)imidazo[1,2-a]pyridin-2-yl)-1,2,4-oxadiazol-3-yl)phenyl)propanoate. To a stirred solution of 24 (5.0 g, 9.2 mmol) in DCM (50 mL) was added TUFA (25 mL) at 0° C., followed by stirring at room temperature overnight. The reaction mixture was concentrated in vacuo. The residue was cooled to 0° C. and iPrOH added. The resulting solid was filtered and thoroughly dried to afford the product (2.5 g, 56%) as a light brown solid. ¹H NMR (400 MHz, DMSO-d₆) δ 12.23 (s, 1H, COOH), 9.27 (s, 1H), 9.07 (s, 1H), 8.08 (s, 1H), 7.85 (d, 1H), 7.72 (d, 1H), 2.90 (m, 2H), 2.65 (m, 2H); MS (EI) for C₁₉H₁₀Cl₂F₄N₄O₃, found 489 (MH+).

The following compounds were prepared using the same or analogous synthetic techniques in Example 2 and substituting with appropriate reagents, which were commercially available or were prepared using procedures herein or procedures known to one of ordinary skill in the art.

3-{5-Chloro-2-fluoro-4-[5-(6-iodoimidazo[1,2-a]pyridin-2-yl)-1,2,4-oxadiazol-3-yl]phenyl}propanoic acid. ¹H-NMR (400 MHz, DMSO-d₆) δ 12.35 (br s, 1H), 9.05 (s, 1H), 8.79 (s, 1H), 7.80 (d, 1H), 7.73 (d, 1H), 7.61 (m, 2H), 2.93 (t, 2H), 2.65 (t, 2H); MS (EI) for C₁₈H₁₁ClFIN₄O₃, found 513.0 (MH+).

3-{5-Chloro-4-[5-(6,8-dibromoimidazo[1,2-a]pyridin-2-yl)-1,2,4-oxadiazol-3-yl]-2-fluorophenyl}propanoic acid. ¹H-NMR (400 MHz, DMSO-d₆) δ 12.32 (br s, 1H), 9.03 (d, 1H), 8.94 (s, 1H), 8.07 (d, 1H), 7.84 (d, 1H), 7.73 (m, 2H), 2.93 (t, 2H), 2.65 (t, 2H); MS (EI) for C₁₈H₁₀Br₂ClFN₄O₃, found 544.9 (MH+).

3-{4-[5-(8-Bromo-6-methylimidazo[1,2-a]pyridin-2-yl)-1,2,4-oxadiazol-3-yl]-5-chloro-2-fluorophenyl}propanoic acid. ¹H-NMR (400 MHz, DMSO-d₆) δ 12.33 (s, 1H), 8.98 (s, 1H), 8.49 (s, 1H), 7.84 (d, 1H), 7.74 (m, 2H), 2.94 (t, 2H), 2.65 (t, 2H), 2.32 (s, 3H); MS (EI) for C₁₉H₁₃BrClFN₄O₃, found 479.0 (MH+).

3-{5-Chloro-4-[5-(8-chloro-6-methylimidazo[1,2-a]pyridin-2-yl)-1,2,4-oxadiazol-3-yl]-2-fluorophenyl}propanoic acid. ¹H-NMR (400 MHz, DMSO-d₆) δ 12.33 (s, 1H), 8.96 (s, 1H), 8.46 (s, 1H), 7.84 (d, 1H), 7.73 (d, 1H), 7.60 (s, 1H), 2.94 (t, 2H), 2.65 (t, 2H), 2.33 (s, 3H); MS (EI) for C₁₉H₁₃Cl₂FN₄O₃, found 433.0 (MH+).

3-{5-Chloro-4-[5-(6,8-difluoroimidazo[1,2-a]pyridin-2-yl)-1,2,4-oxadiazol-3-yl]-2-fluorophenyl}propanoic acid. ¹H-NMR (400 MHz, DMSO-d₆) δ 12.31 (s, 1H), 9.03 (s, 1H), 8.81 (s, 1H), 7.78 (m, 3H), 2.94 (t, 2H), 2.65 (t, 2H); MS (EI) for C₁₈H₁₀ClF₃N₄O₃, found 423.1 (MH+).

3-{-4-[5-(8-Bromo-6-cyanoimidazo[1,2-a]pyridin-2-yl)-1,2,4-oxadiazol-3-yl]-5-chloro-2-fluorophenyl}propanoic acid. ¹H-NMR (400 MHz, DMSO-d₆) δ 12.34 (br s, 1H), 9.46 (s, 1H), 9.09 (s, 1H), 8.22 (s, 1H), 7.85 (d, 1H), 7.73 (d, 1H), 2.94 (t, 2H), 2.66 (m, 2H); MS (EI) for C₁₉H₁₀BrClFN₅O₃, found 448.0 (MH+).

3-[5-Chloro-4-(5-{8-chloro-6-[(methylsulfonyl)amino]imidazo[1,2-a]pyridin-2-yl}-1,2,4-oxadiazol-3-yl)-2-fluorophenyl]propanoic acid. ¹H-NMR (400 MHz, DMSO-d₆) δ 12.35 (br s, 1H), 9.11 (s, 1H), 8.62 (s, 1H), 7.84 (d, 1H), 7.73 (d, 1H), 7.51 (s, 1H), 3.12 (s, 3H), 2.93 (t, 2H), 2.64 (t, 2H); MS (EI) for C₁₉H₁₄Cl₂FN₅O₅S, found 514.0 (MH+).

3-[5-Chloro-2-fluoro-4-(5-imidazo[1,2-a]pyridin-2-yl-1,2,4-oxadiazol-3-yl)phenyl]propanoic acid. ¹H-NMR (400 MHz, DMSO-d₆) δ 12.35 (s, 1H), 8.94 (s, 1H), 8.67 (d, 1H), 7.82 (d, 1H), 7.73 (t, 2H), 7.45 (m, 1H), 7.10 (t, 1H), 2.94 (t, 2H), 2.65 (t, 2H); MS (EI) for C₁₈H₁₂ClFN₄O₃, found 387 (MH+).

3-{5-Chloro-4-[5-(8-chloro-6-nitroimidazo[1,2-a]pyridin-2-yl)-1,2,4-oxadiazol-3-yl]-2-fluorophenyl}propanoic acid. ¹H-NMR (400 MHz, DMSO-d₆) δ 12.33 (br s, 1H), 9.98 (dd, 1H), 9.19 (s, 1H), 8.41-8.40 (dd, 1H), 7.87-7.84 (d, 1H), 7.75-7.73 (d, 1H), 2.96-2.92 (t, 2H), 2.67-2.63 (t, 2H); MS (EI) for C₁₈H₁₀Cl₂FN₅O₅, found 466 (MH+).

3-{5-Chloro-4-[5-(6,8-dichloro-7-methylimidazo[1,2-a]pyridin-2-yl)-1,2,4-oxadiazol-3-yl]-2-fluorophenyl}propanoic acid. ¹H-NMR (400 MHz, DMSO-d₆) δ 12.15 (br s, 1H), 8.96 (s, 1H), 8.87 (s, 1H), 7.82 (d, 1H), 7.71 (d, 1H), 2.93 (t, 2H), 2.64 (t, 2H), 2.52 (s, 3H); MS (EI) for C₁₉H₁₂Cl₃FN₄O₃, found 469.0 (MH+).

3-(5-Chloro-4-{5-[6-chloro-7-(methyloxy)imidazo[1,2-a]pyridin-2-yl]-1,2,4-oxadiazol-3-yl}-2-fluorophenyl)propanoic acid. ¹H-NMR (400 MHz, DMSO-d₆) δ 12.15 (br s, 1H), 8.93 (s, 1H), 8.67 (s, 1H), 7.81 (d, 1H), 7.71 (d, 1H), 7.31 (s, 1H), 3.98 (s, 3H), 2.93 (t, 2H), 2.64 (t, 2H); MS (EI) for C₁₉H₁₃Cl₂FN₄O₄, found 451.0 (MH+).

3-{5-Chloro-4-[5-(8-chloroimidazo[1,2-a]pyridin-2-yl)-1,2,4-oxadiazol-3-yl]-2-fluorophenyl}propanoic acid. ¹H-NMR (400 MHz, DMSO-d₆) δ 12.35 (br s, 1H), 9.06 (s, 1H), 8.66 (d, 1H), 7.82 (d, 1H), 7.72 (d, 1H), 7.66 (d, 1H), 7.08 (dd, 1H), 2.93 (t, 2H), 2.64 (t, 2H); MS (EI) for C₁₈H₁₁Cl₂FN₄O₃, found 421.0 (MH+).

3-{5-Chloro-4-[5-(8-chloro-6-cyanoimidazo[1,2-a]pyridin-2-yl)-1,2,4-oxadiazol-3-yl]-2-fluorophenyl}propanoic acid. ¹H-NMR (400 MHz, DMSO-d₆) δ 12.18 (br s, 1H), 9.43 (s, 1H), 9.08 (s, 1H), 8.11 (d, 1H), 7.84 (d, 1H), 7.72 (m, 2H), 2.93 (t, 2H), 2.65 (t, 2H); MS (EI) for C₁₉H₁₀Cl₂FN₅O₃, found 446.0 (MH+).

3-{4-[5-(6-Bromo-8-chloroimidazo[1,2-a]pyridin-2-yl)-1,2,4-oxadiazol-3-yl]-5-chloro-2-fluorophenyl}propanoic acid. ¹H-NMR (400 MHz, DMSO-d₆) δ 12.32(br s, 1H), 9.00 (d, 1H), 8.93 (s, 1H), 7.95 (d, 1H), 7.83 (d, 1H), 7.72 (d, 1H), 2.93 (t, 2H), 2.65 (t, 2H); MS (EI) for C₁₈H₁₀BrCl₂FN₄O₃, found 501.0 (MH+).

3-(5-Chloro-4-{5-[8-chloro-6-(methyloxy)imidazo[1,2-a]pyridin-2-yl]-1,2,4-oxadiazol-3-yl}-2-fluorophenyl)propanoic acid. ¹H-NMR (400 MHz, DMSO-d₆) δ 12.33(br s, 1H), 8.89 (s, 1H), 8.38 (d, 1H), 7.82 (d, 1H), 7.73 (d, 1H), 7.58 (d, 1H), 3.84 (s, 3H), 2.93 (t, 2H), 2.65 (t, 2H); MS (D) for C₁₉H₁₃Cl₂FN₄O₄, found 451.0 (MH+).

3-{5-Chloro-4-[5-(6,8-dibromo-5-methylimidazo[1,2-a]pyridin-2-yl)-1,2,4-oxadiazol-3-yl]-2-fluorophenyl}propanoic acid. ¹H-NMR (400 MHz, DMSO-d₆) δ 12.32(br s, 1H), 9.12 (s, 1H), 8.09 (s, 1H), 7.86 (d, 1H), 7.73 (d, 1H), 2.93 (t, 2H), 2.81 (s, 3H), 2.65 (t, 2H); MS (D) for C₁₉H₁₂Br₂ClFN₄O₃, found 558.9 (MH+).

3-[5-Chloro-4-(5-{8-chloro-6-[(4-methylphenyl)oxy]imidazo[1,2-a]pyridin-2-yl}-1,2,4-oxadiazol-3-yl)-2-fluorophenyl]propanoic acid. ¹H-NMR (400 MHz, DMSO-d₆) δ 12.35 (br s, 1H), 8.92 (s, 1H), 8.41 (d, 1H), 7.82 (d, 1H), 7.72 (d, 1H), 7.69 (d, 1H), 7.26 (d, 2H), 7.10 (d, 2H), 2.93 (t, 2H), 2.64 (t, 2H), 2.23 (s, 3H); MS (D) for C₂₅H₁₇Cl₂FN₄O₄, found 527.1 (MH+).

3-(4-{5-[8-Chloro-6-(trifluoromethyl)imidazo[1,2-a]pyridin-2-yl]-1,2,4-oxadiazol-3-yl}-2,5-difluorophenyl)propanoic acid. MS (D) for C₁₉H₁₀ClF₅N₄O₃, found 473 (MH+).

3-(4-{5-[8-Bromo-6-(trifluoromethyl)imidazo[1,2-a]pyridin-2-yl]-1,2,4-oxadiazol-3-yl}-5-chloro-2-fluorophenyl)propanoic acid. MS (D) for C₁₉H₁₀BrClF₄N₄O₃, found 534 (MH+).

3-(4-{5-[8-Bromo-6-(trifluoromethyl)imidazo[1,2-a]pyridin-2-yl]-1,2,4-oxadiazol-3-yl}-2,5-dichlorophenyl)propanoic acid. MS (D) for C₁₉H₁₀BrCl₂F₃N₄O₃, found 549 (MH+).

3-(2-Chloro-4-{5-[8-chloro-6-(trifluoromethyl)imidazo[1,2-a]pyridin-2-yl]-1,2,4-oxadiazol-3-yl}-5-methylphenyl)propanoic acid. MS (D) for C₂₀H₁₃Cl₂F₃N₄O₃, found 485 (MH+).

### Example 3

### 2-(4-(5-(8-Chloro-6-(trifluoromethyl)-imidazo[1,2-a]pyridin-2-yl)-1,2,4-oxadiazol-3-yl)-3-methylphenyl)cyclopropanecarboxylic acid

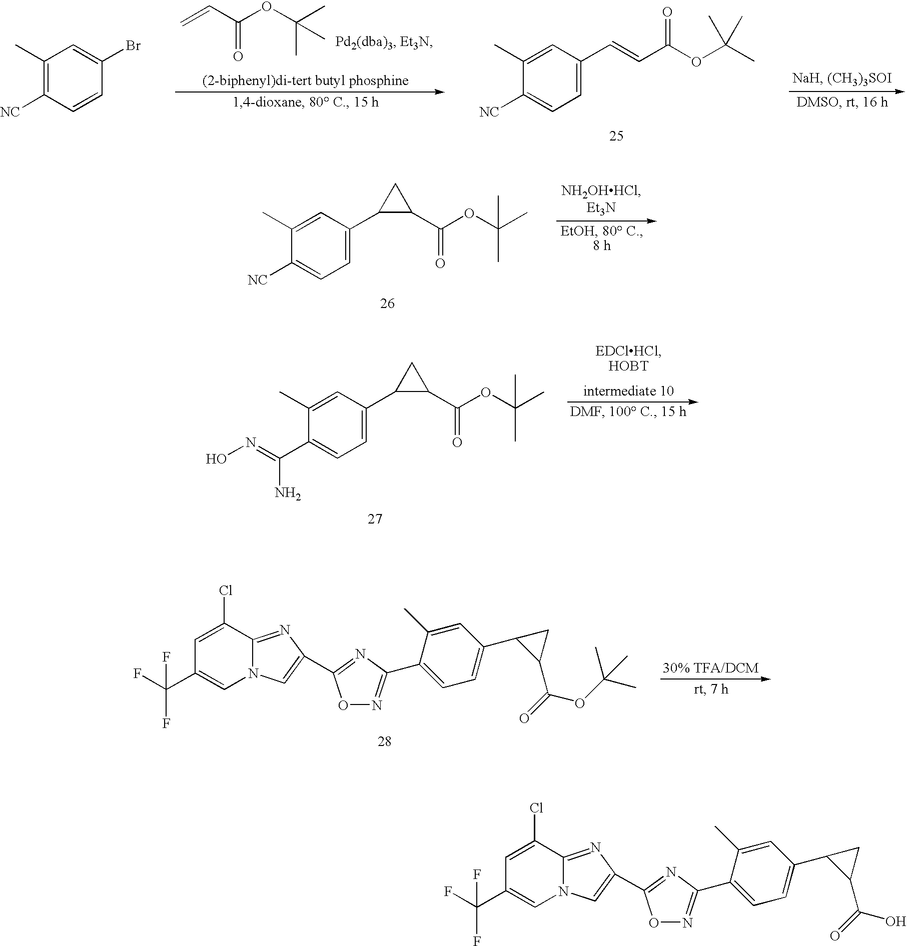

tert-Butyl 3-(3-methyl-4-cyanophenyl)acrylate (25). Intermediate 25 was made from 4-bromo-2-methylbenzonitrile using conditions similar to those used in the conversion of intermediate 11 to intermediate 13. The crude intermediate was stirred in pentane, filtered, and thoroughly dried to afford 25 as an off-white solid (15 g, 78%).

tert-Butyl 2-(4-cyano-3-methylphenyl)cyclopropanecarboxylate (26). To a stirred solution of 60% NaH (2.4 g, 0.062 moles) in dry DMSO (160 mL) was added trimethylsulfoxonium iodide (13.64 g, 0.06200 moles) slowly over 45 min. After addition was complete, the reaction mixture was stirred at room temperature for 1 h and then olefinic ester 25 was added slowly over 45 min. The reaction mixture was stirred 16 h at room temperature and diluted with ice cold water (200 mL). The product was extracted into ether (4×150 mL) and the ether layers were combined and washed with water and saturated NaCl solution. The organic layers were combined, dried over Na₂SO₄, and concentrated in vacuo. The crude compound was purified by column chromatography (1% EtOAc/hexane) to afford 26 (3.4 g, 21%) as an oily liquid. ¹H NMR (400 MHz, CDCl₃) δ 7.5 (d, 1H), 7.1 (s, 1H), 6.9 (d, 1H), 2.5 (s, 3H), 2.4 (m, 1H), 1.82 (m, 1H), 1.6 (m, 1H), 1.22 (m, 1H).

tert-Butyl 2-(4-amidino-3-methylphenyl)cyclopropanecarboxylate (27). Intermediate 27 was made from 26 using conditions similar to those used in the conversion of intermediate 14 to intermediate 15 to afford hydroxyimidate 27 as a semi solid (3.1 g, 83%).

tert-Butyl 3-(3-methyl-4-(5-(8-chloro-6-(trifluoromethyl)-imidazo[1,2-a]pyridin-2-yl)-1,2,4-oxadiazol-3-yl)phenyl)propanoate (28). Intermediate 28 was made from 27 using conditions similar to those used in the conversion of intermediate 15 to intermediate 16. The product was purified by column chromatography (15% EtOAc/hexane) to afford 28 as a white solid (1.8 g, 32%).

2-(4-(5-(8-Chloro-6-(trifluoromethyl)-imidazo[1,2-a]pyridin-2-yl)-1,2,4-oxadiazol-3-yl)-3-methylphenyl)cyclopropanecarboxylic acid. The product was made from 28 using conditions similar to those used to prepare the final product in Example 1, affording a white solid (0.3 g, 70% yield). ¹H NMR (400 MHz, DMSO-d₆) δ 12.4 (br s, —COOH), 9.4 (s, 1H), 9.0 (s, 1H), 8.1 (s, 1H), 7.9 (d, 1H), 7.3 (s, 1H), 7.2 (d, 1H), 2.6 (s, 3H), 2.4 (m, 1H), 1.9 (m, 1H), 1.5 (m, 2H); MS (EI) for C₂₁H₁₄ClF₃N₄O₃, found 463 (MH+).

The following compounds were prepared using the same or analogous synthetic techniques in Example 3 and substituting with appropriate reagents, which were commercially available or were prepared using procedures herein or procedures known to one of ordinary skill in the art.

2-(5-Chloro-4-{5-[8-chloro-6-(trifluoromethyl)imidazo[1,2-a]pyridin-2-yl]-1,2,4-oxadiazol-3-yl}-2-fluorophenyl)cyclopropanecarboxylic acid. ¹H-NMR (400 MHz, DMSO-d₆) δ 12.55 (s, 1H), 9.35 (s, 1H), 9.07 (s, 1H), 8.09 (d, 1H), 7.85 (d, 1H), 7.55 (d, 1H), 2.55 (m, 1H), 2.07 (m, 1H), 1.65 (m, 1H), 1.50 (m, 1H); MS (EI) for C₂₀H₁₀C₁₂F₄N₄O₃, found 499 (MH−).

2-(4-{5-[8-Chloro-6-(trifluoromethyl)imidazo[1,2-a]pyridin-2-yl]-1,2,4-oxadiazol-3-yl}-2,5-difluorophenyl)cyclopropanecarboxylic acid. MS (EI) for C₂₀H₁₀ClF₅N₄O₃, found 485 (MH+).

2-(4-{5-[8-Bromo-6-(trifluoromethyl)imidazo[1,2-a]pyridin-2-yl]-1,2,4-oxadiazol-3-yl}-5-chloro-2-fluorophenyl)cyclopropanecarboxylic acid. MS (EI) for C₂₀H₁₀BrClF₄N₄O₃, found 546.8 (MH+).

2-(4-{5-[8-Chloro-6-(trifluoromethyl)imidazo[1,2-a]pyridin-2-yl]-1,2,4-oxadiazol-3-yl}-3-fluorophenyl)cyclopropanecarboxylic acid. MS (EI) for C₂₀H₁₁ClF₄N₄O₃, found 467 (MH+).

2-(3-Chloro-4-{5-[8-chloro-6-(trifluoromethyl)imidazo[1,2-a]pyridin-2-yl]-1,2,4-oxadiazol-3-yl}phenyl)cyclopropanecarboxylic acid. MS (EI) for C₂₀H₁₁Cl₂F₃N₄O₃, found 483 (MH+).

2-[4-{5-[8-Chloro-6-(trifluoromethyl)imidazo[1,2-a]pyridin-2-yl]-1,2,4-oxadiazol-3-yl}-3-(trifluoromethyl)phenyl]cyclopropanecarboxylic acid. ¹H-NMR (400 MHz, DMSO-d₆) δ 12.42 (br s, 1H), 9.25 (s, 1H), 9.03 (s, 1H), 8.04 (s, 1H), 7.90 (d, 2H), 7.63 (d, 1H), 2.02 (m, 1H), 1.57 (m, 1H), 0.82 (m, 1H); MS (EI) for C₂₁H₁₁ClF₆N₄O₃, found 517 (MH+).

2-(3-Chloro-4-{5-[8-chloro-6-(trifluoromethyl)imidazo[1,2-a]pyridin-2-yl]-1,2,4-oxadiazol-3-yl}phenyl)cyclopropanecarboxamide (prepared from the acid, which is itself prepared using procedures in Example 3, by treating with NH₃ using conditions known to one of ordinary skill in the art). MS (EI) for C₂₀H₁₂Cl₂F₃N₅O₂, found 482 (MH+).

[2-(3-Chloro-4-{5-[8-chloro-6-(trifluoromethyl)imidazo[1,2-a]pyridin-2-yl]-1,2,4-oxadiazol-3-yl}phenyl)cyclopropyl]methanol (prepared by reducing the acid, which is itself prepared using the procedures described in Example 3). ¹H-NMR (400 MHz, DMSO-d₆) 9.33 (s, 1H), 9.05 (s, 1H), 8.06 (s, 1H), 7.92 (d, 1H), 7.43 (s, 1H), 7.26 (d, 1H), 4.69 (t, 1H), 3.52 (m, 1H), 3.36 (m, 1H), 1.95 (m, 1H), 1.42 (m, 1H), 1.02 (m, 2H).

2-(2-Chloro-4-{5-[8-chloro-6-(trifluoromethyl)imidazo[1,2-a]pyridin-2-yl]-1,2,4-oxadiazol-3-yl}phenyl)cyclopropanecarboxylic acid. ¹H-NMR (400 MHz, DMSO-d₆) δ 12.48 (br, s, 1H), 9.31 (s, 1H), 9.06 (s, 1H), 8.11 (s, 1H), 8.07 (s, 1H), 8.00 (d, 1H), 7.40 (s, 1H), 2.66 (m, 1H), 1.89 (m, 1H), 1.52 (m, 2H); MS (EI) for C₂₀H₁₁Cl₂F₃N₄O₃, found 483 (MH+).

2-(4-{5-[8-Chloro-6-(trifluoromethyl)imidazo[1,2-a]pyridin-2-yl]-1,2,4-oxadiazol-3-yl}-2-fluoro-5-methylphenyl)cyclopropanecarboxylic acid. MS (EI) for C₂₁H₁₃ClF₄N₄O₃, found 481 (MH+).

2-(4-{5-[8-Chloro-6-(trifluoromethyl)imidazo[1,2-a]pyridin-2-yl]-1,2,4-oxadiazol-3-yl}phenyl)cyclopropanecarboxylic acid. MS (EI) for C₂₀H₁₂ClF₃N₄O₃, found 449 (MH+).

2-(4-{5-[8-Chloro-6-(trifluoromethyl)imidazo[1,2-a]pyridin-2-yl]-1,2,4-oxadiazol-3-yl}-2-fluorophenyl)cyclopropanecarboxylic acid. MS (EI) for C₂₀H₁₁ClF₄N₄O₃, found 467 (MH+).

2-(4-{5-[8-Chloro-6-(trifluoromethyl)imidazo[1,2-a]pyridin-2-yl]-1,2,4-oxadiazol-3-yl}-2-methylphenyl)cyclopropanecarboxylic acid. MS (EI) for C₂₁H₁₄ClF₃N₄O₃, found 463 (MH+).

2-(2,5-Dichloro-4-{5-[8-chloro-6-(trifluoromethyl)imidazo[1,2-a]pyridin-2-yl]-1,2,4-oxadiazol-3-yl}phenyl)cyclopropanecarboxylic acid. MS (EI) for C₂₀H₁₀Cl₃F₃N₄O₃, found 517 (MH+).

2-(4-{5-[8-Bromo-6-(trifluoromethyl)imidazo[1,2-a]pyridin-2-yl]-1,2,4-oxadiazol-3-yl}-2,5-dichlorophenyl)cyclopropanecarboxylic acid. MS (EI) for C₂₀H₁₀BrCl₂F₃N₄O₃, found 563 (MH+).

### Example 4

### 3-Chloro-4-(5-(8-chloro-6-(trifluoromethyl)-imidazo[1,2-a]pyridin-2-yl)-1,2,4-oxadiazol-3-yl)benzenaminemethanesulfonate

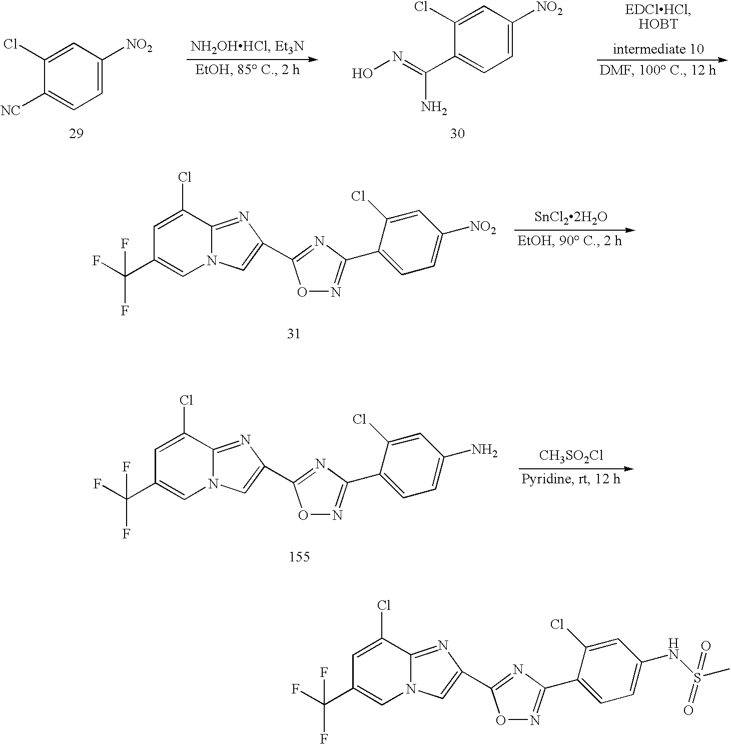

2-Chloro-N′-hydroxy-4-nitrobenzamidine (30). To a stirred solution of 2-chloro-4-nitrobenzonitrile 29 (5.0 g, 0.027 moles) in EtOH (30 mL) was added hydroxylamine hydrochloride (9.5 g, 0.14 moles) followed by triethylamine (13.8 g, 0.136 mol) at room temperature. After stirring at 85° C. for 2 h, the reaction mixture was concentrated in vacuo and water added to the residue. The product was extracted into EtOAc. The organic layers were combined, washed with water (2×), combined again, dried over Na₂SO₄, and concentrated. The crude product was purified by column chromatography (30% EtOAc/hexane), affording hydroxyimidate 30 (4.0 g, 68% yield) as a yellow solid.

8-Chloro-2-(3-(2-chloro-4-nitrophenyl)-1,2,4-oxadiazol-5-yl)-6-(trifluoromethyl)-imidazo[1,2-a]pyridine (31). To a stirred solution of intermediate 10 (7.34 g, 0.0277 mol) in DMF (20 mL) was added EDCI.HCl (5.38 g, 0.0281 mol) and HOBT (3.76 g, 0.0279 mol). The mixture was stirred at room temperature for 30 min, followed by addition of hydroxyimidate 30 (4.0 g, 0.019 mol). The reaction mixture was stirred at room temperature for 4 h, followed by heating to 100° C. for 12 h. The reaction mixture was concentrated in vacuo and the residue was purified by column chromatography (30% EtOAc/hexane) to afford 31 (4.05 g, 48.0% yield) as an off-white solid.

3-Chloro-4-(5-(8-chloro-6-(trifluoromethyl)-imidazo[1,2-a]pyridin-2-yl)-1,2,4-oxadiazol-3-yl)benzenamine. To a stirred solution of intermediate 31 (4.05 g, 0.00911 moles) in EtOH (25 mL) was added SnCl₂.2H₂O (10.28 g, 0.04556 moles) at room temperature. The resulting reaction mixture was heated to 90° C. for 2 h, and then concentrated in vacuo. The residue was cooled to 0° C. and the pH adjusted to 10-12 with 1 M NaOH and the product was extracted into EtOAc. The organic layers were combined, washed with water and saturated NaCl solution. The organic layers were combined again, dried over Na₂SO₄, and concentrated in vacuo. The crude reaction mixture was suspended in a small amount of iPrOH and stirred for 15 min. The resulting solid was filtered and thoroughly dried to afford the product 155 (3.6 g, 95% yield) as a yellow solid. ¹H-NMR (400 MHz, DMSO-d₆) δ 9.35 (s, 1H), 9.00 (s, 1H), 8.05 (s, 1H), 7.85 (d, 1H), 6.80 (3, 1H), 6.65 (d, 1H), 6.10 (br s, 2H).

3-Chloro-4-(5-(8-chloro-6-(trifluoromethyl)-imidazo[1,2-a]pyridin-2-yl)-1,2,4-oxadiazol-3-yl)benzenaminemethanesulfonate. To a stirred solution of the amine from the previous step (2.9 g, 0.0070 mol) in pyridine (20 mL) was added methanesulfonyl chloride (1.2 g, 0.011 moles) dropwise at 0° C., followed by stirring at room temperature for 2 h. After completion, the reaction mixture was poured into ice cold water to which saturated sodium bicarbonate solution was added. The product was extracted into EtOAc. The organic layers were combined, washed with 1 N HCl, water, and a saturated NaCl solution. The organic layers were collected, dried over Na₂SO₄, and concentrated in vacuo. The crude reaction mixture was suspended in a small amount of iPrOH and stirred for 15 min. The resulting solid was filtered and thoroughly dried to afford the product (2.5 g, 73%) as a pale yellow solid. ¹H NMR (400 MHz, DMSO-d₆) δ 10.5 (s, —NH), 9.3 (s, 1H), 9.0 (s, 1H), 8.02 (m, 2H), 7.42 (s, 1H), 7.40 (d, 1H), 3.1 (s, 3H); MS (EI) for C₁₇H₁₀Cl₂F₃N₅O₃S, found 492 (MH+).

The following compounds were prepared using the same or analogous synthetic techniques in Example 4 and substituting with appropriate reagents, which were commercially available or were prepared using procedures herein or procedures known to one of ordinary skill in the art.

N-{3-Chloro-4-[5-(8-chloro-6-cyanoimidazo[1,2-a]pyridin-2-yl)-1,2,4-oxadiazol-3-yl]phenyl}methanesulfonamide. ¹H-NMR (400 MHz, DMSO-d₆) δ 10.52 (br s, 1H), 9.43 (s, 1H), 9.06 (s, 1H), 8.11 (s, 1H), 8.06-8.04 (d, 1H), 7.46 (s, 1H), 7.39-7.37 (dd, 1H), 3.19 (s, 3H); MS (EI) for C₁₇H₁₀Cl₂N₆O₃S, found 447 (MH+).

N-{4-[5-(8-Bromo-6-cyanoimidazo[1,2-a]pyridin-2-yl)-1,2,4-oxadiazol-3-yl]-3-chlorophenyl}methanesulfonamide. ¹H-NMR (400 MHz, DMSO-d₆) δ 10.51 (br s, 1H), 9.46 (s, 1H), 9.07 (s, 1H), 8.22 (s, 1H), 8.07-8.04 (d, 1H), 7.46 (s, 1H), 7.39-7.37 (dd, 1H), 3.19 (s, 3H); MS (EI) for C₁₇H₁₀BrClN₆O₃S, found 493 (MH+).

N-{3-Chloro-4-[5-(8-chloro-6-methylimidazo[1,2-a]pyridin-2-yl)-1,2,4-oxadiazol-3-yl]phenyl}methanesulfonamide. ¹H-NMR (400 MHz, DMSO-d₆) δ 10.50 (br s, 1H), 8.93 (s, 1H), 8.45 (s, 1H), 8.03 (d, 1H), 7.58 (d, 1H), 7.45 (d, 2H), 7.38 (dd, 1H), 3.18 (s, 3H), 2.32 (s, 3H); MS (EI) for C₁₇H₁₃Cl₂N₅O₃S, found 438.0 (MH+).

N-{4-[5-(8-Bromo-6-methylimidazo[1,2-a]pyridin-2-yl)-1,2,4-oxadiazol-3-yl]-3-chlorophenyl}methanesulfonamide. ¹H-NMR (400 MHz, DMSO-d₆) δ 10.50 (br s, 1H), 8.95 (s, 1H), 8.48 (m, 1H), 8.03 (d, 1H), 7.79 (s, 1H), 7.45 (d, 2H), 7.36 (dd, 1H), 3.18 (s, 3H), 2.32 (s, 3H); MS (EI) for C₁₇H₁₃BrClN₅O₃S, found 484.0 (MH+).

N-(2-Chloro-4-{5-[8-chloro-6-(trifluoromethyl)imidazo[1,2-a]pyridin-2-yl]-1,2,4-oxadiazol-3-yl}-6-fluorophenyl)methanesulfonamide. MS (EI) for C₁₇H₉Cl₂F₄N₅O₃S, found 510 (MH+).

N-(5-Chloro-4-{5-[8-chloro-6-(trifluoromethyl)imidazo[1,2-a]pyridin-2-yl]-1,2,4-oxadiazol-3-yl}-2-fluorophenyl)methanesulfonamide. ¹H-NMR (400 MHz, DMSO-d₆) δ 10.36 (br, s, 1H), 9.34 (s, 1H), 9.06 (s, 1H), 8.07 (s, 1H), 8.00-7.97 (d, 1H), 7.74-7.73 (d, 1H), 3.26 (s, 3H); MS (EI) for C₁₇H₉Cl₂F₄N₅O₃S, found 510 (MH+).

N-(4-{5-[8-Chloro-6-(trifluoromethyl)imidazo[1,2-a]pyridin-2-yl]-1,2,4-oxadiazol-3-yl}-3-fluorophenyl)methanesulfonamide. MS (EI) for C₁₇H₁₀ClF₄N₅O₃S, found 476 (MH+).

N-(3-Chloro-4-{5-[8-chloro-6-(trifluoromethyl)imidazo[1,2-a]pyridin-2-yl]-1,2,4-oxadiazol-3-yl}phenyl)glycine. MS (EI) for C₁₈H₁₀Cl₂F₃N₅O₃, found 472 (MH+).

N-(3-Chloro-4-{5-[8-chloro-6-(trifluoromethyl)imidazo[1,2-a]pyridin-2-yl]-1,2,4-oxadiazol-3-yl}phenyl)-beta-alanine MS (EI) for C₁₉H₁₂Cl₂F₃N₅ O₃, found 486 (MH+).

N-(4-{5-[8-Chloro-6-(trifluoromethyl)imidazo[1,2-a]pyridin-2-yl]-1,2,4-oxadiazol-3-yl}-3-methylphenyl)methanesulfonamide. MS (EI) for C₁₈H₁₃ClF₃N₅O₃S, found 472 (MH+).

N-(4-{5-[8-Chloro-6-(trifluoromethyl)imidazo[1,2-a]pyridin-2-yl]-1,2,4-oxadiazol-3-yl}-2-fluorophenyl)methanesulfonamide. MS (EI) for C₁₇H₁₀ClF₄N₅O₃S, found 476 (MH+).

N-(3-Chloro-4-{5-[8-chloro-6-(trifluoromethyl)imidazo[1,2-a]pyridin-2-yl]-1,2,4-oxadiazol-3-yl}phenyl)-2-(diethylamino)ethanesulfonamide. MS (EI) for C₂₂H₂₁Cl₂F₃N₆ O₃S found 577 (MH+).

N-(4-{5-[8-Chloro-6-(trifluoromethyl)imidazo[1,2-a]pyridin-2-yl]-1,2,4-oxadiazol-3-yl}-2-fluoro-5-methylphenyl)methanesulfonamide. MS (EI) for C₁₈H₁₂ClF₄N₅ O₃S, found 490 (MH+).

N-(3-Chloro-4-{5-[8-chloro-6-(trifluoromethyl)imidazo[1,2-a]pyridin-2-yl]-1,2,4-oxadiazol-3-yl}phenyl)ethenesulfonamide. MS (EI) for C₁₈H₁₀Cl₂F₃N₅O₃S, found 504 (MH+).

N-(4-{5-[8-Chloro-6-(trifluoromethyl)imidazo[1,2-a]pyridin-2-yl]-1,2,4-oxadiazol-3-yl}-2,6-difluorophenyl)methanesulfonamide. MS (EI) for C₁₇H₉ClF₅N₅O₃S, found 494 (MH+).

N-(4-{5-[8-Chloro-6-(trifluoromethyl)imidazo[1,2-a]pyridin-2-yl]-1,2,4-oxadiazol-3-yl}phenyl)methanesulfonamide. MS (EI) for C₁₇H₁₁ClF₃N₅O₃S, found 458 (MH+).

N-(4-{5-[8-Chloro-6-(trifluoromethyl)imidazo[1,2-a]pyridin-2-yl]-1,2,4-oxadiazol-3-yl}-2-methylphenyl)methanesulfonamide. MS (EI) for C₁₈H₁₃ClF₃N₅O₃S, found 472 (MH+).

N-(2-Chloro-4-{5-[8-chloro-6-(trifluoromethyl)imidazo[1,2-a]pyridin-2-yl]-1,2,4-oxadiazol-3-yl}phenyl)methanesulfonamide. MS (EI) for C₁₇H₁₀Cl₂F₃N₅O₃S, found 492 (MH+).

2-[(3-Chloro-4-{5-[8-chloro-6-(trifluoromethyl)imidazo[1,2-a]pyridin-2-yl]-1,2,4-oxadiazol-3-yl}phenyl)amino]ethanol. MS (EI) for C₁₈H₁₂Cl₂F₃N₅O₂, found 458 (MH+).

2-[(5-Chloro-4-{5-[8-chloro-6-(trifluoromethyl)imidazo[1,2-a]pyridin-2-yl]-1,2,4-oxadiazol-3-yl}-2-fluorophenyl)amino]ethanol. MS (EI) for C₁₈H₁₁Cl₂F₄N₅O₂, found 476.0 (MH+).

3-[(2,5-Dichloro-4-{5-[8-chloro-6-(trifluoromethyl)imidazo[1,2-a]pyridin-2-yl]-1,2,4-oxadiazol-3-yl}phenyl)amino]propane-1,2-diol. MS (EI) for C₁₉H₁₃Cl₃F₃N₅O₃, found 522.0 (MH+).

2-[(2,5-Dichloro-4-{5-[8-chloro-6-(trifluoromethyl)imidazo[1,2-a]pyridin-2-yl]-1,2,4-oxadiazol-3-yl}phenyl)amino]ethanol. MS (EI) for C₁₈H₁₁Cl₃F₃N₅O₂, found 492 (MH+).

1-[(2,5-Dichloro-4-{5-[8-chloro-6-(trifluoromethyl)imidazo[1,2-a]pyridin-2-yl]-1,2,4-oxadiazol-3-yl}phenyl)amino]propan-2-ol. MS (EI) for C₁₉H₁₃Cl₃F₃N₅O₂, found 506 (MH+).

2,5-dichloro-4-{5-[8-chloro-6-(trifluoromethyl)imidazo[1,2-a]pyridin-2-yl]-1,2,4-oxadiazol-3-yl}aniline. MS (EI) for C₁₆H₇C₁₃F₃N₅O, found 447.8 (MH+).

### Example 5

### 1-(4-(5-(8-Bromo-6-(trifluoromethyl)imidazo[1,2-a]pyridin-2-yl)-1,2,4-oxadiazol-3-yl)-3-chlorobenzyl)azetidine-3-carboxylic acid

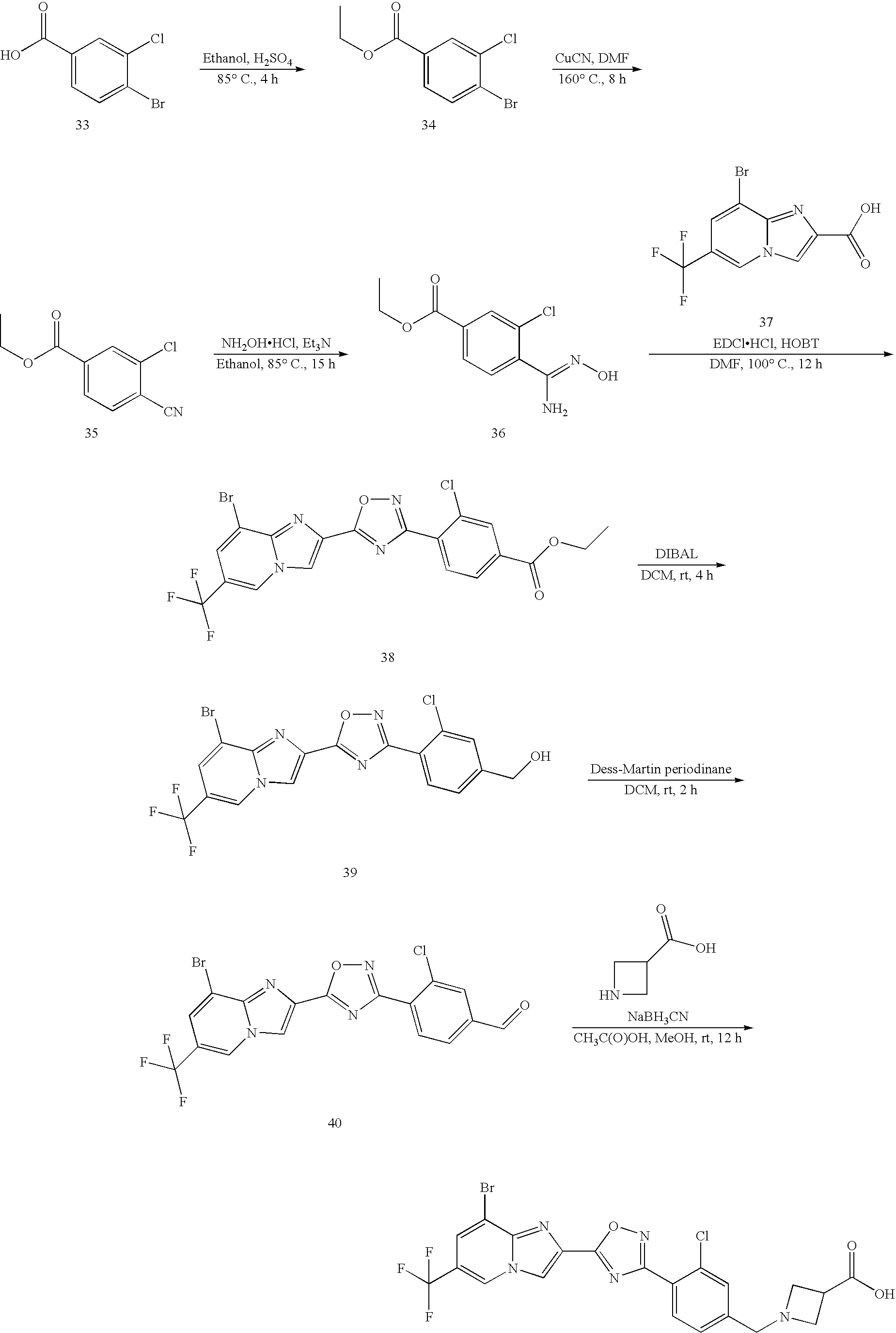

Ethyl 3-chloro-4-bromo benzoate (34). To a stirred solution of intermediate 33 (10 g, 43 mmol) in EtOH (50 mL) was added concentrated H₂SO₄ (20 mL) at 0° C. After the addition was complete, the reaction was stirred at 85° C. for 4 h. After completion, the reaction mixture was concentrated in vacuo and the residue was dissolved in DCM. The organic layer was washed with water, aqueous sodium bicarbonate solution, and saturated NaCl. The organic layers were combined, dried over Na₂SO₄, and concentrated in vacuo to afford the title intermediate 34 as an off-white solid (10 g, yield 88%).

Ethyl 3-chloro-4-cyanobenzoate (35). To a stirred solution of intermediate 34 (10 g, 38 mmol) in DMF (80 mL) was added cuprous cyanide (6.75 g, 75.4 mmol) and the mixture heated to 160° C. for 8 h. After completion, the reaction mixture was diluted with water and filtered. Ethyl acetate was added to the filtrate and the product was extracted into the organic layer. The combined organic layers were washed with saturated NaCl solution. The organic layer was dried over Na₂SO₄ and concentrated to afford the title intermediate 35 as a light yellow solid (6.0 g, yield 75%).

Ethyl 3-chloro-4-(N′-hydroxycarbamimidoyl)benzoate (36). To a stirred solution of hydroxylamine hydrochloride (7.72 g, 112 mmol) in EtOH (60 mL) was added triethylamine (14.14 g, 139.7 mmol). After stirring for 30 min, intermediate 35 (6.0 g, 29 mmol) was added and the reaction mixture was stirred at 85° C. for 15 h. After completion, solvent was removed in vacuo and the residue was dissolved in EtOAc. The organic layer was washed with water, dried over Na₂SO₄, and concentrated to afford the title intermediate 36 as a light yellow solid (5.0 g, yield 72%).

Ethyl 4-(5-(8-bromo-6-(trifluoromethyl)imidazo[1,2-a]pyridin-2-yl)-1,2,4-oxadiazol-3-yl)-3-chlorobenzoate (38). Intermediate 37 was synthesized from 2-amino-3-bromo-5-trifluoromethyl pyridine in a manner analogous to Intermediate 10. To a stirred solution of intermediate 37 (3.5 g, 11 mmol) in DMF (20 mL) was added EDCI.HCl (2.2 g, 12 mmol) and HOBT (1.5 g, 11 mmol). After stirring for 15 min, intermediate 36 (2.5 g, 10 mmol) was added and the reaction mixture was stirred at 100° C. for 12 h. After completion, the reaction mixture was concentrated and the residue was dissolved in EtOAc. The organic layer was washed with saturated sodium bicarbonate solution, water, dried over Na₂SO₄ and concentrated. The resulting solid was washed with iPrOH to afford the title intermediate 38 as white solid (2.0 g, yield 37%).

(4-(5-(8-Bromo-6-(trifluoromethyl)imidazo[1,2-a]pyridin-2-yl)-1,2,4-oxadiazol-3-yl)-3-chlorophenyl)methanol (39). To a stirred solution of intermediate 38 (1 g, 2 mmol) in DCM (20 mL) was added DIBAL (1.1 g, 7.7 mmol) dropwise at 0° C. The reaction mixture was stirred at 0° C. for 1 h and then at room temperature for 3 h. After completion, the reaction mixture was quenched with saturated ammonium chloride solution at 0° C. The product was extracted into EtOAc. The combined organic layers were washed with water, saturated NaCl, dried over Na₂SO₄, and concentrated to afford the title intermediate 39 as a white solid (700 mg, yield 76%).

4-(5-(8-Bromo-6-(trifluoromethyl)imidazo[1,2-a]pyridin-2-yl)-1,2,4-oxadiazol-3-yl)-3-chlorobenzaldehyde (40). To a stirred solution of intermediate 39 (700 mg, 1.47 mmol) in DCM (15 mL) was added Dess-Martin periodinane reagent (1.25 g, 2.94 mmol) and the resulting mixture stirred at room temperature for 2 h. After completion, the reaction mixture was diluted with DCM and washed with sodium thiosulphate, saturated sodium bicarbonate, and saturated NaCl solution. The organic layers were collected, dried over Na₂SO₄, and concentrated in vacuo to afford the title intermediate 40 as a light yellow solid (500 mg, yield 72%).

1-(4-(5-(8-Bromo-6-(trifluoromethyl)imidazo[1,2-a]pyridin-2-yl)-1,2,4-oxadiazol-3-yl)-3-chlorobenzyl)azetidine-3-carboxylic acid. To a stirred solution of intermediate 40 (0.2 g, 0.4 mmol) in MeOH (8 mL) was added azetidine-3-carboxylic acid (43 mg, 0.42 mmol) and acetic acid (0.2 mL). After stirring for 30 min, a solution of sodium cyanoborohydride (13 mg, 0.20 mmol) in MeOH (3 mL) was added. The reaction mixture was stirred at room temperature for 12 h. The suspended solid was collected by filtration, washed with MeOH, and dried completely to afford the title compound as a white solid (100 mg, yield 43%). ¹H NMR (400 MHz, DMSO-d₆) δ 9.35 (s, 1H), 9.11 (s, 1H), 8.20 (s, 1H), 8.00 (d, 1H), 7.60 (s, 1H); 7.50 (d, 1H), 3.62 (s, 2H), 3.20-3.50 (m, 5H); MS (EI) for C₂₁H₁₄BrClF₃N₅O₃, found 558 (MH+).

The following compounds were prepared using the same or analogous synthetic techniques in Example 5 and substituting with appropriate reagents, which were commercially available or were prepared using procedures herein or procedures known to one of ordinary skill in the art.

1-[(4-{5-[8-Chloro-6-(trifluoromethyl)imidazo[1,2-a]pyridin-2-yl]-1,2,4-oxadiazol-3-yl}phenyl)methyl]azetidine-3-carboxylic acid. MS (EI) for C₂₁H₁₅ClF₃N₅O₃, found 478 (MH+).

N-[(4-{5-[8-Chloro-6-(trifluoromethypimidazo[1,2-a]pyridin-2-yl]-1,2,4-oxadiazol-3-yl}phenyl)methyl]-beta-alanine MS (EI) for C₂₀H₁₅ClF₃N₅O₃, found 466.1 (MH+).

1-[(3-Chloro-4-{5-[8-chloro-6-(trifluoromethyl)imidazo[1,2-a]pyridin-2-yl]-1,2,4-oxadiazol-3-yl}phenyl)methyl]azetidine-3-carboxylic acid. ¹H NMR (400 MHz, DMSO-d₆) δ 9.32 (s, 1H), 9.04 (s, 1H), 8.08 (s, 1H), 7.98 (d, 1H), 7.58 (s, 1H); 7.47 (d, 1H), 4.10 (m, 2H), 3.65 (2, 2H), 3.20 (m, 3H).

1-[(2-Chloro-4-{5-[8-chloro-6-(trifluoromethyl)imidazo[1,2-a]pyridin-2-yl]-1,2,4-oxadiazol-3-yl}phenyl)methyl]azetidine-3-carboxylic acid. MS (EI) for C₂₁H₁₄Cl₂F₃N₅O₃, found 512 (MH+).

N-[(3-Chloro-4-{5-[8-chloro-6-(trifluoromethyl)imidazo[1,2-a]pyridin-2-yl]-1,2,4-oxadiazol-3-yl}phenyl)methyl]glycine. MS (EI) for C₁₉H₁₂Cl₂F₃N₅O₃, found 486 (MH+).

1-[(3-{5-[8-Chloro-6-(trifluoromethyl)imidazo[1,2-a]pyridin-2-yl]-1,2,4-oxadiazol-3-yl}phenyl)methyl]azetidine-3-carboxylic acid. MS (EI) for C₂₁H₁₅ClF₃N₅O₃, found 478 (MH+).

N-[(3-Chloro-4-{5-[8-chloro-6-(trifluoromethyl)imidazo[1,2-a]pyridin-2-yl]-1,2,4-oxadiazol-3-yl}phenyl)methyl]-beta-alanine MS (EI) for C₂₀H₁₄Cl₂F₃N₅O₃, found 522 (MNa+).

N-[(4-{5-[8-Bromo-6-(trifluoromethyl)imidazo[1,2-a]pyridin-2-yl]-1,2,4-oxadiazol-3-yl}-3-chlorophenyl)methyl]-beta-alanine ¹H-NMR (400 MHz, DMSO-d6): δ 9.33 (s, 1H), 9.06 (s, 1H) 8.08 (s, 1H), 7.99 (m, 1H), 7.72 (s, 1H), 7.54 (m, 1H), 3.86 (s, 2H), 2.74 (t, 2H), 2.38 (t, 2H).

N-[(3-Chloro-4-{5-[8-chloro-6-(trifluoromethyl)imidazo[1,2-a]pyridin-2-yl]-1,2,4-oxadiazol-3-yl}phenyl)methyl]methanesulfonamide (prepared by treating the amine intermediate, which itself is prepared according to Example 5, with methanesulfonyl chloride). MS (EI) for C₁₈H₁₂Cl₂F₃N₅O₃S, found 507 (MH+).

2-{[(2,5-Dichloro-4-{5-[8-chloro-6-(trifluoromethyl)imidazo[1,2-a]pyridin-2-yl]-1,2,4-oxadiazol-3-yl}phenyl)methyl]amino}ethanol. MS (EI) for C₁₉H₁₃Cl₃F₃N₅O₂, found 506 (MH+).

### Example 6

### 3-(3-Chloro-4-(5-(8-chloro-6-(trifluoromethyl)imidazo[1,2-a]pyridin-2-yl)-1,2,4-oxadiazol-3-yl)phenyl)-3-hydroxypropanoic acid

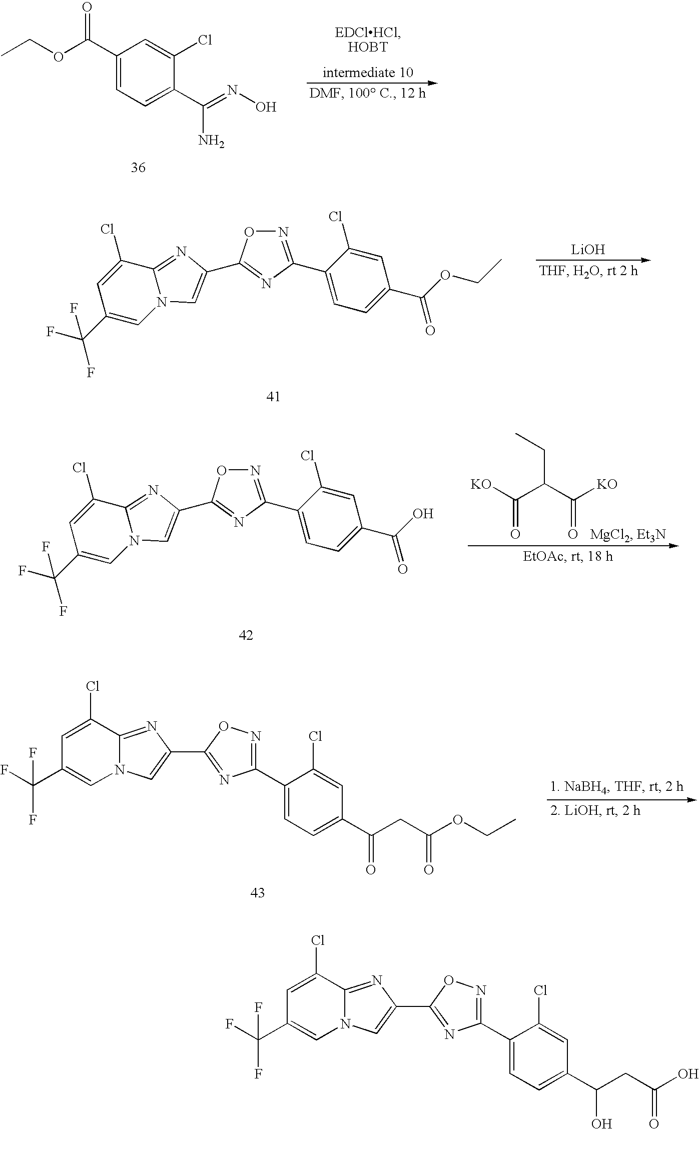

Ethyl 3-chloro-4-(5-(8-chloro-6-(trifluoromethyl)imidazo[1,2-a]pyridin-2-yl)-1,2,4-oxadiazol-3-yl)benzoate (41). To a stirred solution of intermediate 10 (7.8 g, 29 mmol) in DMF (50 mL) was added EDCI.HCl (5.65 g, 29.5 mmol) and HOBT (4.0 g, 29 mmol) at room temperature and stirred for 20 min. Intermediate 36 (5.5 g, 23 mmol) was added and the reaction mixture was stirred at 100° C. for 12 h. After completion, the reaction mixture was concentrated in vacuo and the residue was diluted with EtOAc. The organic phase was washed with saturated sodium bicarbonate solution, water, dried over Na₂SO₄, and concentrated. The crude compound was purified by column chromatography to afford the title intermediate 41 as a white solid (8.0 g, yield 75%).

3-Chloro-4-(5-(8-chloro-6-(trifluoromethyl)imidazo[1,2-a]pyridin-2-yl)-1,2,4-oxadiazol-3-yl)benzoic acid (42). To a stirred solution of ester 41 (8.0 g, 17 mmol) in 1:1 THF:water (100 mL) was added lithium hydroxide (2.13 g, 50.9 mmol) and the reaction was stirred at room temperature for 2 h. After completion, THF was removed in vacuo and the aqueous phase was acidified to pH 5 with 1 N HCl and the product extracted into EtOAc. The organic phase was dried over Na₂SO₄ and concentrated in vacuo. The crude intermediate was purified by column chromatography to afford the title intermediate 42 as a white solid (6.0 g, yield 80%).

Ethyl 3-(3-chloro-4-(5-(8-chloro-6-(trifluoromethyl)imidazo[1,2-a]pyridin-2-yl)-1,2,4-oxadiazol-3-yl)phenyl)-3-oxopropanoate (43). To a stirred solution of potassium ethyl malonate (2.58 g, 15.2 mmol) in EtOAc (80 mL) was added triethylamine (5.28 g, 52.3 mmol) at 0° C. The resulting mixture was stirred at 0-5° C. for 18 h. Meanwhile, to a stirred suspension of acid 42 (4.0 g 9.0 mmol) in DCM was added oxalyl chloride (5.72 g, 45.1 mmol) and DMF (1 drop). The reaction was stirred at 0-5° C. for 1 h. After completion, the reaction mixture was concentrated, the resulting mixture was dissolved in EtOAc and the solution was added dropwise to the previously prepared solution under ice cooling. After addition was complete, the reaction was allowed to warm to room temperature and stirred for 18 h. After completion, 10% citric acid was added dropwise and stirred for 30 min. The organic phase was separated, dried over Na₂SO₄ and concentrated in vacuo. The crude intermediate was purified by column chromatography to afford the title intermediate 43 as a white solid (1.2 g, yield 26%).

3-(3-Chloro-4-(5-(8-chloro-6-(trifluoromethyl)imidazo[1,2-a]pyridin-2-yl)-1,2,4-oxadiazol-3-yl)phenyl)-3-hydroxypropanoic acid. To a stirred solution of intermediate 43 (0.2 g, 0.4 mmol) in THF (5 mL) was added sodium borohydride (0.017 g, 0.47 mmol) at 0° C. After addition, the reaction was allowed to warm to room temperature and stirred for 2 h. After completion, the reaction mixture was quenched with water, followed by the addition of lithium hydroxide (0.048 g, 1.2 mmol) with stirring at room temperature for 2 h. After completion, solvent was removed in vacuo and the reaction mixture was acidified to pH 4 with acetic acid. The aqueous phase was extracted with EtOAc and the combined organic layers were dried over Na₂SO₄ and concentrated under reduced pressure. The crude compound was purified by preparative HPLC to afford the title compound as a white solid (0.178 g, yield 94.2%). ¹H NMR (400 MHz, DMSO-d₆) δ 12.21 (br s, 1H), 9.29 (s, 1H), 9.01 (s, 1H), 8.2 (s, 1H), 8.0 (d, 1H), 7.65 (s, 1H), 7.58 (d, 1H), 5.80 (s, 1H), 5.0 (s, 1H), 2.65 (d, 2H); MS (EI) for C₁₉H₁₁Cl₂F₃N₄O₄, found 487 (MH+).

Using same or analogous synthetic techniques in Example 6 and substituting with appropriate reagents (which were commercially available or prepared using procedures known to one of ordinary skill in the art), 1-(3-Chloro-4-{5-[8-chloro-6-(trifluoromethyl)imidazo[1,2-a]pyridin-2-yl]-1,2,4-oxadiazol-3-yl}phenyl)propane-1,3-diol was prepared. MS (EI) for C₁₉H₁₃Cl₂F₃N₄O₃, found 473 (MH+).

### Example 7

### 3-(2-Chloro-4-(methylsulfonylmethyl)phenyl)-5-(8-chloro-6-(trifluoromethyl)imidazo[1,2-a]pyridin-2-yl)-1,2,4-oxadiazole

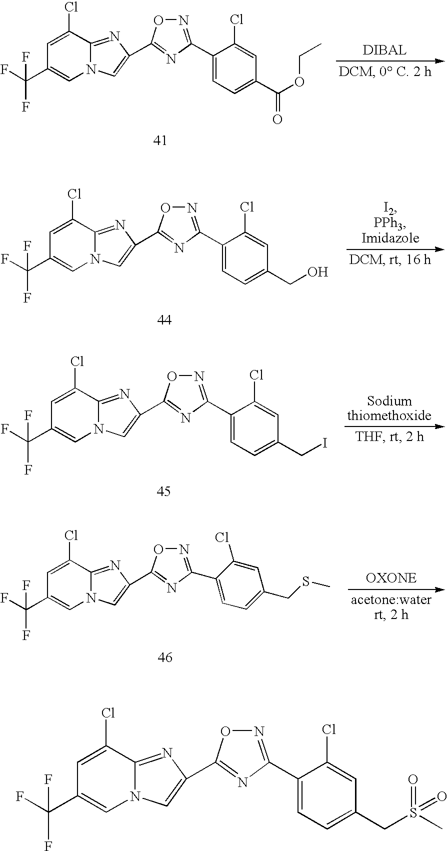

(3-Chloro-4-(5-(8-chloro-6-(trifluoromethyl)imidazo[1,2-a]pyridin-2-yl)-1,2,4-oxadiazol-3-yl)phenyl)methanol (44). To a stirred solution of intermediate 41 (6.0 g, 13 mmol), prepared using conditions described in Example 6, in DCM (50 mL) was added DIBAL (7.89 g, 63.6 mmol) dropwise at 0° C. and stirred at 0° C. for 2 h. After completion, the reaction mixture was quenched with EtOAc followed by saturated ammonium chloride solution at −40° C. and extracted with EtOAc. The organic phase was washed with water, saturated NaCl, dried over Na₂SO₄ and concentrated. The resulting solid was washed with iPrOH to afford the title intermediate 44 as a light yellow solid (3.8 g, yield 70%).

3-(2-Chloro-4-(iodomethyl)phenyl)-5-(8-chloro-6-(trifluoromethyl)imidazo[1,2-a]pyridin-2-yl)-1,2,4-oxadiazole (45). To a stirred solution of triphenylphosphine (2.05 g, 7.81 mmol) in DCM (30 mL) was added imidazole (0.533 g, 7.81 mmol) followed by iodine (1.98 g, 7.81 mmol). After stirring for 15 min, compound 44 (2.8 g, 6.5 mol) was added and the reaction was stirred at room temperature for 16 h. After completion, the reaction was concentrated in vacuo. The crude compound was purified by column chromatography, eluting with 30% EtOAc/hexane, to afford the title intermediate 45 as a white solid (2.6 g, yield 74%).

3-(2-Chloro-4-(methylthiomethyl)phenyl)-5-(8-chloro-6-(trifluoromethyl)imidazo[1,2-a]pyridin-2-yl)-1,2,4-oxadiazole (46). To a stirred solution of compound 45 (0.370 g, 0.68 mmol) in THF (6 mL) was added sodium thiomethoxide (0.048 g, 0.68 mmol) at 0° C. and the reaction mixture was stirred at room temperature for 2 h. After completion, reaction was quenched with water and extracted with EtOAc. The combined organic layers were dried over Na₂SO₄ and concentrated under reduced pressure to afford the title compound 46 as a white solid (0.287 g, yield 91.1%).

3-(2-Chloro-4-(methylsulfonylmethyl)phenyl)-5-(8-chloro-6-(trifluoromethyl)imidazo[1,2-a]pyridin-2-yl)-1,2,4-oxadiazole. To a stirred solution of intermediate 46 (0.700 g, 1.52 mmol) in a mixture of acetone (35 mL) and water (7 mL) was added Oxone (0.878 g, 1.42 mmol) at 0° C. The reaction mixture was stirred at room temperature for 2 h. After completion, the reaction mixture was concentrated and diluted with water. The suspended solid was collected by filtration, washed well with water and acetone and dried completely to afford the title compound as a white solid (0.140 g, yield 18.7%). ¹H NMR (400 MHz, DMSO-d₆) δ 9.30 (s, 1H), 9.05 (s, 1H), 8.12 (m, 2H), 7.80 (s, 1H), 7.62(d, 1H), 4.75 (s, 2H), 3.0 (s, 3H); MS (EI) for C₁₈H₁₁Cl₂F₃N₄O₃S, found 491 (MH+).

### Example 8

### 5-(8-Chloro-6-(trifluoromethyl)imidazo[1,2-a]pyridin-2-yl)-3-(1H-indol-4-yl)-1,2,4-oxadiazole

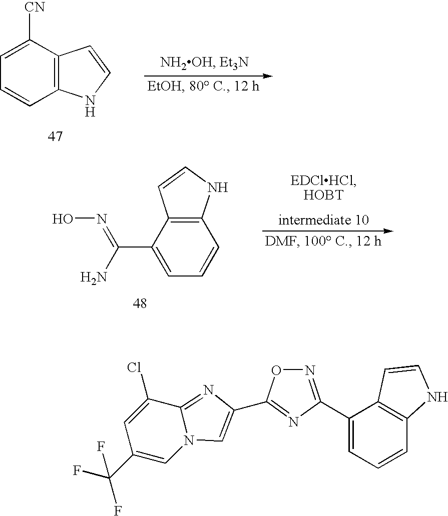

N′-Hydroxy-1H-indole-4-carboximidamide (48). To a stirred solution of hydroxylamine hydrochloride (8.79 g, 127 mmol) in EtOH (60 mL) was added triethylamine (14.95 g, 147.7 mmol) and the resulting mixture stirred at room temperature for 30 min. Intermediate 47 (3.00 g, 21.1 mmol) was added and the resulting mixture was stirred at 80° C. for 12 h. After completion, the reaction mixture was concentrated in vacuo and diluted with EtOAc (100 mL). The organic layer was washed with water (2×50 mL), and saturated NaCl solution. The organic layers were collected and dried over Na₂SO₄ and concentrated to afford hydroxyimidate 48 as a white solid (2.58 g, yield 70%).

5-(8-Chloro-6-(trifluoromethyl)imidazo[1,2-a]pyridin-2-yl)-3-(1H-indol-4-yl)-1,2,4-oxadiazole. To a stirred solution of Intermediate 10 (5.84 g, 22.1 mmol) in DMF (51 mL) was added EDCI.HCl (4.23 g, 22.1 mmol) followed by HOBT (2.98 g, 22.1 mmol) and hydroxyimidate 48 (2.58 g, 14.7 mmol). The resulting mixture was stirred at room temperature for 1 h and then at 100° C. for 12 h. After completion, the reaction mixture was concentrated in vacuo and diluted with EtOAc. The organic layer was washed with saturated sodium bicarbonate and water. The organic layers were collected, dried over Na₂SO₄, and concentrated. The residue was purified by column chromatography (EtOAc/hexane as eluent) to afford the title compound as a yellow solid (3.56 g, yield 60%). ¹H NMR (400 MHz, DMSO-d₆) δ 11.58 (s, 1H), 9.29 (s, 1H) 9.12 (s,1H), 8.12 (s, 1H), 7.85 (d, 1H), 7.65 (d, 1H), 7.60 (s, 1H), 7.25 (t, 1H), 7.15 (s, 1H); MS (EI) for C₁₈H₉ClF₃N₅O, found 404 (MH+).

The following compounds were prepared using the same or analogous synthetic techniques in Example 8 and substituting with appropriate reagents, which were commercially available or were prepared using procedures herein or procedures known to one of ordinary skill in the art.

8-Chloro-2-[3-(2,6-difluorophenyl)-1,2,4-oxadiazol-5-yl]-6-(trifluoromethyl)imidazo[1,2-a]pyridine. MS (EI) for, found 400.8 (MH+).

8-Chloro-2-[3-(2-fluorophenyl)-1,2,4-oxadiazol-5-yl]-6-(trifluoromethypimidazo[1,2-a]pyridine. MS (EI) for C₁₆H₇ClF₄N₄O, found 382.9 (MH+).

8-Chloro-2-[3-(2-chlorophenyl)-1,2,4-oxadiazol-5-yl]-6-(trifluoromethyl)imidazo[1,2-a]pyridine. MS (EI) for C₁₆H₇Cl₂F₃N₄O, found 398.9 (MH+).

8-Chloro-6-(trifluoromethyl)-2-{3-[3-(trifluoromethyl)phenyl]-1,2,4-oxadiazol-5-yl}imidazo[1,2-a]pyridine. MS (EI) for C₁₇H₇ClF₆N₄O, found 432.9 (MH+).

8-Chloro-2-[3-(2,4-difluorophenyl)-1,2,4-oxadiazol-5-yl]-6-(trifluoromethyl)imidazo[1,2-a]pyridine. MS (EI) for C₁₆H₆ClF₅N₄O, found 401 (MH+).

8-Chloro-2-{3-[2-chloro-4-(trifluoromethyl)phenyl]-1,2,4-oxadiazol-5-yl}-6-(trifluoromethyl)imidazo[1,2-a]pyridine. MS (EI) for C₁₇H₆Cl₂F₆N₄O, found 467 (MH+).

8-Chloro-2-[3-(2-chloro-6-fluorophenyl)-1,2,4-oxadiazol-5-yl]-6-(trifluoromethyl)imidazo[1,2-a]pyridine. MS (EI) for C₁₆H₆Cl₂F₄N₄O, found 417 (MH+).

8-Chloro-2-[3-(2-methylphenyl)-1,2,4-oxadiazol-5-yl]-6-(trifluoromethyl)imidazo[1,2-a]pyridine. MS (EI) for C₁₇H₁₀ClF₃N₄O, found 379 (MH+).

8-Chloro-2-[3-(4-fluoro-2-methylphenyl)-1,2,4-oxadiazol-5-yl]-6-(trifluoromethyl)imidazo[1,2-a]pyridine. ¹H-NMR (400 MHz, CDCl₃) δ 8.54 (d, 2H), 8.16 (t, 1H), 7.98 (d, 1H) 7.57 (s, 1H), 7.04 (m, 2H), 2.70 (s, 3H); MS (EI) for C₁₇H₉ClF₄N₄O, found 397 (MH+).

8-Chloro-2-[3-(2-chloro-3-fluorophenyl)-1,2,4-oxadiazol-5-yl]-6-(trifluoromethyl)imidazo[1,2-a]pyridine. MS (EI) for C₁₆H₆Cl₂F₄N₄O, found 417 (MH+).

2-[3-(2-Bromo-4-fluorophenyl)-1,2,4-oxadiazol-5-yl]-8-chloro-6-(trifluoromethyl)imidazo[1,2-a]pyridine. MS (EI) for C₁₆H₆BrClF₄N₄O, found 461 (MH+).

8-Bromo-2-[3-(2-chlorophenyl)-1,2,4-oxadiazol-5-yl]-6-(trifluoromethyl)imidazo[1,2-a]pyridine. MS (EI) for C₁₆H₇BrClF₃N₄O, found 443 (MH+).

8-Chloro-2-[3-(1H-indol-5-yl)-1,2,4-oxadiazol-5-yl]-6-(trifluoromethyl)imidazo[1,2-a]pyridine. MS (EI) for C₁₈H₉ClF₃N₅O, found 404 (MH+).

5-{5-[8-Chloro-6-(trifluoromethyl)imidazo[1,2-a]pyridin-2-yl]-1,2,4-oxadiazol-3-yl}-1H-benzimidazole. ¹H-NMR (400 MHz, DMSO-d₆) δ 12.80 (s, 1H), 9.28 (s, 1H), 9.03 (s, 1H) 8.28 (d, 2H), 8.00 (d, 2H), 7.80 (m, 2H); MS (EI) for C₁₇H₈ClF₃N₆O, found 405 (MH+).

5-{5-[8-Chloro-6-(trifluoromethyl)imidazo[1,2-a]pyridin-2-yl]-1,2,4-oxadiazol-3-yl}-1H-indole-2-carboxylic acid. MS (EI) for C₁₉H₉ClF₃N₅O₃, found 448 (MH+).

5-{5-[8-Chloro-6-(trifluoromethyl)imidazo[1,2-a]pyridin-2-yl]-1,2,4-oxadiazol-3-yl}-1-benzofuran-2-carboxylic acid. MS (EI) for C₁₉H₈ClF₃N₄O₄, found 449 (MH+).

8-Chloro-2-[3-(2-chloro-4-fluorophenyl)-1,2,4-oxadiazol-5-yl]-6-(trifluoromethyl)imidazo[1,2-a]pyridine. ¹H NMR (400 MHz, CDCl₃) δ 8.59-8.57 (m, 2H), 8.56 (s, 1H), 8.11 (dd, J=8.8, 6.0, 2H), 7.57 (d, J=1.5, 2H), 7.32 (dt, J=8.3, 2.5, 2H), 7.27 (s, 1H), 7.15 (ddd, J=8.8, 7.7, 2.6, 2H); MS (EI) for C₁₆H₆Cl₂F₄N₄O, found 417 (MH+).

8-Chloro-6-(trifluoromethyl)-2-{3-[2-(trifluoromethyl)phenyl]-1,2,4-oxadiazol-5-yl}imidazo[1,2-a]pyridine. ¹H NMR (400 MHz, CDCl₃) δ 8.59-8.57 (m, 1H), 8.56 (s, 1H), 7.92-7.84 (m, 2H), 7.73-7.64 (m, 2H), 7.56 (d, J=1.5, 1H); MS (EI) for C₁₇H₇ClF₆N₄O, found 433 (MH+).

8-Chloro-6-(trifluoromethyl)-2-{3-[4-(trifluoromethyl)pyridin-3-yl]-1,2,4-oxadiazol-5-yl}imidazo[1,2-a]pyridine. ¹H NMR (400 MHz, CDCl₃) δ 9.24 (s, 1H), 8.99 (d, J=5.1, 1H), 8.58 (d, J=2.4, 2H), 7.76 (d, J=5.2, 1H), 7.58 (d, J=1.4, 1H); MS (EI) for C₁₆H₆ClF₆N₅O, found 434 (MH+).

8-Bromo-2-[3-(2-chloro-4-fluorophenyl)-1,2,4-oxadiazol-5-yl]-6-(trifluoromethyl)imidazo[1,2-a]pyridine. ¹H NMR (400 MHz, CDCl₃) δ 8.56-8.51 (m, 1H), 8.45 (d, J=3.5, 1H), 8.27 (dd, J=8.8, 5.9, 1H), 7.52 (d, J=1.5, 1H), 7.35 (dd, J=8.4, 2.5, 1H), 7.19 (ddd, J=8.9, 7.5, 2.5, 1H); MS (EI) for C₁₆H₆BrClF₄N₄O, found 462 (MH+).

2-[3-(2-Chloro-4-fluorophenyl)-1,2,4-oxadiazol-5-yl]-6-(trifluoromethyl)imidazo[1,2-a]pyridine-8-carbonitrile. ¹H NMR (400 MHz, DMSO-d₆) δ 9.62 (d, J=1.3, 1H), 9.09 (s, 1H), 8.69 (d, J=1.7, 1H), 8.13 (dd, J=8.8, 6.2, 1H), 7.81-7.73 (m, 1H), 7.50 (td, J=8.4, 2.6, 1H); MS (EI) for C₁₇H₆ClF₄N₅O, found 408 (MH+).

8-Bromo-2-[3-(2-chloro-6-fluorophenyl)-1,2,4-oxadiazol-5-yl]-6-(trifluoromethyl)imidazo[1,2-a]pyridine. ¹H NMR (400 MHz, CDCl₃) δ 8.62-8.59 (m, 3H), 8.58 (s, 3H), 7.75 (d, J=1.5, 3H), 7.47 (td, J=8.3, 5.8, 3H), 7.37 (dd, J=8.2, 0.9, 3H), 7.19 (d, J=1.0, 1H); MS (EI) for C₁₆H₆BrClF₄N₄O, found 462 (MH+).

### Example 9

### 2-(4-(5-(8-Chloro-6-(trifluoromethyl)imidazo[1,2-a]pyridin-2-yl)-1,2,4-oxadiazol-3-yl)-1H-indol-1-yl)-N-(2-hydroxyethyl)acetamide

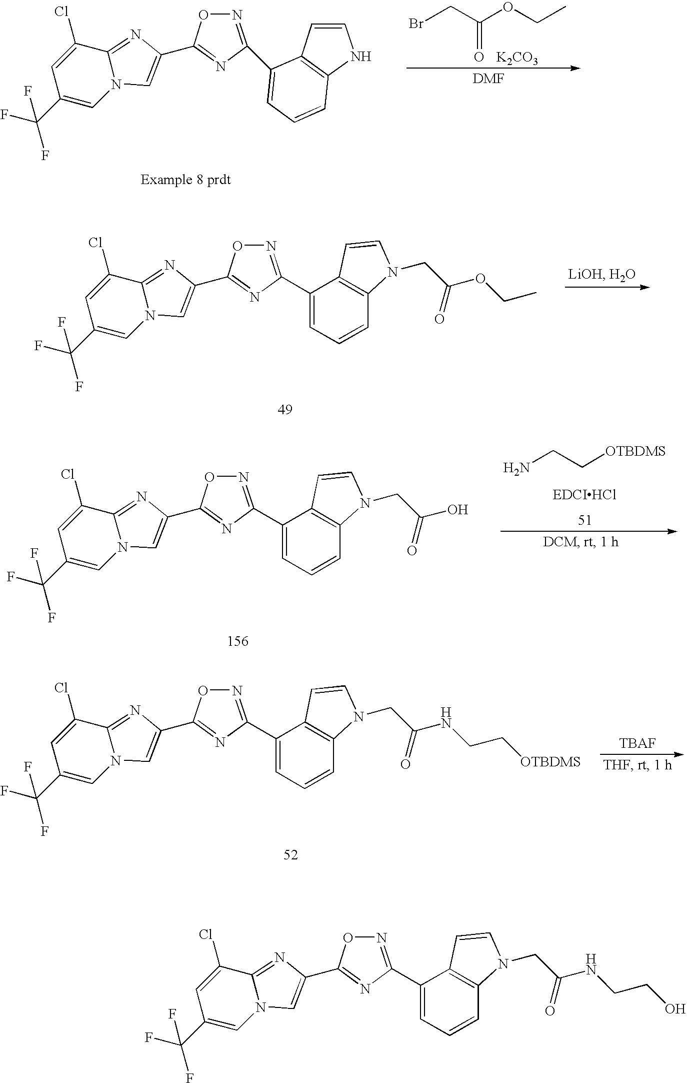

Ethyl [2-(4-(5-(8-chloro-6-(trifluoromethyl)imidazo[1,2-a]pyridin-2-yl)-1,2,4-oxadiazol-3-yl)-1H-indol-1-yl)]acetate (49). To a stirred solution of 5-(8-Chloro-6-(trifluoromethyl)imidazo[1,2-a]pyridin-2-yl)-3-(1H-indol-4-yl)-1,2,4-oxadiazole (3.00 g, 7.43 mmol), prepared as described in Example 8, in DMF (60 mL) was added ethyl 2-bromoacetate (1.48 g, 8.91 mmol) and K₂CO₃ (2.053 g, 14.86 mmol) and the resulting mixture was heated at 80° C. for 12 h. After completion, the reaction mixture was concentrated in vacuo and diluted with EtOAc. The organic phase was washed with water, saturated NaCl solution, dried over Na₂SO₄ and concentrated. The crude compound was purified by column chromatography (EtOAc/hexane as eluent) to afford the title intermediate 49 as a white solid (2.10 g, yield 58%).

2-(4-(5-(8-Chloro-6-(trifluoromethyl)imidazo[1,2-a]pyridin-2-yl)-1,2,4-oxadiazol-3-yl)-1H-indol-1-yl)acetic acid. To a stirred solution of ester 49 (2.10 g, 4.28 mmol) in 20 mL THF:water (1:1) was added lithium hydroxide (0.205 g, 8.57 mmol) and the resulting mixture stirred at room temperature for 3 h. After completion, solvent was removed in vacuo and the aqueous phase was acidified by dropwise addition of acetic acid at 0° C. The product was extracted into EtOAc and the organic layers combined, dried over Na₂SO₄, and concentrated to afford the title compound 156 as a white solid (1.58 g, yield 80%). MS (EI) for C₂₀H₁₁ClF₃N₅O₃, found 461.9 (MH+).

N-(2-(tert-Butyldimethylsilyloxy)ethyl)-2-(4-(5-(8-chloro-6-trifluoromethyl)imidazo[1,2-a]pyridin-2-yl)-1,2,4-oxadiazol-3-yl)-1H-indol-1-yl)acetamide (52). To a stirred solution of intermediate 156 (0.220 g, 0.476 mmol) in DCM (10 mL) was added EDCI.HCl (0.090 g, 0.47 mmol) followed by amine 51 (0.167 g, 0.952 mmol) and the resulting mixture was stirred at room temperature for 1 h. After completion, the reaction mixture was concentrated and purified by column chromatography (50% EtOAc/hexane as eluent) to afford the title intermediate 52 as a pale yellow solid (0.20 g, yield 68%).

2-(4-(5-(8-Chloro-6-(trifluoromethyl)imidazo[1,2-a]pyridin-2-yl)-1,2,4-oxadiazol-3-yl)-1H-indol-1-yl)-N-(2-hydroxyethyl)acetamide. To a stirred solution of intermediate 52 (0.45 g, 0.72 mmol) in THF (10 mL) was added tetrabutylammonium chloride (0.52 g, 0.5 mL, 1.9 mmol) dropwise at 0° C. After addition, the reaction mixture was allowed to warm to room temperature and stirred for 1 h. After completion, the reaction mixture was cooled to 0° C. and quenched with saturated ammonium chloride and extracted with EtOAc. The organic layer was washed with saturated NaCl, dried over Na₂SO₄ and concentrated. The crude compound was stirred in iPrOH and the suspended solid was filtered, washed well with iPrOH and ether and dried completely to afford the title compound as a white solid (0.128 g, yield 35%). ¹H NMR (400 MHz, DMSO-d₆) δ 9.29 (s, 1H), 9.15 (s, 1H), 8.22 (t, 1H), 8.12 (s, 1H), 8.0 (d, 1H), 7.63 (d, 1H), 7.60 (s, 1H), 7.40 (t, 1H), 7.13 (s, 1H), 4.90 (s, 2H) 7.65 (t, 1H), 3.43 (m, 2H), 3.19 (m, 2H); MS (EI) for C₂₂H₁₆ClF₃N₆O₃, found 505 (MH+).

Using the same or analogous synthetic techniques in Example 9, and substituting with appropriate reagents (which were commercially available or were prepared using procedures known to one of ordinary skill in the art), the following compound was prepared. 2-(4-{5-[8-chloro-6-(trifluoromethyl)imidazo[1,2-a]pyridin-2-yl]-1,2,4-oxadiazol-3-yl}-1H-indol-1-yl)acetamide. MS (EI) for C₂₀H₁₂ClF₃N₆O₂, found 461 (MH+).

### Example 10

### 3-(4-(5-(8-Bromo-6-(trifluoromethyl)imidazo[1,2-a]pyridin-2-yl)-1,2,4-oxadiazol-3-yl)-3-methylphenyl)butanoic acid

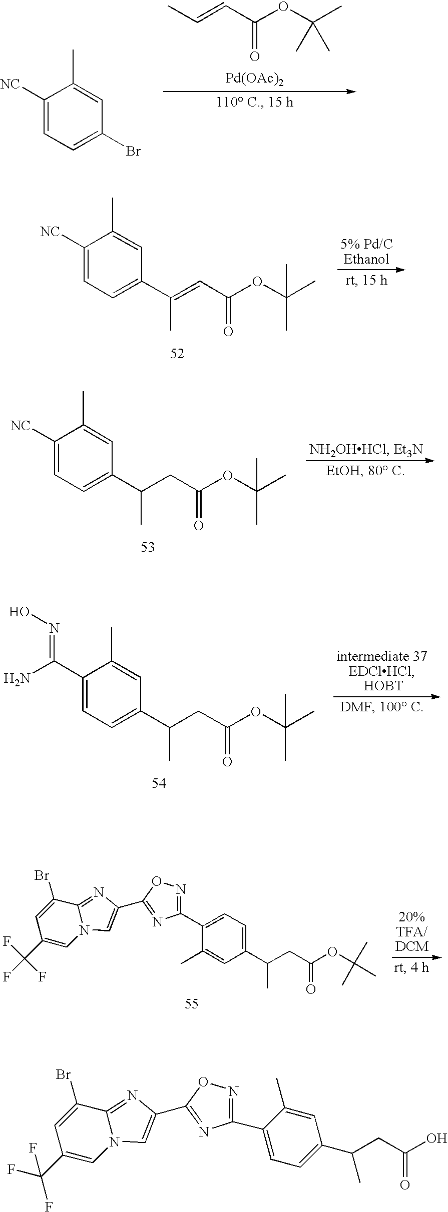

tert-Butyl 3-(4-cyano-3-methylphenyl)but-2-enoate (52). To a stirred solution of 4-bromo-2-methylbenzonitrile (5.0 g, 26 mmol) in dimethylacetamide (50 mL) was added tent-butyl crotonate (4.35 g, 30.6 mmol) and the reaction mixture was degassed with argon. To this solution, Pd(OAc)₂ (0.114 g, 0.510 mmol) was added followed by tetraethylammonium chloride (4.22 g, 25.5 mmol) and the reaction was stirred at 100° C. for 15 h. After completion, the reaction mixture was quenched with ice cold water and extracted with ether. The combined organic layers were washed with water, saturated NaCl solution, dried over Na₂SO₄ and concentrated in vacuo. The crude compound was purified by column chromatography to afford the title intermediate 52 as a yellow solid (5.2 g, yield 80%).

tert-Butyl 3-(4-cyano-3-methylphenyl)butanoate (53). To a stirred solution of intermediate 52 (5.2 g, 20.2 mmol) in EtOH (50 mL) was added 5% Pd/C (0.52 g) and the reaction was stirred under hydrogen atmosphere for 12 h. After completion, the reaction mixture was filtered and the filtrate was concentrated in vacuo to afford the title intermediate 53 (4.4 g, yield 85%).

tert-Butyl 3-(4-(N′-hydroxycarbamimidoyl)-3-methylphenyl)butanoate (54). To a stirred solution of hydroxylamine hydrochloride (7.0 g, 102 mmol) in EtOH (80 mL) was added triethylamine (12 g, 120 mmol) and the reaction was stirred at room temperature for 30 min. Intermediate 53 (4.4 g, 17 mmol) was then added and the reaction was stirred at 80° C. for 12 h. After completion, the reaction mixture was concentrated in vacuo and diluted with EtOAc. The organic phase was washed with water, saturated NaCl solution, dried over Na₂SO₄ and concentrated to afford the title intermediate 54 as a white solid (4.0 g, yield 82%).

tert-Butyl [3-(4-(5-(8-bromo-6-(trifluoromethyl)imidazo[1,2-a]pyridin-2-yl)-1,2,4-oxadiazol-3-yl)-3-methylphenyl)]butanoate (55). To a stirred solution of acid 37 (6.3 g, 21 mmol) in DMF (80 mL) was added EDCI.HCl (3.93 g, 20.5 mmol) followed by addition of HOBT (2.77 g, 20.5 mmol) and hydroxyimidate 54 (4.0 g, 14 mmol). The reaction was stirred at room temperature for 1 h and then at 100° C. for 12 h. After completion, the reaction mixture was concentrated in vacuo and diluted with EtOAc. The organic phase was washed with saturated sodium bicarbonate, water, saturated NaCl solution, dried over Na₂SO₄ and concentrated. The crude compound was purified by column chromatography (10% EtOAc/hexane as eluent) to afford the title intermediate 55 as a yellow solid (4.6 g, yield 60%).

3-(4-(5-(8-Bromo-6-(trifluoromethyl)imidazo[1,2-a]pyridin-2-yl)-1,2,4-oxadiazol-3-yl)-3-methylphenyl)butanoic acid. A solution of intermediate 55 (0.250 g, 0.44 mmol) in 20% TFA/DCM (50 mL) was stirred at room temperature for 4 h. After completion, the reaction mixture was concentrated in vacuo. The residue obtained was triturated with diethyl ether and further washed with iPrOH to afford the title compound as a white solid (0.120 g, yield 54%). ¹H NMR (400 MHz, DMSO-d₆) δ 12.17 (s, 1H), 9.39 (s, 1H), 9.0 (s, 1H), 8.19 (s, 1H), 8.0 (d, 1H), 7.39 (s, 1H), 7.30 (d, 1H), 3.20 (q, 1H), 2.62 (s, 1H), 2.60 (d, 2H), 1.23 (d, 2H); MS (EI) for C₂₁H₁₆BrF₃N₄O₃, found 509 (MH+).

The following compounds were prepared using the same or analogous synthetic techniques in Example 10 and substituting with appropriate reagents, which were commercially available or were prepared using procedures herein or procedures known to one of ordinary skill in the art.

3-(5-Chloro-4-{5-[8-chloro-6-(trifluoromethyl)imidazo[1,2-a]pyridin-2-yl]-1,2,4-oxadiazol-3-yl}-2-fluorophenyl)-2-methylpropanoic acid. MS (EI) for C₂₀H₁₂Cl₂F₄N₄O₃, found 503 (MH+).

3-(3-Chloro-4-{5-[8-chloro-6-(trifluoromethyl)imidazo[1,2-a]pyridin-2-yl]-1,2,4-oxadiazol-3-yl}phenyl)butanoic acid. MS (EI) for C₂₀H₁₃Cl₂F₃N₄O₃, found 485 (MH+).

3-(4-{5-[8-Chloro-6-(trifluoromethyl)imidazo[1,2-a]pyridin-2-yl]-1,2,4-oxadiazol-3-yl}-3-methylphenyl)-2-methylpropanoic acid. MS (EI) for C₂₁H₁₆ClF₃N₄O₃, found 465 (MH+).

3-(4-{5-[8-Bromo-6-(trifluoromethyl)imidazo[1,2-a]pyridin-2-yl]-1,2,4-oxadiazol-3-yl}-3-methylphenyl)-2-methylpropanoic acid. MS (EI) for C₂₁H₁₆BrF₃N₄O₃, found 509 (MH+).

3-(3-Chloro-4-{5-[8-chloro-6-(trifluoromethyl)imidazo[1,2-a]pyridin-2-yl]-1,2,4-oxadiazol-3-yl}phenyl)butanamide (prepared from the acid intermediate, which itself was prepared using procedures analogous to those in Example 10, by treatment with NH₃ using procedures known to one of skill in the art). MS (EI) for C₂₀H₁₄Cl₂F₃N₅O₂, found 484 (MH+).

3-(3-Chloro-4-{5-[8-chloro-6-(trifluoromethyl)imidazo[1,2-a]pyridin-2-yl]-1,2,4-oxadiazol-3-yl}phenyl)-2-methylpropanoic acid. ¹H-NMR (400 MHz, DMSO-d₆) δ 12.29 (s, 1H), 9.37 (s, 1H), 9.05 (s, 1H) 8.05 (s, 1H), 7.95 (d, 1H), 7.58 (s, 2H), 7.40 (s, 1H), 3.01 (m, 2H), 2.78 (m, 1H), 1.05 (s, 3H); MS (EI) for C₂₀H₁₃Cl₂F₃N₄O₃, found 485 (MH+).

3-(4-{5-[8-Chloro-6-(trifluoromethyl)imidazo[1,2-a]pyridin-2-yl]-1,2,4-oxadiazol-3-yl}-3-methylphenyl)butanoic acid. MS (EI) for C₂₁H₁₆ClF₃N₄O₃, found 465 (MH+).

3-(4-{5-[8-Chloro-6-(trifluoromethyl)imidazo[1,2-a]pyridin-2-yl]-1,2,4-oxadiazol-3-yl}-3-fluorophenyl)-2-methylpropanoic acid. MS (EI) for C₂₀H₁₃ClF₄N₄O₃, found 469 (MH+).

3-(4-{5-[8-Bromo-6-(trifluoromethyl)imidazo[1,2-a]pyridin-2-yl]-1,2,4-oxadiazol-3-yl}-3-chlorophenyl)-2-methylpropanoic acid. MS (EI) for C₂₀H₁₃BrClF₃N₄O₃, found 529 (MH+).

3-(4-{5-[8-Chloro-6-(trifluoromethyl)imidazo[1,2-a]pyridin-2-yl]-1,2,4-oxadiazol-3-yl}-3-fluorophenyl)butanoic acid. MS (EI) for C₂₀H₁₃ClF₄N₄O₃, found 469 (MH+).

3-(4-{5-[8-Bromo-6-(trifluoromethyl)imidazo[1,2-a]pyridin-2-yl]-1,2,4-oxadiazol-3-yl}-3-chlorophenyl)butanoic acid. MS (EI) for C₂₀H₁₃BrClF₃N₄O₃, found 529 (MH+).

3-(2,5-Dichloro-4-{5-[8-chloro-6-(trifluoromethyl)imidazo[1,2-a]pyridin-2-yl]-1,2,4-oxadiazol-3-yl}phenyl)-2-methylpropanoic acid. MS (EI) for C₂₀H₁₂Cl₃F₃N₄O₃, found 519 (MH+).

3-(4-{5-[8-Bromo-6-(trifluoromethyl)imidazo[1,2-a]pyridin-2-yl]-1,2,4-oxadiazol-3-yl}-2,5-dichlorophenyl)-2-methylpropanoic acid. ¹H-NMR (400 MHz, DMSO-d₆) δ 12.38 (br s, 1H), 9.35 (s, 1H), 9.08 (s, 1H) 8.15 (s, 1H), 8.08 (s, 1H), 7.71 (s, 1H), 3.10 (m, 1H), 2.82 (m, 2H), 1.12 (d, 3H); MS (EI) for C₂₀H₁₂BrCl₂F₃N₄O₃, found 564 (MH+).

### Example 11

### 3-(4-(5-(8-Bromo-6-(trifluoromethyl)imidazo[1,2-a]pyridin-2-yl)-1,2,4-oxadiazol-3-yl)-2-chloro-5-methylphenyl)propanoic acid

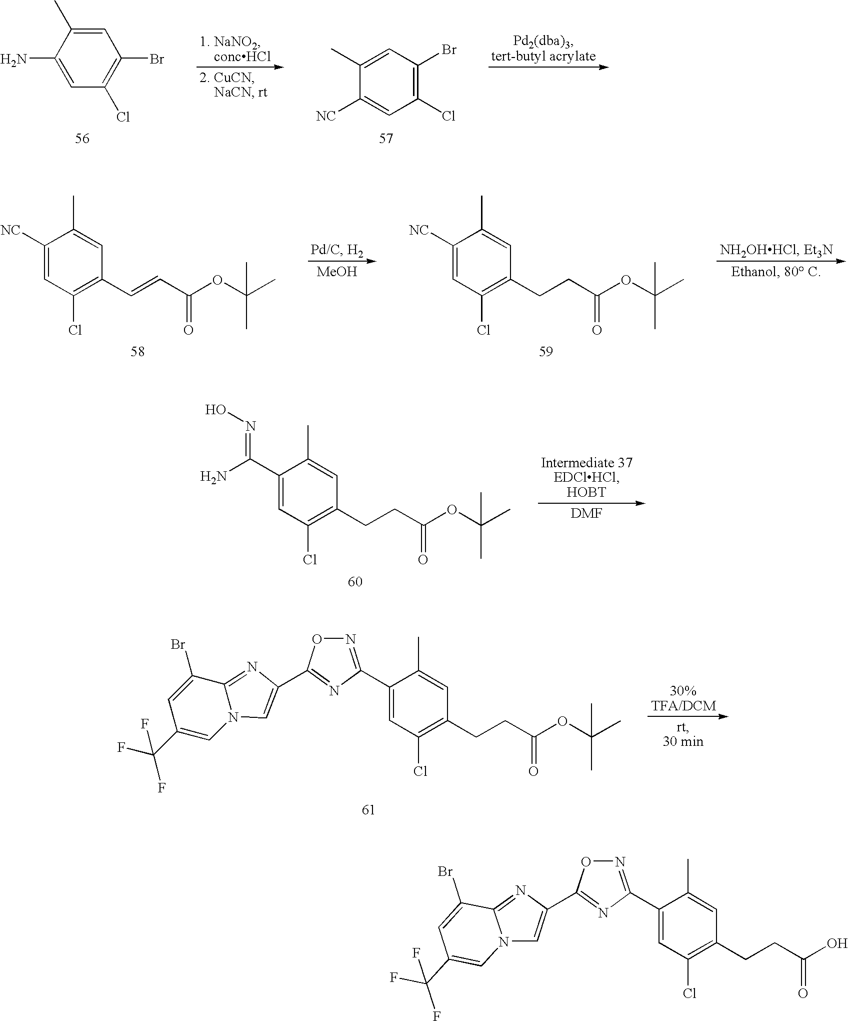

2-Methyl-4-bromo-5-chloro benzonitrile (57). To a stirred solution of 2-methyl-4-bromo-5-chloro aniline (56) (2.9 g, 13 mmol) in concentrated HCl (14.5 mL) cooled to 0° C. was added a solution of sodium nitrite (1 g, 14 mmol) in water (3.5 mL) slowly over 20 min. After stirring at 0° C. for 35 min, a pre-cooled solution of copper (I) cyanide (11.80 g, 13.1 mmol) and sodium cyanide (6.46 g, 13.1 mmol) in water (81 mL) was gradually added to the above solution of diazonium salt at 0° C. over a period of 50 min. After addition, the resulting mixture was stirred at room temperature for 18 h. The precipitated solid was filtered, washed with water and dried. The resulting solid was then dissolved in EtOAc and washed with water followed by saturated NaCl solution. The organic phase was dried over Na₂SO₄ and concentrated in vacuo. The crude compound was purified by column chromatography, eluting with 5-10% EtOAc/hexane, to afford the title intermediate 57 as a white solid (2.1 g, yield 69%).

tert-Butyl 3-(2-chloro-4-cyano-5-methylphenyl)acrylate (58). To a stirred solution of intermediate 57 (2.0 g, 8.7 mmol) in 1,4-dioxane (25 mL) was added tent-butyl acrylate (1.44 g, 11.3 mmol) and the reaction was degassed with argon. To this degassed solution, Pd₂(dba)₃ (76 mg, 0.070 mmol) and (2-biphenyl)di-tert-butylphosphine (38 mg, 0.13 mmol) was added followed by addition of triethylamine (1.75 g, 17.4 mmol). The reaction mixture was then stirred at 100° C. for 12 h. After completion, the reaction mixture was concentrated in vacuo, diluted with EtOAc and filtered. The organic layer was washed with water, saturated NaCl solution, dried over Na₂SO₄ and concentrated in vacuo to afford the title intermediate 58 as a yellow solid (1.3 g, yield 54.16%).

tert-Butyl 3-(2-chloro-4-cyano-5-methylphenyl)propanoate (59). To a stirred solution of intermediate 58 (1.3 g, 4.6 mmol) in MeOH (4 mL) was added 5% Pd/C (100 mg) and the reaction was stirred under hydrogen atmosphere for 16 h. After completion the reaction mixture was filtered and the filtrate was concentrated in vacuo to afford the title intermediate 59 as a yellow semi-solid (1.15 g, yield 88.5%).

tert-Butyl 3-(2-chloro-4-(N′-hydroxycarbamimidoyl)-5-methylphenyl)propanoate (60): To a stirred solution of hydroxylamine hydrochloride (1.71 g, 24.7 mmol) in EtOH (5 mL) was added triethylamine (3.4 mL, 25 mmol) and the mixture stirred for 30 min. Intermediate 59 (1.150 g, 4.120 mmol) in EtOH (25 mL) was added and the reaction was stirred at 80° C. for 3 h. After completion, the reaction mixture was concentrated in vacuo and the residue was dissolved in EtOAc. The organic layer was washed with water, dried over Na₂SO₄ and concentrated in vacuo to afford the title intermediate 60 as a yellow semi-solid (1.0 g, yield 78%).

tert-Butyl 3-(4-(5-(8-bromo-6-(trifluoromethyl)imidazo[1,2-a]pyridin-2-yl)-1,2,4-oxadiazol-3-yl)-2-chloro-5-methylphenyl)propanoate (61). To a stirred solution of hydroxyimidate 60 (0.730 g, 2.33 mmol) in DMF (10 mL) was added acid 37 (0.864 g, 2.79 mmol) followed by addition of EDCI.HCl (0.671 g, 3.50 mmol) and HOBT (0.472 g, 3.50 mmol). The resulting mixture was stirred at 100° C. for 15 h. After completion, the reaction mixture was concentrated in vacuo and the crude compound was purified by column chromatography (15% EtOAc/hexane as eluent) to afford the title intermediate 61 as a yellow oil (0.510 g, yield 37.3%).

3-(4-(5-(8-bromo-6-(trifluoromethyl)imidazo[1,2-a]pyridin-2-yl)-1,2,4-oxadiazol-3-yl)-2-chloro-5-methylphenyl)propanoic acid. A solution of intermediate 61 (0.530 g, 0.90 mmol) in 30% TFA/DCM (10 mL) was stirred at room temperature for 30 min. After completion, the reaction mixture was concentrated in vacuo and the resulting solid was washed successively with diethyl ether and iPrOH and dried completely to afford the title compound as a white solid (296 mg, yield 61.8%). ¹H NMR (400 MHz, DMSO-d₆) δ 9.39 (s, 1H), 9.14 (s, 1H), 8.19 (s, 1H), 8.12 (s, 1H), 7.42 (s, 1H), 2.89 (t, 3H), 2.62 (t, 2H), 2.61 (s, 3H); MS (EI) for C₂₀H₁₃BrClF₃N₄O₃, found 531 (MH+).

### Example 12

### 2-Amino-3-(3-chloro-4-(5-(8-chloro-6-(trifluoromethyl)imidazo[1,2-a]pyridin-2-yl)-1,2,4-oxadiazol-3-yl)phenoxy)propanoic acid hydrochloride

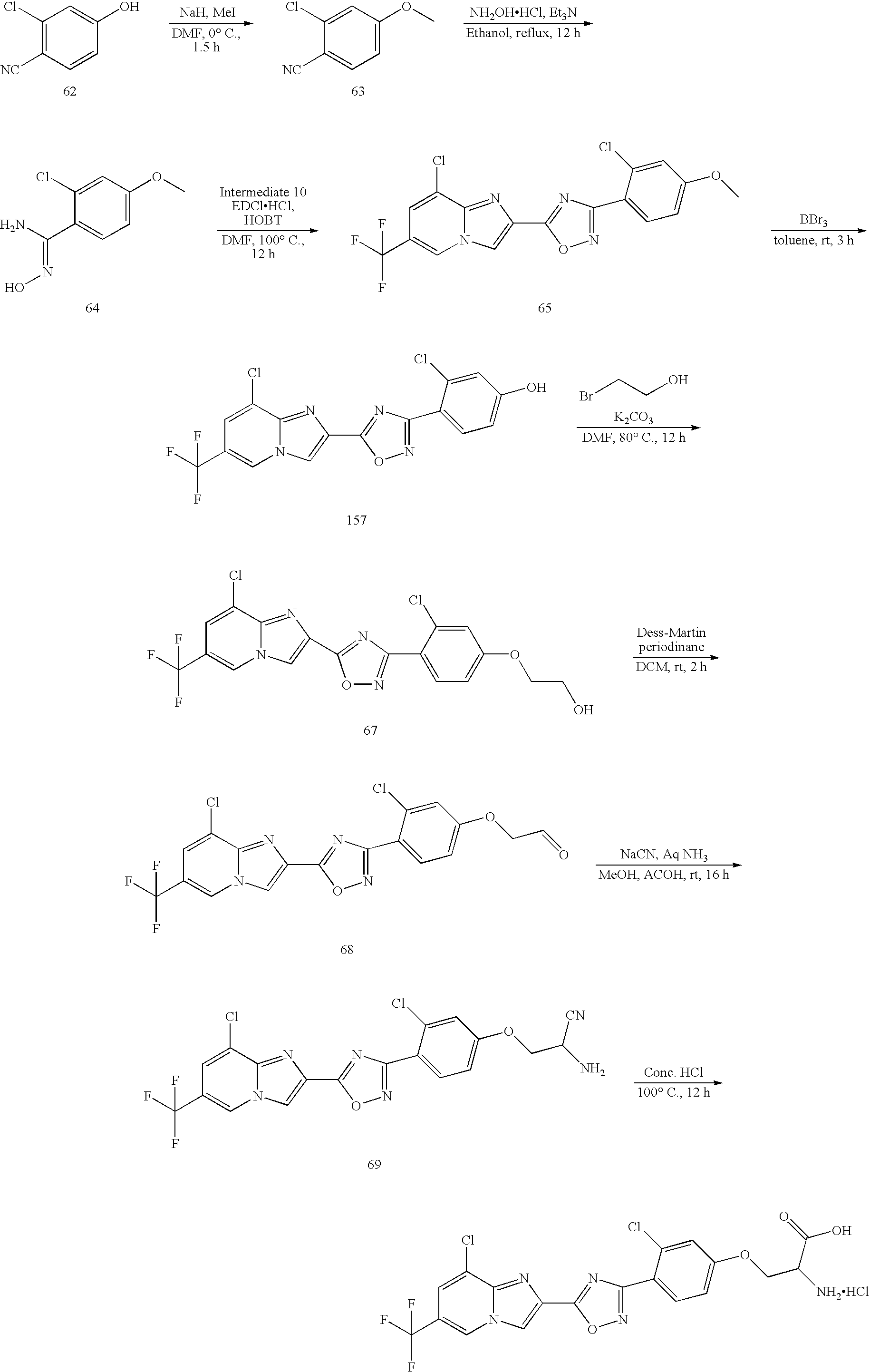

2-Chloro-4-methoxy benzonitrile (63). To a stirred solution of 2-chloro-4-hydroxy benzonitrile 62 (2.5 g, 16 mmol) in DMF (25 mL) was added NaH (0.431 g, 18.0 mmol) at 0° C. After 30 min, methyl iodide (3.47 g, 24.4 mmol) was added dropwise and the reaction mixture was stirred at room temperature for 1 h. After completion, the reaction mixture was quenched with ice water and extracted with diethyl ether. The organic layer was washed with water, saturated NaCl solution, dried over Na₂SO₄ and concentrated in vacuo to afford the title intermediate 63 as a brown solid (2.7 g, yield 98%).

2-Chloro-4-methoxy-N′-hydroxy-benzimidamide (64). To a stirred solution of compound 63 (2.5 g, 15 mmol) in EtOH (25 mL) was added hydroxylamine hydrochloride (6.19 g, 89.7 mmol) and triethylamine (9.06 g, 89.7 mmol). The reaction was then heated to reflux for 12 h. After completion, the reaction mixture was concentrated and the residue was diluted with water and extracted with EtOAc. The combined organic layers were washed with water, saturated NaCl solution, dried over Na₂SO₄ and concentrated in vacuo to afford the title intermediate 64 as a green solid (2.5 g, yield 84%).

3-(2-Chloro-4-methoxyphenyl)-5-(8-chloro-6-(trifluoromethyl)imidazo[1,2-a]pyridin-2-yl)-1,2,4-oxadiazole (65). To a stirred solution of hydroxyimidate 64 (0.90 g, 4.5 mmol) in DMF (15 mL) was added Intermediate 10 (1 g, 3.78 mmol), EDCI.HCl (1.08 g, 5.60 mmol) and HOBT (0.76 g, 5.6 mmol). The reaction was stirred at room temperature for 1 h and then at 100° C. for 12 h. The reaction mixture was quenched with water and extracted with EtOAc. The combined organic layer was washed with 5% K₂CO₃, 1N HCl solution, water, saturated NaCl solution, dried over Na₂SO₄ and concentrated in vacuo to afford the title intermediate 65 as a white solid (0.8 g, yield 50%).

3-Chloro-4-(5-(8-chloro-6-(trifluoromethyl)imidazo[1,2-a]pyridin-2-yl)-1,2,4-oxadiazol-3-yl)phenol. To a stirred solution of intermediate 65 (0.800 g, 1.86 mmol) in toluene (10 mL) was added boron tribromide (2.66 g 10.6 mmol) at 15° C. and the reaction mixture was stirred at room temperature for 3 h. After completion, the reaction mixture was quenched with ice water and extracted with EtOAc. The organic layer washed with saturated ammonium chloride solution, saturated NaCl solution, dried over Na₂SO₄ and concentrated in vacuo to afford the title intermediate 157 as a brown solid (0.70 g, yield 90%). MS (EI) for C₁₆H₇Cl₂F₃N₄O₂, found 414.9 (MH+).

2-(3-Chloro-4-(5-(8-chloro-6-(trifluoromethyl)imidazo[1,2-a]pyridin-2-yl)-1,2,4-oxadiazol-3-yl)phenoxy)ethanol (67). To a stirred solution of intermediate 157 (1.0 g, 2.4 mmol) in DMF was added K₂CO₃ (1.66 g, 12.0 mmol) and 2-bromoethanol (1.5 g, 12 mmol) and the reaction mixture was stirred at 80° C. for 12 h. After completion, the reaction mixture was quenched with ice water and extracted with EtOAc. The organic layer was washed with water, saturated NaCl solution, dried over Na₂SO₄ and concentrated in vacuo to afford the title intermediate 67 as a light brown solid (1 g, yield 90%).

2-(3-Chloro-4-(5-(8-chloro-6-(trifluoromethyl)imidazo[1,2-a]pyridin-2-yl)-1,2,4-oxadiazol-3-yl)phenoxy)acetaldehyde (68). To a stirred solution of intermediate 67 (2 g, 4.3 mmol) in DCM (10 mL) was added Dess-Martin periodinane reagent (2.03 g, 4.7 mmol) and the reaction mixture was stirred at room temperature for 2 h. After completion, the reaction mixture was quenched with saturated sodium bicarbonate (10 mL) and Na₂S₂O₃ (10 mL) and extracted with DCM. The organic layer was washed with water, saturated NaCl solution, dried over Na₂SO₄ and concentrated in vacuo to afford the title intermediate 68 as a brown solid (1.7 g, yield 85%).

2-Amino-3-(3-chloro-4-(5-(8-chloro-6-(trifluoromethyl)imidazo[1,2-a]pyridin-2-yl)-1,2,4-oxadiazol-3-yl)phenoxy)propanenitrile (69). To a stirred solution of intermediate 68 (0.70 g, 1.5 mmol) in MeOH (10 mL) was added aq. NH₃ (0.1565 g, 4.47 mmol) and the pH adjusted to 4-5 with acetic acid. After stirring at room temperature for 1 h, NaCN (0.1509 g, 3.07 mmol) was added and stirring continued for 1 h. Aq. NH₃ (21 mL) was added and the resulting reaction mixture was stirred for 16 h at room temperature. After completion, the reaction mixture was concentrated in vacuo, diluted with water and extracted with EtOAc. The combined organic layers were washed with water, saturated NaCl solution, dried over Na₂SO₄ and concentrated in vacuo to afford the title intermediate 69 as a brown solid (0.5 g, yield 68%).

2-Amino-3-(3-chloro-4-(5-(8-chloro-6-(trifluoromethyl)imidazo[1,2-a]pyridin-2-yl)-1,2,4-oxadiazol-3-yl)phenoxy)propanoic acid hydrochloride. A solution of intermediate 69 (0.50 g, 1.0 mmol) in concentrated HCl (5 mL) was stirred at 100° C. for 2 h. After completion, the reaction mixture was concentrated in vacuo and purified by preparative HPLC to afford the title compound as a white solid (35 mg, 7.0% yield). ¹H-NMR (400 MHz, DMSO-d₆) δ 9.35 (s, 1H), 9.1 (s, 1H), 8.2 (m, 2H), 7.35 (s, 1H), 7.2 (d, 1H), 4.5 (m, 1H), 3.3 (m, 2H); MS (EI) for C₁₉H₁₂Cl₂F₃N₅O₄, found 501.84 (MH+).

The following compounds were prepared using the same or analogous synthetic techniques in Example 12 and substituting with appropriate reagents, which were commercially available or were prepared using procedures herein or procedures known to one of ordinary skill in the art.

2-Amino-3-[(3-chloro-4-{5-[8-chloro-6-(trifluoromethyl)imidazo[1,2-a]pyridin-2-yl]-1,2,4-oxadiazol-3-yl}phenyl)oxy]propan-1-ol. MS (EI) for C₁₉H₁₄Cl₂F₃N₅O₃, found 488.0 (MH+).

2-Amino-3-[(2,5-dichloro-4-{5-[8-chloro-6-(trifluoromethyl)imidazo[1,2-a]pyridin-2-yl]-1,2,4-oxadiazol-3-yl}phenyl)oxy]propan-1-ol. MS (EI) for C₁₉H₁₃Cl₃F₃N₅O₃, found 522.0 (MH+).

### Example 13

### 2,5-Dichloro-4-(5-(8-chloro-6-(trifluoromethyl)imidazo(1,2-a)pyridine-2-yl)-1,2,4-oxadiazole-3-yl)phenol

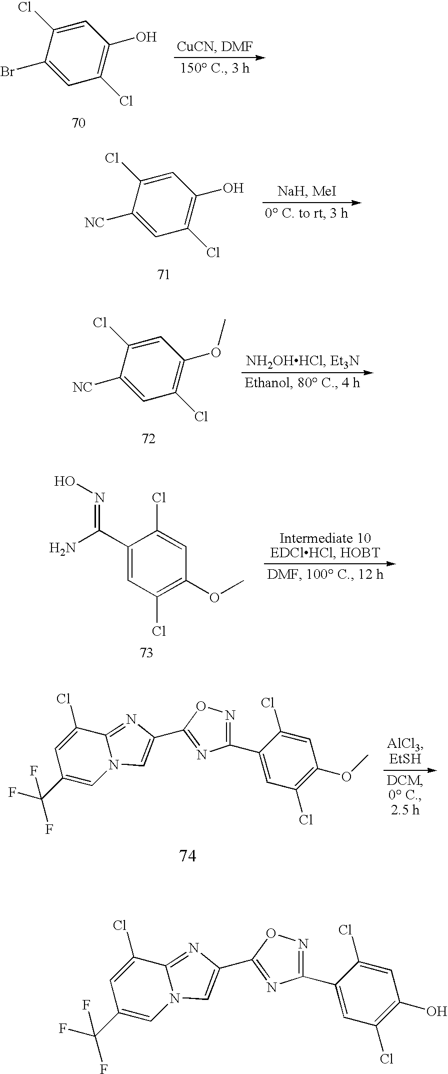

2,5-Dichloro-4-hydroxy benzonitrile (71). To a stirred solution of 2,5-dichloro-4-bromophenol 70 (10 g, 41 mmol) in DMF (50 mL) was added cuprous cyanide (4.83 g, 53.8 mmol) and the reaction was stirred at 150° C. for 3 h. After completion, the reaction mixture was concentrated in vacuo. To the residue, water and EtOAc were added and the biphasic mixture filtered through celite. The filtrate was extracted with EtOAc and the combined organic layers were dried over Na₂SO₄ and concentrated in vacuo. The crude compound was purified by column chromatography, using 15% EtOAc/hexane as eluent, to afford the title intermediate 71 as a white solid (7.7 g, yield 100%).

2,5-Dichloro-4-methoxy benzonitrile (72). To a stirred solution of 2,5-dichloro-4-cyanophenol 71 (11.0 g, 58.5 mmol) in DMF (40 mL) was added NaH (4.3 g, 109 mmol) in small portions at 0° C. and stirred for 30 min at 0° C. Methyl iodide (11 mL, 176 mmol) was added dropwise and after addition the reaction was allowed to warm to room temperature and was stirred for 3 h. After completion, the reaction mixture was cooled to 0° C. and quenched with ice water. The precipitated solid was collected by filtration, washed well with water and dried completely to afford the title intermediate 72 as a white solid (6.4 g, yield 54%).

2,5-Dichloro-N-hydroxy-4-methoxy benzimidine (73): To a stirred solution of hydroxylamine hydrochloride (8.8 g, 127 mmol) in EtOH (50 mL) was added triethylamine (16.1 g, 158 mmol) and the reaction was stirred for 30 min. To this mixture, 4-methoxy-2,5-dichlorobenzonitrile 72 (6.4 g, 32 mmol) was added and the reaction mixture was stirred at 80° C. for 4 h. After completion, the reaction mixture was concentrated in vacuo and the residue was dissolved in EtOAc. The organic phase was washed with water, dried over Na₂SO₄ and concentrated under high vacuum to afford the title intermediate 73 as a white solid (2.1 g, yield 21%).

5-(8-Chloro-6-(trifluoromethyl)imidazol(1,2-a)pyridine-2-yl-3-(2,5-dichloro-4-methoxyphenyl)-1,2,4-oxadizole (74). To a stirred solution of Intermediate 10 (2.86 g, 10.8 mmol) in DMF (5 mL) was added EDCI.HCl (2.07 g, 10.8 mmol) followed by HOBT (1.46 g, 10.8 mmol). After stirring for 15 min, hydroxyimidate 73 (2.12 g, 9.02 mmol) was added and the reaction mixture was stirred at 100° C. for 12 h. After completion, the reaction mixture was concentrated in vacuo and the residue was purified by crystallization using iPrOH to afford the title intermediate 74 as a white solid (2.89 g, yield 69.1%).

2,5-Dichloro-4-(5-(8-chloro-6-(trifluoromethyl)imidazo(1,2-a)pyridine-2-yl)-1,2,4-oxadiazole-3-yl)phenol. To a cold solution of 74 (2.89 g, 6.23 mmol) in DCM (20 mL) was added AlCl₃ (4.15 g, 31.16 mmol) in small portions under argon such that the reaction temperature was maintained below 10° C. The light brown suspension was stirred for 10 min and then EtSH (2.30 mL, 31.16 mmol) was added dropwise at such a rate that the reaction temperature was maintained below 5° C. After 2.5 h of stirring below 10° C., the reaction mixture was slowly poured into ice water with strong agitation. The organic layer was separated and the aqueous layer was extracted with DCM. The combined DCM layers were washed with water and dried over Na₂SO₄. The solvent was removed under reduced pressure, giving a solid. The solid was azeotropically distilled with toluene to afford the title compound as a light yellow solid (2.4 g, yield 86%). ¹H NMR (400 MHz, DMSO-d₆) δ 11.58 (s, 1H), 9.28 (s, 1H), 9.12 (s, 1H), 8.10 (s, 2H), 7.20 (s, 1H); MS (EI) for C₁₆H₆Cl₃F₃N₄O₂, found 449 (MH+).

Using the same or analogous synthetic techniques in Example 13 and substituting with appropriate reagents (prepared using procedures described herein), the following compound was prepared. 4-{5-[8-Bromo-6-(trifluoromethyl)imidazo[1,2-a]pyridin-2-yl]-1,2,4-oxadiazol-3-yl}-2,5-dichlorophenol. MS (EI) for C₁₆H₆BrCl₂F₃N₄O₂, found 495 (MH+).

### Example 14

### 3-(2,5-Dichloro-4-(5-(8-chloro-6-(trifluoromethyl)imidazo[1,2-a]pyridin-2-yl)-1,2,4-oxadiazol-3-yl)phenoxy)propane-1,2-diol

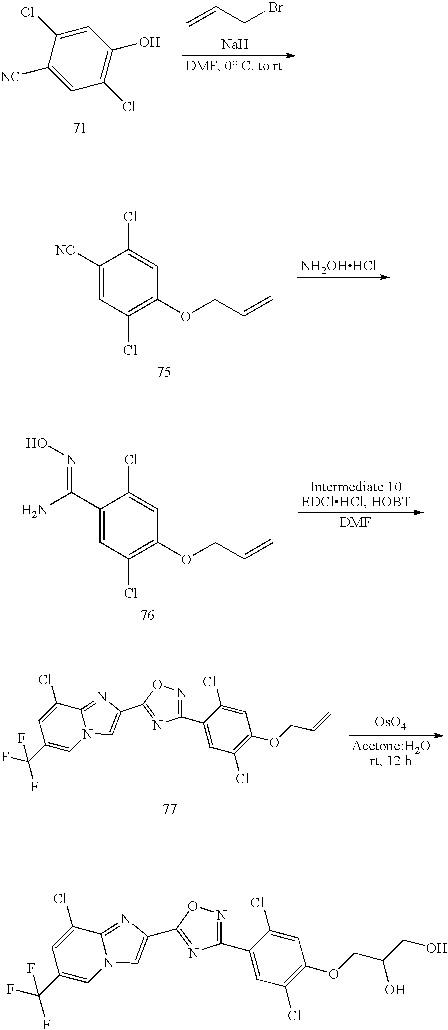

4-(Allyloxy)-2,5-dichlorobenzonitrile (75). To intermediate 71 (1.72 g, 9.15 mmol) in dry DMF (20 mL), was added NaH (475 mg, 0.011 mole, 60% dispersion in oil) at 0° C. and stirred for 20 min. To the resulting reaction mixture, allyl bromide (1.5 ml, 0.018 mol) was added dropwise at 0° C. and the mixture stirred for an additional 3 h at room temperature. Excess NaH was quenched by ice, and the resulting mixture extracted with EtOAc. The combined extracts were washed with water, saturated NaCl, dried over Na₂SO₄, and concentrated. The resulting residue was purified by column chromatography using EtOAc:hexane as eluent to give intermediate 75 (1.22 g, 59.0%) as a white solid.

4-(Allyloxy)-2,5-dichloro-N′-hydroxybenzimidamide (76). To an ethanolic solution (10 mL) of hydroxylamine hydrochloride (1.496 g, 0.022 mol), triethylamine (3.7 mL, 0.027 mol) was added slowly. The resulting mixture was stirred at room temperature for 1 h followed by the addition of cyano intermediate 75 (1.22 g, 5.35 mmol) in EtOH. The resulting mixture was stirred at room temperature for 0.5 h, then heated to 80° C. overnight. The reaction mixture was concentrated in vacuo to remove EtOH, then extracted with EtOAc. The combined organic phases were washed with water, saturated NaCl, dried over Na₂SO₄, and concentrated to give intermediate 76 (1.17 g, 84%), which was used in subsequent reaction without further purification.

3-(4-(Allyloxy)-2,5-dichlorophenyl)-5-(8-chloro-6-(trifluoromethyl)imidazo[1,2-a]pyridin-2-yl)-1,2,4-oxadiazole (77). To a solution of Intermediate 10 (1.54 g, 5.82 mol) in dry DMF (10 mL) was added EDCI.HCl (1.29 g, 6.73 mmol) and HOBT (0.912 g, 6.76 mol) and the mixture stirred at room temperature for 1 h. Hydroxyimidate 76 (1.170 g, 4.481 mmol) was added in dry DMF (5 mL) and the mixture stirred at room temperature for 0.5 h followed by heating to 100° C. for 14 h. The reaction mixture was concentrated in vacuo and the resulting residue was partitioned between in EtOAc and water. The aqueous phase was further extracted with EtOAc. The organic layer was washed with saturated NaCl, dried over Na₂SO₄ and concentrated. The crude product was purified by column chromatography using EtOAc/hexane to afford intermediate 77 (750 mg, 34.7%) as a white solid.

3-(2,5-Dichloro-4-(5-(8-chloro-6-(trifluoromethyl)imidazo[1,2-a]pyridin-2-yl)-1,2,4-oxadiazol-3-yl)phenoxy)propane-1,2-diol. The allyl intermediate 77 (750 mg, 1.5 mmol) was dissolved in acetone:water (9:1, 5 mL) to which was added OsO₄ (0.2 mL, 0.1 M solution in toluene) and NMO (2 mL). The resulting reaction mixture was stirred overnight at room temperature. After completion, the reaction mixture was quenched with saturated sodium sulfite solution and stirred for an additional 45 min. The resulting solid was filtered, washed with water followed by ether to give the title compound (561 mg, 70%) as a white solid. ¹H-NMR (400 MHz, DMSO-d₆) δ 9.20 (s, 1H), 8.90 (s, 1H), 8.18 (s, 1H), 7.80 (s, 1H), 7.40 (s, 1H), 4.38-4.00 (4H, m), 3.70-3.80 (m, 3H); MS (EI) for C₁₉H₁₂Cl₃F₃N₄O₄, found 523 (MH+).

The following compounds were prepared using the same or analogous synthetic techniques in Example 14 and substituting with appropriate reagents, which were commercially available or were prepared using procedures herein or procedures known to one of ordinary skill in the art.

3-[(2,6-Dichloro-4-{5-[8-chloro-6-(trifluoromethyl)imidazo[1,2-a]pyridin-2-yl]-1,2,4-oxadiazol-3-yl}phenyl)oxy]propane-1,2-diol. ¹H-NMR (400 MHz, DMSO-d₆) δ 9.35 (s, 1H), 9.07 (s, 1H), 8.14 (s, 1H), 8.07 (d, 1H), 5.04 (d, 1H), 4.71 (t, 1H), 4.09 (m, 2H), 3.91 (m, 1H), 3.50 (m, 2H); MS (EI) for C₁₉H₁₂Cl₃F₃N₄O₄, found 523.0 (MH+).

3-({2,5-Dichloro-4-[5-(8-chloro-6-iodoimidazo[1,2-a]pyridin-2-yl)-1,2,4-oxadiazol-3-yl]phenyl}oxy)propane-1,2-diol. MS (EI) for C₁₈H₁₂Cl₃1N₄O₄, found 581 (MH+).

3-[(5-Chloro-4-{5-[8-chloro-6-(trifluoromethyl)imidazo[1,2-a]pyridin-2-yl]-1,2,4-oxadiazol-3-yl}-2-fluorophenyl)oxy]propane-1,2-diol. MS (EI) for C₁₉H₁₂Cl₂F₄N₄O₄, found 507.0 (MH+).

3-[(4-{5-[8-bromo-6-(trifluoromethyl)imidazo[1,2-a]pyridin-2-yl]-1,2,4-oxadiazol-3-yl}-2,5-dichlorophenyl)oxy]propane-1,2-diol. MS (EI) for C₁₉H₁₂BrCl₂F₃N₄O₄, found 567 (MH+).

### Example 15

### 1-Amino-3-(2,5-dichloro-4-(5-(8-chloro-6-(trifluoromethyl)imidazo(1,2-a)pyridine-2-yl)1,2,4-oxadiazol-3-yl)phenoxy)propan-2-ol

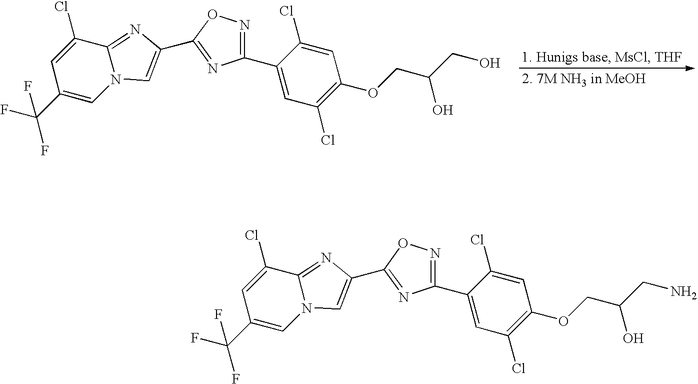

1-Amino-3-(2,5-dichloro-4-(5-(8-chloro-6(trifluoromethyl)imidazo(1,2)pyridine-2-yl)1,2,4-oxadiazol-3-yl)phenoxy)propan-2-ol. To a stirred solution of intermediate 3-(2,5-dichloro-4-(5-(8-chloro-6-(trifluoromethyl)imidazo[1,2-a]pyridin-2-yl)-1,2,4-oxadiazol-3-yl)phenoxy)propane-1,2-diol (270 mg, 0.52 mmol), prepared as described in Example 14, in THF (5 mL) was added Hunig's base (0.2 mL) followed by methanesulfonylchloride (0.06 g) at 0° C. After addition, the reaction mixture was stirred at room temperature for 16 h. After completion, the reaction mixture was concentrated in vacuo. To the residue, 7 M NH₃ in MeOH (5 mL) was added and the resulting mixture heated in a sealed tube at 60° C. for 12 h. The reaction mixture was cooled to room temperature and concentrated in vacuo. The crude product was purified by preparative HPLC to afford the title compound as a white solid (80 mg, yield 29%). ¹H NMR (400 MHz, DMSO-d₆) δ 9.28 (s, 1H), 9.20 (s, 1H), 8.18 (s, 1H), 8.14 (s, 1H), 7.81 (br s, 2H, —NH₂), 7.59 (s, 1H), 5.95 (br s, 1H), 4.23 (m, 2H), 4.15 (br s, 1H), 2.85-3.20 (m, 2H); MS (EI) for C₁₉H₁₃Cl₃F₃N₅O₃, found 524 (MH+).

The following compounds were prepared using the same or analogous synthetic techniques in Example 15 and substituting with appropriate reagents, which were commercially available or were prepared using procedures herein or procedures known to one of ordinary skill in the art.

1-Amino-3-[(5-chloro-4-{5-[8-chloro-6-(trifluoromethyl)imidazo[1,2-a]pyridin-2-yl]-1,2,4-oxadiazol-3-yl}-2-fluorophenyl)oxy]propan-2-ol. MS (EI) for C₁₉H₁₃Cl₂F₄N₅O₃, found 506.0 (MH+).

1-Amino-3-[(3-chloro-4-{5-[8-chloro-6-(trifluoromethyl)imidazo[1,2-a]pyridin-2-yl]-1,2,4-oxadiazol-3-yl}phenyl)oxy]propan-2-ol. MS (EI) for C₁₉H₁₄Cl₂F₃N₅O₃, found 488.0 (MH+).

### Example 16

### 3-(3-Chloro-4-(5-(8-chloro-6-(trifluoromethyl)imidazo[1,2-a]pyridin-2-yl)-1,2,4-oxadiazol-3-yl)phenoxy)propane-1,2-diol

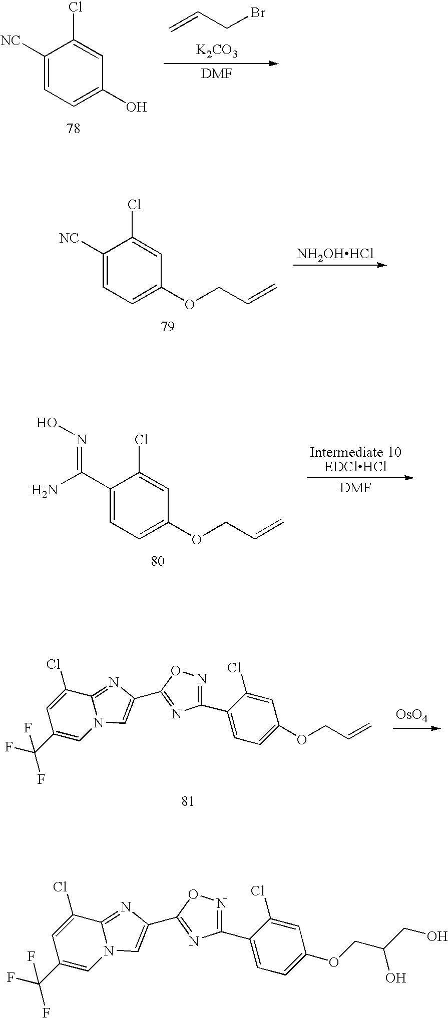

4-(Allyloxy)-2-chlorobenzonitrile (79). To intermediate 78 (3 g, 0.02 mol) in dry DMF (25 mL), was added NaH (1.0 g, 0.025 mol, 60% dispersion in oil) at 0° C. and stirred for 20 min. To the reaction mixture, allyl bromide (3.37 mL, 0.039 mol) was added dropwise at 0° C. and stirred for additional 3 h at room temperature. Excess NaH was quenched with ice, and the resulting reaction mixture extracted with EtOAc. The combined extracts were washed with water, saturated NaCl, dried over Na₂SO₄, and concentrated. The resulting residue was purified by column chromatography, using EtOAc:hexane as eluent, to give intermediate 79 (3 g, 79%) as a colorless solid.

4-(Allyloxy)-2-chloro-N′-hydroxybenzimidamide (80). To an ethanolic solution (30 mL) of hydroxylamine hydrochloride (6.8 g, 0.98 mol), triethylamine (13.7 mL, 0.098 mol) was added slowly and the mixture stirred at room temperature for 1 h. Cyano intermediate 79 (3.2 g, 0.016 mol) in EtOH was added and the resulting mixture stirred at room temperature for 0.5 h followed by heating to 80° C. for 3 h. The reaction mixture was concentrated in vacuo to remove excess of EtOH and extracted with EtOAc. The combined organic fractions were washed with water, saturated NaCl, dried over Na₂SO₄ and concentrated to give intermediate 80 (3.9 g), which was used in subsequent reactions without further purification.

3-(4-(Allyloxy)-2-chlorophenyl)-5-(8-chloro-6-(trifluoromethyl)imidazo[1,2-a]pyridin-2-yl)-1,2,4-oxadiazole (81). To a solution of Intermediate 10 (6.8 g, 0.025 mol) in dry acetonitrile (35 mL), was added EDCI.HCl (4.94 g, 0.025 mol) and the mixture stirred for 30 min at room temperature. Hydroxyimidate 80 (3.9 g, 0.017 mol) was added in dry acetonitrile and the mixture stirred at room temperature for 0.5 h followed by heating to 100° C. for 12 h. The reaction mixture was concentrated in vacuo and the residue partitioned between EtOAc and water. The aqueous phase was separated and further extracted with EtOAc. The organic layers were washed with saturated NaCl, dried over Na₂SO₄ and concentrated. The crude product was purified by crystallization from iPrOH to afford intermediate 81 (5.2 g, 67%) as a white solid.

3-(3-Chloro-4-(548-chloro-6-(trifluoromethyl)imidazo[1,2-a]pyridin-2-yl)-1,2,4-oxadiazol-3-yl)phenoxy)propane-1,2-diol. The allyl compound 81 (1 g, 0.002 mol) was dissolved in acetone:water (9:1, 10 mL), followed by the addition of OsO₄ (0.3 mL, 0.1 M solution in toluene) and NMO (4 mL). The reaction mixture was stirred overnight at room temperature. After completion, the reaction mixture was quenched with saturated sodium sulfite solution. The stirring was continued for an additional 45 min and the resulting solid was filtered and washed with water and ether to give the title compound (752 mg, 70%) as a white solid. ¹H NMR (400 MHz, DMSO-d₆) δ 9.39 (s, 1H), 8.87 (s, 1H), 8.22 (d, 1H), 8.00 (s, 1H), 7.29 (s, 1H), 7.20 (d, 1H), 5.0 (d, 1H), 4.78 (t, 1H), 4.22-4.00(m, 3H), 3.90 (m, 2H); MS (EI) for C₁₉H₁₃Cl₂F₃N₄O₄, found 489 (MH+).

### Example 17

### 2-(2,5-Dichloro-4-(3-(8-chloro-6-(trifluoromethyl)imidazo[1,2-a]pyridin-2-yl)-1,2,4-oxadiazol-5-yl)phenoxy)propan-1-ol

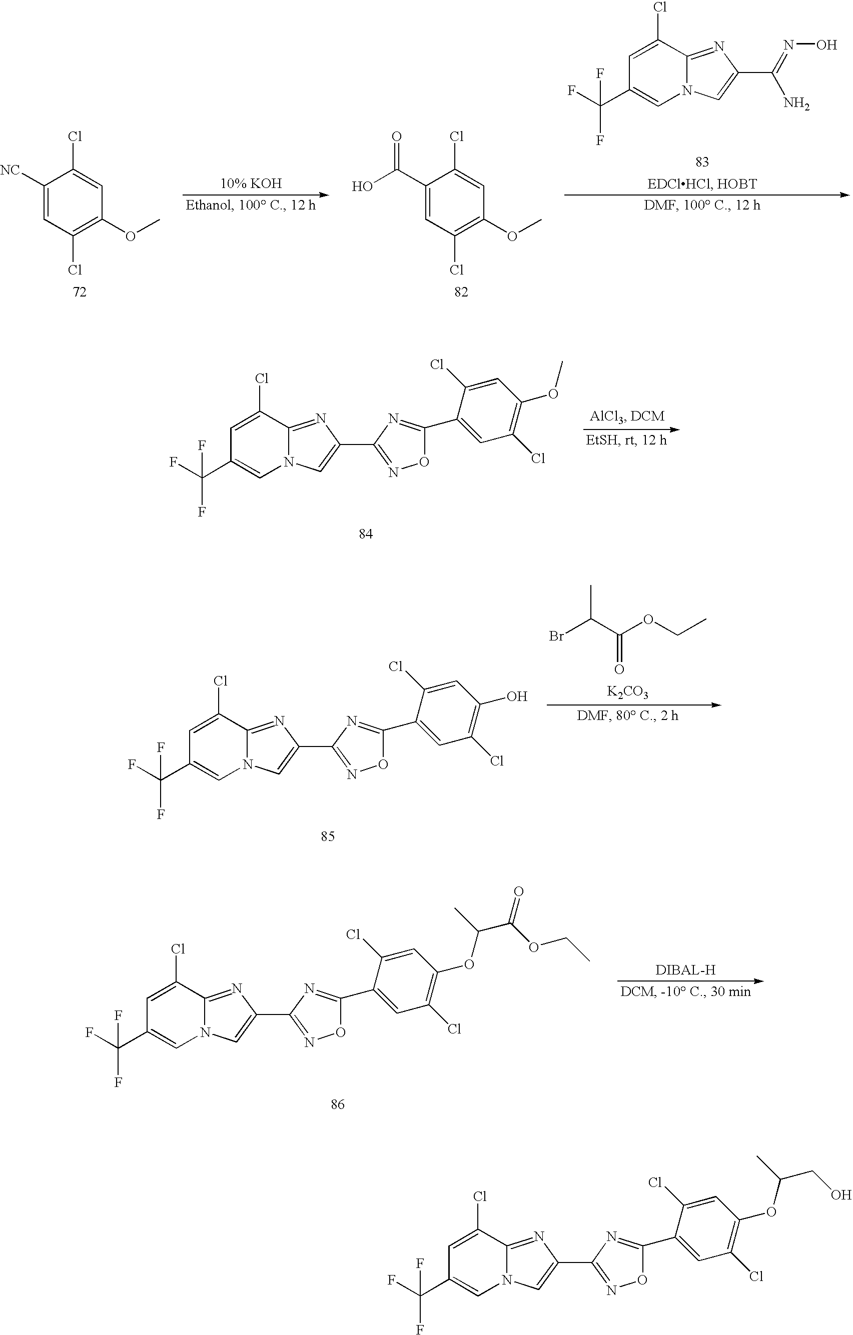

2,5-Dichloro-4-hydroxy benzoic acid (82). To a stirred solution of intermediate 72 (3.4 g, 17 mmol) in EtOH (5 mL) was added 10% aqueous KOH solution (30 mL) and the resulting mixture was stirred at 100° C. for 12 h. After completion, solvent was removed in vacuo and the aqueous phase was neutralized with 2 N HCl and extracted with EtOAc. The organic layer was washed with water, saturated NaCl solution, dried over Na₂SO₄ and concentrated to afford the title intermediate 82 as a white solid (1.65 g, yield 44%).

3-(8-Chloro-6-(trifluoromethyl)imidazo[1,2-a]pyridin-2-yl)-5-(2,5-dichloro-4-methoxyphenyl)-1,2,4-oxadiazole (84). To a stirred solution of Intermediate 83 (1.6 g, 7.2 mmol), prepared as described above, in DMF (5 mL) was added acid 82 (2.0 g, 7.2 mmol) followed by addition of EDCI.HCl (1.4 g, 7.2 mmol) and HOBT (0.97 g, 7.2 mmol). The resulting mixture was stirred at room temperature for 1 h, followed by 100° C. for 12 h. After completion, the reaction mixture was concentrated in vacuo and the crude compound was purified by column chromatography (10% EtOAc/hexane as eluent) to afford the title intermediate 84 as a white solid (1.5 g, yield 45%).

5-Chloro-4-(5-(8-chloro-6-(trifluoromethyl)imidazo[1,2-a]pyridin-2-yl)-1,2,4-oxadiazol-3-yl)-2-fluorophenol (85). To a stirred solution of intermediate 84 (1.4 g, 3.0 mmol) in DCM (15 mL) cooled to 0° C. was added AlCl₃ 15.0 mmol) slowly over a period of 20 min. After stirring the reaction at 0° C. for 30 min, EtSH (12 mL, 15 mmol) was added dropwise at 0° C. After addition was complete, the reaction mixture was allowed to warm to room temperature and stirred for two h. After completion, the reaction mixture was quenched with ice water and extracted with EtOAc. The combined organic layers were washed with water, saturated NaCl solution, dried over Na₂SO₄ and concentrated. The crude product was purified by column chromatography (12% EtOAc/hexane as eluent) to afford the title intermediate 85 as an off-white solid (1.2 g, yield 89%).

2-(5-Chloro-4-(5-(8-chloro-6-(trifluoromethyl)imidazo[1,2-a]pyridin-2-yl)-1,2,4-oxadiazol-3-yl)-2-fluorophenoxy)ethanol (86). To a stirred solution of 85 (1.2 g, 2.7 mmol) in DMF (10 mL) was added K₂CO₃ (1.5 g, 11 mmol) followed by ethyl 2-bromopropionate (1.4 mL, 11 mmol) and the reaction was stirred at 80° C. for 2 h. After completion, the reaction mixture was concentrated in vacuo, diluted with water and extracted with EtOAc. The combined organic layers were washed with water, saturated NaCl solution, dried over Na₂SO₄ and concentrated to afford the title intermediate 86 as a white solid (1.1 g, yield 75%).

2-(2,5-Dichloro-4-(3-(8-chloro-6-(trifluoromethyl)imidazo[1,2-a]pyridin-2-yl)-1,2,4-oxadiazol-5-yl)phenoxy)propan-1-ol. To a stirred solution of ester 86 (1.0 g, 1.8 mmol) in DCM cooled to −10° C. was added DIBAL (1 M solution in THF, 14 mL, 5.5 mmol) dropwise over a period of 15 min. After addition was complete, the reaction mixture was stirred at −10° C. for 30 min. After completion, the reaction mixture was slowly quenched with saturated ammonium chloride solution at −10° C. The reaction mixture was extracted with EtOAc and the combined organic layers were dried over Na₂SO₄ and concentrated in vacuo to afford the title compound as a white solid (0.680 g, yield 73.9%). ¹H NMR (400 MHz, DMSO-d₆) δ 9.29 (s, 1H), 9.88 (s, 1H), 8.22 (s, 1H), 8.0 (s, 1H), 7.64 (s, 1H), 5.10 (m, 1H), 4.82 (m, 1H), 3.60 (m, 2H), 1.25 (d, 3H); MS (EI) for C₁₉H₁₂Cl₃F₃N₄O₃, found 507 (MH+).

The following compounds were prepared using the same or analogous synthetic techniques in Example 17 and substituting with appropriate reagents, which were commercially available or were prepared using procedures herein or procedures known to one of ordinary skill in the art.

2-[(3-Chloro-4-{3-[8-chloro-6-(trifluoromethyl)imidazo[1,2-a]pyridin-2-yl]-1,2,4-oxadiazol-5-yl}phenyl)oxy]ethanamine. MS (EI) for C₁₈H₁₂Cl₂F₃N₅O₂, found 458.0 (MH+).

2-[(5-Chloro-4-{3-[8-chloro-6-(trifluoromethyl)imidazo[1,2-a]pyridin-2-yl]-1,2,4-oxadiazol-5-yl}-2-fluorophenyl)oxy]propan-1-ol. ¹H NMR (400 MHz, DMSO-d₆) δ 9.31 (s, 1H), 8.83 (s, 1H), 8.06 (d, 1H), 7.98 (s, 1H), 7.67 (d, 1H), 5.02 (t, 1H), 4.79 (m, 1H), 3.59 (m, 2H), 1.28 (d, 3H); MS (EI) for C₁₉H₁₂Cl₂F₄N₄O₃, found 491 (MH+).

2-[(5-Chloro-4-{3-[8-chloro-6-(trifluoromethyl)imidazo[1,2-a]pyridin-2-yl]-1,2,4-oxadiazol-5-yl}-2-fluorophenyl)oxy]propanoic acid. MS (EI) for C₁₉H₁₀Cl₂F₄N₄O₄, found 505 (MH+).

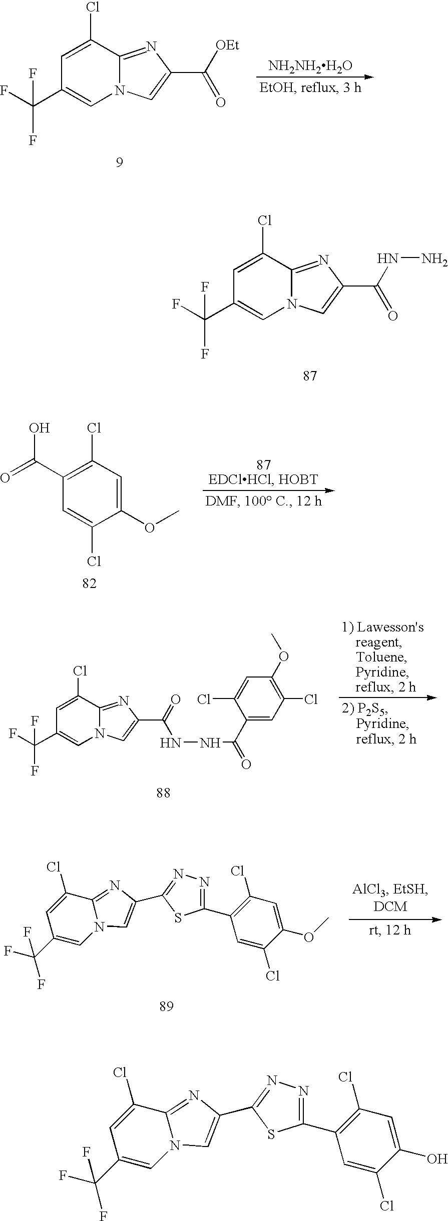

### Example 18

8-Chloro-6-(trifluoromethyl)imidazo[1,2-a]pyridine-2-carbohydrazide (87). To a stirred solution of ester 9 (15 g, 51 mmol), prepared as described in the synthesis of Intermediate 10, in EtOH (100 mL) was added hydrazine hydrate (7.7 g, 150 mmol). The reaction mixture was stirred at reflux for 3 h, after which, the reaction mixture was concentrated in vacuo. To the resulting residue, water was added and the mixture extracted with EtOAc. The organic layer was washed with water, saturated NaCl solution, dried over Na₂SO₄ and concentrated in vacuo to afford intermediate 87 (7.0 g, yield 49%) as a white solid.

8-Chloro-N′-(2,5-dichloro-4-methoxybenzoyl)-6-(trifluoromethyl)imidazo[1,2-a]pyridine-2-carbohydrazide (88). To a stirred solution of intermediate 82 (4.5 g, 20 mmol) in DMF (45 mL) was added intermediate 87 (6.5 g, 23 mmol), EDCI.HCl (5.1 g, 27 mmol) and HOBT (2.75 g, 20.4 mmol). The reaction mixture was stirred at room temperature for 1 h, followed by heating to 100° C. for 12 h. The reaction mixture was quenched with water and extracted with EtOAc and the organic layer was washed with 5% K₂CO₃, 1 N HCl, water, saturated NaCl solution, dried over Na₂SO₄ and concentrated in vacuo to afford intermediate 88 (4.5 g, 46% yield)as a brown solid.

2-(8-Chloro-6-(trifluoromethyl)imidazo[1,2-a]pyridin-2-yl)-5-(2,5-dichloro-4-methoxyphenyl)-1,3,4-thiadiazole (89). To a stirred solution of intermediate 88 (4.5 g, 9.3 mmol) in toluene (50 mL) was added Lawesson's reagent (4.92 g, 12.2 mmol) and pyridine (2 mL). The reaction mixture was stirred at reflux for 2 h, then concentrated in vacuo. To the resulting residue, pyridine (15 mL) and P₂S₅ (8.3 g, 37 mmol) was added and the mixture heated to reflux for 2 h. The pyridine was concentrated and water added to the resulting residue. The resulting solid was filtered and washed with acetone to afford intermediate 89 (4 g, 90% yield) as a light yellowish solid.

2,5-Dichloro-4-(5-(8-chloro-6-(trifluoromethyl)imidazo[1,2-a]pyridin-2-yl)-1,3,4-thiadiazol-2-yl)phenol. To a stirred solution of intermediate 89 (3 g, 6.2 mmol) in DCM (50 mL) was added AlCl₃ (4.17 g 31.3 mmol) and EtSH (1.94 g, 31.2 mmol) dropwise at 0° C. The reaction mixture was stirred at room temperature for 12 h. The reaction mixture was quenched with ice water and the resulting solid was filtered and thoroughly dried to afford the title compound (2.3 g, 79% yield) as an off-white solid. ¹H NMR (400 MHz, DMSO-d₆) δ 11.6 (s, 1H, —OH), 9.3 (s, 1H), 8.9 (s, 1H), 8.3 (s, 1H), 8.0 (s, 1H), 7.2 (s, 1H); MS (EI) for C₁₆H₆Cl₃F₃N₄OS, found 465 (MH+).

Using the same or analogous synthetic techniques in Example 18 and substituting with appropriate reagents (prepared as described herein or as known to one of ordinary skill in the art), 5-chloro-4-{5-[8-chloro-6-(trifluoromethyl)imidazo[1,2-a]pyridin-2-yl]-1,3,4-thiadiazol-2-yl}-2-fluorophenol was prepared. ¹H-NMR (400 MHz, DMSO-d₆) δ 11.2 (s, 1H), 9.3 (s, 1H), 8.9 (s, 1H), 8.1 (d, 1H), 8.0 (s, 1H), 7.2 (d, 1H); MS (EI) for C₁₆H₆Cl₂F₄N₄OS, found 448.8 (MH+).

### Example 19

### 3-(2,5-Dichloro-4-(3-(8-chloro-6-(trifluoromethyl)imidazo[1,2-a]pyridin-2-yl)-1,2,4-oxadiazol-5-yl)phenyl)propanoic acid

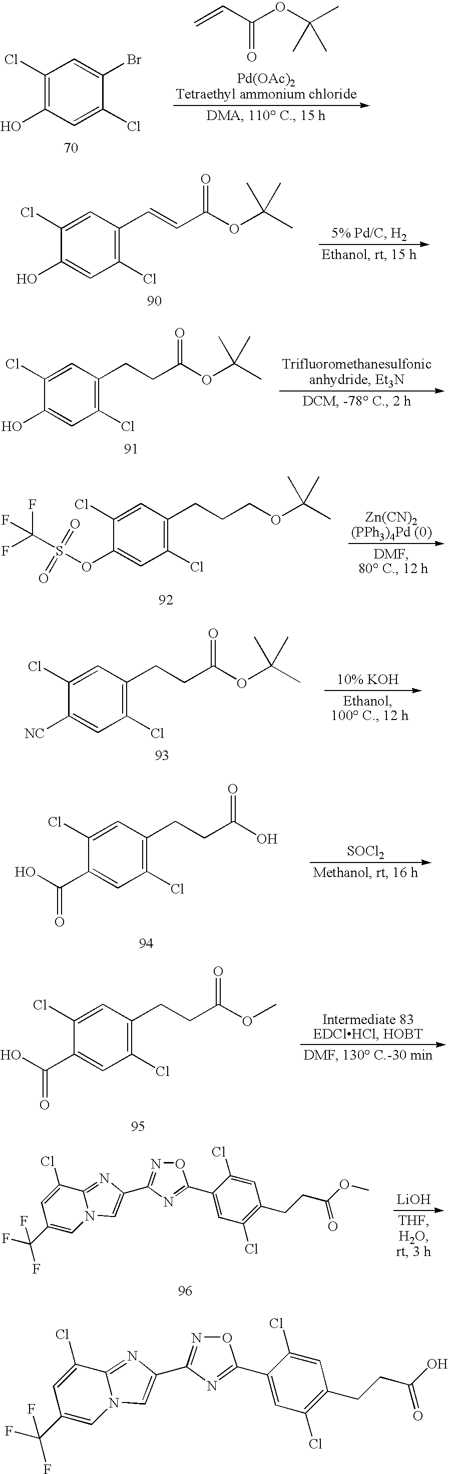

tert-Butyl 3-(2,5-dichloro-4-hydroxyphenyl)acrylate (90). To a stirred solution of 4-bromo-2,5-dichlorophenol 70 (10.0 g, 41.3 mmol) in dimethylacetamide (100 mL) was added tent-butyl acrylate (6.38 g, 49.7 mmol) and reaction was degassed with argon gas. To this solution, Pd(OAc)₂ (185 mg, 0.829 mmol) and tetraethylammonium chloride (6.87 g, 41.5 mol) was added and the reaction mixture was stirred at 110° C. for 15 h. After completion, the reaction mixture was quenched with ice cold water and extracted with ether. The combined organic layers were washed with water, saturated NaCl solution, dried over Na₂SO₄ and concentrated in vacuo. The crude compound was purified by column chromatography to afford the title intermediate 90 as a yellow solid (4.1 g, yield 34%).

tert-Butyl 3-(2,5-dichloro-4-hydroxyphenyl)propanoate (91). To a stirred solution of intermediate 90 (4 g, 14 mmol) in EtOH (20 mL) was added 5% Pd/C (400 mg) and the reaction was stirred under hydrogen atmosphere for 15 h. After completion, the reaction was filtered and the filtrate was concentrated in vacuo to afford the title intermediate 91 as a white solid (3.2 g, yield 79%).

tert-Butyl 3-(2,5-dichloro-4-(trifluoromethylsulfonyl)phenyl)propanoate (92). To a stirred solution of intermediate 91 (3.0 g, 10 mmol) in dichloromethane (30 mL) was added triethylamine (3.12 g, 30.9 mmol) and the reaction was cooled to −78° C. Trifluoromethanesulfonic anhydride (5.81 g, 20.6 mmol) was added slowly over a period of 20 min and the reaction mixture was stirred for 2 h at −78° C. After completion, the reaction mixture was diluted with dichloromethane and neutralized with saturated sodium bicarbonate solution. The organic phase was separated, washed with water, dried over Na₂SO₄ and concentrated to afford the title intermediate 92 as a solid (3.5 g, yield 83%).

tert-Butyl 3-(2,5-dichloro-4-cyanophenyl)propanoate (93). To a stirred degassed solution of intermediate 92 (6.0 g, 14 mmol) in dimethylformamide (30 mL) was added zinc cyanide (3.01 g, 16.9 mmol) followed by tetrakis(triphenylphosphine) palladium (0) (1.63 g, 1.41 mmol). The reaction mixture was stirred 80° C. for 12 h. After completion, the reaction mixture was concentrated in vacuo and diluted with EtOAc. The organic phase was washed with water, saturated NaCl solution, dried over Na₂SO₄ and concentrated. The crude compound was purified by column chromatography to afford the title intermediate 93 as a white solid (3.1 g, yield 73%).

4-(2-Carboxyethyl)-2,5-dichlorobenzoic acid (94). To a stirred solution of intermediate 93 (3 g, 10 mmol) in EtOH (5 mL) was added 10% KOH solution (30 mL) and the reaction was stirred at 100° C. for 12 h. After completion, the reaction mixture was neutralized with 2 N HCl and extracted with EtOAc. The organic layer was washed with water, saturated NaCl solution, dried over Na₂SO₄ and concentrated to afford the title intermediate 94 as a white solid (1.7 g, 65% yield).

2,5-Dichloro-4-(3-methoxy-3-oxopropyl)benzoic acid (95). To a stirred solution of intermediate 94 (1.0 g, 3.8 mmol) in MeOH (10 mL) was added thionyl chloride (22.6 mg, 0.189 mmol) at 0° C. After addition was complete, the reaction was stirred at room temperature for 16 h. After completion, the reaction mixture was concentrated in vacuo and the residue obtained was stirred with ether. The solid obtained was collected by filtration, washed well with ether and dried completely to afford the title intermediate 95 as a white solid (0.780 g, 75% yield).

Methyl 3-(2,5-dichloro-4-(3-(8-chloro-6-(trifluoromethyl)imidazo[1,2-a]pyridine-2-yl)-1,2,4-oxadiazol-5-yl)phenyl)propanoate (96). To a stirred solution of acid 95 (0.420 g, 1.51 mmol) in DMF (5 ml) was added EDCI.HCl (0.289 g, 1.51 mmol) followed by HOBT (0.204 mg, 1.51 mmol). After stirring for 20 min, hydroxyimidate 83 (0.325 g, 1.17 mmol) was added and the reaction was stirred at room temperature for 1 h and then at 130° C. for 30 min. After completion, the reaction mixture was concentrated in vacuo and the residue obtained was dissolved in EtOAc. The organic phase was washed with water, saturated NaCl solution, dried over Na₂SO₄ and concentrated. The residue was purified by column chromatography (10% EtOAc/hexane as eluent) to afford the title compound 96 as a yellow solid (0.270 g, 44.5% yield).

3-(2,5-Dichloro-4-(3-(8-chloro-6-(trifluoromethyl)imidazo[1,2-a]pyridin-2-yl)-1,2,4-oxadiazol-5-yl)phenyl)propanoic acid. To a stirred solution of intermediate 96 (0.270 g, 0.519 mmol) in THF:water (10 mL) was added lithium hydroxide (0.0546 g, 1.3 mmol) and the mixture stirred at room temperature for 3 h. After completion, the solvent was removed in vacuo and the resulting residue acidified with acetic acid at 0° C. The precipitated solid was collected by filtration, washed well with water and dried completely to afford the title compound as a white solid (0.06 g, 22% yield). ¹H NMR (400 MHz, DMSO-d₆) δ 9.30 (s, 1H), 8.90 (s, 1H), 8.25 (s, 1H), 8.00 (s, 1H), 7.80 (s, 1H), 3.00 (m, 2H), 2.65 (m, 2H); MS (EI) for C₁₉H₁₀Cl₃F₃N₄O₃, found 506 (MH+).

The following compounds were prepared using the same or analogous synthetic techniques in Example 19 and substituting with appropriate reagents, which were commercially available or were prepared using procedures herein or procedures known to one of ordinary skill in the art.

3-{5-Chloro-4-[3-(8-chloro-6-iodoimidazo[1,2-a]pyridin-2-yl)-1,2,4-oxadiazol-5-yl]-2-fluorophenyl}propanoic acid. MS (EI) for C₁₈H₁₀Cl₂FIN₄O₃, found 547 (MH+).

3-(2,5-Dichloro-4-{3-[8-chloro-6-(trifluoromethyl)imidazo[1,2-a]pyridin-2-yl]-1,2,4-oxadiazol-5-yl}phenyl)-2-methylpropanoic acid. MS (EI) for C₂₀H₁₂Cl₃F₃N₄O₃, found 519 (MH+).

8-Chloro-2-[5-(2,6-difluorophenyl)-1,2,4-oxadiazol-3-yl]-6-(trifluoromethyl)imidazo[1,2-a]pyridine. ¹H-NMR (400 MHz, CD3Cl) δ 8.55 (s, 1H), 8.45 (s, 1H), 7.50 (s, 1H), 7.40 (m, 1H), 6.80 (d, 1H), 6.65 (t, 1H).

8-Chloro-2-[5-(2-chlorophenyl)-1,2,4-oxadiazol-3-yl]-6-(trifluoromethyl)imidazo[1,2-a]pyridine. MS (EI) for C₁₆H₇Cl₂F₃N₄O, found 398.9 (MH+).

8-Chloro-2-[5-(2-fluorophenyl)-1,2,4-oxadiazol-3-yl]-6-(trifluoromethypimidazo[1,2-a]pyridine. MS (EI) for C₁₆H₇ClF₄N₄O, found 382.9 (MH+).

1-[(4-{3-[8-Chloro-6-(trifluoromethyl)imidazo[1,2-a]pyridin-2-yl]-1,2,4-oxadiazol-5-yl}phenyl)methyl]azetidine-3-carboxylic acid. MS (EI) for C₂₁H₁₅ClF₃N₅O₃, found 478.2 (MH+).

N-(5-Chloro-4-{3-[8-chloro-6-(trifluoromethyl)imidazo[1,2-a]pyridin-2-yl]-1,2,4-oxadiazol-5-yl}-2-fluorophenyl)methanesulfonamide. MS (EI) for C₁₇H₉Cl₂F₄N₅O₃S, found 510 (MH+).

3-(2-Chloro-4-{3-[8-chloro-6-(trifluoromethyl)imidazo[1,2-a]pyridin-2-yl]-1,2,4-oxadiazol-5-yl}phenyl)propanoic acid. MS (EI) for C₁₉H₁₁Cl₂F₃N₄O₃, found 471 (MH+).

3-(4-{3-[8-Bromo-6-(trifluoromethyl)imidazo[1,2-a]pyridin-2-yl]-1,2,4-oxadiazol-5-yl}-5-chloro-2-fluorophenyl)propanoic acid. MS (EI) for C₁₉H₁₀BrClF₄N₄O₃, found 534.7 (MH+).

3-(2,6-Dichloro-4-{3-[8-chloro-6-(trifluoromethyl)imidazo[1,2-a]pyridin-2-yl]-1,2,4-oxadiazol-5-yl}phenyl)propanoic acid. MS (EI) for C₁₉H₁₀Cl₃F₃N₄O₃, found 505 (MH+).

N-(2-Chloro-4-{3-[8-chloro-6-(trifluoromethyl)imidazo[1,2-a]pyridin-2-yl]-1,2,4-oxadiazol-5-yl}-6-fluorophenyl)methanesulfonamide. ¹H-NMR (400 MHz, DMSO-d₆) δ 10.00 (s, 1H), 9.35 (s, 1H), 8.87 (s, 1H), 8.22 (s, 1H), 8.18 (m, 1H), 8.00 (s, 1H), 3.20 (s, 3H); MS (EI) for C₁₇H₉Cl₂F₄N₅O₃S, found 510 (MH+).

2-(4-{3-[8-Chloro-6-(trifluoromethyl)imidazo[1,2-a]pyridin-2-yl]-1,2,4-oxadiazol-5-yl}-2-fluoro-5-methylphenyl)cyclopropanecarboxylic acid. MS (EI) for C₂₁H₁₃ClF₄N₄O₃, found 481 (MH+).

3-(5-Chloro-4-{3-[8-chloro-6-(trifluoromethyl)imidazo[1,2-a]pyridin-2-yl]-1,2,4-oxadiazol-5-yl}-2-fluorophenyl)propanoic acid. MS (EI) for C₁₉H₁₀Cl₂F₄N₄O₃, found 489 (MH+).

8-Chloro-2-[5-(2-chloro-4-fluorophenyl)-1,2,4-oxadiazol-3-yl]-6-(trifluoromethyl)imidazo[1,2-a]pyridine. ¹H NMR (400 MHz, CDCl₃) δ 8.64-8.60 (m, 1H), 8.58 (s, 1H), 8.11 (dd, J=8.8, 6.0, 1H), 7.75 (t, J=4.5, 1H), 7.32 (dd, J=8.5, 2.5, 1H), 7.15 (ddd, J=8.8, 7.7, 2.6, 1H). MS (EI) for C₁₆H₆Cl₂F₄N₄O, found 417 (MH+).

2-(2,5-Dichloro-4-{3-[8-chloro-6-(trifluoromethyl)imidazo[1,2-a]pyridin-2-yl]-1,2,4-oxadiazol-5-yl}phenyl)cyclopropanecarboxylic acid. MS (EI) for C₂₀H₁₀Cl₃F₃N₄O₃, found 517 (MH+).

3-(2-Chloro-4-{3-[8-chloro-6-(trifluoromethyl)imidazo[1,2-a]pyridin-2-yl]-1,2,4-oxadiazol-5-yl}-5-methylphenyl)propanoic acid. MS (EI) for C₂₀1H₁₃Cl₂F₃N₄O₃, found 484.9 (MH+).

3-{2-chloro-4-[3-(8-chloro-6-iodoimidazo[1,2-a]pyridin-2-yl)-1,2,4-oxadiazol-5-yl]-5-methylphenyl}propanoic acid. MS (EI) for C₁₉H₁₃Cl₂IN₄O₃, found 542.8 (MH+).

### Example 20

### 3-(2,5-Dichloro-4-(5-(8-chloro-6-(trifluoromethyl)-1H-imidazo[1,2-a]pyridin-2-yl)-1,2,4-oxadiazol-3-yl)phenyl)propanoic acid

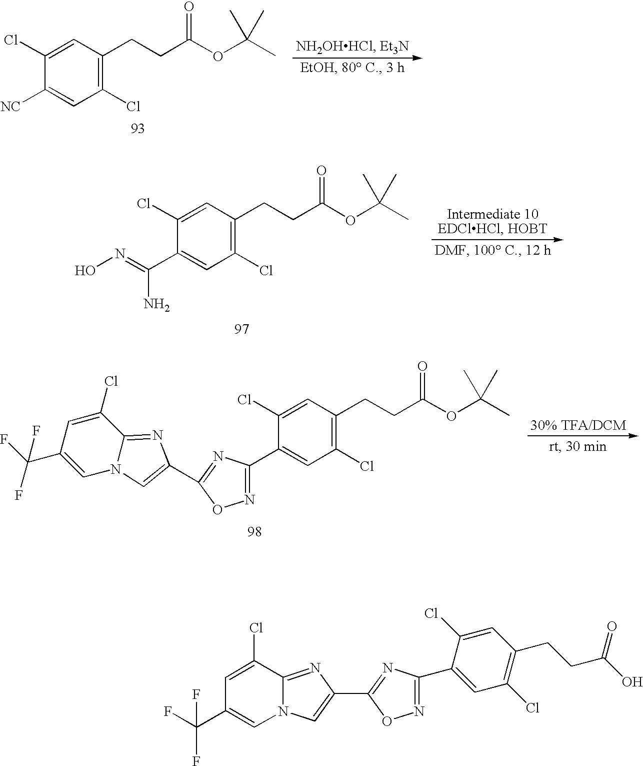

tert-Butyl 3-(4-amidino-2,5-dichlorophenyl)propanoate (97). To a stirred solution of hydroxylamine hydrochloride (2.07 g, 30 mmol) in EtOH (15 mL) was added triethylamine (4.8 mL, 35 mmol) and the mixture stirred at room temperature for 30 min. Intermediate 93 (1.5 g, 5 mmol) in EtOH (5 mL) was added and the reaction mixture was stirred at 80° C. for 3 h. After completion, the reaction mixture was concentrated in vacuo and diluted with EtOAc (30 mL). The organic phase was washed with water, saturated NaCl solution, dried over Na₂SO₄ and concentrated in vacuo to afford hydroxyimidate 97 as a yellow solid (1.6 g, 94% yield).

tert-Butyl-3-(2,5-dichloro-4-(5-(8-chloro-6-(trifluoromethyl)-imidazo[1,2-a]pyridin-2-yl)-1,2,4-oxadiazol-3-yl)phenyl)propanoate (98). To a stirred solution of Intermediate 10 (1.52 g, 5.67 mmol) in DMF (15 mL) was added EDCl.HCl (1.10 g, 5.78 mmol) followed by addition of HOBT (0.78 g, 5.8 mmol). After 30 min, intermediate 97 (1.6 g, 4.8 mmol) was added and the reaction was stirred at 100° C. for 12 h. After completion, the reaction mixture was concentrated in vacuo and the crude compound obtained was purified by column chromatography (15% EtOAc/hexane as eluent) to afford the title intermediate 98 as an off-white solid (1.2 g, 38% yield).

3-(2,5-dichloro-4-(5-(8-chloro-6-(trifluoromethyl)-1H-imidazo[1,2-a]pyridin-2-yl)-1,2,4-oxadiazol-3-yl)phenyl)propanoic acid. A solution of intermediate 98 (0.6 g, 1.14 mmol) in 30% TFA/DCM (10 mL) was stirred at room temperature for 30 min. After completion, the reaction mixture was concentrated in vacuo and the residue obtained was triturated with diethyl ether and iPrOH to afford the title compound as a white solid (0.350 g, 61% yield). ¹H NMR (400 MHz, DMSO-d₆) δ 12.35 (br s, 1H, COOH), 9.35 (s, 1H), 9.10 (s, 1H), 8.10 (d, 2H), 7.75 (s, 1H), 3.00 (m, 2H), 2.70 (m, 2H); MS (EI) for C₁₉H₁₀Cl₃F₃N₄O₃, found 505 (MH+).

### Example 21

### N-(3-Chloro-4-(5-(8-chloro-6-(trifluoromethyl)imidazo[1,2-a]pyridine-2-yl)-1,3,4-thiadiazol-2-yl)Phynyl)methanesulfonamide

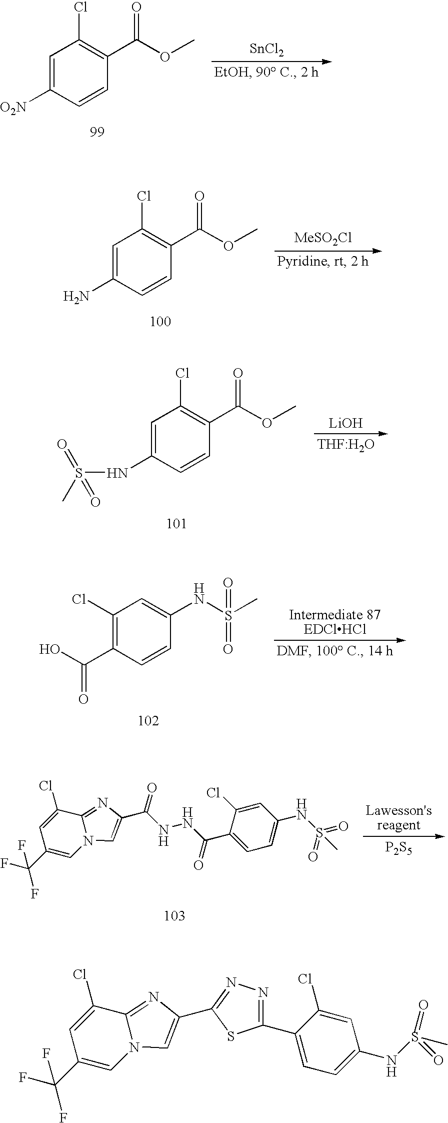

Methyl-4-amino-2-chlorobenzoate (100). To a stirred solution of methyl-2-chloro-4-nitro benzoate 99 (0.50 g, 2.3 mmol) in EtOH was added stannous chloride (2.62 g, 11.6 mmol) at 0° C. and the resulting mixture was stirred at 90° C. for 2 h. After completion, the reaction mixture was allowed to cool to room temperature and concentrated in vacuo. 1 M NaOH (20 mL) and EtOAc (30 mL) was added to the residue and the resulting mixture filtered through Celite. The filtrate was extracted with EtOAc (3×25 mL) and the combined organic layers were dried over Na₂SO₄ and concentrated. The crude compound was purified by column chromatography, eluting with 50% EtOAc/hexane, to afford the title intermediate 100 as a yellow solid (0.431 g, 100% yield).

Methyl-2-chloro-4-(methylsulfonamido) benzoate (101). To a stirred solution of methyl-2-chloro-4-amino benzoate 100 (0.457 g, 2.45 mmol) in DCM cooled to 0° C. was added pyridine (2 mL) followed by dropwise addition of methanesulphonyl chloride (0.2 mL, 2.5 mmol). After addition was complete, the reaction was allowed to warm to room temperature and stirred for 2 h. After completion, the reaction mixture was concentrated in vacuo. 1 N HCl (5 mL) was added to the residue and the mixture extracted with EtOAc (10 mL). The organic phase was dried over Na₂SO₄ and concentrated under high vacuum. The crude compound was purified by column chromatography to afford the title intermediate 101 as a solid (0.54 g, 84% yield).

2-Chloro-4-(methylsulfonamido)benzoic acid (102). To a stirred solution of methyl-2-chloro-4-(methylsulfonamido)benzoate 101 (0.54 g, 2.04 mmol) in THF:water (1:1, 10 mL) was added lithium hydroxide (0.171 g, 4.09 mmol) and the mixture stirred at room temperature for 3 h and then heated to 45° C. for 1 h. After completion, the reaction mixture was concentrated in vacuo and the aqueous phase was acidified by dropwise addition of acetic acid at 0° C. The reaction mixture was extracted with EtOAc, dried over Na₂SO₄ and concentrated in vacuo to give intermediate 102 as a white solid (0.48 g, 94% yield).

N-(3-Chloro-4-(2-(8-chloro-6-(trifluoromethyl)imidazol(1,2-a)pyridine-2-carbonyl)hydrazine carbonyl)phynyl)methanesulfonamide (103). To a stirred solution of acid 102 (1.0 g, 4.0 mmol) in DMF (6 mL) was added EDCI.HCl (0.780 g, 4.07 mmol) followed by HOBT (0.560 g, 4.14 mmol). After stirring the reaction mixture for 15 min, intermediate 87 (1.34 g, 4.33 mmol), prepared as described above in Example 18, was added and stirring continued at room temperature. After 1 h, the reaction mixture was heated to 100° C. for 14 h. After completion, the reaction mixture was concentrated in vacuo and to the resulting residue was dissolved in EtOAc which was washed with water, saturated NaCl, dried over Na₂SO₄ and concentrated. The crude compound was purified by column chromatography to afford the title intermediate 103 as a yellow solid (0.306 g, 15% yield).

N-(3-Chloro-4-{5-[8-chloro-6-(trifluoromethyl)imidazo[1,2-a]pyridin-2-yl]-1,3,4-thiadiazol-2-yl}phenyl)methanesulfonamide. To a stirred solution of intermediate 103 (2.27 g, 4.45 mmol) in toluene (20 mL) was added pyridine (0.825 mL, 10 mmol) followed by Lawesson's reagent (2.367 g, 5.79 mmol) and the reaction mixture was stirred at 125° C. for 4 h. After completion, the reaction mixture was cooled to room temperature and concentrated under reduced pressure. The solid obtained was dissolved in pyridine (30 mL) and phosphorous pentasulfide (3.92 g, 17.6 mmol) was added and the reaction mixture stirred at 110° C. for 2 h. After completion, the reaction mixture was cooled to 0° C., water (25 mL) was added and the resulting mixture extracted with EtOAc (3×20 mL). The combined organic layers were dried over Na₂SO₄ and concentrated under high vacuum to afford crude compound which was further purified by recrystallization using N-methyl pyrolidinone and water to afford the title compound as a pink solid (0.863 g, 38.2% yield). ¹H NMR (400 MHz, DMSO-d₆) δ 10.55 (s, 1H), 9.28 (s, 1H), 8.86 (s, 1H), 8.25 (d, 1H), 8.0 (s, 1H), 7.48 (s, 1H), 7.4 (d, 1H), 3.20 (s, 3H); MS (EI) for C₁₇H₁₀Cl₂F₃N₅O₂S₂, found 508 (MH+).

### Example 22

### 1-Amino-3-(5-chloro-4-(5-(8-chloro-6-(trifluoromethyl)imidazo[1,2-a]pyridin-2-yl)-1,3,4-thiadiazol-2-yl)-2-fluorophenoxy)propan-2-ol

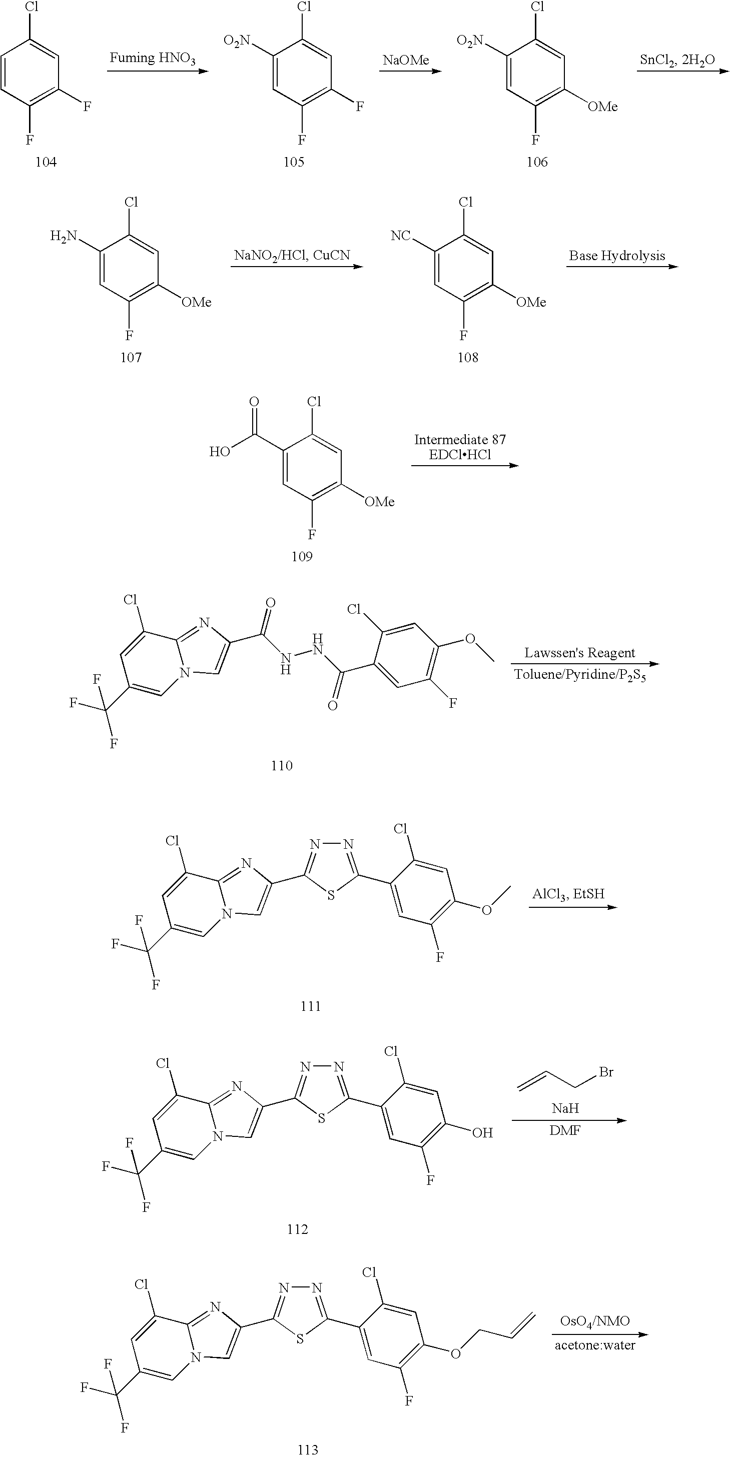

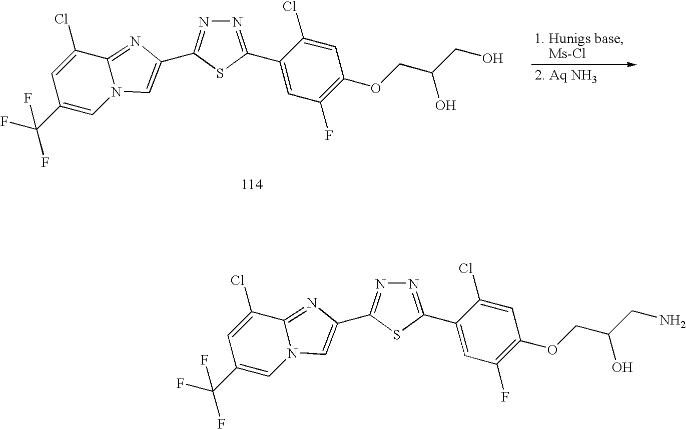

2-Chloro-4,5-difluoro nitrobenzene (105). Fuming nitric acid (50 mL) was added dropwise at 0° C. to 1-chloro-3,4-difluorobenzene 104 (25 g, 168 mmol) over a period of 1 h. After addition was complete, the reaction mixture was stirred at 0° C. for 15 min and allowed to warm to room temperature and stirred for 2 h. After completion, the reaction mixture was quenched with ice and extracted with diethyl ether. The combined organic layers were washed with water, saturated NaCl solution, dried over Na₂SO₄ and concentrated in vacuo to afford the title intermediate 105 as a liquid (32.0 g, 98.3% yield).

2-Chloro-4-methoxy-5-fluoro nitrobenzene (106). To a cooled solution of 0.5 M NaOCH₃ (8.93 g, 165 mmol, 320 mL MeOH) was added a solution of intermediate 105 (32 g, 165 mmol) in MeOH (32 mL) dropwise over a period of 30 min. After addition was complete, the reaction mixture was stirred at 0° C. for 30 min and then allowed to warm to room temperature and stirred for 2 h. After completion, the reaction mixture was quenched with ice and the precipitated solid was collected by filtration, washed well with water and dried completely to afford the title intermediate 106 as a white solid (30 g, 88% yield).

2-Chloro-4-methoxy-5-fluoro aniline (107). To a stirred solution of intermediate 106 (30 g, 146 mmol) in water (360 mL) was added SnCl₂.2H₂O (131.71 g, 584 mmol) followed by slow addition of concentrated HCl (300 mL) and the resulting mixture was stirred at 55° C. for 3 h. After completion, the reaction mixture was cooled to 0° C., quenched with ice, neutralized with 1N KOH solution and extracted with EtOAc. The combined organic layers were washed with saturated NaCl, dried over Na₂SO₄ and concentrated. The resulting residue was stirred in n-pentane, filtered and dried to afford the title intermediate 107 as a white solid (25 g, 97% yield).

2-Chloro-4-methoxy-5-fluoro benzonitrile (108). To a stirred solution of intermediate 107 (3.0 g, 17 mmol) in a mixture of water (15 mL) and concentrated HCl (9 mL) cooled to 0° C. was added a solution of sodium nitrite (1.173 g, 18 mmol) in water (3.5 mL) dropwise over a period of 20 min maintaining the reaction temperature at 0° C. After addition, the reaction mixture was stirred at 0° C. for 30 min. To a pre-cooled solution of copper (I) cyanide (15.3 g, 17 mmol) and sodium cyanide (8.3 g, 17 mmol) in water (81 mL) was gradually added the above described diazonium salt over a period of 50 min. The diazonium salt solution was maintained at 0° C. during the addition. The resulting mixture was stirred for 18 h at room temperature. The obtained precipitate was filtered, washed with water and dried, then dissolved in EtOAc and washed with water followed by saturated NaCl solution. The organic phase was dried over Na₂SO₄ and concentrated in vacuo. The crude compound was purified by column chromatography, eluting with 5-10% EtOAc/hexane, to afford the title intermediate 108 as a solid (2.40 g, 77.4% yield).

2-Chloro-4-methoxy-5-fluoro benzoic acid (109). To a stirred solution of intermediate 108 (0.615 g, 3.31 mmol) in EtOH (5 mL) was added 10% KOH solution (20 mL) and the resulting mixture was stirred at 100° C. for 12 h. After completion, the reaction mixture was neutralized with 1 N HCl and extracted with EtOAc. The combined organic layers were washed with water, saturated NaCl solution, dried over Na₂SO₄ and concentrated to afford the title intermediate 109 as a white solid (0.510, 75.1% yield).

8-Chloro-N′-(2-chloro-5-fluoro-4-methoxybenzoyl)-6-(trifluoromethyl)imidazo[1,2-a]pyridine-2-carbohydrazide (110). To a stirred solution of acid 109 (0.510 g, 2.5 mmol) in DMF (10 mL) was added EDCI.HCl (0.960 g, 5 mmol) followed by intermediate 87 (0.831 g, 3 mmol). The reaction was stirred at room temperature for 1 h and then at 100° C. for 14 h. After completion, the reaction mixture was concentrated in vacuo and the residue obtained was dissolved in EtOAc. The organic phase was washed with water, saturated NaCl solution, dried over Na₂SO₄ and concentrated. The crude compound was purified by column chromatography, eluting with 5-10% EtOAc/hexane, to afford the title intermediate 110 as a yellow solid (0.634 g, 55.13% yield).

2-(2-Chloro-5-fluoro-4-methoxyphenyl)-5-(8-chloro-6-(trifluoromethyl)imidazo[1,2-a]pyridin-2-yl)-1,3,4-thiadiazole (111). To a stirred solution of intermediate 110 (0.634 g, 1.36 mmol) in toluene (7 mL) was added pyridine (0.3 mL) and Lawesson's reagent (0.716 g, 1.36 mmol) and the reaction mixture was stirred at 120° C. for 4 h. The reaction mixture was concentrated under high vacuum. The resulting solid was dissolved in pyridine (7 mL) and phosphorous pentasulfide (1.21 g, 5 mmol) was added and the resulting mixture stirred at 120° C. for 4 h. After completion, the reaction mixture was cooled to 0° C. and quenched with water. The aqueous phase was extracted with EtOAc and the combined organic layers were dried over Na₂SO₄ and concentrated. The crude solid was purified by recrystallization using EtOAc to afford the title intermediate 111 as a pink solid (0.250 g, 39.6% yield).

5-Chloro-4-(5-(8-chloro-6-(trifluoromethyl)imidazo[1,2-a]pyridin-2-yl)-1,3,4-thiadiazol-2-yl)-2-fluorophenol (112). To a cold solution of intermediate 111 (0.250 g, 0.53 mmol) in DCM (7 mL) was added AlCl₃ (0.215 g, 1.6 mmol) in small portions such that the reaction temperature was maintained below 10° C. The light brown suspension was stirred for 10 min and then EtSH (0.100 g, 1.6 mmol) was added dropwise at such a rate that the reaction temperature was maintained below 5° C. After 2.5 h of stirring below 10° C., the reaction mixture was slowly poured into ice water with strong agitation. The organic layer was separated, and the aqueous layer was extracted with DCM. The combined DCM layers were washed with water, saturated NaCl, dried over Na₂SO₄ and concentrated to afford the title intermediate 112 as a light yellow solid (0.170 g, 70.2% yield).

2-(4-(Allyloxy)-2-chloro-5-fluorophenyl)-5-(8-chloro-6-(trifluoromethyl)imidazo[1,2-a]pyridin-2-yl)-1,3,4-thiadiazole (113). To a stirred solution of intermediate 112 (0.170 g, 0.37 mmol) in dry DMF (4 mL) was added K₂CO₃ (0.209 g, 1.51 mmol) at 0° C. followed by dropwise addition of allyl bromide (0.13 mL, 1.5 mmol). After addition, the reaction was stirred at 80° C. for 3 h. After completion, the reaction was diluted with water and extracted with EtOAc. The combined EtOAc extracts were washed with water, dried over Na₂SO₄ and concentrated to give the title intermediate 113 as a white solid (0.165 g, 89.2% yield).

3-(5-Chloro-4-(5-(8-chloro-6-(trifluoromethyl)imidazo[1,2-a]pyridin-2-yl)-1,3,4-thiadiazol-2-yl)-2-fluorophenoxy)propane-1,2-diol (114). To a stirred solution of intermediate 113 (0.301 g, 0.63 mol) in a mixture of acetone:water (6 mL/0.5 mL) was added OsO₄ (0.2 mL, 0.1 M solution in toluene) and NMO (2 mL). After addition, the reaction was stirred at room temperature overnight. After completion, the reaction mixture was quenched with saturated sodium sulfite solution and stirring was continued for an additional 45 min. The suspended solid was collected by filtration, washed well with water, ether and dried completely to afford the title intermediate 114 as a white solid (0.225 g, 70% yield).

1-Amino-3-(5-chloro-4-(5-(8-chloro-6-(trifluoromethyl)imidazo[1,2-a]pyridin-2-yl)-1,3,4-thiadiazol-2-yl)-2-fluorophenoxy)propan-2-ol. To a stirred solution of diol 114 (0.258 g, 0.49 mmol) in THF (5 mL) was added Hunig's base (0.17 mL, 0.98 mmol) followed by methanesulfonylchloride (0.03 mL g 0.49 mmol) at 0° C. The reaction mixture was stirred at room temperature for 16 h. After completion, the reaction mixture was concentrated under vacuum. To the residue, 7 M NH₃ in MeOH (5 mL) was added and the resulting mixture heated in a sealed tube at 60° C. for 12 h. After completion, the reaction mixture was concentrated. The crude product was purified by preparative HPLC to afford the title compound as a white solid (10 mg, 4.9%) yield. ¹H-NMR (400 MHz, DMSO-d₆) δ 9.28 (s, 1H), 8.90 (s, 1H), 8.20 (d, 1H), 8.0 (s, 1H), 7.6 (d, 1H), 7.58 (br s, 2H), 5.90 (d, 1H), 4.02-4.22 (m, 3H), 3.20 (m, 2H); MS (EI) for C₁₉H₁₃Cl₂F₄N₅O₂S, found 522 (MH+).

The following compounds were prepared using the same or analogous synthetic techniques in Example 22 and substituting with appropriate reagents, which were commercially available or were prepared using procedures herein or procedures known to one of ordinary skill in the art. For a number of the following compounds, the last one or two steps were omitted as applicable to yield the desired product. A person of ordinary skill in the art would be able to readily ascertain which steps were omitted.

(2S)-3-[(2,5-Dichloro-4-{5-[8-chloro-6-(trifluoromethyl)imidazo[1,2-a]pyridin-2-yl]-1,3,4-thiadiazol-2-yl}phenyl)oxy]propane-1,2-diol. ¹H-NMR (400 MHz, DMSO-d₆) δ 9.33 (s, 1H), 8.92 (s, 1H), 8.35 (s, 1H), 8.01 (s, 1H), 7.58 (s, 1H), 5.12-5.11 (d, 1H), 4.79-4.75 (t, 1H), 4.27-4.23 (dd, 1H), 4.18-4.14 (dd, 1H), 3.88-3.84 (m, 1H), 3.51-3.48 (t, 2H); MS (EI) for C₁₉H₁₂Cl₃F₃N₄O₃S, found 541 (MH+).

2-[(3-Chloro-4-{5-[8-chloro-6-(trifluoromethyl)imidazo[1,2-a]pyridin-2-yl]-1,3,4-thiadiazol-2-yl}phenyl)oxy]ethanamine. MS (EI) for C₁₈H₁₂Cl₂F₃N₅0S, found 474.0 (MH+).

3-[(3-Chloro-4-{5-[8-chloro-6-(trifluoromethyl)imidazo[1,2-a]pyridin-2-yl]-1,3,4-thiadiazol-2-yl}phenyl)oxy]propane-1,2-diol. MS (EI) for C₁₉H₁₃Cl₂F₃N₄O₃S, found 505 (MH+).

3-[(4-{5-[8-Chloro-6-(trifluoromethyl)imidazo[1,2-a]pyridin-2-yl]-1,3,4-thiadiazol-2-yl}-2,6-dimethylphenyl)oxy]propane-1,2-diol. MS (EI) for C₂₁H₁₈ClF₃N₄O₃S, found 499 (MH+).

3-[(5-Chloro-4-{5-[8-chloro-6-(trifluoromethyl)imidazo[1,2-a]pyridin-2-yl]-1,3,4-thiadiazol-2-yl}-2-fluorophenyl)oxy]propane-1,2-diol. MS (EI) for C₁₉H₁₂Cl₂F₄N₄O₃S, found 523 (MH+).

2-[(5-Chloro-4-{5-[8-chloro-6-(trifluoromethyl)imidazo[1,2-a]pyridin-2-yl]-1,3,4-thiadiazol-2-yl}-2-fluorophenyl)oxy]propan-1-ol. MS (EI) for C₁₉H₁₂Cl₂F₄N₄O₂S, found 507 (MH+).

2-[(5-Chloro-4-{5-[8-chloro-6-(trifluoromethyl)imidazo[1,2-a]pyridin-2-yl]-1,3,4-thiadiazol-2-yl}-2-fluorophenyl)oxy]propanoic acid. ¹H-NMR (400 MHz, DMSO-d₆) δ 9.31 (s, 1H), 8.89 (s, 1H), 8.15 (d, 1H), 7.95 (s, 1H), 7.44 (d, 1H), 5.23 (m, 1H), 1.58 (d, 3H); MS (EI) for C₁₉H₁₀Cl₂F₄N₄O₃S, found 521 (MH+).

2-[(2,5-Dichloro-4-{5-[8-chloro-6-(trifluoromethyl)imidazo[1,2-a]pyridin-2-yl]-1,3,4-thiadiazol-2-yl}phenyl)oxy]propan-1-ol. MS (EI) for C₁₉H₁₂Cl₃F₃N₄O₂S, found 523 (MH+).

2-[(3-Chloro-4-{5-[8-chloro-6-(trifluoromethyl)imidazo[1,2-a]pyridin-2-yl]-1,3,4-thiadiazol-2-yl}phenyl)oxy]ethanol. MS (EI) for C₁₈H₁₁Cl₂F₃N₄O₂S, found 475.0 (MH+).

1-amino-3-[(2,5-dichloro-4-{5-[8-chloro-6-(trifluoromethyl)imidazo[1,2-a]pyridin-2-yl]-1,3,4-thiadiazol-2-yl}phenyl)oxy]propan-2-ol. ¹H-NMR (400 MHz, DMSO-d₆) δ 9.4 (s, 1H), 8.8 (s, 1H), 8.3 (s, 1H), 7.9 (s, 1H), 7.5 (s, 1H), 5.1 (d, 1H), 4.7 (t, 1H), 4.2 (m, 2H), 3.8 (s, 2H), 3.4 (t, 2H). MS (EI) for C₁₉H₁₃Cl₃F₃N₅O₂S, found 538 (MH+).

### Example 23

### 2-{5-Chloro-4-[5-(8-chloro-6-trifluoromethyl-imidazo[1,2-a]pyridin-2-yl)-[1,2,4]oxadiazol-3-yl]-2-fluoro-phenoxy}-ethanol

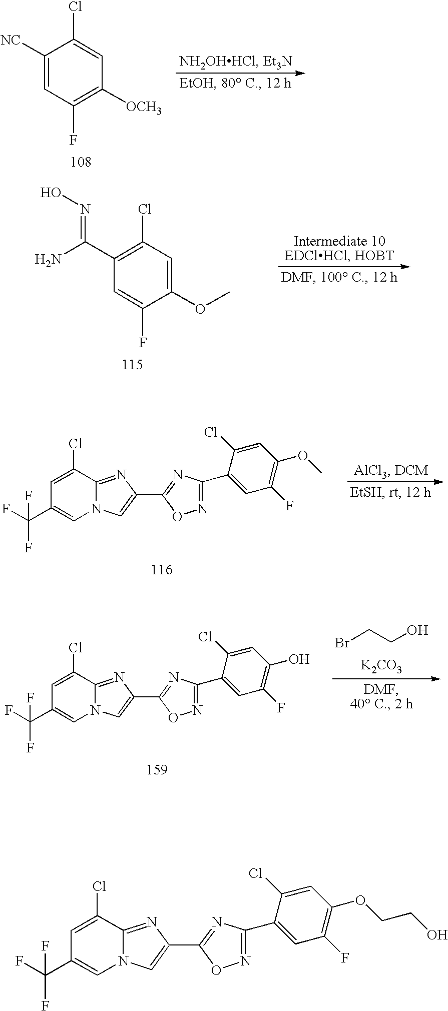

2-Chloro-4-methoxy-5-fluoro-N-hydroxy benzamidine (115). To a stirred solution of hydroxylamine hydrochloride (11.20 g, 161.3 mmol) in EtOH (50 mL) was added triethylamine (19.0 g, 188 mmol) followed by addition of intermediate 108 (5.0 g, 27 mmol). The resulting solution was stirred at 80° C. for 12 h. After completion, the reaction mixture was concentrated in vacuo and dissolved in EtOAc. The organic layer was washed with water (2×), saturated NaCl solution, dried over Na₂SO₄ and concentrated to afford the title intermediate 115 as a white solid (5.1 g, 87% yield).

8-Chloro-2-[3-(2-chloro-5-fluoro-4-methoxy-phenyl)41,2,4]oxadiazol-5-yl]-6-trifluoromethyl-imidazo[1,2-a]pyridine (116). To a stirred solution of Intermediate 10 (7.86 g, 29.7 mmol) in DMF (50 mL) was added EDCI.HCl (5.69 g, 29.8 mmol) followed by HOBT (4.006 g, 29.62 mmol) and hydroxyimidate 115 (5.0 g, 23 mmol). The resulting mixture was stirred at room temperature for 1 h and then at 100° C. for 12 h. After completion, the reaction mixture was concentrated in vacuo and dissolved in EtOAc. The organic layer was washed with saturated sodium bicarbonate, water, saturated NaCl solution, dried over Na₂SO₄ and concentrated. The residue was purified by column chromatography (10% EtOAc/hexane as eluent) to afford the title intermediate 116 as a yellow solid (2.5 g, 25% yield).

5-Chloro-4-[5-(8-chloro-6-trifluoromethyl-imidazo[1,2-a]pyridin-2-yl)-[1,2,4]oxadiazol-3-yl]-2-fluoro-phenol. To a stirred solution of intermediate 116 (2.4 g, 5.4 mmol) in DCM cooled to 0° C. was added AlCl₃ (3.57 g, 26.8 mmol) slowly over 20 min. The reaction mixture was stirred at 0° C. for 30 min and EtSH (1.68 gm, 27.0 mmol) was added slowly. The resulting reaction mixture was allowed to warm to room temperature and stirred for 12 h. After completion, the reaction mixture was quenched with ice water and extracted with EtOAc. The combined organic layers were washed with water, saturated NaCl solution, dried over Na₂SO₄ and concentrated. The residue was purified by column chromatography (10% EtOAc/hexane as eluent) to afford the title compound 159 as an off-white solid (0.7 g, 30% yield). ¹H-NMR (400 MHz, DMSO-d₆) δ 11.20 (s, 1H), 9.35 (s, 1H), 9.05 (s, 1H), 8.05 (s, 1H), 7.85 (d, 1H), 7.20 (d, 1H); MS (EI) for C₁₆H₆Cl₂F₄N₄O₂, found 433 (MH+).

2-{5-Chloro-4-[5-(8-chloro-6-trifluoromethyl-imidazo[1,2-a]pyridin-2-yl)-[1,2,4]oxadiazol-3-yl]-2-fluoro-phenoxy}-ethanol. To a stirred solution of compound 159 (0.70 g, 1.6 mmol) in DMF (5 mL) was added K₂CO₃ (0.669 g, 4.84 mmol) and 2-bromoethanol (0.707 g, 5.65 mmol). The reaction was stirred at 40° C. for 2 h. After completion, the reaction mixture was concentrated in vacuo and the residue was dissolved in EtOAc. The organic layer was washed with water, saturated NaCl solution, dried over Na₂SO₄ and concentrated. The crude compound was purified by preparative HPLC to afford the title compound as an off-white solid (0.120 g, 15.6% yield). ¹H-NMR (400 MHz, DMSO-d₆) δ 9.30 (s, 1H), 9.05 (s, 1H), 8.05 (s, 1H), 7.90 (d, 1H), 7.55 (d, 1H), 4.25 (m, 2H), 3.80 (m, 3H); MS (EI) for C₁₈H₁₀Cl₂F₄N₄O₃, found 476.8 (MH+).

The following compounds were prepared using the same or analogous synthetic techniques in Example 23 and substituting with appropriate reagents, which were commercially available or were prepared using procedures herein or procedures known to one of ordinary skill in the art.

(2S)-2-[(5-Chloro-4-{5-[8-chloro-6-(trifluoromethyl)imidazo[1,2-a]pyridin-2-yl]-1,2,4-oxadiazol-3-yl}-2-fluorophenyl)oxy]propan-1-ol. ¹H-NMR (400 MHz, DMSO-d₆) δ 9.34 (s, 1H), 9.05 (s, 1H), 8.06 (s, 1H), 7.89 (d, 1H), 7.62 (d, 1H), 5.00 (t, 1H), 4.74 (q, 1H), 3.58 (t, 2H), 1.28 (d, 3H); MS (EI) for C₁₉H₁₂Cl₂F₄N₄O₃, found 491.0 (MH+).

(2R)-2-[(5-Chloro-4-{5-[8-chloro-6-(trifluoromethyl)imidazo[1,2-a]pyridin-2-yl]-1,2,4-oxadiazol-3-yl}-2-fluorophenyl)oxy]propan-1-ol. ¹H-NMR (400 MHz, DMSO-d₆) δ 9.34 (m, 1H), 9.05 (s, 1H), 8.06 (dd, 1H), 7.90-7.87 (d, 1H), 7.63-7.61 (d, 1H), 5.02-5.00 (t, 1H), 4.75-4.72 (m, 1H), 3.60-3.57 (t, 2H), 1.29-1.27 (d, 3H); MS (EI) for C₁₉H₁₂Cl₂F₄N₄O₃, found 491 (MH+).

(2S)-2-[(4-{5-[8-Chloro-6-(trifluoromethyl)imidazo[1,2-a]pyridin-2-yl]-1,2,4-oxadiazol-3-yl}-2,6-dimethylphenyl)oxy]propan-1-ol. ¹H-NMR (400 MHz, DMSO-d₆) δ 9.34 (s, 1H), 9.03 (s, 1H), 8.06 (s, 1H), 7.79 (s, 1H), 5.76 (s, 1H), 4.90 (m, 1H), 3.59 (m, 1H), 3.51 (m, 1H), 2.34 (s, 6H), 1.20 (d, 3H); MS (EI) for C₂₁H₁₈ClF₃N₄O₃, found 467.1 (MH+).

(2R)-2-[(4-{5-[8-Chloro-6-(trifluoromethyl)imidazo[1,2-a]pyridin-2-yl]-1,2,4-oxadiazol-3-yl}-2,6-dimethylphenyl)oxy]propan-1-ol. ¹H-NMR (400 MHz, DMSO-d₆) δ 9.35 (s, 1H), 9.03 (s, 1H), 8.07 (s, 1H), 7.79 (s, 1H), 4.89 (t, 1H), 4.14 (q, 1H), 3.59 (m, 1H), 3.50 (m, 1H), 2.34 (s, 6H), 1.20 (d, 3H); MS (EI) for C₂₁H₁₈ClF₃N₄O₃, found 467.1 (MH+).

Ethyl 2-[(5-chloro-4-{5-[8-chloro-6-(trifluoromethyl)imidazo[1,2-a]pyridin-2-yl]-1,2,4-oxadiazol-3-yl}-2-fluorophenyl)oxy]propanoate. ¹H-NMR (400 MHz, DMSO-d₆) δ 9.35 (s, 1H), 9.06 (s, 1H), 8.08 (s, 1H), 7.96 (d, 1H), 7.50 (d, 1H), 5.40 (q, 1H), 4.20 (q, 2H), 1.59 (d, 3H), 1.20 (t, 3H); MS (EI) for C₂₁H₁₄Cl₂F₄N₄O₄, found 533.0 (MH+).

2-[(5-chloro-4-{5-[8-chloro-6-(trifluoromethyl)imidazo[1,2-a]pyridin-2-yl]-1,2,4-oxadiazol-3-yl}-2-fluorophenyl)oxy]propanoic acid. ¹H-NMR (400 MHz, DMSO-d₆) δ 9.34 (s, 1H), 9.03 (s, 1H), 8.05 (s, 1H), 7.90 (d, 1H), 7.32 (d, 1H), 7.10 (br s, 1H), 5.08 (s, 1H), 1.56 (d, 3H); MS (EI) for C₁₉H₁₀Cl₂F₄N₄O₄, found 505.0 (MH+).

2-[(5-Chloro-4-{5-[8-chloro-6-(trifluoromethyl)imidazo[1,2-a]pyridin-2-yl]-1,2,4-oxadiazol-3-yl}-2-fluorophenyl)oxy]propan-1-ol. MS (EI) for C₁₉H₁₂Cl₂F₄N₄O₃, found 491 (MH+).

8-Chloro-2-[3-(2,5-dichloro-4-{[2-(methyloxy)ethyl]oxy}phenyl)-1,2,4-oxadiazol-5-yl]-6-(trifluoromethyl)imidazo[1,2-a]pyridine. MS (EI) for C₁₉H₁₂Cl₃F₃N₄O₃, found 506.9 (MH+).

3-[(2,5-Dichloro-4-{5-[8-chloro-6-(trifluoromethyl)imidazo[1,2-a]pyridin-2-yl]-1,2,4-oxadiazol-3-yl}phenyl)oxy]propan-1-ol. MS (EI) for C₁₉H₁₂Cl₃F₃N₄O₃, found 506.7 (MH+).

2-{[(2,5-Dichloro-4-{5-[8-chloro-6-(trifluoromethyl)imidazo[1,2-a]pyridin-2-yl]-1,2,4-oxadiazol-3-yl}phenyl)oxy]methyl}propane-1,3-diol. MS (EI) for C₂₀H₁₄Cl₃F₃N₄O₄, found 536.9 (MH+).

2-[(4-{5-[8-Bromo-6-(trifluoromethyl)imidazo[1,2-a]pyridin-2-yl]-1,2,4-oxadiazol-3-yl}-2,5-dichlorophenyl)oxy]ethanol. MS (EI) for C₁₈H₁₀BrCl₂F₃N₄O₃, found 539 (MH+).

[(3-Chloro-4-{5-[8-chloro-6-(trifluoromethyl)imidazo[1,2-a]pyridin-2-yl]-1,2,4-oxadiazol-3-yl}phenyl)oxy]acetic acid. ¹H-NMR (400 MHz, DMSO-d₆) δ 9.32 (s, 1H), 9.05 (s, 1H), 8.03 (s, 1H), 7.91 (d, 1H), 7.08 (m, 1H), 6.99 (m, 1H), 4.31 (m, 2H), 1.56 (d, 3H).

2-[(3-Chloro-4-{5-[8-chloro-6-(trifluoromethyl)imidazo[1,2-a]pyridin-2-yl]-1,2,4-oxadiazol-3-yl}phenyl)oxy]-2-methylpropanoic acid. MS (EI) for C₂₀H₁₃Cl₂F₃N₄O₄, found 501 (MH+).

2-[(2-Chloro-4-{5-[8-chloro-6-(trifluoromethyl)imidazo[1,2-a]pyridin-2-yl]-1,2,4-oxadiazol-3-yl}-6-fluorophenyl)oxy]propan-1-ol. MS (EI) for C₁₉H₁₂Cl₂F₄N₄O₃, found 491 (MH+).

8-Chloro-2-{3-[2-chloro-5-fluoro-4-(pyrrolidin-3-yloxy)phenyl]-1,2,4-oxadiazol-5-yl}-6-(trifluoromethyl)imidazo[1,2-a]pyridine. MS (EI) for C₂₀H₁₃Cl₂F₄N₅O₂, found 502.0 (MH+).

(4S)-4-[(3-Chloro-4-{5-[8-chloro-6-(trifluoromethyl)imidazo[1,2-a]pyridin-2-yl]-1,2,4-oxadiazol-3-yl}phenyl)oxy]-L-proline. MS (EI) for C₂₁H₁₄Cl₂F₃N₅O₄, found 528 (MH+).

(4S)-4-[(3-Chloro-4-{5-[8-chloro-6-(trifluoromethyl)imidazo[1,2-a]pyridin-2-yl]-1,2,4-oxadiazol-3-yl}phenyl)oxy]-D-proline. MS (EI) for C₂₁H₁₄Cl₂F₃N₅O₄, found 528 (MH+).

2-{3-[(5-Chloro-4-{5-[8-chloro-6-(trifluoromethyl)imidazo[1,2-a]pyridin-2-yl]-1,2,4-oxadiazol-3-yl}-2-fluorophenyl)oxy]pyrrolidin-1-yl}ethanol. MS (EI) for C₂₂H₁₇Cl₂F₄N₅O₃, found 546 (MH+).

{3-[(5-Chloro-4-{5-[8-chloro-6-(trifluoromethyl)imidazo[1,2-a]pyridin-2-yl]-1,2,4-oxadiazol-3-yl}-2-fluorophenyl)oxy]pyrrolidin-1-yl}acetic acid. MS (EI) for C₂₂H₁₅Cl₂F₄N₅O₄, found 560 (MH+).

2-[(2,5-Dichloro-4-{5-[8-chloro-6-(trifluoromethyl)imidazo[1,2-a]pyridin-2-yl]-1,2,4-oxadiazol-3-yl}phenyl)oxy]propan-1-ol. MS (EI) for C₁₉H₁₂Cl₃F₃N₄O₃, found 507 (MH+).

2-{3-[(3-Chloro-4-{5-[8-chloro-6-(trifluoromethyl)imidazo[1,2-a]pyridin-2-yl]-1,2,4-oxadiazol-3-yl}phenyl)oxy]pyrrolidin-1-yl}ethanol. MS (EI) for C₂₂H₁₈Cl₂F₃N₅O₃, found 528 (MH+).

{3-[(3-Chloro-4-{5-[8-chloro-6-(trifluoromethyl)imidazo[1,2-a]pyridin-2-yl]-1,2,4-oxadiazol-3-yl}phenyl)oxy]pyrrolidin-1-yl}acetic acid. MS (EI) for C₂₂H₁₆Cl₂F₃N₅O₄, found 542 (MH+).

{(2S,4S)-4-[(3-Chloro-4-{5-[8-chloro-6-(trifluoromethyl)imidazo[1,2-a]pyridin-2-yl]-1,2,4-oxadiazol-3-yl}phenyl)oxy]pyrrolidin-2-yl}methanol. MS (EI) for C₂₁H₁₆Cl₂F₃N₅O₃, found 514 (MH+).

{(2R,4S)-4-[(3-Chloro-4-{5-[8-chloro-6-(trifluoromethyl)imidazo[1,2-a]pyridin-2-yl]-1,2,4-oxadiazol-3-yl}phenyl)oxy]pyrrolidin-2-yl}methanol. MS (EI) for C₂₁H₁₆Cl₂F₃N₅ O₃, found 514 (MH+).

3-[(3-Chloro-4-{5-[8-chloro-6-(trifluoromethyl)imidazo[1,2-a]pyridin-2-yl]-1,2,4-oxadiazol-3-yl}phenyl)oxy]propanoic acid. MS (EI) for C₁₉th₁Cl₂F₃N₄O₄, found 487 (MH+).

3-[(3-Chloro-4-{5-[8-chloro-6-(trifluoromethyl)imidazo[1,2-a]pyridin-2-yl]-1,2,4-oxadiazol-3-yl}phenyl)oxy]propan-1-ol. MS (EI) for C ₁₉H₁₃Cl₂F₃N₄O₃, found 473 (MH+).

1-[(3-Chloro-4-{5-[8-chloro-6-(trifluoromethyl)imidazo[1,2-a]pyridin-2-yl]-1,2,4-oxadiazol-3-yl}phenyl)oxy]propan-2-amine. MS (EI) for C ₁₉H₁₄Cl₂F₃N₅ O₂, found 472 (MH+).

2-[(3-Chloro-4-{5-[8-chloro-6-(trifluoromethyl)imidazo[1,2-a]pyridin-2-yl]-1,2,4-oxadiazol-3-yl}phenyl)oxy]ethanamine. MS (EI) for C₁₈H₁₂Cl₂F₃N₅ O₂, found 459 (MH+).

2-[(3-Chloro-4-{5-[8-chloro-6-(trifluoromethyl)imidazo[1,2-a]pyridin-2-yl]-1,2,4-oxadiazol-3-yl}phenyl)oxy]propan-1-amine. MS (EI) for C ₁₉H₁₄Cl₂F₃N₅ O₂, found 472 (MH+).

2-[(2-Chloro-4-{5-[8-chloro-6-(trifluoromethyl)imidazo[1,2-a]pyridin-2-yl]-1,2,4-oxadiazol-3-yl}-6-fluorophenyl)oxy]-2-methylpropan-1-ol. MS (EI) for C₂₀H₁₄Cl₂F₄N₄O₃, found 507 (MH+).

2-[(3-Chloro-4-{5-[8-chloro-6-(trifluoromethyl)imidazo[1,2-a]pyridin-2-yl]-1,2,4-oxadiazol-3-yl}phenyl)oxy]propan-1-ol. ¹H-NMR (400 MHz, DMSO-d₆) δ 9.32 (s, 1H), 9.06 (s, 1H), 8.06 (s, 1H), 7.98-7.96 (d, 1H), 7.29 (s, 1H), 7.14-7.16 (d, 1H), 4.95-4.98(t, 1H), 4.61-4.66 (m, 1H), 3.52-3.57 (q, 2H), 1.26-1.29 (d, 3H); MS (EI) for C₁₉H₁₃Cl₂F₃N₄O₃, found 473 (MH+).

2-[(2,6-Dichloro-4-{5-[8-chloro-6-(trifluoromethyl)imidazo[1,2-a]pyridin-2-yl]-1,2,4-oxadiazol-3-yl}phenyl)oxy]ethyl acetate. ¹H-NMR (400 MHz, CDCl₃): δ 8.57 (s, 1H), 8.54 (s, 1H), 8.21 (s, 2H) 7.589 (s, 1H), 4.51-4.48 (t, 2H), 4.37-4.34 (t, 2H), 2.12 (s, 3H); MS (EI) for C₂₀H₁₂Cl₃F₃N₄O₄, found 534.9 (MH+).

1-[(4-{5-[8-Bromo-6-(trifluoromethyl)imidazo[1,2-a]pyridin-2-yl]-1,2,4-oxadiazol-3-yl}-2,5-dichlorophenyl)oxy]-3-fluoropropan-2-ol. MS (EI) for C₁₉H₁₁BrCl₂F₄N₄O₃, found 570.8 (MH+).

2-[(2,6-Dichloro-4-{5-[8-chloro-6-(trifluoromethyl)imidazo[1,2-a]pyridin-2-yl]-1,2,4-oxadiazol-3-yl}phenyl)oxy]ethanol. ¹H-NMR (400 MHz, DMSO-d₆) δ 9.341 (s, 1H), 9.06 (s, 1H), 8.13 (s, 2H) 8.08 (s, 1H), 4.97-4.94 (t, 1H), 4.15-4.13 (t, 2H), 3.82-3.78 (q, 2H); MS (EI) for C₁₈H₁₀Cl₃F₃N₄O₃, found 495 (MH+).

1-[(2,5-Dichloro-4-{5-[8-chloro-6-(trifluoromethyl)imidazo[1,2-a]pyridin-2-yl]-1,2,4-oxadiazol-3-yl}phenyl)oxy]propan-2-one. ¹H-NMR (400 MHz, DMSO-d₆) δ 9.33 (m, 1H), 9.07 (s, 1H), 8.12 (s, 1H), 8.07 (d, 1H), 7.46 (s, 1H), 5.19 (s, 2H), 2.21 (s, 3H); MS (EI) for C₁₉H₁₀Cl₃F₃N₄O₃, found 505.0 (MH+).

8-Chloro-2-(3-{2,5-dichloro-4-[(phenylmethyl)oxy]phenyl}-1,2,4-oxadiazol-5-yl)-6-(trifluoromethyl)imidazo[1,2-a]pyridine. ¹H-NMR (400 MHz, DMSO-d₆) δ 9.34 (s, 1H), 9.07 (s, 1H), 8.13 (s, 1H) 8.07 (s, 1H), 7.66 (s, 1H), 7.53-7.51 (d, 2H), 7.47-7.44 (t, 2H), 7.41-7.39 (m, 1H), 5.39 (s, 2H); MS (EI) for C₂₃H₁₂Cl₃F₃N₄O₂, found 538 (MH−).

8-Chloro-2-{3-[2,5-dichloro-4-({[4-(methyloxy)phenyl]methyl}oxy)phenyl]-1,2,4-oxadiazol-5-yl}-6-(trifluoromethyl)imidazo[1,2-a]pyridine. ¹H-NMR (400 MHz, DMSO-d₆) δ 9.34 (s, 1H), 9.07 (s, 1H), 8.12 (s, 1H) 8.07 (s, 1H), 7.66 (s, 1H), 7.45-7.44 (d, 2H), 7.01-6.99 (d, 2H), 5.29 (s, 2H), 3.78 (s, 3H); MS (EI) for C₂₄H₁₄Cl₃F₃N₄O₃, found 571 (MH+).

1-[(4-{5-[8-Bromo-6-(trifluoromethyl)imidazo[1,2-a]pyridin-2-yl]-1,2,4-oxadiazol-3-yl}-2,5-dichlorophenyl)oxy]propan-2-one. ¹H-NMR (400 MHz, DMSO-d₆) δ 9.35 (m, 1H), 9.08 (s, 1H), 8.17 (s, 1H), 8.11 (d, 1H), 7.46 (s, 1H), 5.19 (s, 2H), 2.21 (d, 3H); MS (EI) for C₁₉H₁₀BrCl₂F₃N₄O₃, found 550.9 (MH+).

2-[(2,5-Dichloro-4-{5-[8-chloro-6-(trifluoromethyl)imidazo[1,2-a]pyridin-2-yl]-1,2,4-oxadiazol-3-yl}phenyl)oxy]-2-methylpropan-1-ol. ¹H-NMR (400 MHz, DMSO-d₆) δ 9.32 (s, 1H), 9.08 (s, 1H), 8.12 (s, 1H), 8.08 (d, 1H), 7.71 (s, 1H), 5.22 (t, 1H), 3.53 (d, 2H), 1.33 (s, 6H); MS (EI) for C₂₀H₁₄Cl₃F₃N₄O₃, found 521 (MH+).

### Example 24

### 3-(5-Chloro-4-(5-(8-chloro-6-(trifluoromethyl)imidazo(1,2-a]pyridine-2-yl)-1,3,4-oxadiazol-2-yl)-2-fluorophenyl)propanoic acid

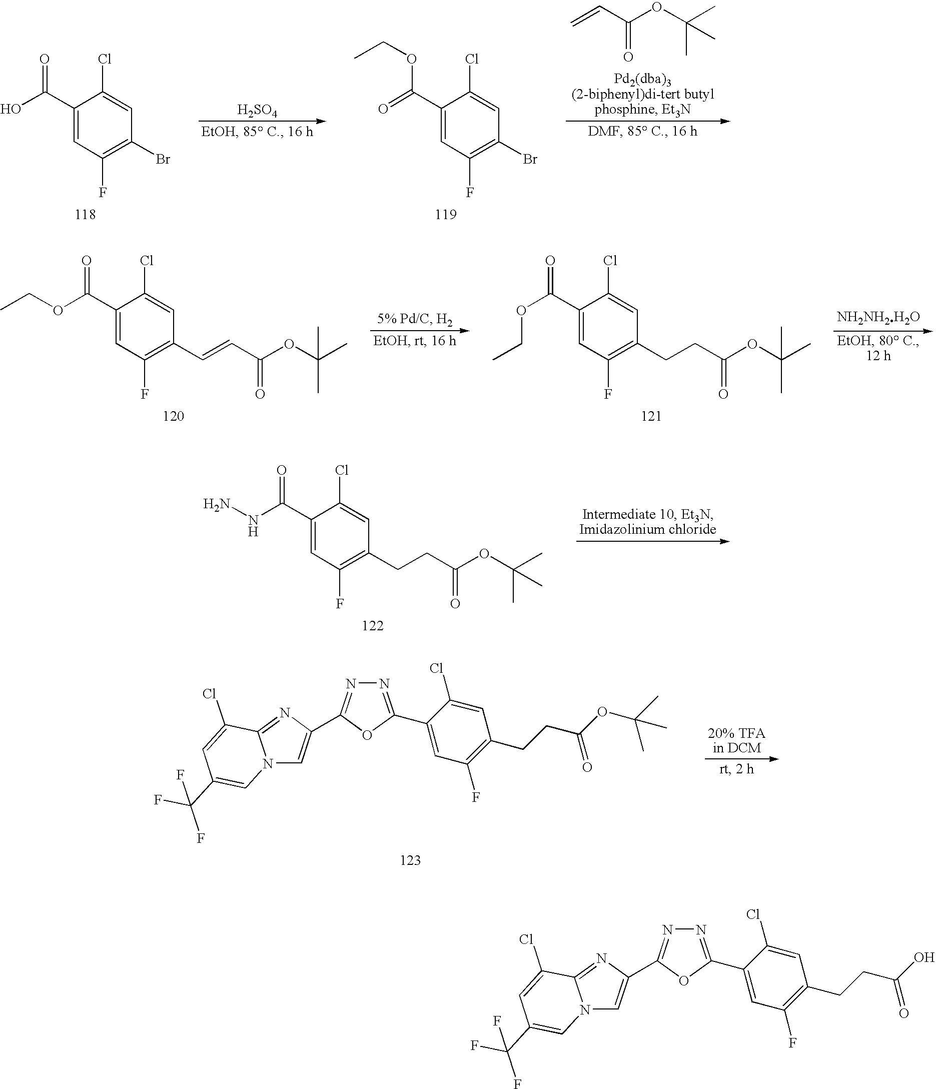

Ethyl 4-bromo-2-chloro-5-fluoro benzoate (119). To a stirred solution of 4-bromo-2-chloro-5-fluoro benzoic acid 118 (10.0 g, 40 mmol) in EtOH (50 mL) was added H₂SO₄ (5 mL) dropwise. After addition was complete, the reaction mixture was heated to 85° C. for 16 h. EtOH was removed under reduced pressure, the reaction mixture neutralized with saturated sodium bicarbonate solution and extracted with EtOAc. The combined organic layers were washed with saturated NaCl, dried over Na₂SO₄ and concentrated to afford the title intermediate 119 as an oil (9.0 g, 81% yield).

Ethyl 4-(3-tert-butoxy-3-oxoprop-1-enyl)-2-chloro-5-methylbenzoate (120). To a stirred solution of ethyl (4-bromo-2-chloro-5-fluoro)-benzoate (119) (9.0 g, 32 mmol) in DMF (50 mL) was added tent-butyl acrylate (5.6 mL, 38 mmol) and the reaction was degassed using argon for 30 min. Pd₂(dba)₃ (0.665 g, 1 mmol) and (2-biphenyl)-di-tent-butylphosphine (0.938 g, 3 mmol) were added followed by addition of triethylamine (5.3 mL, 38 mmol) and the reaction was again degassed for 30 min. The reaction was stirred at 85° C. for 16 h. The reaction mixture was filtered and the filtrate was concentrated. The residue was diluted with cold water (50 mL) and extracted with EtOAc. The combined organic layers were washed with saturated NaCl solution, dried over Na₂SO₄ and concentrated. The crude product was purified by column chromatography (100-200 mesh), using 2% EtOAc/hexane as eluent, to afford the title intermediate 120 (8.0 g, 76% yield).

Ethyl-4-(3-tent-butoxy-3-oxopropyl)-2-chloro-5-fluorobenzoate (121). To a stirred solution of intermediate 120 (8.0 g, 24 mmol) in EtOH (25 mL) was added 5% Pd/C (0.8 g) and the reaction was stirred under hydrogen atmosphere for 16 h at room temperature. After completion, the reaction was filtered through Celite and the filtrate concentrated in vacuo to afford the title intermediate 121 as an oil (8.0 g).

tert-Butyl-3-(5-chloro-2-fluoro-4-(hydrazine carbonyl)propanoate (122). To a stirred solution of intermediate 121 (9.0 g, 27 mmol) in EtOH (25 mL) was added hydrazine hydrate (1.6 mL, 32 mmol) and the resulting mixture was stirred at 80° C. for 12 h. After completion, solvent was evaporated under reduced pressure and the residue was diluted with EtOAc (100 mL). The organic phase was washed with water, dried over Na₂SO₄ and concentrated to afford the title intermediate 122 as a solid (4.0 g, 46% yield).

tert-Butyl-3-(5-chloro-4-(5-(8-chloro-6-(trifluoromethyl)imidazo(1,2-a]pyridine-2-yl)-1,3,4-oxadiazol-2-yl)-2-fluorophenyl)propanoate (123). To a stirred solution of Intermediate 10 (3.34 g, 12.7 mmol) in DCM (40 mL) was added imidazolinium chloride (4.28 g, 40 mmol) followed by hydrazide 122 (4.0 g, 13 mmol). The reaction was cooled to 0° C. and triethylamine (7.04 mL, 50 mmol) was added dropwise over a period of 10 min. After addition was complete, the reaction was allowed to warm to room temperature and stirred for 16 h. After completion, the reaction mixture was diluted with DCM (100 mL) and washed with saturated sodium bicarbonate solution. The organic phase was further washed with saturated NaCl, dried over Na₂SO₄ and concentrated under reduced pressure. The crude compound was recrystallized from iPrOH to afford the title intermediate 123 as a white solid (1.6 g, 23% yield).

3-(5-Chloro-4-(5-(8-chloro-6-(trifluoromethyl)imidazo(1,2-a]pyridine-2-yl)-1,3,4-oxadiazol-2-yl)-2-fluorophenyl)propanoic acid. A solution of intermediate 123 (1.6 g, 2.9 mmol) in 20% TFA in DCM was stirred at room temperature for 2 h. After completion, solvent was evaporated in vacuo to obtain an oily compound which was azeotropically distilled over toluene (10 mL x 2). The resulting solid was washed with iPrOH and ether to afford the title compound as a white solid (1.4 g, 98% yield). ¹H NMR (400 MHz, CDCl₃) δ 12.40 (br s, 1H), 9.25 (s, 1H), 8.98 (s, 1H), 8.0 (s, 1H), 7.85 (d, 1H), 7.75 (d, 1H), 2.97 (t, 2H), 2.60 (t, 2H); MS (EI) for C₁₉H₁₀Cl₂F₄N₄O₃, found 489 (MH+).

The following compounds were prepared using the same or analogous synthetic techniques in Example 24 and substituting with appropriate reagents, which were commercially available or were prepared using procedures herein or procedures known to one of ordinary skill in the art.

8-Chloro-2-[5-(2,6-difluorophenyl)-1,3,4-oxadiazol-2-yl]-6-(trifluoromethyl)imidazo[1,2-a]pyridine. MS (EI) for C₁₆H₆ClF₅N₄O, found 401.0 (MH+).

8-Chloro-2-[5-(2-chlorophenyl)-1,3,4-oxadiazol-2-yl]-6-(trifluoromethyl)imidazo[1,2-a]pyridine. ¹H-NMR (400 MHz, CDCl₃) δ 8.57 (m, 2H), 8.13 (m, 1H) 7.63-7.43 (complex m, 4H).

3-[3-Chloro-4-({5-[8-chloro-6-(trifluoromethyl)imidazo[1,2-a]pyridin-2-yl]-1,3,4-oxadiazol-2-yl}amino)phenyl]propanoic acid. MS (EI) for C₁₉H₁₂Cl₂F₃N₅O₃, found 486 (MH+).

3-(3-Chloro-4-{5-[8-chloro-6-(trifluoromethyl)imidazo[1,2-a]pyridin-2-yl]-1,3,4-oxadiazol-2-yl}phenyl)propanoic acid. MS (EI) for C₁₉H₁₁Cl₂F₃N₄O₃, found 471 (MH+).

N-(3-Chloro-4-{5-[8-chloro-6-(trifluoromethyl)imidazo[1,2-a]pyridin-2-yl]-1,3,4-oxadiazol-2-yl}phenyl)methanesulfonamide. MS (EI) for C₁₇H₁₀Cl₂F₃N₅O₃S, found 492 (MH+).

3-(3-Chloro-4-{5-[8-chloro-6-(trifluoromethyl)imidazo[1,2-a]pyridin-2-yl]-1,3,4-oxadiazol-2-yl}phenyl)butanoic acid. MS (EI) for C₂₀H₁₃Cl₂F₃N₄O₃, found 485 (MH+).

### Example 25

### 3-(5-Chloro-4-(5-(8-chloro-6-(trifluoromethyl)imidazo[1,2-a]pyridin-2-yl)-1,3,4-thiadiazol-2-yl)-2-fluorophenyl)propanoic acid

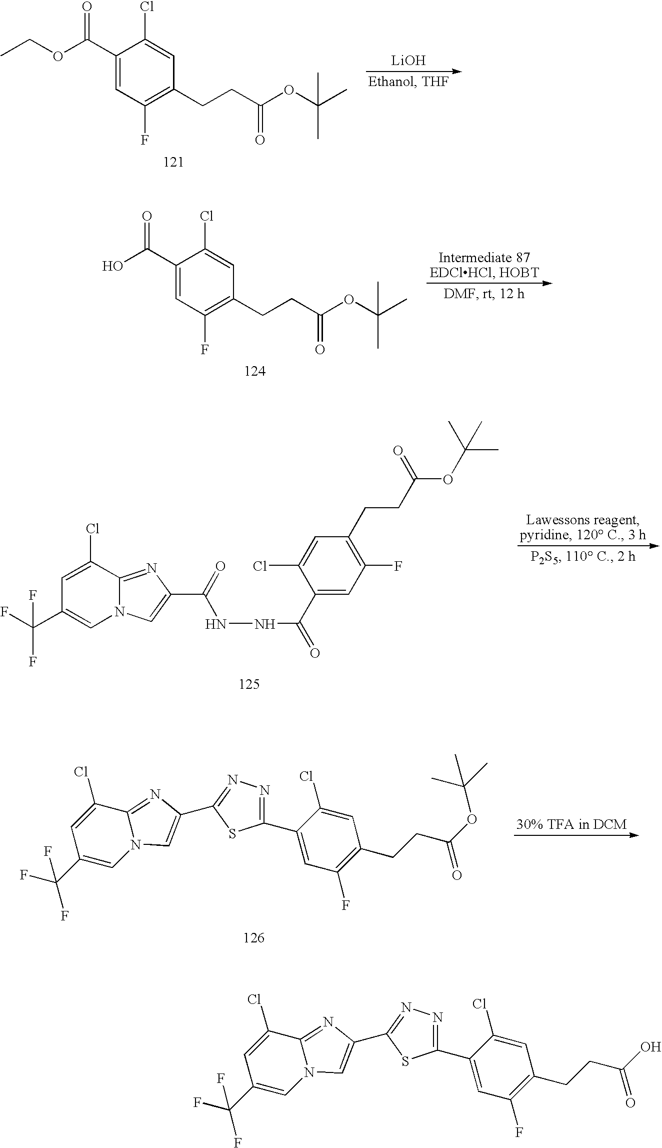

4-(3-tert-Butoxy-3-oxopropyl)-2-chloro-5-fluorobenzoic acid (124). To a stirred solution of intermediate 121 (3.0 g, 9.0 mmol) in EtOH/THF (1:1, 30 mL) was added 2 M aqueous lithium hydroxide solution (15 mL) at 0° C., followed by stirring at room temperature for 3 h. The reaction mixture was acidified with 10% citric acid solution and extracted with DCM. The organic layer was successively washed with water and saturated NaCl, dried over MgSO₄ and concentrated to give intermediate 124 (1.5 g, 55% yield) as a white solid.

tert-Butyl 3-(5-chloro-4-(2-(8-chloro-6-(trifluoromethyl)imidazo[1,2-a]pyridine-2-carbonyl)hydrazinecarbonyl)-2-fluorophenyl)propanoate. To a stirred solution of intermediate 87 (1.65 g, 5.92 mmol), prepared as described in Example 18, in dry DMF (25 mL) was added EDCI.HCl (1.2 g, 6.2 mmol) and HOBT (0.81 g, 6.0 mmol) and the mixture stirred at room temperature for 20 min, then intermediate 124 (1.5 g, 4.9 mmol) was added. The reaction mixture was stirred at room temperature for an additional 12 h. The reaction mixture was concentrated in vacuo and the residue dissolved in EtOAc and washed with saturated sodium bicarbonate solution (50 mL) and water, dried over Na₂SO₄ and concentrated. The intermediate was suspended in iPrOH and the resulting solids filtered and thoroughly dried to afford intermediate 125 (2.3 g, 83% yield) as an off-white solid.

tert-Butyl 3-(5-chloro-4-(5-(8-chloro-6-(trifluoromethyl)imidazo[1,2-a]pyridin-2-yl)-1,3,4-thiadiazol-2-yl)-2-fluorophenyl)propanoate. To a stirred solution of intermediate 125 (2.3 g, 4.1 mmol) in toluene (40 mL) was added pyridine (0.7 mL, 8.9 mmol) and Lawesson's reagent (2.15 g, 5.0 mmol) at room temperature. The reaction mixture was heated to reflux for 3 h. The reaction mixture was concentrated in vacuo and additional amounts of pyridine (35 mL, 450 mmol) and P₂S₅ (3.46 g, 15.6 mmol) were added at room temperature. The reaction mixture was returned to 110° C. for 2 h. The reaction mixture was quenched with ice water and the precipitated solid was filtered and washed successively with water and hexane and thoroughly dried. The obtained solid was recrystallized in chilled iPrOH to give intermediate 126 (1.0 g, 43% yield) as a white solid.

3-(5-Chloro-4-(5-(8-chloro-6-(trifluoromethyl)imidazo[1,2-a]pyridin-2-yl)-1,3,4-thiadiazol-2-yl)-2-fluorophenyl)propanoic acid. To a stirred solution of intermediate 126 (0.5 g, 9 mmol) in DCM (20 mL) was added TFA (7.5 mL) at 0° C., followed by stirring at room temperature overnight. The reaction mixture was concentrated in vacuo. The residue was cooled to 0° C. and suspended in iPrOH. The resulting solid was filtered and thoroughly dried to afford the title compound (0.2 g, 44% yield) as an off-white solid. ¹H NMR (400 MHz, DMSO-d₆) δ 9.4 (s, 1H), 8.9 (s, 1H), 8.1(d, 1H), 8.0 (s, 1H), 7.80 (d, 1H), 3.0 (m, 2H), 2.62 (m, 2H); MS (EI) for C₁₉H₁₀Cl₂F₄N₄O₂S, found 505 (MH+).

The following compounds were prepared using the same or analogous synthetic techniques in Example 25 and substituting with appropriate reagents, which were commercially available or were prepared using procedures herein or procedures known to one of ordinary skill in the art.

3-(2,6-Dichloro-4-{5-[8-chloro-6-(trifluoromethyl)imidazo[1,2-a]pyridin-2-yl]-1,3,4-thiadiazol-2-yl}phenyl)propanoic acid. MS (EI) for C₁₉H₁₀Cl₃F₃N₄O₂S, found 521 (MH+).

3-{5-Chloro-4-[5-(8-chloro-6-iodoimidazo[1,2-a]pyridin-2-yl)-1,3,4-thiadiazol-2-yl]-2-fluorophenyl}propanoic acid. ¹H-NMR (400 MHz, DMSO-d₆) δ 9.02 (s, 1H), 8.72 (s, 1H), 8.05 (d, 1H), 7.88 (s, 1H), 7.73 ds, 1H), 2.92 (t, 2H), 2.62 (t, 2H); MS (EI) for C₁₈H₁₀Cl₂FIN₄O₂S, found 563 (MH+).

3-(2,5-Dichloro-4-{5-[8-chloro-6-(trifluoromethyl)imidazo[1,2-a]pyridin-2-yl]-1,3,4-thiadiazol-2-yl}phenyl)-2-methylpropanoic acid. MS (EI) for C₂₀H₁₂Cl₃F₃N₄O₂S, found 535 (MH+).

3-(4-{5-[8-Bromo-6-(trifluoromethyl)imidazo[1,2-a]pyridin-2-yl]-1,3,4-thiadiazol-2-yl}-2,5-dichlorophenyl)propanoic acid. MS (EI) for C₁₉H₁₀BrCl₂F₃N₄O₂S, found 566.7 (MH+).

3-(3-Chloro-4-{5-[8-chloro-6-(trifluoromethyl)imidazo[1,2-a]pyridin-2-yl]-1,3,4-thiadiazol-2-yl}phenyl)propanoic acid. MS (EI) for C₁₉H₁₁Cl₂F₃N₄O₂S, found 487.0 (MH+).

3-(4-{5-[8-Bromo-6-(trifluoromethyl)imidazo[1,2-a]pyridin-2-yl]-1,3,4-thiadiazol-2-yl}-5-chloro-2-fluorophenyl)propanoic acid. MS (EI) for C₁₉H₁₀BrClF₄N₄O₂S, found 550.9 (MH+).

2-(2,5-Dichloro-4-{5-[8-chloro-6-(trifluoromethyl)imidazo[1,2-a]pyridin-2-yl]-1,3,4-thiadiazol-2-yl}phenyl)cyclopropanecarboxylic acid. MS (EI) for C₂₀H₁₀Cl₃F₃N₄O₂S, found 533 (MH+).

### Example 26

### 3-(2,5-Dichloro-4-(5-(8-chloro-6-(trifluoromethyl)imidazo[1,2-a]pyridin-2-yl)-1,2,4-oxadiazol-3-yl)phenoxy)-2-hydroxypropanoic acid

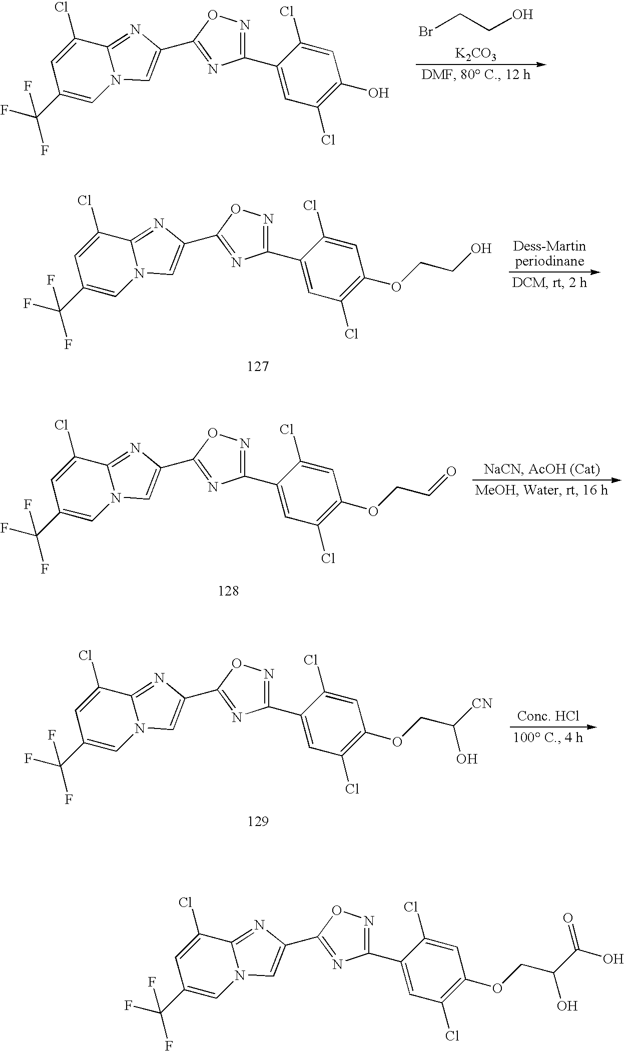

2-(2,5-Dichloro-4-(5-(8-chloro-6-(trifluoromethyl)imidazo[1,2-a]pyridin-2-yl)-1,2,4-oxadiazol-3-yl)phenoxy)ethanol. To a stirred solution of 2,5-dichloro-4-(5-(8-chloro-6-(trifluoromethyl)imidazo(1,2-a)pyridine-2-yl)-1,2,4-oxadiazole-3-yl)phenol (1.0 g, 2.4 mmol), prepared as described in Example 13, in DMF (10 mL) was added K₂CO₃ (1.66 g, 12.0 mmol) and 2-bromoethanol (1.5 g, 12 mmol). The reaction mixture was stirred at 80° C. for 12 h and then quenched with ice water and extracted with EtOAc. The organic layer was washed with water and saturated NaCl solution, dried over Na₂SO₄ and concentrated in vacuo to afford of intermediate 127 (0.70 g, 59% yield) as a yellowish-brown solid.

2-(2,5-Dichloro-4-(5-(8-chloro-6-(trifluoromethyl)imidazo[1,2-a]pyridin-2-yl)-1,2,4-oxadiazol-3-yl)phenoxy)acetaldehyde. To a stirred solution of intermediate 127 (0.70 g, 1.4 mmol) in DCM (10 mL) was added Dess-Martin periodinane (0.66 g, 1.5 mmol) and the reaction mixture was stirred at room temperature for 2 h. The reaction mixture was quenched with saturated sodium bicarbonate (10 mL) and Na₂S₂O₃ (10 mL) and extracted with DCM. The organic layer was washed with water and saturated NaCl solution, dried over Na₂SO₄ and concentrated in vacuo to afford of intermediate 128 (0.60 g, 86% yield) as a brown solid.

3-(2,5-Dichloro-4-(5-(8-chloro-6-(trifluoromethyl)imidazo[1,2-a]pyridin-2-yl)-1,2,4-oxadiazol-3-yl)phenoxy)-2-hydroxypropanenitrile. To a stirred solution of intermediate 128 (0.45 g, 0.91 mmol) in a mixture of MeOH (7 mL) and water (1 mL) was added acetic acid (catalytic amount). After stirring at room temperature for 1 h, NaCN (89 mg, 1.8 mmol) was added and the reaction mixture was stirred for 16 h at room temperature. The reaction mixture was concentrated in vacuo. Water was added to the residue and the mixture extracted with EtOAc. The organic layer was washed with water and saturated NaCl solution, dried over Na₂SO₄ and concentrated in vacuo to afford intermediate 129 (0.4 g, 84% yield) as a brown solid.

3-(2,5-Dichloro-4-(5-(8-chloro-6-(trifluoromethyl)imidazo[1,2-a]pyridin-2-yl)-1,2,4-oxadiazol-3-yl)phenoxy)-2-hydroxypropanoic acid. A stirred solution of intermediate 129 (0.35 g, 0.67 mmol) in concentrated HCl (3 mL) was heated to 100° C. for 4 h. The reaction mixture was concentrated in vacuo and purified by preparative HPLC to afford the title compound (35 mg, 9.7% yield) as a white solid. ¹H NMR (400 MHz, DMSO-d₆) δ 9.3 (s, 1H), 9.1 (s, 1H), 8.1 (d, 2H), 7.6 (s, 1H), 4.4 (m, 3H); MS (EI) for C₁₉H₁₀Cl₃F₃N₄O₅, found 537 (MH+).

Using the same or analogous synthetic techniques in Example 26 and substituting with appropriate reagents (prepared using procedures as described herein), 3-[(3-Chloro-4-{5-[8-chloro-6-(trifluoromethyl)imidazo[1,2-a]pyridin-2-yl]-1,2,4-oxadiazol-3-yl}phenyl)oxy]-2-hydroxypropanoic acid was prepared. MS (EI) for C₁₉H₁₁Cl₂F₃N₄O₅, found 503.0 (MH+).

### Example 27

### 1-(2,5-Dichloro-4-(5-(8-chloro-6-(trifluoromethyl)imidazo[1,2-a]pyridin-2-yl)-1,2,4-oxadiazol-3-yl)phenoxy)-2-methylpropan-2-ol

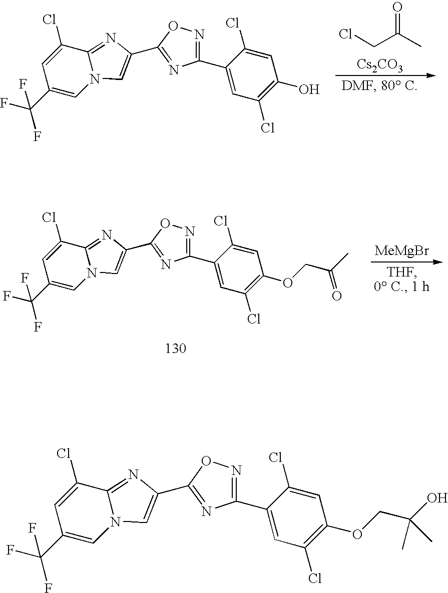

1-(2,5-Dichloro-4-(5-(8-chloro-6-(trifluoromethyl)imidazo[1,2-a]pyridin-2-yl)-1,2,4-oxadiazol-3-yl)phenoxy)propan-2-one. To a stirred solution of 2,5-dichloro-4-(5-(8-chloro-6-(trifluoromethyl)imidazo(1,2-a)pyridine-2-yl)-1,2,4-oxadiazole-3-yl)phenol (0.73 g, 1.6 mmol), prepared as described in Example 13, in DMF (10 mL) was added cesium carbonate (1.058 g, 3.24 mmol), and chloroacetone (0.4 mL, 4.9 mmol) at room temperature, followed by heating to 80° C. for 6 h. The reaction mixture was concentrated in vacuo and extracted with EtOAc. The organic layer was washed with water (2×), saturated NaCl solution, dried over Na₂SO₄ and concentrated in vacuo to afford intermediate 130 (0.73 g, 89% yield) as white solid.

1-(2,5-Dichloro-4-(5-(8-chloro-6-(trifluoromethyl)imidazo[1,2-a]pyridin-2-yl)-1,2,4-oxadiazol-3-yl)phenoxy)-2-methylpropan-2-ol. To a stirred solution of intermediate 130 (0.7 g, 1.4 mmol) in dry THF (8 mL) was added methyl magnesium bromide (1.0 mL, 2.8 mmol, 3 M) at 0° C. and the reaction mixture was stirred at 0° C. for 1 h. The reaction mixture was then quenched with saturated ammonium chloride, extracted with EtOAc and the organic layer was washed with water, saturated NaCl solution, dried over Na₂SO₄ and concentrated. The crude product was purified by column chromatography to afford the title compound (0.61 g, 83% yield) as white solid. ¹H NMR (400 MHz, DMSO-d₆) δ 9.39 (s, 1H), 9.15 (s, 1H), 8.17 (s, 1H), 8.16 (s, 1H), 7.49 (s, 1H), 3.90 (s, 2H), 1.22 (s, 6H); MS (EI) for C₂₀H₁₄Cl₃F₃N₄O₃, found 521 (MH+).

Using the same or analogous synthetic techniques in Example 27 and substituting with appropriate reagents (prepared using procedures as described herein), 1-[(4-{5-[8-Bromo-6-(trifluoromethyl)imidazo[1,2-a]pyridin-2-yl]-1,2,4-oxadiazol-3-yl}-2,5-dichlorophenyl)oxy]-2-methylpropan-2-ol was prepared. ¹H-NMR (400 MHz, DMSO-d₆) δ 9.33 (m, 1H), 9.06 (s, 1H), 8.15 (d, 1H), 8.08 (s, 1H), 7.51 (s, 1H), 4.73 (s, 1H), 3.96 (s, 2H), 1.23 (s, 6H); MS (EI) for C₂₀H₁₄BrCl₂F₃N₄O₃, found 567.0 (MH+).

### Example 28

### 3-(2,5-Dichloro-4-(3-(8-chloro-6-(trifluoromethyl)imidazo[1,2-a]pyridin-2-yl)-1,2,4-oxadiazol-5-yl)phenoxy)propane-1,2-diol

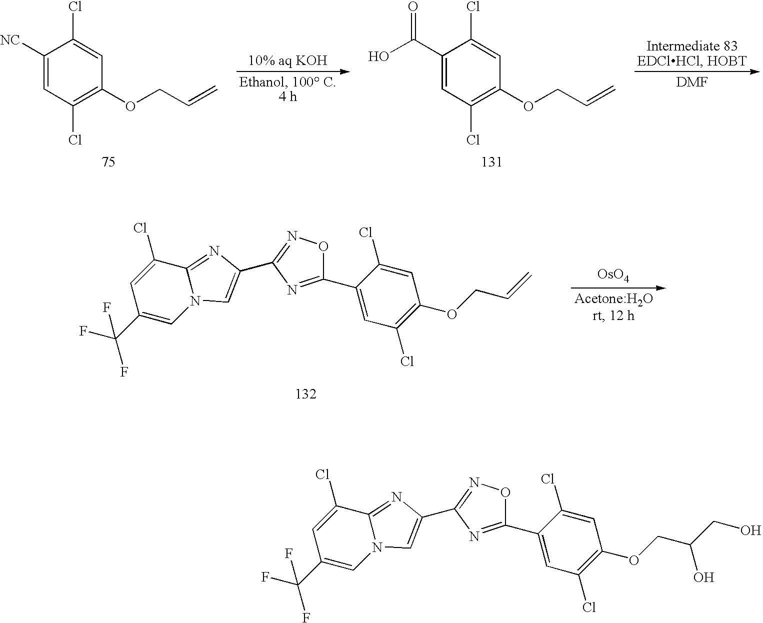

4-(Allyloxy)-2,5-dichlorobenzoic acid (131). To a stirred solution of intermediate 75 (1.5 g, 6.6 mmol) in EtOH (10 mL) was added aqueous 10% KOH solution (15 mL). The reaction mixture was stirred at room temperature for 20 min, followed by heating to 100° C. for 4 h. The reaction mixture was neutralized with 2 N HCl and extracted with EtOAc. The organic layer was washed with water, saturated NaCl solution, dried over Na₂SO₄ and concentrated to afford intermediate 131 (0.8 g, 49% yield) as a white solid.

5-(4-(Allyloxy)-2,5-dichlorophenyl)-3-(8-chloro-6-(trifluoromethyl)imidazo[1,2-a]pyridin-2-yl)-1,2,4-oxadiazole (132). To a stirred solution of hydroxyimidate 83 (0.85 g, 3.1 mmol) in DMF (5 mL) was added intermediate 131 (0.80 g, 3.2 mmol), EDCI.HCl (0.68 g, 3.5 mmol) and HOBT (0.52 g, 3.5 mmol). The mixture was stirred at room temperature for 1 h, followed by heating to 100° C. for 12 h. The reaction mixture was concentrated in vacuo. The resulting residue was dissolved in EtOAc and washed with sodium bicarbonate solution followed by saturated NaCl solution, dried over Na₂SO₄ and concentrated in vacuo. The residue was suspended in iPrOH, filtered and thoroughly dried to afford (0.4 g, 26% yield) of intermediate 132 as a white solid.

3-(2,5-Dichloro-4-(3-(8-chloro-6-(trifluoromethyl)imidazo[1,2-a]pyridin-2-yl)-1,2,4-oxadiazol-5-yl)phenoxy)propane-1,2-diol. To a stirred solution of intermediate 132 (300 mg, 0.61 mmol) in a mixture of acetone:water (9:1, 5 mL) was added OsO₄ (0.2 mL, 0.1 M solution in toluene) and NMO (2 mL) at room temperature. The resulting mixture was stirred at room temperature overnight. The reaction mixture was then quenched with saturated sodium sulfite solution and stirring was continued for an additional 45 min. The resulting solids were filtered, washed with water and ether to give the title compound (250 mg, 78% yield) as a white solid. ¹H NMR (400 MHz, DMSO-d₆) δ 9.3 (s, 1H), 8.9 (s, 1H), 8.3 (s, 1H), 8.0 (s, 1H), 7.6 (s, 1H), 5.1 (d, 1H, —OH), 4.8 (t, 1H, —OH), 4.2 (m, 2H), 3.9 (m, 1H), 3.5 (m, 2H); MS (EI) for C₁₉H₁₂Cl₃F₃N₄O₄, found 523 (MH+).

### Example 29

### 3-(2,5-Dichloro-4-(5-(8-chloro-6-(trifluoromethyl)imidazo[1,2-a]pyridin-2-yl)-1,2,4-oxadiazol-3-yl)phenyl)propane-1,2-diol

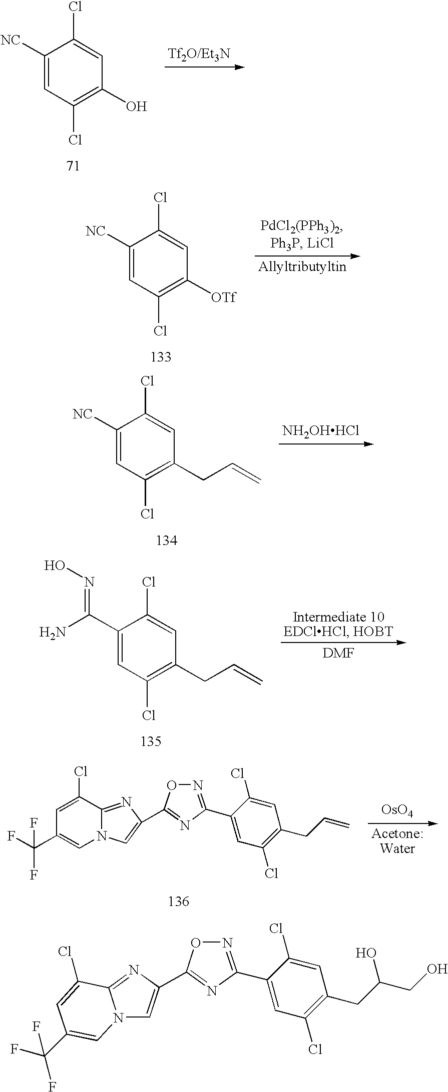

2,5-Dichloro-4-cyanophenyl trifluoromethanesulfonate (133). To a stirred solution of intermediate 71 (1.0 g, 5.3 mmol) in DCM (10 mL) was added triethylamine (1.03 mL, 7.47 mmol) at room temperature. It was cooled to −78° C., then triflic anhydride (1.08 mL, 6.41 mmol) was added dropwise to the reaction mixture and stirred for 1 h at −78° C. The reaction mixture was quenched with saturated sodium bicarbonate solution, extracted with EtOAc and concentrated in vacuo to give the crude intermediate 133 (1.7 g) which was used in subsequent reactions without further purification.

4-Allyl-2,5-dichlorobenzonitrile (134). A mixture of LiCl (768 mg, 18.1 mmol), PdCl₂(PPh₃)₂ (23 mg, 0.32 mmol) and PPh₃ (837 mg, 3.19 mmol) was degassed under high vacuum with an Argon purge. DMF (10 mL) was added with concomitant stirring followed by the addition of intermediate 133 (1.7 g, 5.3 mmol) and allyltributyltin (1.5 mL, 4.8 mmol). The resulting mixture was degassed. The reaction mixture was stirred at room temperature for 1 h and then heated to 60° C. until complete. After completion, the reaction mixture was cooled and diluted with EtOAc (30 mL) and washed with saturated NaCl (40 mL). The aqueous layer was further extracted with EtOAc (2×15 mL) and the combined organic layers were washed with saturated NaCl (2×40 mL) followed by water (2×40 mL), dried over Na₂SO₄ and concentrated. The residue was purified by column chromatography by using EtOAc/hexane to give intermediate 134 (0.97 g, 85%).

4-Allyl-2,5-dichloro-N′-hydroxybenzimidamide (135). To a stirred solution of hydroxylamine hydrochloride (3.2 g, 0.046 mol) in EtOH (10 mL) was added triethylamine (7.4 mL, 0.053 mol) dropwise at room temperature. After addition, the reaction mixture was stirred at room temperature for 1 h and then intermediate 134 (1.5 g, 0.007 mol) in EtOH (15 mL) was added. The reaction mixture was initially stirred at room temperature for 0.5 h followed by heating to 80° C. overnight. The reaction mixture was concentrated in vacuo to remove EtOH and extracted with EtOAc. The combined organic layers were washed with water and saturated NaCl, dried over Na₂SO₄ and concentrated to give intermediate 135 (1.3 g), which was used in subsequent reactions without further purification.

3-(4-Allyl-2,5-dichlorophenyl)-5-(8-chloro-6-(trifluoromethyl)imidazo[1,2-a]pyridin-2-yl)-1,2,4-oxadiazole (136). To a stirred solution of Intermediate 10 (1.4 g, 0.0053 mol) in dry DMF (10 mL) was added EDCI.HCl (1.5 g, 0.0078 mol), and HOBT (1 g, 0.007 mol) at room temperature and stirred 1 h. Intermediate 135 (1.3 g, 0.0053 mol) was added in dry DMF (5 mL) and further stirred at room temperature for 0.5 h, followed by heating to 100° C. for 14 h. The reaction mixture was concentrated in vacuo, the residue was partitioned between EtOAc and water. The phases were separated and the aqueous phase further extracted with EtOAc. The organic layer was washed with saturated NaCl, dried over Na₂SO₄ and concentrated. The crude product was purified by column chromatography using EtOAc/hexane to afford intermediate 136 (0.5 g, 20% yield) as a white solid.

3-(2,5-Dichloro-4-(5-(8-chloro-6-(trifluoromethyl)imidazo[1,2-a]pyridin-2-yl)-1,2,4-oxadiazol-3-yl)phenyl)propane-1,2-diol. To a stirred solution of intermediate 136 (0.5 g, 1.05 mmol) in a mixture of acetone:water (9:1, 5 mL) was added OsO₄ (0.2 mL, 0.1 M solution in toluene) and NMO (1 mL) at room temperature. After addition, it was stirred at room temperature overnight. The reaction mixture was quenched with saturated sodium sulfite solution and stirring was continued for an additional 45 min. The resulting solid was filtered, washed with water, then ether to give the title compound (0.38 g, 70% yield) as a white solid. ¹H NMR (400 MHz, DMSO-d₆) δ 9.29 (s, 1H), 9.22 (s, 1H), 8.20 (s, 2H), 7.80 (s, 1H), 5.62 (d, 1H), 4.62 (d, 1H), 4.80 (t, 1H), 3.80 (m, 2H), 2.45 (d, 2H); MS (EI) for C₁₉H₁₂Cl₃F₃N₄O₃, found 507 (MH+).

### Example 30

### 3-(2,5-Dichloro-4-(5-(8-chloro-6-(trifluoromethyl)imidazo[1,2-a]pyridin-2-yl)-1,2,4-oxadiazol-3-yl)phenyl)propan-1-ol

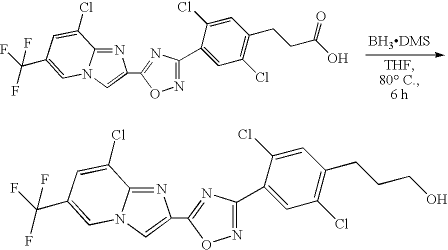

3-(2,5-Dichloro-4-(5-(8-chloro-6-(trifluoromethyl)imidazo[1,2-a]pyridin-2-yl)-1,2,4-oxadiazol-3-yl)phenyl)propan-1-ol. To a stirred solution of 3-(2,5-dichloro-4-(5-(8-chloro-6-(trifluoromethyl)-1H-imidazo[1,2-a]pyridin-2-yl)-1,2,4-oxadiazol-3-yl)phenyl)propanoic acid (0.3 g, 0.6 mmol), prepared as described in Example 20, in dry THF (5 mL) was added BH₃.DMS (0.4 mL, 4 mmol) dropwise at room temperature. After addition, it was heated to 80° C. for 6 h. The reaction mixture was quenched with MeOH at 0° C. followed by 2 N HCl, and extracted with EtOAc. The organic layer was dried over anhydrous Na₂SO₄ and concentrated in vacuo. The crude compound was purified by column chromatography using 50% EtOAc/hexane to afford the title compound (0.050 g, 17% yield) as an off-white solid. ¹H NMR (400 MHz, CDCl₃) δ 8.6 (s, 2H), 8.2 (s, 1H), 7.6 (s, 1H), 7.5 (s, 1H), 3.8 (m, 2H), 2.9 (m, 2H), 1.9 (m, 2H); MS (EI) for C₁₉H₁₂Cl₃F₃N₄O₂, found 491(MH+); HPLC (96.67%).

Using the same or analogous synthetic techniques in Example 30 and substituting with appropriate reagents, 4-(2,5-dichloro-4-{5-[8-chloro-6-(trifluoromethyl)imidazo[1,2-a]pyridin-2-yl]-1,2,4-oxadiazol-3-yl}phenyl)butan-2-ol was prepared. MS (EI) for C₂₀H₁₄Cl₃F₃N₄O₂, found 504.9 (MH+).

### Example 31

### 2-(2,5-Dichloro-4-(5-(8-chloro-6-(trifluoromethyl)imidazo[1,2-a]pyridin-2-yl)-1,2,4-oxadiazol-3-yl)phenoxy)ethanol

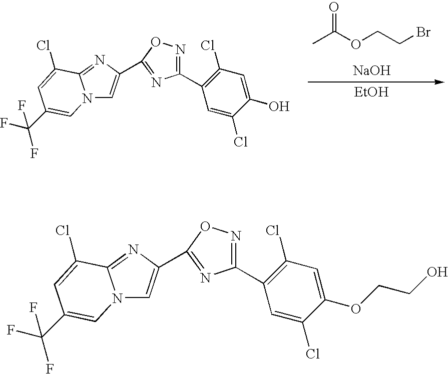

2-(2,5-Dichloro-4-(5-(8-chloro-6-(trifluoromethyl)imidazo[1,2-a]pyridin-2-yl)-1,2,4-oxadiazol-3-yl)phenoxy)ethanol. 2,5-dichloro-4-(5-(8-chloro-6-(trifluoromethyl)imidazo(1,2-a)pyridine-2-yl)-1,2,4-oxadiazole-3-yl)phenol (150 mg, 0.334 mmol), prepared as described in Example 13, and sodium hydroxide (20 mg, 0.5 mmol) were combined in EtOH (2 mL) and stirred at room temperature for 1 h, then 2-bromoethyl acetate (58 mg, 0.35 mmol) was added. The reaction mixture was heated to 80° C. for 16 h. Upon reaction completion, the mixture was diluted with EtOAc, washed with water, saturated NaCl, dried with Na₂SO₄, filtered and concentrated in vacuo. The residue was purified by column chromatography (30:70 EtOAc/hexanes) to yield 2-(2,5-dichloro-4-(5-(8-chloro-6-(trifluoromethyl)imidazo[1,2-a]pyridin-2-yl)-1,2,4-oxadiazol-3-yl)phenoxy)ethanol, the title compound (64 mg, 39% yield). ¹H-NMR (400 MHz, DMSO-d₆) δ 9.33 (s, 1H), 9.07 (s, 1H), 8.10 (s, 1H), 8.07 (s, 1H), 7.55 (s, 1H), 5.01 (t, 1H), 4.26 (t, 2H), 3.79 (m, 2H); MS (EI) for C₁₉H₁₂Cl₃F₃N₄O₃, found 493.0 (MH+).

### Example 32

### (S)-3-(2,5-Dichloro-4-(5-(8-chloro-6-(trifluoromethyl)imidazo[1,2-a]pyridin-2-yl)-1,2,4-oxadiazol-3-yl)phenoxy)propane-1,2-diol

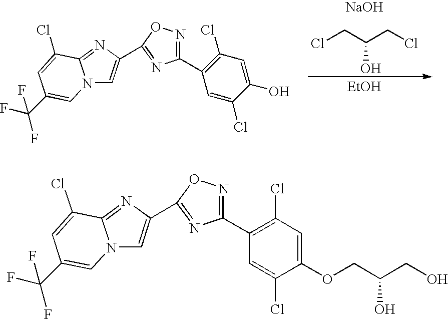

(S)-3-(2,5-Dichloro-4-(5-(8-chloro-6-(trifluoromethyl)imidazo[1,2-a]pyridin-2-yl)-1,2,4-oxadiazol-3-yl)phenoxy)propane-1,2-diol. 2,5-Dichloro-4-(5-(8-chloro-6-(trifluoromethyl)imidazo[1,2-a]pyridin-2-yl)-1,2,4-oxadiazol-3-yl)phenol (150 mg, 0.334 mmol), prepared as described in Example 13, and sodium hydroxide (20 mg, 0.5 mmol) were combined in EtOH (2 mL) and stirred at room temperature for 1 h, then (S)-3-chloropropane-1,2-diol (37 mg, 0.335 mmol) was added. The reaction mixture was heated to 80° C. for 16 h. Upon reaction completion, the mixture was diluted with EtOAc, washed with water, saturated NaCl, dried with Na₂SO₄, filtered and concentrated in vacuo. The residue was purified by preparative HPLC with NH₄OAc to give (S)-3-(2,5-dichloro-4-(5-(8-chloro-6-(trifluoromethyl)imidazo[1,2-a]pyridin-2-yl)-1,2,4-oxadiazol-3-yl)phenoxy)propane-1,2-diol (50 mg, 29% yield). ¹H-NMR (400 MHz, DMSO-d₆) δ 9.33 (s, 1H), 9.06 (s, 1H), 8.10 (s, 1H), 8.06 (s, 1H), 7.55 (s, 1H), 5.12 (d, 1H), 4.79 (m, 1H), 4.25 (m, 1H), 4.16 (m, 1H), 3.86 (m, 1H), 3.50 (m, 2H); MS (EI) for C₁₉H₁₂Cl₃F₃N₄O₄, found 523.0 (MH+).

The following compounds were prepared using the same or analogous synthetic techniques in Example 32 and substituting with appropriate reagents, which were commercially available or were prepared using procedures herein or procedures known to one of ordinary skill in the art.

(2R)-3-[(2,5-Dichloro-4-{5-[8-chloro-6-(trifluoromethyl)imidazo[1,2-a]pyridin-2-yl]-1,2,4-oxadiazol-3-yl}phenyl)oxy]propane-1,2-diol. ¹H-NMR (400 MHz, DMSO-d₆) δ 9.33 (s, 1H), 9.08 (s, 1H), 8.10 (s, 1H), 8.07 (s, 1H), 7.55 (s, 1H), 5.12 (d, 1H), 4.78 (t, 1H), 4.25 (m, 1H), 4.16 (m, 1H), 3.86 (m, 1H), 3.50 (t, 2H); MS (EI) for C₁₉H₁₂Cl₃F₃N₄O₄, found 523.0 (MH+).

(2S)-3-[(4-{5-[8-Bromo-6-(trifluoromethyl)imidazo[1,2-a]pyridin-2-yl]-1,2,4-oxadiazol-3-yl}-2,5-dichlorophenyl)oxy]propane-1,2-diol. ¹H-NMR (400 MHz, DMSO-d₆) δ 9.36 (m, 1H), 9.09 (s, 1H), 8.18 (d, 1H), 8.10 (s, 1H), 7.55 (s, 1H), 5.11 (d, 1H), 4.78 (t, 1H), 4.25 (dd, 1H), 4.16 (dd, 1H), 3.86 (m, 1H), 3.49 (t, 2H); MS (EI) for C₁₉H₁₂BrCl₂F₃N₄O₄, found 568.9 (M+H).

(2R)-3-[(4-{5-[8-Bromo-6-(trifluoromethyl)imidazo[1,2-a]pyridin-2-yl]-1,2,4-oxadiazol-3-yl}-2,5-dichlorophenyl)oxy]propane-1,2-diol. ¹H-NMR (400 MHz, DMSO-d₆) δ 9.36 (m, 1H), 9.09 (s, 1H), 8.18 (d, 1H), 8.10 (s, 1H), 7.55 (s, 1H), 5.11 (d, 1H), 4.78 (t, 1H), 4.25 (dd, 1H), 4.16 (dd, 1H), 3.86 (m, 1H), 3.49 (t, 2H); MS (EI) for C₁₉H₁₂BrCl₂F₃N₄O₄, found 568.9 (M+H).

### Example 33

### 1-(2,5-Dichloro-4-(5-(8-chloro-6-(trifluoromethyl)imidazo[1,2-a]pyridin-2-yl)-1,2,4-oxadiazol-3-yl)phenoxy)propan-2-ol

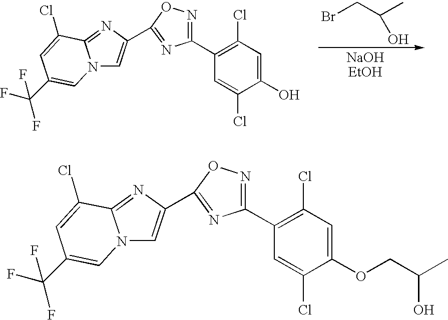

1-(2,5-Dichloro-4-(5-(8-chloro-6-(trifluoromethyl)imidazo[1,2-a]pyridin-2-yl)-1,2,4-oxadiazol-3-yl)phenoxy)propan-2-ol. 2,5-Dichloro-4-(5-(8-chloro-6-(trifluoromethyl)imidazo[1,2-a]pyridin-2-yl)-1,2,4-oxadiazol-3-yl)phenol (150 mg, 0.334 mmol), prepared as described in Example 13, and sodium hydroxide (20 mg, 0.5 mmol) were combined in EtOH (2 mL) and stirred at room temperature for 1 h, then 1-bromopropan-2-ol (80% pure, TCI America, 60 mg, 0.43 mmol) was added. The reaction mixture was heated to 80° C. for 16 h. Upon reaction completion, the mixture was diluted with EtOAc, washed with water, saturated NaCl, dried with Na₂SO₄, filtered and concentrated in vacuo. The residue was purified by column chromatography (30:70 EtOAc/hexanes) to yield 1-(2,5-dichloro-4-(5-(8-chloro-6-(trifluoromethyl)imidazo[1,2-a]pyridin-2-yl)-1,2,4-oxadiazol-3-yl)phenoxy)propan-2-ol (50 mg, 29% yield). ¹H-NMR (400 MHz, DMSO-d₆) δ 9.33 (s, 1H), 9.07 (s, 1H), 8.10 (s, 1H), 8.07 (s, 1H), 7.54 (s, 1H), 5.01 (d, 1H), 4.06 (m, 3H), 1.20 (m, 3H); MS (EI) for C₁₉H₁₂Cl₃F₃N₄O₃, found 507.0 (MH+).

The following compounds were prepared using the same or analogous synthetic techniques in Example 33 and substituting with appropriate reagents, which were commercially available or were prepared using procedures herein or procedures known to one of ordinary skill in the art.

3-[(4-{5-[8-Bromo-6-(trifluoromethyl)imidazo[1,2-a]pyridin-2-yl]-1,2,4-oxadiazol-3-yl}-2,5-dichlorophenyl)oxy]-1,1,1-trifluoropropan-2-ol. MS (EI) for C₁₉H₉BrCl₂F₆N₄O₃, found 606.7 (M+H).

1-[(2,6-Dichloro-4-{5-[8-chloro-6-(trifluoromethyl)imidazo[1,2-a]pyridin-2-yl]-1,2,4-oxadiazol-3-yl}phenyl)oxy]propan-2-ol. ¹H-NMR (400 MHz, DMSO-d₆) δ 9.34 (s, 1H), 9.06 (s, 1H), 8.12 (s, 1H), 8.06 (s, 1H), 4.97 (d, 1H), 4.02 (m, 2H), 3.87 (m, 1H), 1.23 (d, 3H); MS (EI) for C₁₉H₁₂Cl₃F₃N₄O₃, found 506.1 (M−H).

4-[(2,5-dichloro-4-{5-[8-chloro-6-(trifluoromethyl)imidazo[1,2-a]pyridin-2-yl]-1,2,4-oxadiazol-3-yl}phenyl)oxy]-3-oxobutanoic acid. ¹H-NMR (400 MHz, DMSO-d₆) δ 12.90 (br s, 1H), 9.35 (s, 1H), 9.05 (s, 1H), 8.10 (s, 1H), 8.0 (s, 1H), 7.50 (s, 1H), 5.25 (s, 2H) 3.65 (s, 2H).

### Example 34

### 3-(5-Chloro-2-fluoro-4-(5-(6-iodo-5-methylimidazo[1,2-a]pyridin-2-yl)-1,2,4-oxadiazol-3-yl)phenyl)propanoic acid

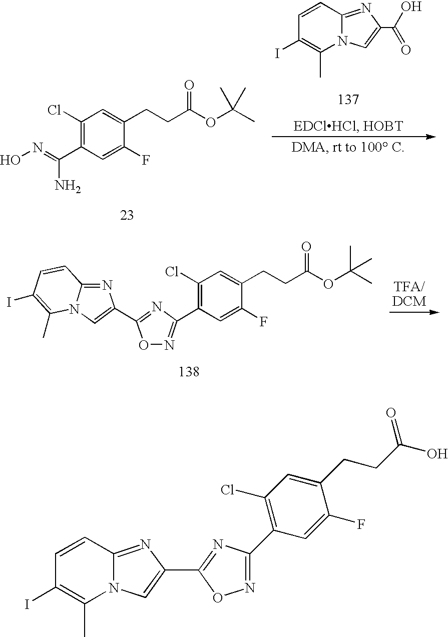

tert-Butyl 3-(5-chloro-2-fluoro-4-(5-(6-iodo-5-methylimidazo[1,2-a]pyridin-2-yl)-1,2,4-oxadiazol-3-yl)phenyl)propanoate (138). tert-Butyl 3-(5-chloro-2-fluoro-4-(N′-hydroxycarbamimidoyl)phenyl)propanoate 23 (100 mg, 0.316 mmol), prepared as described above in Example 2,6-iodo-5-methylimidazo[1,2-a]pyridine-2-carboxylic acid 137 (105 mg, 0.348 mmol), EDCI.HCl (66 mg, 0.35 mmol), and HOBT (47 mg, 0.35 mmol) were combined in N,N-dimethylacetamide (2 mL) and heated at 100° C. for 5 h. Upon reaction completion, the mixture was cooled to room temperature, diluted with EtOAc, washed with water, saturated NaCl, dried with Na₂SO₄, filtered and concentrated in vacuo. The residue was purified by column chromatography (15:85 EtOAc/hexanes) to yield tert-butyl 3-(5-chloro-2-fluoro-4-(5-(6-iodo-5-methylimidazo[1,2-a]pyridin-2-yl)-1,2,4-oxadiazol-3-yl)phenyl)propanoate 138 (110 mg, 60% yield).

3-(5-Chloro-2-fluoro-4-(5-(6-iodo-5-methylimidazo[1,2-a]pyridin-2-yl)-1,2,4-oxadiazol-3-yl)phenyl)propanoic acid. tert-Butyl 3-(5-chloro-2-fluoro-4-(5-(6-iodo-5-methylimidazo[1,2-a]pyridin-2-yl)-1,2,4-oxadiazol-3-yl)phenyl)propanoate 138 (109 mg, 0.187 mmol) was stirred in 30% TFA in dichloromethane (3.5 mL) for 1.5 h. Upon reaction completion, solvent was removed and the residue was triturated with ether to yield 3-(5-chloro-2-fluoro-4-(5-(6-iodo-5-methylimidazo[1,2-a]pyridin-2-yl)-1,2,4-oxadiazol-3-yl)phenyl)propanoic acid (87 mg, 88% yield). ¹H-NMR (400 MHz, DMSO-d₆) δ 12.33 (s, 1H), 8.94 (s, 1H), 8.74 (d, 1H), 7.74 (t, 1H), 7.47 (d, 1H), 2.93 (t, 2H), 2.90 (s, 3H), 2.64 (t, 2H); MS (EI) for C₁₉H₁₃ClFIN₄O₃, found 525.0 (MH+).

### Example 35

### 3-(5-Chloro-4-(5-(8-cyano-6-methylimidazo[1,2-a]pyridin-2-yl)-1,2,4-oxadiazol-3-yl)-2-fluorophenyl)propanoic acid

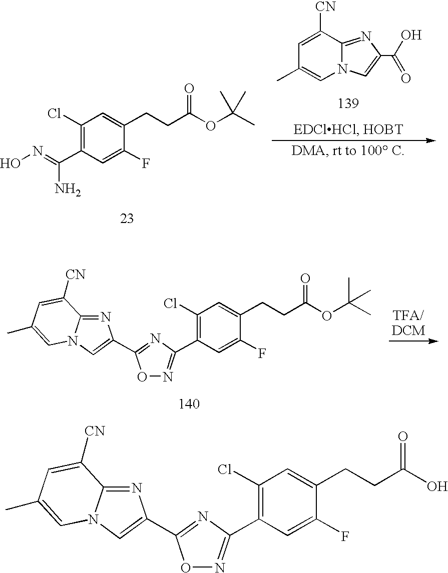

8-Cyano-6-methylimidazo[1,2-a]pyridine-2-carboxylic acid. Intermediate 139 was prepared using analogous methods to those used to make Intermediate 10, substituting 2-amino-5-methylnicotinonitrile for of 2-amino-3-chloro-5-trifluoromethyl pyridine in Intermediate 10.

tert-Butyl 3-(5-chloro-4-(5-(8-cyano-6-methylimidazo[1,2-a]pyridin-2-yl)-1,2,4-oxadiazol-3-yl)-2-fluorophenyl)propanoate (140). tert-Butyl 3-(5-chloro-2-fluoro-4-(N′- hydroxycarbamimidoyl)phenyl)propanoate 23 (100 mg, 0.316 mmol), prepared as described above in Example 2,8-cyano-6-methylimidazo[1,2-a]pyridine-2-carboxylic acid 139 (95 mg, 0.47 mmol), EDCI.HCl (91 mg, 0.47 mmol) and HOBT (64 mg, 0.47 mmol) were combined in N,N-dimethylacetamide (2 mL) and heated at 100° C. for 5 h. Upon reaction completion, the mixture was cooled to room temperature, diluted with EtOAc, washed with water, saturated NaCl, dried with Na₂SO₄, filtered and concentrated in vacuo. The residue was purified by column chromatography (30:70 EtOAc/hexanes) to yield tert-butyl 3-(5-chloro-4-(5-(8-cyano-6-methylimidazo[1,2-a]pyridin-2-yl)-1,2,4-oxadiazol-3-yl)-2-fluorophenyl)propanoate 140 (74 mg, 49% yield).

3-(5-Chloro-4-(5-(8-cyano-6-methylimidazo[1,2-a]pyridin-2-yl)-1,2,4-oxadiazol-3-yl)-2-fluorophenyl)propanoic acid. tert-Butyl 3-(5-chloro-4-(5-(8-cyano-6-methylimidazo[1,2-a]pyridin-2-yl)-1,2,4-oxadiazol-3-yl)-2-fluorophenyl)propanoate 140 (74 mg, 0.15 mmol) was stirred in 30% TFA in dichloromethane (2.8 mL) for 1.5 h. Upon reaction completion, solvent was removed and the residue was triturated with ether to yield 3-(5-chloro-4-(5-(8-cyano-6-methylimidazo[1,2-a]pyridin-2-yl)-1,2,4-oxadiazol-3-yl)-2-fluorophenyl)propanoic acid (52 mg, 79% yield). ¹H-NMR (400 MHz, DMSO-d₆) δ 12.33 (s, 1H), 9.02 (s, 1H), 8.77 (s, 1H), 8.15 (s, 1H), 7.84 (d, 1H), 7.73 (d, 1H), 2.94 (t, 2H), 2.65 (t, 2H), 2.37 (s, 3H); MS (EI) for C₂₀H₁₃ClFN₅O₃, found 426.1 (MH+).

### Example 36

### 3-(4-(5-(8-Chloro-6-(trifluoromethyl)imidazo[1,2-a]pyridin-2-yl)-1,2,4-oxadiazol-3-yl)-2,6-dimethylphenoxy)propane-1,2-diol

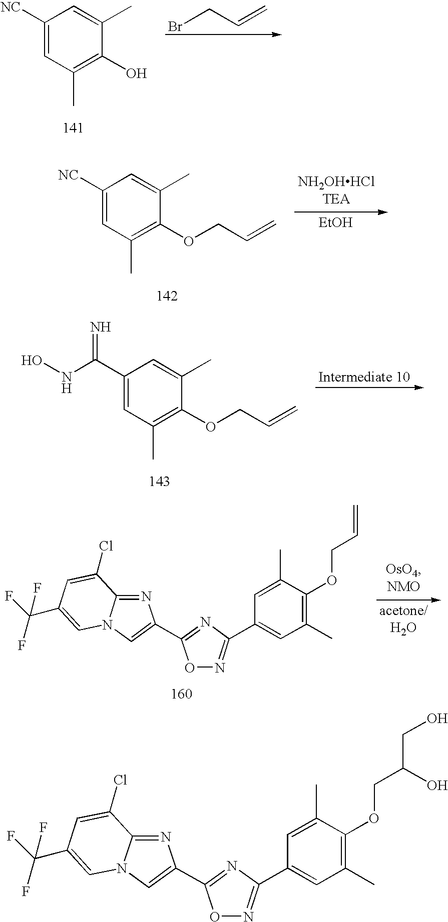

4-(Allyloxy)-3,5-dimethylbenzonitrile (142). To 4-hydroxy-3,5-dimethylbenzonitrile 141 (2.0 g, 14 mmol) in anhydrous acetone was added K₂CO₃ (4.51 g, 32.6 mmol) and allyl bromide (3.29 g, 27.2 mmol). The reaction mixture was stirred at room temperature for 72 h. The resulting mixture was filtered and the filtrate was concentrated in vacuo to dryness to yield 4-(allyloxy)-3,5-dimethylbenzonitrile 142 (2.58 g, 98.4% yield).

4-(Allyloxy)-N-hydroxy-3,5-dimethylbenzimidamide (143). To hydroxylamine hydrochloride (5.75 g, 82.7 mmol) in EtOH (45 mL) was added triethylamine (9.76 g, 96.5 mmol). The mixture was stirred at room temperature for 30 min, then 4-(allyloxy)-3,5-dimethylbenzonitrile 142 (2.58 g, 13.7 mmol) dissolved in EtOH (50 mL) was added. The resulting mixture was heated at 80° C. for 2 h. Upon reaction completion, solvent was removed in vacuo and the residue was diluted was EtOAc, washed with water, saturated NaCl, dried with Na₂SO₄, filtered, and concentrated in vacuo to yield 4-(allyloxy)-N-hydroxy-3,5-dimethylbenzimidamide 143 (2.96 g, 98.1% yield).

3-(4-(Allyloxy)-3,5-dimethylphenyl)-5-(8-chloro-6-(trifluoromethyl)imidazo[1,2-a]pyridin-2-yl)-1,2,4-oxadiazole. 4-(Allyloxy)-N-hydroxy-3,5-dimethylbenzimidamide 143 (500 mg, 2.27 mmol), 8-chloro-6-(trifluoromethyl)imidazo[1,2-a]pyridine-2-carboxylic acid 10 (661 mg, 2.50 mmol), EDCI.HCl (479 mg, 2.50 mmol), and HOBT (337 mg, 2.50 mmol) were combined in N,N-dimethylacetamide (15 mL) and heated at 100° C. for 5 h. Upon reaction completion, the mixture was cooled to room temperature, diluted with EtOAc, washed with water, saturated NaCl, dried with Na₂SO₄ and concentrated in vacuo. The residue was purified by column chromatography (10:90 EtOAc/hexanes) to yield 3-(4-(allyloxy)-3,5-dimethylphenyl)-5-(8-chloro-6-(trifluoromethyl)imidazo[1,2-a]pyridin-2-yl)-1,2,4-oxadiazole 160 (192 mg, 18.8% yield). ¹H-NMR (400 MHz, DMSO-d₆) δ 9.34 (t, 1H), 9.03 (s, 1H), 8.06 (d, 1H), 7.82 (s, 2H), 6.12 (m, 1H), 5.46 (m, 1H), 5.28 (m, 1H), 4.40 (m, 2H), 2.34 (s, 6H); MS (EI) for C₂₁H₁₆ClF₃N₄O₂, found 449.1 (MH+).

3-(4-(5-(8-Chloro-6-(trifluoromethyl)imidazo[1,2-a]pyridin-2-yl)-1,2,4-oxadiazol-3-yl)-2,6-dimethylphenoxy)propane-1,2-diol. To a mixture of 3-(4-(allyloxy)-3,5-dimethylphenyl)-5-(8-chloro-6-(trifluoromethyl)imidazo[1,2-a]pyridin-2-yl)-1,2,4-oxadiazole 160 (100 mg, 0.223 mmol) in acetone and water mixture (10:1) was added NMO (131 mg, 1.12 mmol) and OsO₄ (2.5 wt. % in t-butanol, 10 mg, 0.04 mmol). The reaction mixture was stirred at room temperature for 16 h. Upon reaction completion, the mixture was diluted with EtOAc, washed with water, saturated NaCl, dried with Na₂SO₄ and concentrated in vacuo. The crude product was purified by column chromatography (50:50 EtOAc/hexanes to 75:25 EtOAc/hexanes) to yield 3-(4-(5-(8-chloro-6-(trifluoromethyl)imidazo[1,2-a]pyridin-2-yl)-1,2,4-oxadiazol-3-yl)-2,6-dimethylphenoxy)propane-1,2-diol (78 mg, 72% yield). ¹H-NMR (400 MHz, DMSO-d₆) δ 9.34 (s, 1H), 9.03 (s, 1H), 8.06 (s, 1H), 7.80 (s, 2H), 5.01 (d, 1H), 4.68 (t, 1H), 3.84 (m, 2H), 3.75 (m, 1H), 2.30 (t, 2H), 2.35 (s, 6H); MS (EI) for C₂₁H₁₈ClF₃N₄O₄, found 483.0 (MH+).

### Example 37

### 3-(5-chloro-4-(5-(8-chloro-6-isopropoxyimidazo[1,2-a]pyridin-2-yl)-1,2,4-oxadiazol-3-yl)-2-fluorophenyl)propanoic acid

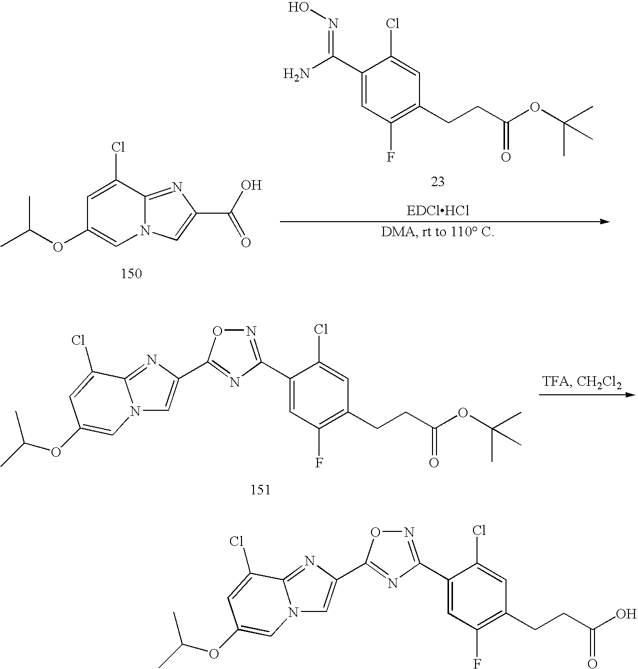

tert-Butyl 3-(5-chloro-4-(5-(8-chloro-6-isopropoxyimidazo[1,2-a]pyridin-2-yl)-1,2,4-oxadiazol-3-yl)-2-fluorophenyl)propanoate (151). To a solution of Intermediate 150 (340 mg, 1.33 mmol), prepared as described above, and tert-butyl 3-(5-chloro-2-fluoro-4-(N′-hydroxycarbamimidoyl)phenyl)propanoate 23 (420 mg, 1.33 mmol), prepared as described above in Example 2, in N,N-dimethylacetamide (5 mL) was added EDCI.HCl (379 mg, 1.98 mmol). The mixture was stirred for 1 h at room temperature. It was heated to 110° C. and the stirring was continued for 12 h. After cooling to room temperature, water was added, and this mixture was extracted with DCM. The extracts were combined, dried over Na₂SO₄ and concentrated. The crude intermediate 151 was further purified by column chromatography.

3-(5-Chloro-4-(5-(8-chloro-6-isopropoxyimidazo[1,2-a]pyridin-2-yl)-1,2,4-oxadiazol-3-yl)-2-fluorophenyl)propanoic acid. Intermediate 151 obtained above was treated with excess TFA in DCM (10 mL) for 2 h. The mixture was concentrated and slurried in ether. Filtration and washing with cold MeOH gave 3-(5-chloro-4-(5-(8-chloro-6-isopropoxyimidazo[1,2-a]pyridin-2-yl)-1,2,4-oxadiazol-3-yl)-2-fluorophenyl)propanoic acid (107 mg, 17% yield over two steps). ¹H-NMR (400 MHz, DMSO-d₆) M2.33 (br s, 1H), 8.85 (s, 1H), 8.41 (d, 1H), 7.83 (d, 1H), 7.72 (d, 1H), 7.56 (d, 1H), 4.56 (sep, 1H), 2.93 (t, 2H), 2.65 (t, 2H), 1.33 (d, 6H); MS (EI) for C₂₁H₁₇Cl₂FN₄O₄, found 479.1 (MH+).

### Example 38

### 3-(5-Chloro-4-(5-(8-chloro-6-iodoimidazo[1,2-a]pyridin-2-yl)-1,2,4-oxadiazol-3-yl)-2-fluorophenyl)propanoic acid

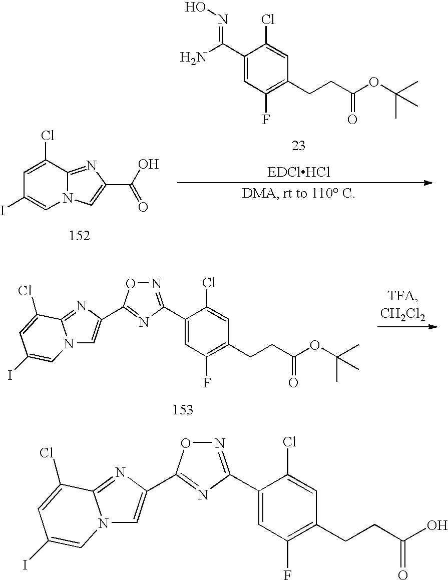

tert-Butyl 3-(5-chloro-4-(5-(8-chloro-6-iodoimidazo[1,2-a]pyridin-2-yl)-1,2,4-oxadiazol-3-yl)-2-fluorophenyl)propanoate (153). To a solution of 8-chloro-6-iodoimidazo[1,2-a]pyridine-2-carboxylic acid 152 (64 mg, 0.20 mmol), prepared as described above, and tert-butyl 3-(5-chloro-2-fluoro-4-(N’-hydroxycarbamimidoyl)phenyl)propanoate 23 (64 mg, 0.2 mmol), prepared as described above in Example 2, in N,N-dimethylacetamide (5 mL) was added EDCI.HCl (60 mg, 1.5 mmol). The mixture was stirred for 1 h at room temperature. It was then heated to 110° C. and the stirring was continued for 12 h. After cooling to room temperature, water was added and this mixture was extracted with DCM. The extracts were combined, dried over Na₂SO₄ and concentrated. Further purification by column chromatography gave tert-butyl 3-(5-chloro-4-(5-(8-chloro-6-iodoimidazo[1,2-a]pyridin-2-yl)-1,2,4-oxadiazol-3-yl)-2-fluorophenyl)propanoate 153 (32 mg, 26% yield).

3-(5-Chloro-4-(5-(8-chloro-6-iodoimidazo[1,2-a]pyridin-2-yl)-1,2,4-oxadiazol-3-yl)-2-fluorophenyl)propanoic acid. Intermediate 153 obtained above was treated with excess TFA in dichloromethane (10 mL) for 2 h. The mixture was concentrated and slurried in ether. Filtration and washing with cold MeOH gave 3-(5-chloro-4-(5-(8-chloro-6-iodoimidazo[1,2-a]pyridin-2-yl)-1,2,4-oxadiazol-3-yl)-2-fluorophenyl)propanoic acid (18 mg, 62% yield). ¹H-NMR (400 MHz, DMSO-d₆) M2.33 (br s, 1H), 9.01 (d, 1H), 8.89 (s, 1H), 7.93 (d, 1H), 7.83 (d, 1H), 7.72 (d, 1H), 2.93 (t, 2H), 2.65 (t, 2H); MS (EI) for C₁₈H₁₀Cl₂FIN₄O₃, found 547.0 (MH+).

### Example 39

### 3-(5-Chloro-4-(5-(8-chloro-6-(methylsulfonyl)imidazo[1,2-a]pyridin-2-yl)-1,2,4-oxadiazol-3-yl)-2-fluorophenyl)propanoic acid

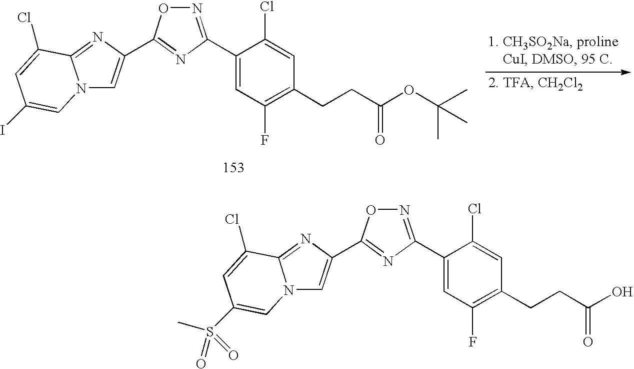

3-(5-Chloro-4-(5-(8-chloro-6-(methylsulfonyl)imidazo[1,2-a]pyridin-2-yl)-1,2,4-oxadiazol-3-yl)-2-fluorophenyl)propanoic acid. A mixture of tert-butyl 3-(5-chloro-4-(5-(8-chloro-6-iodoimidazo[1,2-a]pyridin-2-yl)-1,2,4-oxadiazol-3-yl)-2-fluorophenyl)propanoate 153 (150 mg, 0.25 mmol), sodium methanesulfinate (40 mg, 0.37 mmol), proline (6 mg, 0.05 mmol) and CuI (5.0 mg, 0.025 mmol) in DMSO (2 mL) in a sealed tube was heated to 95° C. for 12 h. The mixture was cooled to room temperature and purified by column chromatography. The tert-butyl ester was treated with excess TFA in CH₂Cl₂ to give 3-(5-chloro-4-(5-(8-chloro-6-(methylsulfonyl)imidazo[1,2-a]pyridin-2-yl)-1,2,4-oxadiazol-3-yl)-2-fluorophenyl)propanoic acid (1 mg, yield 1%). ¹H-NMR (400 MHz, DMSO-d₆) δ 12.25(br s, 1H), 9.38 (d, 1H), 9.19 (s, 1H), 8.12 (d, 1H), 7.84 (d, 1H), 7.73 (m, 2H), 3.41 (s, 3H), 2.93 (t, 2H), 2.65 (t, 2H); MS (EI) for C₁₉H₁₃Cl₂FN₄O₅S, found 499.0 (MH+).

### Example 40

### 3-(5-Chloro-4-(5-(8-chloro-6-isobutylimidazo[1,2-a]pyridin-2-yl)-1,2,4-oxadiazol-3-yl)-2-fluorophenyl)propanoic acid

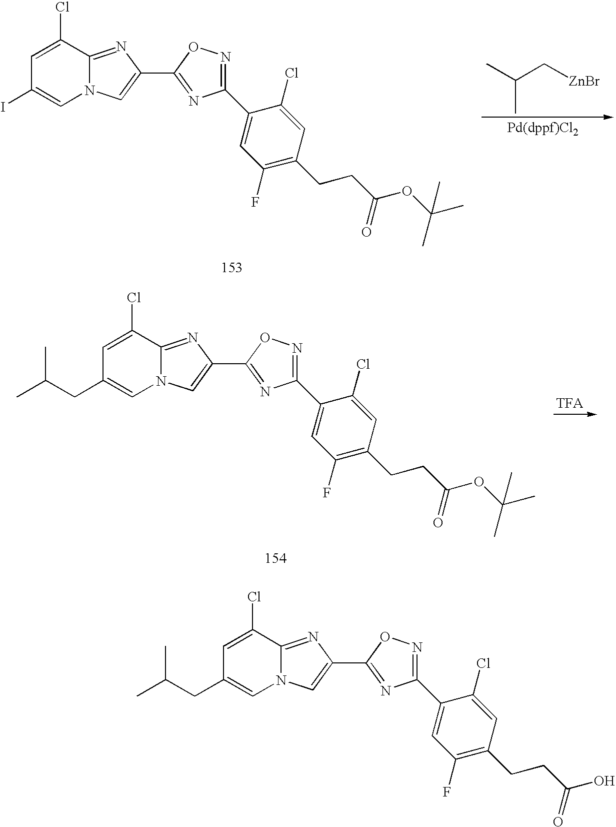

tert-Butyl 3-(5-chloro-4-(5-(8-chloro-6-isobutylimidazo[1,2-a]pyridin-2-yl)-1,2,4-oxadiazol-3-yl)-2-fluorophenyl)propanoate (154). Isobutylzinc bromide (0.5 M in THF, 1.8 mL) was added to a stirring solution of tert-butyl 3-(5-chloro-4-(5-(8-chloro-6-iodoimidazo[1,2-a]pyridin-2-yl)-1,2,4-oxadiazol-3-yl)-2-fluorophenyl)propanoate 153 (77 mg, 0.13 mmol), Pd(dppf)Cl₂ (10 mg, 0.014 mmol) and anhydrous THF (5 mL). The dark mixture was stirred at room temperature overnight. The reaction mixture was diluted with saturated ammonium chloride and extracted with EtOAc. The organic layer was dried over Na₂SO₄ and concentrated. Crude product was purified by column chromatography using 20/80 EtOAc/Hex to give tert-butyl 3-(5-chloro-4-(5-(8-chloro-6-isobutylimidazo[1,2-a]pyridin-2-yl)-1,2,4-oxadiazol-3-yl)-2-fluorophenyl)propanoate 154 (15 mg, 22% yield). ¹H-NMR (400 MHz, CDCl₃) δ 8.37 (s, 1H), 7.91 (s, 1H), 7.87-7.85 (d, 1H), 7.43-7.41 (d, 1H), 7.28 (dd, 1H), 3.00-2.96 (t, 2H), 2.61-2.58 (t, 2H), 2.50-4.48 (d, 2H), 1.95 (m, 1H), 0.99-0.97 (d, 6H); MS (EI) for C₂₆H₂₇Cl₂FN₄O₃, found 533 (MH+).

3-(5-Chloro-4-(5-(8-chloro-6-isobutylimidazo[1,2-a]pyridin-2-yl)-1,2,4-oxadiazol-3-yl)-2-fluorophenyl)propanoic acid. Intermediate 154 was treated with TFA as outlined in Example 38, Step 3 to give the title compound (11 mg, 82%). ¹H-NMR (400 MHz, DMSO-d₆) δ 8.94 (s, 1H), 8.44 (s, 1H), 7.83-7.80 (d, 1H), 7.71-7.70 (d, 1H), 7.60 (dd, 1H), 2.93-2.90 (t, 2H), 2.64-2.61 (t, 2H), 2.48-4.47 (d, 2H), 1.19 (m, 1H), 0.90-0.88 (d, 6H); MS (EI) for C₂₂H₁₉Cl₂FN₄O₃, found 477 (MH+).

### Example 41

### 8-Chloro-2-{3-[2,5-dichloro-4-(1[(4R)-2,2-dimethyl-1,3-dioxolan-4-yl]methyl}oxy)phenyl]-1,2,4-oxadiazol-5-yl}-6-(trifluoromethyl)imidazo[1,2-a]pyridine

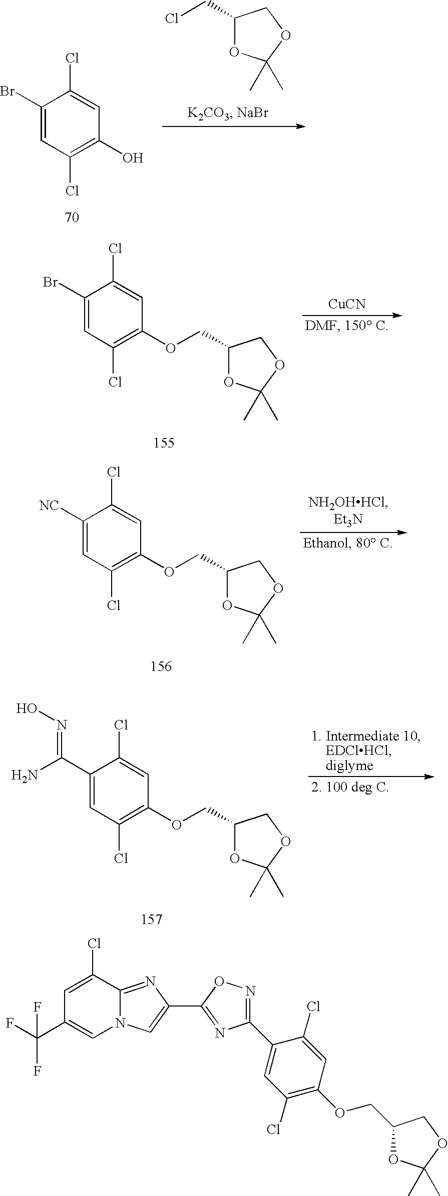

(R)-4-((4-Bromo-2,5-dichlorophenoxy)methyl)-2,2-dimethyl-1,3-dioxolane (155). To a solution of Intermediate 70 (30 g, 0.124 mol) in N,N-dimethylacetamide (496 mL) was added (S)-4-(chloromethyl)-2,2-dimethyl-1,3-dioxolane (33.6 g, 0.223 mol), potassium carbonate (42.8 g, 0.310 mol), and sodium bromide (15.3 g, 0.149 mol). The reaction was fitted with a reflux condenser and heated to 145° C. under an atmosphere of N₂ for 48 hours. Upon completion, the reaction was cooled to room temperature and filtered. The filtrate was slowly poured into a separatory funnel containing water. The aqueous layer was extracted with hexane, and the organic layer was separated and dried with MgSO₄. The organic layer was concentrated to yield a residue that was recrystallized in cold hexane, affording crystalline Intermediate 155 (30.1 g, 0.085 mol, 67.9% yield). ¹H-NMR (400 MHz, CDCl₃) δ 7.60 (s, 1H), 7.06 (s, 1H), 4.49 (m, 1H), 4.18 (m, 1H), 4.09 (m, 1H), 3.99 (dd, 2H), 1.47 (s, 3H), 1.41 (s, 3H); MS (EI) for C₁₂H₁₃BrCl₂O₃, found 357.0 (MH+).

(R)-2,5-Dichloro-4-((2,2-dimethyl-1,3-dioxolan-4-yl)methoxy)benzonitrile (156). (R)-4-((4-bromo-2,5-dichlorophenoxy)methyl)-2,2-dimethyl-1,3-dioxolane 155 (5.0 g, 14 mmol) and Copper (I) Cyanide (1.89 g, 21.1 mmol) in dimethylformamide (40 mL) were heated at 150° C. for 16 hours. Upon completion, the reaction mixture was concentrated, the residue was diluted with H₂O and the resulting solids filtered and rinsed with ethyl acetate multiple times. The filtrate was extracted with ethyl acetate, and then washed with brine. The organic layers were combined and dried over Na₂SO₄, filtered and concentrated in vacuo. The crude material was purified by flash chromatography on silica column with ethyl acetate/hexanes (15:85) to yield (R)-2,5-dichloro-4-((2,2-dimethyl-1,3-dioxolan-4-yl)methoxy)benzonitrile 156 (2.8 g, 66% yield). ¹H-NMR (400 MHz, DMSO-d₆) δ 8.20 (s, 1H), 7.63 (s, 1H), 4.44 (m, 1H), 4.33 (m, 2H), 4.10 (m, 1H), 3.80 (m, 1H), 1.35 (s, 3H), 1.30 (s, 3H).

(R,Z)-2,5-Dichloro-4-((2,2-dimethyl-1,3-dioxolan-4-yl)methoxy)-N′-hydroxybenzimidamide (157). To a solution of hydroxylamine hydrochloride (3.86 g, 55.5 mmol) in ethanol (45 mL) was added triethylamine (6.56 g, 64.8 mmol) and stirred at room temperature for 30 minutes. (R)-2,5-dichloro-4-((2,2-dimethyl-1,3-dioxolan-4-yl)methoxy)benzonitrile 156 (2.8 g, 9.3 mmol) dissolved in ethanol (55 mL) was added, and the reaction mixture was heated at 80° C. for 3.5 hours. The solvent was removed in vacuo and the residue was diluted with ethyl acetate, and washed with water and brine. The organic layers were combined and dried over Na₂SO₄, filtered, and concentrated in vacuo to yield (R,Z)-2,5-dichloro-4-((2,2-dimethyl-1,3-dioxolan-4-yl)methoxy)-N′-hydroxybenzimidamide 157 (3.08 g, 98.8% yield). ¹H-NMR (400 MHz, DMSO-d₆) δ 9.52 (s, 1H), 7.43 (s, 1H), 7.34 (s, 1H), 5.83 (s, 2H), 4.43 (m, 1H), 4.15 (m, 3H), 3.80 (dd, 1H), 1.36 (s, 3H), 1.31 (s, 3H); MS (EI) for C₁₃H₁₆C₁₂N₂O₄, found 335.1 (MH+).

8-Chloro-2-{3-[2,5-dichloro-4-({[(4R)-2,2-dimethyl-1,3-dioxolan-4-yl]methyl}oxy)phenyl]-1,2,4-oxadiazol-5yl}-6-(trifluoromethyl)imidazo[1,2-a]pyridine. To a mixture of (R,Z)-2,5-dichloro-4-((2,2-dimethyl-1,3-dioxolan-4-yl)methoxy)-N′-hydroxybenzimidamide 157 (3.08 g, 9.19 mmol) in diglyme (40 mL) was added Intermediate 10 (2.43 g, 9.18 mmol) and 1-(3-dimethylaminopropyl)-3-ethylcarbodiimide hydrochloride (1.76 g, 9.21 mmol). The reaction mixture was heated at 50° C. for 6 hours. When the coupling was complete by LC/MS, the reaction mixture was subsequently heated to 100° C. for 8 hours. The reaction was diluted with ethyl acetate, washed with water and brine. The organic layers were combined, dried over Na₂SO₄, filtered and concentrated in vacuo. The crude material was purified by flash chromatography (ethyl acetate/hexanes (20:80)), then triturated with hexanes to yield 8-chloro-2-{3-[2,5-dichloro-4-({[(4R)-2,2-dimethyl-1,3-dioxolan-4-yl]methyl}oxy)phenyl]-1,2,4-oxadiazol-5yl}-6-(trifluoromethyl)imidazo[1,2-a]pyridine as a white solid (2.03 g, 39.1% yield). ¹H-NMR (400 MHz, DMSO-d₆) δ 9.34 (m, 1H), 9.08 (s, 1H), 8.11 (s, 1H), 8.08 (s, 1H), 7.60 (s, 1H), 4.48 (m, 1H), 4.34 (m, 1H), 4.27 (m, 1H), 4.13 (dd, 1H), 3.84 (dd, 1H), 1.39 (s, 3H), 1.33 (s, 3H); MS (EI) for C₂₂H₁₆Cl₃F₃N₄O₄, found 563.0 (MH+).

### Example 42-A

### (2R)-3-[(2,5-Dichloro-4-{5-[8-chloro-6-(trifluoromethyl)imidazo[1,2-a]pyridin-2-yl]-1,2,4-oxadiazol-3-yl}phenyl)oxy]-2-hydroxypropyl dihydrogen phosphate and

### Example 42-B

### (S)-1-(2,5-dichloro-4-(5-(8-chloro-6-(trifluoromethyl)imidazo[1,2-a]pyridin-2-yl)-1,2,4-oxadiazol-3-yl)phenoxy)-3-hydroxypropan-2-yl dihydrogen phosphate

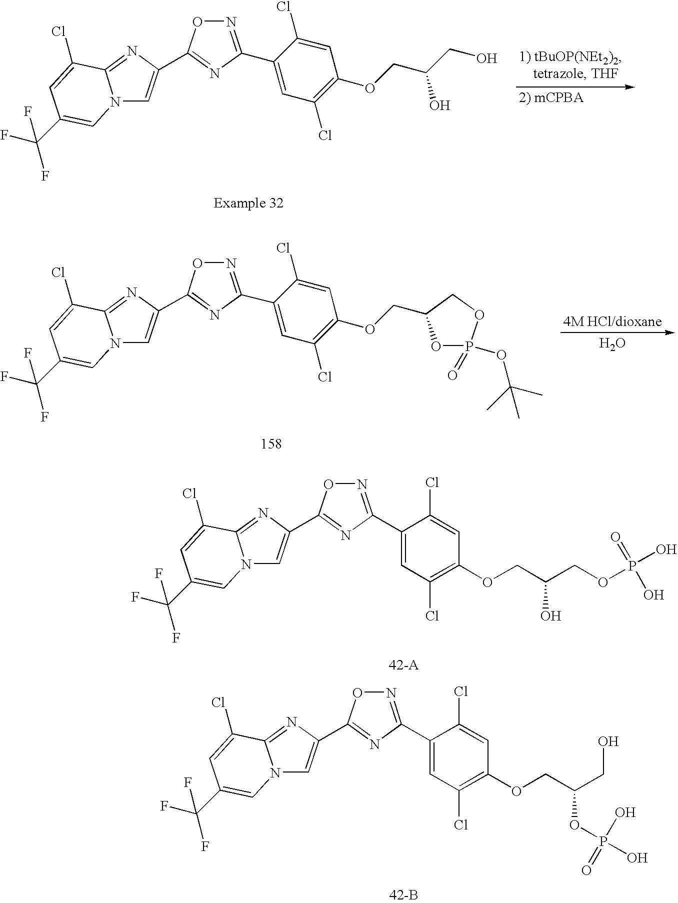

8-Chloro-2-(3-{4-[({(4S)-2-[(1,1-dimethylethyl)oxy]-2-oxido-1,3,2-dioxaphospholan-4-yl}methyl)oxy]-2,5-dimethylphenyl}-1,2,4-oxadiazol-5-yl)-6-(trifluoromethyl)imidazo[1,2-a]pyridine (158). To a stirred solution of (S)-3-(2,5-dichloro-4-(5-(8-chloro-6-(trifluoromethyl)imidazo[1,2-a]pyridin-2-yl)-1,2,4-oxadiazol-3-yl)phenoxy)propane-1,2-diol (200 mg, 0.38 mmol), prepared as described in Example 32, and tetrazole (80 mg, 1.14 mmol) in tetrahydrofuran (3 mL) at room temperature under nitrogen was added tert-butyl tetraisopropylphosphorodiamidite (174 mg, 0.57 mmol) slowly. The reaction mixture was stirred at room temperature overnight. LC/MS was used to monitor the formation of intermediate. Then the reaction mixture was cooled to −40° C. and m-chloroperbenzoic acid (100 mg, 0.57 mmol) was added in portions. The reaction mixture was allowed to warm to 0° C. and stirred for 2 hours before ethyl acetate was added. The solution was washed with aq. sodium sulfite, sat. NaHCO₃ and brine. The organic layers were combined, dried over Na₂SO₄, filtered and concentrated in vacuo to yield compound 158 (220 mg, 90% yield). The crude product was used for the next step without further characterization or delay.

(2R)-3-[(2,5-Dichloro-4-{5-[8-chloro-6-(trifluoromethyl)imidazo[1,2-a]pyridin-2-yl]-1,2,4-oxadiazol-3-yl}phenyl)oxy]-2-hydroxypropyl dihydrogen phosphate and (S)-1-(2,5-dichloro-4-(5-(8-chloro-6-(trifluoromethyl)imidazo[1,2-a]pyridin-2-yl)-1,2,4-oxadiazol-3-yl)phenoxy)-3-hydroxypropan-2-yl dihydrogen phosphate. Compound 158 (220 mg, 0.34 mmol) were dissolved in 4 N HCl/dioxane (2 mL) and 10 μL of water was added. The mixture was stirred at room temperature for 72 hours. The residue was triturated in ether and the solid was filtered and purified by prep HPLC with NH₄OAc to yield a 1:1 mixture of regio-isomers, 42-A and 42-B (18 mg, 8.8% combined yield of both isomers). ¹H-NMR (400 MHz, DMSO-d₆ with D₂O) δ 9.27 (m, 1H), 9.01 (d, 1H), 8.01 (d, 1H), 7.97 (m, 1H), 7.48 (d, 1H), 4.32 (m, 0.5H), 4.23 (m, 1H), 4.14 (m, 1H), 3.94 (m, 0.5H), 3.83 (m, 1H), 3.62 (m, 1H); MS (EI) for C₁₉H₁₃Cl₃F₃N₄O₇P, found 600.9 (M−H).

### Example 43

### (1R)-2-[(4-{5-[8-Bromo-6-(trifluoromethyl)imidazo[1,2-a]pyridin-2-yl]-1,2,4-oxadiazol-3-yl}-2,5-dichlorophenyl)oxy]-1-methylethyl dihydrogen phosphate

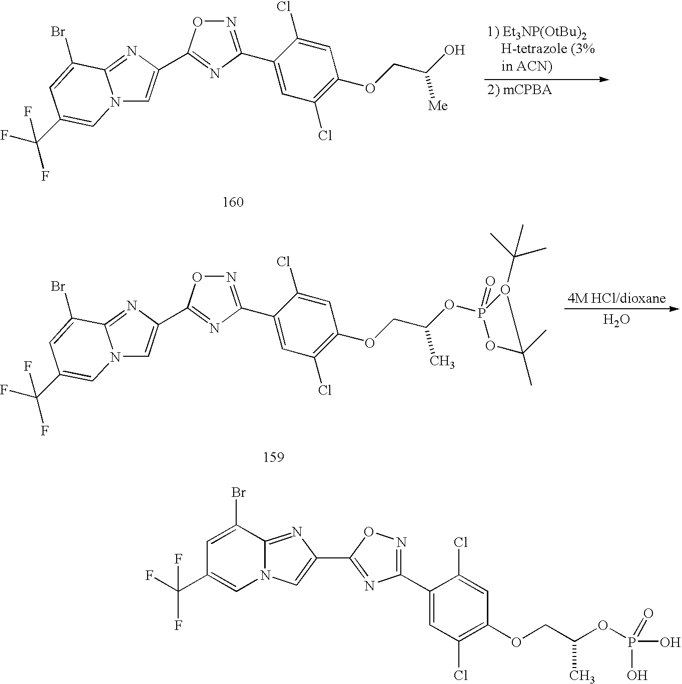

(R)-1-(4-(5-(8-bromo-6-(trifluoromethyl)imidazo[1,2-a]pyridin-2-yl)-1,2,4-oxadiazol-3-yl)-2,5-dichlorophenoxy)propan-2-ol (160). Intermediate 160 was made in a manner analogous to Example 44.

(1R)-2-[(4-{5-[8-Bromo-6-(trifluoromethyl)imidazo[1,2-a]pyridin-2-yl]-1,2,4-oxadiazol-3-yl}-2,5-dichlorophenyl)oxy]-1-methylethyl dihydrogen phosphate. Intermediate 160 (83.9 mg, 0.152 mmol) was added to a solution of tetrazole in acetonitrile (3 wt. %, 2.13 mL, 0.912 mmol). Di-tert-butyl diethyl phosphoramidite (151 mg, 0.608 mmol) was added and the solution was stirred for one hour. Upon consumption of Intermediate 160, the reaction was cooled to 0° C. and 3-chloroperbenzoic acid (104 mg, 0.608 mmol) was added portionwise. The reaction was warmed to room temperature. After 30 min, the reaction mixture was diluted with EtOAc and washed with NaHCO₃. The layers were separated and the aqueous layer was washed twice with EtOAc. All organic layers were combined, dried with MgSO₄, and concentrated to give crude Intermediate 159, which was then dissolved in 4 N HCl/dioxane solution (1 mL) and H₂O (15 μL). The reaction was stirred at room temperature for 1.5 hours, at which time the reaction was added to ether to yield a solid. The solvent was removed in vacuo, and the resulting solid was purified by preparative HPLC with NH₄OAc to yield the title compound (18.4 mg, 19% yield). ¹H-NMR (400 MHz, DMSO-d₆) δ 9.31 (s, 1H), 9.04 (s, 1H), 8.10 (s, 1H), 8.05 (s, 1H), 7.50 (s, 1H), 4.46 (m, 1H), 4.28 (m, 1H), 4.12 (m, 1H), 1.30 (d, 3H); MS (EI) for C₁₉H₁₃BrCl₂F₃N₄O₆P, found 630.9 (M−H).

The following compounds were prepared using the same or analogous synthetic techniques in Example 43 and substituting with appropriate reagents, which were commercially available or were prepared using procedures herein or procedures known to one of ordinary skill in the art.

(1S)-2-[(4-{5-[8-Bromo-6-(trifluoromethyl)imidazo[1,2-a]pyridin-2-yl]-1,2,4-oxadiazol-3yl}-2,5-dichlorophenyl)oxy]-1-methylethyl dihydrogen phosphate. ¹H-NMR (400 MHz, DMSO-d₆) δ 9.31 (s, 1H), 9.04 (s, 1H), 8.10 (s, 1H), 8.05 (s, 1H), 7.50 (s, 1H), 4.46 (m, 1H), 4.28 (m, 1H), 4.12 (m, 1H), 1.30 (d, 3H); MS (EI) for C₁₉H₁₃BrCl₂F₃N₄O₆P, found 630.9 (M−H).

(1R)-2-[(2,5-Dichloro-4-{5-[8-chloro-6-(trifluoromethyl)imidazo[1,2-a]pyridin-2-yl]-1,2,4-oxadiazol-3-yl}phenyl)oxy]-1-methylethyl dihydrogen phosphate. ¹H-NMR (400 MHz, DMSO-d₆) δ 9.28 (s, 1H), 9.03 (s, 1H), 8.04 (s, 1H), 7.97 (s, 1H), 7.48 (s, 1H), 4.45 (m, 1H), 4.28 (m, 1H), 4.09 (m, 1H), 1.29 (d, 3H); MS (EI) for C₁₉H₁₃Cl₃F₃N₄O₆P, found 585.0 (M−H).

(1S)-2-[(2,5-Dichloro-4-{5-[8-chloro-6-(trifluoromethyl)imidazo[1,2-a]pyridin-2-yl]-1,2,4-oxadiazol-3-yl}phenyl)oxy]-1-methylethyl dihydrogen phosphate. ¹H-NMR (400 MHz, DMSO-d₆) δ 9.28 (s, 1H), 9.03 (s, 1H), 8.04 (s, 1H), 7.97 (s, 1H), 7.48 (s, 1H), 4.45 (m, 1H), 4.28 (m, 1H), 4.09 (m, 1H), 1.29 (d, 3H); MS (EI) for C₁₉H₁₃Cl₃F₃N₄O₆P, found 585.0 (M−H).

### Example 44

### (2S)-1-[(2,5-Dichloro-4-{5-[8-chloro-6-(trifluoromethyl)imidazo[1,2-a]pyridin-2-yl]-1,2,4-oxadiazol-3-yl}phenyl)oxy]propan-2-ol

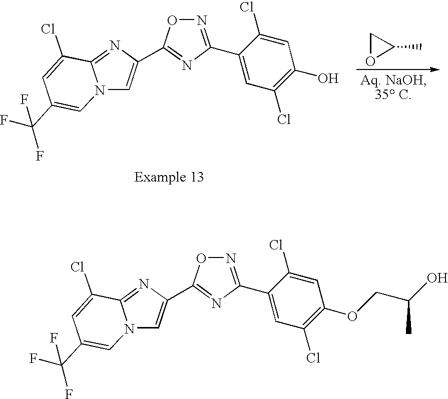

(2S)-1-[(2,5-Dichloro-4-{5-[8-chloro-6-(trifluoromethyl)imidazo[1,2-a]pyridin-2-yl]-1,2,4-oxadiazol-3-yl}phenyl)oxy]propan-2-ol. 2,5-Dichloro-4-(5-(8-chloro-6-(trifluoromethyl)imidazo(1,2-a)pyridine-2-yl)-1,2,4-oxadiazole-3-yl) phenol (1.0 g, 2.0 mmol), prepared as described in Example 13, was dissolved in THF (3 mL), followed by the addition of 1 M NaOH (2 mL, 2 mmol), and (S)-propylene oxide (580 mg, 10 mmol). The reaction mixture was stirred at 35° C. for 5 days. It was cooled to rt and diluted with EtOAc. The layers were separated and the organic phase was washed with H₂O and brine. The EtOAc solution was dried over Na₂SO₄ and concentrated to give the crude product which was purified by flash column chromatography to give the title compound (750 mg, 73% yield). ¹H-NMR (400 MHz, DMSO-d₆) δ 9.33 (m, 1H), 9.07 (s, 1H), 8.10 (s, 1H), 8.07 (d, 1H), 7.54 (s, 1H), 5.02 (d, 1H), 4.06 (m, 3H), 1.21 (d, 3H); MS (D) for C₁₉H₁₂Cl₃F₃N₄O₃, found 507.0 (MH+).

The following compounds were prepared using the same or analogous synthetic techniques in Example 44 and substituting with appropriate reagents, which were commercially available or were prepared using procedures herein or procedures known to one of ordinary skill in the art.

1-[(2,5-Dichloro-4-{5-[8-chloro-6-(trifluoromethyl)imidazo[1,2-a]pyridin-2-yl]-1,2,4-oxadiazol-3-yl}phenyl)oxy]-3-fluoropropan-2-ol. ¹H-NMR (400 MHz, DMSO-d₆) δ 9.33 (s, 1H), 9.08 (s, 1H), 8.11 (s, 1H), 8.07 (d, 1H), 7.58 (s, 1H), 5.63 (d, 1H), 4.59 (m, 1H), 4.48 (m, 1H), 4.25 (d, 2H), 4.13 (m, 1H); MS (D) for C₁₉th₁Cl₃F₄N₄O₃, found 525.0 (MH+).

(2R)-1-[(2,5-Dichloro-4-{5-[8-chloro-6-(trifluoromethyl)imidazo[1,2-a]pyridin-2-yl]-1,2,4-oxadiazol-3-yl}phenyl)oxy]propan-2-ol. ¹H-NMR (400 MHz, DMSO-d₆) δ 9.33 (m, 1H), 9.07 (s, 1H), 8.10 (s, 1H), 8.07 (d, 1H), 7.54 (s, 1H), 5.00 d, 1H), 4.06 (m, 3H), 1.21 (d, 3H); MS (D) for C₁₉H₁₂Cl₃F₃N₄O₃, found 507.0 (MH+).

1-[(2,5-Dichloro-4-{5-[8-chloro-6-(trifluoromethyl)imidazo[1,2-a]pyridin-2-yl]-1,2,4-oxadiazol-3-yl}phenyl)oxy]-3-(methyloxy)propan-2-ol. ¹H-NMR (400 MHz, DMSO-d₆) δ 9.33 (m, 1H), 9.07 (s, 1H), 8.11 (s, 1H), 8.07 (d, 1H), 7.55 (s, 1H), 5.27 (d, 1H), 4.21 (m, 2H), 4.01 (m, 1H), 3.45 (m, 2H), 3.31 (s, 3H); MS (D) for C₂₀H₁₄Cl₃F₃N₄O₄, found 537.0 (MH+).

3-[(2,5-Dichloro-4-{5-[8-chloro-6-(trifluoromethyl)imidazo[1,2-a]pyridin-2-yl]-1,2,4-oxadiazol-3-yl}phenyl)oxy]-2-hydroxy-2-methylpropanoic acid. ¹H-NMR (400 MHz, DMSO-d₆) δ 9.33 (m, 1H), 9.07 (s, 1H), 8.08 (s, 1H), 8.07 (d, 1H), 7.57 (s, 1H), 4.27 (m, 2H), 1.36 (s, 3H); MS (D) for C₂₀H₁₂Cl₃F₃N₄O₅, found 551.0 (MH+).

Methyl 3-[(2,5-Dichloro-4-{5-[8-chloro-6-(trifluoromethyl)imidazo[1,2-a]pyridin-2-yl]-1,2,4-oxadiazol-3-yl}phenyl)oxy]-2-hydroxy-2-methylpropanoate. ¹H-NMR (400 MHz, DMSO-d₆) δ 9.33 (m, 1H), 9.07 (s, 1H), 8.09 (s, 1H), 8.07 (d, 1H), 7.95 (s, 1H), 5.83 (s, 1H), 4.31 (m, 2H), 3.68 (s, 3H), 1.43 (s, 3H); MS (D) for C₂₁H₁₄Cl₃F₃N₄O₅, found 565.0 (MH+).

(2S)-1-[(4-{5-[8-Bromo-6-(trifluoromethyl)imidazo[1,2-a]pyridin-2-yl]-1,2,4-oxadiazol-3yl}-2,5-dichlorophenyl)oxy]propan-2-ol. ¹H-NMR (400 MHz, CDCl₃) δ 8.61 (m, 1H), 8.57 (s, 1H), 8.23 (s, 1H), 7.76 (ds, 1H), 7.10 (s, 1H), 4.31 (dt, 1H), 4.09 (dd, 1H), 3.93 (dd, 1H), 1.35 (d, 3H); MS (D) for C₁₉H₁₂BrCl₂F₃N₄O₃, found 552.9 (M+H).

1-[(4-{5-[8-Bromo-6-(trifluoromethyl)imidazo[1,2-a]pyridin-2-yl]-1,2,4-oxadiazol-3- yl}-2,5-dichlorophenyl)oxy]propan-2-ol. ¹H-NMR (400 MHz, CDCl₃) δ 8.61 (m, 1H), 8.57 (s, 1H), 8.23 (s, 1H), 7.76 (d, 1H), 7.10 (s, 1H), 4.31 (dt, 1H), 4.09 (dd, 1H), 3.93 (dd, 1H), 1.35 (d, 3H); MS (EI) for C₁₉H₁₂BrCl₂F₃N₄O₃, found 552.9 (M+H).

(2R)-1-[(4-{5-[8-Bromo-6-(trifluoromethyl)imidazo[1,2-a]pyridin-2-yl]-1,2,4-oxadiazol-3yl}-2,5-dichlorophenyl)oxy]propan-2-ol. ¹H-NMR (400 MHz, CDCl₃) δ 8.61 (m, 1H), 8.57 (s, 1H), 8.23 (s, 1H), 7.76 (d, 1H), 7.10 (s, 1H), 4.31 (dt, 1H), 4.09 (dd, 1H), 3.93 (dd, 1H), 1.35 (d, 3H); MS (EI) for C₁₉H₁₂BrCl₂F₃N₄O₃, found 553.0 (M+H).

### Example 45

### 3-[(2,5-Dichloro-4-{5-[8-chloro-6-(trifluoromethyl)imidazo[1,2-a]pyridin-2-yl]-1,2,4-oxadiazol-3-yl}phenyl)oxy]-1,1,1-trifluoropropan-2-one

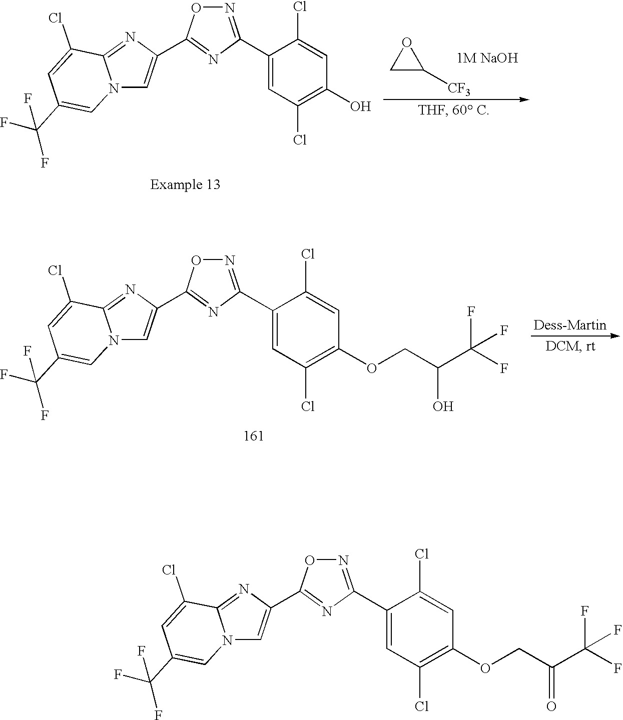

3-[(2,5-Dichloro-4-{5-[8-chloro-6-(trifluoromethyl)imidazo[1,2-a]pyridin-2-yl]-1,2,4-oxadiazol-3-yl}phenyl)oxy]-1,1,1-trifluoropropan-2-ol (161). 2,5-Dichloro-4-(5-(8-chloro-6-(trifluoromethyl)imidazo(1,2-a)pyridine-2-yl)-1,2,4-oxadiazole-3-yl) phenol (500 mg, 1.0 mmol), prepared as described in Example 13, and 1,1,1-trifluoro-2,3-epoxypropane (560 mg, 5.0 mmol) were mixed in THF (2 mL) and 1 M NaOH (1 mL, 1.0 mmol). The resulting solution was stirred at 60° C. for 12 h. After it was cooled to rt, the mixture was extracted with EtOAc. The organic phase was washed with water and brine. The organic layers were combined and dried over Na₂SO₄. Removal of the solvents and purification by flash column chromatography gave 161 (700 mg, 62% yield). ¹H-NMR (400 MHz, DMSO-d₆) δ 9.34 (s, 1H), 9.08 (s, 1H), 8.12 (s, 1H), 8.08 (d, 1H), 7.67 (s, 1H), 6.82 (m, 1H), 4.49 (m, 2H), 4.37 (m, 1H); MS (EI) for C₁₉H₉Cl₃F₆N₄O₃, found 561.0 (MH+).

3-[(2,5-Dichloro-4-{5-[8-chloro-6-(trifluoromethyl)imidazo[1,2-a]pyridin-2-yl]-1,2,4-oxadiazol-3-yl}phenyl)oxy]-1,1,1-trifluoropropan-2-one. 161 (100 mg, 0.18 mmol) was dissolved in THF (2 mL), to which was added a solution of the Dess-Martin periodinane (15 wt % in DCM, 1.5 mL, 0.53 mmol). The resulting mixture was stirred at rt for 4 h. The solvents were removed, and the residue was purified by flash column chromatography to give the title compound (36 mg, 37% yield). ¹H-NMR (400 MHz, DMSO-d₆) δ 9.33 (m, 1H), 9.07 (s, 1H), 8.09 (s, 1H), 8.07 (d, 1H), 7.62 (s, 1H), 7.50 (s, 2H), 4.32 (s, 2H); MS (EI) for C₁₉H₇Cl₃F₆N₄O₃, found 559.0 (MH+).

### Example 46

### (1S)-2-[(2,5-Dichloro-4-{5-[8-chloro-6-(trifluoromethyl)imidazo[1,2-a]pyridin-2-yl]-1,2,4-oxadiazol-3-yl}phenyl)oxy]-1-methylethyl hydrogen sulfate

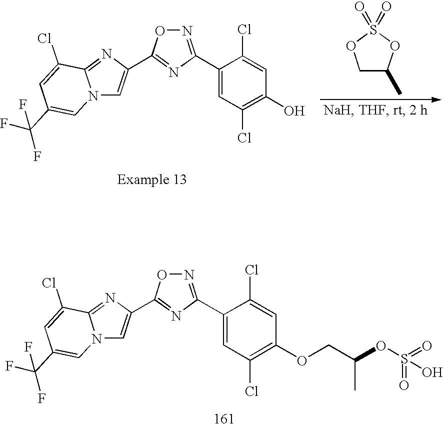

(1S)-2-[(2,5-Dichloro-4-{5-[8-chloro-6-(trifluoromethyl)imidazo[1,2-a]pyridin-2-yl]-1,2,4-oxadiazol-3-yl}phenyl)oxy]-1-methylethyl hydrogen sulfate. To a solution of 2,5-dichloro-4-(5-(8-chloro-6-(trifluoromethyl)imidazo(1,2-a)pyridine-2-yl)-1,2,4-oxadiazole-3-yl) phenol (500 mg, 1.0 mmol), prepared as described in Example 13, in 1:1 THF/DMF (4 mL) was added NaH (60%, 60 mg, 1.5 mmol). The resulting solution was stirred at rt for 15 min. (S)-1,2-propanediol cyclic sulfate (276 mg, 2.0 mmol) was then added. The reaction mixture was stirred at rt for 2 h. Filtration and purification by HPLC gave the title compound (50 mg, 8.5% yield). ¹H-NMR (400 MHz, DMSO-d₆) δ 9.33 (m, 1H), 9.08 (s, 1H), 8.10 (s, 1H), 8.06 (d, 1H), 7.58 (s, 1H), 4.45 (m, 1H), 4.28 (m, 2H), 1.31 (d, 3H) MS (EI) for C₁₉H₁₂Cl₃F₃N₄O₆S, found 587.0 (MH−).

### Example 47

### 2-Amino-3-[(5-chloro-4-{5-[8-chloro-6-(trifluoromethyl)imidazo[1,2-a]pyridin-2-yl]-1,3,4-thiadiazol-2-yl}-2-fluorophenyl)oxy]propan-1-ol

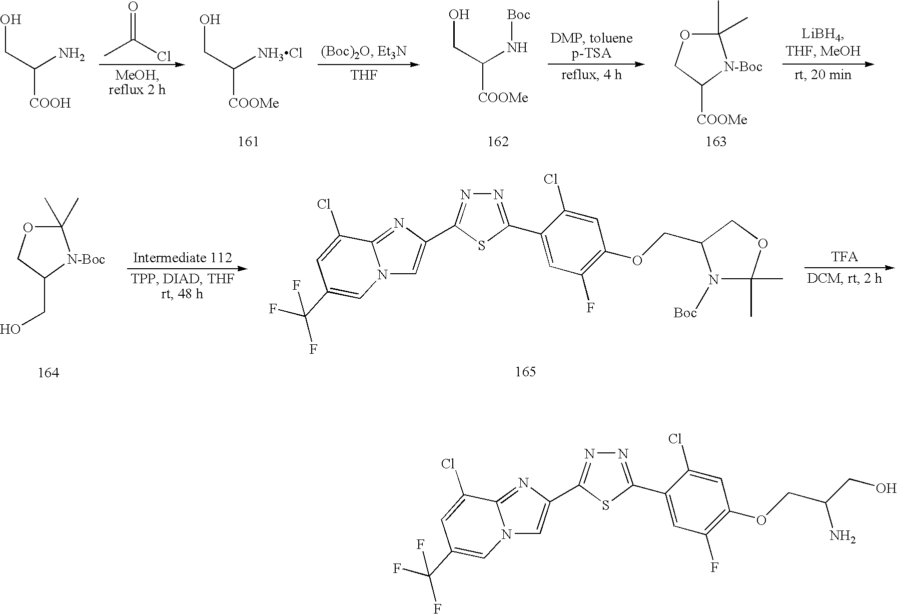

Methyl 2-amino-3-hydroxypropanoate hydrochloride salt (161). Acetyl chloride (57.5 mL, 806 mmol) was added dropwise over a period of 10 min to stirring MeOH (375 mL) at rt under N₂ atmosphere. The solution was stirred for a further 5 min, then solid D,L-serine (30 g, 286 mmol) was added in one portion and the solution was slowly heated to reflux. The refluxing was continued for 2 hr, then the solution was allowed to cool to room temperature and the solvent was removed under reduced pressure to give 43 g of crude 161 as a white crystalline solid which was used without further purification.

Methyl 2-(tert-butoxycarbonylamino)-3-hydroxypropanoate (162). To a stirring solution of 161 (43 g, 275.6 mmol) THF (246 mL) and triethylamine (34.4 mL, 247 mmol) was added solution of di-tert-butyl dicarbonate (35.13 g, 161.1 mmol) dropwise over a period of 1 hr at 0° C. After 10 min of additional stirring, the ice-water bath was removed and the suspension was stirred for 14 h at room temperature, then warmed at 50° C. for a further 3 h. The solvent was removed under reduced pressure and the residue was partitioned between diethyl ether (200 mL) and saturated aqueous bicarbonate solution (250 mL). The aqueous phase was extracted with three 150-mL portions of diethyl ether. The combined organic phases were dried with anhydrous sodium sulfate and concentrated under reduced pressure to give 25 g of 162 as colorless oil that was used without further purification.

3-tent-Butyl 4-methyl 2,2-dimethyloxazolidine-3,4-dicarboxylate (163). To a solution of 162 (25 g, 114.15 mmol) in toluene (250 mL) is added 2,2-dimethoxypropane (13 mL, 106 mmol) and p-TSA (0.12 g). The resulting solution was refluxed for 4 h. After completion, the solvent was removed under reduced pressure. Water was added to the residual mass and it was extracted with ethyl acetate. The combined organic phases were dried with anhydrous sodium sulfate and concentrated under reduced pressure and chromatographed (EtOAc:Hexane 1:9) to give 21 g of 163 as a pale yellow oil. The product was used without further purification.

tert-Butyl 4-(hydroxymethyl)-2,2-dimethyloxazolidine-3-carboxylate (164). To a stirring solution of 163 (21 g, 81.1 mmol) in THF (520 mL) and MeOH (30 mL) was added LiBH₄ (3.56 g, 163.40 mmol) portion wise over 20 min at 0° C. and the suspension was stirred for an additional 20 min, when TLC analysis showed the complete formation of the alcohol 164. The reaction mixture was cooled and ice-water was added and stirred for 10 min. Solvent was removed, water was added, and the mixture was extracted with EtOAc. The combined organic layers were dried with anhydrous sodium sulfate and concentrated under reduced pressure. The crude compound was chromatographed (EtOAc:Hexane 1:19) giving compound 164 (16 g, 86% crude yield).

tert-Butyl 4-((5-chloro-4-(5-(8-chloro-6-(trifluoromethyl)imidazo[1,2-a]pyridin-2-yl)-1,3,4-thiadiazol-2-yl)-2-fluorophenoxy)methyl)-2,2-dimethyloxazolidine-3-carboxylate (165). To a stirring solution of intermediate 112 (1.0 g, 2.22 mmol) and 164 (0.56 g, 2.4 mmol) in THF (10 mL) was added Ph₃P (0.88 g, 3.33 mmol) and the reaction was cooled to 0° C. DIAD (0.674 g, 3.33 mmol) was added dropwise to the reaction mixture and the temperature was brought to rt in 10 min. The reaction was later stirred for 48 h. After completion, solvent was removed in vacuo and diethyl ether was added to precipitate out the product as a white solid, which was filtered giving ether wash and dried to give compound 165 (0.65 g, 44% yield) which was used as such for the next step.

Amino-3-[(5-chloro-4-{5-[8-chloro-6-(trifluoromethyl)imidazo[1,2-a]pyridin-2-yl]-1,3,4-thiadiazol-2yl}-2-fluorophenyl)oxy]propan-1-ol. Compound 165 (0.65 g, 0.98 mmol) was dissolved in a mixture of TFA: DCM (3:7, 20 mL) cooled at 0° C. and then reaction mixture was stirred for 2 h at rt. Solvent was removed under reduced pressure, and the compound was purified by preparative HPLC to give 2-amino-3-[(5-chloro-4-{5-[8-chloro-6-(trifluoromethyl)imidazo[1,2-a]pyridin-2-yl]-1,3,4-thiadiazol-2-yl}-2-fluorophenyl)oxy]propan-1-ol (0.1 g, 20% yield). ¹H-NMR (400 MHz, DMSO-d₆) δ 9.32 (s, 1H), 8.91 (s, 1H), 8.20 (m, 1H), 8.18 (br s, 2H), 7.99 (s, 1H), 7.66 (d, 1H), 5.47 (t, 1H), 4.39 (m, 2H), 3.68 (m, 3H); MS (EI) for C₁₉H₁₃Cl₂F₄N₅O₂S, found 522 (MH+).

The following compounds were prepared using the same or analogous synthetic techniques in Example 47 and substituting with appropriate reagents, which were commercially available or were prepared using procedures herein or procedures known to one of ordinary skill in the art.

(2R)-2-Amino-3-[(5-chloro-4-{5-[8-chloro-6-(trifluoromethyl)imidazo[1,2-a]pyridin-2-yl]-1,3,4-thiadiazol-2yl}-2-fluorophenyl)oxy]propan-1-ol. ¹H-NMR (400 MHz, DMSO-d₆) δ 9.33 (s, 1H), 9.09 (s, 1H), 8.23 (br s, 3H), 8.22 (d, 1H), 8.01 (s, 1H), 7.68 (d, 1H), 5.47 (t, 1H), 4.38 (m, 2H), 3.67 (m, 3H). MS(EI) for C₁₉H₁₃Cl₂F₄N₅O₂S, found 522.0 (MH+).

(2S)-2-Amino-3-[(5-chloro-4-{5-[8-chloro-6-(trifluoromethyl)imidazo[1,2-a]pyridin-2-yl]-1,3,4-thiadiazol-2yl}-2-fluorophenyl)oxy]propan-1-ol. ¹H-NMR (400 MHz, DMSO-d₆) δ 9.35 (s, 1H), 8.93 (s, 1H), 8.31 (Br. s, 2H), 8.22-8.19 (d, 1H), 8.01 (s, 1H), 7.69-7.67 (d, 1H), 5.5 (Br. s, 1H), 4.46-4.43 (m, 1H), 4.40-4.36 (m, 1H), 3.77-3.73 (m, 1H), 3.71-3.67 (m, 1H), 3.59 (m, 1H). MS(EI) for C₁₉H₁₃Cl₂F₄N₅O₂S, found 522.0 (MH+).

(2R)-2-Amino-3-[(2,5-dichloro-4-{5-[8-chloro-6-(trifluoromethyl)imidazo[1,2-a]pyridin-2-yl]-1,3,4-thiadiazol-2-yl}phenyl)oxy]propan-1-ol. ¹H-NMR (400 MHz, DMSO-d₆) δ 9.35 (s, 1H), 8.92 (s, 1H), 8.39 (s, 1H), 8.19 (br s, 2H), 8.00 (s, 1H), 7.62 (s, 1H), 5.45 (t, 1H), 4.40 (m, 2H), 3.75 (m, 2H), 3.62 (m, 1H). MS(EI) for C₁₉H₁₃C₁₃F₃N₅O₂S, found 537.8 (MH+).

2-Amino-3-[(5-chloro-2-fluoro-4-{5-[7-(trifluoromethyl)imidazo[1,2-a]pyridin-2-yl]-1,3,4-thiadiazol-2-yl}phenyl)oxy]propan-1-ol hydrochloride salt. ¹H-NMR (400 MHz, TFA) δ 9.00 (m, 2H), 8.42 (s, 1H), 8.20 (d, 1H), 7.81 (d, 1H), 7.35 (d, 1H), 5.00 (m, 2H), 4.72 (m, 2H), 4.51 (m, 1H). MS(EI) for C₁₉H₁₄ClF₄N₅O₂S, found 488.1 (MH+).

### Example 48

### 1-[(2,5-Dichloro-4-{5-[8-chloro-6-(trifluoromethyl)imidazo[1,2-a]pyridin-2-yl]-1,2,4-oxadiazol-3-yl}phenyl)oxy]-3-hydroxypropan-2-one

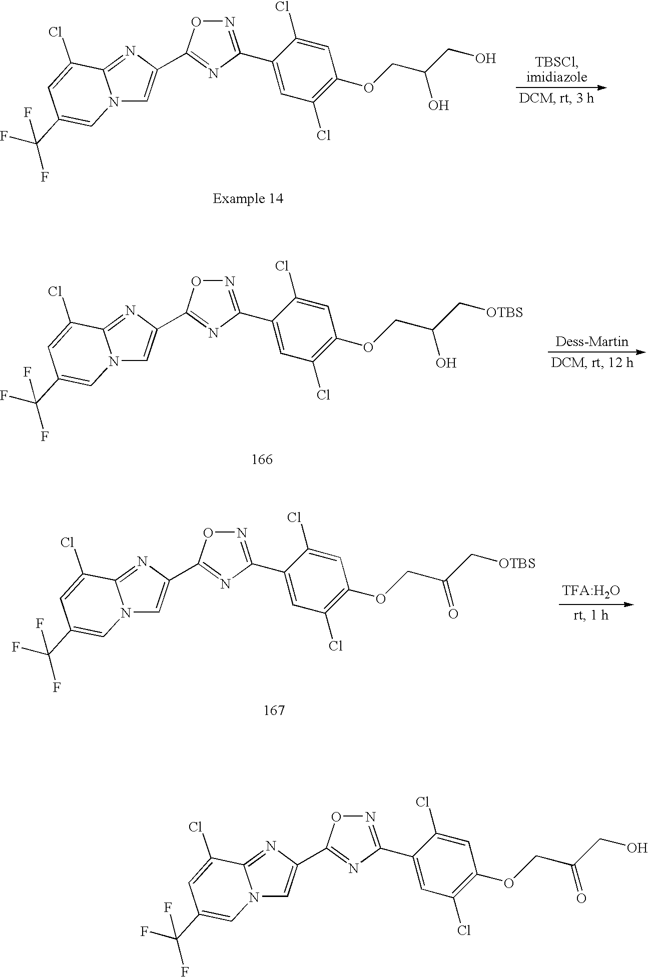

1-(tert-Butyldimethylsilyloxy)-3-(2,5-dichloro-4-(5-(8-chloro-6-(trifluoromethyl)imidazo[1,2-a]pyridin-2-yl)-1,2,4-oxadiazol-3-yl)phenoxy)propan-2-ol (166). To a stirred solution of 3-(2,5-dichloro-4-(5-(8-chloro-6-(trifluoromethyl)imidazo[1,2-a]pyridin-2-yl)-1,2,4-oxadiazol-3-yl)phenoxy)propane-1,2-diol, made as outlined in Example 14, (0.48 g, 0.919 mmol), imidazole (0.187 g, 2.75 mmol) and DMAP (0.44 g) in dry DCM was added TBSCl (0.165 g 1.1 mmol) at 40° C. and the reaction mixture was stirred for 3 h at rt. Water was added and the reaction mixture was extracted in DCM. The organic layer were dried over MgSO₄, filtered, concentrated and chromatograhed (50% EtOAc, Hexane) to provide (0.2 g, 34.48%) of pure compound 166.

1-(tert-butyldimethylsilyloxy)-3-(2,5-dichloro-4-(5-(8-chloro-6-(trifluoromethyl)imidazo[1,2-a]pyridin-2-yl)-1,2,4-oxadiazol-3-yl)phenoxy)propan-2-one (167). To a stirred solution of 166 (0.2 g, 0.315 mmol) in DCM was added Dess-martin periodinane (0.214 g, 0.504 mmol) and the reaction mixture was stirred at rt for 12 hours. The reaction mixture was basified with a 1:1 aq solution of sodium thiosulphate and sodium bicarbonate, and the reaction mixture was extracted with ethyl acetate. The organic layer was dried over MgSO₄, filtered, and concentrated to obtain 0.15 g of 167 as crude product which was carried forward as such for the next step.

1-[(2,5-Dichloro-4-{5-[8-chloro-6-(trifluoromethyl)imidazo[1,2-a]pyridin-2-yl]-1,2,4-oxadiazol-3-yl}phenyl)oxy]-3-hydroxypropan-2-one. A reaction mixture of 167 (0.15 g, 0.23 mmol) in TFA: water (9:1, 10 mL) was stirred for 1 h at rt. The solvent was removed and the crude mass was submitted for preparative HPLC to obtain 1-[(2,5-dichloro-4-{5-[8-chloro-6-(trifluoromethyl)imidazo[1,2-a]pyridin-2-yl]-1,2,4-oxadiazol-3-yl}phenyl)oxy]-3-hydroxypropan-2-one (0.02 g, 17% yield) as pure compound. ¹H NMR (400 MHz, DMSO-d₆,) δ 9.4 (s, 1H), 9.1 (s, 1H), 8.1 (s, 1H), 8.0 (s, 1H), 7.4 (s, 1H), 5.3 (s, 2H), 4.3 (s, 2H); MS (EI) for C₁₉H₁₀Cl₃F₃N₄O₄, found 521 (MH+).

### Example 49

### 3-[(2,5-Dichloro-4-{5-[8-chloro-6-(trifluoromethyl)imidazo[1,2-a]pyridin-2-yl]-1,2,4-oxadiazol-3-yl}phenyl)oxy]-2,2-difluoropropan-1-ol

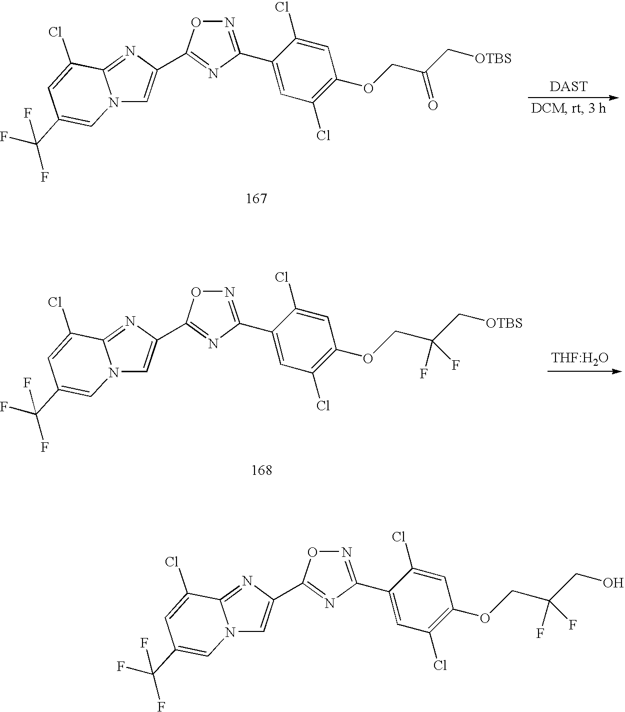

3-(4-(3-(tert-Butyldimethylsilyloxy)-2,2-difluoropropoxy)-2,5-dichlorophenyl)-5-(8-chloro-6-(trifluoromethyl)imidazo[1,2-a]pyridin-2-yl)-1,2,4-oxadiazole (168). To a stirred solution of 167 (0.5 g, 0.79 mmol) in DCM (20 mL) was added DAST (2.5 mL) at rt and the reaction mixture was stirred for 3 h. Later, it was quenched with NaHCO₃ solution and extracted with DCM. The organic layer was dried over MgSO₄, concentrated to obtain 0.530 g of 168 as crude product which was carried forward as such for the next step.

3-[(2,5-Dichloro-4-{5-[8-chloro-6-(trifluoromethyl)imidazo[1,2-a]pyridin-2-yl]-1,2,4-oxadiazol-3-yl}phenyl)oxy]-2,2-difluoropropan-1-ol. A reaction mixture of 168 (0.40 g, 0.6 mmol) in TFA: water (9:1, 20 mL) was stirred for 3 h at rt. Solvent was removed and the crude mass was submitted for the preparative HPLC to obtain 3-[(2,5-dichloro-4-{5-[8-chloro-6-(trifluoromethyl)imidazo[1,2-a]pyridin-2-yl]-1,2,4-oxadiazol-3-yl}phenyl)oxy]-2,2-difluoropropan-1-ol (0.1 g, 31% yield) as pure compound. ¹H NMR (400 MHz , DMSO-d₆) δ 9.4 (s, 1H), 9.0 (s, 1H), 8.1 (s, 1H), 8.0 (s, 1H), 7.7 (s, 1H), 5.8 (bs, 1H), 4.6 (t, 2H), 3.8 (t, 2H); MS (EI) for C₁₉H₁₀Cl₃F₅N₄O₃, found 543 (MH+).

### Example 50

### 3-[(2,5-Dichloro-4-{5-[8-chloro-6-(trifluoromethyl)imidazo[1,2-a]pyridin-2-yl]-1,2,4-oxadiazol-3-yl}phenyl)oxy]-2-methylpropane-1,2-diol

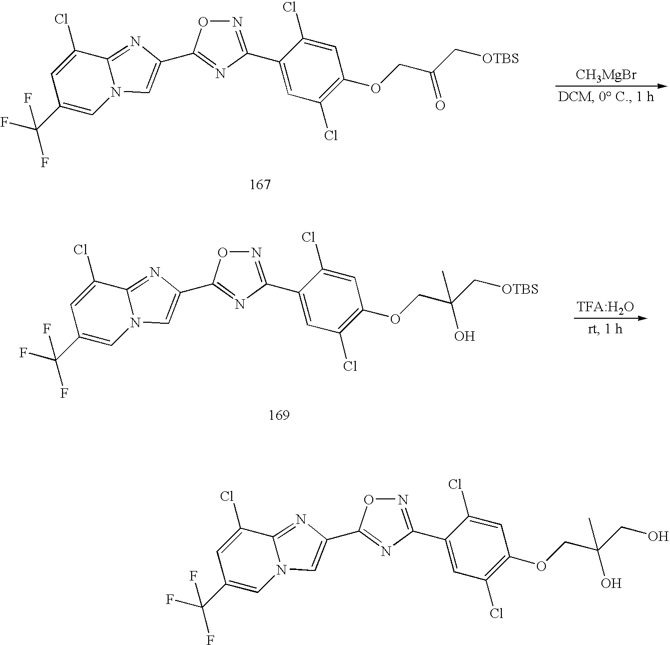

1-(tert-Butyldimethylsilyloxy)-3-(2,5-dichloro-4-(5-(8-chloro-6-(trifluoromethyl)imidazo[1,2-a]pyridin-2-yl)-1,2,4-oxadiazol-3-yl)phenoxy)-2-methylpropan-2-ol (169). To a stirred solution of compound 167 (0.4 g, 0.63 mmol) in DCM at 0° C. was added a solution of 3 M CH₃MgBr in THF (0.42 mL) dropwise and the reaction was stirred for 1 h at 0° C., after which it was quenched with ammonium chloride solution, followed by brine. The organic extract was dried over Na₂SO₄, filtered, and concentrated. The resulting solids were washed with pentane to yield 0.410 g of 169 as a solid compound which was used in the next step without further purification.

3-[(2,5-Dichloro-4-{5-[8-chloro-6-(trifluoromethyl)imidazo[1,2-a]pyridin-2-yl]-1,2,4-oxadiazol-3-yl}phenyl)oxy]-2-methylpropane-1,2-diol. Compound 169 (0.41 g, 0.63 mmol) was dissolved in DCM (2 mL) and cooled to 0° C. to which was added 5 mL of TFA/DCM (1:1) and stirred at 0° C. for 5 min and later at rt for 1 h. Solvent was removed under reduced pressure, and the compound was purified by preperative HPLC to give (0.15 g, 45%) of 3-[(2,5-dichloro-4-{5-[8-chloro-6-(trifluoromethyl)imidazo[1,2-a]pyridin-2-yl]-1,2,4-oxadiazol-3-yl}phenyl)oxy]-2-methylpropane-1,2-diol as a white solid. ¹H NMR (400 MHz, DMSO-d₆) δ 9.4 (s, 1H), 9.1 (s, 1H), 8.1 (s, 1H), 8.0 (s, 1H), 7.6 (s, 1H), 4.1 (d, 1H), 4.0 (d, 1H), 3.5 (d, 1H), 3.4 (d, 1H), 1.2 (s, 3H); MS (EI) for C₂₀H₁₄Cl₃F₃N₄O₄, found 537 (MH+).

### Example 51

### 2-[(2,5-Dichloro-4-{5-[8-chloro-6-(trifluoromethyl)imidazo[1,2-a]pyridin-2-yl]-1,2,4-oxadiazol-3-yl}phenyl)oxy]propane-1,3-diol

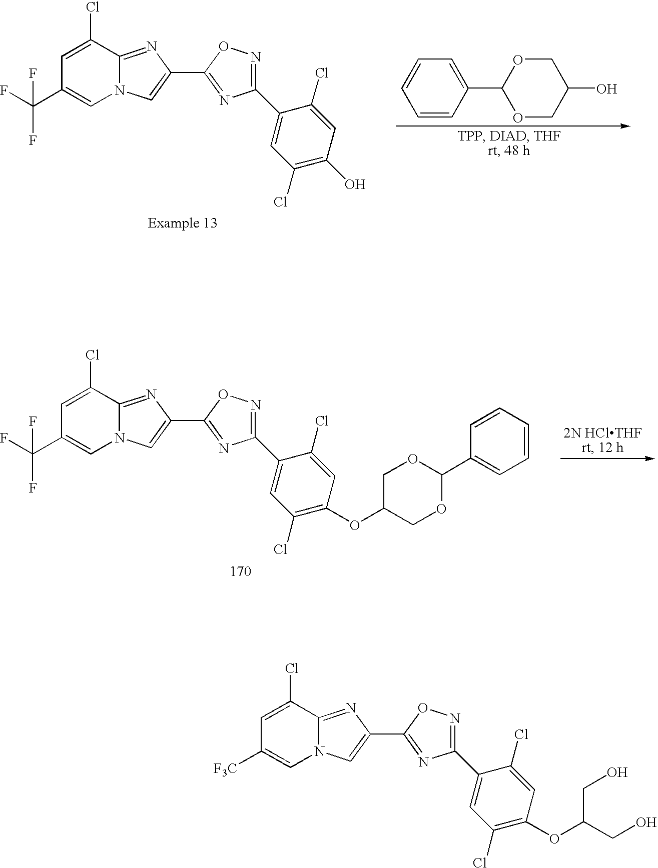

5-(8-Chloro-6-(trifluoromethyl)imidazo[1,2-a]pyridin-2-yl)-3-(2,5-dichloro-4-(2-phenyl-1,3-dioxan-5-yloxy)phenyl)-1,2,4-oxadiazole (170). To a stirring solution of 2,5-dichloro-4-(5-(8-chloro-6-(trifluoromethyl)imidazo(1,2-a)pyridine-2-yl)-1,2,4-oxadiazole-3-yl)phenol, made as outlined in Example 13, (1.0 g, 2.22 mmol) and 2-phenyl-1,3-dioxan-5-ol (0.6 g, 3.33 mmol) in THF (15 mL) was added triphenyl phosphine (1.2 g, 4.44 mmol) and the reaction was cooled to 0° C. DIAD (0.9 g, 4.44 mmol) was added dropwise to the reaction mixture and the temperature was brought to rt in 10 min with subsequent stirring for 48 h. Solvent was removed in vacuo and diethyl ether was added to precipitate out the product as a white solid, which was filtered, washed with ether and dried to give compound 170 (0.7 g) which was used as such for the next step.

2-[(2,5-Dichloro-4-{5-[8-chloro-6-(trifluoromethyl)imidazo[1,2-a]pyridin-2-yl]-1,2,4-oxadiazol-3-yl}phenyl)oxy]propane-1,3-diol. To a stirred solution of 170 (0.7 g, 1.14 mmol) in THF (17.5 mL) was added 2 N HCl (17.5 mL) at rt and the reaction mixture was stirred overnight. Later, the reaction was basified with saturated solutions of both K₂CO₃, NaHCO₃. The reaction was extracted with chloroform. The organic extracts were dried over Na₂SO₄, filtered, concentrated and purified by prep HPLC to afford of 2-[(2,5-dichloro-4-}548-chloro-6-(trifluoromethyl)imidazo[1,2-a]pyridin-2-yl]-1,2,4-oxadiazol-3-yl}phenyl)oxy]propane-1,3-diol in (0.065 g, 11% yield). ¹H NMR (400 MHz, DMSO-d₆) δ 9.4 (s, 1H), 9.1 (s, 1H), 8.1 (d, 2H) 7.8 (s, 1H), 4.8 (bs, 2H), 4.6 (m, 1H), 3.6-3.88 (m, 2H); MS (EI) for C₁₉H₁₂Cl₃F₃N₄O₄, found 523 (MH+).

### Example 52

### (2E)-3-(3-Chloro-4-{5-[8-chloro-6-(trifluoromethyl)imidazo[1,2-a]pyridin-2-yl]-1,2,4-oxadiazol-3-yl}phenyl)prop-2-enoic acid

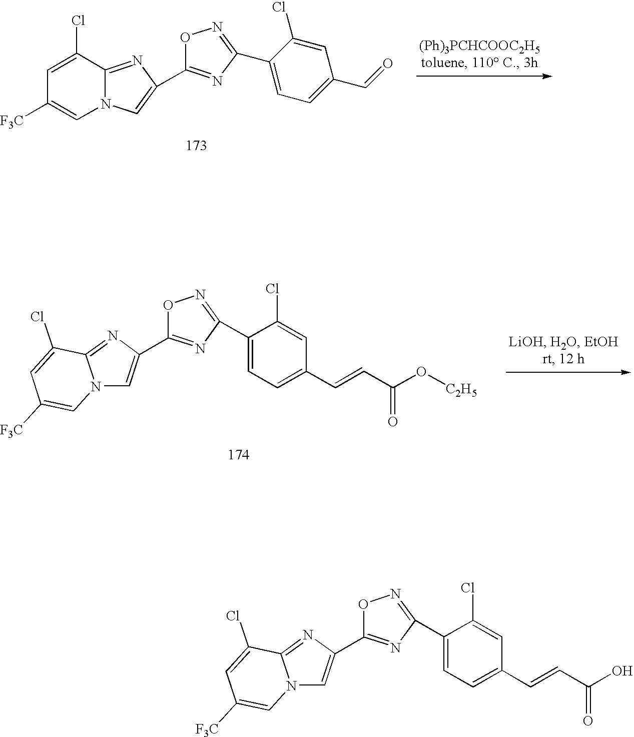

(E)-Ethyl 3-(3-chloro-4-(5-(8-chloro-6-(trifluoromethyl)imidazo[1,2-a]pyridin-2-yl)-1,2,4-oxadiazol-3-yl)phenyl)acrylate (174). Aldehyde 173 was synthesized in the same manner as intermediate 40 in Example 5, substituting with appropriate reagents. A solution of aldehyde 173 (1.0 g, 2.34 mmol) and (ethoxycarbonylmethylen)-triphenyl phosphoran (2.5 g, 7.4 mmol) in toluene (20 mL) was refluxed for 3 h. Reaction mixture was cooled, diluted with EtOAc and washed with water and brine. The organic layer was dried over Na₂SO₄, filtered, concentrated and chromatographed (EtOAc:Hexane 1:9) to afford 174 as white solid (0.6 g, 52% yield).

(2E)-3-(3-Chloro-4-{5-[8-chloro-6-(trifluoromethyl)imidazo[1,2-a]pyridin-2-yl]-1,2,4-oxadiazol-3-yl}phenyl)prop-2-enoic acid. To a stirred solution of 174 (0.25 g, 0.5 mmol) in EtOH (3 mL) was added an aq solution of 2 N LiOH (0.65 mL) and the reaction mixture was stirred at rt for 12 h. The reaction mixture was cooled to 0° C. and neutralized with 1 N HCl. A white solid precipitated out which was filtered, washed with isopropanol and dried to give (2E)-3-(3-chloro-4-{5-[8-chloro-6-(trifluoromethyl)imidazo[1,2-a]pyridin-2-yl]-1,2,4-oxadiazol-3-yl}phenyl)prop-2-enoic acid. (0.05 g, 22% yield). ¹H NMR (400 MHz, DMSO-d₆) δ 9.3 (s, 1H), 9.1 (s, 1H), 8.1 (m, 3H), 7.9 (m, 1H), 7.7 (m, 1H), 7.6 (d, 1H), 6.8 (d, 1H); MS (EI) for C₁₉H₉Cl₂F₃N₄O₃, found 469 (MH+).

Using the same or analogous synthetic techniques in Example 52 and substituting with appropriate reagents (prepared using procedures described herein), the following compound was prepared. (2E)-3-(5-Chloro-4-{5-[8-chloro-6-(trifluoromethyl)imidazo[1,2-a]pyridin-2-yl]-1,2,4-oxadiazol-3yl}-2-fluorophenyl)prop-2-enoic acid. ¹H-NMR (400 MHz, DMSO-d₆) δ 12.82 (s, 1H), 9.33 (s, 1H), 9.05 (s, 1H), 8.27 (d, 1H), 8.05 (s, 1H), 7.95 (d, 1H), 7.62 (2, 1H), 6.84 (d, 1H); MS (EI) for C₁₉H₈C₁₂F₄N₄O₃, found 485 (MH−).

### Example 53

### 3-[(3-Chloro-4-{5-[8-chloro-6-(trifluoromethyl)imidazo[1,2-a]pyridin-2-yl]-1,2,4-oxadiazol-3-yl}phenyl)methyl]-1,2,4-oxadiazol-5(4H)-one

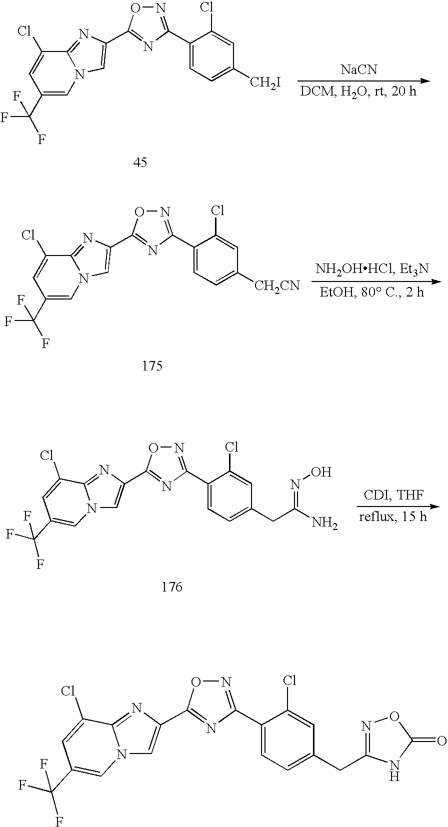

2-(3-Chloro-4-(5-(8-chloro-6-(trifluoromethyl)imidazo[1,2-a]pyridin-2-yl)-1,2,4-oxadiazol-3-yl)phenyl)acetonitrile (175). To a stirred solution of 45 (2.0 g, 3.7 mmol), synthesized as outlined in Example 7, in DCM:Water (1:1) (30 mL), was added NaCN (0.546 g, 11.5 mmol) at rt and stirred for 20 h. DCM was added and the resulting mixture washed with Aq NaHCO₃. The organic layer was dried over Na₂SO₄, filtered, concentrated and chromatographed, (EtOAc/Hexane 3:7) giving 175 (0.7 g, 43% yield).

2-(3-Chloro-4-(5-(8-chloro-6-(trifluoromethyl)imidazo[1,2-a]pyridin-2-yl)-1,2,4-oxadiazol-3-yl)phenyl)-N′-hydroxyacetimidamide (176). To an ethanolic solution (7 mL) of hydroxyl amine hydrochloride (0.572 g, 8.2 mol), triethylamine (1.33 mL, 9.5 mol) was added slowly and the mixture stirred at room temperature for 1 h. Compound 175 (0.6 g, 1.36 mole) was added, followed by stirring at room temperature for 0.5 h, then heated to 80° C. for 2 h. The reaction mixture was concentrated in vacuo to remove ethanol and extracted with ethyl acetate. The combined organic fractions were washed with water and brine, dried over Na₂SO₄, and concentrated to give 176 (0.15 g).

3-[(3-Chloro-4-{5-[8-chloro-6-(trifluoromethyl)imidazo[1,2-a]pyridin-2-yl]-1,2,4-oxadiazol-3-yl}phenyl)methyl]-1,2,4-oxadiazol-5(4H)-one. To a stirred solution of 176 (0.1 g, 0.212 mmol) in THF (5 mL) was added CDI (0.068 g, 0.42 mmol) and the reaction was stirred at rt for 15 min followed by refluxing for 15 h. Solvent was removed under reduced pressure, water was added and it was extracted with EtOAc. Organic phases were washed with brine, dried over Na₂SO₄, filtered, concentrated and the crude mass was purified by prep HPLC to give 3-[(3-chloro-4-{5-[8-chloro-6-(trifluoromethyl)imidazo[1,2-a]pyridin-2-yl]-1,2,4-oxadiazol-3-yl}phenyl)methyl]-1,2,4-oxadiazol-5(4H)-one (0.03 g, 29% yield). ¹H NMR (400 MHz, DMSO-d₆) δ 12.4 (s, 1H), 9.4 (s, 1H), 9.0 (s, 1H), 8.1 (m, 2H), 7.7 (s, 1H), 7.5 (d, 1H), 4.1 (s, 2H); MS (EI) for C₁₉H₉Cl₂F₃N₆O₃, found 497 (MH+).

### Example 54

### 3-(3-Chloro-4-{5-[8-chloro-6-(trifluoromethyl)imidazo[1,2-a]pyridin-2-yl]-1,2,4-oxadiazol-3-yl}phenyl)-1,2,4-oxadiazol-5(2H)-one

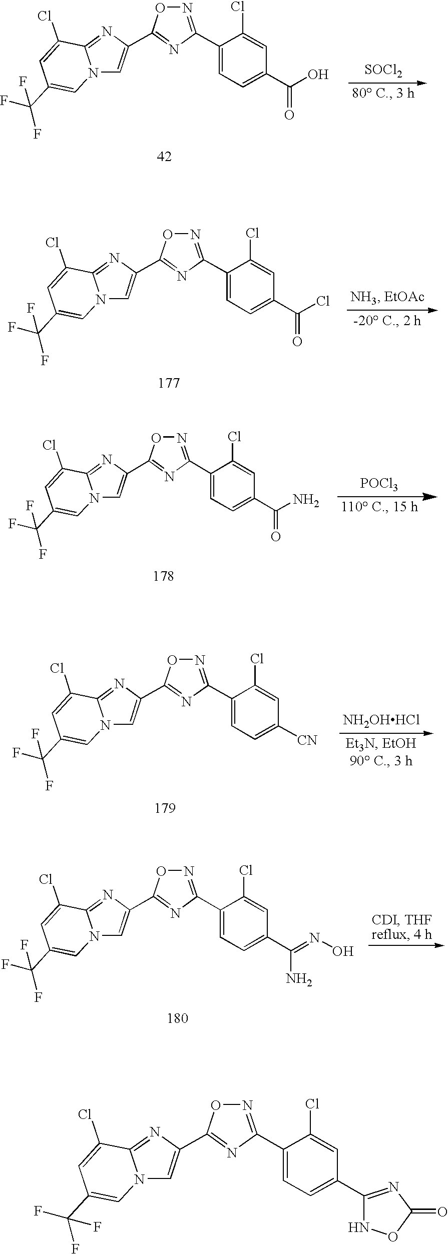

3-Chloro-4-(5-(8-chloro-6-(trifluoromethyl)imidazo[1,2-a]pyridin-2-yl)-1,2,4-oxadiazol-3-yl)benzoyl chloride (177). A stirred mixture of 42 (0.5 g, 1.1 mmol), synthesized as outlined in Example 6, and SOCl₂ (2 mL) was refluxed at 80° C. for 3 h. Solvent was removed to obtain 177 (0.51 g) as a thick oil which was carried forward as such without any purification.

3-Chloro-4-(5-(8-chloro-6-(trifluoromethyl)imidazo[1,2-a]pyridin-2-yl)-1,2,4-oxadiazol-3-yl)benzamide (178). To a stirred solution of 177 (0.51 g, 1.1 mmol) in EtOAc (10 mL) at −20° C., NH₃ was bubbled for 2 h. The reaction mixture was allowed to stir for 1 h to evaporate ammonia from the reaction. Solvent was removed under reduced pressure. A brownish solid was obtained which was filtered and washed with water to obtain 178 (0.3 g, 63% yield).

3-Chloro-4-(5-(8-chloro-6-(trifluoromethyl)imidazo[1,2-a]pyridin-2-yl)-1,2,4-oxadiazol-3-yl)benzonitrile (179). A stirred solution of 178 (1 g, 2.26 mmol) in POCl₃ (10 mL) was refluxed at 110° C. for 15 h. After completion, POCl₃ was removed under reduced pressure and diluted with EtOAc. The organic phase was washed with aq. NaHCO₃, dried over Na₂SO₄ and concentrated to obtain 179 as brownish oil (0.7 g, 73.68% yield).

3-Chloro-4-(5-(8-chloro-6-(trifluoromethyl)imidazo[1,2-a]pyridin-2-yl)-1,2,4-oxadiazol-3-yl)-N′-hydroxybenzimidamide (180). To a stirred solution of hydroxylamine hydrochloride (0.8 g, 11.58 mmol) in ethanol (10 mL) was added triethylamine (1.84 mL, 13.2 mmol) and it was stirred at rt for 30 min and then 179 (0.7 g, 1.65 mmol) was added and the mixture further stirred at rt for 20 min. The reaction mixture was heated to 90° C. for 3 h. After completion of the reaction, solvent was removed in vacuo. The residue was dissolved in ethyl acetate and washed with water, dried over sodium sulphate and concentrated in vacuo to afford of 180 as a white solid (0.6 g, 70% yield).

3-(3-Chloro-4-{5-[8-chloro-6-(trifluoromethyl)imidazo[1,2-a]pyridin-2-yl]-1,2,4-oxadiazol-3-yl}phenyl)-1,2,4-oxadiazol-5(2H)-one. To a stirred solution of 180 (0.5 g, 1.09 mmol) in THF (10 mL) was added CDI (0.444 g, 2.7 mmol) and refluxed for 5 h. Solvent was removed and water was added. The aq layer was extracted with EtOAc. The organic layer was dried over Na₂SO₄, filtered and concentrated. The solid obtained was crystallized from acetonitrile to obtain 3-(3-chloro-4-{5-[8-chloro-6-(trifluoromethyl)imidazo[1,2-a]pyridin-2-yl]-1,2,4-oxadiazol-3-yl}phenyl)-1,2,4-oxadiazol-5(2H)-one as brown solid (0.35 g, 67.30% yield). ¹H NMR (400 MHz, DMSO-d₆) δ 9.4 (s, 1H), 9.1 (s, 1H), 8.0 (m, 4H); MS (EI) for C₁₈H₇Cl₂F₃N₆O₃, found 483 (MH+).

### Example 55

### 3-Chloro-4-{5-[8-chloro-6-(trifluoromethyl)imidazo[1,2-a]pyridin-2-yl]-1,2,4-oxadiazol-3-yl}phenylalanine

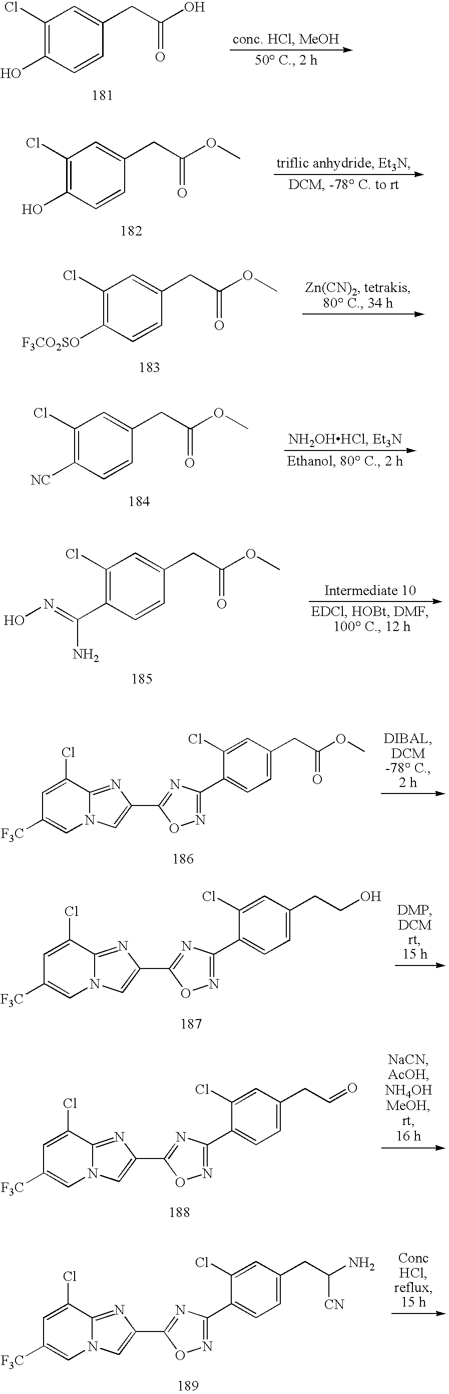

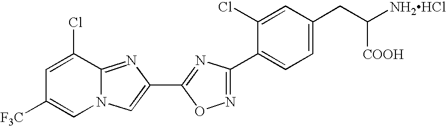

Methyl 2-(3-chloro-4-hydroxyphenyl)acetate (182). To a stirred solution of 181 (20 g, 108 mmol) in MeOH (160 mL) at 0° C. was added conc HCl (15 mL) dropwise. The reaction mixture was heated at 60° C. for 2 h. Solvent was removed under reduced pressure and water was added. The mixture was neutralized with saturated NaHCO₃ solution and extracted with EtOAc. The organic layer was dried over Na₂SO₄, filtered, and concentrated to obtain 182 as colorless oil (17 g, 79% yield) which was used as such in the next step.

Methyl 2-(3-chloro-4-(trifluoromethylsulfonyloxy)phenyl)acetate (183). To a stirred solution of 182 (15 g, 74.6 mmol) and Et₃N (10.3 mL, 74.6 mmol) in DCM (150 mL) was added triflic anhydride (12.5 mL, 74.6 mmol) at −78° C. dropwise over a period of 30 min. The reaction was then further stirred at −78° C. for 2 h. EtOAC was added to reaction mixture and then it was quenched with saturated NH₄Cl solution. The temperature was then brought to rt in 30 min. The slurry was filtered and the organic layer was separated, dried over Na₂SO₄, filtered, and concentrated to give 183 as brownish solid compound (23 g, 93% yield).

Methyl 2-(3-chloro-4-cyanophenyl)acetate (184). To a stirred solution of 183 (16 g, 48 mmol) in DMF (80 mL) was added Zn(CN)₂ (5.6 g, 48 mmol) and tetrakis (5.2 g, 4.6 mmol) at 25° C. The reaction was stirred at 80° C. for 34 h. EtOAc and saturated NaHCO₃ solution were added to the reaction and it was further stirred for 30 min. The reaction mixture was filtered and organic layer was separated. Aqueous layer was further extracted with EtOAc. The combined organic extracts were washed with brine, dried over Na₂SO₄, filtered, concentrated and chromatographed (EtOAc/Hexane 1:9) to obtain 184 as a white solid (8 g, 79% yield).

Methyl 2-(3-chloro-4-(N′-hydroxycarbamimidoyl)phenyl)acetate (185). To a stirred solution of hydroxylamine hydrochloride (18.5 g, 266 mmol) in ethanol (70 mL) was added triethylamine (31 mL, 228 mmol) and it was stirred at rt for 30 min and then 184 (8 g, 38 mmol) was added and further stirred at rt for 20 min. The reaction mixture was heated to 80° C. for 2 h. After completion of the reaction, solvent was removed in vacuo. The residue was dissolved in ethyl acetate and washed with water, dried over sodium sulphate and concentrated in vacuo to afford (5.3 g, 57%) of 185 as a white solid.

Methyl 2-(3-chloro-4-(5-(8-chloro-6-(trifluoromethyl)imidazo[1,2-a]pyridin-2-yl)-1,2,4-oxadiazol-3-yl)phenyl)acetate (186). To a stirred solution of 185 (1.41 g, 5.34 mmol) in DMF (10 mL) was added EDCI (1.02 g, 5.34 mmol) and HOBT (0.72 g, 5.34 mmol) and stirred at rt for 20 min and then 10 (1.0 g, 4.1 mmol) was added and further stirred at rt for 20 min. The reaction mixture was heated to 100° C. for 15 h. The reaction mixture was concentrated in vacuo. The residue was dissolved in ethyl acetate and washed with saturated sodium bicarbonate solution and water, dried over sodium sulphate and concentrated. The obtained compound was stirred in IPA, filtered and dried to afford (0.32 g, 18%) 186 as an off-white solid.

2-(3-Chloro-4-(5-(8-chloro-6-(trifluoromethyl)imidazo[1,2-a]pyridin-2-yl)-1,2,4-oxadiazol-3-yl)phenyl)ethanol (187). To a stirred solution of 186 (7 g, 14.85 mmol) in dry DCM (70 mL) was added DIBAL (10.564 g, 74.27 mmol) dropwise at −78° C. and stirred for 2 h. The reaction mixture was quenched with NH₄Cl at −40° C. and extracted with ethyl acetate. The organic layer was washed with water and brine, dried over sodium sulphate, concentrated and chromatographed (EtOAc/Hexane, 1:4) to afford 187 as a pale white solid (2.0 g, 30% yield).

2-(3-Chloro-4-(5-(8-chloro-6-(trifluoromethyl)imidazo[1,2-a]pyridin-2-yl)-1,2,4-oxadiazol-3-yl)phenyl)acetaldehyde (188). To a stirred solution of 187 (1.0 g, 2.25 mmol) in DCM (10 mL) was added DMP (1.148 g, 2.70 mmol) and the reaction mixture was stirred at rt for 12 hours. The reaction mixture was basified with a 1:1 aq solution of sodium thiosulphate and sodium bicarbonate, and the reaction mixture was extracted with DCM. The organic layer was dried over MgSO₄, filtered, concentrated. The solid compound was washed with isopropanol to give 188 (0.8 g, 81% yield).

2-Amino-3-(3-chloro-4-(5-(8-chloro-6-(trifluoromethyl)imidazo[1,2-a]pyridin-2-yl)-1,2,4-oxadiazol-3-yl)phenyl)propanenitrile (189). To a stirred suspension of 188 (0.8 g, 1.81 mmol), NH₄OH (0.2 mL), in MeOH (10 mL) was added AcOH to adjust pH to 4-5. NaCN (0.177 g, 3.4 mmol) was then added and reaction mixture was stirred at rt 16 h. Solvent was removed and water was added to reaction mixture and extracted with DCM. The organic layer was dried over MgSO₄, filtered, concentrated and chromatographed (MeOH:CHCl₃, 1:19) to obtain 189 as a yellow foam (0.2 g, 24% yield).

3-Chloro-4-{5-[8-chloro-6-(trifluoromethyl)imidazo[1,2-a]pyridin-2-yl]-1,2,4-oxadiazol-3-yl}phenylalanine. A solution of 189 (0.1 g, 0.21 mmol) in conc HCl (3 mL) was refluxed for 16 h. Water was removed under reduced pressure and the salt obtained was washed with diethyl ether to obtain 3-chloro-4-{5-[8-chloro-6-(trifluoromethyl)imidazo[1,2-a]pyridin-2-yl]-1,2,4-oxadiazol-3-yl}phenylalanine as off white solid (0.015 g, 14% yield. ¹H NMR (400 MHz, DMSO-d₆) δ 9.4 (s, 1H), 9.0 (s, 1H), 8.4 (s, 2H), 8.1 (s, 1H), 8.0 (d, 1H), 7.7 (s, 1H), 7.5 (d, 1H), 4.4 (s, 1H), 3.3 (s, 2H); MS (EI) for C₁₉H₁₂C₁₂F₃N₅O₃, found 486 (MH+).

### Example 56

### 2-(3-Chloro-4-{5-8-chloro-6-(trifluoromethyl)imidazo[1,2-a]pyridin-2-yl]-1,2,4-oxadiazol-3-yl}phenyl)propan-2-ol

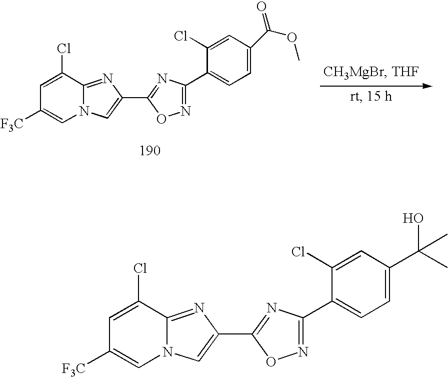

2-(3-Chloro-4-{5-[8-chloro-6-(trifluoromethyl)imidazo[1,2-a]pyridin-2-yl]-1,2,4-oxadiazol-3-yl}phenyl)propan-2-ol. Compound 190 was synthesized in the same manner as compound 41 in Example 6, substituting with appropriate reagents. To a stirred solution of 190 (0.208 g, 0.456 mmol) in THF (15 mL) was added a THF solution of MeMgBr (0.912 mL, 0.325 g, 2.77 mmol) at 0° C. dropwise and the reaction mixture was stirred at rt for another 15 h. Ammonium chloride solution was added to the reaction mixture and the aq layer was extracted with EtOAc. The combined organic fractions were washed with water and brine, dried over Na₂SO₄, concentrated and the crude mass obtained was purified by prep HPLC to obtain 2-(3-chloro-4-{5-[8-chloro-6-(trifluoromethyl)imidazo[1,2-a]pyridin-2-yl]-1,2,4-oxadiazol-3-yl}phenyl)propan-2-ol (0.07 g, 34% yield). ¹H NMR (400 MHz, CDCl₃,) δ 8.6 (s, 2H), 8.1 (d, 1H), 7.7 (s, 1H), 7.5 (m, 2H), 1.7 (s, 3H), 1.5 (s, 3H); MS (EI) for C₁₉H₁₃Cl₂F₃N₄O₂, found 457 (MH+).

### Example 57

### (3-Chloro-4-{5-[8-chloro-6-(trifluoromethyl)imidazo[1,2-a]pyridin-2-yl]-1,2,4-oxadiazol-3-yl}phenyl)acetic acid

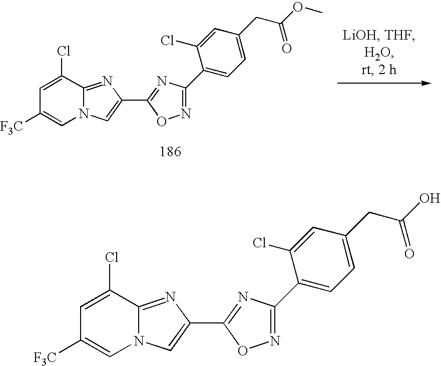

(3-Chloro-4-{5-[8-chloro-6-(trifluoromethyl)imidazo[1,2-a]pyridin-2-yl]-1,2,4-oxadiazol-3-yl}phenyl)acetic acid. To a stirred solution of 186 (0.30 g, 0.64 mmol), synthesized as outlined in Example 55, in THF:Water (10 mL, 1:1) was added LiOH.H₂O (0.067 g, 1.6 mmol) and the stirring was continued for 2 h at rt. Solvent was removed and acetic acid was added at 0° C. to obtain pH 2.5. The mixture was stirred at rt for 30 min, the resulting solids were filtered and washed with water. Solid obtained was crystallized from isopropanol to give (3-chloro-4-{5-[8-chloro-6-(trifluoromethyl)imidazo[1,2-a]pyridin-2-yl]-1,2,4-oxadiazol-3-yl}phenyl)acetic acid as an off-white solid (0.08 g, 27.5% yield). ¹H NMR (400 MHz, CDCl₃) δ 8.6 (m, 2H), 8.1 (m, 1H), 7.5 (m, 2H), 7.3 (m, 1H), 3.7 (s, 2H); MS (EI) for C₁₈H₉Cl₂F₃N₄O₃, found 457 (MH+).

### Example 58

### 4-Amino-3-(3-chloro-4-{5-[8-chloro-6-(trifluoromethyl)imidazo[1,2-a]pyridin-2-yl]-1,2,4-oxadiazol-3-yl}phenyl)-4-oxobutanoic acid

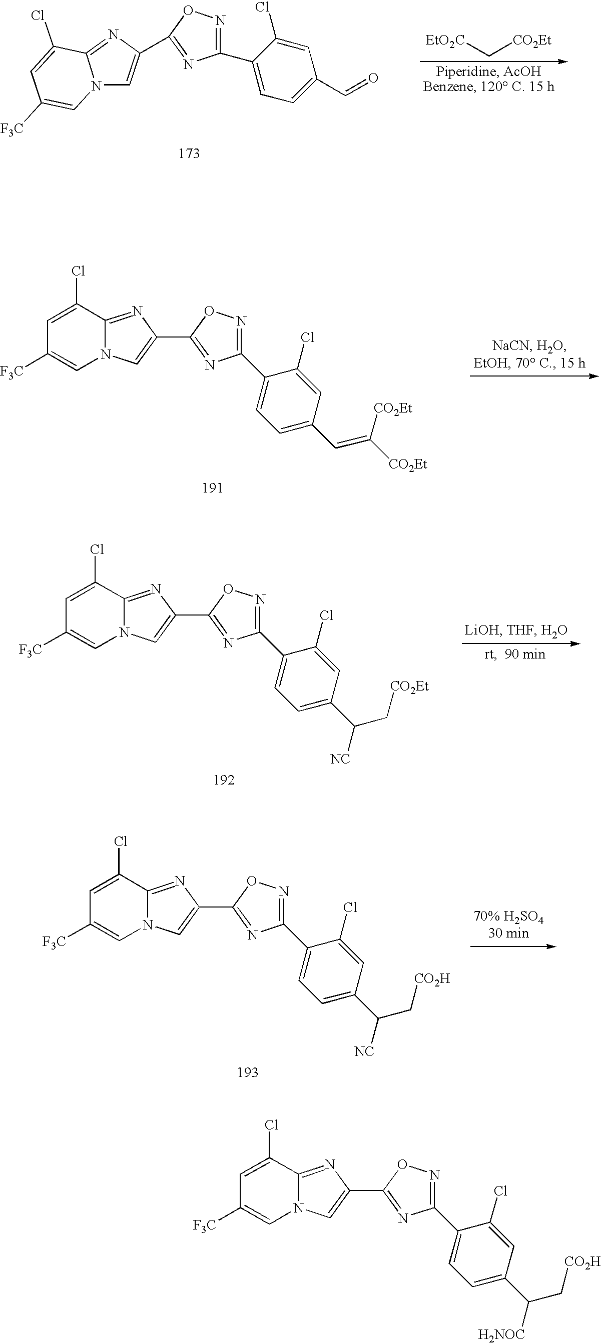

Diethyl 2-(3-chloro-4-(5-(8-chloro-6-(trifluoromethyl)imidazo[1,2-a]pyridin-2-yl)-1,2,4-oxadiazol-3-yl)benzylidene)malonate (191). Aldehyde 173 was synthesized in the same manner as intermediate 40 in Example 5, substituting with appropriate reagents. To a stirred solution of 173 (3.5 g, 8.2 mmol) in benzene (40 mL) were added diethyl malonate (2.4 g, 15 mmol), piperidine (0.188 g, 2.3 mmol), and acetic acid (0.245 g, 4.09 mmol). The reaction mixture was then heated at 120° C. for 15 h. Solvent was removed under reduced pressure and residue was extracted with DCM followed by washing with Aq NaHCO₃. The organic extract was dried over Na₂SO₄, filtered and concentrated. The crude mass was purified by column chromatography (EtOAc/Hexane 3:7) giving of pure compound 191 (3.7 g, 79% yield).

Ethyl 3-(3-chloro-4-(5-(8-chloro-6-(trifluoromethyl)imidazo[1,2-a]pyridin-2-yl)-1,2,4-oxadiazol-3-yl)phenyl)-3-cyanopropanoate (192). To a stirred solution of compound 191 (3.7 g, 6.5 mmol) in ethanol (40 mL) was added water (1.5 mL). NaCN (0.352 g, 7.18 mmol) was added to it portionwise, and the stirring was continued for 40 h at rt. After completion, ethanol was removed under reduced pressure and the residue was extracted with ethyl acetate. The organic extracts were dried over Na₂SO₄, filtered and concentrated. The crude mass was purified by column chromatography (EtOAc/Hexane 1:1) to give 1 g of pure compound 192 in 29.41% yield.

3-(3-Chloro-4-{5-[8-chloro-6-(trifluoromethyl)imidazo[1,2-a]pyridin-2-yl]-1,2,4-oxadiazol-3-yl}phenyl)-3-cyanopropanoic acid (193). To a stirred solution of 192 (1.0 g, 1.9 mmol) in THF (12 mL) was added a solution of LiOH (0.246 g, 6 mmol) in water (5 mL) and the stirring was continued for 90 min at rt. The reaction mixture was diluted with 10% citric acid solution and the reaction mixture was extracted with ethyl acetate. The organic extract was dried over Na₂SO₄, filtered and concentrated. The crude mass was purified by column chromatography (EtOAc) giving pure 3-(3-chloro-4-{5-[8-chloro-6-(trifluoromethyl)imidazo[1,2-a]pyridin-2-yl]-1,2,4-oxadiazol-3-yl}phenyl)-3-cyanopropanoic acid (0.38 g, 40.4% yield). ¹H NMR (400 MHz, DMSO-d₆) δ 12.7 (bs, 1H), 9.4 (s, 1H), 9.0 (s, 1H), 8.1 (m, 1H), 7.9 (s, 1H), 7.7 (d, 1H), 4.7 (m, 1H), 3.2 (m, 1H), 3.0 (m, 1H); MS (EI) for C₂₀H₁₀Cl₂F₃N₅O₃, found 496 (MH+).

4-Amino-3-(3-chloro-4-{5-[8-chloro-6-(trifluoromethyl)imidazo[1,2-a]pyridin-2-yl]-1,2,4-oxadiazol-3-yl}phenyl)-4-oxobutanoic acid. Compound 193 (0.19 g, 0.38 mmol) was dissolved in 2 mL of 70% H₂SO₄ and stirred for 30 min. Reaction mixture was basified with sodium bicarbonate solution and then acidified with 10% citric acid and the aq. layer was extracted with EtOAc. The organic extract was dried over anhydrous Na₂SO₄, filtered and concentrated. The crude mass was purified by preparative HPLC to give of pure 4-amino-3-(3-chloro-4-{5-[8-chloro-6-(trifluoromethyl)imidazo[1,2-a]pyridin-2-yl]-1,2,4-oxadiazol-3-yl}phenyl)-4-oxobutanoic acid (0.186 g, 94.9% yield). ¹H NMR (400 MHz, DMSO-d₆,) δ 12.3 (bs, 1H), 9.4 (s, 1H), 9.0 (s, 1H), 8.0 (m, 2H), 7.7 (s, 2H), 7.5 (d, 1H), 4.0 (t, 1H), 3.0 (m, 1H), 2.6 (m, 1H); MS (EI) for C₂₀H₁₂Cl₂F₃N₅O₄, found 514 (MH+).

### Example 59

### 3-(3-Chloro-4-{5-[8-chloro-6-(trifluoromethyl)imidazo[1,2-a]pyridin-2-yl]-1,2,4-oxadiazol-3-yl}phenyl)-3-(ethylamino)propanoic acid

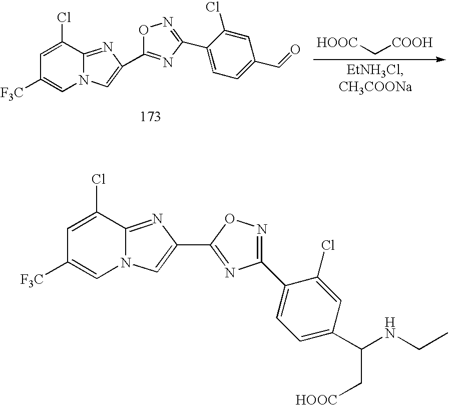

3-(3-Chloro-4-{5-[8-chloro-6-(trifluoromethyl)imidazo[1,2-a]pyridin-2-yl]-1,2,4-oxadiazol-3-yl}phenyl)-3-(ethylamino)propanoic acid. Aldehyde 173 was synthesized in the same manner as intermediate 40 in Example 5, substituting with appropriate reagents. To a stirred solution of CH₃CO₂Na (1.44 g, 17.56 mmol) in ethanol (15 mL) was added C₂H₅NH₃Cl (1.43 g, 17.65 mmol) and stirred at rt for 40 min. To this was added 173 (1.5 g, 3.5 mmol) and malonic acid (0.365 g, 3.5 mmol) and reaction mixture was heated to 80° C. for 12 h. A yellowish solid precipitated out which was filtered, washed with EtOH and purified by prep HPLC giving 3-(3-chloro-4-{5-[8-chloro-6-(trifluoromethyl)imidazo[1,2-a]pyridin-2-yl]-1,2,4-oxadiazol-3-yl}phenyl)-3-(ethylamino)propanoic acid as off-white solid (0.09 g, 5% yield). ¹H NMR (400 MHz, DMSO-d₆) δ 12.8 (bs, 1H), 9.4 (s, 1H), 9.1 (s, 1H), 9.0 (s, 1H), 8.2 (m, 2H), 7.9 (s, 1H), 7.7 (d, 1H), 4.7 (s, 1H), 3.4 (m, 1H), 3.1 (m, 2H), 1.2 (m, 3H); MS (EI) for C₂₁H₁₆Cl₂F₃N₅O₃, found 514 (MH+).

### Example 60

### Amino-3-(3-chloro-4-{5-[8-chloro-6-(trifluoromethyl)imidazo[1,2-a]pyridin-2-yl]-1,2,4-oxadiazol-3-yl}phenyl)propanoic acid

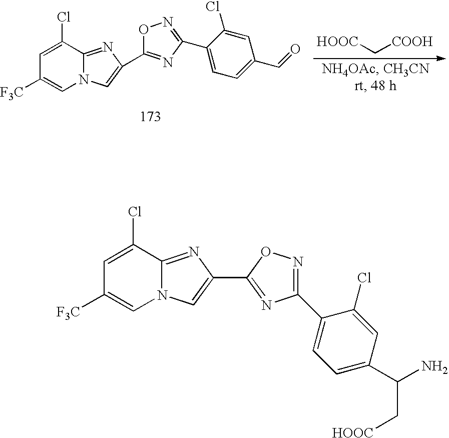

Amino-3-(3-chloro-4-{5-[8-chloro-6-(trifluoromethyl)imidazo[1,2-a]pyridin-2-yl]-1,2,4-oxadiazol-3-yl}phenyl)propanoic acid. Aldehyde 173 was synthesized in the same manner as intermediate 40 in Example 5, substituting with appropriate reagents. A stirred solution of malonic acid (0.071 g, 0.7 mmol), ammonium acetate (0.054 g, 0.7 mmol) and aldehyde 173 (0.15 g, 0.351 mmol) in acetonitrile (8 mL) was stirred for 48 h at rt under argon atmosphere. A white precipitate resulted which was filtered and purified by preparative HPLC to obtain amino-3-(3-chloro-4-{5-[8-chloro-6-(trifluoromethyl)imidazo[1,2-a]pyridin-2-yl]-1,2,4-oxadiazol-3-yl}phenyl)propanoic acid as a white solid (0.035 g, 20.58% yield). ¹H NMR (400 MHz, DMSO-d₆) δ 9.4 (s, 1H), 9.1 (s, 1H), 8.1 (m, 2H), 7.9 (s, 1H), 7.7 (m, 1H), 3.0 (m, 1H), 2.6 (s, 2H); MS (EI) for C₁₉H₁₂Cl₂F₃N₅O₃, found 486 (MH+).

### Example 61

### 2-({2-[(2,5-Dichloro-4-{5-[8-chloro-6-(trifluoromethyl)imidazo[1,2-a]pyridin-2-yl]-1,2,4-oxadiazol-3-yl}phenyl)oxy]ethyl}amino)propane-1,3-diol

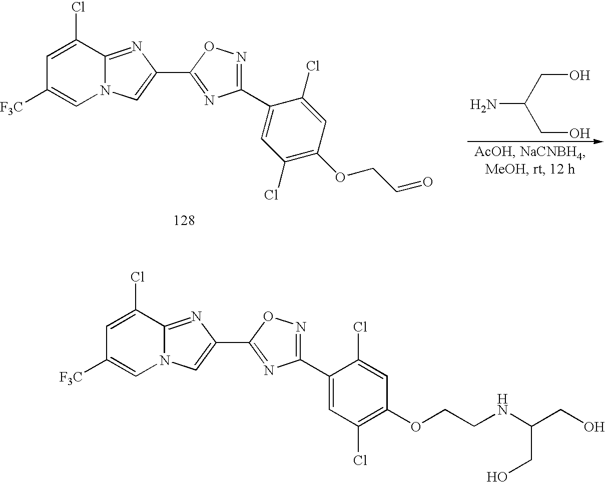

2-[(2-[(2,5-Dichloro-4-{5-[8-chloro-6-(trifluoromethyl)imidazo[1,2-a]pyridin-2-yl]-1,2,4-oxadiazol-3-yl}phenyl)oxy]ethyl}amino)propane-1,3-diol. To a stirred solution of aldehyde 128 (0.575 g, 1.17 mmol), synthesized as outlined in Example 26, in MeOH (10 mL) was added 2-amino-1,3 propanediol (0.118 g, 1.287 mmol) and AcOH (0.5 mL) at rt and the reaction mixture was stirred for 30 min. NaCNBH₄ (0.073 g, 1.17 mmol) was added and the reaction mixture was stirred at rt for 12 h. Solvent was removed and the crude mass was purified by preparative HPLC to give 2-({2-[(2,5-dichloro-4-{5-[8-chloro-6-(trifluoromethyl)imidazo[1,2-a]pyridin-2-yl]-1,2,4-oxadiazol-3-yl}phenyl)oxy]ethyl}amino)propane-1,3-diol as a white solid (0.06 g, 9.09% yield). ¹H NMR (400 MHz, DMSO-d₆) δ 9.3 (s, 1H), 9.1 (s, 1H), 8.7 (s, 1H) 8.2 (s, 1H), 8.1 (s, 1H), 7.6 (s, 1H), 5.4 (bs, 2H), 4.6 (m, 2H), 3.4-3.8 (m, 6H); MS (EI) for C₂₁H₁₇Cl₃F₃N₅O₄, found 566 (MH+).

### Example 62

### (2R)-2-[(2,5-Dichloro-4-{5-[8-chloro-6-(trifluoromethyl)imidazo[1,2-a]pyridin-2-yl]-1,2,4-oxadiazol-3-yl}phenyl)oxy]propan-1-ol

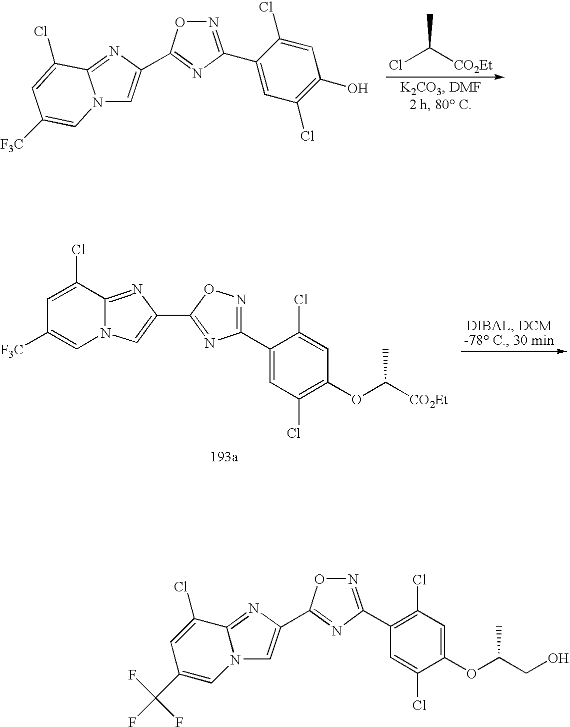

(R)-Ethyl 2-(2,5-dichloro-4-(5-(8-chloro-6-(trifluoromethyl)imidazo[1,2-a]pyridin-2-yl)-1,2,4-oxadiazol-3-yl)phenoxy)propanoate (193a). A stirred solution of 2,5-dichloro-4-(5-(8-chloro-6-(trifluoromethyl)imidazo(1,2-a)pyridine-2-yl)-1,2,4-oxadiazole-3-yl)phenol (0.75 g, 1.67 mmol), prepared as described in Example 13, K₂CO₃ (1.15 g, 8.35 mmol) and methyl (R)-(+)-2-chloro propionate (0.839 mL, 8.35 mmol) in DMF (10 mL) was heated to 80° C. for 2 h. DMF was removed under reduced pressure and the resulting residue was partitioned between ethyl acetate and water. The phases were separated and the organic layer was dried, concentrated and the resulting solid washed with pentane to afford 193 (0.73 g, 80%).

(2R)-2-[(2,5-Dichloro-4-{5-[8-chloro-6-(trifluoromethyl)imidazo[1,2-a]pyridin-2-yl]-1,2,4-oxadiazol-3-yl}phenyl)oxy]propan-1-ol. To a solution of 193a (0.35 g, 0.63 mmol) in DCM (25 mL) at −78° C. was added DIBAL (1M solution in toluene, 2.4 mL) very carefully and the reaction mixture was stirred at the same temperature for 30 min. The reaction mixture was allowed to slowly warm to room temperature and further stirred for 40 min. The reaction mixture was recooled to −78° C., quenched with ethyl acetate followed by saturated NH₄Cl solution and stirred at rt for 1 h. The reaction mixture was extracted with ethyl acetate and the organic layer subsequently washed with water and brine, dried, concentrated and purified by prep HPLC to afford the title compound (0.05 g, 16%). ¹H-NMR (400 MHz, DMSO-d₆) δ 9.3 (s, 1H), 9.0 (s, 1H), 8.1 (s, 1H), 8.0 (s, 1H), 7.6 (s, 1H), 4.8 (m, 1H) 3.6 (m, 2H), 1.3 (d, 3H); MS (EI) for C₁₉H₁₂C₁₃F₃N₄O₃, found 506.9 (MH+).

Using the same or analogous synthetic techniques in Example 62 and substituting with appropriate reagents, (2S)-2-[(2,5-dichloro-4-{5-[8-chloro-6-(trifluoromethyl)imidazo[1,2-a]pyridin-2-yl]-1,2,4-oxadiazol-3-yl}phenyl)oxy]propan-1-ol was prepared. ¹H-NMR (400 MHz, DMSO-d₆) δ 9.3 (s, 1H), 9.0 (s, 1H), 8.1 (s, 1H), 8.0 (s, 1H), 7.6 (s, 1H), 4.8 (m, 1H), 3.6 (m, 2H), 1.3 (d, 3H); MS (EI) for C₁₉H₁₂Cl₃F₃N₄O₃, found 506.8 (MH+). Example 63 1-[(2,5-Dichloro-4-{3-[8-chloro-6-(trifluoromethyl)imidazo[1,2-a]pyridin-2-yl]-1,2,4-oxadiazol-5-yl}phenyl)oxy]propan-2-ol

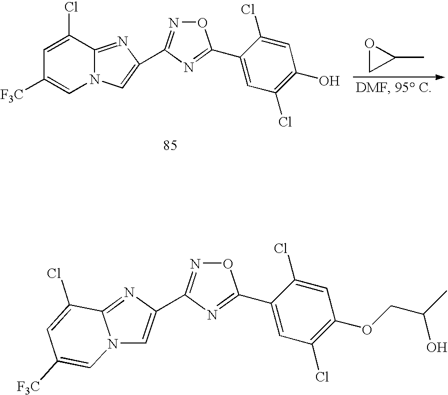

1-[(2,5-Dichloro-4-{3-[8-chloro-6-(trifluoromethyl)imidazo[1,2-a]pyridin-2-yl]-1,2,4-oxadiazol-5-yl}phenyl)oxy]propan-2-ol. A stirring solution of 85 (0.5 g, 1.16 mmol), prepared as described in Example 17, and propylene oxide (4 mL), in DMF (3 mL) was heated in a sealed tube for 48 h at 80° C. Solvent was removed and residue was purified by prep HPLC to obtain the title compound (0.4 g, 71%). ¹H-NMR (400 MHz, DMSO-d₆) δ 9.3 (s, 1H), 8.8 (s, 1H), 8.2 (d, 1H), 8.0 (s, 1H), 7.6 (d, 1H), 4.8 (bs, 1H), 4.1 (m, 2H), 3.6 (d, 1H), 1.2 (d, 3H); MS (EI) for C₁₉H₁₂C₁₃F₃N₄O₃, found 506.9 (MH+).

Using the same or analogous synthetic techniques in Example 63 and substituting with appropriate reagents (prepared using procedures as described herein), 1-[(5-chloro-4-{3-[8-chloro-6-(trifluoromethyl)imidazo[1,2-a]pyridin-2-yl]-1,2,4-oxadiazol-5-yl}-2-fluorophenyl)oxy]propan-2-ol was prepared. ¹H-NMR (400 MHz, DMSO-d₆) δ 9.3 (s, 1H), 8.9 (s, 1H), 8.1 (d, 1H), 8.0 (s, 1H), 7.6 (d, 1H), 4.0 (m, 4H), 1.2 (d, 3H); MS (EI) for C₁₉H₁₂Cl₂F₄N₄O₃, found 491.0 (MH+).

### Example 64

### 2-Amino-3-[(2,5-dichloro-4-{3-[8-chloro-6-(trifluoromethyl)imidazo[1,2-a]pyridin-2-yl]-1,2,4-oxadiazol-5-yl}phenyl)oxy]propan-1-ol

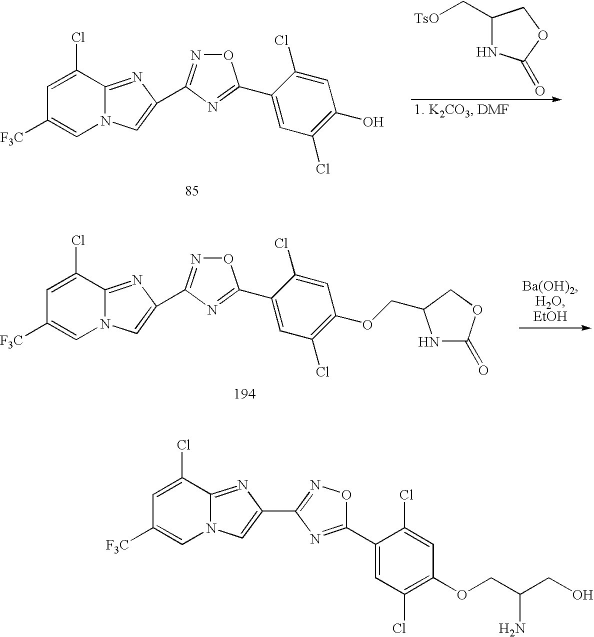

4-((2,5-Dichloro-4-(3-(8-chloro-6-(trifluoromethyl)imidazo[1,2-a]pyridin-2-yl)-1,2,4-oxadiazol-5-yl)phenoxy)methyl)oxazolidin-2-one (194). To a solution of 85 (1.55 g, 3.47 mmol), prepared as described in Example 17, in DMF (15 mL) was added K₂CO₃ (0.96 g, 6.95 mmol) and the reaction mixture was stirred for 15 min at rt, followed by the addition of (2-oxooxazolidin-4-yl)methyl 4-methylbenzenesulfonate (1.5 g, 5.5 mmol). The reaction mixture was heated to 80° C. for 3 h. The reaction was quenched with water (50 mL) and filtered. The resulting solids were washed with acetone and diethyl ether to afford 194 (1.2 g, 67%).

2-Amino-3-[(2,5-dichloro-4-{3-[8-chloro-6-(trifluoromethyl)imidazo[1,2-a]pyridin-2-yl]-1,2,4-oxadiazol-5-yl}phenyl)oxy]propan-1-ol. A stirred solution of 194 (0.4 g, 0.72 mmol) and Ba(OH)₂ (0.45 g, 1.4 mmol) in ethanol (15 mL) and water (7 mL) was heated at 65° C. for 2 h. After cooling to room temperature the reaction mixture was poured into ice water. The resulting solid was filtered, washed with water and stirred in ethanolic HCl (5 mL) for 30 min. Solvent was removed and the crude product obtained was purified by prep HPLC to yield the title compound (0.04 g, 10%). ¹H-NMR (400 MHz, DMSO-d₆) δ 9.3 (s, 1H), 8.8 (s, 1H), 8.2 (s, 1H), 8.0 (s, 1H), 7.1 (s, 1H), 5.9 (d, 1H), 4.9 (s, 2H), 3.8 (s, 3H); MS (EI) for C₁₉H₁₃C₁₃F₃N₅O₃, found 521.8 (MH+).

Using the same or analogous synthetic techniques in Example 64 and substituting with appropriate reagents (prepared using procedures as described herein), 2-amino-3-[(5-chloro-4-{3-[8-chloro-6-(trifluoromethyl)imidazo[1,2-a]pyridin-2-yl]-1,2,4-oxadiazol-5-yl}-2-fluorophenyl)oxy]propan-1-ol was prepared. ¹H-NMR (400 MHz, DMSO-d₆) δ 9.3 (s, 1H), 8.8 (s, 1H), 8.0 (s, 1H), 7.9 (m, 1H), 7.1 (d, 1H), 6.2 (d, 1H), 4.8 (s, 2H), 3.5 (m, 3H); MS (EI) for C₁₉H₁₃Cl₂F₄N₅O₃, found 505.8 (MH+).

### Example 65

### 1-[(2,5-Dichloro-4-{5-[8-chloro-6-(trifluoromethyl)imidazo[1,2-a]pyridin-2-yl]-1,3,4-oxadiazol-2-yl}phenyl)oxy]propan-2-ol

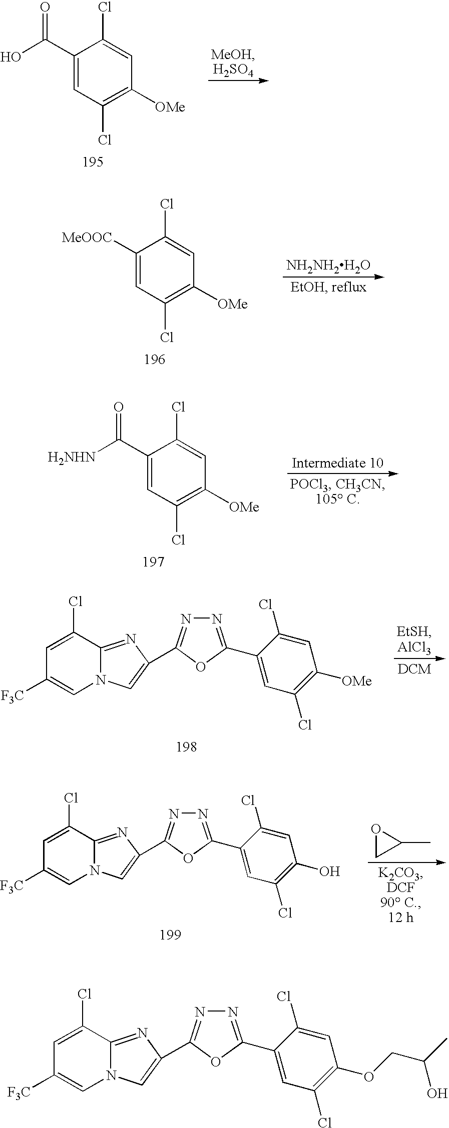

Methyl 2,5-dichloro-4-methoxybenzoate (196). To an ice cold solution of 195 (5.4 g, 24.5 mmol) in ethanol (25 mL) was added H₂SO₄ (5 mL) and the reaction mixture was then refluxed for 3 h. Solvent was removed and the resulting residue was dissolved in EtOAc and washed with NaHCO₃ solution, water, brine, dried and concentrated to afford 196 (5 g, 87%) which was used as is without further purification.

2,5-Dichloro-4-methoxybenzohydrazide (197). A solution of 196 (5.0 g, 21.5 mmol) and NH₂NH₂.H₂O (5.2 mL, 107 mmol) in ethanol (25 mL) was refluxed for 3 h. Solvent was removed and the residue was dissolved in EtOAc and washed with water, brine, dried and concentrated to afford 197 (4.5 g, 80%) which was used as is without further purification.

2-(8-Chloro-6-(trifluoromethyl)imidazo[1,2-a]pyridin-2-yl)-5-(2,5-dichloro-4-methoxyphenyl)-1,3,4-oxadiazole (198). To a stirring solution of 10 (1.73 g, 6.6 mmol) and 197 (1.54 g, 6.6 mmol) in CH₃CN (20 mL) was added POCl₃ (5 mL) and the mixture heated to 105° C. Solvent was removed and the resulting residue was partitioned between NaHCO₃ solution and EtOAc. The organic phase was dried and concentrated to obtain 198 (1.5 g, 49%) which was used as is without further purification.

2,5-Dichloro-4-(5-(8-chloro-6-(trifluoromethyl)imidazo[1,2-a]pyridin-2-yl)-1,3,4-oxadiazol-2-yl)phenol (199). To a cold solution (ice water bath) of 198 (0.250 g, 0.53 mmol) in DCM (7 mL) was added AlCl₃ (0.369 g, 2.7 mmol) in portions under an atmosphere of argon maintaining the temperature below 10° C. The light brown suspension was stirred for 1 h and then EtSH (0.168 g, 2.7 mmol) was added dropwise at 0° C., and stirred for 10 min. at 0° C. and then at rt for 15 h. The reaction mixture was cooled to 0° C. and ice cold water was added. The resulting precipitate was filtered and washed with cold water and cool acetone giving 199 (0.170 g, 70.24%).

1-[(2,5-Dichloro-4-{5-[8-chloro-6-(trifluoromethyl)imidazo[1,2-a]pyridin-2-yl]-1,3,4-oxadiazol-2-yl}phenyl)oxy]propan-2-ol. A stirring mixture of 199 (0.8 g, 1.6 mmol) and propylene oxide (7 mL) in DMF (6 mL) was heated to 70° C. for 2 h, then at 80° C. for 4 h and at 90° C. overnight. Solvent was removed, and the resulting residue was purified by prep HPLC to obtain the title compound (0.06 g, 7%). ¹H-NMR (400 MHz, DMSO-d₆) δ 9.3 (s, 1H), 9.0 (s, 1H), 8.2 (d, 1H), 8.0 (s, 1H), 7.6 (d, 1H), 4.8 (s, 1H), 4.1 (m, 2H), 3.6 (s, 1H), 1.2 (d, 3H)); MS (EI) for C₁₉H₁₂Cl₃F₃N₄O₃, found 506.8 (MH+).

Using the same or analogous synthetic techniques in Example 65 and substituting with appropriate reagents (prepared using procedures as described herein), 1-[(5-chloro-4-{5-[8-chloro-6-(trifluoromethyl)imidazo[1,2-a]pyridin-2-yl]-1,3,4-oxadiazol-2-yl}-2-fluorophenyl)oxy]propan-2-ol was prepared. ¹H-NMR (400 MHz, DMSO-d₆) δ 9.3 (s, 1H), 9.0 (s, 1H), 8.0 (m, 2H), 7.6 (d, 1H), 5.0 (s, 1H), 4.1 (m, 3H), 1.2 (d, 3H); MS (EI) for C₁₉H₁₂Cl₂ F₄N₄O₃, found 491 (MH+).

### Example 66

### 2-Amino-3-[(2,5-dichloro-4-{5-[8-chloro-6-(trifluoromethyl)imidazo[1,2-a]pyridin-2-yl]-1,3,4-oxadiazol-2-yl}phenyl)oxy]propan-1-ol

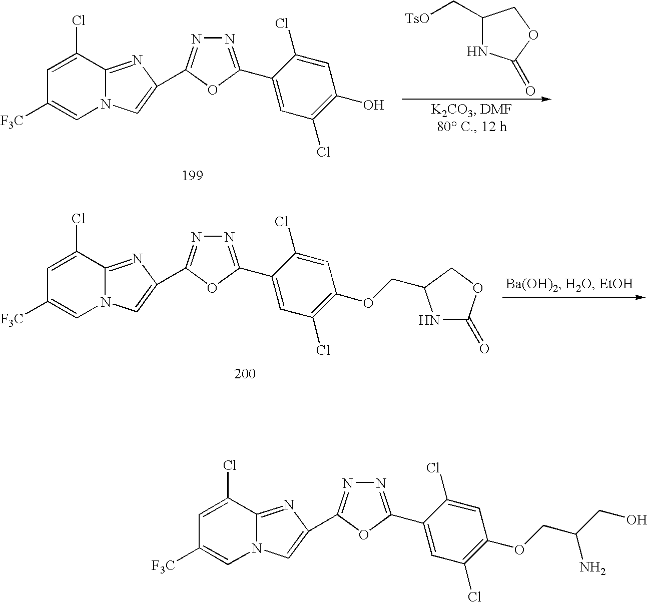

4-((2,5-Dichloro-4-(5-(8-chloro-6-(trifluoromethyl)imidazo[1,2-a]pyridin-2-yl)-1,3,4-oxadiazol-2-yl)phenoxy)methyl)oxazolidin-2-one (200). A stirred suspension of 199 (0.35 g, 0.78 mmol), prepared as described in Example 65, (2-oxooxazolidin-4-yl)methyl 4-methylbenzenesulfonate (0.5 g, 1.8 mmol) and K₂CO₃ (0.44 g, 3.1 mmol) in DMF (7 mL) was heated at 85° C. for 14 h. The reaction mixture was cooled and poured into ice water. The resulting precipitate was filtered and washed with water, hexanes and dried to obtain 200 (0.35 g, 82%) which was used in subsequent reactions without further purification.

2-Amino-3-[(2,5-dichloro-4-{5-[8-chloro-6-(trifluoromethyl)imidazo[1,2-a]pyridin-2-yl]-1,3,4-oxadiazol-2-yl}phenyl)oxy]propan-1-ol. A stirred solution of 200 (0.35 g, 0.69 mmol) and Ba(OH)₂ (0.45 g, 1.8 mmol) in ethanol (6 mL) and water (12 mL) was heated at 65° C. for 2 h. The reaction mixture was then cooled and poured into ice water. The resulting precipitate was filtered and washed with water, IPA, dried and then purified by prep HPLC to obtain the title compound (0.02 g, 6%). ¹H-NMR (400 MHz, DMSO-d₆) δ 9.3 (s, 1H), 9.0 (s, 1H), 8.2 (s, 1H), 8.1 (s, 2H), 8.0 (s, 1H), 7.6 (s, 1H), 5.4 (s, 1H), 4.4 (m, 2H), 3.7 (m, 3H); MS (EI) for C₁₉H₁₃Cl₃F₃N₅O₃, found 521.9 (MH+).

Using the same or analogous synthetic techniques in Example 66 and substituting with appropriate reagents (prepared using procedures as described herein), 2-amino-3-[(5-chloro-4-{5-[8-chloro-6-(trifluoromethyl)imidazo[1,2-a]pyridin-2-yl]-1,3,4-oxadiazol-2-yl}-2-fluorophenyl)oxy]propan-1-ol was prepared. ¹H-NMR (400 MHz, DMSO-d₆) δ 9.3 (s, 1H), 9.0 (s, 1H), 8.1 (s, 2H), 8.0 (m, 2H), 7.6 (d, 1H), 5.4 (t, 1H), 4.4 (m, 2H), 3.7 (m, 3H); MS (EI) for C₁₉H₁₃Cl₂F₄N₅O₃, found 505.9 (MH+).

### Example 67

### 1-[(2,5-Dichloro-4-{5-[8-chloro-6-(trifluoromethyl)imidazo[1,2-a]pyridin-2-yl]-1,3,4-thiadiazol-2-yl}phenyl)oxy]propan-2-ol

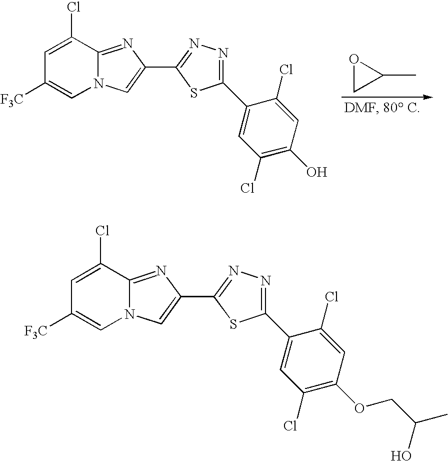

1-[(2,5-Dichloro-4-{5-[8-chloro-6-(trifluoromethyl)imidazo[1,2-a]pyridin-2-yl]-1,3,4-thiadiazol-2-yl}phenyl)oxy]propan-2-ol. A stirring solution of 2,5-dichloro-4-(5-(8-chloro-6-(trifluoromethyl)imidazo[1,2-a]pyridin-2-yl)-1,3,4-thiadiazol-2-yl)phenol (0.5 g, 1.16 mmol), prepared as described in Example 18, and propylene oxide (4 mL) in DMF (3 mL) was heated in sealed tube for 48 h at 80° C. Solvent was removed and residue was purified by prep HPLC to obtain the title compound (0.07 g, 12%). ¹H-NMR (400 MHz, DMSO-d₆) δ 9.3 (s, 1H), 8.8 (s, 1H), 8.3 (s, 1H), 8.0 (s, 1H), 7.5 (s, 1H), 5.0 (s, 1H), 4.0 (m, 2H), 3.6 (m, 1H), 1.2 (d, 3H); MS (EI) for C₁₉H₁₂Cl₃F₃N₄O₂S, found 523 (MH+).

### Example 68

### (2S)-2-Amino-3-[(2,5-dichloro-4-{5-[8-chloro-6-(trifluoromethyl)imidazo[1,2-a]pyridin-2-yl]-1,3,4-thiadiazol-2-yl}phenyl)oxy]propan-1-ol

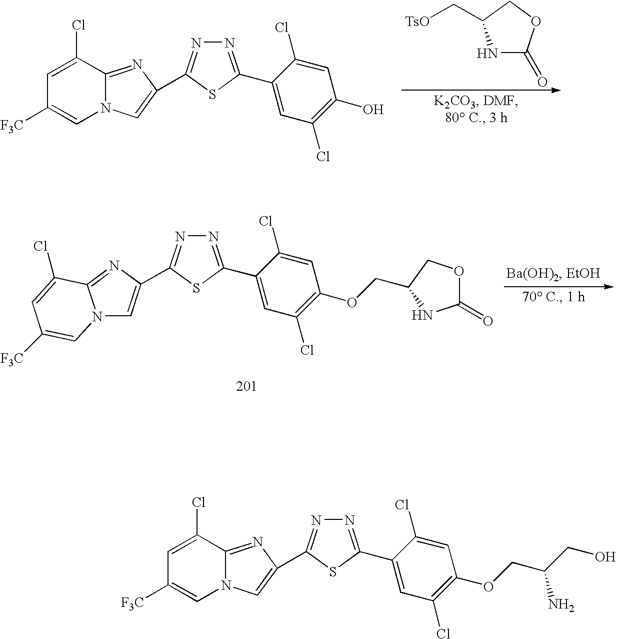

(R)-4-((2,5-Dichloro-4-(5-(8-chloro-6-(trifluoromethyl)imidazo[1,2-a]pyridin-2-yl)-1,3,4-thiadiazol-2-yl)phenoxy)methyl)oxazolidin-2-one (201). To a solution of 2,5-dichloro-4-(5-(8-chloro-6-(trifluoromethyl)imidazo[1,2-a]pyridin-2-yl)-1,3,4-thiadiazol-2-yl)phenol (1.5 g, 3.47 mmol), prepared as described in Example 18, in DMF (15 mL) was added K₂CO₃ (0.96 g, 6.95 mmol) and the reaction mixture was stirred for 15 min at rt followed by the addition of (2-oxooxazolidin-4-yl)methyl 4-methylbenzenesulfonate (1.5 g, 5.5 mmol). The resulting reaction mixture was heated to 80° C. for 3 h. The reaction was quenched with water (50 mL) and the resulting solids filtered and washed with acetone and diethyl ether to afford 201 (1.2 g, 66%).

(2S)-2-Amino-3-[(2,5-dichloro-4-{5-[8-chloro-6-(trifluoromethyl)imidazo[1,2-a]pyridin-2-yl]-1,3,4-thiadiazol-2-yl}phenyl)oxy]propan-1-ol. A stirred solution of 201 (0.83 g, 1.4 mmol) and Ba(OH)₂ (1.0 g, 5.8 mmol) in ethanol (15 mL) and water (10 mL) was heated at 70° C. for 1 h. The reaction mixture was cooled and poured into ice water. The resulting precipitate was filtered and washed with water, IPA, dried and then purified by prep HPLC to obtain the title compound (0.05 g, 6%). ¹H-NMR (400 MHz, DMSO-d₆) δ 9.3 (s, 1H), 8.8 (s, 1H), 8.3 (s, 1H), 8.2 (s, 2H), 8.0 (s, 1H), 7.6 (s, 1H), 5.4 (s, 1H), 4.4 (m, 2H), 3.7 (m, 3H); MS (EI) for C₁₉H₁₃C₁₃F₃N₅O₂S, found 538.0 (MH+).

The following compounds were prepared using the same or analogous synthetic techniques in Example 68 and substituting with appropriate reagents, which were commercially available or were prepared using procedures herein or procedures known to one of ordinary skill in the art.

2-Amino-3-[(2,5-dichloro-4-{5-[8-chloro-6-(trifluoromethyl)imidazo[1,2-a]pyridin-2-yl]-1,3,4-thiadiazol-2-yl}phenyl)oxy]propan-1-ol. ¹H-NMR (400 MHz, DMSO-d₆) δ 9.3 (s, 1H), 8.8 (s, 1H), 8.3 (s, 1H), 8.2 (s, 2H), 8.0 (s, 1H), 7.6 (s, 1H), 5.4 (s, 1H), 4.4 (m, 2H), 3.7 (m, 3H); MS (EI) for C₁₉H₁₃Cl₃F₃N₅O₂S, found 537.9 (MH+). 2-Amino-3-({5-chloro-4-[5-(8-chloroimidazo[1,2-a]pyridin-2-yl)-1,3,4-thiadiazol-2-yl]-2-fluorophenyl}oxy)propan-1-ol. ¹H-NMR (400 MHz, DMSO-d₆) δ 8.9 (s, 1H), 8.7 (d, 1H), 8.4 (s, 2H), 8.2 (d, 1H), 7.7 (d, 1H), 7.6 (d, 1H), 7.0 (t, 1H), 4.4 (m, 3H), 3.8 (m, 2H), 3.6 (s, 1H); MS (EI) for C₁₈H₁₄Cl₂FN₅O₂S, found 453.9 (MH+).

2-Amino-3-[(5-chloro-2-fluoro-4-{5-[6-(trifluoromethyl)imidazo[1,2-a]pyridin-2-yl]-1,3,4-thiadiazol-2-yl}phenyl)oxy]propan-1-ol. ¹H-NMR (400 MHz, DMSO-d₆) δ 9.34 (s, 1H), 8.84 (s, 1H), 8.16 (d, 1H), 7.90 (d, 1H), 7.65 (d, 1H), 7.60 (d, 1H), 4.74 (m, 1H), 4.10 (m, 2H), 3.43 (m, 2H), 3.08 (m, 1H). MS (EI) for C₁₉H₁₄ClF₄N₅O₂S, found 488.1 (MH+). 2-Amino-3-({4-[5-(6-bromoimidazo[1,2-a]pyridin-2-yl)-1,3,4-thiadiazol-2-yl]-5-chloro-2-fluorophenyl}oxy)propan-1-ol. ¹H-NMR (400 MHz, DMSO-d₆) δ 9.0 (s, 1H), 8.7 (s, 1H), 8.2-8.3 (m, 3H), 7.7 (m, 2H), 7.5 (d, 1H), 5.4 (s, 1H), 4.4 (m, 2H), 3.5-3.8 (m, 3H); MS (EI) for C₁₈H₁₄BrCl_{F}N₅O₂S, found 499.8 (MH+).

2-Amino-3-[(2,5-dichloro-4-{5-[6-(trifluoromethyl)imidazo[1,2-a]pyridin-2-yl]-1,3,4-thiadiazol-2-yl}phenyl)oxy]propan-1-ol. ¹H-NMR (400 MHz, DMSO-d₆) δ 9.3 (s, 1H), 8.8 (s, 1H), 8.4 (s, 1H), 8.25 (bs, 2H), 7.9 (d, 1H), 7.6-7.7 (m, 2H), 5.5(t, 1H), 4.4 (m, 2H), 3.75 (m, 2H), 3.6(m, 1H); MS (EI) for C₁₉H₁₄Cl₂F₃N₅O₂S, found 503.8 (MH+). 2-Amino-3-({5-chloro-4-[5-(6-chloroimidazo[1,2-a]pyridin-2-yl)-1,3,4-thiadiazol-2-yl]-2-fluorophenyl}oxy)propan-1-ol. ¹H-NMR (400 MHz, DMSO-d₆) δ 9.0 (s, 1H), 8.7 (s, 1H), 8.2 (m, 3H), 7.7 (d, 1H), 7.6 (d, 1H), 7.5 (d, 1H), 5.4 (bs, 1H), 4.4 (m, 2H), 3.5-3.8 (m, 3H); MS (EI) for C₁₈H₁₄Cl₂FN₅O₂S, found 453.5 (MH+). 2-Amino-3-({5-chloro-2-fluoro-4-[5-(6-iodoimidazo[1,2-a]pyridin-2-yl)-1,3,4-thiadiazol-2-yl]phenyl}oxy)propan-1-ol. ¹H-NMR (400 MHz, DMSO-d₆) δ 9.0 (s, 1H), 8.6 (s, 1H), 8.2 (m, 3H), 7.6 (d, 1H), 7.5 (m, 2H), 5.4 (bs, 1H), 4.4 (s, 1H), 4.3 (s, 1H), 3.7 (d, 2H), 3.6 (s, 1H); MS (EI) for C₁₈H₁₄ClFIN₅O₂S, found 545.8 (MH+). (2R)-2-Amino-3-[(5-chloro-2-fluoro-4-{5-[6-(trifluoromethyl)imidazo[1,2-a]pyridin-2-yl]-1,3,4-thiadiazol-2-yl}phenyl)oxy]propan-1-ol. ¹H-NMR (400 MHz, TFA) δ 9.3 (s, 1H), 8.8 (s, 1H), 8.2 (m, 4H), 7.9 (d, 1H), 7.8 (d, 1H), 5.4 (bs, 1H), 4.4 (m, 2H), 3.6 (m, 3H); MS (EI) for C₁₉H₁₄ClF₄N₅O₂S, found 488 (MH+). 2-Amino-3-[(5-chloro-2-fluoro-4-{5-[6-(methyloxy)imidazo[1,2-a]pyridin-2-yl]-1,3,4-thiadiazol-2-yl}phenyl)oxy]propan-1-ol. ¹H-NMR (400 MHz, TFA) δ 8.7 (s, 1H), 8.3 (s, 1H), 8.1 (d, 1H), 7.9 (m, 2H), 7.4 (d, 1H), 5.0 (s, 2H), 4.7 (m, 2H), 4.5 (s, 1H), 4.0 (s, 3H); MS (EI) for C₁₉H₁₇ClFN₅O₃S, found 450 (MH+). (2S)-2-Amino-3-[(5-chloro-2-fluoro-4-{5-[6-(trifluoromethyl)imidazo[1,2-a]pyridin-2-yl]-1,3,4-thiadiazol-2-yl}phenyl)oxy]propan-1-ol. ¹H-NMR (400 MHz, DMSO-d₆) δ 9.4 (s, 1H), 8.8 (s, 1H), 8.3 (s, 2H), 8.2 (d, 1H), 7.9 (d, 1H), 7.6 (t, 2H), 5.4 (s, 1H), 4.4 (m, 2H), 3.7 (m, 2H), 3.6 (s, 1H); MS (EI) for C₁₉H₁₄ClF₄N₅O₂S, found 487.9 (MH+). (2S)-2-Amino-3-[(5-chloro-2-fluoro-4-{5-[6-(methyloxy)imidazo[1,2-a]pyridin-2-yl]-1,3,4-thiadiazol-2-yl}phenyl)oxy]propan-1-ol. ¹H-NMR (400 MHz, DMSO-d₆) δ 8.60 (s, 1H), 8.35 (s, 1H), 8.20 (br s, 2H), 8.15 (d, 1H), 7.60 (m, 2H), 7.15 (m, 1H), 4.35 (m, 2H), 3.80 (s, 3H), 3.70 (m, 2H), 3.55 (m, 1H); MS (EI) for C₁₉H₁₇ClFN₅O₃S, found 450 (MH+). Example 69 2-Amino-3-[(5-chloro-4-{5-[8-chloro-6-(trifluoromethyl)imidazo[1,2-a]pyridin-2-yl]-1,3,4-thiadiazol-2yl}-2-fluorophenyl)oxy]propyl dihydrogen phosphate

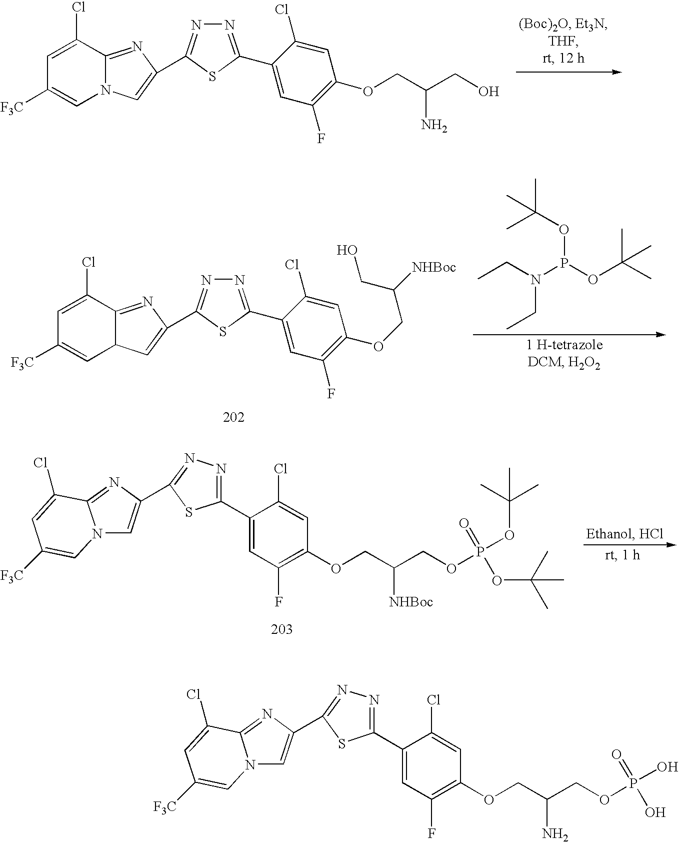

tert-Butyl 1-(5-chloro-4-(5-(7-chloro-5-(trifluoromethyl)-3aH-indol-2-yl)-1,3,4-thiadiazol-2-yl)-2-fluorophenoxy)-3-hydroxypropan-2-ylcarbamate (202). To a stirred solution of 2-amino-3-[(5-chloro-4-{5-[8-chloro-6-(trifluoromethyl)imidazo[1,2-a]pyridin-2-yl]-1,3,4-thiadiazol-2yl}-2-fluorophenyl)oxy]propan-1-ol (1.0, 1.9 mmol), prepared as described in Example 47, and Et₃N (0.5 mL, 3.5 mmol) in THF (20 mL) was added Boc anhydride (0.586 g, 2.69 mmol) at rt and the reaction mixture was stirred for 12 h. Solvent was then removed and water was added to the reaction mixture. The resulting solid was filtered and washed with ether to obtain of 202 as a white solid (1.1 g, 93%).

tert-Butyl 1-(5-chloro-4-(5-(8-chloro-6-(trifluoromethyl)imidazo[1,2-a]pyridin-2-yl)-1,3,4-thiadiazol-2-yl)-2-fluorophenoxy)-3-(di-tert-butoxyphosphoryloxy)propan-2-ylcarbamate (203). To a ice cooled solution of 202 (1.1 g, 1.77 mmol) in DCM (10 mL) was added di-tert-butyl diethylphosphoramidite (1.23 mL, 4.4 mmol) followed by tetrazole (9.84 mL, 1 M solution in CH₃CN) and the reaction mixture was stirred at rt for 3 h. Hydrogen peroxide (30 mL, 30%) was added to the reaction mixture at 0° C. and stirring was continued for 30 min at 0° C. A saturated solution of sodium thiosulphate (40 mL) was then added dropwise and the reaction mixture was stirred at the same temp for 2 h. The resulting solids were filtered from the reaction mixture and washed with water and dried by azeotropic distillation giving 203 (0.75 g, 54%).

2-Amino-3-[(5-chloro-4-{5-[8-chloro-6-(trifluoromethyl)imidazo[1,2-a]pyridin-2-yl]-1,3,4-thiadiazol-2-yl}-2-fluorophenyl)oxy]propyl dihydrogen phosphate. To an ice cold solution of 203 (0.75 g, 0.9 mmol)) in ethanol (5 mL) was added ethanolic HCl (20 mL), and the reaction mixture was stirred at rt for 1 h. Solvent was then removed and solid obtained was washed with ether, DMSO, NMP and ether successively to give the title compound as a white solid (0.12 g, 22%). ¹H-NMR (400 MHz, DMSO-d₆) δ 9.1 (s, 1H), 9.0 (s, 1H), 8.2 (s, 1H), 8.1 (d, 1H), 7.3 (s, 1H), 4.2-5.0 (m, 5H); MS (EI) for C₁₉H₁₄Cl₂F₄N₅O₅PS, found 601.8 (MH+).

### Example 70

### 1-[(5-chloro-4-{5-[8-chloro-6-(trifluoromethyl)imidazo[1,2-a]pyridin-2-yl]-1,3,4-thiadiazol-2-yl}-2-fluorophenyl)oxy]propan-2-amine

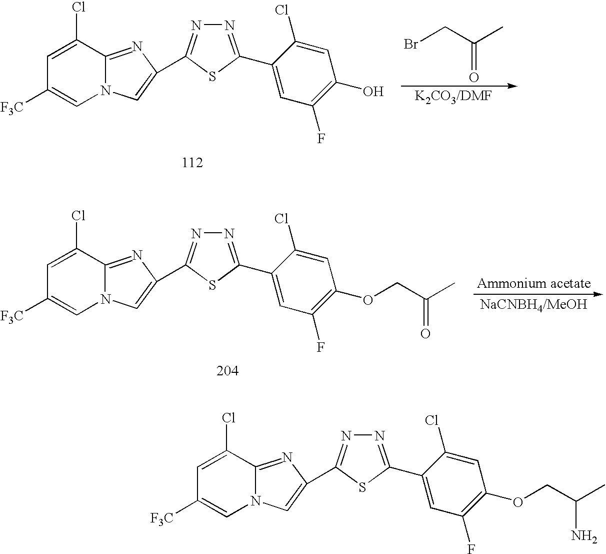

1-(5-Chloro-4-(5-(8-chloro-6-(trifluoromethyl)imidazo[1,2-a]pyridin-2-yl)-1,3,4-thiadiazol-2-yl)-2-fluorophenoxy)propan-2-one (204). To a stirred suspension of 112 (1.5 g, 3.3 mmol), prepared as described in Example 22, and K₂CO₃ (1.8 g, 13 mmol) in DMF (20 mL) was added bromoacetone (1.12 mL, 13 mmol) dropwise and the reaction mixture was then heated at 90° C. for 5 h. Solvent was then removed under reduced pressure and the reaction mixture was extracted with ethyl acetate. The organic layer was dried and concentrated and the resulting solid recrystallized from isopropyl alcohol to give 204 as an off-white solid (2 g, 118%).

1-[(5-Chloro-4-{5-[8-chloro-6-(trifluoromethyl)imidazo[1,2-a]pyridin-2-yl]-1,3,4-thiadiazol-2-yl}-2-fluorophenyl)oxy]propan-2-amine. To a stirred solution of 204 (2.0 g, 3.9 mmol) in MeOH (9 mL) was added NH₄OAc (0.9 g, 11.8 mmol) and the reaction mixture was stirred for 30 min at rt followed by addition of NaCNBH₄ (0.756 g, 12 mmol). The reaction mixture was then stirred for 48 h at rt. Solvent was removed from the reaction mixture and ice cold water was added. The resulting aqueous mixture was extracted with EtOAc. The organic layer was concentrated and the resulting residue purified by prep HPLC to give the title compound (20 mg, 1%). ¹H-NMR (400 MHz, DMSO-d₆) δ 9.4 (s, 1H), 8.9 (s, 1H), 8.2 (d, 1H), 8.0 (s, 3H), 7.7 (m, 1H), 4.4 (m, 2H), 4.2 (m, 1H), 1.3 (d, 3H); MS (EI) for C₁₉H₁₃Cl₂F₄N₅OS, found 505.8 (MH+).

### Example 71

### 2-Amino-3-[(5-chloro-2-fluoro-4-{5-[6-(trifluoromethyl)imidazo[1,2-a]pyridin-2-yl]-1,3,4-thiadiazol-2-yl}phenyl)oxy]-2-methylpropan-1-ol hydrochloride

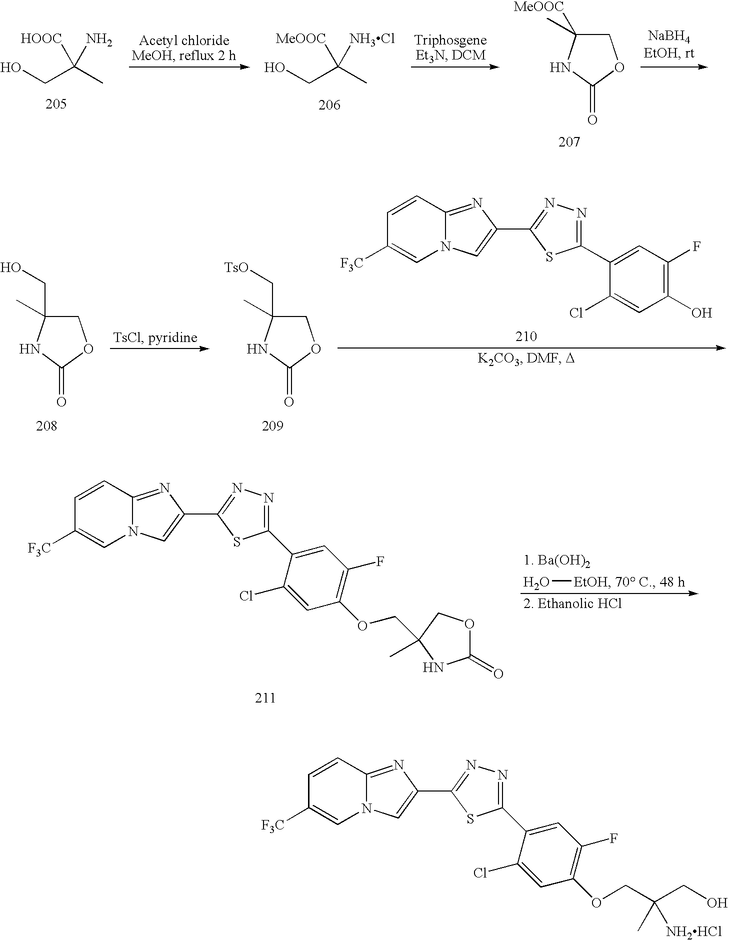

Methyl 2-amino-3-hydroxy-2-methylpropanoate hydrochloride (206). Acetyl chloride (10 mL, 138 mmol) was added dropwise over a period of 10 min to stirring MeOH (50 mL) at rt under N₂ atmosphere. The solution was stirred for a further 5 min, then 205 (5 g, 41 mmol) was added in one portion and the solution was slowly heated to reflux for 2 h. The solution was allowed to cool to room temperature and the solvent was removed under reduced pressure to give crude 12 as a white crystalline solid (8 g, 125%) which was used without further purification.

Methyl 4-methyl-2-oxooxazolidine-4-carboxylate (207). Triethylamine (8 g, 141 mmol) was added to a solution of 206 (8 g, 47 mmol) in DCM (60 mL) at 0° C. and stirred for 30 min. A solution of triphosgene (23.1 g) in DCM (10 mL) was added slowly over period of 40 min at 0° C. The reaction mixture was slowly allowed to warm to room temperature over 2 h with stirring. Hexane was added to the reaction mixture and stirred for 45 min. The reaction mixture was filtered and any insoluble material washed with EtOAc. The filtrate was concentrated to provide crude product which was purified by column chromatography on silica (50% EtOAc in hexanes) to afford 207 (3.3 g, 44%).

4-(Hydroxymethyl)-4-methyloxazolidin-2-one (208). NaBH₄ (0.94 g) was added in portions to a solution of ester 207 (3.3 g) in dry ethanol (20 mL) at 0° C. The reaction mixture was stirred at rt for 2.5 h. Aqueous saturated ammonium chloride (5 mL) was added and the resulting mixture stirred for 30 min at rt. The reaction mixture was filtered and the filtrate was concentrated to dryness to afford a white solid. Traces of water were removed by toluene azeotropes. The obtained crude product was purified by column chromatography (10% methanol in EtOAc) to give 208 (2.1 g, 77%).

(4-Methyl-2-oxooxazolidin-4-yl)methyl 4-methylbenzenesulfonate (209). Under N₂ atmosphere, p-toluenesulfonyl chloride was added to a solution of 208 (2.1 g) in pyridine (20 mL) at 0° C. The reaction was stirred at room temperature for 4 h. Pyridine was evaporated and resulting residue was dissolved in dichloromethane (25 mL). The organic layer was washed with 1N HCl solution (5 mL). The organic layer was concentrated and the resulting residue was washed with hot pentane to remove excess p-toluenesulfonyl chloride. The resulting residue was dissolved in a minimum amount dichloromethane and hexanes were added to precipitate the product. The resulting white solid was filtered and dried to afford 209 (3.8, 83%).

5-Chloro-2-fluoro-4-(5-(6-(trifluoromethyl)imidazo[1,2-a]pyridin-2-yl)-1,3,4-thiadiazol-2-yl)phenol (210). Compound 210 was synthesized in a manner analogous to compound 112 in Example 22.

4-((5-Chloro-2-fluoro-4-(5-(6-(trifluoromethyl)imidazo[1,2-a]pyridin-2-yl)-1,3,4-thiadiazol-2-yl)phenoxy)methyl)-4-methyloxazolidin-2-one (211). The mixture of 210 (0.7 g, 1.7 mmol), 209 (0.69 g, 2.4 mmol) and K₂CO₃ (0.45 g, 3.2 mmol) in DMF (5 mL) was heated to 80° C. for 4 h. The reaction mixture was cooled and quenched with ice water. The resulting solid was filtered, washed with cold acetone (4 mL) and dried to afford 211 (0.7 g, 78%).

2-Amino-3-[(5-chloro-2-fluoro-4-{5-[6-(trifluoromethyl)imidazo[1,2-a]pyridin-2-yl]-1,3,4-thiadiazol-2-yl}phenyl)oxy]-2-methylpropan-1-ol hydrochloride. A solution of Ba(OH)₂ (1.2 g, 3.9 mmol) in water (30 mL) was added to a solution of 211 (0.7 g, 1.3 mmol) in EtOH (15 mL). The reaction mixture was heated to 70° C. for 48 h. The reaction mixture was then diluted with water and filtered. To the resulting residue, ethanolic HCl (15 mL) was added and stirred for 1 h. The residue was filtered and dried. The obtained residue was diluted with NMP (5 mL), stirred for 30 min and again filtered. The residue was washed with MeOH (1 mL) and dried to afford the title compound (40 mg, 6%). ¹H-NMR (400 MHz, DMSO-d₆) δ 9.35 (s, 1H), 8.8 (s, 1H), 8.2 (m, 1H), 7.9 (d, 1H), 7.8 (d, 1H), 7.65 (m, 2H), 5.6 (t, 1H), 4.35 (m, 2H), 3.6 (m, 2H), 1.3 (s, 3H); MS (EI) for C₂₀H₁₆ClF₄N₅O₂S, found 501.9 (MH+).

Using the same or analogous synthetic techniques in Example 71 and substituting with appropriate reagents (prepared using procedures as described herein), 2-amino-3-[(5-chloro-4-{5-[8-chloro-6-(trifluoromethyl)imidazo[1,2-a]pyridin-2-yl]-1,3,4-thiadiazol-2-yl}-2-fluorophenyl)oxy]-2-methylpropan-1-ol was prepared. MS (EI) for C₂₀H₁₅Cl₂F₄N₅O₂S, found 535.7 (MH+).

### Example 72

### 8-Chloro-2-[3-(2,5-dichloro-4-{[(methylsulfonyl)methyl]oxy}phenyl)-1,2,4-oxadiazol-5-yl]-6-(trifluoromethyl)imidazo[1,2-a]pyridine

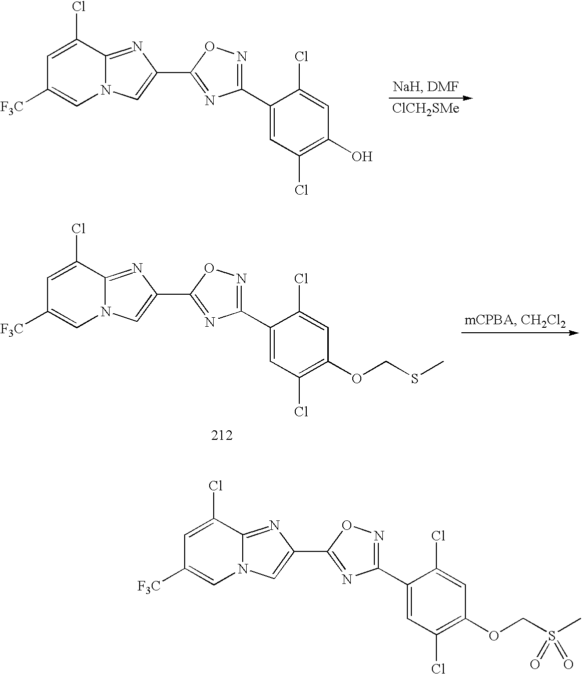

5-(8-Chloro-6-(trifluoromethyl)imidazo[1,2-a]pyridin-2-yl)-3-(2,5-dichloro-4-(methylthiomethoxy)phenyl)-1,2,4-oxadiazole (212). To a solution of 2,5-dichloro-4-(5-(8-chloro-6-(trifluoromethyl)imidazo(1,2-a)pyridine-2-yl)-1,2,4-oxadiazole-3-yl) phenol (300 mg, 0.6 mmol), prepared as described in Example 13, in DMF (3 mL) was added NaH (50 mg, 1.2 mmol, 60% in mineral oil). The mixture was stirred for 15 min at rt. Chloromethyl methyl sulfide (116 mg, 1.2 mmol) was added. The reaction was complete in 3 h. Water (20 mL) was added, and the product was extracted with EtOAc. The EtOAc solution was dried over Na₂SO₄. Removal of the solvent gave the crude sulfide 212.

8-Chloro-2-[3-(2,5-dichloro-4-{[(methylsulfonyl)methyl]oxy}phenyl)-1,2,4-oxadiazol-5-yl]-6-(trifluoromethyl)imidazo[1,2-a]pyridine. To a 0° C. solution of sulfide 212 in CH₂Cl₂ (3 mL) was added mCPBA (135 mg, 0.78 mmol, 77%). The mixture was stirred for 1 h at 0° C. and 2 h at rt. CH₂Cl₂ was removed. The solid residue was washed with sat. aqueous NaHCO₃, H₂O, MeOH and dried to give the title compound (87 mg, 27% over two steps). ¹H-NMR (400 MHz, DMSO-d₆) δ 9.33 (br s, 1H), 9.08 (s, 1H), 8.17 (s, 1H), 8.07 (d, 1H), 7.89 (s, 1H), 5.66 (s, 2H), 3.15 (s, 3H); MS (EI) for C₁₈H₁₀Cl₃F₃N₄O₄S, found 543.0 (MH+).

Using the same or analogous synthetic techniques in Example 72 and substituting with appropriate reagents (which are commercially available or prepared using procedures known to one of ordinary skill in the art), 8-chloro-2-[3-(2,5-dichloro-4-{[2-(methylsulfonyl)ethyl]oxy}phenyl)-1,2,4-oxadiazol-5-yl]-6-(trifluoromethyl)imidazo[1,2-a]pyridine was prepared. ¹H-NMR (400 MHz, DMSO-d₆) δ 9.35 (s, 1H), 9.05 (s, 1H), 8.15 (s, 1H), 8.05 (s, 1H), 7.65 (s, 1H), 4.60 (t, 2H), 3.75 (t, 2H), 3.15 (s, 3H); MS (EI) for C₁₉H₁₂Cl₃F₃N₄O₄S, found 555 (MH+).

### Example 73

### (1R)-2-[(2,5-Dichloro-4-{5-[8-chloro-6-(trifluoromethyl)imidazo[1,2-a]pyridin-2-yl]-1,2,4-oxadiazol-3-yl}phenyl)oxy]-1-methylethyl hydrogen sulfate

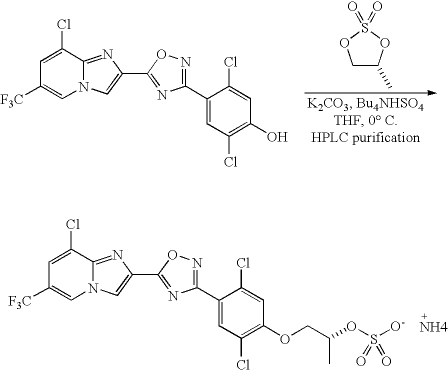

(1R)-2-[(2,5-Dichloro-4-{5-[8-chloro-6-(trifluoromethyl)imidazo[1,2-a]pyridin-2-yl]-1,2,4-oxadiazol-3-yl}phenyl)oxy]-1-methylethyl hydrogen sulfate ammonium salt. To a 0° C. solution of 2,5-dichloro-4-(5-(8-chloro-6-(trifluoromethyl)imidazo oxadiazol-3-yl)phenol (300 mg, 0.67 mmol), prepared as described in Example 13, in THF (5 mL) were added Bu₄NHSO₄ (45 mg, 0.13 mmol), K₂CO₃ (277 mg, 2.0 mmol) and (R)-1-Methyl-1,2-ethylene sulfate (184 mg, 1.33 mmol), prepared according to a literature procedure described in Tetrahedron: Asymmetry 1998, 2233. The reaction was complete in 2 h. K₂CO₃ was filtered off. Concentration of the filtrate and purification by HPLC gave the desired product (87 mg, 22%). ¹H-NMR (400 MHz, DMSO-d₆) δ 9.33 (s, 1H), 9.09 (s, 1H), 8.11 (s, 1H), 8.07 (s, 1H), 7.58 (s, 1H), 7.09 (br s, 4H), 4.52 (m, 1H), 4.28 (d, 2H), 1.30 (d, 3H); MS (EI) for C₁₉H₁₁Cl₃F₃N₄O₆S, found 587.0 (MH+).

### Example 74

### N-[(3-Chloro-4-{5-[8-chloro-6-(trifluoromethyl)imidazo[1,2-a]pyridin-2-yl]-1,3,4-thiadiazol-2-yl}phenyl)methyl]methanesulfonamide

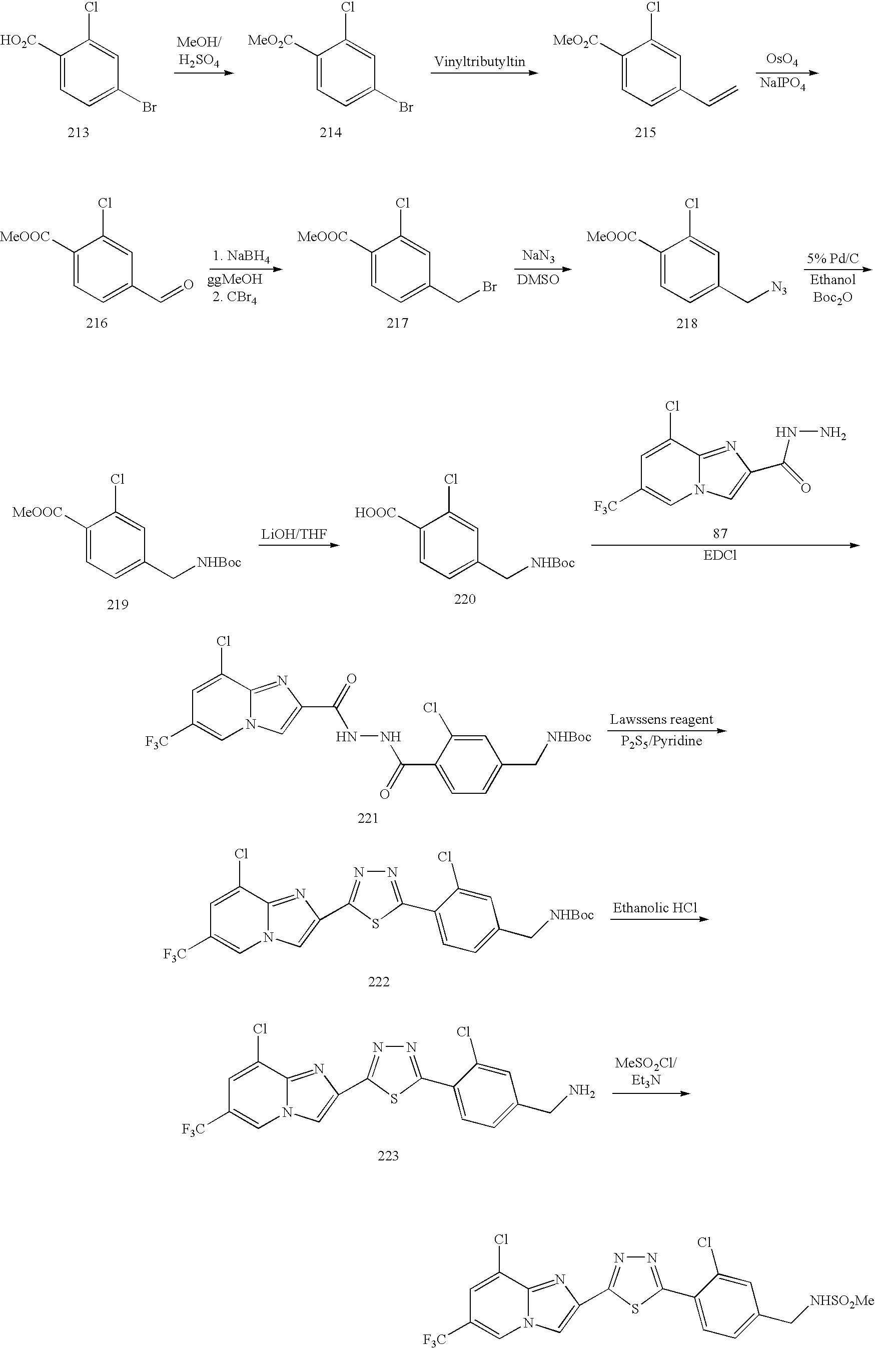

Methyl 4-bromo-2-chlorobenzoate (214). To a stirred solution of 213 (10 g, 42.55 mmol) in methanol (200 mL) was added conc. H₂SO₄ (10 mL) at 0° C. dropwise. After addition, it was heated to 80° C. for 3 h. The reaction mixture was concentrated under vacuum. The resulting residue was dissolved in ethyl acetate and washed with water, sodium bicarbonate solution and brine. The organic layer was dried over anhydrous sodium sulfate and concentrated under vacuum to afford 214 (9.5 g, 90%).

Methyl 2-chloro-4-vinylbenzoate (215). To a stirred solution of 214 (9.5 g, 38.15 mmol) in DMF (250 mL) was added LiCl (4.8 g, 114.45 mmol) and tributylvinyl tin (1.21 g, 38.15 mmol). The reaction mixture was degassed with argon for 20 min. To the reaction mixture, PdCl₂(PPh₃)₂ (2.14 g, 3.05 mmol) was added and the mixture again degassed with argon for 20 min. The reaction mixture was heated to 110° C. for 15 h. After completion, solvent was removed at reduced pressure and the resulting residue was partitioned between water and ethyl acetate. The phases were separated and the organic phase was dried over sodium sulfate and concentrated under vacuum. The crude compound was purified by column chromatography to afford 215 (6.2 g, 82%).

Methyl 2-chloro-4-formylbenzoate (216). A mixture of 215 (5 g, 16.66 mmol), acetone (45 mL), water (5 mL), NMO (4 mL) and OsO₄ (1.3 mL, 0.1 M solution in toluene) was stirred at room temperature for 16 h. The reaction was diluted with EtOAc and washed with brine. The organic layer was dried over sodium sulfate and concentrated under reduced pressure. The crude product was stirred in n-pentane (10 mL) for 15 min and the resulting solids were filtered and then resuspended in THF:Water (2:1) (6 mL) and NaIO₄ (7.13 g, 33.32 mmol) was added. The reaction mixture was stirred at rt for 2 h. The resulting solids were filtered, washed with water and dried to obtain aldehyde 216 (4.5 g, 90%) which was used as such for the next step.

Methyl 4-(bromomethyl)-2-chlorobenzoate (217). To a stirred solution of 216 (4.5 g, 22.61 mmol) in methanol (50 mL) was added NaBH₄ (0.855 g, 22.61 mole) at 0° C. The reaction mixture was stirred at 0° C. for 30 min. The reaction mixture was concentrated under reduced pressure and the resulting residue was diluted with cold water. The reaction mixture was extracted with ethyl acetate and the organic layer was washed with water and brine, dried over sodium sulfate and concentrated to afford alcohol (4.12 g, 91%). To a stirred solution of alcohol in DCM (35 mL) was added triphenylphosphine (8.06 g, 30.75 mmol) at 0° C. and stirred for 5 min. Carbon tetrabromide (8.14 g, 24.6 mmol) was added at 0° C. in portions over 15 min and the reaction mixture further stirred for 10 min, then allowed to stir at room temperature for 12 h. The reaction mixture was concentrated under vacuum and purified by column chromatography to afford 217 (3.4 g, 63%).

Methyl 4-(azidomethyl)-2-chlorobenzoate (218). To a stirred solution of 217 (3.4 g, 12.87 mmol) in dry DMSO (20 mL) was added NaN₃ (1.09 g, 16.73 mmol) at room temperature. The reaction mixture was stirred at room temperature for 12 h. After completion, the reaction mixture was diluted with water and extracted with ethyl acetate. The organic layer was washed with water and brine solution, dried over Na₂SO₄ and concentrated under vacuum to give 218 (2.8 g, 96%).

Methyl 4-((tert-butoxycarbonylamino)methyl)-2-chlorobenzoate (219). To a stirred solution of 218 (2.8 g, 12.39 mmol) in ethanol (10 mL) was added (Boc)₂O (2.7 g, 12.39 mmol) followed by 5% Pd/C (0.28 g). The reaction mixture was stirred at room temperature overnight under an atmosphere of hydrogen. The reaction mixture was filtered and the filtrate was concentrated in vacuo. The crude compound was diluted with ethyl acetate and washed with water, brine, dried over Na₂SO₄ and concentrated under vacuum to give 219 (2.6 g, 70%).

4-((tert-Butoxycarbonylamino)methyl)-2-chlorobenzoic acid (220). A mixture of 219 (2.6 g, 8.66 mmol), ethanol (10 mL) and 2M lithium hydroxide solution (0.91 g, 21.66 mmol) was stirred at room temperature for 3 h. After completion, the reaction mixture was concentrated under vacuum to remove ethanol and the pH was adjusted to acidic by dropwise addition of citric acid solution at 0° C. The reaction mixture was extracted with ethyl acetate, dried over Na₂SO₄ and concentrated under vacuum to give 220 (2 g, 80%).

tert-Butyl 3-chloro-4-(2-(8-chloro-6-(trifluoromethyl)imidazo[1,2-a]pyridine-2-carbonyl)hydrazinecarbonyl)benzylcarbamate (221). A mixture of acid 220 (2 g, 7 mmol), DMF (20 mL) and EDCI (1.87 g, 9.8 mmol) was stirred at rt for 15 min, then hydrazide 87 (2.34 g, 8.4 mmol) was added and the resulting mixture further stirred at room temperature for 14 h. After completion, water was added and the resulting solid was filtered, washed with isopropanol and dried to give 221 (1.8 g, 47%) which was used as such for next step.

tert-Butyl 3-chloro-4-(5-(8-chloro-6-(trifluoromethyl)imidazo[1,2-a]pyridin-2-yl)-1,3,4-thiadiazol-2-yl)benzylcarbamate (222). A mixture of 221 (1.8 g, 3.3 mmol), toluene (30 mL), pyridine (0.521 g, 6.6 mmol) and Lawesson's reagent (1.73 g, 4.3 mmol) was stirred at 120° C. for 4 h. The reaction mixture was cooled to room temperature and solvent was removed. The solid obtained was mixed with pyridine (30 mL) and phosphorous pentasulfide (2.8 g, 12.87 mmol) and again stirred at 120° C. for 3 h. Pyridine was removed and the resulting residue was partitioned between water and EtOAc. The organic layer was dried and concentrated to afford 222 (1.2 g, 66%) which was used as such for the next step.

(3-Chloro-4-(5-(8-chloro-6-(trifluoromethyl)imidazo[1,2-a]pyridin-2-yl)-1,3,4-thiadiazol-2-yl)phenyl)methanamine (223). A stirred solution of 222 (1.0 g, 1.83 mmol) in ethanolic HCl (5 mL) was stirred for 1 h at room temperature. After completion, the reaction mixture was concentrated under vacuum and the resulting residue was washed with hexane to afford amine 223 (0.3 g, 37%) which was used as such for the next step.

N-[(3-Chloro-4-{5-[8-chloro-6-(trifluoromethyl)imidazo[1,2-a]pyridin-2-yl]-1,3,4-thiadiazol-2-yl}phenyl)methyl]methanesulfonamide. To a stirred mixture of amine 223 (0.3 g, 0.67 mmol), dichloromethane (10 mL) and triethylamine (0.3 mL, 2.01) was added dropwise methanesulfonyl chloride (0.116 mL, 1.05 mmol) at 0° C. The resulting mixture was stirred for 10 min at 0° C. then allowed to stir at rt for 1.5 h. Upon completion, the reaction mixture was poured into water and extracted with DCM. The combined DCM layers were washed with water and brine solution, dried over Na₂SO₄ and concentrated under vacuum. The crude compound was purified by prep HPLC to give the title compound (21 mg, 6%). ¹H-NMR (400 MHz, DMSO-d₆) δ 9.35 (s, 1H), 8.95 (s, 1H), 8.25 (d, 1H), 8 (s, 1H), 7.9 (m, 2H), 7.55 (d, 1H), 4.3 (m, 2H), 3.0 (s, 3H); MS (EI) for C₁₈H₁₂C₁₂F₃N₅O₂S₂, found 521.8 (MH+).

### Example 75

### 2-Amino-2-[2-(5-chloro-4-{5-[8-chloro-6-(trifluoromethyl)imidazo[1,2-a]pyridin-2-yl]-1,2,4-oxadiazol-3-yl}-2-fluorophenyl)ethyl]propane-1,3-diol

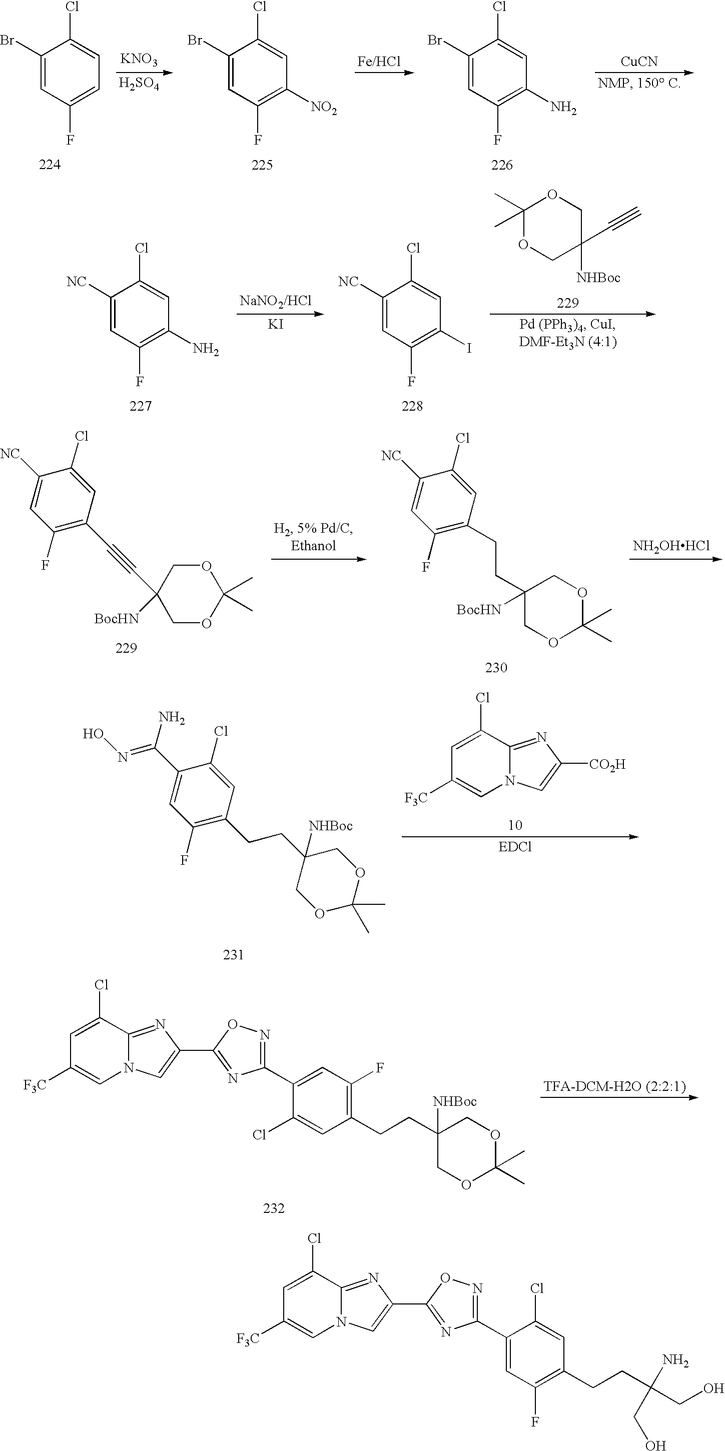

1-Bromo-2-chloro-5-fluoro-4-nitrobenzene (225). To a solution of 224 (25 g, 119.36 mmol) in H₂SO₄ (200 mL) was added KNO₃ (12.05 g, 119.36 mmol) at 0° C. After 15 min, the reaction mixture was allowed to warm to room temperature and stirred for 4 h. The reaction mixture was then poured into ice water and extracted with EtOAc. The combined organic layers were dried over anhydrous sodium sulfate, and concentrated under reduced pressure to afford 225 (28 g, 92%) which was used as such for the next step.

4-Bromo-5-chloro-2-fluoroaniline (226). To a mixture of 225 (28 g, 110 mmol), EtOH (125 mL) and conc HCl (112 mL) was added iron powder (58 g, 1.03 mmol) in portions over a period of 1 h at 0° C. The reaction mixture was then stirred at room temperature for 1 h. After completion, reaction mixture was diluted with EtOAc and made basic with saturated NaHCO₃ solution. The resulting suspension was filtered through a bed of celite and the resulting layers of the filtrate were separated. The organic layer was washed with water, dried over anhydrous sodium sulfate and concentrated under reduced pressure to give 226 (23 g, 93%).

4-Amino-2-chloro-5-fluorobenzonitrile (227). A mixture of 226 (6 g, 26.73 mmol), CuCN (4.81 g, 53.46 mmol) and DMF (40 mL) was stirred at 150° C. for 6 h. After cooling, the reaction mixture was partitioned between water and EtOAc. The resulting suspension was filtered through celite and the layers of the filtrate were separated. The organic layer was washed with saturated sodium bicarbonate, dried over anhydrous sodium sulfate and concentrated under reduced pressure. The crude product was purified by column chromatography to give 227 (3.1 g, 68%).

2-Chloro-5-fluoro-4-iodobenzonitrile (228). A solution of 227 (3 g, 13.36 mmol) in conc HCl (10 mL) was cooled to 0° C. To this, a cold aqueous solution of NaNO₂ (6.0 g, 86.95 mmol) in water (25 mL) was added and the reaction mixture was stirred at 0° C. for another 30 min. The resulting cold solution of diazonium salt was slowly added to a solution of potassium iodide in water (95 mL) at 0° C. and stirred for 15 min, followed by stirring at room temperature for 12 h. The reaction mixture was then diluted with EtOAc and filtered through celite. The filtrate was concentrated and purified by column chromatography to give 228 (3.4 g, 68%).

tert-Butyl 5-((5-chloro-4-cyano-2-fluorophenyl)ethynyl)-2,2-dimethyl-1,3-dioxan-5-ylcarbamate (229). A mixture of 228 (3 g, 10.67 mmol), 229 (2.99 g, 11.73 mmol) and triethylamine (8 mL) in DMF (32 mL) was degassed for 30 min, then Pd(PPh₃)₄ (0.616 g, 0.53 mmol) and CuI (0.202 g, 1.067 mmol) were added to the reaction mixture with further degassing. The reaction mixture was stirred at room temperature for 4 h. The reaction was quenched by adding 10% KF (50 mL) and stirred for 30 min. The product was extracted in EtOAc. The organic layer was washed with water, saturated sodium bicarbonate, dried over sodium sulfate, concentrated under reduced pressure and purified by column chromatography to give 229 (2 g, 46%).

tert-Butyl 5-(5-chloro-4-cyano-2-fluorophenethyl)-2,2-dimethyl-1,3-dioxan-5-ylcarbamate (230). To a solution of 229 (2 g, 4.9 mmol) in EtOH (50 mL) was added 10% Pd—C (800 mg). Under H₂ atmosphere, the reaction mixture was stirred at room temperature for 3 days while maintaining 60 psi pressure. The reaction mixture was filtered through a bed of celite and the filtrate was concentrated to afford 230 (1 g, 49%) which was used as such in subsequent reactions without further purification.

tert-Butyl 5-(5-chloro-2-fluoro-4-(N′-hydroxycarbamimidoyl)phenethyl)-2,2-dimethyl-1,3-dioxan-5-ylcarbamate (231). A mixture of hydroxylamine hydrochloride (0.84 g, 12.11 mmol), ethanol (10 mL) and triethylamine (2.3 mL, 16.95 mmol) was stirred at room temperature for 30 min. To the mixture, 230 (1.0 g, 2.4 mmol) was added in one portion and the reaction was stirred at 85° C. for 2 h. Solvent was removed under vacuum. The resulting residue was diluted in water, extracted with ethyl acetate (4×25 mL), dried over sodium sulfate and concentrated under vacuum. The crude product was dried azeotropically with toluene to obtain 231 (1 g, 110%) which was used as such without further purification.

tert-Butyl 5-(5-chloro-4-(5-(8-chloro-6-(trifluoromethyl)imidazo[1,2-a]pyridin-2-yl)-1,2,4-oxadiazol-3-yl)-2-fluorophenethyl)-2,2-dimethyl-1,3-dioxan-5-ylcarbamate (232). A mixture of acid 10 (0.595 g, 2.24 mmol), DMF (20 mL), EDCI (0.517 g, 2.7 mmol) and HOBT (0.363 g, 2.7 mmol) was stirred at rt for 45 min. Amidoxime 231 (1.0 g, 2.24 mmol) was added to the reaction mixture and it was heated to 100° C. for 12 h. The solvent was then removed under vacuum, water was added and the resulting suspension was stirred for 30 min. The resulting solid was filtered, azeotropically dried with toluene, resuspended in isopropyl alcohol (15 mL), stirred for 30 min, filtered, and dried to afford 232 (0.5 g, 36% two steps).

2-Amino-2-[2-(5-chloro-4-{5-[8-chloro-6-(trifluoromethyl)imidazo[1,2-a]pyridin-2-yl]-1,2,4-oxadiazol-3yl}-2-fluorophenyl)ethyl]propane-1,3-diol. To a solution of 232 (0.5 g, 0.74 mmol) in dichloromethane (4 mL) was added trifluoroacetic acid (0.8 g). The reaction mixture was stirred for 1 h, then filtered through a bed of celite and the filtrate was concentrated. The obtained crude product was purified by prep HPLC to give the title compound (0.1 g, 25%). ¹H-NMR (400 MHz, DMSO-d₆) δ 9.35 (s, 1H), 9.05 (s, 1H), 8.05 (s, 1H), 7.9 (d, 1H), 7.8 (bs, 2H), 7.7 (d, 1H), 5.4 (m, 2H), 3.55 (m, 4H), 2.8 (m, 2H), 1.9 (m, 2H); MS (EI) for C₂₁H₁₇C₁₂F₄N₅O₃, found 533.9 (MH+).

### Example 76

### 2-[(2,5-Dichloro-4-{5-[8-chloro-6-(trifluoromethyl)imidazo[1,2-a]pyridin-2-yl]-1,2,4-oxadiazol-3-yl}phenyl)oxy]-1-(hydroxymethyl)ethyl dihydrogen phosphate

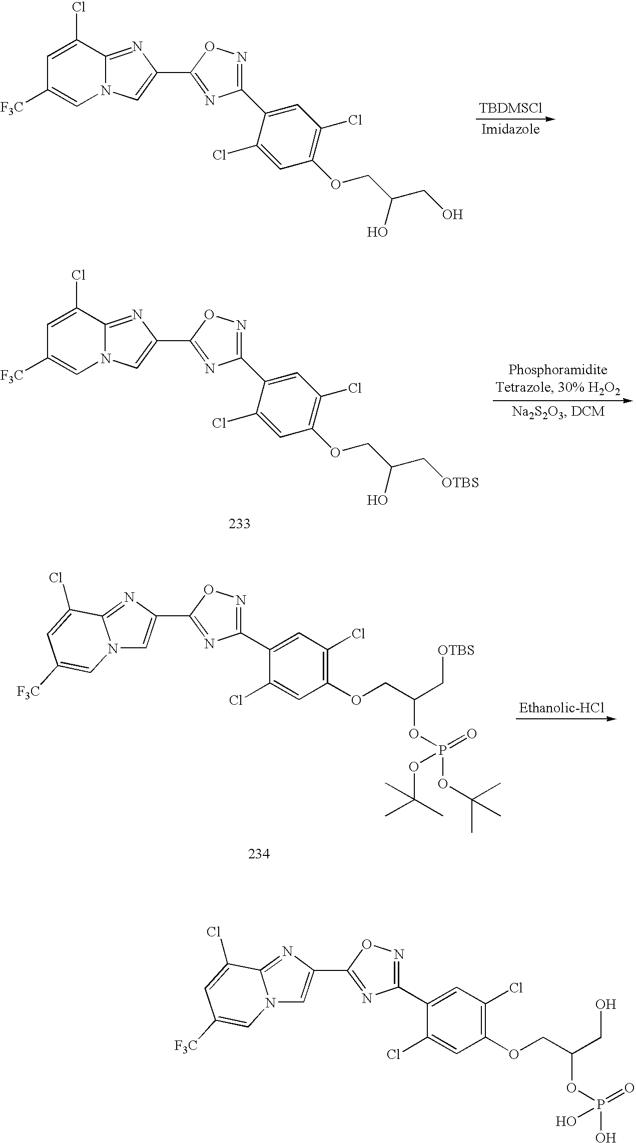

1-(tert-Butyldimethylsilyloxy)-3-(2,5-dichloro-4-(5-(8-chloro-6-(trifluoromethyl)imidazo[1,2-a]pyridin-2-yl)-1,2,4-oxadiazol-3-yl)phenoxy)propan-2-ol (233). To a solution of 3-[(2,5-dichloro-4-{5-[8-chloro-6-(trifluoromethyl)imidazo[1,2-a]pyridin-2-yl]-1,2,4-oxadiazol-3-yl}phenyl)oxy]propane-1,2-diol (3.8 g,7.3 mmol), prepared as described in Example 14, imidazole (1.48 g, 21.8 mmol) and DMAP (0.2 g, 0.029 mmol) in CH₂Cl₂ (40 mL) was added TBDMSCl (1.32 g, 8.75 mmol) in portions at 0° C. The reaction mixture was then allowed to stir at room temperature for 4 h, diluted with dichloromethane and washed with saturated NaHCO₃ solution. The organic layer was dried over sodium sulfate, concentrated under reduced pressure and the crude product purified by column chromatography to afford 233 (2.7 g, 58%).

di-tert-Butyl 1-(tert-butyldimethylsilyloxy)-3-(2,5-dichloro-4-(5-(8-chloro-6-(trifluoromethyl)imidazo[1,2-a]pyridin-2-yl)-1,2,4-oxadiazol-3-yl)phenoxy)propan-2-yl phosphate (234). Under N₂, phosphoramidite (0.66 mL, 2.36 mmol) was added to a solution of 233 (0.6 g, 0.95 mmol) in dichloromethane (20 mL) at 0° C. To this, 1-H tetrazole (0.165 g, 2.36 mmol) was added and the mixture stirred at 0° C. for 5 min. The reaction was then allowed to stir at room temperature for 2 h. The reaction mixture was recooled to 0° C. and 30% H₂O₂ was added. After 30 min, saturated Na₂S₂O₃ solution was added and stirring was continued at 0° C. for 1.5 h. The reaction mixture was then diluted with water and extracted with EtOAc. The combined organic layers were dried over sodium sulfate and concentrated under reduced pressure to afford 234 (0.8 g) which was used as such for the next step.

2-[(2,5-Dichloro-4-{5-[8-chloro-6-(trifluoromethyl)imidazo[1,2-a]pyridin-2-yl]-1,2,4-oxadiazol-3-yl}phenyl)oxy]-1-(hydroxymethyl)ethyl dihydrogen phosphate. A solution of compound 234 (0.8 g) in ethanolic HCl (5 mL) was stirred at room temperature. After 1 h, solvent was removed under reduced pressure and the resulting residue was purified by prep HPLC to afford the title compound (230 mg, 34% two steps). ¹H-NMR (400 MHz, CD₃OD) δ 9.2 (s, 1H), 8.95 (s, 1H), 8.1 (s, 1H), 7.8 (s, 1H), 7.4 (s, 1H), 4.6 (m, 1H), 4.4 (m, 2H), 3.9 (m, 3H); MS (EI) for C₁₉H₁₃C₁₃F₃N₄O₇P, found 603 (MH+).

The following compounds were prepared using the same or analogous synthetic techniques in Example 76 and substituting with appropriate reagents, which were commercially available or were prepared using procedures herein or procedures known to one of ordinary skill in the art. 3-(2,5-Dichloro-4-{5-[8-chloro-6-(trifluoromethyl)imidazo[1,2-a]pyridin-2-yl]-1,2,4-oxadiazol-3-yl}phenyl)-1-methylpropyl dihydrogen phosphate. MS (EI) for C₂₀H₁₅Cl₃F₃N₄O₅P, found 585.1 (MH+). 3-[(2,5-Dichloro-4-{5-[8-chloro-6-(trifluoromethyl)imidazo[1,2-a]pyridin-2-yl]-1,2,4-oxadiazol-3-yl}phenyl)oxy]-2-oxopropyl dihydrogen phosphate. MS (EI) for C₁₉H₁₁Cl₃F₃N₄O₇P, found 600.8 (MH+). 3-[(2,5-Dichloro-4-{548-chloro-6-(trifluoromethyl)imidazo[1,2-a]pyridin-2-yl]-1,2,4-oxadiazol-3-yl}phenyl)oxy]-2-hydroxypropyl dihydrogen phosphate. MS (EI) for C₁₉H₁₃Cl₃F₃N₄O₇P, found 603.2 (MH+). 2-Amino-3-[(2,5-dichloro-4-{5-[8-chloro-6-(trifluoromethyl)imidazo[1,2-a]pyridin-2-yl]-1,2,4-oxadiazol-3-yl}phenyl)oxy]propyl dihydrogen phosphate. ¹H-NMR (400 MHz, DMSO-d₆) δ 9.00 (s, 1H), 8.20 (s, 1H), 8.15 (s, 1H), 7.90 (s, 1H), 7.60 (s, 1H), 4.50 (m, 1H), 42.5 (m, 4H).

### Example 77

### {[(2,5-Dichloro-4-{5-[8-chloro-6-(trifluoromethyl)imidazo[1,2-a]pyridin-2-yl]-1,2,4-oxadiazol-3-yl}phenyl)oxy]methyl}phosphonic acid

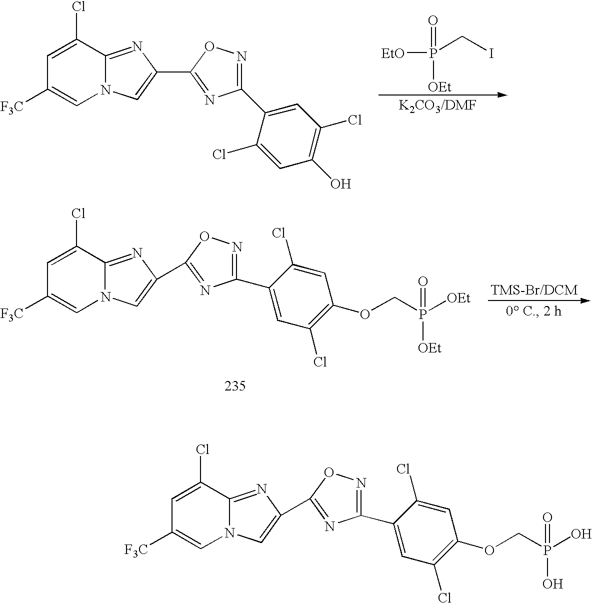

Diethyl(2,5-dichloro-4-(5-(8-chloro-6-(trifluoromethyl)imidazo[1,2-a]pyridin-2-yl)-1,2,4-oxadiazol-3-yl)phenoxy)methylphosphonate (235). To a stirred solution of 2,5-dichloro-4-(5-(8-chloro-6-(trifluoromethyl)imidazo(1,2-a)pyridine-2-yl)-1,2,4-oxadiazole-3-yl)phenol (1.5 g, 0.0033 mol), prepared as described in Example 13, in DMF (20 mL) was added diethyl(α-iodomethyl)phosphonate (3.7 g, 0.0133 mol) and K₂CO₃ (1.8 g, 0.0133 mol). The resulting reaction mixture was heated to 90° C. for 14 h. After completion, the reaction mixture was cooled to room temperature and solvent was removed under reduced pressure. The crude compound was purified by column chromatography to afford 235 (0.5 g, 25%).

{[(2,5-Dichloro-4-{5-[8-chloro-6-(trifluoromethyl)imidazo[1,2-a]pyridin-2-yl]-1,2,4-oxadiazol-3-yl}phenyl)oxy]methyl}phosphonic acid. To a stirred solution of 235 (0.5 g, 0.00083 mol) in dry DCM (10 mL) was added TMS-Br (2.0 mL, 0.0151 mol) dropwise at 0° C. The reaction mixture was stirred at 0° C. for 2 h, then concentrated under vacuum. The resulting residue was dissolved in ethyl acetate and washed with water, brine, dried over anhydrous sodium sulfate and concentrated under vacuum. The crude compound was purified by prep HPLC to give the title compound (20 mg, 4%). ¹H-NMR (400 MHz, DMSO-d₆) δ 9.3 (s, 1H), 9.1 (s, 1H), 8.05 (s, 1H), 8.03 (s, 1H), 7.7 (s, 1H), 4.4 (d, 2H); MS (EI) for C₁₇H₉C₁₃F₃N₄O₅P, found 542.8 (MH+).

### Biological Examples

All Compounds in Table 1 were tested in one or more of the following biological assays and were found to be active as agonists of S1P1.

### Biological Example 1

### CNG cAMP Assay

Frozen HEK293 cells expressing the CNG channel and S1P₁ (BD Biosciences, San Jose, Calif.) were thawed and plated into the wells of a black, clear bottom, 384-well CellBind plate (Corning, Corning, N.Y.) at 14,000 cells per well. HEK293 cells expressing the CNG channel and CB1 (BD Biosciences) were cultured and plated under the same conditions. The cells were incubated for 16 h at 37° C. in complete DMEM medium (Invitrogen Carlsbad, Calif.) containing 10% FBS (HyClone Logan, Utah), 250 μg/mL geneticin (Invitrogen), and 1 μg/mL puromycin (Sigma-Aldrich, St. Louis, Mo.). A membrane potential dye (BD Biosciences) was added and the plates were incubated for 2-2.5 h at room temperature.

Test compounds were tested at maximum concentrations of 10 μM. Compounds were diluted in DMSO (10 concentration points, 3-fold each) and added to the assay plate at final DMSO concentrations of 1.8%. For each compound, there were duplicate assay plates and each assay plate had duplicate wells per concentration point. Test compounds were added to the cells in a DPBS solution containing 25 μM Ro 20-1724 (Sigma-Aldrich), 500 nM of the A2b receptor agonist NECA (Sigma-Aldrich) and 10 nM (EC₉₅) of S1P (Avanti Alabaster, Ala.) and incubated for 90 min. The assay plate was read before compound addition (T₀) and after the 90 min incubation (T₉₀) using an EnVision plate reader (PerkinElmer, Waltham, Mass.) at an excitation wavelength of 350 nm and an emission wavelength of 590 nm. The T₉₀/T₀ ratio was determined for each concentration of the test compounds. The percent agonist activity was determined as [(test compound−DMSO alone control)/(NECA alone control−DMSO alone control)*100]. The percent activities were plotted against compound concentration to determine EC₅₀ using XLFit (IDBS, Alameda, Calif.). The control used for calculating rEC50 in the S1P₁ CNG agonist assay was DMSO.

### Biological Example 2

### β-Arrestin Recruitment Assay

For the Tango™ β-arrestin recruitment assay, the cytoplasmic C-terminus of S1P₁ is tethered to the tTA transcriptional activator with a linker that contains a cleavage site for the N1a protease from tobacco etch virus (TEV protease). The C-terminus of the human β-arrestin2 protein is fused to TEV protease. Binding of an agonist recruits the β-arrestin-TEV fusion protein to the receptor resulting in cleavage of the linker and release of tTA to enter the nucleus and subsequently activate a tTA-dependent luciferase reporter gene.

Frozen HEK293 cells transiently transfected with receptor cDNAs for S1P₁ (Invitrogen) were thawed and suspended in 10 mL of Pro293a-CDM culture medium (Invitrogen) supplemented with 4 mM L-Glutamine (Invitrogen), 1× Pen/Strep (100 units/mL penicillin and 100 μg/mL streptomycin, Invitrogen) and 0.1% fatty acid free BSA (Sigma-Aldrich). Cells were added to the wells of a 384-well white opaque bottom assay plate (PerkinElmer) at 3,000-6,000 cells per well and the plate was incubated for approximately 4 h in a 37° C. incubator. Test compounds were tested at maximum concentrations of 10 μM for the agonist assays. Compounds were diluted in DMSO (10 concentration points, 3-fold each) and added to the assay plate at a 1% final DMSO concentration. For each compound, there were duplicate assay plates and each assay plate had duplicate wells per concentration point. The plate was incubated at 37° C. for 30 min. The efficacy control was 5 μM S1P (Avanti). Following agonist addition, the assay plates were incubated in a 37° C. incubator for 16-18 h. Luciferase assay reagent was added and luminescence measured in an EnVision plate reader (PerkinElmer). To determine agonist activity, percent activity was calculated as [(test compound−background)/(positive control−background)* 100], where background is the luminescence of the DMSO alone control and the positive control is the luminescence from cells incubated with the efficacy control 5 μM S1P. The percent activities were plotted against compound concentration to determine EC₅₀ using XLFit (IDBS).

Alternatively, U2OS cells expressing the reporter gene and S1P₁ (Invitrogen) were added to the wells of a 384-well white opaque bottom assay plate (PerkinElmer) at 0.3125×10⁶ cells per well. The cells were serum starved for 48 h in Freestyle medium (Invitrogen). Test compounds were tested at maximum concentrations of 1 μM for the agonist assay. Compounds were diluted in DMSO (10 concentration points, 3-fold each) and added to the assay plate at a 1% final DMSO concentration. The efficacy control was 1 μM S1P (Avanti). For each compound, there were duplicate assay plates and each assay plate had duplicate wells per concentration point. The plate was incubated overnight at 37° C. The GeneBLAzer β-lactamase assay reagent (Invitrogen) was added and the plates were incubated for an additional 2 h at room temperature. Fluorescence was measured using an EnVision plate reader (PerkinElmer, Waltham, Mass.) at an excitation wavelength of 409 nm and emission wavelengths of 460 nm and 530 nm. The emission intensity at each wavelength was background subtracted against wells containing medium only and the F_{460 nm}/F_{530 nm} ratio determined for each concentration of the test compounds. Percent activity was calculated as [(test compound ratio−DMSO ratio)/(positive control ratio−DMSO ratio)*100], where the positive control and DMSO ratios are from cells incubated with the efficacy control 1 μM S1P and 1% DMSO, respectively. The percent activities were plotted against compound concentration to determine EC₅₀ using XLFit (IDBS).

### Biological Example 3

### hS1P1R GTPγS and GTP-Eu Binding Assays

The hS1P1R GTPγS binding assay was carried out at room temperature in 96 well non-binding surface assay plates. The reaction in each well contained 4 μg hS1P1R (hEdg1) membrane protein (Lonza), 30 μM GDP, 0.1 nM [³⁵S]GTPγS, 0.25% fatty acid free BSA, and serially diluted hS1P1R agonist compound in 200 μL assay buffer (25 mM Tris-HCl PH 7.9, 100 mM NaCl, 3 mM MgCl2, and 0.2 mM EGTA). After one hour of incubation, 0.9 mg of WGA (Wheat Germ Agglutinin) SPA beads in 50 μL of assay buffer was added to each well. The SPA beads were spun down after an additional one hour incubation. The radioactivity of the bound GTPγS was counted by reading the assay plate using a MicroBeta.

The DELFIA GTP-Eu binding assay (PerkinElmer) is a time-resolved fluorometric assay based on GDP-GTP exchange. CHO cell membranes (Lonza) expressing human S1P₁ were incubated in 96-well filter plates (Pall, East Hills, N.Y.) in a final volume of 100 μL/well buffer containing 40 μg/mL membrane, 50 mM HEPES, 2 μM GDP, 10 mM MgCl₂, 100 mM NaCl, 500 μg/mL Saponin and test compound. Test compounds were tested at maximum concentrations of 10 μM. Compounds were diluted (10 concentration points, 3-fold each) and added to the assay plate at a 1% final DMSO concentration. For each compound, there were duplicate assay plates and each assay plate had duplicate wells per concentration point. The plates were incubated for 30 min at room temperature on a plate shaker at low speed. GTP-Eu was added to each well (10 μL, 10 nM final concentration) and the plate was incubated for an additional 30 min with slow shaking The wells were washed with ice cold GTP washing buffer (3×150 μL) using a vacuum manifold and the assay plates read in an EnVision plate reader (PerkinElmer) at an excitation wavelength of 340 nm and an emission wavelength of 615 nm. To determine agonist activity, percent activity was calculated as [(test compound−background)/(positive control−background)*100], where background is the fluorescence in absence of compound and the positive control is the fluorescence from membranes incubated with 1 μM S1P (Avanti). The percent activities were plotted against compound concentration to determine IC₅₀ or EC₅₀ using XLFit (IDBS).

### S1P1 Agonist Activity

Assay 1 is the CNG cAMP Assay as described in Biological Example 1. Assay 2a and 2b are the Tango™ β-arrestin Recruitment Assay in HEK293 and U2OS cells, respectively, as described in Biological Example 2. Assay 3a is the hS1P1R GTPγS Binding Assay as described in Biological Example 3. Assay 3b is the GTP-Eu Binding Assay as described in Biological Example 3. EC₅₀'s were measured unless otherwise noted. “A” means the compound has an EC₅₀ or relative EC₅₀ of less than or equal to 100 nM. “B” means the compound has an EC₅₀ or relative EC₅₀ greater than 100 nM but less than or equal to 500 nM. “C” means the compound has an EC₅₀ or relative EC₅₀ greater than 500 nM but less than or equal to 1 μM. “D” means the compound has an EC₅₀ or relative EC₅₀ greater than 1 μM but less than or equal to 5 μM. “E” means the compound has an EC₅₀ or relative EC₅₀ greater than 5 μM but less than or equal to 10 μM. In the table, “nt” means the Compound was not tested and “no” means the compound was tested but had no measurable activity under the assay conditions employed.

TABLE 4

| Entry | | | Assay | Assay | Assay | Assay |
|---|---|---|---|---|---|---|
| No. | ACD-generated Name | Assay 1 | 2a | 2b | 3a | 3b |
| 1 | 3-(3-chloro-4-{5-[8-chloro-6- | A | A | nt | nt | C |
| | (trifluoromethyl)imidazo[1,2-a]pyridin-2-yl]-1,2,4- | | | | | |
| | oxadiazol-3-yl}phenyl)propanoic acid | | | | | |
| 2 | 3-(4-{5-[8-chloro-6-(trifluoromethyl)imidazo[1,2- | A | A | nt | nt | B |
| | a]pyridin-2-yl]-1,2,4-oxadiazol-3-yl}-3- | | | | | |
| | methylphenyl)propanoic acid | | | | | |
| 3 | (2E)-3-(3-chloro-4-{5-[8-chloro-6- | A | B | nt | nt | nt |
| | (trifluoromethyl)imidazo[1,2-a]pyridin-2-yl]-1,2,4- | | | | | |
| | oxadiazol-3-yl}phenyl)prop-2-enoic acid | | | | | |
| 4 | 1-[(2,5-dichloro-4-{5-[8-chloro-6- | nt | nt | A | nt | nt |
| | (trifluoromethyl)imidazo[1,2-a]pyridin-2-yl]-1,3,4- | | | | | |
| | oxadiazol-2-yl}phenyl)oxy]propan-2-ol | | | | | |
| 5 | 4-[(2,5-dichloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}phenyl)oxy]-3-oxobutanoic acid | | | | | |
| 6 | 3-(4-{5-[8-chloro-6-(trifluoromethyl)imidazo[1,2- | A | A | nt | nt | C¹ |
| | a]pyridin-2-yl]-1,2,4-oxadiazol-3-yl}-2- | | | | | |
| | fluorophenyl)propanoic acid | | | | | |
| 7 | 3-(2-chloro-4-{5-[8-chloro-6- | A | A | nt | D | B¹ |
| | (trifluoromethyl)imidazo[1,2-a]pyridin-2-yl]-1,2,4- | | | | | |
| | oxadiazol-3-yl}phenyl)propanoic acid | | | | | |
| 8 | 3-[4-{5-[8-chloro-6-(trifluoromethyl)imidazo[1,2- | A | nt | nt | nt | nt |
| | a]pyridin-2-yl]-1,2,4-oxadiazol-3-yl}-3- | | | | | |
| | (trifluoromethyl)phenyl]propanoic acid | | | | | |
| 9 | 2-[(2,5-dichloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}phenyl)oxy]-1-(hydroxymethyl)ethyl | | | | | |
| | dihydrogen phosphate | | | | | |
| 10 | 3-(4-{5-[8-chloro-6-(trifluoromethyl)imidazo[1,2- | A | B | nt | nt | D¹ |
| | a]pyridin-2-yl]-1,2,4-oxadiazol-3-yl}-3,5- | | | | | |
| | difluorophenyl)propanoic acid | | | | | |
| 11 | 3-chloro-4-{5-[8-chloro-6-(trifluoromethyl)imidazo[1,2- | B | nt | nt | nt | nt |
| | a]pyridin-2-yl]-1,2,4-oxadiazol-3-yl}phenylalanine | | | | | |
| 12 | 8-chloro-2-[3-(2,5-dichloro-4- | nt | nt | A | nt | nt |
| | {[(methylsulfonyl)methyl]oxy}phenyl)-1,2,4-oxadiazol- | | | | | |
| | 5-yl]-6-(trifluoromethyl)imidazo[1,2-a]pyridine | | | | | |
| 13 | 1-[(2,5-dichloro-4-{5-[8-chloro-6- | nt | nt | A | nt | nt |
| | (trifluoromethyl)imidazo[1,2-a]pyridin-2-yl]-1,3,4- | | | | | |
| | thiadiazol-2-yl}phenyl)oxy]propan-2-ol | | | | | |
| 14 | (1S)-2-[(2,5-dichloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}phenyl)oxy]-1-methylethyl dihydrogen | | | | | |
| | phosphate | | | | | |
| 15 | 2-amino-3-[(2,5-dichloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}phenyl)oxy]propyl dihydrogen phosphate | | | | | |
| 16 | 2-amino-3-({5-chloro-4-[5-(8-chloroimidazo[1,2- | nt | nt | A | nt | nt |
| | a]pyridin-2-yl)-1,3,4-thiadiazol-2-yl]-2- | | | | | |
| | fluorophenyl}oxy)propan-1-ol | | | | | |
| 17 | 2-amino-3-[(5-chloro-2-fluoro-4-{5-[6- | nt | nt | A | nt | nt |
| | (trifluoromethyl)imidazo[1,2-a]pyridin-2-yl]-1,3,4- | | | | | |
| | thiadiazol-2-yl}phenyl)oxy]propan-1-ol | | | | | |
| 18 | 3-[(2,5-dichloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}phenyl)oxy]-1,1,1-trifluoropropan-2-one | | | | | |
| 19 | 3-(4-{5-[8-chloro-6-(trifluoromethyl)imidazo[1,2- | A | B | nt | nt | nt |
| | a]pyridin-2-yl]-1,2,4-oxadiazol-3-yl}-3- | | | | | |
| | fluorophenyl)propanoic acid | | | | | |
| 20 | 3-(3-chloro-4-{5-[8-chloro-6- | A | A | nt | nt | nt |
| | (trifluoromethyl)imidazo[1,2-a]pyridin-2-yl]-1,2,4- | | | | | |
| | oxadiazol-3-yl}phenyl)propanamide | | | | | |
| 21 | 3-(2,6-dichloro-4-{5-[8-chloro-6- | A | A | nt | C | nt |
| | (trifluoromethyl)imidazo[1,2-a]pyridin-2-yl]-1,2,4- | | | | | |
| | oxadiazol-3-yl}phenyl)propanoic acid | | | | | |
| 22 | 3-(4-{5-[8-chloro-6-(trifluoromethyl)imidazo[1,2- | A | nt | nt | nt | nt |
| | a]pyridin-2-yl]-1,2,4-oxadiazol-3-yl}phenyl)propanoic | | | | | |
| | acid | | | | | |
| 23 | 3-(4-{5-[8-chloro-6-(trifluoromethyl)imidazo[1,2- | A | nt | nt | nt | nt |
| | a]pyridin-2-yl]-1,2,4-oxadiazol-3-yl}-2- | | | | | |
| | methylphenyl)propanoic acid | | | | | |
| 24 | 3-(5-chloro-4-{5-[8-chloro-6- | A | A | A | A | A |
| | (trifluoromethyl)imidazo[1,2-a]pyridin-2-yl]-1,2,4- | | | | | |
| | oxadiazol-3-yl}-2-fluorophenyl)propanoic acid | | | | | |
| 25 | 3-{4-[5-(8-bromo-6-methylimidazo[1,2-a]pyridin-2-yl)- | A | A | nt | nt | nt |
| | 1,2,4-oxadiazol-3-yl]-5-chloro-2-fluorophenyl}propanoic | | | | | |
| | acid | | | | | |
| 26 | 2-[(2,5-dichloro-4-{5-[8-chloro-6- | nt | nt | B¹ | nt | nt |
| | (trifluoromethyl)imidazo[1,2-a]pyridin-2-yl]-1,2,4- | | | | | |
| | oxadiazol-3-yl}phenyl)oxy]-2-methylpropan-1-ol | | | | | |
| 27 | 3-[5-chloro-2-fluoro-4-(5-imidazo[1,2-a]pyridin-2-yl- | E¹ | nt | nt | nt | nt |
| | 1,2,4-oxadiazol-3-yl)phenyl]propanoic acid | | | | | |
| 28 | 3-{5-chloro-4-[5-(8-chloro-6-methylimidazo[1,2- | A | A | nt | nt | nt |
| | a]pyridin-2-yl)-1,2,4-oxadiazol-3-yl]-2- | | | | | |
| | fluorophenyl}propanoic acid | | | | | |
| 29 | 3-[(2,5-dichloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}phenyl)amino]propane-1,2-diol | | | | | |
| 30 | 2-[(2,5-dichloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}phenyl)amino]ethanol | | | | | |
| 31 | 3-{5-chloro-2-fluoro-4-[5-(6-iodoimidazo[1,2-a]pyridin- | A | A | nt | nt | nt |
| | 2-yl)-1,2,4-oxadiazol-3-yl]phenyl}propanoic acid | | | | | |
| 32 | 3-{5-chloro-4-[5-(8-chloroimidazo[1,2-a]pyridin-2-yl)- | B | nt | nt | nt | nt |
| | 1,2,4-oxadiazol-3-yl]-2-fluorophenyl}propanoic acid | | | | | |
| 33 | (2S)-3-[(4-{5-[8-bromo-6-(trifluoromethyl)imidazo[1,2- | nt | A | A | A | nt |
| | a]pyridin-2-yl]-1,2,4-oxadiazol-3-yl}-2,5- | | | | | |
| | dichlorophenyl)oxy]propane-1,2-diol | | | | | |
| 34 | 1-[(2,5-dichloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}phenyl)oxy]-3-hydroxypropan-2-one | | | | | |
| 35 | 3-{5-chloro-4-[5-(6,8-dichloro-7-methylimidazo[1,2- | C | nt | nt | nt | nt |
| | a]pyridin-2-yl)-1,2,4-oxadiazol-3-yl]-2- | | | | | |
| | fluorophenyl}propanoic acid | | | | | |
| 36 | 3-{5-chloro-4-[5-(8-chloro-6-nitroimidazo[1,2-a]pyridin- | A | B | nt | nt | nt |
| | 2-yl)-1,2,4-oxadiazol-3-yl]-2-fluorophenyl}propanoic | | | | | |
| | acid | | | | | |
| 37 | 8-chloro-2-{3-[3,5-dimethyl-4-(prop-2-en-1- | E¹ | nt | nt | nt | nt |
| | yloxy)phenyl]-1,2,4-oxadiazol-5-yl}-6- | | | | | |
| | (trifluoromethyl)imidazo[1,2-a]pyridine | | | | | |
| 38 | (2S)-1-[(2,5-dichloro-4-{5-[8-chloro-6- | nt | A | A | A | nt |
| | (trifluoromethyl)imidazo[1,2-a]pyridin-2-yl]-1,2,4- | | | | | |
| | oxadiazol-3-yl}phenyl)oxy]propan-2-ol | | | | | |
| 39 | (2S)-3-[(2,5-dichloro-4-{5-[8-chloro-6- | A | A | A | A | nt |
| | (trifluoromethyl)imidazo[1,2-a]pyridin-2-yl]-1,2,4- | | | | | |
| | oxadiazol-3-yl}phenyl)oxy]propane-1,2-diol | | | | | |
| 40 | 3-{5-chloro-4-[5-(6,8-difluoroimidazo[1,2-a]pyridin-2- | D¹ | nt | nt | nt | nt |
| | yl)-1,2,4-oxadiazol-3-yl]-2-fluorophenyl}propanoic acid | | | | | |
| 41 | 2-(4-{5-[8-chloro-6-(trifluoromethyl)imidazo[1,2- | B | nt | nt | nt | nt |
| | a]pyridin-2-yl]-1,2,4-oxadiazol-3-yl}-3- | | | | | |
| | fluorophenyl)cyclopropanecarboxylic acid | | | | | |
| 42 | 2-(3-chloro-4-{5-[8-chloro-6- | A | B | nt | nt | D¹ |
| | (trifluoromethyl)imidazo[1,2-a]pyridin-2-yl]-1,2,4- | | | | | |
| | oxadiazol-3-yl}phenyl)cyclopropanecarboxylic acid | | | | | |
| 43 | (2R)-2-amino-3-[(5-chloro-2-fluoro-4-{5-[6- | nt | nt | A | nt | nt |
| | (trifluoromethyl)imidazo[1,2-a]pyridin-2-yl]-1,3,4- | | | | | |
| | thiadiazol-2-yl}phenyl)oxy]propan-1-ol | | | | | |
| 44 | 2-amino-3-[(5-chloro-2-fluoro-4-{5-[6- | nt | nt | A | nt | nt |
| | (trifluoromethyl)imidazo[1,2-a]pyridin-2-yl]-1,3,4- | | | | | |
| | thiadiazol-2-yl}phenyl)oxy]-2-methylpropan-1-ol | | | | | |
| 45 | 2-amino-3-[(5-chloro-2-fluoro-4-{5-[6- | nt | nt | A | nt | nt |
| | (methyloxy)imidazo[1,2-a]pyridin-2-yl]-1,3,4-thiadiazol- | | | | | |
| | 2-yl}phenyl)oxy]propan-1-ol | | | | | |
| 46 | 2-amino-3-[(5-chloro-4-{5-[8-chloro-6- | nt | nt | A | nt | nt |
| | (trifluoromethyl)imidazo[1,2-a]pyridin-2-yl]-1,3,4- | | | | | |
| | thiadiazol-2-yl}-2-fluorophenyl)oxy]-2-methylpropan-1- | | | | | |
| | ol | | | | | |
| 47 | 3-{4-[5-(8-bromo-6-cyanoimidazo[1,2-a]pyridin-2-yl)- | A | B | nt | nt | nt |
| | 1,2,4-oxadiazol-3-yl]-5-chloro-2-fluorophenyl}propanoic | | | | | |
| | acid | | | | | |
| 48 | 3-{5-chloro-4-[5-(8-chloro-6-cyanoimidazo[1,2- | A | nt | nt | nt | nt |
| | a]pyridin-2-yl)-1,2,4-oxadiazol-3-yl]-2- | | | | | |
| | fluorophenyl}propanoic acid | | | | | |
| 49 | 3-(4-{5-[8-bromo-6-(trifluoromethyl)imidazo[1,2- | A | A | A | A | B |
| | a]pyridin-2-yl]-1,2,4-oxadiazol-3-yl}-5-chloro-2- | | | | | |
| | fluorophenyl)propanoic acid | | | | | |
| 50 | 3-[(2,5-dichloro-4-{3-[8-chloro-6- | nt | A | A | A | nt |
| | (trifluoromethyl)imidazo[1,2-a]pyridin-2-yl]-1,2,4- | | | | | |
| | oxadiazol-5-yl}phenyl)oxy]propane-1,2-diol | | | | | |
| 51 | 3-[(2,5-dichloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}phenyl)oxy]-2-oxopropyl dihydrogen | | | | | |
| | phosphate | | | | | |
| 52 | {[(2,5-dichloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}phenyl)oxy]methyl}phosphonic acid | | | | | |
| 53 | 3-[(2,5-dichloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}phenyl)oxy]-2-hydroxypropyl dihydrogen | | | | | |
| | phosphate | | | | | |
| 54 | 3-(2,5-dichloro-4-{5-[8-chloro-6- | nt | A | A | nt | nt |
| | (trifluoromethyl)imidazo[1,2-a]pyridin-2-yl]-1,2,4- | | | | | |
| | oxadiazol-3-yl}phenyl)propane-1,2-diol | | | | | |
| 55 | 3-(5-chloro-4-{5-[6-chloro-7-(methyloxy)imidazo[1,2- | E¹ | nt | nt | nt | nt |
| | a]pyridin-2-yl]-1,2,4-oxadiazol-3-yl}-2- | | | | | |
| | fluorophenyl)propanoic acid | | | | | |
| 56 | 3-(2-chloro-4-{5-[8-chloro-6- | A | A | A | A | A |
| | (trifluoromethyl)imidazo[1,2-a]pyridin-2-yl]-1,2,4- | | | | | |
| | oxadiazol-3-yl}-5-methylphenyl)propanoic acid | | | | | |
| 57 | 3-[(3-chloro-4-{5-[8-chloro-6- | A | nt | nt | nt | nt |
| | (trifluoromethyl)imidazo[1,2-a]pyridin-2-yl]-1,2,4- | | | | | |
| | oxadiazol-3-yl}phenyl)oxy]-2-hydroxypropanoic acid | | | | | |
| 58 | 3-[(2,5-dichloro-4-{5-[8-chloro-6- | nt | A | nt | nt | nt |
| | (trifluoromethyl)imidazo[1,2-a]pyridin-2-yl]-1,2,4- | | | | | |
| | oxadiazol-3-yl}phenyl)oxy]-2-hydroxypropanoic acid | | | | | |
| 59 | 3-[5-chloro-4-(5-{8-chloro-6- | D¹ | nt | nt | nt | nt |
| | [(methylsulfonyl)amino]imidazo[1,2-a]pyridin-2-yl}- | | | | | |
| | 1,2,4-oxadiazol-3-yl)-2-fluorophenyl]propanoic acid | | | | | |
| 60 | 3-(4-{5-[8-chloro-6-(trifluoromethyl)imidazo[1,2- | A | nt | nt | nt | nt |
| | a]pyridin-2-yl]-1,2,4-oxadiazol-3-yl}-2,5- | | | | | |
| | difluorophenyl)propanoic acid | | | | | |
| 61 | 3-(5-chloro-4-{5-[8-chloro-6-(methyloxy)imidazo[1,2- | A | A | nt | na | B |
| | a]pyridin-2-yl]-1,2,4-oxadiazol-3-yl}-2- | | | | | |
| | fluorophenyl)propanoic acid | | | | | |
| 62 | 3-{5-chloro-4-[5-(6,8-dibromo-5-methylimidazo[1,2- | A | nt | nt | nt | nt |
| | a]pyridin-2-yl)-1,2,4-oxadiazol-3-yl]-2- | | | | | |
| | fluorophenyl}propanoic acid | | | | | |
| 63 | (2E)-3-(5-chloro-4-{5-[8-chloro-6- | A | A | nt | nt | B |
| | (trifluoromethyl)imidazo[1,2-a]pyridin-2-yl]-1,2,4- | | | | | |
| | oxadiazol-3-yl}-2-fluorophenyl)prop-2-enoic acid | | | | | |
| 64 | 3-{4-[5-(6-bromo-8-chloroimidazo[1,2-a]pyridin-2-yl)- | A | A | nt | nt | nt |
| | 1,2,4-oxadiazol-3-yl]-5-chloro-2-fluorophenyl}propanoic | | | | | |
| | acid | | | | | |
| 65 | 2-({2-[(2,5-dichloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}phenyl)oxy]ethyl}amino)propane-1,3-diol | | | | | |
| 66 | 8-chloro-2-[5-(2,6-difluorophenyl)-1,3,4-oxadiazol-2-yl]- | C | nt | nt | nt | nt |
| | 6-(trifluoromethyl)imidazo[1,2-a]pyridine | | | | | |
| 67 | 3-[5-chloro-4-(5-{8-chloro-6-[(4- | nt | A | nt | nt | nt |
| | methylphenyl)oxy]imidazo[1,2-a]pyridin-2-yl}-1,2,4- | | | | | |
| | oxadiazol-3-yl)-2-fluorophenyl]propanoic acid | | | | | |
| 68 | 3-(4-{5-[8-bromo-6-(trifluoromethyl)imidazo[1,2- | A | A | A | A | nt |
| | a]pyridin-2-yl]-1,2,4-oxadiazol-3-yl}-2,5- | | | | | |
| | dichlorophenyl)propanoic acid | | | | | |
| 69 | 2-(4-{5-[8-chloro-6-(trifluoromethyl)imidazo[1,2- | A | B | nt | nt | D¹ |
| | a]pyridin-2-yl]-1,2,4-oxadiazol-3-yl}-3- | | | | | |
| | methylphenyl)cyclopropanecarboxylic acid | | | | | |
| 70 | 2-[4-{5-[8-chloro-6-(trifluoromethyl)imidazo[1,2- | B | nt | nt | nt | nt |
| | a]pyridin-2-yl]-1,2,4-oxadiazol-3-yl}-3- | | | | | |
| | (trifluoromethyl)phenyl]cyclopropanecarboxylic acid | | | | | |
| 71 | 3-{5-chloro-4-[5-(6,8-dibromoimidazo[1,2-a]pyridin-2- | nt | A | nt | nt | nt |
| | yl)-1,2,4-oxadiazol-3-yl]-2-fluorophenyl}propanoic acid | | | | | |
| 72 | 2-(3-chloro-4-{5-[8-chloro-6- | A | nt | nt | nt | nt |
| | (trifluoromethyl)imidazo[1,2-a]pyridin-2-yl]-1,2,4- | | | | | |
| | oxadiazol-3-yl}phenyl)cyclopropanecarboxamide | | | | | |
| 73 | 8-chloro-2-[5-(2-chlorophenyl)-1,3,4-oxadiazol-2-yl]-6- | A | nt | nt | nt | nt |
| | (trifluoromethyl)imidazo[1,2-a]pyridine | | | | | |
| 74 | 3-[3-chloro-4-({5-[8-chloro-6- | E¹ | nt | nt | nt | nt |
| | (trifluoromethyl)imidazo[1,2-a]pyridin-2-yl]-1,3,4- | | | | | |
| | oxadiazol-2-yl}amino)phenyl]propanoic acid | | | | | |
| 75 | 2-(4-{5-[8-chloro-6-(trifluoromethyl)imidazo[1,2- | C¹ | nt | nt | nt | nt |
| | a]pyridin-2-yl]-1,2,4-oxadiazol-3- | | | | | |
| | yl}phenyl)cyclopropanecarboxylic acid | | | | | |
| 76 | 8-chloro-2-[3-(2,5-dichloro-4-{[2- | nt | A | nt | nt | nt |
| | (methyloxy)ethyl]oxy}phenyl)-1,2,4-oxadiazol-5-yl]-6- | | | | | |
| | (trifluoromethyl)imidazo[1,2-a]pyridine | | | | | |
| 77 | 3-[(2,5-dichloro-4-{5-[8-chloro-6- | nt | A | A | nt | nt |
| | (trifluoromethyl)imidazo[1,2-a]pyridin-2-yl]-1,2,4- | | | | | |
| | oxadiazol-3-yl}phenyl)oxy]-1,1,1-trifluoropropan-2-ol | | | | | |
| 78 | 2-(4-{5-[8-chloro-6-(trifluoromethyl)imidazo[1,2- | B | nt | nt | nt | nt |
| | a]pyridin-2-yl]-1,2,4-oxadiazol-3-yl}-2- | | | | | |
| | fluorophenyl)cyclopropanecarboxylic acid | | | | | |
| 79 | 2-(4-{5-[8-chloro-6-(trifluoromethyl)imidazo[1,2- | A | A | nt | C | B |
| | a]pyridin-2-yl]-1,2,4-oxadiazol-3-yl}-2-fluoro-5- | | | | | |
| | methylphenyl)cyclopropanecarboxylic acid | | | | | |
| 80 | 2-amino-3-[(2,5-dichloro-4-{3-[8-chloro-6- | nt | nt | A | nt | nt |
| | (trifluoromethyl)imidazo[1,2-a]pyridin-2-yl]-1,2,4- | | | | | |
| | oxadiazol-5-yl}phenyl)oxy]propan-1-ol | | | | | |
| 81 | 2-amino-3-[(5-chloro-4-{3-[8-chloro-6- | nt | nt | B | nt | nt |
| | (trifluoromethyl)imidazo[1,2-a]pyridin-2-yl]-1,2,4- | | | | | |
| | oxadiazol-5-yl}-2-fluorophenyl)oxy]propan-1-ol | | | | | |
| 82 | 2-amino-3-[(5-chloro-4-{5-[8-chloro-6- | nt | nt | A | nt | nt |
| | (trifluoromethyl)imidazo[1,2-a]pyridin-2-yl]-1,3,4- | | | | | |
| | thiadiazol-2-yl}-2-fluorophenyl)oxy]propyl dihydrogen | | | | | |
| | phosphate | | | | | |
| 83 | 2-[(5-chloro-4-{5-[8-chloro-6- | B | nt | nt | nt | nt |
| | (trifluoromethyl)imidazo[1,2-a]pyridin-2-yl]-1,2,4- | | | | | |
| | oxadiazol-3-yl}-2-fluorophenyl)oxy]propanoic acid | | | | | |
| 84 | {(2R,4S)-4-[(3-chloro-4-{5-[8-chloro-6- | B | nt | nt | nt | nt |
| | (trifluoromethyl)imidazo[1,2-a]pyridin-2-yl]-1,2,4- | | | | | |
| | oxadiazol-3-yl}phenyl)oxy]pyrrolidin-2-yl}methanol | | | | | |
| 85 | 2-(5-chloro-4-{5-[8-chloro-6- | A | A | nt | nt | B |
| | (trifluoromethyl)imidazo[1,2-a]pyridin-2-yl]-1,2,4- | | | | | |
| | oxadiazol-3-yl}-2-fluorophenyl)cyclopropanecarboxylic | | | | | |
| | acid | | | | | |
| 86 | 2-(4-{5-[8-chloro-6-(trifluoromethyl)imidazo[1,2- | B | nt | nt | nt | nt |
| | a]pyridin-2-yl]-1,2,4-oxadiazol-3-yl}-2- | | | | | |
| | methylphenyl)cyclopropanecarboxylic acid | | | | | |
| 87 | {(2S,4S)-4-[(3-chloro-4-{5-[8-chloro-6- | A | nt | nt | nt | nt |
| | (trifluoromethyl)imidazo[1,2-a]pyridin-2-yl]-1,2,4- | | | | | |
| | oxadiazol-3-yl}phenyl)oxy]pyrrolidin-2-yl}methanol | | | | | |
| 88 | 2-(4-{5-[8-chloro-6-(trifluoromethyl)imidazo[1,2- | A | nt | nt | nt | nt |
| | a]pyridin-2-yl]-1,2,4-oxadiazol-3-yl}-2,5- | | | | | |
| | difluorophenyl)cyclopropanecarboxylic acid | | | | | |
| 89 | 2-(4-{5-[8-bromo-6-(trifluoromethyl)imidazo[1,2- | A | A | nt | C | nt |
| | a]pyridin-2-yl]-1,2,4-oxadiazol-3-yl}-5-chloro-2- | | | | | |
| | fluorophenyl)cyclopropanecarboxylic acid | | | | | |
| 90 | 3-chloro-4-{5-[8-chloro-6-(trifluoromethyl)imidazo[1,2- | A | nt | nt | nt | nt |
| | a]pyridin-2-yl]-1,2,4-oxadiazol-3-yl}aniline | | | | | |
| 91 | (2R)-2-[(4-{5-[8-chloro-6-(trifluoromethyl)imidazo[1,2- | A | nt | nt | nt | nt |
| | a]pyridin-2-yl]-1,2,4-oxadiazol-3-yl}-2,6- | | | | | |
| | dimethylphenyl)oxy]propan-1-ol | | | | | |
| 92 | {3-[(5-chloro-4-{5-[8-chloro-6- | A | A | nt | nt | nt |
| | (trifluoromethyl)imidazo[1,2-a]pyridin-2-yl]-1,2,4- | | | | | |
| | oxadiazol-3-yl}-2-fluorophenyl)oxy]pyrrolidin-1- | | | | | |
| | yl}acetic acid | | | | | |
| 93 | (2R)-2-[(5-chloro-4-{5-[8-chloro-6- | A | A | A | A | A |
| | (trifluoromethyl)imidazo[1,2-a]pyridin-2-yl]-1,2,4- | | | | | |
| | oxadiazol-3-yl}-2-fluorophenyl)oxy]propan-1-ol | | | | | |
| 94 | N-(3-chloro-4-{5-[8-chloro-6- | A | B | nt | na | C |
| | (trifluoromethyl)imidazo[1,2-a]pyridin-2-yl]-1,2,4- | | | | | |
| | oxadiazol-3-yl}phenyl)methanesulfonamide | | | | | |
| 95 | N-(3-chloro-4-{5-[8-chloro-6- | B | nt | nt | nt | nt |
| | (trifluoromethyl)imidazo[1,2-a]pyridin-2-yl]-1,2,4- | | | | | |
| | oxadiazol-3-yl}phenyl)-beta-alanine | | | | | |
| 96 | (2S)-2-[(5-chloro-4-{5-[8-chloro-6- | A | A | nt | A | B |
| | (trifluoromethyl)imidazo[1,2-a]pyridin-2-yl]-1,2,4- | | | | | |
| | oxadiazol-3-yl}-2-fluorophenyl)oxy]propan-1-ol | | | | | |
| 97 | (4S)-4-[(3-chloro-4-{5-[8-chloro-6- | A | A | nt | nt | nt |
| | (trifluoromethyl)imidazo[1,2-a]pyridin-2-yl]-1,2,4- | | | | | |
| | oxadiazol-3-yl}phenyl)oxy]-D-proline | | | | | |
| 98 | N-(4-{5-[8-chloro-6-(trifluoromethyl)imidazo[1,2- | A | A | nt | nt | nt |
| | a]pyridin-2-yl]-1,2,4-oxadiazol-3-yl}-3- | | | | | |
| | methylphenyl)methanesulfonamide | | | | | |
| 99 | 3-(3-chloro-4-{5-[8-chloro-6- | A | B | nt | nt | D¹ |
| | (trifluoromethyl)imidazo[1,2-a]pyridin-2-yl]-1,3,4- | | | | | |
| | oxadiazol-2-yl}phenyl)propanoic acid | | | | | |
| 100 | 2-amino-3-[(2,5-dichloro-4-{5-[6- | nt | nt | A | nt | nt |
| | (trifluoromethyl)imidazo[1,2-a]pyridin-2-yl]-1,3,4- | | | | | |
| | thiadiazol-2-yl}phenyl)oxy]propan-1-ol | | | | | |
| 101 | 2-amino-3-({5-chloro-4-[5-(6-chloroimidazo[1,2- | nt | nt | A | nt | nt |
| | a]pyridin-2-yl)-1,3,4-thiadiazol-2-yl]-2- | | | | | |
| | fluorophenyl}oxy)propan-1-ol | | | | | |
| 102 | 2-amino-3-({5-chloro-2-fluoro-4-[5-(6-iodoimidazo[1,2- | nt | nt | A | nt | nt |
| | a]pyridin-2-yl)-1,3,4-thiadiazol-2-yl]phenyl}oxy)propan- | | | | | |
| | 1-ol | | | | | |
| 103 | N-(3-chloro-4-{5-[8-chloro-6- | B | nt | nt | nt | nt |
| | (trifluoromethyl)imidazo[1,2-a]pyridin-2-yl]-1,2,4- | | | | | |
| | oxadiazol-3-yl}phenyl)glycine | | | | | |
| 104 | 1-[(2,6-dichloro-4-{5-[8-chloro-6- | nt | A | nt | nt | nt |
| | (trifluoromethyl)imidazo[1,2-a]pyridin-2-yl]-1,2,4- | | | | | |
| | oxadiazol-3-yl}phenyl)oxy]propan-2-ol | | | | | |
| 105 | 3-[(2,5-dichloro-4-{5-[8-chloro-6- | nt | A | A | A | nt |
| | (trifluoromethyl)imidazo[1,2-a]pyridin-2-yl]-1,2,4- | | | | | |
| | oxadiazol-3-yl}phenyl)oxy]propan-1-ol | | | | | |
| 106 | N-(4-{5-[8-chloro-6-(trifluoromethyl)imidazo[1,2- | A | nt | nt | nt | nt |
| | a]pyridin-2-yl]-1,2,4-oxadiazol-3-yl}-2-fluoro-5- | | | | | |
| | methylphenyl)methanesulfonamide | | | | | |
| 107 | (2S)-2-[(4-{5-[8-chloro-6-(trifluoromethyl)imidazo[1,2- | A | nt | nt | nt | nt |
| | a]pyridin-2-yl]-1,2,4-oxadiazol-3-yl}-2,6- | | | | | |
| | dimethylphenyl)oxy]propan-1-ol | | | | | |
| 108 | N-(3-chloro-4-{5-[8-chloro-6- | D¹ | nt | nt | nt | nt |
| | (trifluoromethyl)imidazo[1,2-a]pyridin-2-yl]-1,2,4- | | | | | |
| | oxadiazol-3-yl}phenyl)-2- | | | | | |
| | (diethylamino)ethanesulfonamide | | | | | |
| 109 | N-(4-{5-[8-chloro-6-(trifluoromethyl)imidazo[1,2- | B | nt | nt | nt | nt |
| | a]pyridin-2-yl]-1,2,4-oxadiazol-3-yl}-2- | | | | | |
| | fluorophenyl)methanesulfonamide | | | | | |
| 110 | 1-[(4-{5-[8-bromo-6-(trifluoromethyl)imidazo[1,2- | nt | A | nt | nt | nt |
| | a]pyridin-2-yl]-1,2,4-oxadiazol-3-yl}-2,5- | | | | | |
| | dichlorophenyl)oxy]-3-fluoropropan-2-ol | | | | | |
| 111 | N-(5-chloro-4-{5-[8-chloro-6- | A | nt | nt | nt | nt |
| | (trifluoromethyl)imidazo[1,2-a]pyridin-2-yl]-1,2,4- | | | | | |
| | oxadiazol-3-yl}-2-fluorophenyl)methanesulfonamide | | | | | |
| 112 | {3-[(3-chloro-4-{5-[8-chloro-6- | A | nt | nt | nt | nt |
| | (trifluoromethyl)imidazo[1,2-a]pyridin-2-yl]-1,2,4- | | | | | |
| | oxadiazol-3-yl}phenyl)oxy]pyrrolidin-1-yl}acetic acid | | | | | |
| 113 | ethyl 2-[(5-chloro-4-{5-[8-chloro-6- | A | nt | nt | nt | nt |
| | (trifluoromethyl)imidazo[1,2-a]pyridin-2-yl]-1,2,4- | | | | | |
| | oxadiazol-3-yl}-2-fluorophenyl)oxy]propanoate | | | | | |
| 114 | N-{4-[5-(8-bromo-6-cyanoimidazo[1,2-a]pyridin-2-yl)- | D¹ | nt | nt | nt | nt |
| | 1,2,4-oxadiazol-3-yl]-3- | | | | | |
| | chlorophenyl}methanesulfonamide | | | | | |
| 115 | 2-[(3-chloro-4-{5-[8-chloro-6- | nt | A | nt | nt | nt |
| | (trifluoromethyl)imidazo[1,2-a]pyridin-2-yl]-1,2,4- | | | | | |
| | oxadiazol-3-yl}phenyl)amino]ethanol | | | | | |
| 116 | 1-[(4-{5-[8-chloro-6-(trifluoromethyl)imidazo[1,2- | B | nt | nt | nt | nt |
| | a]pyridin-2-yl]-1,2,4-oxadiazol-3- | | | | | |
| | yl}phenyl)methyl]azetidine-3-carboxylic acid | | | | | |
| 117 | N-(4-{5-[8-chloro-6-(trifluoromethyl)imidazo[1,2- | A | nt | nt | nt | nt |
| | a]pyridin-2-yl]-1,2,4-oxadiazol-3- | | | | | |
| | yl}phenyl)methanesulfonamide | | | | | |
| 118 | 1-[(2,5-dichloro-4-{5-[8-chloro-6- | nt | A | A | A | nt |
| | (trifluoromethyl)imidazo[1,2-a]pyridin-2-yl]-1,2,4- | | | | | |
| | oxadiazol-3-yl}phenyl)oxy]-3-fluoropropan-2-ol | | | | | |
| 119 | N-(4-{5-[8-chloro-6-(trifluoromethyl)imidazo[1,2- | A | nt | nt | nt | nt |
| | a]pyridin-2-yl]-1,2,4-oxadiazol-3-yl}-2- | | | | | |
| | methylphenyl)methanesulfonamide | | | | | |
| 120 | N-[(3-chloro-4-{5-[8-chloro-6- | C | nt | nt | nt | nt |
| | (trifluoromethyl)imidazo[1,2-a]pyridin-2-yl]-1,2,4- | | | | | |
| | oxadiazol-3-yl}phenyl)methyl]glycine | | | | | |
| 121 | 1-[(3-{5-[8-chloro-6-(trifluoromethyl)imidazo[1,2- | C | nt | nt | nt | nt |
| | a]pyridin-2-yl]-1,2,4-oxadiazol-3- | | | | | |
| | yl}phenyl)methyl]azetidine-3-carboxylic acid | | | | | |
| 122 | N-(2-chloro-4-{5-[8-chloro-6- | A | nt | nt | nt | nt |
| | (trifluoromethyl)imidazo[1,2-a]pyridin-2-yl]-1,2,4- | | | | | |
| | oxadiazol-3-yl}phenyl)methanesulfonamide | | | | | |
| 123 | N-[(4-{5-[8-chloro-6-(trifluoromethyl)imidazo[1,2- | B | nt | nt | nt | nt |
| | a]pyridin-2-yl]-1,2,4-oxadiazol-3-yl}phenyl)methyl]- | | | | | |
| | beta-alanine | | | | | |
| 124 | 1-[(3-chloro-4-{5-[8-chloro-6- | B | B | nt | na | D¹ |
| | (trifluoromethyl)imidazo[1,2-a]pyridin-2-yl]-1,2,4- | | | | | |
| | oxadiazol-3-yl}phenyl)methyl]azetidine-3-carboxylic | | | | | |
| | acid | | | | | |
| 125 | N-{4-[5-(8-bromo-6-methylimidazo[1,2-a]pyridin-2-yl)- | D¹ | nt | nt | nt | nt |
| | 1,2,4-oxadiazol-3-yl]-3- | | | | | |
| | chlorophenyl}methanesulfonamide | | | | | |
| 126 | 2-(3-chloro-4-{5-[8-chloro-6- | B | nt | nt | nt | nt |
| | (trifluoromethyl)imidazo[1,2-a]pyridin-2-yl]-1,2,4- | | | | | |
| | oxadiazol-3-yl}phenyl)propan-2-ol | | | | | |
| 127 | 1-[(2-chloro-4-{5-[8-chloro-6- | B | nt | nt | nt | nt |
| | (trifluoromethyl)imidazo[1,2-a]pyridin-2-yl]-1,2,4- | | | | | |
| | oxadiazol-3-yl}phenyl)methyl]azetidine-3-carboxylic | | | | | |
| | acid | | | | | |
| 128 | N-(3-chloro-4-{5-[8-chloro-6- | B | nt | nt | nt | nt |
| | (trifluoromethyl)imidazo[1,2-a]pyridin-2-yl]-1,2,4- | | | | | |
| | oxadiazol-3-yl}phenyl)ethenesulfonamide | | | | | |
| 129 | N-(4-{5-[8-chloro-6-(trifluoromethyl)imidazo[1,2- | B | nt | nt | nt | nt |
| | a]pyridin-2-yl]-1,2,4-oxadiazol-3-yl}-3- | | | | | |
| | fluorophenyl)methanesulfonamide | | | | | |
| 130 | N-[(3-chloro-4-{5-[8-chloro-6- | A | B | nt | nt | C |
| | (trifluoromethyl)imidazo[1,2-a]pyridin-2-yl]-1,2,4- | | | | | |
| | oxadiazol-3-yl}phenyl)methyl]-beta-alanine | | | | | |
| 131 | N-[(4-{5-[8-bromo-6-(trifluoromethyl)imidazo[1,2- | A | B | nt | nt | D |
| | a]pyridin-2-yl]-1,2,4-oxadiazol-3-yl}-3- | | | | | |
| | chlorophenyl)methyl]-beta-alanine | | | | | |
| 132 | 1-[(4-{5-[8-bromo-6-(trifluoromethyl)imidazo[1,2- | A | B | nt | na | D |
| | a]pyridin-2-yl]-1,2,4-oxadiazol-3-yl}-3- | | | | | |
| | chlorophenyl)methyl]azetidine-3-carboxylic acid | | | | | |
| 133 | (3-chloro-4-{5-[8-chloro-6-(trifluoromethyl)imidazo[1,2- | D¹ | nt | nt | nt | nt |
| | a]pyridin-2-yl]-1,2,4-oxadiazol-3-yl}phenyl)acetic acid | | | | | |
| 134 | 3-(3-chloro-4-{5-[8-chloro-6- | C | nt | nt | nt | nt |
| | (trifluoromethyl)imidazo[1,2-a]pyridin-2-yl]-1,2,4- | | | | | |
| | oxadiazol-3-yl}phenyl)-1,2,4-oxadiazol-5(2H)-one | | | | | |
| 135 | 4-amino-3-(3-chloro-4-{5-[8-chloro-6- | B | nt | nt | nt | nt |
| | (trifluoromethyl)imidazo[1,2-a]pyridin-2-yl]-1,2,4- | | | | | |
| | oxadiazol-3-yl}phenyl)-4-oxobutanoic acid | | | | | |
| 136 | N-[(3-chloro-4-{5-[8-chloro-6- | A | A | nt | nt | nt |
| | (trifluoromethyl)imidazo[1,2-a]pyridin-2-yl]-1,2,4- | | | | | |
| | oxadiazol-3-yl}phenyl)methyl]methanesulfonamide | | | | | |
| 137 | 3-(3-chloro-4-{5-[8-chloro-6- | D¹ | nt | nt | nt | nt |
| | (trifluoromethyl)imidazo[1,2-a]pyridin-2-yl]-1,2,4- | | | | | |
| | oxadiazol-3-yl}phenyl)-3-(ethylamino)propanoic acid | | | | | |
| 138 | 2-{[(2,5-dichloro-4-{5-[8-chloro-6- | nt | B¹ | nt | nt | nt |
| | (trifluoromethyl)imidazo[1,2-a]pyridin-2-yl]-1,2,4- | | | | | |
| | oxadiazol-3-yl}phenyl)methyl]amino}ethanol | | | | | |
| 139 | 3-(3-chloro-4-{5-[8-chloro-6- | A | A | nt | nt | B¹ |
| | (trifluoromethyl)imidazo[1,2-a]pyridin-2-yl]-1,2,4- | | | | | |
| | oxadiazol-3-yl}phenyl)-3-hydroxypropanoic acid | | | | | |
| 140 | 8-chloro-2-(3-{2-chloro-4-[2- | A | nt | nt | nt | nt |
| | (methylsulfonyl)ethyl]phenyl}-1,2,4-oxadiazol-5-yl)-6- | | | | | |
| | (trifluoromethyl)imidazo[1,2-a]pyridine | | | | | |
| 141 | 8-chloro-2-[3-(2,6-difluorophenyl)-1,2,4-oxadiazol-5-yl]- | C | B | nt | nt | nt |
| | 6-(trifluoromethyl)imidazo[1,2-a]pyridine | | | | | |
| 142 | 8-chloro-2-[3-(2-fluorophenyl)-1,2,4-oxadiazol-5-yl]-6- | B | C | nt | nt | nt |
| | (trifluoromethyl)imidazo[1,2-a]pyridine | | | | | |
| 143 | 8-chloro-6-(trifluoromethyl)-2-{3-[3- | C | nt | nt | nt | nt |
| | (trifluoromethyl)phenyl]-1,2,4-oxadiazol-5- | | | | | |
| | yl}imidazo[1,2-a]pyridine | | | | | |
| 144 | 2-{3-[(5-chloro-4-{5-[8-chloro-6- | B | nt | nt | nt | nt |
| | (trifluoromethyl)imidazo[1,2-a]pyridin-2-yl]-1,2,4- | | | | | |
| | oxadiazol-3-yl}-2-fluorophenyl)oxy]pyrrolidin-1- | | | | | |
| | yl}ethanol | | | | | |
| 145 | 2-[(2,5-dichloro-4-{5-[8-chloro-6- | A | A | A | nt | nt |
| | (trifluoromethyl)imidazo[1,2-a]pyridin-2-yl]-1,2,4- | | | | | |
| | oxadiazol-3-yl}phenyl)oxy]propan-1-ol | | | | | |
| 146 | 8-chloro-2-[3-(2,4-difluorophenyl)-1,2,4-oxadiazol-5-yl]- | C | nt | nt | nt | nt |
| | 6-(trifluoromethyl)imidazo[1,2-a]pyridine | | | | | |
| 147 | 8-bromo-2-[3-(2-chloro-6-fluorophenyl)-1,2,4-oxadiazol- | B | nt | nt | nt | nt |
| | 5-yl]-6-(trifluoromethyl)imidazo[1,2-a]pyridine | | | | | |
| 148 | 3-(5-chloro-4-{5-[8-chloro-6- | A | nt | nt | nt | nt |
| | (methylsulfonyl)imidazo[1,2-a]pyridin-2-yl]-1,2,4- | | | | | |
| | oxadiazol-3-yl}-2-fluorophenyl)propanoic acid | | | | | |
| 149 | 3-(5-chloro-4-{5-[8-chloro-6-(2- | A | A | nt | nt | A¹ |
| | methylpropyl)imidazo[1,2-a]pyridin-2-yl]-1,2,4- | | | | | |
| | oxadiazol-3-yl}-2-fluorophenyl)propanoic acid | | | | | |
| 150 | 8-chloro-2-[3-(2-methylphenyl)-1,2,4-oxadiazol-5-yl]-6- | A | B | nt | nt | nt |
| | (trifluoromethyl)imidazo[1,2-a]pyridine | | | | | |
| 151 | 8-chloro-2-[3-(1H-indol-5-yl)-1,2,4-oxadiazol-5-yl]-6- | B | nt | nt | nt | nt |
| | (trifluoromethyl)imidazo[1,2-a]pyridine | | | | | |
| 152 | 8-chloro-2-[3-(4-fluoro-2-methylphenyl)-1,2,4-oxadiazol- | A | nt | nt | nt | nt |
| | 5-yl]-6-(trifluoromethyl)imidazo[1,2-a]pyridine | | | | | |
| 153 | 3-(4-{5-[8-bromo-6-(trifluoromethyl)imidazo[1,2- | A | B | nt | nt | C¹ |
| | a]pyridin-2-yl]-1,2,4-oxadiazol-3-yl}-3- | | | | | |
| | methylphenyl)butanoic acid | | | | | |
| 154 | 2-amino-2-[2-(5-chloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}-2-fluorophenyl)ethyl]propane-1,3-diol | | | | | |
| 155 | (2R)-2-[(2,5-dichloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}phenyl)oxy]propan-1-ol | | | | | |
| 156 | (2S)-2-[(2,5-dichloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}phenyl)oxy]propan-1-ol | | | | | |
| 157 | 8-chloro-2-[3-(2-chloro-3-fluorophenyl)-1,2,4-oxadiazol- | B | nt | nt | nt | nt |
| | 5-yl]-6-(trifluoromethyl)imidazo[1,2-a]pyridine | | | | | |
| 158 | 5-{5-[8-chloro-6-(trifluoromethyl)imidazo[1,2-a]pyridin- | D¹ | nt | nt | nt | nt |
| | 2-yl]-1,2,4-oxadiazol-3-yl}-1-benzofuran-2-carboxylic | | | | | |
| | acid | | | | | |
| 159 | 8-bromo-2-[3-(2-chlorophenyl)-1,2,4-oxadiazol-5-yl]-6- | A | B | nt | nt | nt |
| | (trifluoromethyl)imidazo[1,2-a]pyridine | | | | | |
| 160 | 3-{5-chloro-4-[5-(8-cyano-6-methylimidazo[1,2- | A | B | nt | nt | nt |
| | a]pyridin-2-yl)-1,2,4-oxadiazol-3-yl]-2- | | | | | |
| | fluorophenyl}propanoic acid | | | | | |
| 161 | 3-[(4-{5-[8-chloro-6-(trifluoromethyl)imidazo[1,2- | A | A | nt | A | B |
| | a]pyridin-2-yl]-1,2,4-oxadiazol-3-yl}-2,6- | | | | | |
| | dimethylphenyl)oxy]propane-1,2-diol | | | | | |
| 162 | 8-chloro-2-[3-(1H-indol-4-yl)-1,2,4-oxadiazol-5-yl]-6- | A | nt | nt | nt | nt |
| | (trifluoromethyl)imidazo[1,2-a]pyridine | | | | | |
| 163 | 8-chloro-2-[3-(2-chloro-6-fluorophenyl)-1,2,4-oxadiazol- | B | nt | nt | nt | nt |
| | 5-yl]-6-(trifluoromethyl)imidazo[1,2-a]pyridine | | | | | |
| 164 | 5-{5-[8-chloro-6-(trifluoromethyl)imidazo[1,2-a]pyridin- | B | nt | nt | nt | nt |
| | 2-yl]-1,2,4-oxadiazol-3-yl}-1H-benzimidazole | | | | | |
| 165 | 8-chloro-6-(trifluoromethyl)-2-{3-[2- | B | nt | nt | nt | nt |
| | (trifluoromethyl)phenyl]-1,2,4-oxadiazol-5- | | | | | |
| | yl}imidazo[1,2-a]pyridine | | | | | |
| 166 | 1-[(2,5-dichloro-4-{5-[8-chloro-6- | nt | A | A | B | nt |
| | (trifluoromethyl)imidazo[1,2-a]pyridin-2-yl]-1,2,4- | | | | | |
| | oxadiazol-3-yl}phenyl)oxy]-2-methylpropan-2-ol | | | | | |
| 167 | 3-(2,5-dichloro-4-{5-[8-chloro-6- | nt | A | A | A | nt |
| | (trifluoromethyl)imidazo[1,2-a]pyridin-2-yl]-1,2,4- | | | | | |
| | oxadiazol-3-yl}phenyl)propan-1-ol | | | | | |
| 168 | 2-[3-(2-bromo-4-fluorophenyl)-1,2,4-oxadiazol-5-yl]-8- | A | nt | nt | nt | nt |
| | chloro-6-(trifluoromethyl)imidazo[1,2-a]pyridine | | | | | |
| 169 | {5-[8-chloro-6-(trifluoromethyl)imidazo[1,2-a]pyridin- | E¹ | nt | nt | nt | nt |
| | 2-yl]-1,2,4-oxadiazol-3-yl}-1H-indole-2-carboxylic acid | | | | | |
| 170 | 3-(3-chloro-4-{5-[8-chloro-6- | A | nt | nt | nt | nt |
| | (trifluoromethyl)imidazo[1,2-a]pyridin-2-yl]-1,2,4- | | | | | |
| | oxadiazol-3-yl}phenyl)butanamide | | | | | |
| 171 | 3-(3-chloro-4-{5-[8-chloro-6- | A | B | nt | nt | nt |
| | (trifluoromethyl)imidazo[1,2-a]pyridin-2-yl]-1,2,4- | | | | | |
| | oxadiazol-3-yl}phenyl)-2-methylpropanoic acid | | | | | |
| 172 | 3-(3-chloro-4-{5-[8-chloro-6- | A | B | nt | na | D |
| | (trifluoromethyl)imidazo[1,2-a]pyridin-2-yl]-1,2,4- | | | | | |
| | oxadiazol-3-yl}phenyl)butanoic acid | | | | | |
| 173 | 3-(4-{5-[8-bromo-6-(trifluoromethyl)imidazo[1,2- | A | B | nt | nt | nt |
| | a]pyridin-2-yl]-1,2,4-oxadiazol-3-yl}-3-methylphenyl)-2- | | | | | |
| | methylpropanoic acid | | | | | |
| 174 | 2-(4-{5-[8-chloro-6-(trifluoromethyl)imidazo[1,2- | A | nt | nt | nt | nt |
| | a]pyridin-2-yl]-1,2,4-oxadiazol-3-yl}-1H-indol-1- | | | | | |
| | yl)acetamide | | | | | |
| 175 | 2-(4-{5-[8-chloro-6-(trifluoromethyl)imidazo[1,2- | A | nt | nt | nt | nt |
| | a]pyridin-2-yl]-1,2,4-oxadiazol-3-yl}-1H-indol-1-yl)-N- | | | | | |
| | (2-hydroxyethyl)acetamide | | | | | |
| 176 | 3-(5-chloro-4-{5-[8-chloro-6- | A | A | A | A | A |
| | (trifluoromethyl)imidazo[1,2-a]pyridin-2-yl]-1,3,4- | | | | | |
| | thiadiazol-2-yl}-2-fluorophenyl)propanoic acid | | | | | |
| 177 | 3-(4-{5-[8-bromo-6-(trifluoromethyl)imidazo[1,2- | A | A | A | A | A |
| | a]pyridin-2-yl]-1,3,4-thiadiazol-2-yl}-5-chloro-2- | | | | | |
| | fluorophenyl)propanoic acid | | | | | |
| 178 | 1-[(2,5-dichloro-4-{3-[8-chloro-6- | nt | nt | A | nt | nt |
| | (trifluoromethyl)imidazo[1,2-a]pyridin-2-yl]-1,2,4- | | | | | |
| | oxadiazol-5-yl}phenyl)oxy]propan-2-ol | | | | | |
| 179 | 1-[(5-chloro-4-{5-[8-chloro-6- | nt | nt | B | nt | nt |
| | (trifluoromethyl)imidazo[1,2-a]pyridin-2-yl]-1,3,4- | | | | | |
| | oxadiazol-2-yl}-2-fluorophenyl)oxy]propan-2-ol | | | | | |
| 180 | 3-(4-{5-[8-chloro-6-(trifluoromethyl)imidazo[1,2- | A | B | nt | nt | nt |
| | a]pyridin-2-yl]-1,2,4-oxadiazol-3-yl}-3-methylphenyl)-2- | | | | | |
| | methylpropanoic acid | | | | | |
| 181 | 3-(4-{5-[8-chloro-6-(trifluoromethyl)imidazo[1,2- | A | B | nt | nt | nt |
| | a]pyridin-2-yl]-1,2,4-oxadiazol-3-yl}-3- | | | | | |
| | methylphenyl)butanoic acid | | | | | |
| 182 | 1-[(2,5-dichloro-4-{5-[8-chloro-6- | nt | A | A | nt | nt |
| | (trifluoromethyl)imidazo[1,2-a]pyridin-2-yl]-1,2,4- | | | | | |
| | oxadiazol-3-yl}phenyl)oxy]propan-2-one | | | | | |
| 183 | 8-chloro-2-{3-[2-chloro-4-(trifluoromethyl)phenyl]- | C | nt | nt | nt | nt |
| | 1,2,4-oxadiazol-5-yl}-6-(trifluoromethyl)imidazo[1,2- | | | | | |
| | a]pyridine | | | | | |
| 184 | 1-[(5-chloro-4-{3-[8-chloro-6- | nt | nt | A | nt | nt |
| | (trifluoromethyl)imidazo[1,2-a]pyridin-2-yl]-1,2,4- | | | | | |
| | oxadiazol-5-yl}-2-fluorophenyl)oxy]propan-2-ol | | | | | |
| 185 | 4-(2,5-dichloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}phenyl)butan-2-ol | | | | | |
| 186 | 3-(4-{5-[8-chloro-6-(trifluoromethyl)imidazo[1,2- | B | nt | nt | nt | nt |
| | a]pyridin-2-yl]-1,2,4-oxadiazol-3-yl}-3- | | | | | |
| | fluorophenyl)butanoic acid | | | | | |
| 187 | 3-chloro-4-{5-[8-chloro-6-(trifluoromethyl)imidazo[1,2- | A | nt | nt | nt | nt |
| | a]pyridin-2-yl]-1,2,4-oxadiazol-3-yl}phenol | | | | | |
| 188 | O-(3-chloro-4-{5-[8-chloro-6- | B | nt | nt | nt | nt |
| | (trifluoromethyl)imidazo[1,2-a]pyridin-2-yl]-1,2,4- | | | | | |
| | oxadiazol-3-yl}phenyl)serine | | | | | |
| 189 | 3-(4-{5-[8-bromo-6-(trifluoromethyl)imidazo[1,2- | A | nt | nt | nt | nt |
| | a]pyridin-2-yl]-1,2,4-oxadiazol-3-yl}-3-chlorophenyl)-2- | | | | | |
| | methylpropanoic acid | | | | | |
| 190 | 3-(2,5-dichloro-4-{5-[8-chloro-6- | nt | B¹ | nt | nt | nt |
| | (trifluoromethyl)imidazo[1,2-a]pyridin-2-yl]-1,2,4- | | | | | |
| | oxadiazol-3-yl}phenyl)-2-methylpropanoic acid | | | | | |
| 191 | 2,5-dichloro-4-{5-[8-chloro-6- | A | A | nt | nt | nt |
| | (trifluoromethyl)imidazo[1,2-a]pyridin-2-yl]-1,2,4- | | | | | |
| | oxadiazol-3-yl}phenol | | | | | |
| 192 | 3-(4-{5-[8-chloro-6-(trifluoromethyl)imidazo[1,2- | B | nt | nt | nt | nt |
| | a]pyridin-2-yl]-1,2,4-oxadiazol-3-yl}-3-fluorophenyl)-2- | | | | | |
| | methylpropanoic acid | | | | | |
| 193 | 3-amino-3-(3-chloro-4-{5-[8-chloro-6- | B | nt | nt | nt | nt |
| | (trifluoromethyl)imidazo[1,2-a]pyridin-2-yl]-1,2,4- | | | | | |
| | oxadiazol-3-yl}phenyl)propanoic acid | | | | | |
| 194 | 3-[(3-chloro-4-{5-[8-chloro-6- | A | A | nt | C | nt |
| | (trifluoromethyl)imidazo[1,2-a]pyridin-2-yl]-1,2,4- | | | | | |
| | oxadiazol-3-yl}phenyl)oxy]propane-1,2-diol | | | | | |
| 195 | 2-[(3-chloro-4-{3-[8-chloro-6- | B | nt | nt | nt | nt |
| | (trifluoromethyl)imidazo[1,2-a]pyridin-2-yl]-1,2,4- | | | | | |
| | oxadiazol-5-yl}phenyl)oxy]ethanamine | | | | | |
| 196 | 3-(4-{5-[8-bromo-6-(trifluoromethyl)imidazo[1,2- | A | A | A | A | nt |
| | a]pyridin-2-yl]-1,2,4-oxadiazol-3-yl}-2-chloro-5- | | | | | |
| | methylphenyl)propanoic acid | | | | | |
| 197 | 4-{5-[8-bromo-6-(trifluoromethyl)imidazo[1,2-a]pyridin- | B | nt | nt | nt | nt |
| | 2-yl]-1,2,4-oxadiazol-3-yl}-2,5-dichlorophenol | | | | | |
| 198 | 3-[(5-chloro-4-{5-[8-chloro-6- | A | A | nt | A | nt |
| | (trifluoromethyl)imidazo[1,2-a]pyridin-2-yl]-1,2,4- | | | | | |
| | oxadiazol-3-yl}-2-fluorophenyl)oxy]propane-1,2-diol | | | | | |
| 199 | 3-[(3-chloro-4-{5-[8-chloro-6- | B | nt | nt | nt | nt |
| | (trifluoromethyl)imidazo[1,2-a]pyridin-2-yl]-1,2,4- | | | | | |
| | oxadiazol-3-yl}phenyl)oxy]propan-1-ol | | | | | |
| 200 | 1-[(3-chloro-4-{5-[8-chloro-6- | B | nt | nt | nt | nt |
| | (trifluoromethyl)imidazo[1,2-a]pyridin-2-yl]-1,2,4- | | | | | |
| | oxadiazol-3-yl}phenyl)oxy]propan-2-amine | | | | | |
| 201 | (2S)-2-amino-3-[(2,5-dichloro-4-{5-[8-chloro-6- | nt | nt | A | nt | nt |
| | (trifluoromethyl)imidazo[1,2-a]pyridin-2-yl]-1,3,4- | | | | | |
| | thiadiazol-2-yl}phenyl)oxy]propan-1-ol | | | | | |
| 202 | 2-amino-3-[(2,5-dichloro-4-{5-[8-chloro-6- | nt | nt | B¹ | nt | nt |
| | (trifluoromethyl)imidazo[1,2-a]pyridin-2-yl]-1,3,4- | | | | | |
| | oxadiazol-2-yl}phenyl)oxy]propan-1-ol | | | | | |
| 203 | 3-(5-chloro-4-{5-[8-chloro-6- | A | B | nt | nt | nt |
| | (trifluoromethyl)imidazo[1,2-a]pyridin-2-yl]-1,2,4- | | | | | |
| | oxadiazol-3-yl}-2-fluorophenyl)-2-methylpropanoic acid | | | | | |
| 204 | 3-({2,5-dichloro-4-[5-(8-chloro-6-iodoimidazo[1,2- | nt | A | nt | B | nt |
| | a]pyridin-2-yl)-1,2,4-oxadiazol-3- | | | | | |
| | yl]phenyl}oxy)propane-1,2-diol | | | | | |
| 205 | 2-[(5-chloro-4-{3-[8-chloro-6- | B¹ | nt | nt | nt | nt |
| | (trifluoromethyl)imidazo[1,2-a]pyridin-2-yl]-1,2,4- | | | | | |
| | oxadiazol-5-yl}-2-fluorophenyl)oxy]propanoic acid | | | | | |
| 206 | 3-(2,6-dichloro-4-{5-[8-chloro-6- | nt | A | nt | D | nt |
| | (trifluoromethyl)imidazo[1,2-a]pyridin-2-yl]-1,3,4- | | | | | |
| | thiadiazol-2-yl}phenyl)propanoic acid | | | | | |
| 207 | 3-{5-chloro-4-[5-(8-chloro-6-iodoimidazo[1,2-a]pyridin- | nt | A | nt | D | nt |
| | 2-yl)-1,3,4-thiadiazol-2-yl]-2-fluorophenyl}propanoic | | | | | |
| | acid | | | | | |
| 208 | 3-[(2,5-dichloro-4-{5-[8-chloro-6- | A | A | A | A | B |
| | (trifluoromethyl)imidazo[1,2-a]pyridin-2-yl]-1,2,4- | | | | | |
| | oxadiazol-3-yl}phenyl)oxy]propane-1,2-diol | | | | | |
| 209 | 3-[(2,6-dichloro-4-{5-[8-chloro-6- | nt | A | nt | A | nt |
| | (trifluoromethyl)imidazo[1,2-a]pyridin-2-yl]-1,2,4- | | | | | |
| | oxadiazol-3-yl}phenyl)oxy]propane-1,2-diol | | | | | |
| 210 | 2-{3-[(3-chloro-4-{5-[8-chloro-6- | D¹ | nt | nt | nt | nt |
| | (trifluoromethyl)imidazo[1,2-a]pyridin-2-yl]-1,2,4- | | | | | |
| | oxadiazol-3-yl}phenyl)oxy]pyrrolidin-1-yl}ethanol | | | | | |
| 211 | 2-(2,5-dichloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}phenyl)cyclopropanecarboxylic acid | | | | | |
| 212 | 3-[(4-{5-[8-bromo-6-(trifluoromethyl)imidazo[1,2- | nt | A | A | A | nt |
| | a]pyridin-2-yl]-1,2,4-oxadiazol-3-yl}-2,5- | | | | | |
| | dichlorophenyl)oxy]propane-1,2-diol | | | | | |
| 213 | 2-amino-3-[(3-chloro-4-{5-[8-chloro-6- | A | A | A | nt | nt |
| | (trifluoromethyl)imidazo[1,2-a]pyridin-2-yl]-1,2,4- | | | | | |
| | oxadiazol-3-yl}phenyl)oxy]propan-1-ol | | | | | |
| 214 | [2-(3-chloro-4-{5-[8-chloro-6- | A | A | nt | nt | nt |
| | (trifluoromethyl)imidazo[1,2-a]pyridin-2-yl]-1,2,4- | | | | | |
| | oxadiazol-3-yl}phenyl)cyclopropyl]methanol | | | | | |
| 215 | 2-(2-chloro-4-{5-[8-chloro-6- | A | nt | nt | nt | nt |
| | (trifluoromethyl)imidazo[1,2-a]pyridin-2-yl]-1,2,4- | | | | | |
| | oxadiazol-3-yl}phenyl)cyclopropanecarboxylic acid | | | | | |
| 216 | 1-amino-3-[(5-chloro-4-{5-[8-chloro-6- | A | A | nt | nt | nt |
| | (trifluoromethyl)imidazo[1,2-a]pyridin-2-yl]-1,2,4- | | | | | |
| | oxadiazol-3-yl}-2-fluorophenyl)oxy]propan-2-ol | | | | | |
| 217 | 2-[(5-chloro-4-{3-[8-chloro-6- | A | nt | nt | nt | nt |
| | (trifluoromethyl)imidazo[1,2-a]pyridin-2-yl]-1,2,4- | | | | | |
| | oxadiazol-5-yl}-2-fluorophenyl)oxy]propan-1-ol | | | | | |
| 218 | N-(3-chloro-4-{5-[8-chloro-6- | B | nt | nt | nt | nt |
| | (trifluoromethyl)imidazo[1,2-a]pyridin-2-yl]-1,3,4- | | | | | |
| | oxadiazol-2-yl}phenyl)methanesulfonamide | | | | | |
| 219 | 3-(3-chloro-4-{5-[8-chloro-6- | A | nt | nt | nt | nt |
| | (trifluoromethyl)imidazo[1,2-a]pyridin-2-yl]-1,3,4- | | | | | |
| | oxadiazol-2-yl}phenyl)butanoic acid | | | | | |
| 220 | 2-amino-3-[(2,5-dichloro-4-{5-[8-chloro-6- | nt | nt | A | nt | nt |
| | (trifluoromethyl)imidazo[1,2-a]pyridin-2-yl]-1,3,4- | | | | | |
| | thiadiazol-2-yl}phenyl)oxy]propan-1-ol | | | | | |
| 221 | 3-(2,5-dichloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}phenyl)-1-methylpropyl dihydrogen | | | | | |
| | phosphate | | | | | |
| 222 | 2-amino-3-[(5-chloro-4-{5-[8-chloro-6- | nt | nt | A | nt | nt |
| | (trifluoromethyl)imidazo[1,2-a]pyridin-2-yl]-1,3,4- | | | | | |
| | oxadiazol-2-yl}-2-fluorophenyl)oxy]propan-1-ol | | | | | |
| 223 | 1-amino-3-[(3-chloro-4-{5-[8-chloro-6- | A | nt | nt | nt | nt |
| | (trifluoromethyl)imidazo[1,2-a]pyridin-2-yl]-1,2,4- | | | | | |
| | oxadiazol-3-yl}phenyl)oxy]propan-2-ol | | | | | |
| 224 | 2,5-dichloro-4-{5-[8-chloro-6- | A | nt | B | nt | nt |
| | (trifluoromethyl)imidazo[1,2-a]pyridin-2-yl]-1,3,4- | | | | | |
| | thiadiazol-2-yl}phenol | | | | | |
| 225 | 3-(4-{5-[8-bromo-6-(trifluoromethyl)imidazo[1,2- | nt | B¹ | nt | nt | nt |
| | a]pyridin-2-yl]-1,2,4-oxadiazol-3-yl}-2,5- | | | | | |
| | dichlorophenyl)-2-methylpropanoic acid | | | | | |
| 226 | 1-amino-3-[(2,5-dichloro-4-{5-[8-chloro-6- | A | A | nt | nt | C |
| | (trifluoromethyl)imidazo[1,2-a]pyridin-2-yl]-1,2,4- | | | | | |
| | oxadiazol-3-yl}phenyl)oxy]propan-2-ol | | | | | |
| 227 | 2-[(2,5-dichloro-4-{3-[8-chloro-6- | A | nt | A | nt | nt |
| | (trifluoromethyl)imidazo[1,2-a]pyridin-2-yl]-1,2,4- | | | | | |
| | oxadiazol-5-yl}phenyl)oxy]propan-1-ol | | | | | |
| 228 | 8-chloro-2-[5-(2,6-difluorophenyl)-1,2,4-oxadiazol-3-yl]- | B | nt | nt | nt | nt |
| | 6-(trifluoromethyl)imidazo[1,2-a]pyridine | | | | | |
| 229 | 8-chloro-2-[5-(2-chlorophenyl)-1,2,4-oxadiazol-3-yl]-6- | A | nt | nt | nt | nt |
| | (trifluoromethyl)imidazo[1,2-a]pyridine | | | | | |
| 230 | 8-chloro-2-[5-(2-fluorophenyl)-1,2,4-oxadiazol-3-yl]-6- | B | nt | nt | nt | nt |
| | (trifluoromethyl)imidazo[1,2-a]pyridine | | | | | |
| 231 | 1-[(4-{3-[8-chloro-6-(trifluoromethyl)imidazo[1,2- | E¹ | nt | nt | nt | nt |
| | a]pyridin-2-yl]-1,2,4-oxadiazol-5- | | | | | |
| | yl}phenyl)methyl]azetidine-3-carboxylic acid | | | | | |
| 232 | 8-chloro-2-[5-(2-chloro-4-fluorophenyl)-1,2,4-oxadiazol- | B | nt | nt | nt | nt |
| | 3-yl]-6-(trifluoromethyl)imidazo[1,2-a]pyridine | | | | | |
| 233 | 3-(5-chloro-4-{3-[8-chloro-6- | A | A | A | B | B |
| | (trifluoromethyl)imidazo[1,2-a]pyridin-2-yl]-1,2,4- | | | | | |
| | oxadiazol-5-yl}-2-fluorophenyl)propanoic acid | | | | | |
| 234 | N-(5-chloro-4-{3-[8-chloro-6- | A | nt | nt | nt | nt |
| | (trifluoromethyl)imidazo[1,2-a]pyridin-2-yl]-1,2,4- | | | | | |
| | oxadiazol-5-yl}-2-fluorophenyl)methanesulfonamide | | | | | |
| 235 | 3-(2-chloro-4-{3-[8-chloro-6- | A | B | nt | nt | nt |
| | (trifluoromethyl)imidazo[1,2-a]pyridin-2-yl]-1,2,4- | | | | | |
| | oxadiazol-5-yl}phenyl)propanoic acid | | | | | |
| 236 | N-(2-chloro-4-{3-[8-chloro-6- | E¹ | nt | nt | nt | nt |
| | (trifluoromethyl)imidazo[1,2-a]pyridin-2-yl]-1,2,4- | | | | | |
| | oxadiazol-5-yl}-6-fluorophenyl)methanesulfonamide | | | | | |
| 237 | 2-(4-{3-[8-chloro-6-(trifluoromethyl)imidazo[1,2- | E¹ | nt | nt | nt | nt |
| | a]pyridin-2-yl]-1,2,4-oxadiazol-5-yl}-2-fluoro-5- | | | | | |
| | methylphenyl)cyclopropanecarboxylic acid | | | | | |
| 238 | 3-(4-{3-[8-bromo-6-(trifluoromethyl)imidazo[1,2- | A | A | nt | nt | B |
| | a]pyridin-2-yl]-1,2,4-oxadiazol-5-yl}-5-chloro-2- | | | | | |
| | fluorophenyl)propanoic acid | | | | | |
| 239 | N-(3-chloro-4-{5-[8-chloro-6- | A | A | A | nt | B |
| | (trifluoromethyl)imidazo[1,2-a]pyridin-2-yl]-1,3,4- | | | | | |
| | thiadiazol-2-yl}phenyl)methanesulfonamide | | | | | |
| 240 | 3-{5-chloro-4-[3-(8-chloro-6-iodoimidazo[1,2-a]pyridin- | nt | A | nt | C | nt |
| | 2-yl)-1,2,4-oxadiazol-5-yl]-2-fluorophenyl}propanoic | | | | | |
| | acid | | | | | |
| 241 | 3-(2,5-dichloro-4-{3-[8-chloro-6- | nt | A | nt | nt | nt |
| | (trifluoromethyl)imidazo[1,2-a]pyridin-2-yl]-1,2,4- | | | | | |
| | oxadiazol-5-yl}phenyl)-2-methylpropanoic acid | | | | | |
| 242 | 3-(2,5-dichloro-4-{5-[8-chloro-6- | A | A | A | A | A |
| | (trifluoromethyl)imidazo[1,2-a]pyridin-2-yl]-1,2,4- | | | | | |
| | oxadiazol-3-yl}phenyl)propanoic acid | | | | | |
| 243 | 2-[(3-chloro-4-{5-[8-chloro-6- | A | B | nt | nt | nt |
| | (trifluoromethyl)imidazo[1,2-a]pyridin-2-yl]-1,3,4- | | | | | |
| | thiadiazol-2-yl}phenyl)oxy]ethanol | | | | | |
| 244 | 2-[(3-chloro-4-{5-[8-chloro-6- | A | B | nt | B | C |
| | (trifluoromethyl)imidazo[1,2-a]pyridin-2-yl]-1,3,4- | | | | | |
| | thiadiazol-2-yl}phenyl)oxy]ethanamine | | | | | |
| 245 | 3-[(3-chloro-4-{5-[8-chloro-6- | A | nt | B | nt | nt |
| | (trifluoromethyl)imidazo[1,2-a]pyridin-2-yl]-1,3,4- | | | | | |
| | thiadiazol-2-yl}phenyl)oxy]propane-1,2-diol | | | | | |
| 246 | 3-[(4-{5-[8-chloro-6-(trifluoromethyl)imidazo[1,2- | A | A | A | nt | nt |
| | a]pyridin-2-yl]-1,3,4-thiadiazol-2-yl}-2,6- | | | | | |
| | dimethylphenyl)oxy]propane-1,2-diol | | | | | |
| 247 | 3-[(5-chloro-4-{5-[8-chloro-6- | A | A | B | nt | nt |
| | (trifluoromethyl)imidazo[1,2-a]pyridin-2-yl]-1,3,4- | | | | | |
| | thiadiazol-2-yl}-2-fluorophenyl)oxy]propane-1,2-diol | | | | | |
| 248 | 2-[(5-chloro-4-{5-[8-chloro-6- | A | A | B | nt | nt |
| | (trifluoromethyl)imidazo[1,2-a]pyridin-2-yl]-1,3,4- | | | | | |
| | thiadiazol-2-yl}-2-fluorophenyl)oxy]propan-1-ol | | | | | |
| 249 | 1-amino-3-[(5-chloro-4-{5-[8-chloro-6- | A | nt | nt | nt | nt |
| | (trifluoromethyl)imidazo[1,2-a]pyridin-2-yl]-1,3,4- | | | | | |
| | thiadiazol-2-yl}-2-fluorophenyl)oxy]propan-2-ol | | | | | |
| 250 | 2-[(5-chloro-4-{5-[8-chloro-6- | A | nt | B | nt | nt |
| | (trifluoromethyl)imidazo[1,2-a]pyridin-2-yl]-1,3,4- | | | | | |
| | thiadiazol-2-yl}-2-fluorophenyl)oxy]propanoic acid | | | | | |
| 251 | 2-[(2,5-dichloro-4-{5-[8-chloro-6- | A | nt | A | nt | nt |
| | (trifluoromethyl)imidazo[1,2-a]pyridin-2-yl]-1,3,4- | | | | | |
| | thiadiazol-2-yl}phenyl)oxy]propan-1-ol | | | | | |
| 252 | (2S)-3-[(2,5-dichloro-4-{5-[8-chloro-6- | A | A | A | C | nt |
| | (trifluoromethyl)imidazo[1,2-a]pyridin-2-yl]-1,3,4- | | | | | |
| | thiadiazol-2-yl}phenyl)oxy]propane-1,2-diol | | | | | |
| 253 | [(3-chloro-4-{5-[8-chloro-6- | B | nt | nt | nt | nt |
| | (trifluoromethyl)imidazo[1,2-a]pyridin-2-yl]-1,2,4- | | | | | |
| | oxadiazol-3-yl}phenyl)oxy]acetic acid | | | | | |
| 254 | 2-[(3-chloro-4-{5-[8-chloro-6- | E¹ | nt | nt | nt | nt |
| | (trifluoromethyl)imidazo[1,2-a]pyridin-2-yl]-1,2,4- | | | | | |
| | oxadiazol-3-yl}phenyl)oxy]-2-methylpropanoic acid | | | | | |
| 255 | 3-[(3-chloro-4-{5-[8-chloro-6- | A | B | nt | nt | nt |
| | (trifluoromethyl)imidazo[1,2-a]pyridin-2-yl]-1,2,4- | | | | | |
| | oxadiazol-3-yl}phenyl)oxy]propanoic acid | | | | | |
| 256 | 2-[(3-chloro-4-{5-[8-chloro-6- | A | A | nt | C | B |
| | (trifluoromethyl)imidazo[1,2-a]pyridin-2-yl]-1,2,4- | | | | | |
| | oxadiazol-3-yl}phenyl)oxy]propan-1-ol | | | | | |
| 257 | 2-[(3-chloro-4-{5-[8-chloro-6- | A | A | B | nt | B |
| | (trifluoromethyl)imidazo[1,2-a]pyridin-2-yl]-1,2,4- | | | | | |
| | oxadiazol-3-yl}phenyl)oxy]ethanamine | | | | | |
| 258 | 2-[(3-chloro-4-{5-[8-chloro-6- | B | nt | nt | nt | nt |
| | (trifluoromethyl)imidazo[1,2-a]pyridin-2-yl]-1,2,4- | | | | | |
| | oxadiazol-3-yl}phenyl)oxy]propan-1-amine | | | | | |
| 259 | 2-[(5-chloro-4-{5-[8-chloro-6- | A | A | nt | A | B |
| | (trifluoromethyl)imidazo[1,2-a]pyridin-2-yl]-1,2,4- | | | | | |
| | oxadiazol-3-yl}-2-fluorophenyl)oxy]propan-1-ol | | | | | |
| 260 | 5-chloro-4-{5-[8-chloro-6-(trifluoromethyl)imidazo[1,2- | A | nt | nt | nt | nt |
| | a]pyridin-2-yl]-1,2,4-oxadiazol-3-yl}-2-fluorophenol | | | | | |
| 261 | 2-[(2-chloro-4-{5-[8-chloro-6- | A | nt | nt | nt | nt |
| | (trifluoromethyl)imidazo[1,2-a]pyridin-2-yl]-1,2,4- | | | | | |
| | oxadiazol-3-yl}-6-fluorophenyl)oxy]propan-1-ol | | | | | |
| 262 | 2-[(2-chloro-4-{5-[8-chloro-6- | D¹ | nt | nt | nt | nt |
| | (trifluoromethyl)imidazo[1,2-a]pyridin-2-yl]-1,2,4- | | | | | |
| | oxadiazol-3-yl}-6-fluorophenyl)oxy]-2-methylpropan-1- | | | | | |
| | ol | | | | | |
| 263 | 2-[(5-chloro-4-{5-[8-chloro-6- | A | A | A | A | B |
| | (trifluoromethyl)imidazo[1,2-a]pyridin-2-yl]-1,2,4- | | | | | |
| | oxadiazol-3-yl}-2-fluorophenyl)oxy]ethanol | | | | | |
| 264 | 8-chloro-2-{3-[2-chloro-5-fluoro-4-(pyrrolidin-3- | A | nt | nt | nt | nt |
| | yloxy)phenyl]-1,2,4-oxadiazol-5-yl}-6- | | | | | |
| | (trifluoromethyl)imidazo[1,2-a]pyridine | | | | | |
| 265 | N-(4-{5-[8-chloro-6-(trifluoromethyl)imidazo[1,2- | C¹ | nt | nt | nt | nt |
| | a]pyridin-2-yl]-1,2,4-oxadiazol-3-yl}-2,6- | | | | | |
| | difluorophenyl)methanesulfonamide | | | | | |
| 266 | 3-(4-{5-[8-bromo-6-(trifluoromethyl)imidazo[1,2- | A | B | nt | nt | nt |
| | a]pyridin-2-yl]-1,2,4-oxadiazol-3-yl}-3- | | | | | |
| | chlorophenyl)butanoic acid | | | | | |
| 267 | 3-(3-chloro-4-{5-[8-chloro-6- | A | B | nt | nt | nt |
| | (trifluoromethyl)imidazo[1,2-a]pyridin-2-yl]-1,2,4- | | | | | |
| | oxadiazol-3-yl}phenyl)-3-cyanopropanoic acid | | | | | |
| 268 | N-(2-chloro-4-{5-[8-chloro-6- | C | nt | nt | nt | nt |
| | (trifluoromethyl)imidazo[1,2-a]pyridin-2-yl]-1,2,4- | | | | | |
| | oxadiazol-3-yl}-6-fluorophenyl)methanesulfonamide | | | | | |
| 269 | N-{3-chloro-4-[5-(8-chloro-6-cyanoimidazo[1,2- | E¹ | nt | nt | nt | nt |
| | a]pyridin-2-yl)-1,2,4-oxadiazol-3- | | | | | |
| | yl]phenyl}methanesulfonamide | | | | | |
| 270 | N-{3-chloro-4-[5-(8-chloro-6-methylimidazo[1,2- | E¹ | nt | nt | nt | nt |
| | a]pyridin-2-yl)-1,2,4-oxadiazol-3- | | | | | |
| | yl]phenyl}methanesulfonamide | | | | | |
| 271 | 3-[(3-chloro-4-{5-[8-chloro-6- | B | nt | nt | nt | nt |
| | (trifluoromethyl)imidazo[1,2-a]pyridin-2-yl]-1,2,4- | | | | | |
| | oxadiazol-3-yl}phenyl)methyl]-1,2,4-oxadiazol-5(4H)- | | | | | |
| | one | | | | | |
| 272 | 1-(3-chloro-4-{5-[8-chloro-6- | A | B | nt | nt | nt |
| | (trifluoromethyl)imidazo[1,2-a]pyridin-2-yl]-1,2,4- | | | | | |
| | oxadiazol-3-yl}phenyl)propane-1,3-diol | | | | | |
| 273 | 8-chloro-2-[3-(2-chlorophenyl)-1,2,4-oxadiazol-5-yl]-6- | A | nt | nt | nt | nt |
| | (trifluoromethyl)imidazo[1,2-a]pyridine | | | | | |
| 274 | 8-chloro-2-[3-(2-chloro-4-fluorophenyl)-1,2,4-oxadiazol- | B | C | nt | nt | nt |
| | 5-yl]-6-(trifluoromethyl)imidazo[1,2-a]pyridine | | | | | |
| 275 | 8-chloro-2-(3-{2-chloro-4- | A | B | nt | nt | nt |
| | [(methylsulfonyl)methyl]phenyl}-1,2,4-oxadiazol-5-yl)- | | | | | |
| | 6-(trifluoromethyl)imidazo[1,2-a]pyridine | | | | | |
| 276 | (4S)-4-[(3-chloro-4-{5-[8-chloro-6- | A | nt | nt | nt | nt |
| | (trifluoromethyl)imidazo[1,2-a]pyridin-2-yl]-1,2,4- | | | | | |
| | oxadiazol-3-yl}phenyl)oxy]-L-proline | | | | | |
| 277 | 2-[(4-{5-[8-bromo-6-(trifluoromethyl)imidazo[1,2- | nt | A | A | A | nt |
| | a]pyridin-2-yl]-1,2,4-oxadiazol-3-yl}-2,5- | | | | | |
| | dichlorophenyl)oxy]ethanol | | | | | |
| 278 | 2-{[(2,5-dichloro-4-{5-[8-chloro-6- | nt | A | A | A | nt |
| | (trifluoromethyl)imidazo[1,2-a]pyridin-2-yl]-1,2,4- | | | | | |
| | oxadiazol-3-yl}phenyl)oxy]methyl}propane-1,3-diol | | | | | |
| 279 | 2-[(2,6-dichloro-4-{5-[8-chloro-6- | nt | B | nt | nt | nt |
| | (trifluoromethyl)imidazo[1,2-a]pyridin-2-yl]-1,2,4- | | | | | |
| | oxadiazol-3-yl}phenyl)oxy]ethyl acetate | | | | | |
| 280 | 2-[(2,6-dichloro-4-{5-[8-chloro-6- | nt | A | nt | nt | nt |
| | (trifluoromethyl)imidazo[1,2-a]pyridin-2-yl]-1,2,4- | | | | | |
| | oxadiazol-3-yl}phenyl)oxy]ethanol | | | | | |
| 281 | 8-bromo-2-[3-(2-chloro-4-fluorophenyl)-1,2,4-oxadiazol- | B | nt | nt | nt | nt |
| | 5-yl]-6-(trifluoromethyl)imidazo[1,2-a]pyridine | | | | | |
| 282 | 3-(5-chloro-4-{5-[8-chloro-6- | A | A | nt | nt | nt |
| | (trifluoromethyl)imidazo[1,2-a]pyridin-2-yl]-1,3,4- | | | | | |
| | oxadiazol-2-yl}-2-fluorophenyl)propanoic acid | | | | | |
| 283 | 3-(3-chloro-4-{5-[8-chloro-6- | A | A | nt | B | B |
| | (trifluoromethyl)imidazo[1,2-a]pyridin-2-yl]-1,3,4- | | | | | |
| | thiadiazol-2-yl}phenyl)propanoic acid | | | | | |
| 285 | 3-(2,5-dichloro-4-{5-[8-chloro-6- | nt | A | nt | D | nt |
| | (trifluoromethyl)imidazo[1,2-a]pyridin-2-yl]-1,3,4- | | | | | |
| | thiadiazol-2-yl}phenyl)-2-methylpropanoic acid | | | | | |
| 286 | 3-(4-{5-[8-bromo-6-(trifluoromethyl)imidazo[1,2- | nt | A | A | D | nt |
| | a]pyridin-2-yl]-1,3,4-thiadiazol-2-yl}-2,5- | | | | | |
| | dichlorophenyl)propanoic acid | | | | | |
| 287 | 1-[(4-{5-[8-bromo-6-(trifluoromethyl)imidazo[1,2- | nt | A | A | B | nt |
| | a]pyridin-2-yl]-1,2,4-oxadiazol-3-yl}-2,5- | | | | | |
| | dichlorophenyl)oxy]-2-methylpropan-2-ol | | | | | |
| 288 | 2-[(2,5-dichloro-4-{5-[8-chloro-6- | A | A | A | A | nt |
| | (trifluoromethyl)imidazo[1,2-a]pyridin-2-yl]-1,2,4- | | | | | |
| | oxadiazol-3-yl}phenyl)oxy]ethanol | | | | | |
| 289 | (2R)-3-[(2,5-dichloro-4-{5-[8-chloro-6- | A | A | A | A | nt |
| | (trifluoromethyl)imidazo[1,2-a]pyridin-2-yl]-1,2,4- | | | | | |
| | oxadiazol-3-yl}phenyl)oxy]propane-1,2-diol | | | | | |
| 290 | 8-chloro-6-(trifluoromethyl)-2-{3-[4- | D | nt | nt | nt | nt |
| | (trifluoromethyl)pyridin-3-yl]-1,2,4-oxadiazol-5- | | | | | |
| | yl}imidazo[1,2-a]pyridine | | | | | |
| 291 | 2-[3-(2-chloro-4-fluorophenyl)-1,2,4-oxadiazol-5-yl]-6- | B | nt | nt | nt | nt |
| | (trifluoromethyl)imidazo[1,2-a]pyridine-8-carbonitrile | | | | | |
| 292 | 1-[(2,5-dichloro-4-{5-[8-chloro-6- | A | A | A | B | nt |
| | (trifluoromethyl)imidazo[1,2-a]pyridin-2-yl]-1,2,4- | | | | | |
| | oxadiazol-3-yl}phenyl)oxy]propan-2-ol | | | | | |
| 293 | (4-{5-[8-chloro-6-(trifluoromethyl)imidazo[1,2- | C | nt | nt | nt | nt |
| | a]pyridin-2-yl]-1,2,4-oxadiazol-3-yl}-1H-indol-1- | | | | | |
| | yl)acetic acid | | | | | |
| 294 | 3-[(4-{5-[8-bromo-6-(trifluoromethyl)imidazo[1,2- | nt | nt | A | nt | nt |
| | a]pyridin-2-yl]-1,2,4-oxadiazol-3-yl}-2,5- | | | | | |
| | dichlorophenyl)oxy]-1,1,1-trifluoropropan-2-ol | | | | | |
| 295 | (2R)-1-[(2,5-dichloro-4-{5-[8-chloro-6- | nt | A | A | A | nt |
| | (trifluoromethyl)imidazo[1,2-a]pyridin-2-yl]-1,2,4- | | | | | |
| | oxadiazol-3-yl}phenyl)oxy]propan-2-ol | | | | | |
| 296 | 3-(2-chloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}-6-fluorophenyl)propanoic acid | | | | | |
| 297 | 3-{2-chloro-4-[3-(8-chloro-6-iodoimidazo[1,2-a]pyridin- | nt | nt | A | nt | nt |
| | 2-yl)-1,2,4-oxadiazol-5-yl]-5-methylphenyl}propanoic | | | | | |
| | acid | | | | | |
| 298 | 1-[(5-chloro-4-{5-[8-chloro-6- | nt | nt | B¹ | nt | nt |
| | (trifluoromethyl)imidazo[1,2-a]pyridin-2-yl]-1,3,4- | | | | | |
| | thiadiazol-2-yl}-2-fluorophenyl)oxy]propan-2-amine | | | | | |
| 299 | 2-(2,5-dichloro-4-{3-[8-chloro-6- | nt | nt | B | nt | nt |
| | (trifluoromethyl)imidazo[1,2-a]pyridin-2-yl]-1,2,4- | | | | | |
| | oxadiazol-5-yl}phenyl)cyclopropanecarboxylic acid | | | | | |
| 300 | 3-{5-chloro-2-fluoro-4-[5-(6-iodo-5-methylimidazo[1,2- | A | A | nt | D | B |
| | a]pyridin-2-yl)-1,2,4-oxadiazol-3-yl]phenyl}propanoic | | | | | |
| | acid | | | | | |
| 301 | 3-[5-chloro-4-(5-{8-chloro-6-[(1- | A | A | A | B | A |
| | methylethyl)oxy]imidazo[1,2-a]pyridin-2-yl}-1,2,4- | | | | | |
| | oxadiazol-3-yl)-2-fluorophenyl]propanoic acid | | | | | |
| 302 | 3-{5-chloro-4-[5-(8-chloro-6-iodoimidazo[1,2-a]pyridin- | A | A | A | B | A |
| | 2-yl)-1,2,4-oxadiazol-3-yl]-2-fluorophenyl}propanoic | | | | | |
| | acid | | | | | |
| 303 | 2-amino-3-[(2,5-dichloro-4-{5-[8-chloro-6- | nt | A | A | nt | nt |
| | (trifluoromethyl)imidazo[1,2-a]pyridin-2-yl]-1,2,4- | | | | | |
| | oxadiazol-3-yl}phenyl)oxy]propan-1-ol | | | | | |
| 304 | 8-chloro-2-{3-[2,5-dichloro-4-({[(4R)-2,2-dimethyl-1,3- | nt | nt | A | nt | nt |
| | dioxolan-4-yl]methyl}oxy)phenyl]-1,2,4-oxadiazol-5- | | | | | |
| | yl}-6-(trifluoromethyl)imidazo[1,2-a]pyridine | | | | | |
| 305 | 1-[(4-{5-[8-bromo-6-(trifluoromethyl)imidazo[1,2- | nt | nt | A | nt | nt |
| | a]pyridin-2-yl]-1,2,4-oxadiazol-3-yl}-2,5- | | | | | |
| | dichlorophenyl)oxy]propan-2-one | | | | | |
| 306 | 2-(4-{5-[8-bromo-6-(trifluoromethyl)imidazo[1,2- | nt | nt | A | nt | nt |
| | a]pyridin-2-yl]-1,2,4-oxadiazol-3-yl}-2,5- | | | | | |
| | dichlorophenyl)cyclopropanecarboxylic acid | | | | | |
| 307 | 2-[(2,5-dichloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}phenyl)oxy]propane-1,3-diol | | | | | |
| 308 | 2-[(5-chloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}-2-fluorophenyl)amino]ethanol | | | | | |
| 309 | (2S)-2-amino-3-[(5-chloro-2-fluoro-4-{5-[6- | nt | nt | A | nt | nt |
| | (trifluoromethyl)imidazo[1,2-a]pyridin-2-yl]-1,3,4- | | | | | |
| | thiadiazol-2-yl}phenyl)oxy]propan-1-ol | | | | | |
| 310 | (2S)-2-amino-3-[(5-chloro-2-fluoro-4-{5-[6- | nt | nt | A | nt | nt |
| | (methyloxy)imidazo[1,2-a]pyridin-2-yl]-1,3,4-thiadiazol- | | | | | |
| | 2-yl}phenyl)oxy]propan-1-ol | | | | | |
| 311 | 3-[(2,5-dichloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}phenyl)oxy]-2,2-difluoropropan-1-ol | | | | | |
| 312 | (2R)-1-[(4-{5-[8-bromo-6-(trifluoromethyl)imidazo[1,2- | nt | A | A | nt | nt |
| | a]pyridin-2-yl]-1,2,4-oxadiazol-3-yl}-2,5- | | | | | |
| | dichlorophenyl)oxy]propan-2-ol | | | | | |
| 313 | (2S)-1-[(4-{5-[8-bromo-6-(trifluoromethyl)imidazo[1,2- | nt | A | A | nt | nt |
| | a]pyridin-2-yl]-1,2,4-oxadiazol-3-yl}-2,5- | | | | | |
| | dichlorophenyl)oxy]propan-2-ol | | | | | |
| 314 | 3-(2,6-dichloro-4-{3-[8-chloro-6- | A | A | nt | nt | nt |
| | (trifluoromethyl)imidazo[1,2-a]pyridin-2-yl]-1,2,4- | | | | | |
| | oxadiazol-5-yl}phenyl)propanoic acid | | | | | |
| 315 | 3-(2,5-dichloro-4-{3-[8-chloro-6- | A | A | A | B | nt |
| | (trifluoromethyl)imidazo[1,2-a]pyridin-2-yl]-1,2,4- | | | | | |
| | oxadiazol-5-yl}phenyl)propanoic acid | | | | | |
| 316 | 1-[(4-{5-[8-bromo-6-(trifluoromethyl)imidazo[1,2- | nt | nt | A | nt | nt |
| | a]pyridin-2-yl]-1,2,4-oxadiazol-3-yl}-2,5- | | | | | |
| | dichlorophenyl)oxy]propan-2-ol | | | | | |
| 317 | (2R)-3-[(4-{5-[8-bromo-6-(trifluoromethyl)imidazo[1,2- | nt | A | A | nt | nt |
| | a]pyridin-2-yl]-1,2,4-oxadiazol-3-yl}-2,5- | | | | | |
| | dichlorophenyl)oxy]propane-1,2-diol | | | | | |
| 318 | 1-[(2,5-dichloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}phenyl)oxy]-3-(methyloxy)propan-2-ol | | | | | |
| 319 | methyl 3-[(2,5-dichloro-4-{5-[8-chloro-6- | nt | nt | B¹ | nt | nt |
| | (trifluoromethyl)imidazo[1,2-a]pyridin-2-yl]-1,2,4- | | | | | |
| | oxadiazol-3-yl}phenyl)oxy]-2-hydroxy-2- | | | | | |
| | methylpropanoate | | | | | |
| 320 | 2-(2,5-dichloro-4-{5-[8-chloro-6- | nt | nt | A | nt | nt |
| | (trifluoromethyl)imidazo[1,2-a]pyridin-2-yl]-1,3,4- | | | | | |
| | thiadiazol-2-yl}phenyl)cyclopropanecarboxylic acid | | | | | |
| 321 | 2-amino-3-[(5-chloro-4-{5-[8-chloro-6- | nt | A | A | nt | nt |
| | (trifluoromethyl)imidazo[1,2-a]pyridin-2-yl]-1,3,4- | | | | | |
| | thiadiazol-2-yl}-2-fluorophenyl)oxy]propan-1-ol | | | | | |
| 322 | 1:1 mixture of (2R)-3-[(2,5-dichloro-4-{5-[8-chloro-6- | nt | A | A | nt | nt |
| | (trifluoromethyl)imidazo[1,2-a]pyridin-2-yl]-1,2,4- | | | | | |
| | oxadiazol-3-yl}phenyl)oxy]-2-hydroxypropyl dihydrogen | | | | | |
| | phosphate and (S)-1-(2,5-dichloro-4-(5-(8-chloro-6- | | | | | |
| | (trifluoromethyl)imidazo[1,2-a]pyridin-2-yl)-1,2,4- | | | | | |
| | oxadiazol-3-yl)phenoxy)-3-hydroxypropan-2-yl | | | | | |
| | dihydrogen phosphate | | | | | |
| 323 | 1-[(2,5-dichloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}phenyl)amino]propan-2-ol | | | | | |
| 324 | (1R)-2-[(4-{5-[8-bromo-6-(trifluoromethyl)imidazo[1,2- | nt | A | A | nt | nt |
| | a]pyridin-2-yl]-1,2,4-oxadiazol-3-yl}-2,5- | | | | | |
| | dichlorophenyl)oxy]-1-methylethyl dihydrogen | | | | | |
| | phosphate | | | | | |
| 325 | (1S)-2-[(4-{5-[8-bromo-6-(trifluoromethyl)imidazo[1,2- | nt | nt | A | nt | nt |
| | a]pyridin-2-yl]-1,2,4-oxadiazol-3-yl}-2,5- | | | | | |
| | dichlorophenyl)oxy]-1-methylethyl dihydrogen | | | | | |
| | phosphate | | | | | |
| 326 | (1S)-2-[(2,5-dichloro-4-{5-[8-chloro-6- | nt | A | A | nt | nt |
| | (trifluoromethyl)imidazo[1,2-a]pyridin-2-yl]-1,2,4- | | | | | |
| | oxadiazol-3-yl}phenyl)oxy]-1-methylethyl hydrogen | | | | | |
| | sulfate | | | | | |
| 327 | 3-[(2,5-dichloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}phenyl)oxy]-2-hydroxy-2- | | | | | |
| | methylpropanoic acid | | | | | |
| 328 | (1R)-2-[(2,5-dichloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}phenyl)oxy]-1-methylethyl dihydrogen | | | | | |
| | phosphate | | | | | |
| 329 | 3-(2-chloro-4-{3-[8-chloro-6- | nt | nt | A | nt | nt |
| | (trifluoromethyl)imidazo[1,2-a]pyridin-2-yl]-1,2,4- | | | | | |
| | oxadiazol-5-yl}-5-methylphenyl)propanoic acid | | | | | |
| 330 | 3-[(2,5-dichloro-4-{5-[8-chloro-6- | nt | A | A | nt | nt |
| | (trifluoromethyl)imidazo[1,2-a]pyridin-2-yl]-1,2,4- | | | | | |
| | oxadiazol-3-yl}phenyl)oxy]-2-methylpropane-1,2-diol | | | | | |
| 331 | (1R)-2-[(2,5-dichloro-4-{5-[8-chloro-6- | nt | nt | A | nt | nt |
| | (trifluoromethyl)imidazo[1,2-a]pyridin-2-yl]-1,2,4- | | | | | |
| | oxadiazol-3-yl}phenyl)oxy]-1-methylethyl hydrogen | | | | | |
| | sulfate | | | | | |
| 332 | N-[(3-chloro-4-{5-[8-chloro-6- | nt | nt | A | nt | nt |
| | (trifluoromethyl)imidazo[1,2-a]pyridin-2-yl]-1,3,4- | | | | | |
| | thiadiazol-2-yl}phenyl)methyl]methanesulfonamide | | | | | |
| 333 | 8-chloro-2-[3-(2,5-dichloro-4-{[2- | nt | nt | B¹ | nt | nt |
| | (methylsulfonyl)ethyl]oxy}phenyl)-1,2,4-oxadiazol-5- | | | | | |
| | yl]-6-(trifluoromethyl)imidazo[1,2-a]pyridine | | | | | |
| 334 | (2R)-2-Amino-3-[(5-chloro-4-{5-[8-chloro-6- | nt | A | A | nt | nt |
| | (trifluoromethyl)imidazo[1,2-a]pyridin-2-yl]-1,3,4- | | | | | |
| | thiadiazol-2-yl}-2-fluorophenyl)oxy]propan-1-ol | | | | | |
| 335 | (2S)-2-Amino-3-[(5-chloro-4-{5-[8-chloro-6- | nt | nt | A | nt | nt |
| | (trifluoromethyl)imidazo[1,2-a]pyridin-2-yl]-1,3,4- | | | | | |
| | thiadiazol-2-yl}-2-fluorophenyl)oxy]propan-1-ol | | | | | |
| 336 | 1-amino-3-[(2,5-dichloro-4-{5-[8-chloro-6- | nt | nt | A | nt | nt |
| | (trifluoromethyl)imidazo[1,2-a]pyridin-2-yl]-1,3,4- | | | | | |
| | thiadiazol-2-yl}phenyl)oxy]propan-2-ol | | | | | |
| 337 | 2,5-dichloro-4-{5-[8-chloro-6- | nt | nt | B | nt | nt |
| | (trifluoromethyl)imidazo[1,2-a]pyridin-2-yl]-1,2,4- | | | | | |
| | oxadiazol-3-yl}aniline | | | | | |
| 338 | 5-chloro-4-{5-[8-chloro-6-(trifluoromethyl)imidazo[1,2- | nt | nt | B | nt | nt |
| | a]pyridin-2-yl]-1,3,4-thiadiazol-2-yl}-2-fluorophenol | | | | | |
| 339 | 2-amino-3-({4-[5-(6-bromoimidazo[1,2-a]pyridin-2-yl)- | nt | nt | A | nt | nt |
| | 1,3,4-thiadiazol-2-yl]-5-chloro-2- | | | | | |
| | fluorophenyl}oxy)propan-1-ol | | | | | |
| 340 | 1-[(2,5-dichloro-4-{5-[6-(trifluoromethyl)imidazo[1,2- | nt | nt | E¹ | nt | nt |
| | a]pyridin-2-yl]-1,3,4-thiadiazol-2-yl}phenyl)oxy]propan- | | | | | |
| | 2-amine | | | | | |
| 341 | 5-(8-chloro-6-(trifluoromethyl)imidazol(1,2-a)pyridine- | nt | nt | C¹ | nt | Nt |
| | 2-yl-3-(2,5-dichloro-4-methoxyphenyl)-1,2,4-oxadizole | | | | | |
| 420 | (2R)-2-amino-3-[(2,5-dichloro-4-{5-[8-chloro-6- | nt | nt | B | nt | nt |
| | (trifluoromethyl)imidazo[1,2-a]pyridin-2-yl]-1,3,4- | | | | | |
| | thiadiazol-2-yl}phenyl)oxy]propan-1-ol | | | | | |
| 421 | 2-amino-3-[(5-chloro-2-fluoro-4-{5-[7- | nt | nt | B | nt | nt |
| | (trifluoromethyl)imidazo[1,2-a]pyridin-2-yl]-1,3,4- | | | | | |
| | thiadiazol-2-yl}phenyl)oxy]propan-1-ol | | | | | |

¹Relative EC₅₀ was measured.

### Biological Examples 4-6

### In vivo models

### Example 4

### Delayed-Type Hypersensitivity (DTH) Model

Blood lymphocyte numbers, essential for the development of efficient immune responses, are maintained by recirculation through secondary lymphoid organs. Signaling of S1P through S1P1 has been shown to exclusively modulate egress of lymphocyte including 70% of activated T cells from lymph nodes. Delayed-type hypersensitivity (DTH) is an immune response mediated by a variety of inflammatory cells, including neutrophils, macrophages and T cells (Kobayashi et al. 2001, Black 1999). DTH develops in two phases, a sensitization phase, in which T cells are sensitized and memory T cells are formed, and an elicitation phase, in which T cell recall responses are induced upon secondary challenge with antigen. This second phase results in recruitment of inflammatory cells such as neutrophils and macrophages to the injection site of an intradermally applied antigen in a previously sensitized host, which causes swelling 24 h to 48 h post antigen challenge. The DTH assay (primarily done in mice) is an in vivo manifestation of a cell-mediated immunity reaction, and the response to antigen representation modulated by immunosuppressive treatment can be measured.

C57B1/6 male mice (10 mice per group) were immunized on day zero by subcutaneous injection at the base of the tail with 100 μL of 2 mg/mL methylated BSA emulsified with Complete Freunds Adjuvans (CFA, Sigma). Once-daily for twice-daily administration of a Compound of the Invention occurred for 10 days. On day 10 after immunization, mice received a second booster injection at the base of tail of an emulsified mixture of 2 mg/mL methylated BSA in Incomplete Freund's Adjuvans. On day 13 animals were challenged subcutaneously in the left hind footpad with 20 μL of 10 mg/mL methylated BSA in sterile water (water for injection). Animals were injected with an equal volume of sterile water into the right hind footpad as a control. Twenty four hours later (dose day 14) the right and left hind foot paws were transected at the medial and lateral malleolus, weighed, and the weight difference induced by injected antigen determined and compared to weight differences of vehicle treated non-sensitized and sensitized control groups. The increase in paw weights comparing left and right hind paw for each treatment group were analyzed for differences of treatment with a Compound of the Invention compared to vehicle control group using the Mann-Whitney non-parametric test statistic with minimal significance level set at p<0.05.

### Example 5

### Allograft Model

The rodent allograft model is an in vivo assay for assessing tissue rejection (ie, from transplantation) in response to chronic and/or sub-chronic immunosuppressive treatment (Chiba et al, 2005). Rejection is caused by T lymphocytes of the recipient responding to the foreign major histocompatibility complex of the donor graft. The transplanted organ (eg, skin) represents a continuous source of HLA alloantigens capable of inducing a rejection response at any time post transplantation. Because it cannot be eliminated, the allograft continuously activates the immune system, resulting in lifelong overproduction of cytokines, constant cytotoxic activity, and sustained alteration in the graft vasculature. Therefore, lifelong immunosuppression is required to ensure allograft survival. In this model skin from donor rats (male Lewis; histocompatibility RT-1¹) is surgically engrafted onto a dorsal region of recipient rats (male F344; histocompatibility RT-1^{1v1}). Administration of compound occurs immediately after surgery for a predetermined duration. Skin allografts are monitored daily for rejection.

On the day of surgery male Lewis donor rats were anesthetized with Isoflurane and skin aseptically harvested from the tail. Male F344 acceptor rats (8 per group) previously shaved (1-2 days prior to surgery) in the designated engraftment area were anesthetized with Isoflurane and a full thickness skin graft bed on the medial flank removed and discarded. The skin graft bed removed was equivalent in size to the donor skin to be engrafted. The prepared donor skin was then secured on the prepared graft bed with spot tissue glue or by 4 to 8 nonsilk sutures, and covered with sterile Vaseline gauze and wrapped with a bandage. All surgery took place on heated pads with sterile surgical equipment. Animals were monitored and turned every 20 minutes until ambulatory before returning to cages, water and food. Initiation of administration of a Compound of the Invention (orally once-daily or twice-daily) occurred when the animals fully recovered from anesthesia for a period of 14 days. On day 5 post-surgery, surgical bandaging was removed and the grafts assessed daily for rejection (necrosis of the graft tissue following by scabbing and sloughing from the graft bed site). An allograft was scored as “rejected” when it sloughed from the graft bed site. A positive effect in this model was delayed rejection of the allograft in response to treatment with a Compound of the Invention when compared to vehicle-treated control animals.

### Example 6

### Experimental Autoimmune Encephalomyelitis (EAE) Model

Multiple sclerosis is a demyelinating disease of the CNS. The main features of the disease are focal areas of demyelination and inflammation mediated by macrophages and t-lymphocytes. These cells develop in the peripheral lymphoid organs and travel to the CNS causing an autoimmune response. The development of T cells is controlled largely by the expression of various cytokines as well as cellular adhesion molecules. The EAE model today is the most thoroughly studied animal model for human autoimmune diseases. Mice are immunized with myelin-derived peptide PLP and clinical parameters of disease (bodyweight loss and paralysis) are monitored daily. The endpoint is the analysis of the extent of inflammation in brain and spinal cord.

C57B1/6 mice develop chronic paralysis after immunization with MOG₃₅₋₅₅ peptide. Mice develop EAE 8-14 days after immunization and stay chronically paralyzed for 30-40 days after onset of disease. Female C57B1/6 mice are subcutaneously injected with MOG₃₅₋₅₅ peptide emulsified in Complete Freund's Adjuvant at two sites on the back, injecting 0.1 mL at each site. Within 2 h of injection, pertussis toxin (aids in brain penetration of the MOG peptide) is administered intraperitoneally. A second injection of pertussis toxin is administered 22-26 h after the MOG₃₅₋₅₅ peptide injection. Onset of EAE is typically 7 days after immunization. EAE is scored on a scale of 0-5 with 0 being no obvious changes in motor functions, while 5 indicates complete paralysis. Mice are orally administered a Compound of the Invention (once-daily or twice-daily) on the day of MOG₃₅₋₅₅ peptide injection and monitored for paralysis and compared to vehicle-treated control animals. A positive effect in this model is delayed onset/severity of EAE.

The foregoing invention has been described in some detail by way of illustration and example, for purposes of clarity and understanding. The invention has been described with reference to various specific embodiments and techniques. However, it should be understood that many variations and modifications may be made while remaining within the spirit and scope of the invention. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled. All patents, patent applications and publications cited in this application are hereby incorporated by reference in their entirety for all purposes to the same extent as if each individual patent, patent application or publication were so individually denoted.

1. A compound of Formula I: 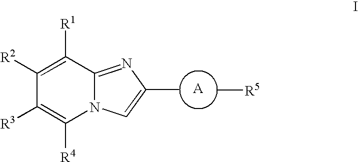
or a single stereoisomer or a mixture of isomers thereof, all optionally as a pharmaceutically acceptable salt thereof, where
R¹ is hydrogen, halo, cyano, C₁₋₆-alkoxy, amino, C₁₋₆-alkylamino, or di-(C₁₋₆-alkyl)amino;
R² is hydrogen, methyl, or methoxy;
R³ is hydrogen, C₁₋₆-alkyl, C₁₋₆-alkylsulfonyl, halo, halo-C₁₋₆-alkyl, C₁₋₆-alkoxy, optionally substituted phenoxy, cyano, C₁₋₆-alkylsulfonylamino, or nitro;
R⁴ is hydrogen or C₁₋₆-alkyl; 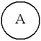
is a 5-membered heteroarylene;
R⁵ is phenyl substituted with R⁶, R⁷, and R⁸; or
R⁵ is heteroaryl optionally substituted with one or two R¹⁵ groups independently selected from C₁₋₆-alkyl; carboxy; halo-C₁₋₆-alkyl; carboxy-C₁₋₆-alkyl; C₁₋₆-alkoxycarbonyl-C₁₋₆-alkyl; and C₁₋₆-alkyl substituted with one —C(O)NR¹⁴R^{14a} group where R¹⁴ is hydrogen, C₁₋₆-alkyl, halo-C₁₋₆-alkyl, or hydroxy-C₁₋₆-alkyl and R^{14a} is hydrogen, C₁₋₆-alkyl, halo-C₁₋₆-alkyl, hydroxy-C₁₋₆-alkyl, or C₁₋₆-alkyl substituted with —O—Si(C₁₋₆-alkyl)₃; provided that when the R⁵ heteroaryl is pyridinyl or thienyl, then the pyridinyl and thienyl is substituted with one R¹⁵ and optionally substituted with an independently-selected second R¹⁵;
R⁶ is halo; hydroxy; cyano; —C(O)H; carboxy; alkoxycarbonyl; —C(═NOH)NH₂; —C(O)R¹⁷; —OR¹³; —NR¹¹R^{11a}; —NR¹²S(O)₂R^{12a}; optionally substituted heteroaryl; optionally substituted heterocycloalkyl; C₁₋₆-alkyl optionally substituted with 1, 2, 3, 4, or 5 R⁹ groups; C₂₋₆-alkenyl optionally substituted with one or two groups independently selected from carboxy and alkoxycarbonyl; or cycloalkyl optionally substituted with 1 or 2 groups independently selected from hydroxy-C₁₋₆-alkyl, alkoxycarbonyl, carboxy, and —C(O)NR¹⁰R^{10a};
R⁷ and R⁸ are independently hydrogen, halo, halo-C₁₋₆-alkyl, or C₁₋₆-alkyl;
each R⁹, when R⁹ is present, is independently cyano; hydroxy; halo; —C(O)H; —C(O)NR¹⁰R^{10a}; —C(O)OR¹⁰; —NR¹¹R^{11a}; —NR¹²S(O)₂R^{12a}; —P(O)(OR¹⁶)₂; —OP(O)(OR¹⁶)₂; —OS(O)₂OH; —S(O)ₙR¹⁸; —C(═NOH)NH₂; optionally substituted heteroaryl; or heterocycloalkyl optionally substituted with 1, 2, or 3 groups independently selected from hydroxy, carboxy, alkoxycarbonyl, C₁₋₆-alkyl, hydroxy-C₁₋₆-alkyl, and alkoxycarbonylamino;
R¹⁰ is hydrogen, C₁₋₆-alkyl, C₂₋₆-alkenyl, or C₂₋₆-alkynyl;
R^{10a} is hydrogen, C₁₋₆-alkyl, C₂₋₆-alkenyl, or C₂₋₆-alkynyl;
R^{10b} is hydrogen, C₁₋₆-alkyl, hydroxy-C₁₋₆-alkyl, carboxy-C₁₋₆-alkyl, halo-C₁₋₆-alkyl, C₂₋₆-alkenyl, C₂₋₆-alkynyl, or C₁₋₆-alkyl substituted with one or two groups independently selected from —P(O)(OR¹⁶)₂, —OP(O)(OR¹⁶)₂, —OS(O)₂OH, and —OSi(C₁₋₆-alkyl)₃;
R¹¹ is hydrogen, C₁₋₆-alkyl, C₂₋₆-alkenyl, or C₂₋₆-alkynyl;
R^{11a} is hydrogen, C₁₋₆-alkyl, C₂₋₆-alkenyl, C₂₋₆-alkynyl, C₁₋₆-alkylsulfonyl, C₁₋₆-alkoxycarbonyl, carboxy-C₁₋₆-alkyl, or hydroxy-C₁₋₆-alkyl;
R¹² is hydrogen, C₁₋₆-alkyl, C₂₋₆-alkenyl, or C₂₋₆-alkynyl;
R^{12a} is C₁₋₆-alkyl, C₂₋₆-alkenyl, C₂₋₆-alkynyl, amino-C₁₋₆-alkyl, C₁₋₆-alkylamino-C₁₋₆-alkyl, or di-(C₁₋₆-alkyl)amino-C₁₋₆-alkyl;
R¹³ is C₂₋₆-alkenyl; C₁₋₆-alkyl optionally substituted with 1, 2, 3, or 4 groups independently selected from halo, hydroxy, alkoxy, C₁₋₆-alkylsulfanyl, C₁₋₆-alkylsulfonyl, cyano, —C(O)OR¹⁰, —OC(O)R¹⁰, —C(O)R^{10b}, —NR¹¹R^{11a},—P(O)(OR¹⁶)₂, —OP(O)(OR¹⁶)₂, —OS(O)₂OH, —OSi(C₁₋₆-alkyl)₃, and heterocycloalkyl where the heterocycloalkyl is optionally substituted with one, two, or three groups independently selected from C₁₋₆-alkyl, carboxy, C₁₋₆-alkoxycarbonyl, C₁₋₆-alkoxycarbonylamino, and phenyl; or heterocycloalkyl optionally substituted with 1 or 2 groups independently selected from C₁₋₆-alkyl, carboxy, hydroxy-C₁₋₆-alkyl, carboxy-C₁₋₆-alkyl, and phenyl;
each R¹⁶ is independently hydrogen or C₁₋₆-alkyl;
R¹⁷ is amino, halo or C₁₋₆-alkyl substituted with one or two groups independently selected from carboxy or C₁₋₆-alkoxycarbonyl;
R¹⁸ is C₁₋₆-alkyl; and
n is 0, 1, or 2;
provided that when R⁵ is phenyl substituted with R⁶, R⁷, and R⁸ and
a) 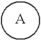
is furanyl and R⁶ is halo or cyano
b) 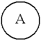
is thienyl and R⁶ is unsubstituted C₁₋₆-alkyl,
c) 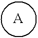
is oxadiazolyl, R⁶ is —OR¹³, and R¹³ is unsubstituted C₁₋₆-alkyl, or
d) 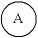
is oxazoyl, R⁶ is C₁₋₆-alkyl substituted with 3R⁹, and each R⁹ is halo, then at least one of R⁷ and R⁸ is not hydrogen.

2. The Compound of claim 1 where 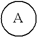
is oxadiazolyl or thiadiazolyl; optionally as a single stereoisomer or a mixture of isomers thereof and all additionally optionally as a pharmaceutically acceptable salt thereof.

3. The Compound of claim 1 where 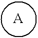
is oxadiazolyl; optionally as a single stereoisomer or a mixture of isomers thereof and all additionally optionally as a pharmaceutically acceptable salt thereof.

4. The Compound of claim 1 where 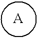
is thiadiazolyl; optionally as a single stereoisomer or a mixture of isomers thereof and all additionally optionally as a pharmaceutically acceptable salt thereof.

5. The Compound of claim 2 where R⁵ is phenyl substituted with R⁶, R⁷, and R⁸; optionally as a single stereoisomer or a mixture of isomers thereof and all additionally optionally as a pharmaceutically acceptable salt thereof.

6. The Compound of claim 5 where R¹ is halo, R² and R⁴ are hydrogen, and R³ is halo or haloalkyl; optionally as a single stereoisomer or a mixture of isomers thereof and all additionally optionally as a pharmaceutically acceptable salt thereof.

7. The Compound of claim 6 where R⁵ is according to formula (b): 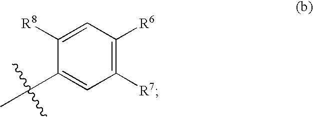
optionally as a single stereoisomer or a mixture of isomers thereof and all additionally optionally as a pharmaceutically acceptable salt thereof.

8. The Compound of claim 7 where R⁸ is halo and R⁷ is hydrogen or halo; optionally as a single stereoisomer or a mixture of isomers thereof and all additionally optionally as a pharmaceutically acceptable salt thereof.

9. The Compound of claim 8 where R⁶ is alkyl substituted with 1, 2, 3, 4, or 5 R⁹ groups; optionally as a single stereoisomer or a mixture of isomers thereof and all additionally optionally as a pharmaceutically acceptable salt thereof.

10. The Compound of claim 8 where R⁶ is alkyl substituted with one R⁹ selected from alkylsulfonyl, hydroxy, —C(O)NR¹⁰R^{10a}, and —C(O)OR¹⁰ and optionally additionally substituted with a second R⁹ selected from hydroxy and cyano; optionally as a single stereoisomer or a mixture of isomers thereof and all additionally optionally as a pharmaceutically acceptable salt thereof.

11. The Compound of claim 8 where R⁶ is alkyl substituted with one R⁹ independently selected from alkylsulfonyl, hydroxy, —C(O)NR¹⁰R^{10a}, and —C(O)OR¹⁰ or R⁶ is alkyl substituted with two R⁹ where both R⁹ are hydroxy; optionally as a single stereoisomer or a mixture of isomers thereof and all additionally optionally as a pharmaceutically acceptable salt thereof.

12. The Compound of claim 8 where R⁶ is —OR¹³; optionally as a single stereoisomer or a mixture of isomers thereof and all additionally optionally as a pharmaceutically acceptable salt thereof.

13. The Compound of claim 8 where R⁶ is —OR¹³ and R¹³ is heterocycloalkyl optionally substituted with one group independently selected from alkyl, carboxy, carboxyalkyl, and hydroxyalkyl; or R¹³ is alkyl substituted with 1, 2, or 3 groups independently selected from hydroxy, —C(O)R^{10b}, —NR¹¹R^{11a}, —P(O)(OR¹⁶)₂, —OP(O)(OR¹⁶)₂, and —OS(O)₂OH and the R¹³ alkyl is additionally optionally substituted with 1, 2, or 3 halo; optionally as a single stereoisomer or a mixture of isomers thereof and all additionally optionally as a pharmaceutically acceptable salt thereof.

14. The Compound of claim 8 where R⁶ is —OR¹³ and R¹³ is alkyl substituted with 1, 2, or 3 groups independently selected from hydroxy, —C(O)R^{10b}, —NR11_{R}^{11a}, —P(O)(OH)₂, —OP(O)(OH)₂, and —OS(O)₂OH and the R¹³ alkyl is additionally optionally substituted with 1, 2, or 3 halo; optionally as a single stereoisomer or a mixture of isomers thereof and all additionally optionally as a pharmaceutically acceptable salt thereof.

15. The Compound of claim 8 where R⁶ is —NR¹²S(O)₂R^{12a}; optionally as a single stereoisomer or a mixture of isomers thereof and all additionally optionally as a pharmaceutically acceptable salt thereof.

16. The Compound of claim 8 where R⁶ is —NR¹¹R¹¹; optionally as a single stereoisomer or a mixture of isomers thereof and all additionally optionally as a pharmaceutically acceptable salt thereof.

17. The Compound of claim 8 where R⁶ is cycloalkyl optionally substituted with 1 or 2 groups independently selected from hydroxyalkyl, carboxy, and —C(O)NR¹⁰R^{10a}; optionally as a single stereoisomer or a mixture of isomers thereof and all additionally optionally as a pharmaceutically acceptable salt thereof.

18. The Compound according to claim 1 selected from Table 1 as numbered:
```
  1
  2
  3
  4
  5
  6
  7
  8
  9
  10
  11
  12
  13
  14
  15
  16
  17
  18
  19
  20
  21
  22
  23
  24
  25
  26
  28
  29
  30
  31
  32
  33
  34
  35
  36
  37
  38
  39
  41
  42
  43
  44
  45
  46
  47
  48
  49
  50
  51
  52
  53
  54
  56
  57
  58
  59
  60
  61
  62
  63
  64
  65
  66
  67
  68
  69
  70
  71
  72
  73
  75
  76
  77
  78
  79
  80
  81
  82
  83
  84
  85
  86
  87
  88
  89
  90
  91
  92
  93
  94
  95
  96
  97
  98
  99
100
101
102
103
104
105
106
107
109
110
111
112
113
115
116
117
118
119
120
121
122
123
124
126
127
128
129
130
131
132
134
135
136
138
139
140
141
142
143
144
145
146
147
148
149
150
152
153
154
155
156
157
159
160
161
162
163
164
165
166
167
168
170
171
172
173
174
175
176
177
178
179
180
181
182
183
184
185
186
187
188
189
190
191
192
193
194
195
196
197
198
199
200
201
202
203
204
205
206
207
208
209
211
212
213
214
215
216
217
218
219
220
221
222
223
224
225
226
227
228
229
230
232
233
234
235
238
239
240
241
242
243
244
245
246
247
248
249
250
251
252
253
255
256
257
258
259
260
261
262
263
264
265
266
267
268
271
272
273
274
275
276
277
278
279
280
281
282
283
284
285
286
287
288
289
291
292
293
294
295
296
297
298
299
300
301
302
303
304
305
306
307
308
309
310
311
312
313
314
315
316
317
318
319
320
321
322
323
324
325
326
327
328
329
330
331
332
333
334
335
336
337
338
339
420
and
421;
```
optionally as a single stereoisomer or a mixture of isomers thereof and all additionally optionally as a pharmaceutically acceptable salt thereof.

19. The Compound of claim 1 selected from Table 2 as numbered:
343
344
345
346
347
348
349
350
351
352
353
354
355
356
357
358
359
360
361
362
363
364
365
366
367
368
369
370
371
372
373
374
375
376
377
378
379
380
381
382
383
384
385
386
387
388
389
390
391
392
393
394
395
396
397
398
399
400
401
402
403
404
405
406
407
408
409
410
411
412
413
414
415
416
417
418
and
419.

20. A pharmaceutical composition which comprises 1) a compound of claim 1 or 18, optionally as a single stereoisomer or a mixture of isomers thereof and all additionally optionally as a pharmaceutically acceptable salt thereof, and 2) a pharmaceutically acceptable carrier, excipient, or diluent.

21. A method for treating a disease, disorder, or syndrome which method comprises administering to a patient a therapeutically effective amount of a compound of claim 1 or 18, optionally as a single stereoisomer or a mixture of isomers thereof, and all additionally optionally as a pharmaceutically acceptable salt thereof and all optionally with a pharmaceutically acceptable carrier, excipient, or diluent.

22. The method of of claim 21 where the disease is psoriasis.

23. The method of claim 21 where the disease is an autoimmune disease.

24. The method of claim 23 where the autoimmune disease is multiple sclerosis.

25. The method of claim 23 where the autoimmune disease is graft-versus-host disease.

26. The method of claim 23 where the autoimmune disease is autoimmune-induced inflammation.

27. The method of claim 23 where the autoimmune disease is Crohn's disease.

28. A method of making a Compound of claim 1, comprising
(a) reacting an compound of formula (g): 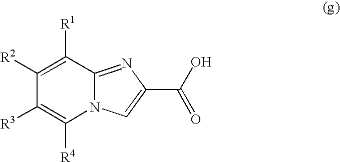
where R¹, R², R³, and R⁴ are as defined in claim 1, with a reagent R⁵C(═NOH)NH₂ (j) where R⁵ is as defined in claim 1; to yield a Compound of Formula I(a): 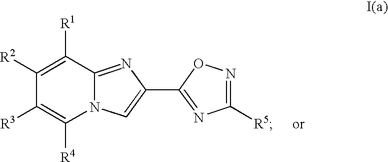
(b) reacting an compound of formula (k): 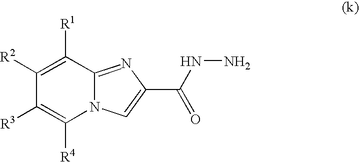
where R¹, R², R³, and R⁴ are as defined in claim 1, with a reagent R⁵C(O)OH (m) where R⁵ is as defined in claim 1 and followed by treatment with EtSH to yield a Compound of Formula I (j): 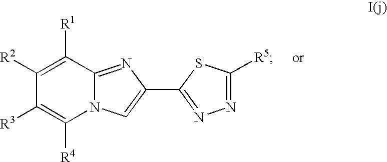
(c) reacting an compound of formula (g) as described above with a reagent of formula R⁵C(O)NHNH₂ (p) where R⁵ is as defined in claim 1 for a Compound of Formula I, to yield a Compound of Formula I (e): 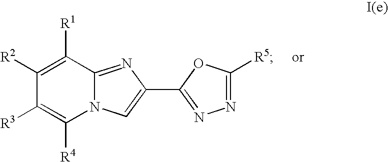
(d) reacting an compound of formula (p): 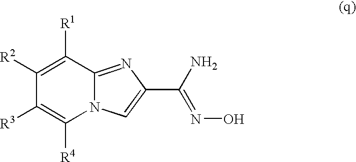
where R¹, R², R³, and R⁴ are as defined in claim 1, with a regent R⁵C(O)OH (r) where R⁵ is as defined in claim 1, to yield a Compound of Formula I (c): 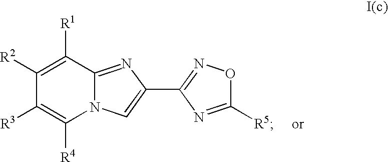
(e) reacting an compound of formula I (n): 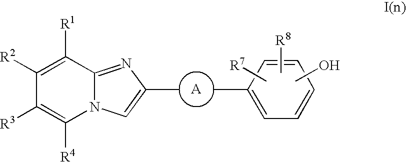
where R¹, R², R³, R⁴, R⁷, and R⁸ are as defined in claim 1, with a reagent of formula R¹³X where X is halo and R¹³ is as defined in claim 1, to yield a Compound of Formula I (p): 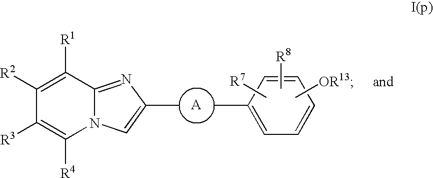
(f) optionally modifying any of R¹, R², R³, R⁴, and R⁵ and the substituents contained therein; and
(g) optionally further resolving individual isomers.
